(19) United States
(12) Patent Application Publication
(10) Pub. No.: US 2026/0152543 A1
(43) Pub. Date: Jun. 4, 2026

(54) COMPLEMENT PATHWAY INHIBITOR FUSIONS AND METHODS OF USING THE SAME

(71) Applicant: Kriya Therapeutics, Inc., Morrisville, NC (US)

(72) Inventors: Qi YANG, Morrisville, NC (US); Spencer KNIGHT, Morrisville, NC (US); Andrew MURPHY, Cary, NC (US); Michele STONE, Apex, NC (US); Nachiketa GUPTA, San Francisco, CA (US); Rachel ECLOV, Morrisville, NC (US)

(21) Appl. No.: 19/418,581

(22) Filed: Dec. 12, 2025

Related U.S. Application Data
(63) Continuation of application No. PCT/US2024/037936, filed on Jul. 12, 2024.
(60) Provisional application No. 63/513,425, filed on Jul. 13, 2023.
(60) Provisional application No. 63/617,671, filed on Jan. 4, 2024.
(60) Provisional application No. 63/641,276, filed on May 1, 2024.

(51) Int. Cl. C07K 14/705; A61K 38/17; A61K 48/00; C07K 14/71; C12N 15/86

(52) U.S. Cl. CPC C07K 14/70596; A61K 38/177; A61K 38/179; A61K 48/005; A61K 48/0075; C07K 14/71; C12N 15/86; C07K 2319/00; C12N 2750/14143

(57) ABSTRACT

According to various aspects of this disclosure, the present disclosure relates to a fusion protein comprising a truncated complement receptor 1 (CR1) protein or a functional fragment thereof. In some aspects, the fusion protein further comprises a complement receptor 2 (CR2) protein or a functional fragment thereof. Certain aspects of this disclosure relate to an adeno-associated virus (AAV) vector comprising a polynucleotide comprising a promoter operably linked to a nucleic acid encoding a CR2-CRI fusion protein. Other aspects of this disclosure relate to a method of treating a subject suffering from a complement-mediated disorder, the method comprising administering any of the fusion proteins disclosed herein or any of the AAV vectors disclosed herein.

### CROSS REFERENCE TO RELATED APPLICATIONS

The present application is a continuation of International Application No. PCT/US2024/037936, filed Jul. 12, 2024, which claims the priority benefit of U.S. Provisional Application No. 63/513,425, filed Jul. 13, 2023; U.S. Provisional Application No. 63/617,671, filed Jan. 4, 2024; and U.S. Provisional Application No. 63/641,276, filed May 1, 2024; each of which is hereby incorporated by reference in its entirety.

### REFERENCE TO SEQUENCE LISTING SUBMITTED ELECTRONICALLY

The content of the electronically submitted sequence listing (Name: 4525_1000004_SequenceListing_ST26.xml; Size: 555,059 bytes; and Date of Creation: Dec. 11, 2025), file with the application, is incorporated herein by reference in its entirety.

### FIELD OF THE DISCLOSURE

The present disclosure relates to the medical field, including protein and nucleic acid therapies for inhibiting the complement pathway.

### BACKGROUND OF THE DISCLOSURE

The complement system is a complex innate immune surveillance system, playing a key role in defense against pathogens and in host homeostasis. The complement system is initiated by conformational changes in recognition molecular complexes upon sensing danger signals. The subsequent cascade of enzymatic reactions is tightly regulated to assure that complement is activated only at specific locations requiring defense against pathogens, thus avoiding host tissue damage.

The complement system is a central part of the innate immunity that serves as a first line of defense against foreign and altered host cells (Ricklin et al., Nat. Immunol. 11:785-797 (2010)). The complement system is composed of plasma proteins produced mainly by the liver or membrane proteins expressed on cell surface. Complement operates in plasma, in tissues, or within cells (Kolev et al., Nat. Rev. Immunol. 14:811-820 (2014)). Complement proteins collaborate as a cascade to opsonize pathogens and induce a series of inflammatory responses helping immune cells to fight infection and maintain homeostasis. The complement system can be initiated depending on the context by three distinct pathways—classical (CP), lectin (LP), and alternative (AP), each leading to a common terminal pathway.

Both inefficient and over stimulation of complement can be detrimental for the host and are associated with increased susceptibility to infections or non-infectious diseases, including autoimmunity, chronic inflammation, thrombotic microangiopathy, graft rejection, and cancer.

### BRIEF SUMMARY OF THE INVENTION

Certain aspects of the present disclosure provides a polypeptide comprising a truncated complement receptor 1 (CR1) protein. In some aspects, the truncated CR1 protein has an amino acid sequence with at least 85%, at least 90%, at least 95%, at least 96%, at least 97%, at least 98%, at least 99%, or 100% identity to any one of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In some aspects, the polypeptide is a fusion protein. In some aspects, fusion protein comprises the truncated CR1 protein and a complement receptor 2 (CR2) protein or a functional fragment thereof. In some aspects, the fusion protein comprises the truncated CR1 protein and a VEGF inhibiting domain (VID), and optionally, a half-life prolonging domain.

Certain aspects of the present disclosure provides a fusion protein comprising i) a complement receptor 2 (CR2) protein or a functional fragment thereof, and ii) a complement receptor 1 (CR1) protein or a functional fragment thereof. In some aspects, the complement receptor 2 protein or functional fragment thereof has an amino acid sequence with at least 85%, at least 90%, at least 95%, at least 96%, at least 97%, at least 98%, at least 99%, or 100% identity to SEQ ID NO: 1 or SEQ ID NO: 2. In some aspects, the complement receptor 1 protein or functional fragment thereof has an amino acid sequence with at least 85%, at least 90%, at least 95%, at least 96%, at least 97%, at least 98%, at least 99%, or 100% identity to any of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In some aspects, the C terminus of the CR2 protein or functional fragment thereof is linked (e.g., by a first linker) to the N terminus of the CR1 protein or functional fragment thereof (e.g., a truncated CR1 protein disclosed herein). In some aspects, the C terminus of the CR1 protein or functional fragment thereof (e.g., a truncated CR1 protein disclosed herein) is linked (e.g., by a first linker) to the N terminus of the CR2 protein or functional fragment thereof. In some aspects, the fusion protein comprises, from the N-terminal to C-terminal, a CR2 protein or functional fragment thereof, a linker (e.g., a first linker), and a CR1 protein or functional fragment thereof (e.g., a truncated CR1 protein disclosed herein), wherein the fusion protein inhibits or reduces complement pathway (e.g., classical and alternative pathway) activation.

In some aspects, the complement receptor 1 protein or functional fragment thereof comprises at least two Short Consensus Repeats (SCRs), wherein the SCRs are linked by a second linker (e.g., also sometimes referred to herein as a spacer).

In some aspects, the first and/or second linker is a (GGGGS)n linker, wherein n is between 2 and 12 (e.g., SEQ ID NOs: 156-160).

In some aspects, the complement receptor 2 protein or functional fragment thereof has an amino acid sequence with at least 85%, at least 90%, at least 95%, at least 96%, at least 97%, at least 98%, at least 99%, or 100% identity to SEQ ID NO: 1 or SEQ ID NO: 2.

In some aspects, the polypeptide comprises a truncated CR1 having an amino acid sequence with at least 85%, at least 90%, at least 95%, at least 96%, at least 97%, at least 98%, at least 99%, or 100% identity to any one of SEQ ID NOs: 3-11 or 148-155.

In some aspects, the complement receptor 1 protein or functional fragment thereof has an amino acid sequence with at least 85%, at least 90%, at least 95%, at least 96%, at least 97%, at least 98%, at least 99%, or 100% identity to any one of SEQ ID NOs: 3-11 or 148-155.

In some aspects, the fusion protein has an amino acid sequence with at least 85%, at least 90%, at least 95%, at least 96%, at least 97%, at least 98%, at least 99%, or 100% identity to any one of SEQ ID NOs: 12-28.

In some aspects, the fusion protein has an amino acid sequence with at least 85%, at least 90%, at least 95%, at least 96%, at least 97%, at least 98%, at least 99%, or 100% identity to SEQ ID NO: 12.

In some aspects, the fusion protein has an amino acid sequence with at least 85%, at least 90%, at least 95%, at least 96%, at least 97%, at least 98%, at least 99%, or 100% identity to SEQ ID NO: 13.

In some aspects, the fusion protein has an amino acid sequence with at least 85%, at least 90%, at least 95%, at least 96%, at least 97%, at least 98%, at least 99%, or 100% identity to SEQ ID NO: 14.

In some aspects, the fusion protein has an amino acid sequence with at least 85%, at least 90%, at least 95%, at least 96%, at least 97%, at least 98%, at least 99%, or 100% identity to SEQ ID NO: 15.

In some aspects, the fusion protein has an amino acid sequence with at least 85%, at least 90%, at least 95%, at least 96%, at least 97%, at least 98%, at least 99%, or 100% identity to SEQ ID NO: 16.

In some aspects, the fusion protein has an amino acid sequence with at least 85%, at least 90%, at least 95%, at least 96%, at least 97%, at least 98%, at least 99%, or 100% identity to SEQ ID NO: 17.

In some aspects, the fusion protein has an amino acid sequence with at least 85%, at least 90%, at least 95%, at least 96%, at least 97%, at least 98%, at least 99%, or 100% identity to SEQ ID NO: 18.

In some aspects, the fusion protein has an amino acid sequence with at least 85%, at least 90%, at least 95%, at least 96%, at least 97%, at least 98%, at least 99%, or 100% identity to SEQ ID NO: 19.

In some aspects, the fusion protein has an amino acid sequence with at least 85%, at least 90%, at least 95%, at least 96%, at least 97%, at least 98%, at least 99%, or 100% identity to SEQ ID NO: 20.

In some aspects, the fusion protein has an amino acid sequence with at least 85%, at least 90%, at least 95%, at least 96%, at least 97%, at least 98%, at least 99%, or 100% identity to SEQ ID NO: 21.

In some aspects, the fusion protein has an amino acid sequence with at least 85%, at least 90%, at least 95%, at least 96%, at least 97%, at least 98%, at least 99%, or 100% identity to SEQ ID NO: 22.

In some aspects, the fusion protein has an amino acid sequence with at least 85%, at least 90%, at least 95%, at least 96%, at least 97%, at least 98%, at least 99%, or 100% identity to SEQ ID NO: 23.

In some aspects, the fusion protein has an amino acid sequence with at least 85%, at least 90%, at least 95%, at least 96%, at least 97%, at least 98%, at least 99%, or 100% identity to SEQ ID NO: 24.

In some aspects, the fusion protein has an amino acid sequence with at least 85%, at least 90%, at least 95%, at least 96%, at least 97%, at least 98%, at least 99%, or 100% identity to SEQ ID NO: 25.

In some aspects, the fusion protein has an amino acid sequence with at least 85%, at least 90%, at least 95%, at least 96%, at least 97%, at least 98%, at least 99%, or 100% identity to SEQ ID NO: 26.

In some aspects, the fusion protein has an amino acid sequence with at least 85%, at least 90%, at least 95%, at least 96%, at least 97%, at least 98%, at least 99%, or 100% identity to SEQ ID NO: 27.

In some aspects, the fusion protein has an amino acid sequence with at least 85%, at least 90%, at least 95%, at least 96%, at least 97%, at least 98%, at least 99%, or 100% identity to SEQ ID NO: 28.

Certain aspects of the disclosure are directed to a nucleic acid sequence encoding a fusion protein disclosed here (e.g., a fusion protein having an amino acid sequence selected from any one of SEQ ID Nos: 12-28).

In some aspects, provided herein is a nucleic acid having at least 85%, at least 90%, at least 95%, at least 96%, at least 97%, at least 98%, at least 99%, or 100% identity to any of SEQ ID NOs: 29 to 61.

In some aspects, provided herein is a nucleic acid encoding a CR2-CR1 fusion protein, wherein the nucleic acid has at least 85%, at least 90%, at least 95%, at least 96%, at least 97%, at least 98%, at least 99%, or 100% identity to any of SEQ ID NOs: 77 to 109.

In some aspects, provided herein is a nucleic acid encoding a CR2-CR1 fusion protein, wherein the nucleic acid has at least 85%, at least 90%, at least 95%, at least 96%, at least 97%, at least 98%, at least 99%, or 100% identity to any of SEQ ID NOs: 110 to 113.

In some aspects, provided herein is a nucleic acid encoding a CR2-CR1 fusion protein, wherein the nucleic acid encoding the CR1 polypeptide portion has at least 85%, at least 90%, at least 95%, at least 96%, at least 97%, at least 98%, at least 99%, or 100% identity to any of SEQ ID NOs: 114 to 135.

In some aspects, provided herein is a nucleic acid having at least 85%, at least 90%, at least 95%, at least 96%, at least 97%, at least 98%, at least 99%, or 100% identity to any of SEQ ID NOs: 141 or 142.

In some aspects, provided herein is a vector for delivering any of the nucleic acids disclosed herein to a subject, wherein the vector is a viral vector or a non-viral vector.

In some aspects, the viral vector is selected from the group consisting of an adenoviral vector, an adeno-associated virus (AAV) vector, or a lentiviral vector.

In some aspects, provided herein is an adeno-associated virus (AAV) vector comprising a polynucleotide comprising a promoter operably linked to a nucleic acid encoding a CR2-CR1 fusion protein disclosed herein. In some aspects, the nucleic acid encoding the CR2-CR1 fusion comprises any of the nucleic acids disclosed herein.

In some aspects, the polynucleotide comprises a nucleic acid having at least 85%, at least 90%, at least 95%, at least 96%, at least 97%, at least 98%, at least 99%, or 100% identity to SEQ ID NOs: 29 to 61.

In some aspects, the nucleic acid encoding the CR2-CR1 fusion protein comprises a nucleic acid having at least 85%, at least 90%, at least 95%, at least 96%, at least 97%, at least 98%, at least 99%, or 100% identity to any of SEQ ID NOs: 77-109.

In some aspects, the nucleic acid encoding the CR2-CR1 fusion protein comprises a nucleic acid having at least 85%, at least 90%, at least 95%, at least 96%, at least 97%, at least 98%, at least 99%, or 100% identity to any of SEQ ID NO: 110-113.

In some aspects, the nucleic acid encoding the CR1 portion of the CR2-CR1 fusion protein comprises a nucleic acid having at least 85%, at least 90%, at least 95%, at least 96%, at least 97%, at least 98%, at least 99%, or 100% identity to any of SEQ ID NO: 114 to 135.

Certain aspects of the present disclosure provides a fusion protein comprising i) a VEGF inhibiting domain (VID) and ii) a complement receptor 1 (CR1) protein or functional fragment thereof. In some aspects, the CR1 protein or functional fragment thereof has an amino acid sequence with at least 85%, at least 90%, at least 95%, at least 96%, at least 97%, at least 98%, at least 99%, or 100% identity to any of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In some aspects, the VID has an amino acid sequence with at least 85%, at least 90%, at least 95%, at least 96%, at least 97%, at least 98%, at least 99%, or 100% identity to any of SEQ ID NO: 136, 137, 146, or 147.

In some aspects, the fusion protein further comprises a half-life prolonging domain (e.g., an immunoglobulin Fc region). In some aspects, the half-life prolonging domain is an IgG1 Fc domain or fragment thereof comprising an amino acid sequence having at least 85%, at least 90%, at least 95%, at least 96%, at least 97%, at least 98%, at least 99%, or 100% identity to any of SEQ ID NO: 138 or 143-145.

In some aspects, the C terminus of the VID is linked to the N terminus of the CR1 protein or functional fragment thereof by a linker. In some aspects, the C terminus of the CR1 protein or functional fragment thereof is linked to the N terminus of the VID by a linker. In some aspects, the fusion protein comprises, from the N-terminal to C-terminal, a VEGF inhibiting domain (VID), optionally an immunoglobulin Fc region, optionally a linker, and a truncated CR1 protein, wherein the fusion protein inhibits or reduces complement pathway activation and VEGF activity.

In some aspects, provided herein is a nucleic acid encoding a VID-CR1 fusion protein, wherein the CR1 portion of the VID-CR1 fusion protein has an amino acid sequence with at least 85%, at least 90%, at least 95%, at least 96%, at least 97%, at least 98%, at least 99%, or 100% identity to any of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In some aspects, the VID of the VID-CR1 fusion protein has an amino acid sequence with at least 85%, at least 90%, at least 95%, at least 96%, at least 97%, at least 98%, at least 99%, or 100% identity to any of SEQ ID NO: 136, 137, 146, or 147.

In some aspects, the VID-CR1 fusion protein further comprises a linker having an amino acid sequence with at least 95%, at least 96%, at least 97%, at least 98%, at least 99%, or 100% identity to SEQ ID NO: 139.

In some aspects, the fusion protein comprises an amino acid sequence having at least 85%, at least 90%, at least 95%, at least 96%, at least 97%, at least 98%, at least 99%, or 100% identity to SEQ ID NO: 140.

In some aspects, provided herein is a vector for delivering any of the nucleic acids disclosed herein to a subject, wherein the vector is a viral vector or a non-viral vector.

In some aspects, the viral vector is selected from the group consisting of an adenoviral vector, an adeno-associated virus (AAV) vector, or a lentiviral vector.

In some aspects, provided herein is an adeno-associated virus (AAV) vector comprising a polynucleotide comprising a promoter operably linked to a nucleic acid encoding a VID-CR1 fusion protein disclosed herein.

In some aspects, the promoter (e.g., operably liked to a nucleic acid encoding a CR2-CR1 or VID-CR1 fusion protein disclosed herein) comprises a CBA promoter, a CMV promoter, an EF-1a (Elongation Factor 1a) promoter, a RSV (Rous Sarcoma Virus) promoter, an Ubiquitin (UbC) promoter, a CAG promoter, a smCBA promoter or any combination thereof.

In some aspects, the promoter is a smCBA promoter.

In some aspects, the polynucleotide further comprises a kozak sequence.

In some aspects, the polynucleotide further comprises a cytomegalovirus (CMV) immediate-early enhancer and/or promoter.

In some aspects, the polynucleotide further comprises a poly(A) sequence.

In some aspects, the poly(A) sequence comprises a human growth hormone poly(A) sequence, a bovine growth hormone poly(A) sequence, or a synthetic poly(A) sequence.

In some aspects, the poly(A) sequence is a synthetic poly(A) sequence.

In some aspects, the synthetic poly(A) sequence comprises a nucleic acid sequence corresponding to SEQ ID NO: 71.

In some aspects, the poly(A) sequence is a bovine growth hormone poly(A) sequence.

In some aspects, the bovine growth hormone poly(A) sequence comprises a nucleic acid sequence corresponding to SEQ ID NO: 72.

In some aspects, the poly(A) sequence is a human growth hormone poly(A) sequence.

In some aspects, the human growth hormone poly(A) sequence comprises a nucleic acid sequence corresponding to SEQ ID NO: 73.

In some aspects, the polynucleotide further comprises two inverted terminal repeat (ITR) sequences.

In some aspects, the polynucleotide further comprises an enhancer sequence.

In some aspects, the enhancer is a CMV enhancer sequence.

In some aspects, polynucleotide further comprises an intron sequence.

In some aspects, the intron sequence is a SV40 intron sequence or a CAG intron sequence.

In some aspects, the AAV comprises an AAV serotype capsid selected from the group consisting of AAV1, AAV2, AAV3, AAV4, AAV5, AAV6, AAV7, AAV8, AAVrh8, AAV9, AAV10, AAVrh10, AAV11, and AAV12.

In some aspects, the AAV vector comprises an AAV2 serotype capsid.

In some aspects, provided herein is a method of treating a subject suffering from a complement-mediated disorder selected from: (i) various disorders characterized by hemolysis or hemolytic anemia such as atypical hemolytic uremic syndrome, cold agglutinin disease, paroxysmal nocturnal hemoglobinuria, transfusion reactions; (ii) transplant rejection (e.g., hyperacute or acute transplant rejection) or transplant dysfunction; (iii) disorders involving ischemia/reperfusion injury such as trauma, surgery (e.g., aneurysm repair), myocardial infarction, ischemic stroke; (iv) disorders of the respiratory system such as asthma and chronic obstructive pulmonary disease (COPD); (v) arthritis, e.g., rheumatoid arthritis; or (vi) ocular disorders such as age-related macular degeneration (AMD), diabetic retinopathy, glaucoma, and uveitis or macular degeneration, the method comprising administering an effective amount of any of the fusion proteins disclosed herein (e.g., a CR2-CR1 fusion protein), any of the nucleic acids disclosed herein, any of the vectors disclosed herein, or any of the AAV vectors disclosed herein.

In some aspects, provided herein is a method of treating a subject suffering from a) a solid tumor associated with angiogenesis or b) an ocular disease or condition associated with abnormal blood vessel in an eye, comprising administering to the subject an effective amount of any of the fusion proteins disclosed herein (e.g., a CR1-VID fusion protein), any of the nucleic acids disclosed herein, any of the vectors disclosed herein, or any of the AAV vectors disclosed herein. In some aspects, the ocular disease or condition associated with abnormal blood vessel in an eye is diabetic retinopathy. In some aspects, the ocular disease or condition associated with abnormal blood vessel in an eye is wet age-related macular degeneration. In some aspects, the subject has breast cancer, colorectal cancer, lung cancer, kidney cancer, gastric cancer, ovarian cancer, or retinoblastoma.

In some aspects, the fusion protein, the polynucleotide, the vector, or the AAV vector is administered to an eye of the subject using intravitreal, subretinal, or suprachoroidal injection. In some aspects, the fusion protein, the polynucleotide, the vector, or the AAV vector is administered to an eye of the subject by suprachoroidal injection.

In some aspects, the fusion protein is administered to eye tissue, wherein the eye tissue comprises retinal pigment epithelium (RPE) or choroid.

In some aspects, the macular degeneration comprises age-related macular degeneration (AIMD).

In some aspects, the ratio of C3b:iC3b is reduced in the subject (e.g., the eye of the subject) after the administration of any of the fusion proteins disclosed herein (e.g., a CR2-CR1 fusion protein), any of the nucleic acids disclosed herein, any of the vectors disclosed herein, any of the AAV vectors disclosed herein, or any of the pharmaceutical compositions disclosed herein.

In some aspects, provided herein is a polynucleotide comprising a nucleic acid sequence encoding any of the fusion protein disclosed herein.

In some aspects, provided herein is a polynucleotide comprising comprises a nucleic acid sequence having at least 85%, at least 90%, at least 95%, at least 96%, at least 97%, at least 98%, at least 99%, or 100% identity to any of SEQ ID NOs: 29-61 or 77-135.

In some aspects, provided herein is an adeno-associated virus (AAV) capsid comprising any of the nucleic acids disclosed herein, or any of the polynucleotides disclosed herein.

In some aspects, provided herein is a recombinant adeno-associated virus (rAAV) vector comprising an AAV capsid encapsidating any of the nucleic acids disclosed herein, or any of the polynucleotides disclosed herein.

In some aspects, the AAV capsid serotype is selected from the group consisting of AAV1, AAV2, AAV3, AAV4, AAV5, AAV6, AAV7, AAV8, AAVrh8, AAV9, AAV10, AAVrh10, AAV11, and AAV12.

In some aspects, the AAV capsid serotype is selected from AAV2, AAV5, or AAV8. In some aspects, the AAV capsid serotype is AAV2.

In some aspects, the AAV capsid is modified relative to the wild-type serotype.

In some aspects, provided herein is a pharmaceutical composition comprising any of the fusion proteins disclosed herein, any of the nucleic acids disclosed herein, any of the vectors disclosed herein, any of the AAV vectors disclosed herein, any of the polynucleotides disclosed herein, any of the AAV capsids, or any rAAV vector disclosed herein.

In some aspects, the pharmaceutical composition further comprises a pharmaceutically acceptable excipient.

In some aspects, provided herein is the use of any of the fusion proteins disclosed herein, any of the nucleic acids disclosed herein, any of the vectors disclosed herein, any of the AAV vectors (e.g., rAAV vectors) disclosed herein, any of the polynucleotides disclosed herein, any of the AAV capsids disclosed herein, or any of the pharmaceutical compositions disclosed herein for the treatment of a subject suffering from a complement-mediated disorder selected from: (i) various disorders characterized by hemolysis or hemolytic anemia such as atypical hemolytic uremic syndrome, cold agglutinin disease, paroxysmal nocturnal hemoglobinuria, transfusion reactions; (ii) transplant rejection (e.g., hyperacute or acute transplant rejection) or transplant dysfunction; (iii) disorders involving ischemia/reperfusion injury such as trauma, surgery (e.g., aneurysm repair), myocardial infarction, ischemic stroke; (iv) disorders of the respiratory system such as asthma and chronic obstructive pulmonary disease (COPD); (v) arthritis, e.g., rheumatoid arthritis; or (vi) ocular disorders such as age-related macular degeneration (AMD), diabetic retinopathy, glaucoma, and uveitis.

### DESCRIPTION OF FIGURES

FIGS. 1A-1B show a schematic of Complement Receptor 2 (“CR2”) (FIG. 1A) and Complement Receptor 1 (“CR1”) (FIG. 1).

FIGS. 2A-2M show exemplary fusion protein constructs of CR2 and CR1.

FIG. 3 shows mRNA expression of CR2-CR1 fusion constructs as measured by RT-qPCR.

FIGS. 4A-4D show protein expression of CR2-CR1 fusion constructs in HEK293 cells as measured by Gyros Immunoassay.

FIG. 5 shows protein expression of CR2-CR1 fusion constructs as grouped by the number of Spacer Short Consensus Repeats (“SCRs”).

FIGS. 6A-6G show inhibition of the classical complement pathway by recombinant human CR1 protein, and CR2-CR1 fusion constructs (CR2CR1 #1, CR2CR1 #2, CR2CR1 #5, CR2CR1 #6, CR2CR1 #7, CR2CR1 #8, CR2CR1 #9, and CR2CR1 #10) as measured by sensitized sheep Red Blood cell lysis with Human Serum.

FIGS. 7A-7C show inhibition of the classical complement pathway by recombinant human CR1 protein, and CR2-CR1 fusion constructs (CR2CR1 #1, CR2CR1 #2, CR2CR1 #7, CR2CR1 #8, CR2CR1 #9, CR2CR1 #10, and CR2CR1 #13) as measured by a Wieslab® complement system classical pathway assay.

FIGS. 8A-8B show inhibition of the alternative complement pathway by recombinant human CR1 protein, and CR2-CR1 fusion constructs (CR2CR1 #1, CR2CR1 #2, CR2CR1 #7, CR2CR1 #8, and CR2CR1 #10) as measured by a Wieslab® complement system alternative pathway assay.

FIGS. 9A-9B show inhibition of the alternative complement pathway by recombinant human CR1 protein, and two CR2-CR1 fusion constructs (CR2CR1 #1 and CR2CR1 #7) as measured by a Wieslab® complement system classical pathway assay.

FIG. 10 shows measurement of CR2-CR1 fusion protein expression in mouse ocular homogenate using a dual CR2-CR1 Immunoassay after administration of rAAV CR2CR1-#7 with 8E8 vg/eye or 1E10 vg/eye.

FIG. 11 shows a bar graph of the lesion area (mm²) as quantified via retinal flatmount for vehicle, CR2-CR1 #7 low dose, and CR2-CR1 #7 high dose.

FIGS. 12A-12B show bar graphs of Outer Nuclear Layer (ONL) thickness and % change from day 25 (CFD25) for both the superior retina (FIG. 12A) and inferior retina (FIG. 12B) per left (OS) and right (OD) eyes across vehicle, CR2-CR1 #7 low dose, and CR2-CR1 #7 high dose groups.

FIGS. 13A-13B show bar graphs of Total Retinal thickness and % change from day 25 (CFD25) for both the superior retina (FIG. 13A) and inferior retina (FIG. 13B) per left (OS) and right (OD) eyes across vehicle, CR2-CR1 #7 low dose, and CR2-CR1 #7 high dose groups.

FIGS. 14A-14D show Full-field Electroretinography (ff-ERG) scotopic (dark adapted) a-wave amplitude (uV) measurements (FIG. 14A), a-wave amplitude changes from baseline (FIG. 14B), b-wave amplitude measurements (FIG. 14C), and b-wave amplitude changes from baseline (FIG. 14D) at day 38.

FIG. 15 shows VG/mRNA levels of CR2-CR1 #7. LLOQ: 3.06 pg/mg (1.53 pg/mL) and CR2-CR1 #1 used as calibrator protein.

FIGS. 16A-16B show C3b and iC3b protein Western blot analyses from naïve, vehicle or AAV2.CR2-CR1 #7 vector treated animals following NaIO₃ treatment (FIG. 16A), and the ratio of calculated protein amounts (FIG. 16B).

FIGS. 17A-17B show optical coherence tomography (OCT) images of the superior B-scan (FIG. 17A) and central B-scan (FIG. 17B) demonstrating fluid in the suprachoroidal space (SCS) immediately after administration of AAV2.CR2-CR1 #7 in non-human primates.

FIG. 18 shows intraocular pressure (IOP) measurements through 85 days post-SCS administration of AAV2.CR2-CR1 #7 in non-human primates.

FIGS. 19A-19B shows vector genome (vg) (FIG. 19A) and transgene mRNA (FIG. 19B) biodistribution of AAV2.CR2-CR1 #7 following bilateral SCS administration to non-human primates.

FIG. 20 shows systemic biodistribution of AAV2.CR2-CR1 #7 following bilateral SCS administration to non-human primates.

FIGS. 21A-21C show surface plasmon resonance (SPR) sensograms generated using the analyte recombinant C3b, with recombinant human CR1 as the ligand (FIG. 21A), recombinant CR2CR1 #1 as the ligand (FIG. 21B), or recombinant CR2CR1 #7 as the ligand (FIG. 21C).

### DETAILED DESCRIPTION

In some aspects, provided herein is a polypeptide comprising a truncated complement receptor 1 (CR1) protein. In some aspects, the truncated CR1 protein has an amino acid sequence with at least 85%, at least 90%, at least 95%, at least 96%, at least 97%, at least 98%, at least 99%, or 100% identity to any one of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In some aspects, the polypeptide is a fusion protein. In some aspects, fusion protein comprises the truncated CR1 protein and a complement receptor 2 (CR2) protein or a functional fragment thereof. In some aspects, the fusion protein comprises the truncated CR1 protein and a VEGF inhibiting domain (VID), and optionally, a half-life prolonging domain.

In some aspects, provided herein is a fusion protein comprising (i) a complement receptor 2 (CR2) protein or a functional fragment thereof and (ii) a complement receptor 1 (CR1) protein or a functional fragment thereof (CR2-CR1 fusion protein). In some aspects, the CR2 protein or functional fragment thereof comprises an amino acid sequence having at least 85%, at least 90%, at least 95%, at least 96%, at least 97%, at least 98%, at least 99%, or 100% identity to SEQ ID NO: 1 or SEQ ID NO: 2. In some aspects, the CR1 protein or functional fragment thereof comprises an amino acid sequence having at least 85%, at least 90%, at least 95%, at least 96%, at least 97%, at least 98%, at least 99%, or 100% identity to any of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In some aspects, provided herein is a polynucleotide comprising a promoter operably linked to a nucleic acid encoding a CR2-CR1 fusion protein. In some aspects, the polynucleotide comprises a nucleic acid sequence having at least 85%, at least 90%, at least 95%, at least 96%, at least 97%, at least 98%, at least 99%, or 100% identity to any of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, or SEQ ID NO: 61.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to any of SEQ ID NOs: 77-109.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to any of SEQ ID NOs: 110-113.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to any of SEQ ID NOs: 114-135.

In some aspects, provided herein is an adeno-associated virus (AAV) vector comprising a pair of AAV inverted terminal repeats (ITRs) (e.g., 3′ and 5′ ITRs) and a polynucleotide (e.g., an expression cassette) comprising a promoter operably linked to a nucleic acid encoding a CR2-CR1 fusion protein. In some aspects, the polynucleotide comprises a nucleic acid sequence having at least 85%, at least 90%, at least 95%, at least 96%, at least 97%, at least 98%, at least 99%, or 100% identity to any of SEQ ID NOs: 29-61.

In some aspects, the nucleic acid encoding the CR2-CR1 fusion comprises a nucleic acid sequence having at least 85%, at least 90%, at least 95%, at least 96%, at least 97%, at least 98%, at least 99%, or 100% identity to any of SEQ ID NOs: 77-109. In some aspects, the pair of ITRs is or is derived from an AAV serotype selected from AAV1, AAV2, AAV3a, AAV3b, AAV4, AAV5, AAV6, AAV7, AAV8, AAV9, AAV10, AAV11, AAV12, or variants thereof. In some aspects, the pair of ITRs is or is derived from an AAV2 serotype.

In some aspects, provided herein is a method of treating a subject suffering from a complement-mediated disorder selected from: (i) various disorders characterized by hemolysis or hemolytic anemia such as atypical hemolytic uremic syndrome, cold agglutinin disease, paroxysmal nocturnal hemoglobinuria, transfusion reactions; (ii) transplant rejection (e.g., hyperacute or acute transplant rejection) or transplant dysfunction; (iii) disorders involving ischemia/reperfusion injury such as trauma, surgery (e.g., aneurysm repair), myocardial infarction, ischemic stroke; (iv) disorders of the respiratory system such as asthma and chronic obstructive pulmonary disease (COPD); (v) arthritis, e.g., rheumatoid arthritis; or (vi) ocular disorders such as age-related macular degeneration (AMD), diabetic retinopathy, glaucoma, and uveitis, the method comprising administering any of the fusion proteins disclosed herein or any of the AAV vectors disclosed herein.

In some aspects, the method comprises administering a delivery vector comprising a nucleic acid sequence encoding the fusion protein. In some aspects, the delivery vector is selected from the group consisting of a viral vector (e.g., an AAV vector), a plasmid, a lipid, a protein particle, a bacterial vector, a lysosome, a virus-like particle, a polymeric particle, an exosome, or a vault particle. In some aspects, the delivery vector is a viral vector. In some aspects, the viral vector is an Adeno-associated virus (AAV) vector. In some aspects, the viral vector includes a retroviral vector. In some aspects, the retroviral vector is a lentiviral vector. In some aspects, the viral vector is a herpes virus vector, a polyoma virus vector or a vaccinia virus vector.

In some aspects, provided herein is a polynucleotide comprising a nucleic acid sequence encoding any of the fusion proteins disclosed herein.

In some aspects, provided herein is an adeno-associated (AAV) capsid comprising any of the polynucleotides disclosed herein.

In some aspects, provided herein is a recombinant adeno-associated virus (rAAV) vector comprising an AAV capsid encapsidating any of the nucleic acids disclosed herein, or any of the polynucleotides disclosed herein.

In some aspects, provided herein is a pharmaceutical composition comprising any of the fusion proteins disclosed herein, any of the AAV vectors disclosed herein, any of the polynucleotides disclosed herein, or any of the AAV capsids disclosed herein.

In some aspects, the ratio of C3b:iC3b is reduced in the subject (e.g., the eye of the subject) after the administration of any of the fusion proteins disclosed herein (e.g., a CR2-CR1 fusion protein), any of the nucleic acids disclosed herein, any of the vectors disclosed herein, any of the AAV vectors disclosed herein, or any of the pharmaceutical compositions disclosed herein. In some aspects, C3b is reduced in the subject (e.g., the eye of the subject) after the administration any of the fusion proteins disclosed herein (e.g., a CR2-CR1 fusion protein), any of the nucleic acids disclosed herein, any of the vectors disclosed herein, any of the AAV capsids disclosed herein, any of the AAV vectors (e.g., rAAV vectors) disclosed herein, or any of the pharmaceutical compositions disclosed herein. In some aspects, inactive C3b (iC3b) is increased in the subject (e.g., the eye of the subject) after the administration any of the fusion proteins disclosed herein (e.g., a CR2-CR1 fusion protein), any of the nucleic acids disclosed herein, any of the vectors disclosed herein, any of the AAV capsids disclosed herein, any of the AAV vectors (e.g., rAAV vectors) disclosed herein, or any of the pharmaceutical compositions disclosed herein. In some aspects, C3b is reduced and iC3b is increased in the subject (e.g., the eye of the subject) after the administration of any of the fusion proteins disclosed herein (e.g., a CR2-CR1 fusion protein), any of the nucleic acids disclosed herein, any of the vectors disclosed herein, any of the AAV vectors disclosed herein, or any of the pharmaceutical compositions disclosed herein.

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition. Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21st edition, Gennaro, Ed., Lippincott Williams & Wilkins (2005).

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The term “promoter” as used herein refers to a DNA sequence recognized by the machinery of the cell, or introduced synthetic machinery, required to initiate the transcription of a gene or coding sequence. The term “promoter” is also meant to encompass those nucleic acid elements sufficient for promoter-dependent gene or coding sequence expression controllable for cell-type specific, tissue-specific or inducible by external signals or agents; such elements can be located in the 5′ or 3′ regions of the gene. In some aspects, the promoter is a constitutively active promoter, a cell-type specific promoter, or an inducible promoter.

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene or coding sequence. In some aspects, the term “operably linked” means that a DNA sequence and a regulatory sequence(s) are connected in such a way as to permit gene or coding sequence expression when the appropriate molecules (e.g., transcriptional activator proteins) are bound to the regulatory sequence(s). In some aspects, the term “operably inserted” means that the DNA of interest introduced into the cell is positioned adjacent a DNA sequence which directs transcription and translation of the introduced DNA (i.e., facilitates the production of, e.g., a fusion protein encoded by a DNA of interest).

The term “expression vector”, “expression construct” or “expression cassette” means any type of genetic construct containing a nucleic acid in which part or all of the nucleic acid encoding sequence is capable of being transcribed. Typically, an expression cassette comprises a promoter operably linked to a nucleic acid (e.g., a gene of interest).

The term “adeno-associated virus vector” or “AAV vector” as used herein refers to any vector that comprises or derives from components of an adeno-associated virus. The term AAV vector can be used to designate a vector comprising an AAV genome sequence (e.g., an AAV ITR) and a payload. In some aspects, the AAV vector is suitable to infect mammalian cells, preferably human cells. The term AAV vector can be used to designate an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV capsid” and “AAV particle” can be used interchangeably. In some aspects, the AAV vector is modified or mutated relative to the wild-type AAV serotype sequence. In some aspects, the AAV particle that can comprise an AAV vector comprising at least one payload region (e.g., a polynucleotide encoding a gene of interest) and at least one AAV inverted terminal repeat (ITR) region, which is suitable to infect mammalian cells, preferably human cells.

As used herein, recombinant AAV vectors or rAAV vectors refer to AAV particles lacking viral genes and packaged with payload DNA encoding heterologous genes of interest. In some aspects, the rAAV vectors of the disclosure comprise a polynucleotide encoding a gene of interest flanked by ITRs, which is encapsulated in an AAV particle.

“scAAV” or “self-complementary adeno-associated virus” refers to an adeno-associated viral vector that comprises duplex nucleic acid sequence that are complementary to one another. The two complementary sequences will associate to form one double stranded DNA unit for replication and transcription.

As used herein, and unless otherwise indicated, the term “complementary,” when used to describe a first nucleotide or nucleoside sequence in relation to a second nucleotide or nucleoside sequence, refers to the ability of an oligonucleotide or polynucleotide comprising the first nucleotide or nucleoside sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can be used. The skilled person will be able to determine the set of conditions most appropriate for a test of complementarity of two sequences in accordance with the ultimate application of the hybridized nucleotides or nucleosides.

Complementary sequences can include, or be formed entirely from, non-Watson-Crick base pairs and/or base pairs formed from non-natural and alternative nucleotides or nucleosides, as far as the above requirements with respect to their ability to hybridize are fulfilled. Such non-Watson-Crick base pairs include, but are not limited to, G:U Wobble or Hoogstein base pairing. Complementary sequences within a dsRNA, or between an oligonucleotide and a target sequence as described herein, include base-pairing of the oligonucleotide or polynucleotide comprising a first nucleotide or nucleoside sequence to an oligonucleotide or polynucleotide comprising a second nucleotide or nucleoside sequence over the entire length of one or both nucleotide or nucleoside sequences. Such sequences can be referred to as “fully complementary” or “100% complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., reduction of expression via a RISC pathway. “Substantially complementary” can refer to a polynucleotide that is substantially complementary to a contiguous portion of the nucleic acid of interest (e.g., a DNA or an mRNA encoding a fusion protein). For example, a polynucleotide is complementary to at least a part of a nucleic acid molecule if the sequence is substantially complementary to a non-interrupted portion of a nucleic acid molecule encoding a therapeutic molecule. However, where two oligonucleotides are designed to form, upon hybridization, one or more single stranded overhangs, such overhangs shall not be regarded as mismatches with regard to the determination of complementarity. For example, a shRNA comprising one oligonucleotide of 21 linked nucleosides in length and another oligonucleotide of 23 nucleosides in length, wherein the longer oligonucleotide comprises a sequence of 21 linked nucleosides that is fully complementary to the shorter oligonucleotide, can be referred to as “fully complementary” for the purposes described herein.

“Inverted terminal repeat” (abbreviated: ITR) refers to a single stranded sequence of nucleotides, typically, followed downstream by its reverse complement sequence or a sequence similar to the reverse complement. Naturally occurring ITR sequences are approximately 145 bases each (AAV-1 has ITRs of 143 nucleotides), and AAV plasmids typically include two ITR sequences. The DNA sequence between the ITRs is packaged into the AAV molecule. The intervening sequence of nucleotides between the initial sequence and the reverse complement (or a sequence similar to the reverse complement) can be any length. ITRs can adopt orientations at the ends of the AAV genome that are described, e.g., as FLIP/FLOP, FLOP/FLIP, FLIP/FLIP, or FLOP/FLOP.

“Flanking” or “flanked by” as used herein in the context of the relationship or location of sequences relative to one another, refers to one or more sequences (e.g., ITRs) being on each or on one side of another sequence (e.g., an expression cassette). In some aspects, a sequence can flank another sequence that is immediately beside the sequence. In some aspects, a sequence can flank another sequence where there is an intervening sequence between the sequences.

A “coding sequence” or a sequence “encoding” a particular molecule (e.g., a therapeutic molecule) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

As used herein, the term “synthetic” means designed, produced, prepared, and/or manufactured by the hand of man. Synthesis of polynucleotides or polypeptides or other molecules of the present disclosure can be chemical or enzymatic.

“Nucleic acid,” “polynucleotide,” and “oligonucleotide,” are used interchangeably in the present application. These terms refer to the primary structure of the molecule. Thus, these terms include double- and single-stranded DNA, as well as double- and single-stranded RNA. The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made, e.g., recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides. The disclosure of a polynucleotide or nucleic acid sequence can be understood to disclose the complementary sequences as well.

A kilobase (abbreviated: Kb) is a unit of length of DNA that is equal to 1,000 nucleotide bases (also referred to herein as nucleotides or bases).

The term “mRNA,” as used herein, refers to a single stranded messenger RNA that encodes the amino acid sequence of one or more polypeptide chains.

“Introns” are noncoding sections of an RNA transcript, or the DNA encoding it, that are spliced out before the RNA molecule is translated into a protein.

The phrase “contacting a cell” (e.g., contacting a cell with the fusion proteins, the AAV vectors, the AAV capsids, or the pharmaceutical compositions of the disclosure) as used herein, includes contacting a cell directly or indirectly. In some aspects, contacting a cell with the fusion protein, the AAV vector, the AAV capsid, or the pharmaceutical composition includes contacting a cell in vitro with the fusion protein, the AAV vector, the AAV capsid, or the pharmaceutical composition, or contacting a cell in vivo with the fusion protein, the AAV vector, the AAV capsid, or the pharmaceutical composition. Thus, for example, the fusion protein, the AAV vector, the AAV capsid, or the pharmaceutical composition can be put into physical contact with the cell by the individual performing the method, or alternatively, the fusion protein, the AAV vector, the AAV capsid, or the pharmaceutical composition can be put into a situation that will permit or cause it to subsequently come into contact with the cell.

In some aspects, contacting a cell in vitro can be done, for example, by incubating the cell the fusion protein, the AAV vector, the AAV capsid, or the pharmaceutical composition. In some aspects, contacting a cell in vivo can be done, for example, by injecting the fusion protein, the AAV vector, the AAV capsid, or the pharmaceutical composition of the disclosure into or near the tissue where the cell is located (e.g., the eye), or by injecting the fusion protein, the AAV vector, the AAV capsid, or the pharmaceutical composition into another area, such that the agent will subsequently reach the tissue where the cell to be contacted is located. For example, the AAV vector can be encapsulated and/or coupled to a ligand that directs the AAV vector to a site of interest. Combinations of in vitro and in vivo methods of contacting are also possible. For example, a cell can be contacted in vitro with the fusion protein, the AAV vector, the AAV capsid, or the pharmaceutical composition and subsequently transplanted into a subject.

In some aspects, contacting a cell with the fusion protein, the AAV vector, the AAV capsid, or the pharmaceutical composition of the present disclosure includes “introducing” or “delivering” (directly or indirectly) the fusion protein, the AAV vector, the AAV capsid, or the pharmaceutical composition into the cell by facilitating or effecting uptake or absorption into the cell. Introducing the fusion protein, the AAV vector, the AAV capsid, or the pharmaceutical composition into a cell can be in vitro and/or in vivo. For example, for in vivo introduction the fusion protein, the AAV vector, the AAV capsid, or the pharmaceutical composition can be injected into a specific tissue site (e.g., the locus where a therapeutic effect is desired) or administered systemically (e.g., administering an AAV vector targeted to a locus where a therapeutic effect is desired). In vitro introduction into a cell includes methods known in the art such as electroporation and lipofection.

As used herein, the terms “effective amount,” “therapeutically effective amount,” and a “sufficient amount” of, e.g., the fusion protein, the AAV vector, the AAV capsid, or the pharmaceutical composition disclosed herein refer to a quantity sufficient to, when administered to the subject, including a human, effect beneficial or desired results, including clinical results, and, as such, an “effective amount” or synonym thereto depends on the context in which it is being applied. In some aspects, a therapeutically effective amount of an agent (e.g., the fusion protein, the AAV vector, the AAV capsid, or the pharmaceutical composition disclosed herein) is an amount that results in a beneficial or desired result in a subject as compared to a control.

The amount of a given agent (e.g., the fusion protein, the AAV vector, the AAV capsid, or the pharmaceutical composition disclosed herein) will correspond to such an amount will vary depending upon various factors, such as the given agent, the pharmaceutical formulation, the route of administration, the type of disease or disorder, the identity of the subject (e.g., age, sex, and/or weight) or host being treated, and the like.

The term “prophylactically effective amount,” as used herein, includes the amount of an agent, (e.g., the fusion protein, the AAV vector, the AAV capsid, or the pharmaceutical composition disclosed herein) that, when administered to a subject having or predisposed to have a disease or disorder (e.g., a complement-mediated disorder), is sufficient to prevent, reduce the risk of, reduce the symptoms of, or ameliorate the disease or disorder or one or more symptoms of the disease or disorder. Ameliorating the disease or disorder includes slowing the course of the disease or disorder or reducing the severity of later-developing disease or disorder. The “prophylactically effective amount” can vary depending on the characteristics of the agent, e.g., the fusion protein, the AAV vector, the AAV capsid, or the pharmaceutical composition, how the agent is administered, the degree of risk of disease, and the history, age, weight, family history, genetic makeup, the types of preceding or concomitant treatments, if any, and other individual characteristics of the subject to be treated.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “transfection” refers to methods to introduce exogenous nucleic acids into a cell. Methods of transfection include, but are not limited to, chemical methods, physical treatments and cationic lipids or mixtures. The list of agents that can be transfected into a cell is large and includes, e.g., mRNA, siRNA, shRNA, sense and/or anti-sense sequences, DNA encoding one or more genes or coding sequences including those organized into an expression plasmid, e.g., a vector.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST.

The terms “administer,” “administering,” “administration,” and the like, as used herein, refer to methods that may be used to enable delivery of a drug, e.g., the fusion protein, the AAV vector, the AAV capsid, or the pharmaceutical composition disclosed herein, to the desired site of biological action. Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In some aspects, the term treating refers to reducing inflammation (e.g. inflammation caused by an inflammatory diseases such as glaucoma or AMD).

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications. As used herein, the phrase “subject in need thereof” includes subjects, such as mammalian subjects, that would benefit from administration of a therapeutic agent, e.g., the fusion protein, the AAV vector, the AAV capsid, or the pharmaceutical composition disclosed herein.

As used herein, the terms “derived from” or “derivative” refer to a component that is isolated from or made using a specified molecule, or information (e.g., a nucleic acid sequence) from the specified molecule. For example, a polynucleotide sequence that is derived from another polynucleotide sequence can include a polynucleotide sequence that is identical or substantially similar to the polynucleotide sequence it derives from. In the case of polynucleotides, the derived species can be obtained by, for example, naturally occurring mutagenesis, artificial directed mutagenesis, or artificial random mutagenesis. The mutagenesis used to derive polynucleotides can be intentionally directed or intentionally random, or a mixture of each. The mutagenesis of a polynucleotide to create a different polynucleotide derived from the first polynucleotide can be a random event (e.g., caused by polymerase infidelity) and the identification of the derived polynucleotide can be made by appropriate screening methods known in the art. In some aspects, a polynucleotide sequence that is derived from a first polynucleotide sequence has a sequence identity of at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to the first polynucleotide sequence, respectively, wherein the derived polynucleotide sequence retains the biological activity of the original polynucleotide.

As used herein, the term “functional fragment thereof” refers to a fragment or portion of a protein that is still capable of one or more functions associated with the full protein (e.g., stimulating, modulating, regulating, or modifying). In some aspects, a functional fragment can comprise a truncated protein.

As used herein, “truncated” refers to a molecule or sequence (e.g., protein, polypeptide, nucleic acid, etc.) that is shortened by removing one or more portions therein. The truncation(s) can be in any portion(s) throughout the molecule, e.g., not limited to the N or C terminal portions.

As used herein, the term “delivery vector” or “vector” refers to any vehicle for the cloning of and/or transfer of a nucleic acid into a host cell, such as a plasmid, phage, transposon, cosmid, chromosome, artificial chromosome, virus, virion, etc. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “delivery vector” or “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, the delivery vector is selected from the group consisting of a viral vector (e.g., an AAV vector), a plasmid, a lipid, a protein particle, a bacterial vector, and a lysosome.

Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years.

As used herein, the term “complement-mediated disorder” refers to any disorder in which complement activation is known or suspected of being a contributing and/or at least partially causative factor in at least some subjects suffering from the disorder, e.g., disorders in which complement activation results in tissue damage. Non-limiting examples of complement-mediated disorders include, e.g., (i) various disorders characterized by hemolysis or hemolytic anemia such as atypical hemolytic uremic syndrome, cold agglutinin disease, paroxysmal nocturnal hemoglobinuria, transfusion reactions; (ii) transplant rejection (e.g., hyperacute or acute transplant rejection) or transplant dysfunction; (iii) disorders involving ischemia/reperfusion injury such as trauma, surgery (e.g., aneurysm repair), myocardial infarction, ischemic stroke; (iv) disorders of the respiratory system such as asthma and chronic obstructive pulmonary disease (COPD); (v) arthritis, e.g., rheumatoid arthritis; (vi) ocular disorders such as age-related macular degeneration (AMD), diabetic retinopathy, glaucoma, and uveitis.

“Disorder” is used interchangeably herein with “disease”, “condition”, and similar words to refer to any impairment of health or state of abnormal functioning of an organism, e.g., any state in which medical and/or surgical management is indicated or for which a subject appropriately seeks medical and/or surgical attention. It should also be understood that the listing of a particular disorder within a particular category is for convenience and is not intended to limit the invention. It will be understood that certain disorders could appropriately be listed in multiple categories.

### II. Truncated CR1 Polypeptides

Certain aspects of the present disclosure provides a polypeptide comprising a truncated complement receptor 1 (CR1) protein. In some aspects, the truncated CR1 protein has an amino acid sequence with at least 85%, at least 90%, at least 95%, at least 96%, at least 97%, at least 98%, at least 99%, or 100% identity to any one of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In some aspects, the polypeptide comprises a truncated CR1 protein having an amino acid sequence with at least 85%, at least 90%, at least 95%, at least 96%, at least 97%, at least 98%, at least 99%, or 100% identity to SEQ ID NO: 3.

In some aspects, the polypeptide comprises a truncated CR1 protein having an amino acid sequence with at least 85%, at least 90%, at least 95%, at least 96%, at least 97%, at least 98%, at least 99%, or 100% identity to SEQ ID NO: 4.

In some aspects, the polypeptide comprises a truncated CR1 protein having an amino acid sequence with at least 85%, at least 90%, at least 95%, at least 96%, at least 97%, at least 98%, at least 99%, or 100% identity to SEQ ID NO: 5.

In some aspects, the polypeptide comprises a truncated CR1 protein having an amino acid sequence with at least 85%, at least 90%, at least 95%, at least 96%, at least 97%, at least 98%, at least 99%, or 100% identity to SEQ ID NO: 6.

In some aspects, the polypeptide comprises a truncated CR1 protein having an amino acid sequence with at least 85%, at least 90%, at least 95%, at least 96%, at least 97%, at least 98%, at least 99%, or 100% identity to SEQ ID NO: 7.

In some aspects, the polypeptide comprises a truncated CR1 protein having an amino acid sequence with at least 85%, at least 90%, at least 95%, at least 96%, at least 97%, at least 98%, at least 99%, or 100% identity to SEQ ID NO: 8.

In some aspects, the polypeptide comprises a truncated CR1 protein having an amino acid sequence with at least 85%, at least 90%, at least 95%, at least 96%, at least 97%, at least 98%, at least 99%, or 100% identity to SEQ ID NO: 9.

In some aspects, the polypeptide comprises a truncated CR1 protein having an amino acid sequence with at least 85%, at least 90%, at least 95%, at least 96%, at least 97%, at least 98%, at least 99%, or 100% identity to SEQ ID NO: 10.

In some aspects, the polypeptide comprises a truncated CR1 protein having an amino acid sequence with at least 85%, at least 90%, at least 95%, at least 96%, at least 97%, at least 98%, at least 99%, or 100% identity to SEQ ID NO: 11.

In some aspects, the polypeptide is a fusion protein.

In some aspects, fusion protein comprises a truncated CR1 protein disclosed herein and a complement receptor 2 (CR2) protein or a functional fragment thereof.

In some aspects, the fusion protein comprises a truncated CR1 protein disclosed herein and any one of the following: a vascular endothelial growth factor (VEGF) binding domain or functional fragment thereof (e.g., a VEGF inhibiting domain (VID)), an anti-C5 binding domain, a serum albumin-binding receptor, a granulocyte colony stimulating factor (G-CSF) binding domain or functional fragment thereof, a G-CSF receptor (G-CSFR) binding domain or functional fragment thereof, a Factor I binding domain or functional fragment thereof, a Factor II (prothrombin)/thrombin binding domain or functional fragment thereof, a Factor III binding domain or functional fragment thereof, a Factor V binding domain or functional fragment thereof, a Factor VII binding domain or functional fragment thereof, a Factor VIII binding domain or functional fragment thereof, a Factor IX binding domain or functional fragment thereof, a Factor X binding domain or functional fragment thereof, a Factor XI binding domain or functional fragment thereof, a Factor XII binding domain or functional fragment thereof, or a Factor XIII binding domain or functional fragment thereof.

In some aspects, the fusion protein comprises a CR1 protein or functional fragment thereof comprising Short Consensus Repeats (SCRs) 1-4, 7-11, and 14-17. In some aspects, the fusion protein comprises a CR1 protein or functional fragment thereof that consists essentially of SCRs 1-4, 7-11, and 14-17. In some aspects, the fusion protein comprises a CR1 protein or functional fragment thereof that does not contain SCRs 5-6, 12-13, and 18-30. In some aspects, the fusion protein comprises a CR1 protein or functional fragment thereof comprising SCRs 1-4, 7-11, and 14-17, and does not contain SCRs 5-6, 12-13, and 18-30.

In some aspects, the fusion protein comprises a CR1 protein or functional fragment thereof comprising SCRs 1-14. In some aspects, the fusion protein comprises a CR1 protein or functional fragment thereof that consists essentially of SCRs 1-14. In some aspects, the fusion protein comprises a CR1 protein or functional fragment thereof does not contain SCRs 15-30. In some aspects, the fusion protein comprises a CR1 protein or functional fragment thereof comprising SCRs 1-14, and does not contain SCRs 15-30.

In some aspects, the fusion protein comprises a CR1 protein or functional fragment thereof that comprises SCRs 1-3, 7-10, and 14-17. In some aspects, the fusion protein comprises a CR1 protein or functional fragment thereof that consists essentially of SCRs 1-3, 7-10, and 14-17. In some aspects, the fusion protein comprises a CR1 protein or functional fragment thereof that does not contain SCRs 5-6, 11-13, and 18-30. In some aspects, the fusion protein comprises a CR1 protein or functional fragment thereof comprising SCRs 1-3, 7-10, and 14-17, and does not contain SCRs 5-6, 11-13, and 18-30.

In some aspects, the fusion protein comprises a CR1 protein or functional fragment thereof comprising SCRs 1-17. In some aspects, the fusion protein comprises a CR1 protein or functional fragment thereof that consists essentially of SCRs 1-17. In some aspects, the fusion protein comprises a CR1 protein or functional fragment thereof that does not contain SCRs 18-30. In some aspects, the fusion protein comprises a CR1 protein or functional fragment thereof comprising SCRs 1-17, and does not contain SCRs 18-30.

### III. CR2-CR1 Fusion Proteins and Other Fusion Proteins

### Complement Receptor 2 (“CR2”)

Naturally occurring CR2 is a transmembrane protein expressed predominantly on mature B cells and follicular dendritic cells. CR2 is a member of the C3 binding protein family. Natural ligands for CR2 include, for example, iC3b, C3dg, and C3d, and cell-bound breakdown fragments of C3b that bind to the two N-terminal SCR domains of CR2. Other ligands include CD23, IFN-α, Epstein-Barr Virus, and various forms of DNA. C3b is the larger of two elements formed by the cleavage of complement component 3. C3a is the smaller element. Cleavage of C3 results initially in the generation of C3b and the covalent attachment of this C3b to the activating cell surface. The C3b fragment is involved in the generation of enzymatic complexes that amplify the complement cascade. On a cell surface, C3b is rapidly converted to inactive iC3b, particularly when deposited on a host surface containing regulators of complement activation (i.e., most host tissue). Even in absence of membrane bound complement regulators, substantial levels of iC3b are formed. iC3b is subsequently digested to the membrane bound fragments C3dg and then C3d by serum proteases, but this process is relatively slow. Thus, the C3 ligands for CR2 are relatively long lived once they are generated and will be present in high concentrations at sites of complement activation. CR2 therefore can serve as a potent targeting vehicle for bringing molecules to the site of complement activation.

Further, the complement cascade is a vital, highly regulated component of the immune system responsible for both innate defense against pathogens and adaptive immune response. Activation of each pathway (classical, lectin, and alternative) occurs via binding of a different factor, with the pathways converging at the complement component 3 (C3) cleavage step. C3 is an abundant central protein of the innate immune system that modulates numerous complement effector functions via different activation states. Activation of C3 occurs when membrane-bound C3 convertases (C4bC2b in the classical and lectin pathway and C3bBb in the alternative pathway) cleave soluble C3 into functional fragments C3a and C3b. Upon C3 cleavage, the released C3b binds to the cell surface and can form additional C3 convertase units allowing for further C3 cleavage, thus amplifying the complement cascade. Accumulation of C3b on the cell surface, also known as opsonization, is important in the immune response to promote phagocytosis. Within host cells, regulatory proteins including complement factors H and I (CFH, CFI) bind to cleavage product C3b, inactivating the system and preventing further cascading. Without them, multiple C3b molecules form C5 convertase complexes with other complement factors to cleave C5 into C5a and C5b. Within the complement cascade, C5a, C3b, and C3a regulate inflammatory responses at the site of complement activation. During the final steps of the complement cascade, C5b forms a membrane attack complex (MAC) with C6, C7, C8, and C9, which then binds to cell surfaces and creates pores that cause cell rupturing and death. Under normal conditions, the propagation of the complement cascade is regulated to prevent its overactivation and protect against lysis of healthy host cells.

The role of complement dysfunction in GA pathogenesis is supported and evidenced by both physiologic and genetic data. Complement factors C3, C5, CFH, and activated MAC have been found in drusen extracted from human donor eyes (Boyer, D S, et al., Retina 37(5):819-835(2017)). Elevated complement factors were similarly observed in Bruch's membrane and choriocapillaris of patients with advanced AMD as compared to individuals without large drusen (Loyet, K M, et al., Invest Ophthalmol Vis Sci. 53(10):6628-6637 (2012)). Systemically, plasma samples of patients with AMD and GA have increased levels of complement activation products, including C3d, C3a, Ba, Bb, and C5a (Scholl, H P N et al., PloS one 3(7):e2593 (2008); Reynolds, R, et al., Invest Opthalmol Vis Sci. 50(12): 5818-5827 (2009)). Genetically, complement involvement in GA is supported by genome-wide association studies which have identified a variant in the CFH gene that is associated with a high risk of AMD (Boyer, D S, et al., Retina 37(5):819-835(2017)). Further, across a study of over 17,000 advanced AMD cases compared to 60,000 controls, 19 other SNPs were identified as common AMD risk variants, with several being in genes involved in the complement cascade (Fritsche, L G, et al. Nat Genet 45(4):433-439 (2013)).

Naturally occurring CR2 contains an extracellular portion having 15 or 16 repeating units known as short consensus repeats (SCR domains). The SCR domains have a typical framework of highly conserved residues including four cysteines, two prolines, one tryptophan and several other partially conserved glycines and hydrophobic residues. In some aspects, SEQ ID NO: 62 represents the full-length human CR2 protein sequence (e.g., NCBI Accession Number NP_001006659.1). In some aspects, SEQ ID NO: 63 represents the full-length human CR2 protein sequence (e.g., NCBI Accession Number NP_001868.2). Amino acids 1-20 comprise the leader peptide, amino acids 23-82 comprise SCR1, amino acids 91-146 comprise SCR2, amino acids 154-210 comprise SCR3, and amino acids 215-271 comprise SCR4. The active site (C3d binding site) is located in SCR 1-2 (the first two N-terminal SCR domains). These SCR domains are separated by short sequences of variable length that serve as spacers. It is understood that species and strain variations exist for the disclosed peptides, polypeptides, and proteins, and that the CR2 or a functional fragment thereof described herein encompasses all species and strain variations.

### Complement Receptor 1 (“CR1”)

Naturally occurring CR1 is a ˜200 kDa glycoprotein encoded by the CR1 gene (on chromosome region 1q32). The extracellular domain of the most common form of CR1 is composed of a series of 30 short consensus repeats (SCRs). The SCRs are distributed in four long homologous repeats (LHRs A, B, C, and D), arising from duplication of a seven-SCR unit (See Klickstein, B Y L B, et al., J Exp. 165:1095-1112 (1987); Wong, W W, et al., J Exp Med. 169:847-863 (1989); Moulds, J M, et al., Blood 97:2879-2885 (2001)). CR1 is primarily expressed in erythrocytes, B and T cells, neutrophils, monocytes, and dendritic cells, as well as in neurons, microglia, and the choroid plexus of the brain, in the membrane or in soluble form (See Khera, R., et al., Mol Immunol. 46:761-772 (2009)). The soluble CR1 (sCR1) form results from proteolytic cleavage in terminal secretory vesicles or in the cell membrane (See Danielsson, C., et al., Eur J Immunol. 24:2725-2731 (1994); Hamer, I., et al., Biochem J 190:183-190 (1998)). Both CR1 forms act to regulate complement activity by binding cleaved C3b and C4b components as well as the complement cascade initiation molecules mannose-binding lectin (MBL-2), ficolins (FCN1, FCN2, and FCN3), and C1q deposited in altered cell components or pathogens. CR1 competes with serine proteases (MASPs) for the same binding sites on the collagenous tails of MBL and FCNs, inhibiting the initiation of the lectin pathway of complement (See Furtdao, P. B., et al., J Mol Biol. 375:102-118 (2008)). Upon binding, the membrane-bound form of CR1 internalizes the opsonized elements or presents them to other immune cells, preventing the formation of the C5 convertase. This blocks the formation of the MAC. CR1 might also prevent excessive complement activation, acting as a cofactor for the Factor I-mediated cleavage of soluble/bound C3b and C4b (See Liu, D., et al., Immunopharmacol Immunotoxicol. 31:524-535 (2009)). On the other hand, binding of opsonized elements fosters antigen presentation to lymphocytes, increasing antibody production, and the humoral response (Khera, R., 2009; Anna, S., Curr Top Microbiol Immunol. 408:67-87 (2017)).

In some aspects, SEQ ID NO: 64 represents the full-length human CR1 protein sequence (e.g., NCBI Accession Number NP_000564.2). In some aspects, SEQ ID NO: 65 represents the full-length human CR1 protein sequence (e.g., NCBI Accession Number NP_000642.3). In some aspects, SEQ ID NO: 66 represents the full-length human CR1 protein sequence (e.g., NCBI Accession Number NP_001368780.1). CR1 SCR1-3 contains a binding site for C4b and a weak binding site for C3b (“site 1”), which provides decay accelerating activity. CR1 SCR8-10 and SCR15-17 contain binding sites (“site 2”) for C3b and C4b, which provides cofactor activity and decay accelerating activity.

### CR2-CR1 Fusion Protein

In some aspects, provided herein is a fusion protein comprising a complement receptor 2 (CR2) protein or a functional fragment thereof, and a complement receptor 1 (CR1) protein or a functional fragment thereof. In some aspects, the CR2 protein or functional fragment thereof comprises an amino acid sequence having at least 85%, at least 90%, at least 95%, at least 96%, at least 97%, at least 98%, at least 99%, or 100% identity to SEQ ID NO: 1 or SEQ ID NO: 2. In some aspects, the CR1 protein or functional fragment thereof (e.g., a truncated CR1 protein) comprises an amino acid sequence having at least 85%, at least 90%, at least 95%, at least 96%, at least 97%, at least 98%, at least 99%, or 100% identity to any of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In some aspects, the C terminus of the complement receptor 2 protein or functional fragment thereof is linked to the N terminus of the complement receptor 1 protein or functional fragment thereof by a first linker. In some aspects, the short consensus repeats (SCRs) of the CR1 protein or functional fragment is linked by a second linker (also referred to herein as a spacer).

In some aspects, the first and/or second linker (or spacer) is a (GGGGS)n linker, wherein n is between 2 and 12 (e.g., (GGGGS)₁, (GGGGS)₂, (GGGGS)₃, (GGGGS)₄, (GGGGS)₅, (GGGGS)₆, (GGGGS)₇, (GGGGS)₈, (GGGGS)₉, (GGGGS)₁₀, (GGGGS)₁₁, or (GGGGS)₁₂). In some aspects, the first and/or second linker (or spacer) comprises an amino acid sequence of any one of SEQ ID NOs: 139 and 156-160.

In some aspects, the CR2 portion or a functional fragment thereof comprises one or more of SCR1, SCR2, SCR3, SCR4, SCR5, SCR6, SCR7, SCR8, SCR9, SCR10, SCR11, SCR12, SCR13, SCR14, or SCR15 of the CR2 protein. In some aspects, the CR2 portion or functional fragment thereof comprises SCR1 of the CR2 protein. In some aspects, the CR2 portion or functional fragment thereof comprises SCR2 of the CR2 protein. In some aspects, the CR2 portion or functional fragment thereof comprises SCR3 of the CR2 protein. In some aspects, the CR2 portion or functional fragment thereof comprises SCR4 of the CR2 protein. In some aspects, the CR2 portion or functional fragment thereof comprises SCR5 of the CR2 protein. In some aspects, the CR2 portion or functional fragment thereof comprises SCR6 of the CR2 protein. In some aspects, the CR2 portion or functional fragment thereof comprises SCR7 of the CR2 protein. In some aspects, the CR2 portion or functional fragment thereof comprises SCR8 of the CR2 protein. In some aspects, the CR2 portion or functional fragment thereof comprises SCR9 of the CR2 protein. In some aspects, the CR2 portion or functional fragment thereof comprises SCR10 of the CR2 protein. In some aspects, the CR2 portion or functional fragment thereof comprises SCR11 of the CR2 protein. In some aspects, the CR2 portion or functional fragment thereof comprises SCR12 of the CR2 protein. In some aspects, the CR2 portion or functional fragment thereof comprises SCR13 of the CR2 protein. In some aspects, the CR2 portion or functional fragment thereof comprises SCR14 of the CR2 protein. In some aspects, the CR2 portion or functional fragment thereof comprises SCR15 of the CR2 protein. In some aspects, the SCR sequences correspond to the SCRs of CR2 protein having a sequence of SEQ ID NO: 62 or SEQ ID NO: 63.

In some aspects, the CR2 portion or functional fragment thereof comprises SCR1 and SCR2 of the CR2 protein (e.g., SEQ ID NO: 2). In some aspects, the CR2 portion or functional fragment thereof comprises SCR1 to SCR3 of the CR2 protein. In some aspects, the CR2 portion or functional fragment thereof comprises SCR1 to SCR4 of the CR2 protein (e.g., SEQ ID NO: 1). In some aspects, the CR2 portion or functional fragment thereof comprises SCR1 to SCR5 of the CR2 protein. In some aspects, the CR2 portion or functional fragment thereof comprises SCR1 to SCR6 of the CR2 protein. In some aspects, the CR2 portion or functional fragment thereof comprises SCR1 to SCR6 of the CR2 protein. In some aspects, the CR2 portion or functional fragment thereof comprises SCR1 to SCR7 of the CR2 protein. In some aspects, the CR2 portion or functional fragment thereof comprises SCR1 to SCR8 of the CR2 protein. In some aspects, the CR2 portion or functional fragment thereof comprises SCR1 to SCR9 of the CR2 protein. In some aspects, the CR2 portion or functional fragment thereof comprises SCR1 to SCR10 of the CR2 protein. In some aspects, the CR2 portion or functional fragment thereof comprises SCR1 to SCR11 of the CR2 protein. In some aspects, the CR2 portion or functional fragment thereof comprises SCR1 to SCR12 of the CR2 protein. In some aspects, the CR2 portion or functional fragment thereof comprises SCR1 to SCR13 of the CR2 protein. In some aspects, the CR2 portion or functional fragment thereof comprises SCR1 to SCR14 of the CR2 protein. In some aspects, the CR2 portion or functional fragment thereof comprises SCR1 to SCR15 of the CR2 protein.

In some aspects, the CR2 portion or functional fragment thereof comprises at least SCR1 and SCR2 (e.g., SEQ ID NO: 2). In some aspects, the CR2 portion or functional fragment thereof comprises at least SCR1, SCR2, SCR3, and SCR4 (e.g., SEQ ID NO: 1). In some aspects, the CR2 portion or functional fragment thereof comprises at least two or more of SCR1, SCR2, SCR3, SCR4, SCR5, SCR6, SCR7, SCR8, SCR9, SCR10, SCR11, SCR12, SCR13, SCR14, SCR15 of the CR2 protein.

In some aspects, the CR2 portion or functional fragment thereof consists essentially of CR2 SCRs 1-4. In some aspects, the CR2 portion or functional fragment thereof comprises CR2 SCRs 1-4 and does not contain SCRs 5-15.

In some aspects, the CR2 portion or functional fragment thereof consists essentially of CR2 SCRs 1-2. In some aspects, the CR2 portion or functional fragment thereof comprises CR2 SCRs 1-2 and does not contain SCRs 3-15.

In some aspects, the CR1 portion or functional fragment thereof comprises one or more of SCR1, SCR2, SCR3, SCR4, SCR5, SCR6, SCR7, SCR8, SCR9, SCR10, SCR11, SCR12, SCR13, SCR14, SCR15, SCR16, SCR17, SCR18, SCR19, SCR20, SCR21, SCR22, SCR23, SCR24, SCR25, SCR26, SCR27, SCR28, SCR29, or SCR30 of the CR1 protein. In some aspects, the CR1 portion or functional fragment thereof comprises SCR1 of the CR1 protein. In some aspects, the CR1 portion or functional fragment thereof comprises SCR2 of the CR1 protein. In some aspects, the CR1 portion or functional fragment thereof comprises SCR3 of the CR1 protein. In some aspects, the CR1 portion or functional fragment thereof comprises SCR4 of the CR1 protein. In some aspects, the CR1 portion or functional fragment thereof comprises SCR5 of the CR1 protein. In some aspects, the CR1 portion or functional fragment thereof comprises SCR6 of the CR1 protein. In some aspects, the CR1 portion or functional fragment thereof comprises SCR7 of the CR1 protein. In some aspects, the CR1 portion or functional fragment thereof comprises SCR8 of the CR1 protein. In some aspects, the CR1 portion or functional fragment thereof comprises SCR9 of the CR1 protein. In some aspects, the CR1 portion or functional fragment thereof comprises SCR10 of the CR1 protein. In some aspects, the CR1 portion or functional fragment thereof comprises SCR11 of the CR1 protein. In some aspects, the CR1 portion or functional fragment thereof comprises SCR12 of the CR1 protein. In some aspects, the CR1 portion or functional fragment thereof comprises SCR13 of the CR1 protein. In some aspects, the CR1 portion or functional fragment thereof comprises SCR14 of the CR1 protein. In some aspects, the CR1 portion or functional fragment thereof comprises SCR15 of the CR1 protein. In some aspects, the CR1 portion or functional fragment thereof comprises SCR16 of the CR1 protein. In some aspects, the CR1 portion or functional fragment thereof comprises SCR17 of the CR1 protein. In some aspects, the CR1 portion or functional fragment thereof comprises SCR18 of the CR1 protein. In some aspects, the CR1 portion or functional fragment thereof comprises SCR19 of the CR1 protein. In some aspects, the CR1 portion or functional fragment thereof comprises SCR20 of the CR1 protein. In some aspects, the CR1 portion or functional fragment thereof comprises SCR21 of the CR1 protein. In some aspects, the CR1 portion or functional fragment thereof comprises SCR22 of the CR1 protein. In some aspects, the CR1 portion or functional fragment thereof comprises SCR23 of the CR1 protein. In some aspects, the CR1 portion or functional fragment thereof comprises SCR24 of the CR1 protein. In some aspects, the CR1 portion or functional fragment thereof comprises SCR25 of the CR1 protein. In some aspects, the CR1 portion or functional fragment thereof comprises SCR26 of the CR1 protein. In some aspects, the CR1 portion or functional fragment thereof comprises SCR27 of the CR1 protein. In some aspects, the CR1 portion or functional fragment thereof comprises SCR28 of the CR1 protein. In some aspects, the CR1 portion or functional fragment thereof comprises SCR29 of the CR1 protein. In some aspects, the CR1 portion or functional fragment thereof comprises SCR30 of the CR1 protein. In some aspects, the SCR sequences correspond to the SCRs of CR1 protein having a sequence of SEQ ID NO: 64, SEQ ID NO: 65 or SEQ ID NO: 66.

In some aspects, the CR1 portion or functional fragment thereof comprises SCR1 to SCR3 of the CR1 protein. In some aspects, the CR1 portion or functional fragment thereof comprises SCR8 to SCR10 of the CR1 protein. In some aspects, the CR1 portion or functional fragment thereof comprises SCR15 to SCR17 of the CR1 protein. In some aspects, the CR1 portion or functional fragment thereof comprises SCR1 to SCR3 and SCR8 to SCR10 of the CR1 protein. In some aspects, the CR1 portion or functional fragment thereof comprises SCR1 to SCR3 and SCR15 to SCR17 of the CR1 protein. In some aspects, the CR1 portion or functional fragment thereof comprises SCR1 to SCR3, SCR8 to SCR10, and SCR15 to SCR17 of the CR1 protein (e.g., SEQ ID NO: 9).

In some aspects, the CR1 portion or functional fragment thereof comprises at least three or more of SCR1, SCR2, SCR3, SCR4, SCR5, SCR6, SCR7, SCR8, SCR9, SCR10, SCR11, SCR12, SCR13, SCR14, SCR15, SCR16, SCR17, SCR18, SCR19, SCR20, SCR21, SCR22, SCR23, SCR24, SCR25, SCR26, SCR27, SCR28, SCR29, SCR30 of the CR1 protein.

In some aspects, the CR1 portion or functional fragment thereof comprises SCRs 1-4, 7-11, and 14-17. In some aspects, the CR1 portion or functional fragment thereof consists essentially of SCRs 1-4, 7-11, and 14-17.

In some aspects, the CR1 portion or functional fragment thereof does not contain SCRs 5-6, 12-13, and 18-30.

In some aspects, the CR1 portion or functional fragment thereof comprises SCRs 1-4, 7-11, and 14-17, and does not contain SCRs 5-6, 12-13, and 18-30.

In some aspects, the CR1 portion or functional fragment thereof comprises SCRs 1-14. In some aspects, the CR1 portion or functional fragment thereof consists essentially of SCRs 1-14.

In some aspects, the CR1 portion or functional fragment thereof does not contain SCRs 15-30.

In some aspects, the CR1 portion or functional fragment thereof comprises SCRs 1-14, and does not contain SCRs 15-30.

In some aspects, the CR1 portion or functional fragment thereof comprises SCRs 1-3, 7-10, and 14-17. In some aspects, the CR1 portion or functional fragment thereof consists essentially of SCRs 1-3, 7-10, and 14-17.

In some aspects, the CR1 portion or functional fragment thereof does not contain SCRs 5-6, 11-13, and 18-30.

In some aspects, the CR1 portion or functional fragment thereof comprises SCRs 1-3, 7-10, and 14-17 and does not contain SCRs 5-6, 11-13, and 18-30.

In some aspects, the CR1 portion or functional fragment thereof comprises SCRs 1-17. In some aspects, the CR1 portion or functional fragment thereof consists essentially of SCRs 1-17.

In some aspects, the CR1 portion or functional fragment thereof does not contain SCRs 18-30.

In some aspects, the CR1 portion or functional fragment thereof comprises SCRs 1-17 and does not contain SCRs 18-30.

In some aspects, the complement receptor 2 protein or functional fragment thereof comprises an amino acid sequence having at least 85%, at least 90%, at least 95%, at least 96%, at least 97%, at least 98%, at least 99%, or 100% identity to SEQ ID NO: 1. In some aspects, the complement receptor 2 protein or functional fragment thereof comprises an amino acid sequence having at least about 85%, at least about 90%, at least about 95%, at least about 96%, at least about 97%, at least about 98%, at least about 99%, or 100% identity to SEQ ID NO: 1.

In some aspects, the complement receptor 2 protein or functional fragment thereof comprises an amino acid sequence having at least 85%, at least 90%, at least 95%, at least 96%, at least 97%, at least 98%, at least 99%, or 100% identity to SEQ ID NO: 2. In some aspects, the complement receptor 2 protein or functional fragment thereof comprises an amino acid sequence having at least about 85%, at least about 90%, at least about 95%, at least about 96%, at least about 97%, at least about 98%, at least about 99%, or 100% identity to SEQ ID NO: 2.

In some aspects, the complement receptor 1 protein or functional fragment thereof (e.g., truncated CR1 protein) comprises an amino acid sequence having at least 85%, at least 90%, at least 95%, at least 96%, at least 97%, at least 98%, at least 99%, or 100% identity to SEQ ID NO: 3. In some aspects, the complement receptor 1 protein or functional fragment thereof (e.g., truncated CR1 protein) comprises an amino acid sequence having at least about 85%, at least about 90%, at least about 95%, at least about 96%, at least about 97%, at least about 98%, at least about 99%, or 100% identity to SEQ ID NO: 3.

In some aspects, the complement receptor 1 protein or functional fragment thereof (e.g., truncated CR1 protein) comprises an amino acid sequence having at least 85%, at least 90%, at least 95%, at least 96%, at least 97%, at least 98%, at least 99%, or 100% identity to SEQ ID NO: 4. In some aspects, the complement receptor 1 protein or functional fragment thereof (e.g., truncated CR1 protein) comprises an amino acid sequence having at least about 85%, at least about 90%, at least about 95%, at least about 96%, at least about 97%, at least about 98%, at least about 99%, or 100% identity to SEQ ID NO: 4.

In some aspects, the complement receptor 1 protein or functional fragment thereof (e.g., truncated CR1 protein) comprises an amino acid sequence having at least 85%, at least 90%, at least 95%, at least 96%, at least 97%, at least 98%, at least 99%, or 100% identity to SEQ ID NO: 5. In some aspects, the complement receptor 1 protein or functional fragment thereof (e.g., truncated CR1 protein) comprises an amino acid sequence having at least about 85%, at least about 90%, at least about 95%, at least about 96%, at least about 97%, at least about 98%, at least about 99%, or 100% identity to SEQ ID NO: 5.

In some aspects, the complement receptor 1 protein or functional fragment thereof (e.g., truncated CR1 protein) comprises an amino acid sequence having at least 85%, at least 90%, at least 95%, at least 96%, at least 97%, at least 98%, at least 99%, or 100% identity to SEQ ID NO: 6. In some aspects, the complement receptor 1 protein or functional fragment thereof (e.g., truncated CR1 protein) comprises an amino acid sequence having at least about 85%, at least about 90%, at least about 95%, at least about 96%, at least about 97%, at least about 98%, at least about 99%, or 100% identity to SEQ ID NO: 6.

In some aspects, the complement receptor 1 protein or functional fragment thereof (e.g., truncated CR1 protein) comprises an amino acid sequence having at least 85%, at least 90%, at least 95%, at least 96%, at least 97%, at least 98%, at least 99%, or 100% identity to SEQ ID NO: 7. In some aspects, the complement receptor 1 protein or functional fragment thereof (e.g., truncated CR1 protein) comprises an amino acid sequence having at least about 85%, at least about 90%, at least about 95%, at least about 96%, at least about 97%, at least about 98%, at least about 99%, or 100% identity to SEQ ID NO: 7.

In some aspects, the complement receptor 1 protein or functional fragment thereof (e.g., truncated CR1 protein) comprises an amino acid sequence having at least 85%, at least 90%, at least 95%, at least 96%, at least 97%, at least 98%, at least 99%, or 100% identity to SEQ ID NO: 8. In some aspects, the complement receptor 1 protein or functional fragment thereof (e.g., truncated CR1 protein) comprises an amino acid sequence having at least about 85%, at least about 90%, at least about 95%, at least about 96%, at least about 97%, at least about 98%, at least about 99%, or 100% identity to SEQ ID NO: 8.

In some aspects, the complement receptor 1 protein or functional fragment thereof (e.g., truncated CR1 protein) comprises an amino acid sequence having at least 85%, at least 90%, at least 95%, at least 96%, at least 97%, at least 98%, at least 99%, or 100% identity to SEQ ID NO: 9. In some aspects, the complement receptor 1 protein or functional fragment thereof (e.g., truncated CR1 protein) comprises an amino acid sequence having at least about 85%, at least about 90%, at least about 95%, at least about 96%, at least about 97%, at least about 98%, at least about 99%, or 100% identity to SEQ ID NO: 9.

In some aspects, the complement receptor 1 protein or functional fragment thereof (e.g., truncated CR1 protein) comprises an amino acid sequence having at least 85%, at least 90%, at least 95%, at least 96%, at least 97%, at least 98%, at least 99%, or 100% identity to SEQ ID NO: 10. In some aspects, the complement receptor 1 protein or functional fragment thereof (e.g., truncated CR1 protein) comprises an amino acid sequence having at least about 85%, at least about 90%, at least about 95%, at least about 96%, at least about 97%, at least about 98%, at least about 99%, or 100% identity to SEQ ID NO: 10.

In some aspects, the complement receptor 1 protein or functional fragment thereof (e.g., truncated CR1 protein) comprises an amino acid sequence having at least 85%, at least 90%, at least 95%, at least 96%, at least 97%, at least 98%, at least 99%, or 100% identity to SEQ ID NO: 11. In some aspects, the complement receptor 1 protein or functional fragment thereof (e.g., truncated CR1 protein) comprises an amino acid sequence having at least about 85%, at least about 90%, at least about 95%, at least about 96%, at least about 97%, at least about 98%, at least about 99%, or 100% identity to SEQ ID NO: 11.

In some aspects, the fusion protein comprises an amino acid sequence having at least 85%, at least 90%, at least 95%, at least 96%, at least 97%, at least 98%, at least 99%, or 100% identity to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some aspects, the fusion protein comprises an amino acid sequence having at least 85%, at least 90%, at least 95%, at least 96%, at least 97%, at least 98%, at least 99%, or 100% identity to SEQ ID NO: 12. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 12.

In some aspects, the fusion protein comprises an amino acid sequence having at least 85%, at least 90%, at least 95%, at least 96%, at least 97%, at least 98%, at least 99%, or 100% identity to SEQ ID NO: 13. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 13.

In some aspects, the fusion protein comprises an amino acid sequence having at least 85%, at least 90%, at least 95%, at least 96%, at least 97%, at least 98%, at least 99%, or 100% identity to SEQ ID NO: 14. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 14.

In some aspects, the fusion protein comprises an amino acid sequence having at least 85%, at least 90%, at least 95%, at least 96%, at least 97%, at least 98%, at least 99%, or 100% identity to SEQ ID NO: 15. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 15.

In some aspects, the fusion protein comprises an amino acid sequence having at least 85%, at least 90%, at least 95%, at least 96%, at least 97%, at least 98%, at least 99%, or 100% identity to SEQ ID NO: 16. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 16.

In some aspects, the fusion protein comprises an amino acid sequence having at least 85%, at least 90%, at least 95%, at least 96%, at least 97%, at least 98%, at least 99%, or 100% identity to SEQ ID NO: 17. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 17.

In some aspects, the fusion protein comprises an amino acid sequence having at least 85%, at least 90%, at least 95%, at least 96%, at least 97%, at least 98%, at least 99%, or 100% identity to SEQ ID NO: 18. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 18.

In some aspects, the fusion protein comprises an amino acid sequence having at least 85%, at least 90%, at least 95%, at least 96%, at least 97%, at least 98%, at least 99%, or 100% identity to SEQ ID NO: 19. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 19.

In some aspects, the fusion protein comprises an amino acid sequence having at least 85%, at least 90%, at least 95%, at least 96%, at least 97%, at least 98%, at least 99%, or 100% identity to SEQ ID NO: 20. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 20.

In some aspects, the fusion protein comprises an amino acid sequence having at least 85%, at least 90%, at least 95%, at least 96%, at least 97%, at least 98%, at least 99%, or 100% identity to SEQ ID NO: 21. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 21.

In some aspects, the fusion protein comprises an amino acid sequence having at least 85%, at least 90%, at least 95%, at least 96%, at least 97%, at least 98%, at least 99%, or 100% identity to SEQ ID NO: 22. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 22. In some aspects, the fusion protein corresponds to the TT32 CR2-CR1 fusion protein, which is a targeted inhibitor of the classical and alternative pathway C3 convertases. TT32 is a CR2-CR1 fusion protein using the first ten SCRs of CR1. (see, e.g., Fridkis-Hareli, Masha et al., Molecular immunology vol. 105:150-164 (2019); doi:10.1016/j.molimm.2018.09.013.

In some aspects, the fusion protein comprises an amino acid sequence having at least 85%, at least 90%, at least 95%, at least 96%, at least 97%, at least 98%, at least 99%, or 100% identity to SEQ ID NO: 23. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 23.

In some aspects, the fusion protein comprises an amino acid sequence having at least 85%, at least 90%, at least 95%, at least 96%, at least 97%, at least 98%, at least 99%, or 100% identity to SEQ ID NO: 24. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 24.

In some aspects, the fusion protein comprises an amino acid sequence having at least 85%, at least 90%, at least 95%, at least 96%, at least 97%, at least 98%, at least 99%, or 100% identity to SEQ ID NO: 25. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 25.

In some aspects, the fusion protein comprises an amino acid sequence having at least 85%, at least 90%, at least 95%, at least 96%, at least 97%, at least 98%, at least 99%, or 100% identity to SEQ ID NO: 26. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 26.

In some aspects, the fusion protein comprises an amino acid sequence having at least 85%, at least 90%, at least 95%, at least 96%, at least 97%, at least 98%, at least 99%, or 100% identity to SEQ ID NO: 27. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 27.

In some aspects, the fusion protein comprises an amino acid sequence having at least 85%, at least 90%, at least 95%, at least 96%, at least 97%, at least 98%, at least 99%, or 100% identity to SEQ ID NO: 28. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 28.

In some aspects, the present disclosure provides a nucleic acid encoding any of CR2 portions, CR1 portions, or the fusion proteins disclosed herein.

In some aspects, provided herein is a polynucleotide comprising a promoter operably linked to a nucleic acid encoding a CR2-CR1 fusion protein. In some aspects, the polynucleotide comprises a nucleic acid sequence having at least 85%, at least 90%, at least 95%, at least 96%, at least 97%, at least 98%, at least 99%, or 100% identity to any of SEQ ID NOs: 29-61.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to any of SEQ ID NOs: 77-109.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to any of SEQ ID NOs: 110-113.

In some aspects, the nucleic acid encoding a CR1 polypeptide comprises a nucleic acid sequence having at least 85%, at least 90%, at least 95%, at least 96%, at least 97%, at least 98%, at least 99%, or 100% identity to any of SEQ ID NOs: 114-135.

In some aspects, the polynucleotide comprises a nucleic acid sequence having at least 85%, at least 90%, at least 95%, at least 96%, at least 97%, at least 98%, at least 99%, or 100% identity to any of SEQ ID NOs: 141 or 142.

In some aspects, the present disclosure provides a plasmid comprising a nucleic acid encoding any of the fusion proteins disclosed herein.

In some aspects, the fusion protein further comprises a human opticin signaling peptide (e.g., SEQ ID NO: 67).

In some aspects, suitable 3′ untranslated sequence can also be operably linked to the modified nucleic acid sequences encoding any of the fusion proteins disclosed herein. Suitable 3′ untranslated regions can be those naturally associated with the nucleotide sequence or can be derived from different genes, such as for example the bovine growth hormone 3′ untranslated region (e.g., bGH polyadenylation signal, SV40 polyadenylation signal, SV40 polyadenylation signal and enhancer sequence).

In some aspects, additional nucleotide sequences can be operably linked to the modified nucleic acid sequence(s) encoding a fusion protein disclosed herein, such as nucleotide sequences encoding signal sequences, nuclear localization signals, expression enhancers, and the like.

In some aspects, the nucleic acid disclosed herein further comprises an Internal Ribosome Entry Site (IRES) (e.g., SEQ ID NO: 70).

### Other CR1 Fusion Proteins

In some aspects, provided herein is a polypeptide comprising a complement receptor 1 (CR1) protein or functional fragment thereof. In some aspects, the complement receptor 1 protein or functional fragment thereof comprises an amino acid sequence having at least 85%, at least 90%, at least 95%, at least 96%, at least 97%, at least 98%, at least 99%, or 100% identity to any of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In some aspects, the polypeptide further comprises a second domain.

In some aspects, the second domain comprises a vascular endothelial growth factor (VEGF) binding domain or functional fragment thereof (e.g., a VEGF inhibiting domain (VID)), an anti-C5 binding domain, a serum albumin-binding receptor, a granulocyte colony stimulating factor (G-CSF) binding domain or functional fragment thereof, a G-CSF receptor (G-CSFR) binding domain or functional fragment thereof, a Factor I binding domain or functional fragment thereof, a Factor II (prothrombin)/thrombin binding domain or functional fragment thereof, a Factor III binding domain or functional fragment thereof, a Factor V binding domain or functional fragment thereof, a Factor VII binding domain or functional fragment thereof, a Factor VIII binding domain or functional fragment thereof, a Factor IX binding domain or functional fragment thereof, a Factor X binding domain or functional fragment thereof, a Factor XI binding domain or functional fragment thereof, a Factor XII binding domain or functional fragment thereof, or a Factor XIII binding domain or functional fragment thereof. In some aspects, the binding domain is an antibody or antigen-binding fragment thereof. In some aspects, the antigen-binding fragment thereof comprises a Fab, Fab, F(ab′)2, single chain Fv(scFv), disulfide linked Fv, V-NAR domain, IgNar, intrabody, IgGL1CH2, minibody, F(ab′)3, tetrabody, triabody, diabody, single domain antibody, DVD-Ig, Fcab, mAb2, (scFv)2, or scFv-Fc.

Vascular endothelial growth factor (VEGF) is one of the man important protein for promoting angiogenesis, which is a tightly regulated process of developing new blood vessels from a pre-existing vascular network (Ferrara, N., (2004), Endocrine Reviews, 25(4): 581-611). Angiogenesis is required during development and normal physiological processes such as wound healing, and is also involved in a number of disease pathogenesis, including AMD, RA, Diabetic Retinopathy, tumor growth and metastasis. Inhibition of angiogenesis has been shown to be effective in therapeutic applications.

In some aspects, the fusion protein comprises the truncated CR1 protein and a VEGF inhibiting domain (VID). In some aspects, the VID-CR1 fusion protein further comprises a half-life prolonging domain (e.g., an immunoglobulin Fc region). In some aspects, the half-life prolonging domain is an IgG1 Fc domain or fragment thereof comprising an amino acid sequence having at least 85%, at least 90%, at least 95%, at least 96%, at least 97%, at least 98%, at least 99%, or 100% identity to any of SEQ ID NO: 138 or 143-145.

In some aspects, provided herein is a fusion protein comprising a vascular endothelial growth factor (VEGF) binding domain or functional fragment thereof, optionally an IgG1 Fc domain or fragment thereof, and a complement receptor 1 (CR1) protein or functional fragment thereof. In some aspects, the VEGF binding domain or functional fragment thereof comprises an amino acid sequence having at least 85%, at least 90%, at least 95%, at least 96%, at least 97%, at least 98%, at least 99%, or 100% identity to any of SEQ ID NO: 136, 137, 146, or 147. In some aspects, the VEGF binding domain or functional fragment thereof comprises i) an amino acid sequence having at least 85%, at least 90%, at least 95%, at least 96%, at least 97%, at least 98%, at least 99%, or 100% identity to SEQ ID NO: 136 and i) an amino acid sequence having at least 85%, at least 90%, at least 95%, at least 96%, at least 97%, at least 98%, at least 99%, or 100% identity to SEQ ID NO: 137. In some aspects, the IgG1 Fc domain or fragment thereof comprises an amino acid sequence having at least 85%, at least 90%, at least 95%, at least 96%, at least 97%, at least 98%, at least 99%, or 100% identity to any of SEQ ID NO: 138.

In some aspects, the complement receptor 1 protein or functional fragment thereof of the fusion protein comprises an amino acid sequence having at least 85%, at least 90%, at least 95%, at least 96%, at least 97%, at least 98%, at least 99%, or 100% identity to any of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In some aspects, the fusion protein further comprises a linker having an amino acid sequence corresponding to SEQ ID NO: 139.

In some aspects, the fusion protein comprises an amino acid sequence having at least 85%, at least 90%, at least 95%, at least 96%, at least 97%, at least 98%, at least 99%, or 100% identity to SEQ ID NO: 140.

In some aspects, the C terminus of the VID is linked to the N terminus of the CR1 protein or functional fragment thereof by a linker. In some aspects, the C terminus of the CR1 protein or functional fragment thereof is linked to the N terminus of the VID by a linker. In some aspects, the fusion protein comprises, from the N-terminal to C-terminal, a VEGF inhibiting domain (VID), optionally an immunoglobulin Fc region, optionally a linker, and a truncated CR1 protein, wherein the fusion protein inhibits or reduces complement pathway activation and VEGF activity.

In some aspects, provided herein is a nucleic acid encoding a VID-CR1 fusion protein, wherein the CR1 portion of the VID-CR1 fusion protein has an amino acid sequence with at least 85%, at least 90%, at least 95%, at least 96%, at least 97%, at least 98%, at least 99%, or 100% identity to any of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In some aspects, the VID of the VID-CR1 fusion protein has an amino acid sequence with at least 85%, at least 90%, at least 95%, at least 96%, at least 97%, at least 98%, at least 99%, or 100% identity to any of SEQ ID NO: 136, 137, 146, or 147.

In some aspects, the VID-CR1 fusion protein further comprises a linker having an amino acid sequence with at least 95%, at least 96%, at least 97%, at least 98%, at least 99%, or 100% identity to SEQ ID NO: 139.

In some aspects, the fusion protein comprises an amino acid sequence having at least 85%, at least 90%, at least 95%, at least 96%, at least 97%, at least 98%, at least 99%, or 100% identity to SEQ ID NO: 140.

In some aspects, provided herein is a vector for delivering any of the nucleic acids disclosed herein to a subject, wherein the vector is a viral vector or a non-viral vector.

In some aspects, the viral vector is selected from the group consisting of an adenoviral vector, an adeno-associated virus (AAV) vector, or a lentiviral vector.

In some aspects, provided herein is an adeno-associated virus (AAV) vector comprising a polynucleotide comprising a promoter operably linked to a nucleic acid encoding a VID-CR1 fusion protein disclosed herein.

### Half-Life Prolonging Domain

The present disclosure provides a half-life prolonging domain that can be a component of any fusion protein disclosed herein. For example, Fc regions from an immunoglobulin can be incorporated into a fusion polypeptide to increase half-life in vivo. A half-life prolonging domain can comprise an Fc region from any immunoglobulin isotype, subclass, or allotype. In some aspects, the half-life prolonging domain is an Fc region from an immunoglobulin isotype selected from the group consisting of IgG, IgA, IgD, IgM, and IgE. In some aspects, the half-life prolonging domain comprises an immunoglobulin Fc region. In some aspects, the Fc region is a human Fc of IgG1, IgG2, IgG3 or IgG4. In some aspects, the Fc region is a human Fc of IgA1 or IgA2. In some aspects, the Fc region is a human Fc of IgD. In some aspects, the Fc region is a human Fc of IgE. In some aspects, the Fc region is a human Fc of IgM. In some aspects, the Fc region is glycosylated. In some aspects, the Fc region comprises the amino acid sequence selected from the group consisting of SEQ ID NOs: 138 or 143-145. In some aspects, the half-life prolonging domain can be a polypeptide or fragment thereof selected from the group consisting of an antibody, albumin, or protease inhibitor (e.g., alpha 1-antitrypsin). In some aspects, the half-life prolonging domain can be an amino acid sequence selected from the group consisting of a glycine-rich amino acid sequence, PESTAG sequence, or PAS sequence. The half-life prolonging domain can be any polypeptide or amino acid sequence known in the art to increase the half-life of a polypeptide in vivo. See Kontermann, R. (Ed.) (2011). Therapeutic Proteins: Strategies to Modulate their Plasma Half-lives, which is incorporated herein by reference in its entirety. In some aspects, the half-life prolonging domain is from a mammal, such as a human, baboon, chimpanzee, mouse, or rat.

In some aspects, amino acid sequence variants of the half-life prolonging domains provided herein are contemplated. For example, it may be desirable to improve the biological properties of the half-life prolonging domain. Amino acid sequence variants of a half-life prolonging domain may be prepared by introducing appropriate modifications into the nucleotide sequence encoding the half-life prolonging domain, or by peptide synthesis. Such modifications include, for example, deletions from, and/or insertions into and/or substitutions of residues within the amino acid sequences of the half-life prolonging domain. Any combination of deletion, insertion, and substitution can be made to arrive at the final construct, provided that the final construct possesses the desired characteristics, e.g., prolonging half-life of the fusion protein. Provided herein are variants of a half-life prolonging domain that can be a component of any fusion polypeptide disclosed herein. In some aspects, the half-life prolonging domain variant is an Fc region variant. Variants of the Fc region are known in the art, for example U.S. Patent Application Publication No. 2010/02493852, and U.S. patent application publication number 2006/01341105, which are incorporated herein by reference in their entirety. In some aspects, one or more amino acid modifications may be introduced into the Fc region of a fusion polypeptide provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions. In some aspects, the Fc region comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 138 or 143-145. In some aspects, the Fc region variant is glycosylated.

### IV. Delivery Vectors

In some aspects, the fusion protein is administered via direct injection. In some aspects, the fusion protein is delivered via a delivery vector comprising a nucleic acid encoding the fusion protein.

In some aspects, the delivery vector is a viral vector, a non-viral vector, a plasmid, a lipid, a protein particle, a bacterial vector, a lysosome, a virus-like particle, a polymeric particle, an exosome, or a vault particle.

### Non-Viral Vectors

In some aspects, the nucleic acid or plasmid of interest can be administered using a non-viral vector. “Non-viral vector,” as used herein is meant to include naked DNA, chemical formulations containing naked DNA (e.g., a formulation of DNA and cationic compounds (e.g., dextran sulfate)), and naked DNA mixed with an adjuvant such as a viral particle (i.e., the DNA of interest is not contained within the viral particle, but the transforming formulation is composed of both naked DNA and viral particles (e.g., AAV particles) (see e.g., Curiel et al., Am. J. Respir. Cell Mol. Biol. 6:247-52 (1992)). Thus the “non-viral vector” can include vectors composed of DNA plus viral particles where the viral particles do not contain the DNA of interest within the viral genome.

In some aspects, the non-viral vector is a bacterial vector. See e.g., Baban et al., Bioeng Bugs., 1(6):385-394 (2010).

In some aspects, the nucleic acid or plasmid of interest can be complexed with polycationic substances such as poly-L-lysine or DEAC-dextran, targeting ligands, and/or DNA binding proteins (e.g., histones). DNA- or RNA-liposome complex formulations comprise a mixture of lipids which bind to genetic material (DNA or RNA) and facilitate delivery of the nucleic acid into the cell. Liposomes which can be used in accordance with the disclosure include DOPE (dioleyl phosphatidyl ethanol amine), CUDMEDA (N-(5-cholestrum-3-β-ol 3-urethanyl)-N,N′-dimethylethylene diamine).

Lipids which can be used in accordance with the disclosure include, but are not limited to, DOPE (Dioleoyl phosphatidylethanolamine), cholesterol, and CUDMEDA (N-(5-cholestrum-3-ol 3 urethanyl)-N,N′-dimethylethylenediamine). As an example, DNA can be administered in a solution containing one of the following cationic liposome formulations: Lipofectin™ (LTI/BRL), Transfast™ (Promega Corp), Tfx50™ (Promega Corp), Tfx10™ (Promega Corp), or Tfx20™ (Promega Corp). The concentration of the liposome solutions range from about 2.5% to 15% volume:volume, preferably about 6% to 12% volume:volume. Further exemplary methods and compositions for formulation of nucleic acid (e.g., DNA, including DNA or RNA not contained within a viral particle) for delivery according to the method of the disclosure are described in U.S. Pat. Nos. 5,892,071; 5,744,625; 5,925,623; 5,527,928; 5,824,812; 5,869,715.

In some aspects, protein particles can be used in accordance with the disclosure for polymer-based gene delivery. See e.g., Putnam et al., PNAS 98 (3): 1200-1205 (2001).

In some aspects, the nucleic acid or plasmid of interest can be administered as a chemical formulation of DNA or RNA coupled to a carrier molecule (e.g., an antibody or a receptor ligand) which facilitates delivery to host cells for the purpose of altering the biological properties of the host cells. The term “chemical formulations” refers to modifications of nucleic acids to allow coupling of the nucleic acid compounds to a carrier molecule such as a protein or lipid, or derivative thereof.

### Viral Vectors

In general, viral delivery vectors can be composed of a viral particle derived from a naturally-occurring virus which has been genetically altered to render the virus replication-defective and to express a recombinant gene of interest in accordance with the disclosure. Once the virus delivers its genetic material to a cell, it does not generate additional infectious virus but does introduce exogenous recombinant genes into the cell, preferably into the genome of the cell.

In some aspects, the viral vector is a retrovirus, an adenovirus, an adeno-associated virus (AAV), a herpes simplex virus (HSV), a cytomegalovirus (CMV), a vaccinia or a poliovirus vectors. In some aspects, retroviral vectors are less preferred since retroviruses require replicating cells and secretory glands are composed of mostly slowly replicating and/or terminally differentiated cells. In some aspects, adenovirus and AAV are preferred viral vectors since this virus efficiently infects slowly replicating and/or terminally differentiated cells. In some aspects, the delivery vector (e.g., viral vector) is selected from the group consisting of an adeno-associated viral (AAV) vector, an adenoviral vector, a lentiviral vector, or a retroviral vector.

Where a replication-deficient virus is used as the viral vector, the production of infective virus particles containing either DNA or RNA corresponding to the DNA of interest can be produced by introducing the viral construct into a recombinant cell line which provides the missing components essential for viral replication. In some aspects, transformation of the recombinant cell line with the recombinant viral vector will not result in production of replication-competent viruses, e.g., by homologous recombination of the viral sequences of the recombinant cell line into the introduced viral vector. Methods for production of replication-deficient viral particles containing a nucleic acid of interest are well known in the art and are described in, e.g., Rosenfeld et al., Science 252:431-434 (1991) and Rosenfeld et al., Cell 68:143-155 (1992) (adenovirus); U.S. Pat. No. 5,139,941 (adeno-associated virus); U.S. Pat. No. 4,861,719 (retrovirus); and U.S. Pat. No. 5,356,806 (vaccinia virus).

### Lentiviral Expression Constructs

Lentiviruses are complex retroviruses that in addition to the common retroviral genes gag, pol and env, contain other genes with regulatory or structural function. The higher complexity enables the lentivirus to modulate the life cycle thereof, as in the course of latent infection.

A typical lentivirus is the human immunodeficiency virus (HIV), the etiologic agent of AIDS. In vivo, HIV can infect terminally differentiated cells that rarely divide, such as lymphocytes and macrophages. In vitro, HIV can infect primary cultures of monocyte-derived macrophages (MDM) as well as HeLa-Cd4 or T lymphoid cells arrested in the cell cycle by treatment with aphidicolin or γ irradiation.

Infection of cells is dependent on the active nuclear import of HIV preintegration complexes through the nuclear pores of the target cells. That occurs by the interaction of multiple, partly redundant, molecular determinants in the complex with the nuclear import machinery of the target cell. Identified determinants include a functional nuclear localization signal (NLS) in the gag matrix (MA) protein, the karyophilic virion-associated protein, vpr, and a C-terminal phosphotyrosine residue in the gag MA protein.

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

In some aspects, the recombinant lentivirus is capable of infecting a non-dividing cell by transfecting a suitable host cell with two or more vectors carrying the packaging functions, namely gag, pol and env, as well as rev and tat. In some examples, vectors lacking a functional tat gene are desirable. Thus, for example, a first vector can provide a nucleic acid encoding a viral gag and a viral pol and another vector can provide a nucleic acid encoding a viral env to produce a packaging cell. Introducing a vector providing a heterologous gene, identified as a transfer vector, into that packaging cell yields a producer cell which releases infectious viral particles carrying the foreign gene of interest.

The gag, pol and env genes of the vectors of interest also are known in the art. Thus, the relevant genes are cloned into the selected vector and then used to transform the target cell of interest.

According to the above-indicated configuration of vectors and foreign genes, the second vector can provide a nucleic acid encoding a viral envelope (env) gene. The env gene can be derived from any virus, including retroviruses. The env preferably is an amphotropic envelope protein which allows transduction of cells of human and other species.

It may be desirable to target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific. Retroviral vectors can be made target-specific by inserting, for example, a glycolipid or a protein. Targeting often is accomplished by using an antigen-binding portion of an antibody or a recombinant antibody-type molecule, such as a single chain antibody, to target the retroviral vector. Those of skill in the art will know of, or can readily ascertain without undue experimentation, specific methods to achieve delivery of a retroviral vector to a specific target.

Examples of retroviral-derived env genes include, but are not limited to: Moloney murine leukemia virus (MoMuLV or MMLV), Harvey murine sarcoma virus (HaMuSV or HSV), murine mammary tumor virus (MuMTV or MMTV), gibbon ape leukemia virus (GaLV or GALV), human immunodeficiency virus (HIV) and Rous sarcoma virus (RSV). Other env genes such as Vesicular stomatitis virus (VSV) protein G (VSV G), that of hepatitis viruses and of influenza also can be used.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

In some aspects, the lentiviral genome as present in said lentiviral vector further comprises a promoter sequence operably linked to the nucleotide sequence encoding any of the fusion proteins disclosed herein.

### V. Adeno-Associated Virus (AAV) Vectors and Capsids

AAV, a parvovirus belonging to the genus Dependovirus, has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

In some aspects, AAV vectors of the present disclosure can comprise or be derived from any natural or recombinant AAV serotype. According to the present disclosure, the AAV serotype can be, but is not limited to, AAV1, AAV2, AAV3, AAV4, AAV5, AAV6, AAV7, AAV8, AAVrh8, AAV9, AAV10, AAVrh10, AAV11, and AAV12, and variants thereof.

In some aspects, the AAV is a self-complementary adeno-associated virus (scAAV).

In some aspects, provided herein is an adeno-associated virus (AAV) vector comprising a polynucleotide comprising a promoter operably linked to a nucleic acid encoding a CR2-CR1 fusion protein. In some aspects, the polynucleotide comprises a nucleic acid sequence having at least 85%, at least 90%, at least 95%, at least 96%, at least 97%, at least 98%, at least 99%, or 100% identity to any of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, or SEQ ID NO: 61. In some aspects, the nucleic acid encoding the polynucleotide comprises a nucleic acid sequence having at least about 85%, at least about 90%, at least about 95%, at least about 96%, at least about 97%, at least about 98%, at least about 99%, or 100% identity to any of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, or SEQ ID NO: 61.

In some aspects, the polynucleotide comprises a nucleic acid sequence having at least 85%, at least 90%, at least 95%, at least 96%, at least 97%, at least 98%, at least 99%, or 100% identity to SEQ ID NO: 29. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 29.

In some aspects, the polynucleotide comprises a nucleic acid sequence having at least 85%, at least 90%, at least 95%, at least 96%, at least 97%, at least 98%, at least 99%, or 100% identity to SEQ ID NO: 30. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 30.

In some aspects, the polynucleotide comprises a nucleic acid sequence having at least 85%, at least 90%, at least 95%, at least 96%, at least 97%, at least 98%, at least 99%, or 100% identity to SEQ ID NO: 31. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 31.

In some aspects, the polynucleotide comprises a nucleic acid sequence having at least 85%, at least 90%, at least 95%, at least 96%, at least 97%, at least 98%, at least 99%, or 100% identity to SEQ ID NO: 32. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 32.

In some aspects, the polynucleotide comprises a nucleic acid sequence having at least 85%, at least 90%, at least 95%, at least 96%, at least 97%, at least 98%, at least 99%, or 100% identity to SEQ ID NO: 33. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 33.

In some aspects, the polynucleotide comprises a nucleic acid sequence having at least 85%, at least 90%, at least 95%, at least 96%, at least 97%, at least 98%, at least 99%, or 100% identity to SEQ ID NO: 34. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 34.

In some aspects, the polynucleotide comprises a nucleic acid sequence having at least 85%, at least 90%, at least 95%, at least 96%, at least 97%, at least 98%, at least 99%, or 100% identity to SEQ ID NO: 35. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 35.

In some aspects, the polynucleotide comprises a nucleic acid sequence having at least 85%, at least 90%, at least 95%, at least 96%, at least 97%, at least 98%, at least 99%, or 100% identity to SEQ ID NO: 36. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 36.

In some aspects, the polynucleotide comprises a nucleic acid sequence having at least 85%, at least 90%, at least 95%, at least 96%, at least 97%, at least 98%, at least 99%, or 100% identity to SEQ ID NO: 37. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 37.

In some aspects, the polynucleotide comprises a nucleic acid sequence having at least 85%, at least 90%, at least 95%, at least 96%, at least 97%, at least 98%, at least 99%, or 100% identity to SEQ ID NO: 38. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 38.

In some aspects, the polynucleotide comprises a nucleic acid sequence having at least 85%, at least 90%, at least 95%, at least 96%, at least 97%, at least 98%, at least 99%, or 100% identity to SEQ ID NO: 39. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 39.

In some aspects, the polynucleotide comprises a nucleic acid sequence having at least 85%, at least 90%, at least 95%, at least 96%, at least 97%, at least 98%, at least 99%, or 100% identity to SEQ ID NO: 40. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 40.

In some aspects, the polynucleotide comprises a nucleic acid sequence having at least 85%, at least 90%, at least 95%, at least 96%, at least 97%, at least 98%, at least 99%, or 100% identity to SEQ ID NO: 41. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 41.

In some aspects, the polynucleotide comprises a nucleic acid sequence having at least 85%, at least 90%, at least 95%, at least 96%, at least 97%, at least 98%, at least 99%, or 100% identity to SEQ ID NO: 42. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 42.

In some aspects, the polynucleotide comprises a nucleic acid sequence having at least 85%, at least 90%, at least 95%, at least 96%, at least 97%, at least 98%, at least 99%, or 100% identity to SEQ ID NO: 43. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 43.

In some aspects, the polynucleotide comprises a nucleic acid sequence having at least 85%, at least 90%, at least 95%, at least 96%, at least 97%, at least 98%, at least 99%, or 100% identity to SEQ ID NO: 44. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 44.

In some aspects, the polynucleotide comprises a nucleic acid sequence having at least 85%, at least 90%, at least 95%, at least 96%, at least 97%, at least 98%, at least 99%, or 100% identity to SEQ ID NO: 45. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 45.

In some aspects, the polynucleotide comprises a nucleic acid sequence having at least 85%, at least 90%, at least 95%, at least 96%, at least 97%, at least 98%, at least 99%, or 100% identity to SEQ ID NO: 46. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 46.

In some aspects, the polynucleotide comprises a nucleic acid sequence having at least 85%, at least 90%, at least 95%, at least 96%, at least 97%, at least 98%, at least 99%, or 100% identity to SEQ ID NO: 47. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 47.

In some aspects, the polynucleotide comprises a nucleic acid sequence having at least 85%, at least 90%, at least 95%, at least 96%, at least 97%, at least 98%, at least 99%, or 100% identity to SEQ ID NO: 48. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 48.

In some aspects, the polynucleotide comprises a nucleic acid sequence having at least 85%, at least 90%, at least 95%, at least 96%, at least 97%, at least 98%, at least 99%, or 100% identity to SEQ ID NO: 49. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 49.

In some aspects, the polynucleotide comprises a nucleic acid sequence having at least 85%, at least 90%, at least 95%, at least 96%, at least 97%, at least 98%, at least 99%, or 100% identity to SEQ ID NO: 50. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 50.

In some aspects, the polynucleotide comprises a nucleic acid sequence having at least 85%, at least 90%, at least 95%, at least 96%, at least 97%, at least 98%, at least 99%, or 100% identity to SEQ ID NO: 51. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 51.

In some aspects, the polynucleotide comprises a nucleic acid sequence having at least 85%, at least 90%, at least 95%, at least 96%, at least 97%, at least 98%, at least 99%, or 100% identity to SEQ ID NO: 52. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 52.

In some aspects, the polynucleotide comprises a nucleic acid sequence having at least 85%, at least 90%, at least 95%, at least 96%, at least 97%, at least 98%, at least 99%, or 100% identity to SEQ ID NO: 53. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 53.

In some aspects, the polynucleotide comprises a nucleic acid sequence having at least 85%, at least 90%, at least 95%, at least 96%, at least 97%, at least 98%, at least 99%, or 100% identity to SEQ ID NO: 54. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 54.

In some aspects, the polynucleotide comprises a nucleic acid sequence having at least 85%, at least 90%, at least 95%, at least 96%, at least 97%, at least 98%, at least 99%, or 100% identity to SEQ ID NO: 55. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 55.

In some aspects, the polynucleotide comprises a nucleic acid sequence having at least 85%, at least 90%, at least 95%, at least 96%, at least 97%, at least 98%, at least 99%, or 100% identity to SEQ ID NO: 56. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 56.

In some aspects, the polynucleotide comprises a nucleic acid sequence having at least 85%, at least 90%, at least 95%, at least 96%, at least 97%, at least 98%, at least 99%, or 100% identity to SEQ ID NO: 57. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 57.

In some aspects, the polynucleotide comprises a nucleic acid sequence having at least 85%, at least 90%, at least 95%, at least 96%, at least 97%, at least 98%, at least 99%, or 100% identity to SEQ ID NO: 58. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 58.

In some aspects, the polynucleotide comprises a nucleic acid sequence having at least 85%, at least 90%, at least 95%, at least 96%, at least 97%, at least 98%, at least 99%, or 100% identity to SEQ ID NO: 59. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 59.

In some aspects, the polynucleotide comprises a nucleic acid sequence having at least 85%, at least 90%, at least 95%, at least 96%, at least 97%, at least 98%, at least 99%, or 100% identity to SEQ ID NO: 60. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 60.

In some aspects, the polynucleotide comprises a nucleic acid sequence having at least 85%, at least 90%, at least 95%, at least 96%, at least 97%, at least 98%, at least 99%, or 100% identity to SEQ ID NO: 61. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 61.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to any of SEQ ID NOs: 77-109.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 77. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 77.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 78. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 78.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 79. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 79.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 80. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 80.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 81. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 81.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 82. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 82.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 83. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 83.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 84. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 84.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 85. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 85.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 86. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 86.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 87. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 87.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 88. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 88.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 89. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 89.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 90. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 90.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 91. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 91.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 92. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 92.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 93. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 93.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 94. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 94.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 95. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 95.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 96. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 96.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 97. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 97.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 98. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 98.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 99. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 99.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 100. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 100.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 101. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 101.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 102. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 102.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 103. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 103.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 104. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 104.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 105. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 105.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 106. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 106.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 107. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 107.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 108. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 108.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 109. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 109.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to any of SEQ ID NOs: 110-113.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 110. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 110.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 111. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 111.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 112. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 112.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 113. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 113.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to any of SEQ ID NOs: 114-135.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 114. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 114.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 115. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 115.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 116. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 116.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 117. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 117.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 118. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 118.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 119. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 119.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 120. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 120.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 121. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 121.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 122. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 122.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 123. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 123.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 124. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 124.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 125. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 125.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 126. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 126.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 127. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 127.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 128. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 128.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 129. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 129.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 130. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 130.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 131. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 131.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 132. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 132.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 133. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 133.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 134. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 134.

In some aspects, the nucleic acid encoding a CR2-CR1 fusion protein comprises a nucleic acid sequence having at least 85%, at least 90%, at least 95%, at least 96%, at least 97%, at least 98%, at least 99%, or 100% identity to SEQ ID NO: 135. In some aspects, the nucleic acid encoding a CR2-CR1 fusion protein comprises a nucleic acid sequence having at least about 85%, at least about 90%, at least about 95%, at least about 96%, at least about 97%, at least about 98%, at least about 99%, or 100% identity to SEQ ID NO: 135.

In some aspects, the polynucleotide comprises a nucleic acid sequence having at least 85%, at least 90%, at least 95%, at least 96%, at least 97%, at least 98%, at least 99%, or 100% identity to any of SEQ ID NO: 141 or 142.

In some aspects, the polynucleotide comprises a nucleic acid sequence having at least 85%, at least 90%, at least 95%, at least 96%, at least 97%, at least 98%, at least 99%, or 100% identity to SEQ ID NO: 141. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 141.

In some aspects, the polynucleotide comprises a nucleic acid sequence having at least 85%, at least 90%, at least 95%, at least 96%, at least 97%, at least 98%, at least 99%, or 100% identity to SEQ ID NO: 142. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 142.

In some aspects, the AAV vector, the plasmid, or the nucleic acid disclosed herein comprises a promoter.

In some aspects, the promoter comprises a CBA promoter, a CMV promoter, an EF-1a (Elongation Factor 1a) promoter, a RSV (Rous Sarcoma Virus) promoter, an Ubiquitin (UbC) promoter, a CAG promoter, a smCBA promoter or any combination thereof.

In some aspects, the promoter is a smCBA promoter.

In some aspects, the promoter is a constitutively active promoter, a cell-type specific promoter, or an inducible promoter. In some aspects, the promoter is a CMV promoter. In some aspects, the promoter is a CAG promoter.

Promoters can be naturally occurring or non-naturally occurring. Non-limiting examples of promoters include viral promoters and mammalian promoters. In some aspects, the promoters can be human promoters. In some aspects, the promoter can be truncated. In some aspects, the promoter comprises a human elongation factor 1a-subunit (EF1a), cytomegalovirus (CMV) immediate-early enhancer and/or promoter (SEQ ID NO: 68), chicken β-actin (CBA) and its derivative CAG, β glucuronidase (GUSB), or ubiquitin C (UBC). In some aspects, the promoter is a CBA promoter, a CMV promoter, an EF-1a (Elongation Factor 1a) promoter, aRSV (Rous Sarcoma Virus) promoter, an Ubiquitin (UbC) promoter, or any combination thereof. In some aspects, tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

In some aspects, the promoter can be less than 1 kb. In some aspects, the promoter can have a length between about 15-20, about 10-50, about 20-30, about 30-40, about 40-50, about 50-60, about 50-100, about 60-70, about 70-80, about 80-90, about 90-100, about 100-110, about 100-150, about 110-120, about 120-130, about 130-140, about 140-150, about 150-160, about 150-200, about 160-170, about 170-180, about 180-190, about 190-200, about 200-210, about 200-250, about 210-220, about 220-230, about 230-240, about 240-250, about 250-260, about 250-300, about 260-270, about 270-280, about 280-290, about 290-300, about 200-300, about 200-400, about 200-500, about 200-600, about 200-700, about 200-800, about 300-400, about 300-500, about 300-600, about 300-700, about 300-800, about 400-500, about 400-600, about 400-700, about 400-800, about 500-600, about 500-700, about 500-800, about 600-700, about 600-800 or about 700-800 nucleotides.

In some aspects, the promoter is a ubiquitous promoter. Non-limiting examples of ubiquitous promoters include, e.g., CMV, CBA (including derivatives CAG, CBh, etc.), EF-1a, PGK, UBC, GUSB (hGBp), and UCOE (promoter of HNRPA2B1-CBX3).

In some aspects, the promoter is not cell specific. In some aspects, the promoter is an ubiquitin c (UBC) promoter. The UBC promoter can have a size of 300-350 nucleotides. In some aspects, the UBC promoter is 332 nucleotides. In some aspects, the promoter is a 0-glucuronidase (GUSB) promoter. The GUSB promoter can have a size of 350-400 nucleotides. In some aspects, the GUSB promoter is 378 nucleotides. In some aspects, the promoter is a neurofilament light (NFL) promoter. The NFL promoter can have a size of 600-700 nucleotides. In some aspects, the NFL promoter is 650 nucleotides. In some aspects, the construct can be AAV-promoter-CMV/globin intron-modulatory polynucleotide-RBG, where the AAV can be self-complementary and the AAV can be the DJ serotype.

In some aspects, the AAV vector, the plasmid, or the nucleic acid disclosed herein further comprises a Kozak sequence. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another G.

In some aspects, the AAV vector, the plasmid, or the nucleic acid disclosed herein further comprises an enhancer. In some aspects, the enhancer is a CMV enhancer (SEQ ID NO: 69).

In some aspects, the AAV vector, the plasmid, or the nucleic acid disclosed herein further comprises an Internal Ribosome Entry Site (IRES) (SEQ ID NO: 70).

In some aspects, the AAV vector, the plasmid, or the nucleic acid disclosed herein comprises further comprises a poly(A) sequence. In some aspects, the polyadenylation sequence or “polyA sequence” can range from 50 to about 500 nucleotides in length.

In some aspects, the polyadenylation sequence is about 50-100, about 50-150, about 50-160, about 50-200, about 60-100, about 60-150, about 60-160, about 60-200, about 70-100, about 70-150, about 70-160, about 70-200, about 80-100, about 80-150, about 80-160, about 80-200, about 90-100, about 90-150, about 90-160, or about 90-200 nucleotides in length.

In some aspects, the polyadenylation sequence is a rabbit globin polyadenylation (poly A) signal sequence. In some aspects, the polyadenylation sequence is a human growth hormone polyadenylation (poly A) signal sequence. In some aspects, the polyadenylation sequence is a bovine growth hormone polyadenylation (poly A) signal sequence. In some aspects, the polyadenylation sequence is a synthetic polyadenylation (poly A) signal sequence. In some aspects, the polyadenylation sequence is a SV40 polyA signal sequence.

In some aspects, the poly(A) sequence comprises a human growth hormone poly(A) sequence, a bovine growth hormone poly(A) sequence, or a synthetic poly(A) sequence.

In some aspects, the poly(A) sequence is a synthetic poly(A) sequence.

In some aspects, the synthetic poly(A) sequence comprises a nucleic acid sequence corresponding to SEQ ID NO: 71.

In some aspects, the poly(A) sequence is a bovine growth hormone poly(A) sequence.

In some aspects, the bovine growth hormone poly(A) sequence comprises a nucleic acid sequence corresponding to SEQ ID NO: 72.

In some aspects, the poly(A) sequence is a human growth hormone poly(A) sequence.

In some aspects, the human growth hormone poly(A) sequence comprises a nucleic acid sequence corresponding to SEQ ID NO: 73.

In some aspects, the polynucleotide further comprises two inverted terminal repeat (ITR) sequences.

In some aspects, the pair of ITR sequences are derived from an AAV serotype selected from AAV1, AAV2, AAV3a, AAV3b, AAV4, AAV5, AAV6, AAV7, AAV8, AAV9, AAV10, AAV11, AAV12, or variants thereof.

In some aspects, the AAV vector comprises at least one ITR region and a payload region, e.g., a polynucleotide or expression cassette for expression of the fusion protein. In some aspects, the AAV vector comprises two ITRs. In some aspects, these two ITRs flank the payload region at the 5′ and 3′ ends. The ITRs can function as origins of replication comprising recognition sites for replication. In some aspects, the ITRs comprise sequence regions that can be complementary and symmetrically arranged. In some aspects, the ITRs incorporated into the AAV vector of the present disclosure can be comprised of naturally occurring polynucleotide sequences or recombinantly derived polynucleotide sequences.

The ITRs can be derived from the same serotype as the desired capsid, selected from any of the serotypes disclosed herein, or a derivative thereof. The ITR can be of a different serotype from the desired capsid. In some aspects, the ITRs are of the same serotype as one another. In some aspects, the ITRs are of different serotypes. Non-limiting examples include zero, one or both of the ITRs having the same serotype as the desired capsid.

Independently, each ITR can be about 75 to about 175 nucleotides in length. In some aspects, the ITR can be about 100-150 nucleotides in length, about 140-150 nucleotides in length or about 140-155 nucleotides in length. Non-limiting examples of ITR length are about 140, about 141, about 142, about 145 nucleotides in length, and those having at least 95% identity thereto.

In some aspects, the AAV vector comprises at least one inverted terminal repeat having a length of about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, or about 170-175 nucleotides.

In some aspects, the length of a first and/or a second ITR region for AAV vector can be about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, or about 170-175 nucleotides.

In some aspects, the nucleic acid of interest is located near the 5′ end of the flip ITR in the vector. In some aspects, the nucleic acid of interest is located near the 3′ end of the flip ITR in the vector. In some aspects, the nucleic acid of interest is located near the 5′ end of the flop ITR in the vector. In some aspects, the nucleic acid of interest is located near the 3′ end of the flop ITR in the vector. In some aspects, the nucleic acid of interest is located between the 5′ end of the flip ITR and the 3′ end of the flop ITR in the vector. In some aspects, the nucleic acid of interest is located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in the vector.

In some aspects, the nucleic acid of interest is located within about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or more than about 30 nucleotides downstream or upstream from the 5′ or 3′ end of an ITR (e.g., Flip or Flop ITR) in the vector.

As another non-limiting example, the nucleic acid of interest is located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream or upstream from the 5′ or 3′ end of an ITR (e.g., Flip or Flop ITR) in the vector.

In some aspects, the nucleic acid of interest is located within the first about 1%, about 2%, about 3%, about 4%, about 5%, about 6%, about 7%, about 8%, about 9%, about 10%, about 15%, about 20%, about 25% or more than about 25% of the nucleotides upstream from the 5′ or 3′ end of an ITR (e.g., Flip or Flop ITR) in the vector.

As another non-limiting example, the nucleic acid of interest is located with the first about 1-5%, about 1-10%, about 1-15%, about 1-20%, about 1-25%, about 5-10%, about 5-15%, about 5-20%, about 5-25%, about 10-15%, about 10-20%, about 10-25%, about 15-20%, about 15-25%, or about 20-25% downstream from the 5′ or 3′ end of an ITR (e.g., Flip or Flop ITR) in the vector.

In some aspects, the polynucleotide further comprises an enhancer sequence.

In some aspects, the enhancer is a CMV enhancer sequence.

In some aspects, the polynucleotide further comprises an intron sequence.

In some aspects, the intron sequence is a SV40 intron sequence or a CAG intron sequence.

In some aspects, the AAV vector comprises an AAV capsid of a serotype selected from the group consisting of AAV1, AAV2, AAV3, AAV4, AAV5, AAV6, AAV7, AAV8, AAVrh8, AAV9, AAV10, AAVrh10, AAV11, and AAV12.

In some aspects, the AAV vector comprises an AAV2 serotype capsid.

In some aspects, provided herein is a polynucleotide comprising a nucleic acid sequence encoding any of the fusion proteins disclosed herein.

In some aspects, provided herein is a polynucleotide comprising comprises a nucleic acid sequence having at least 85%, at least 90%, at least 95%, at least 96%, at least 97%, at least 98%, at least 99%, or 100% identity to any of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, or SEQ ID NO: 61.

In some aspects, provided herein is a polynucleotide comprising comprises a nucleic acid sequence having at least about 85%, at least about 90%, at least about 95%, at least about 96%, at least about 97%, at least about 98%, at least about 99%, or 100% identity to any of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, or SEQ ID NO: 61.

In some aspects, provided herein is a polynucleotide comprising comprises a nucleic acid sequence having at least 85%, at least 90%, at least 95%, at least 96%, at least 97%, at least 98%, at least 99%, or 100% identity to SEQ ID NO: 30. In some aspects, provided herein is a polynucleotide comprising comprises a nucleic acid sequence having at least about 85%, at least about 90%, at least about 95%, at least about 96%, at least about 97%, at least about 98%, at least about 99%, or 100% identity to SEQ ID NO: 30.

In some aspects, the nucleic acid sequence has been modified to reduce the number of CpG sites.

In some aspects, provided herein is an adeno-associated (AAV) capsid comprising any of the polynucleotides disclosed herein.

In some aspects, the AAV capsid serotype is selected from the group consisting of AAV1, AAV2, AAV3, AAV4, AAV5, AAV6, AAV7, AAV8, AAVrh8, AAV9, AAV10, AAVrh10, AAV11, and AAV12. In some aspects, the AAV capsid serotype is selected from or is derived from AAV2, AAV5, or AAV8. In some aspects, the AAV capsid serotype is selected AAV2.

In some aspects, the AAV capsid is modified relative to the wild-type serotype. In some aspects, the modified AAV capsid serotype is an AAV2 Y→F mutant including AAV2 QuadYF (e.g., Y272F, Y444F, Y500F and Y730F), AAV2 QuadYF+TV (also referred to as “AAV2-QUAD+”, e.g., including Y272F, Y444F, Y500F, Y730F, and T491V), AAV2.7m8 (see, e.g., U.S. Pat. No. 9,587,282B2); AAV2.5T.LSV1 or AAV5.LSV1 (WO2020219933A1), rAAV2/2[MAX](see, e.g., Reid, C. A., Ertel, K. J. & Lipinski, D. M. Improvement of Photoreceptor Targeting via Intravitreal Delivery in Mouse and Human Retina Using Combinatory rAAV2 Capsid Mutant Vectors. Invest Ophth Vis Sci 58, 6429-6439 (2017). doi.org/10.1167/iovs.17-22281).

In some aspects, the disclosure is directed to a viral particle comprising the vector genome of any of the AAV vectors described herein. In some aspects, the viral particle is an AAV particle. In some aspects, the AAV particle is produced by any of the methods of packaging described herein or known in the art.

In some aspects, the disclosure is directed to a plurality of viral particles or AAV particles. In some aspects, the viral particle or AAV particles are isolated or purified.

The present disclosure also provides for cells containing the AAV vector disclosed herein. In some aspect, the cell comprises mammalian cells. In some aspects, the cell provides helper functions that package the AAV vector into a viral particle. In some aspects, the cell provides AAV helper functions. In some aspects, the cell provides AAV Rep and/or Cap proteins. In some aspects, the cell is stably or transiently transfected with polynucleotide(s) encoding Rep and/or Cap protein sequence(s). In some aspects, the cell provides Rep78 or/and Rep68 proteins. In some aspects, the cell is stably or transiently transfected with Rep78 and Rep68 proteins polynucleotide encoding sequence(s).

In some aspects, the present disclosure provides for a method for packaging a nucleic acid of interest in an AAV capsid. In some aspects, the method comprises transfecting a cell in vitro with any AAV vector described herein and one or more plasmids comprising Rep/Cap genes and adenovirus genes. In some aspects, the nucleic acid of interest is packaged into the AAV capsid.

In some aspects, an AAV particle is produced by a method comprising:
(1) co-transfecting competent bacterial cells with a bacmid vector and either a viral construct vector and/or AAV payload construct vector, (2) isolating the resultant viral construct expression vector and AAV payload construct expression vector and separately transfecting viral replication cells, (3) isolating and purifying resultant payload and viral construct particles comprising viral construct expression vector or AAV payload construct expression vector, (4) co-infecting a viral replication cell with both the AAV payload and viral construct particles comprising viral construct expression vector or AAV payload construct expression vector, and (5) harvesting and purifying the viral particle comprising a parvoviral genome.

In some aspects, the present disclosure provides a method for producing an AAV particle comprising the steps of (1) simultaneously co-transfecting mammalian cells, such as, but not limited to HEK293 cells, with: (i) AAV vector of the disclosure comprising a payload region (e.g., polynucleotide encoding a fusion protein), (ii) a construct expressing rep and cap genes and (iii) a helper construct, and (2) harvesting and purifying the AAV particle comprising a viral genome.

In some aspects, the AAV particles can be produced in a viral replication cell that comprises an insect cell. Growing conditions for insect cells in culture, and production of heterologous products in insect cells in culture are well known in the art, see, e.g., U.S. Pat. No. 6,204,059.

The viral replication cell can be selected from any biological organism, including prokaryotic (e.g., bacterial) cells, and eukaryotic cells, including, insect cells, yeast cells and mammalian cells. Viral replication cells can comprise mammalian cells such as A549, WEH1, 3T3, 10T1/2, BHK, MDCK, COS 1, COS 7, BSC 1, BSC 40, BMT 10, VERO, W138, HeLa, HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

Viral production disclosed herein describes processes and methods for producing AAV particles that contact a target cell to deliver a payload, e.g. a recombinant viral construct, which comprises a polynucleotide or expression construct disclosed herein.

In some aspects, the AAV particles can be produced in a viral replication cell that comprises a mammalian cell. Viral replication cells commonly used for production of recombinant AAV particles include, but are not limited to 293 cells, COS cells, HeLa cells, and KB cells.

In some aspects, AAV particles are produced in mammalian cells wherein all three VP proteins are expressed at a stoichiometry approaching 1:1:10 (VP1:VP2:VP3). In some aspects, the VP proteins are expressed at a stoichiometry such that VP3 is higher than VP1 and VP2. In some aspects, the VP proteins are expressed at a stoichiometry such that VP1 and VP2 are at approximately the same level. The regulatory mechanisms that allow this controlled level of expression include the production of two mRNAs, one for VP1, and the other for VP2 and VP3, produced by differential splicing.

In some aspects, AAV particles are produced in mammalian cells using a triple transfection method wherein a payload construct (e.g., an AAV vector disclosed herein), parvoviral Rep and parvoviral Cap and a helper construct are comprised within three different constructs. The triple transfection method of the three components of AAV particle production can be utilized to produce small lots of virus for assays including transduction efficiency, target tissue (tropism) evaluation, and stability.

In some aspects, the viral construct vector and the AAV vector can be each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses can be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see, e.g., Urabe, M. et al., J Virol. 2006 February; 80 (4): 1874-85, the contents of which are herein incorporated by reference in their entirety.

Production of AAV particles with baculovirus in an insect cell system can address known baculovirus genetic and physical instability. Baculovirus-infected viral producing cells are harvested into aliquots that can be cryopreserved in liquid nitrogen; the aliquots retain viability and infectivity for infection of large-scale viral producing cell culture (Wasilko D J et al., Protein Expr Purif 2009 June; 65(2): 122-32).

In some aspects, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some aspects, AAV particle production can be modified to increase the scale of production. Transfection of replication cells in large-scale culture formats can be carried out according to any methods known in the art.

In some aspects, cell culture bioreactors can be used for large scale viral production. In some cases, bioreactors comprise stirred tank reactors.

Cells of the disclosure, including, but not limited to viral production cells, can be subjected to cell lysis according to any methods known in the art. In some aspects, the cells of the disclosure are not subjected to cell lysis. Cell lysis can be carried out to obtain one or more agents (e.g. viral particles) present within any cells of the disclosure.

Cell lysis methods can be chemical or mechanical. Chemical cell lysis typically comprises contacting one or more cells with one or more lysis agent. Mechanical lysis typically comprises subjecting one or more cells to one or more lysis condition and/or one or more lysis force. In some aspects, chemical lysis can be used to lyse cells. As used herein, the term “lysis agent” refers to any agent that can aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions can include one or more buffering agents, solubilizing agents, surfactants, preservatives, cryoprotectants, enzymes, enzyme inhibitors and/or chelators.

Concentrations of salts can be increased or decreased to obtain an effective concentration for rupture of cell membranes. Lysis agents comprising detergents can include ionic detergents or non-ionic detergents. Detergents can function to break apart or dissolve cell structures including, but not limited to cell membranes, cell walls, lipids, carbohydrates, lipoproteins and glycoproteins.

In some aspects, mechanical cell lysis is carried out. Mechanical cell lysis methods can include the use of one or more lysis condition and/or one or more lysis force. As used herein, the term “lysis condition” refers to a state or circumstance that promotes cellular disruption. Lysis conditions can comprise certain temperatures, pressures, osmotic purity, salinity and the like. In some aspects, lysis conditions comprise increased or decreased temperatures. In some aspects, lysis conditions comprise changes in temperature to promote cellular disruption. Cell lysis carried out according to such aspects can include freeze-thaw lysis.

As used herein, the term “lysis force” refers to a physical activity used to disrupt a cell. Lysis forces can include, but are not limited to mechanical forces, sonic forces, gravitational forces, optical forces, electrical forces and the like. Cell lysis carried out by mechanical force is referred to herein as “mechanical lysis.” Mechanical forces that can be used according to mechanical lysis can include high shear fluid forces.

In some aspects, a method for harvesting AAV particles without lysis can be used for efficient and scalable AAV particle production. In a non-limiting example, AAV particles can be produced by culturing an AAV particle lacking a heparin binding site, thereby allowing the AAV particle to pass into the supernatant, in a cell culture, collecting supernatant from the culture; and isolating the AAV particle from the supernatant, as described in U.S. Patent Application 2009/0275107.

Cell lysates comprising viral particles can be subjected to clarification. Clarification refers to initial steps taken in purification of viral particles from cell lysates. Clarification serves to prepare lysates for further purification by removing larger, insoluble debris. Clarification steps can include, but are not limited to centrifugation and filtration.

In some aspects, AAV particles can be purified from clarified cell lysates by one or more methods of chromatography. Chromatography refers to any number of methods known in the art for separating out one or more elements from a mixture. Such methods can include, but are not limited to ion exchange chromatography (e.g. cation exchange chromatography and anion exchange chromatography), immunoaffinity chromatography and size-exclusion chromatography.

### VI. Pharmaceutical Compositions

In some aspects, provided herein is a pharmaceutical composition comprising any of the fusion proteins disclosed herein, any of the AAV vectors disclosed herein, any of the polynucleotides disclosed herein, or any of the AAV capsids disclosed herein.

In some aspects, the pharmaceutical composition is in a form suitable for administration to a subject in need thereof.

In some aspects, the pharmaceutical composition further comprises a pharmaceutically acceptable excipient or carrier.

In some aspects, pharmaceutically acceptable excipients or carriers are determined in part by the particular composition being administered, as well as by the particular method used to administer the composition.

Accordingly, there is a wide variety of suitable formulations of pharmaceutical compositions comprising a delivery vector of the present disclosure (e.g., an AAV vector) or a plurality thereof (see, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In some aspects, the pharmaceutical composition comprises more than one AAV vector, wherein each vector comprises at least one polynucleotide encoding a fusion protein disclosed herein.

Also provided herein are pharmaceutical compositions comprising delivery vectors disclosed herein (e.g., AAV vectors or AAV capsids) having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of vectors, e.g., AAV vectors described herein. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

Acceptable carriers, excipients, or stabilizers are nontoxic to recipients (e.g., animals or humans) at the dosages and concentrations employed.

Examples of carriers or diluents include, but are not limited to, water, saline, Ringer's solutions, dextrose solution, and 5% human serum albumin. Except insofar as any conventional media or compound is incompatible with the delivery vectors disclosed herein (e.g., AAV vectors or AAV capsids), use thereof in the compositions is contemplated. In some aspects, a pharmaceutical composition is formulated to be compatible with its intended route of administration.

The delivery vectors disclosed herein (e.g., AAV vectors or AAV capsids) can optionally be administered in combination with other therapeutic agents that are at least partly effective in treating the disease, disorder or condition for which the delivery vectors disclosed herein (e.g., AAV vectors or AAV capsids) are intended.

The delivery vectors disclosed herein (e.g., AAV vectors or AAV capsids) can be administered in any suitable form, either as a liquid solution or suspension, as a solid form suitable for liquid solution or suspension in a liquid solution.

### VII. Kits

The present disclosure also provides kits, or products of manufacture, comprising (i) any of the fusion proteins, the AAV vectors, the AAV capsids, or the pharmaceutical compositions disclosed herein, and (ii) optionally instructions for use (e.g., a package insert with instructions to perform any of the methods described herein).

In some aspects, the kit or product of manufacture comprises (i) an AAV vector comprising the fusion protein of the present disclosure (ii) optionally, an additional agent (e.g., rep/cap plasmid and/or helper plasmid), and (iii) optionally, instructions for use (e.g., a package insert with instructions to perform any of the methods described herein are also contemplated).

In some aspects, the kit or product of manufacture comprises (i) a fusion protein of the present disclosure, (ii) optionally, an additional therapeutic agent, and (iii) optionally, instructions for use (e.g., a package insert with instructions to perform any of the methods described herein are also contemplated).

One skilled in the art will readily recognize that the fusion proteins, the AAV vectors, the AAV capsids, or the pharmaceutical compositions of the present disclosure, can be readily incorporated into one of the established kit formats which are well known in the art.

### VIII. Administration

The fusion proteins, delivery vectors, or pharmaceutical compositions disclosed herein can be administered by any route which results in a therapeutically effective outcome. In some aspects, the methods disclosed herein can comprise administering a fusion protein, a delivery vector, a plasmid, or a nucleic acid of the present disclosure to the subject.

The fusion proteins, delivery vectors, or pharmaceutical compositions disclosed herein can be administered in any suitable form, either as a liquid solution or suspension, as a solid form suitable for liquid solution or suspension in a liquid solution.

In some aspects, the fusion proteins, delivery vectors, or pharmaceutical compositions disclosed herein is administered to a human.

A fusion protein, a delivery vector, a plasmid, a nucleic acid, or a pharmaceutical composition as disclosed herein for use in preventing, reducing or ameliorating the symptoms of, delaying, reverting, curing and/or treating a complement mediated disorder can be suitable for administration to a cell, tissue and/or an organ in vivo of individuals affected by or at risk of developing a complement mediated disorder, and may be administered in vivo, ex vivo or in vitro. Said fusion protein, delivery vector, plasmid, nucleic acid, or pharmaceutical composition can be directly or indirectly administrated to a cell, tissue and/or an organ in vivo of an individual affected by or at risk of developing a complement mediated disorder, and may be administered directly or indirectly in vivo, ex vivo or in vitro.

The fusion protein, delivery vector, plasmid, nucleic acid, or pharmaceutical composition disclosed herein can be administered to an individual via any effective route.

In some aspects, the fusion protein, delivery vector, plasmid, nucleic acid, or pharmaceutical composition disclosed herein is administered to the kidney, liver, lung, heart, or central nervous system.

In some aspects, the fusion protein, delivery vector, plasmid, nucleic acid, or pharmaceutical composition disclosed herein is administered by intravenous, intrathecal, intrahepatical, parenteral, topical, oral, subcutaneous, intra-arterial, intradermal, transdermal, rectal, intracranial, intraperitoneal, intranasal, intratumoral, intramuscular, intraarticular, intraventricular routes or as inhalants.

In some aspects, the fusion protein, delivery vector, plasmid, nucleic acid, or pharmaceutical composition disclosed herein is administered directly to the eye or the eye tissue. In some aspects, the fusion protein, delivery vector, plasmid, nucleic acid, or pharmaceutical composition disclosed herein are administered by injection to the eye (intraocular injection) or to the tissues associated with the eye. The fusion protein, delivery vector, plasmid, nucleic acid, or pharmaceutical composition disclosed herein can be administered, for example, by intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, posterior juxtascleral delivery, or suprachoroidal injection. These methods are known in the art. For example, for a description of exemplary periocular routes for retinal drug delivery, see Periocular routes for retinal drug delivery, Raghava et al. (2004), Expert Opin. Drug Deliv. 1(1):99-114. The fusion protein, delivery vector, plasmid, nucleic acid, or pharmaceutical composition disclosed herein may be administered, for example, to the vitreous, aqueous humor, sclera, conjunctiva, the area between the sclera and conjunctiva, the retina choroids tissues, macula, or other area in or proximate to the eye of an individual. The fusion protein, delivery vector, plasmid, nucleic acid, or pharmaceutical composition disclosed herein can also be administered to the individual as an implant. Preferred implants are biocompatible and/or biodegradable sustained release formulations which gradually release the compounds over a period of time. Ocular implants for drug delivery are well-known in the art. See, e.g., U.S. Pat. Nos. 5,501,856, 5,476,511, and 6,331,313. The fusion protein, delivery vector, plasmid, nucleic acid, or pharmaceutical composition disclosed herein can also be administered to the individual using iontophoresis, including, but are not limited to, the ionophoretic methods described in U.S. Pat. No. 4,454,151 and U.S. Pat. App. Pub. Nos. 2003/0181531 and 2004/0058313.

In some aspects, the fusion protein, delivery vector, plasmid, nucleic acid, or pharmaceutical composition disclosed herein is administered by suprachoroidal injection. In some aspects, the suprachoroidal injection comprises a device for delivering the fusion protein, delivery vector, plasmid, nucleic acid, or pharmaceutical composition disclosed herein to the suprachoroidal space. In some aspects, the device is an injector device. In some aspects, the devise is inserted into the sclera. In some aspects, the device is used to create a path into the Suprachoroidal space (e.g., by extending a separator between the choroid and sclera and then retracting the separator, which creates a channel to the suprachoroidal space). In some aspects, the fusion protein, delivery vector, plasmid, nucleic acid, or pharmaceutical composition disclosed herein is injected into the channel created by the device.

Certain aspects of the disclosure are directed to suprachoroidal administration of a fusion protein, a delivery vector comprising a nucleic acid encoding the fusion protein (e.g., an AAV vector), or pharmaceutical composition comprising the fusion protein or the delivery vector to a subject in need thereof, wherein the fusion protein comprises a complement receptor 1 (CR1) protein or a functional fragment thereof (e.g., a truncated CR1 protein) disclosed herein.

In some aspects, the fusion protein comprises a CR1 protein or functional fragment thereof comprising Short Consensus Repeats (SCRs) 1-4, 7-11, and 14-17. In some aspects, the fusion protein comprises a CR1 protein or functional fragment thereof that consists essentially of SCRs 1-4, 7-11, and 14-17. In some aspects, the fusion protein comprises a CR1 protein or functional fragment thereof that does not contain SCRs 5-6, 12-13, and 18-30. In some aspects, the fusion protein comprises a CR1 protein or functional fragment thereof comprising SCRs 1-4, 7-11, and 14-17, and does not contain SCRs 5-6, 12-13, and 18-30.

In some aspects, the fusion protein comprises a CR1 protein or functional fragment thereof comprising SCRs 1-14. In some aspects, the fusion protein comprises a CR1 protein or functional fragment thereof that consists essentially of SCRs 1-14. In some aspects, the fusion protein comprises a CR1 protein or functional fragment thereof does not contain SCRs 15-30. In some aspects, the fusion protein comprises a CR1 protein or functional fragment thereof comprising SCRs 1-14, and does not contain SCRs 15-30.

In some aspects, the fusion protein comprises a CR1 protein or functional fragment thereof that comprises SCRs 1-3, 7-10, and 14-17. In some aspects, the fusion protein comprises a CR1 protein or functional fragment thereof that consists essentially of SCRs 1-3, 7-10, and 14-17. In some aspects, the fusion protein comprises a CR1 protein or functional fragment thereof that does not contain SCRs 5-6, 11-13, and 18-30. In some aspects, the fusion protein comprises a CR1 protein or functional fragment thereof comprising SCRs 1-3, 7-10, and 14-17, and does not contain SCRs 5-6, 11-13, and 18-30.

In some aspects, the fusion protein comprises a CR1 protein or functional fragment thereof comprising SCRs 1-17. In some aspects, the fusion protein comprises a CR1 protein or functional fragment thereof that consists essentially of SCRs 1-17. In some aspects, the fusion protein comprises a CR1 protein or functional fragment thereof that does not contain SCRs 18-30. In some aspects, the fusion protein comprises a CR1 protein or functional fragment thereof comprising SCRs 1-17, and does not contain SCRs 18-30.

In some aspects, the fusion protein further comprises a complement receptor 2 (CR2) protein or a functional fragment thereof.

In some aspects, the CR2 protein or functional fragment thereof comprises CR2 SCRs 1-4. In some aspects, the CR2 protein or functional fragment thereof consists essentially of CR2 SCRs 1-4. In some aspects, the CR2 protein or functional fragment thereof comprises CR2 SCRs 1-4 and does not contain SCRs 5-15.

In some aspects, the CR2 protein or functional fragment thereof comprises CR2 SCRs 1-2. In some aspects, the CR2 protein or functional fragment thereof consists essentially of CR2 SCRs 1-2. In some aspects, the CR2 protein or functional fragment thereof comprises CR2 SCRs 1-2 and does not contain SCRs 3-15.

In some aspects, the fusion protein is a CR2-CR1 fusion protein disclosed herein.

In some aspects, the fusion protein comprises an amino acid sequence having at least 85%, at least 90%, at least 95%, at least 96%, at least 97%, at least 98%, at least 99%, or 100% identity to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some aspects, the fusion protein comprises an amino acid sequence having at least 85%, at least 90%, at least 95%, at least 96%, at least 97%, at least 98%, at least 99%, or 100% identity to SEQ ID NO: 12. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 12.

In some aspects, the delivery vector administered by suprachoroidal injection comprises a recombinant adeno-associated virus (rAAV) vector comprising a polynucleotide comprising a promoter operably linked to a nucleic acid encoding the fusion protein encapsidated by an AAV capsid.

In some aspects, the AAV capsid is an AAV2 serotype.

In some aspects, the polynucleotide comprises a nucleic acid sequence having at least 85%, at least 90%, at least 95%, at least 96%, at least 97%, at least 98%, at least 99%, or 100% identity to SEQ ID NO: 30. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 30.

In some aspects, the polynucleotide comprises a nucleic acid sequence having at least 85%, at least 90%, at least 95%, at least 96%, at least 97%, at least 98%, at least 99%, or 100% identity to SEQ ID NO: 78. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 78.

In some aspects, the polynucleotide comprises a nucleic acid sequence having at least 85%, at least 90%, at least 95%, at least 96%, at least 97%, at least 98%, at least 99%, or 100% identity to SEQ ID NO: 142. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 142.

In some aspects, the method is directed to administering a rAAV comprising a polynucleotide comprising a promoter operably linked to a nucleic acid encoding a CR2-CR1 fusion protein encapsidated by an AAV2 capsid, wherein the fusion protein comprises a complement receptor 1 (CR1) protein or a functional fragment thereof (e.g., a truncated CR1 protein) disclosed herein, wherein the administration is by suprachoroidal injection.

The optimal effective amount of the fusion protein, delivery vector, plasmid, nucleic acid, or pharmaceutical composition disclosed herein can be determined empirically and will depend on the type and severity of the disease, route of administration, disease progression and health, mass and body area of the individual. Such determinations are within the skill of one in the art. The effective amount can also be determined based on in vitro complement activation assays.

The fusion protein, delivery vector, plasmid, nucleic acid, or pharmaceutical composition disclosed herein may be administered in a single dose, or in multiple doses. The fusion protein, delivery vector, plasmid, nucleic acid, or pharmaceutical composition disclosed herein may be administered by injection in various locations in the eye or tissue associated with the eye, such as intraocular, intravitreal, subretinal, periocular, subconjunctival, choroid or sub-tenon. In some aspects, the fusion protein, the polynucleotide, the vector, or the AAV vector disclosed herein is administered to an eye of the subject by suprachoroidal injection.

### IX. Methods of Treatment

In some aspects, provided herein is a method of treating a subject suffering from a disease selected from AMD, macular degeneration, diabetic retinopathy, retinopathy of prematurity, macular dystrophy, retinal dystrophy, uveitis, keratitis, scleritis, retinitis pigmentosa, choroidal neovascularization, retinal neovascularization, glaucoma, and ocular inflammation, the method comprising administering any of the fusion proteins, the AAV vectors, the AAV capsids, or the pharmaceutical compositions disclosed herein.

In some aspects, provided herein is a method of treating a subject suffering from a disease selected from systemic lupus erythematosus (SLE), autoimmune arthritis, systemic inflammatory reaction syndrome, multiple organ dysfunction syndrome, ischemia-reperfusion syndrome, angioedema, capillary leak syndrome, hyperacute and acute graft rejection, vasculitis, nephritis, autoimmune disorders (e.g., SLE, rheumatoid arthritis, and myasthenia gravis), biomaterial incompatibility (e.g., following dialysis or cardiopulmonary bypass), severe trauma, burn, sepsis, Alzheimer's disease, multiple sclerosis, Guillain-Barre syndrome, Membranoproliferative glomerulonephritis (MPGN), hemolytic uremic syndrome (HUS) (e.g., atypical hemolytic uremic syndrome (aHUS)), Hereditary angioedema (e.g., type I or type II), paroxysmal nocturnal hemoglobinuria (PNH), periodontitis, asthma, Chronic obstructive pulmonary disease, Acute respiratory distress syndrome, Crohn's disease, artherosclerosis, Osteoarthritis, Bullous pemphigoid, psoriasis, Hidradenitis suppurativa, C3 glomerulopathy, Lupus nephritis, IgA nephropathy, MN, myocardial infarction, Parkinson's disease, schizophrenia, Neuromyelitis optica, or multiple sclerosis, the method comprising administering any of the fusion proteins, the AAV vectors, the AAV capsids, or the pharmaceutical compositions disclosed herein.

In some aspects, the fusion protein, the AAV vector, the AAV capsid, or the pharmaceutical composition is administered to an eye of the subject using intravitreal or subretinal injection. In some aspects, the fusion protein, the polynucleotide, the vector, the AAV vector, or pharmaceutical composition disclosed herein is administered to an eye of the subject using suprachoroidal injection.

In some aspects, the fusion protein, the AAV vector, the AAV capsid, or the pharmaceutical composition is administered to eye tissue, wherein the eye tissue comprises retinal pigment epithelium (RPE) or choroid.

In some aspects, the macular degeneration comprises age-related macular degeneration (AMD). In some aspects, the AMD is dry AMD. In some aspects, the AMD is wet AMD.

In some aspects, the AMD is an advanced form (e.g., geographic atrophy).

Geographic atrophy (GA) is an advanced form of age-related macular degeneration (AMD) in which atrophic lesions progressively degenerate the photoreceptors, retinal pigment epithelium (RPE), and underlying choriocapillaris, ultimately resulting in irreversible vision loss. While disease progression and lesion growth are largely variable across patients, lesions typically appear first in the extrafoveal space (non-central GA). Resultant formation of scotomas, or blind spots, outside the fovea do not affect central vision, but impair visual functions such as reading ability, facial recognition, and contrast sensitivity. These lesions are reported to progress significantly faster than foveal lesions, with directional progression towards the periphery almost 3 times faster than to the fovea (Lindner, M., et al. (2015). Ophthalmology 122(7): 1356-1365; Fleckenstein, M., et al. (2018). Ophthalmology 125(3): 369-390.). Gradually over time, lesions expand to the macula and fovea (central GA), and large central scotomas are formed, leading to severe loss of visual acuity and legal blindness (Boyer, D. S., et al. (2017). Retina 37(5): 819-835; Bakri, S. J., et al. (2023). J Manag Care Spec Pharm 29(5-a Suppl): S2-s11). The median time of progression to legal blindness for GA patients is estimated at 6.2 years (Chakravarthy, U., et al. (2018). Ophthalmology 125(6): 842-849). The formation of atrophic lesions distinguishes GA (dry AMD) from neovascular AMD (nAMD or wet AMD), the other late-stage form in which blood vessels leak fluid such as blood and serum into the macula, resulting in severe, rapid vision loss (Deng, Y., et al. (2022). Genes Dis 9(1): 62-79).

GA affects over 5 million people globally (Fleckenstein, M., et al. (2018). Ophthalmology 125(3): 369-390). Incidence increases drastically with age, where an estimated 1 in 29 adults over 75 years old have geographic atrophy rising to 1 in 4 adults over 90 years old (Klein, R., et al. (1993). Ophthalmology 100(3): 406-414; Vingerling, J. R., et al. (1995). Ophthalmology 102(2): 205-210; Hirvelä, H., et al. (1996). Ophthalmology 103(6): 871-877; Sunness, J. S. (1999). Mol Vis 5: 25). Globally, overall prevalence of GA (all ages) ranges from 0.14 to 1.11%, and is higher in Europeans than in Asians, Africans, and Hispanics (Wong 2014). While age is the primary risk factor, environmental and genetic factors including cigarette smoking, higher body mass index, hyperlipidemia, hypertension, familial history, and certain single nucleotide polymorphisms (SNPs) additionally contribute to the risk of developing AMD and subsequent GA (Clemons 2005, Shahid 2012, Velilla 2013, Fleckenstein 2018, Thomas 2021).

The pathophysiology of GA is attributed to RPE damage resulting from various environmental, physiologic, and genetic factors. With aging, transport of metabolic waste from the photoreceptors to the choroid by the RPE become inefficient, and cellular debris, protein, and lipid fragments accumulate between the RPE and in Bruch's membrane, leading to the formation of drusen (Boyer, D. S., et al. (2017). Retina 37(5): 819-835). Excessive drusen buildup, which is further accelerated by environmental oxidative stressors such as smoking and sunlight exposure, is thought to trigger inflammation via the complement cascade. Mutations in genes responsible for the regulation of the complement cascade, such as complement factor H, lead to dysregulation of the complement system and are strongly linked to development of AMD (Shughoury, A., et al. (2022). Genes (Basel) 13(7)). Overactivation of the complement system results in degradation of individual retinal layers (photoreceptors, RPE, and choriocapillaris) by phagocytosis and membrane attack complex (MAC)-assisted cell death, creating space for atrophic lesion growth and decreasing choroid flow (Sastre-Ibanez, M., et al. (2018). Arch Soc Esp Oftalmol (Engl Ed) 93(1): 22-34; Bakri, S. J., et al. (2023). J Manag Care Spec Pharm 29(5-a Suppl): S2-s11).

Characteristics of GA lesions, including baseline lesion size, are highly indicative of disease progression rate (Bakri, S. J., et al. (2023). J Manag Care Spec Pharm 29(5-a Suppl): S2-s11). As such, clinical disease management is based largely on multimodal imaging, where GA lesions can be distinguished as round or oval shapes with sharp margins (Bird, A. C., et al. (1995). Surv Ophthalmol 39(5): 367-374). Fundus autofluorescence (FAF), which detects cell death or damage based on decreased autofluorescence of RPE cells, is the gold standard for monitoring GA progression (Sacconi, R., et al. (2017). Ophthalmol Ther 6(1): 69-77). Additionally, specific patterns in FAF can be used as prognostic markers of disease progression rate (Holz, F. G., et al. (2007). Am J Ophthalmol 143(3): 463-472). Optical coherence tomography (OCT) detects structural changes in retinal layers and is the recommended reference method for defining stages of GA due to collection of 3-dimensional measurements, as well as ease of image acquisition (Sadda, S. R., et al. (2018). Ophthalmology 125(4): 537-548). Fundus photography has also been traditionally used to identify GA lesions, as identified by round or oval shapes of pigmentation with sharp margins (Sacconi, R., et al. (2017). Ophthalmol Ther 6(1): 69-77), though this technique lacks the ability to thoroughly characterize lesions. Both OCT and FAF allow for quantitative morphological assessment of GA lesions, and high correlation has been shown between the two methods (Garzone, D., et al. (2022). Sci Rep 12(1): 21911). Beyond lesion size, OCT can also measure RPE loss and features of PR degeneration including loss of the ellipsoid zone (EZ) and absence of the external limiting membrane, foveal center involvement, the size of drusen, and the presence of reticular pseudodrusen (Kalra, G., et al. (2022). J Pers Med 13(1); Schmidt-Erfurth, U., et al. (2023). Ophthalmologie 120(9): 965-969). Moreover, while best-corrected visual acuity (BCVA) is a recognized standard to assess vision, it does not correlate well with GA progression as foveal-sparing lesions do not affect central vision; therefore, individual letter reading may remain unimpaired until lesions reach the fovea (Sunness, J. S. (1999). Mol Vis 5: 25).

In some aspects, provided herein is a use of any of the fusion proteins, the AAV vectors, the AAV capsids, or the pharmaceutical compositions disclosed herein, for the treatment of a subject suffering from a complement-mediated disorder selected from: (i) various disorders characterized by hemolysis or hemolytic anemia such as atypical hemolytic uremic syndrome, cold agglutinin disease, paroxysmal nocturnal hemoglobinuria, transfusion reactions; (ii) transplant rejection (e.g., hyperacute or acute transplant rejection) or transplant dysfunction; (iii) disorders involving ischemia/reperfusion injury such as trauma, surgery (e.g., aneurysm repair), myocardial infarction, ischemic stroke; (iv) disorders of the respiratory system such as asthma and chronic obstructive pulmonary disease (COPD); (v) arthritis, e.g., rheumatoid arthritis; or (vi) ocular disorders such as age-related macular degeneration (AMD), diabetic retinopathy, glaucoma, and uveitis.

In some aspects, provided herein is a method of treating a subject suffering from a) a solid tumor associated with angiogenesis or b) an ocular disease or condition associated with abnormal blood vessel in an eye, comprising administering to the subject an effective amount of any of the fusion proteins disclosed herein (e.g., a CR1-VID fusion protein), any of the nucleic acids disclosed herein, any of the vectors disclosed herein, or any of the AAV vectors disclosed herein. In some aspects, the ocular disease or condition associated with abnormal blood vessel in an eye is diabetic retinopathy. In some aspects, the ocular disease or condition associated with abnormal blood vessel in an eye is wet age-related macular degeneration. In some aspects, the subject has breast cancer, colorectal cancer, lung cancer, kidney cancer, gastric cancer, ovarian cancer, or retinoblastoma.

In some aspects, the methods disclosed here result in a decrease in the ratio of C3b:iC3b protein levels. In some aspects, the ratio of C3b:iC3b is reduced in the subject (e.g., the eye of the subject) after the administration any of the fusion proteins disclosed herein (e.g., a CR2-CR1 fusion protein), any of the nucleic acids disclosed herein, any of the vectors disclosed herein, any of the AAV vectors disclosed herein, or any of the pharmaceutical compositions disclosed herein.

In some aspects, the methods disclosed here result in a decrease in C3b protein levels. In some aspects, C3b is reduced in the subject (e.g., the eye of the subject) after the administration any of the fusion proteins disclosed herein (e.g., a CR2-CR1 fusion protein), any of the nucleic acids disclosed herein, any of the vectors disclosed herein, any of the AAV vectors disclosed herein, or any of the pharmaceutical compositions disclosed herein.

In some aspects, the methods disclosed here result in an increase in inactive C3b (iC3b) protein levels. In some aspects, iC3b is increased in the subject (e.g., the eye of the subject) after the administration any of the fusion proteins disclosed herein (e.g., a CR2-CR1 fusion protein), any of the nucleic acids disclosed herein, any of the vectors disclosed herein, any of the AAV vectors disclosed herein, or any of the pharmaceutical compositions disclosed herein.

Methods for detecting C3b and iC3b protein levels can include densitometry and Western blot analysis with anti-C3b antibodies and anti-iC3b antibodies. In some aspects, the C3b and iC3b can evaluated individually. In some aspects, the C3b decreases slightly and the iC3b increases. In some aspects, the decrease in the ratio of C3b:iC3b protein levels can bring the individual assessments closer to normal levels. In some aspects, the method results in reduction in C3b generation by inhibiting C3 convertase. In some aspects, the CR1 acts as a cofactor for the conversion of C3b into iC3b, e.g., accelerating C3b's decay and increasing iC3b.

Certain aspects of the disclosure are directed to a method of treating a subject suffering macular degeneration comprising administering a fusion protein, a delivery vector comprising a nucleic acid encoding the fusion protein (e.g., an AAV vector), or pharmaceutical composition comprising the fusion protein or the delivery vector to a subject in need thereof, wherein the fusion protein comprises a complement receptor 1 (CR1) protein or a functional fragment thereof (e.g., a truncated CR1 protein) disclosed herein. In some aspects, the fusion protein, a delivery vector comprising a nucleic acid encoding the fusion protein (e.g., an AAV vector), or pharmaceutical composition is administered by suprachoroidal injection.

In some aspects, the macular degeneration comprises age-related macular degeneration (AMD). In some aspects, the AMD is dry AMD. In some aspects, the AIMD is wet AMD. In some aspects, the AMD is an advanced form (e.g., geographic atrophy).

In some aspects, the fusion protein comprises a CR1 protein or functional fragment thereof comprising Short Consensus Repeats (SCRs) 1-4, 7-11, and 14-17. In some aspects, the fusion protein comprises a CR1 protein or functional fragment thereof that consists essentially of SCRs 1-4, 7-11, and 14-17. In some aspects, the fusion protein comprises a CR1 protein or functional fragment thereof that does not contain SCRs 5-6, 12-13, and 18-30. In some aspects, the fusion protein comprises a CR1 protein or functional fragment thereof comprising SCRs 1-4, 7-11, and 14-17, and does not contain SCRs 5-6, 12-13, and 18-30.

In some aspects, the fusion protein comprises a CR1 protein or functional fragment thereof comprising SCRs 1-14. In some aspects, the fusion protein comprises a CR1 protein or functional fragment thereof that consists essentially of SCRs 1-14. In some aspects, the fusion protein comprises a CR1 protein or functional fragment thereof does not contain SCRs 15-30. In some aspects, the fusion protein comprises a CR1 protein or functional fragment thereof comprising SCRs 1-14, and does not contain SCRs 15-30.

In some aspects, the fusion protein comprises a CR1 protein or functional fragment thereof that comprises SCRs 1-3, 7-10, and 14-17. In some aspects, the fusion protein comprises a CR1 protein or functional fragment thereof that consists essentially of SCRs 1-3, 7-10, and 14-17. In some aspects, the fusion protein comprises a CR1 protein or functional fragment thereof that does not contain SCRs 5-6, 11-13, and 18-30. In some aspects, the fusion protein comprises a CR1 protein or functional fragment thereof comprising SCRs 1-3, 7-10, and 14-17, and does not contain SCRs 5-6, 11-13, and 18-30.

In some aspects, the fusion protein comprises a CR1 protein or functional fragment thereof comprising SCRs 1-17. In some aspects, the fusion protein comprises a CR1 protein or functional fragment thereof that consists essentially of SCRs 1-17. In some aspects, the fusion protein comprises a CR1 protein or functional fragment thereof that does not contain SCRs 18-30. In some aspects, the fusion protein comprises a CR1 protein or functional fragment thereof comprising SCRs 1-17, and does not contain SCRs 18-30.

In some aspects, the fusion protein further comprises a complement receptor 2 (CR2) protein or a functional fragment thereof. In some aspects, the fusion protein is a CR2-CR1 fusion protein disclosed herein.

In some aspects, the fusion protein comprises an amino acid sequence having at least 85%, at least 90%, at least 95%, at least 96%, at least 97%, at least 98%, at least 99%, or 100% identity to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some aspects, the fusion protein comprises an amino acid sequence having at least 85%, at least 90%, at least 95%, at least 96%, at least 97%, at least 98%, at least 99%, or 100% identity to SEQ ID NO: 12. In some aspects, the fusion protein comprises an amino acid sequence having at least about 85%, at least about 90%, at least about 95%, at least about 96%, at least about 97%, at least about 98%, at least about 99%, or 100% identity to SEQ ID NO: 12.

In some aspects, the delivery vector administered by suprachoroidal injection comprises a recombinant adeno-associated virus (rAAV) vector comprising a polynucleotide comprising a promoter operably linked to a nucleic acid encoding the fusion protein encapsidated by an AAV capsid.

In some aspects, the AAV capsid is an AAV2 serotype.

In some aspects, the polynucleotide comprises a nucleic acid sequence having at least 85%, at least 90%, at least 95%, at least 96%, at least 97%, at least 98%, at least 99%, or 100% identity to SEQ ID NO: 30. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 30.

In some aspects, the polynucleotide comprises a nucleic acid sequence having at least 85%, at least 90%, at least 95%, at least 96%, at least 97%, at least 98%, at least 99%, or 100% identity to SEQ ID NO: 78. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 78.

In some aspects, the polynucleotide comprises a nucleic acid sequence having at least 85%, at least 90%, at least 95%, at least 96%, at least 97%, at least 98%, at least 99%, or 100% identity to SEQ ID NO: 142. In some aspects, the polynucleotide comprises a nucleic acid sequence having at least about 85%, at least about 90%, at least about 95%, at least about 96%, at least about 97%, at least about 98%, at least about 99%, or 100% identity to SEQ ID NO: 142.

In some aspects, the method is directed to treating a subject suffering macular degeneration (e.g., geographic atrophy) comprising administering a rAAV comprising a polynucleotide comprising a promoter operably linked to a nucleic acid encoding a CR2-CR1 fusion protein encapsidated by an AAV2 capsid, wherein the fusion protein comprises a complement receptor 1 (CR1) protein or a functional fragment thereof (e.g., a truncated CR1 protein) disclosed herein, wherein the administration is by suprachoroidal injection.

The following examples are illustrative and do not limit the scope of the claimed aspects.

### EXAMPLES

### Example 1. Exemplary CR2-CR1 Fusion Protein Constructs

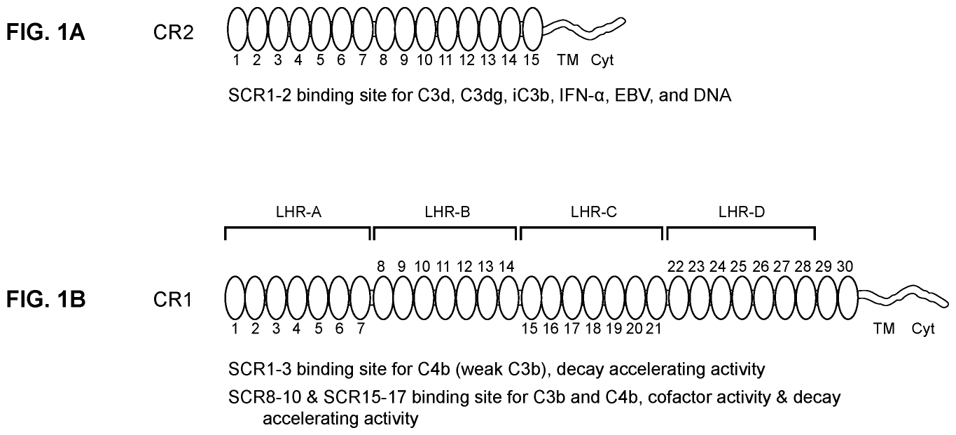

Complement Receptor 2 (“CR2”) (FIG. 1A) contains 15 short consensus repeats (“SCR”), with SCR1 and SCR2 containing binding sites for C3d, C3dg, iC3b, CD23, IFN-α, Epstein-Barr Virus, and various forms of DNA. Complement Receptor 1 (“CR1”) (FIG. 1B) contains 30 SCRs. CR1 SCR1-3 contains a binding site for C4b and a weak binding site for C3b (“site 1”), which provides decay accelerating activity. CR1 SCR8-10 and SCR15-17 contain binding sites (“site 2”) for C3b and C4b, which provides cofactor activity and decay accelerating activity.

CR2-CR1 fusion proteins were designed to combine CR2's ability to bind to the site of complement activation (i.e., iC3b, C3dg, and C3d) and CR1's cofactor activity and CR1's ability to inhibit the complement pathway by preventing the assembly of C3 convertase on the cell surface and accelerating its decay. Because the AAV vector genome is limited to approximately 4.7 kb, it is not possible to package a fusion protein containing the full length CR2 and CR1 proteins. Therefore, expression cassettes were designed to express novel fusion proteins containing truncated CR2 and CR1 domains. Table 1 and Table 2 show the details of these constructs.

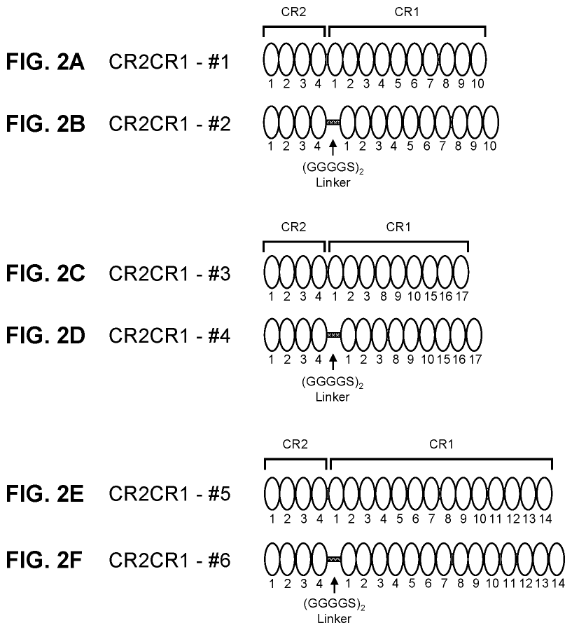

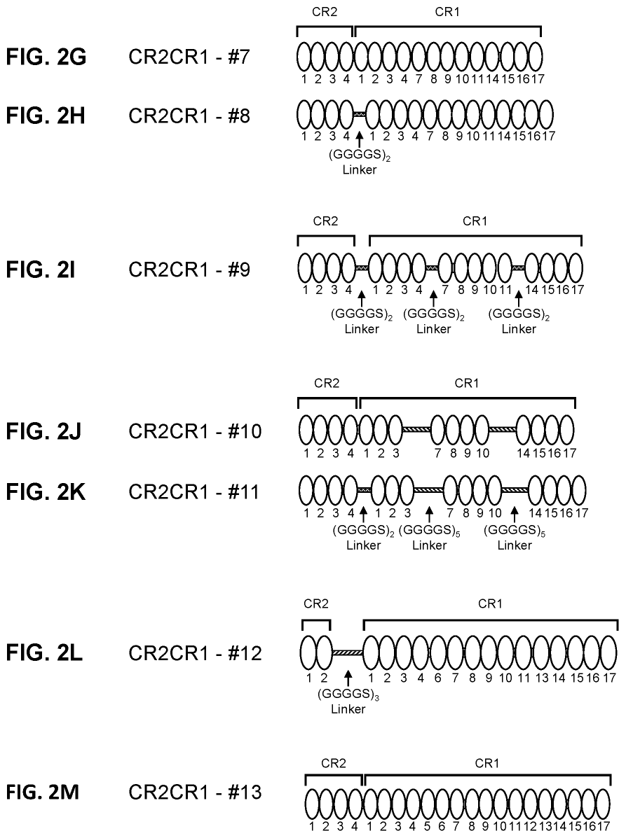

TABLE 1
CR2-CR1 Fusion Protein Expression Cassette Details

| | | | Poly A | AAV Cargo | Protein | Predicted |
|---|---|---|---|---|---|---|
| Protein Name | Plasmids | Promoter | Tail | Size (Kb) | length (AA) | M.W. (kDa) |
| CR2CR1 #1 | TS-2101-TS-2103 | smCBA | bGH | 4.27 | 901 | 98.6 |
| (FIG. 2A) | | | | | | |
| CR2CR1 #1* | TS-2137 | smCBA | Synthetic | 4.08 | 898 | 98.3 |
| CR2CR1 #2 | TS-2104-TS-2106 | smCBA | bGH | 4.3 | 911 | 99.2 |
| (FIG. 2B) | | | | | | |
| CR2CR1 #3 | TS-2107-TS-2109 | smCBA | bGH | 4.04 | 826 | 90.6 |
| (FIG. 2C) | | | | | | |
| CR2CR1 #4 | TS-2110-TS-2112 | smCBA | bGH | 4.07 | 836 | 91.2 |
| (FIG. 2D) | | | | | | |
| CR2CR1 #5 | TS-2113-TS-2115 | smCBA | bGH | 5.02 | 1153 | 126.2 |
| (FIG. 2E) | | | | | | |
| CR2CR1 #6 | TS-2116-TS-2118 | smCBA | bGH | 5.07 | 1163 | 126.8 |
| (FIG. 2F) | | | | | | |
| CR2CR1 #7 | TS-2119-TS-2121 | smCBA | Synthetic | 4.69 | 1102 | 120.8 |
| (FIG. 2G) | | | | | | |
| CR2CR1 #8 | TS-2122-TS-2124 | smCBA | Synthetic | 4.72 | 1112 | 121.4 |
| (FIG. 2H) | | | | | | |
| CR2CR1 #9 | TS-2125-TS-2127 | smCBA | Synthetic | 4.78 | 1132 | 122.7 |
| (FIG. 2I) | | | | | | |
| CR2CR1 #10 | TS-2128-TS-2130 | smCBA | Synthetic | 4.63 | 1080 | 113.2 |
| (FIG. 2J) | | | | | | |
| CR2CR1 | TS-2131-TS-2133 | smCBA | Synthetic | 4.66 | 1090 | 113.8 |
| #11(FIG. 2K) | | | | | | |
| CR2CR1 #12 | TS-2134-TS-2136 | smCBA | Synthetic | 4.77 | 1128 | 122.6 |
| (FIG. 2L) | | | | | | |
| CR2CR1 #13 | TS-2138 | smCBA | Synthetic | 5.43 | 1348 | 147.6 |
| (FIG. 2M) | | | | | | |

*The protein expressed from TS-2137 has a 3 amino acid deletion from the C-terminus of CR1 SCR10 relative to canonical CR2CR1-#1. No functional difference was observed.

TABLE 2
CR2-CR1 Fusion Construct Spacer Design

| | | | | | | CR2- | | |
|---|---|---|---|---|---|---|---|---|
| | Site 1 | Site 2 | CR2 | CR1 | Total # | CR1 | Spacing between | |
| Protein | Count | Count | SCRs | SCRs | SCRs | Linker | CR1 active sites | Constructs |
| CR2CR1 #1 | 1 | 1 | SCR1-4 | SCR1-10 | 14 | — | Native (4 SCRs) | FIG. 2A |
| CR2CR1 #1 | 1 | 1 | SCR1-4 | SCR1-10 | 14 | — | Native (4 SCRs) | FIG. 2M |
| CR2CR1 #2 | 1 | 1 | SCR1-4 | SCR1-10 | 14 | (G4S)2 | Native (4 SCRs) | FIG. 2B |
| CR2CR1 #3 | 1 | 2 | SCR1-4 | SCR1-3, | 13 | — | None | FIG. 2C |
| | | | | SCR8-10, | | | | |
| | | | | SCR15-17 | | | | |
| CR2CR1 #4 | 1 | 2 | SCR1-4 | SCR1-3, | 13 | (G4S)2 | None | FIG. 2D |
| | | | | SCR8-10, | | | | |
| | | | | SCR15-17 | | | | |
| CR2CR1 #5 | 1 | 1 | SCR1-4 | SCR1-14 | 18 | — | Native (4 SCRs) | FIG. 2E |
| CR2CR1 #6 | 1 | 1 | SCR1-4 | SCR1-14 | 18 | (G4S)2 | Native (4 SCRs) | FIG. 2F |
| CR2CR1 #7 | 1 | 2 | SCR1-4 | SCR1-4, | 17 | — | 2 SCRs | FIG. 2G |
| | | | | SCR7-11, | | | | |
| | | | | SCR14-17 | | | | |
| CR2CR1 #8 | 1 | 2 | SCR1-4 | SCR1-4, | 17 | (G4S)2 | 2 SCRs | FIG. 2H |
| | | | | SCR7-11, | | | | |
| | | | | SCR14-17 | | | | |
| CR2CR1 #9 | 1 | 2 | SCR1-4 | SCR1-4, | 17 | — | SCR + (G4S)2 + | FIG. 2I |
| | | | | SCR7-11, | | | SCR | |
| | | | | SCR14-17 | | | | |
| CR2CR1 #10 | 1 | 2 | SCR1-4 | SCR1-3, | 15 | — | (G4S)12 + SCR | FIG. 2J |
| | | | | SCR7-10, | | | | |
| | | | | SCR14-17 | | | | |
| CR2CR1 #11 | 1 | 2 | SCR1-4 | SCR1-3, | 15 | (G4S)2 | (G4S)12 + SCR | FIG. 2K |
| | | | | SCR7-10, | | | | |
| | | | | SCR14-17 | | | | |
| CR2CR1 #12 | 1 | 2 | SCR1-2 | SCR1-4, | 17 | (G4S)6 | 3 SCRs | FIG. 2L |
| | | | | SCR6-12, | | | | |
| | | | | SCR14-17 | | | | |
| CR2CR1 #13 | 1 | 2 | SCR1-4 | SCR1-17 | 21 | — | Native (4 SCRs) | FIG. 2M |

### Example 2. CR2-CR1 Fusion Construct In Vitro Expression

To test the mRNA expression of the constructs generated in Example 1, the plasmids expressing the CR2-CR1 fusion proteins were transfected in HEK293 cells (6-well plate, 72 hour transfection, 2.5 μg plasmid/well). Cells were seeded in cell culture-treated 48-well plates in regular media with 10% Fetal Bovine Serum and 1% Penicillin Streptomycin at 40-75% confluency depending on cell type and cell doubling. 24 hours after seeding, when cells were 80-90% confluent, cell media was exchanged for 300 μL fresh media per well, and plasmids to be transfected were diluted in OptiMEM and incubated with Lipofectamine 3000 (Invitrogen, Waltham, MA, USA) according to the manufacturer's instructions. Diluted plasmids and Lipofectamine mix were added to cell culture in triplicate at three concentrations (0.83 μg/mL, 0.42 μg/mL, and 0.21 μg/mL per well; reported as 250 ng, 125 ng, and 62.5 ng/well in a 48 well plate). After 72 hours, supernatants were collected and cells were washed for RNA extraction with PBS three times. Both media supernatant and the cells were stored at −80° C. until further analyzed or processed.

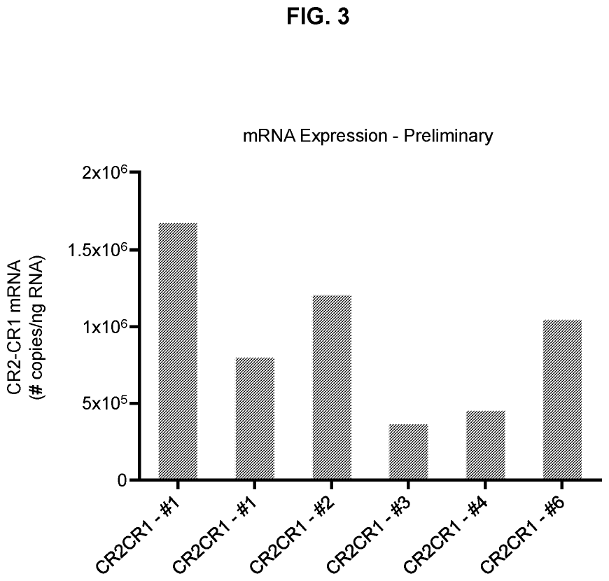

A RT-qPCR assay was used to measure mRNA expression (# copies/ng RNA). CR2-CR1 RT-qPCR assay targeted the junction between CR2 and CR1. The targeted sequence was present in all plasmids except those for FIG. 2L. Transgenic gene junction was not present in human genomic DNA or mRNA. See FIG. 3.

mRNA Quantitation

Total RNA was isolated from transfected/transduced cells using the Zymo Quick-RNA 96 Kit (Zymo catalog #R1052) following the manufacturer's instructions. Isolated RNA was then subjected to DNase treatment/purification using the RNA Clean & Concentrator-96 Kit (Zymo catalog #R1080) following the manufacturer's instructions. The concentration of isolated RNA was measured via UV/VIS spectroscopy using the NanoDropOne instrument (Fisher catalog #13-400-518). Transgenic RNA was quantified via 1-step reverse transcription quantitative PCR (RT-qPCR) using a 5′ endonuclease activity assay. The RT-qPCR assay targeted the junction region between a modified human Complement Receptor 2 (CR2) and a modified human Complement Receptor 1 (CR1) present in transgenic hCR1-CR2 mRNA. Restriction enzyme linearized plasmid DNA containing the hCR2-hCR1 Junction DNA sequence was used as a quantification standard for this RT-qPCR assay.

All primer RT-qPCR were purchased from Integrated DNA Technologies.

CR2-CR1 J Forward:
(SEQ ID NO: 74)
5′ GACAGGGAGTTGCTTGGAC 3′
CR2-CR1 J Reverse:
(SEQ ID NO: 75)
5′ GGGAAACTCAAATTCATCAGTTAGG 3′
CR2-CR1 J Probe:
(SEQ ID NO: 76)
5′ CAGAATGGCTTCCATTTGCCAGGC 3′.

76). Probe modifications 5′ 6-FAM (Fluorescein), 3′ ZEN/Iowa Black fluorescence quenchers.

TaqMan™ Fast Virus 1-Step Master Mix (ThermoFisher catalog #4444434) was used to create 1-step RT-qPCR master mix. QuantStudio 5 (ThermoFisher catalog #A28568) PCR platform was used for RT-qPCR, using “Fast” instrument settings. Thermocycling conditions were as follows:
1) 50° C. for 5 minutes
2) 95° C. for 20 seconds
3) 95° C. for 3 seconds
4) 60° C. for 30 seconds
Repeat steps 3-4 39 times.

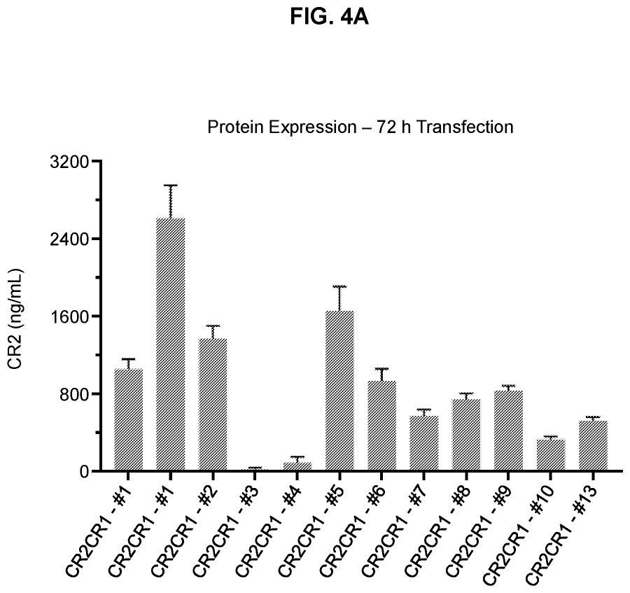

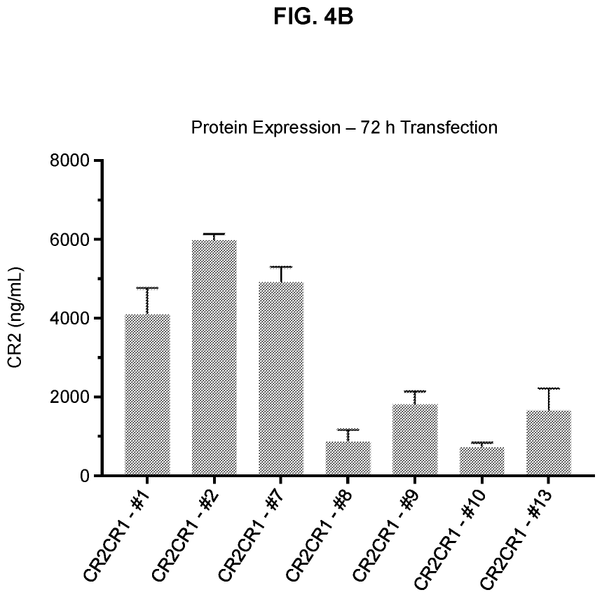

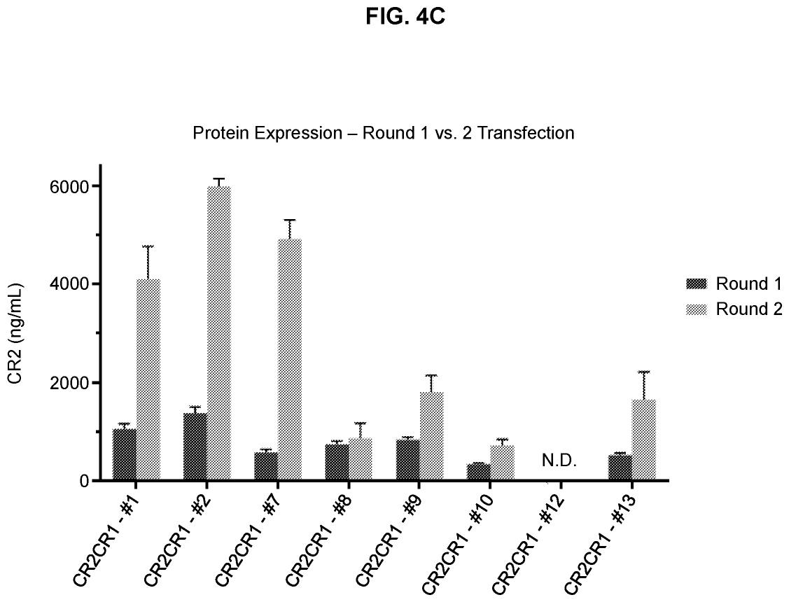

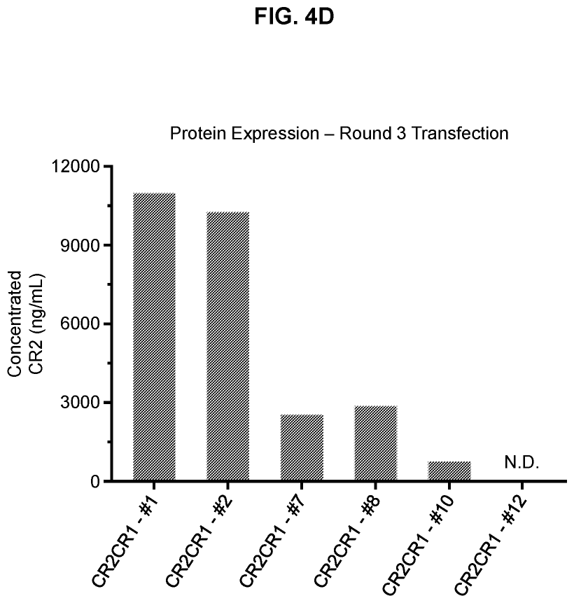

Next, the protein expression of the constructs were analyzed using a Gryos® Immunoassay. HEK293 cells were transfected with 2.5 μg plasmid/well or 3 μg plasmid/well (6-well plate, 72 hour transfection, n=3). See FIGS. 4A-4D. Protein detection used an antibody pair able to detect SCR1-4 of CR2-CR1 constructs

Expressed secreted protein was detected from all constructs except TS-2135 expressing fusion protein CR2CR1 #12, likely due to the assay's inability to detect the shorter CR2 domain contained in this construct.

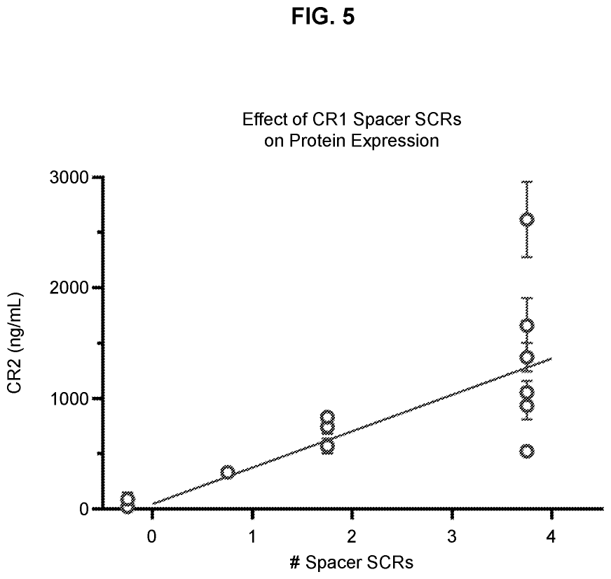

Reduction of spacer SCRs appeared to lead to decreased protein expression level. See FIG. 5 CR2-CR1 fusion protein quantification in cell culture supernatant.

The CR2-derived protein region is identical between all CR2-CR1 fusion proteins, so the concentration of secreted CR2-CR1 fusion protein in cell culture supernatant was quantified using a three-step (capture-analyte-detection) immunoassay specific for SCR1-4 of the CR2 protein. All analyses were performed on a Gyrolab xPlore system (Gyros Protein Technologies) with Bioaffy 4000 CDs (Gyros Protein Technologies, method 4000-3W-007-X). Anti-CR2 antibody (Atlas Antibodies) was biotinylated at a 12:1 labeling ratio by Gyros Protein Technologies, and a noncompeting anti-CR2 antibody (Thermo Scientific) was labeled with AlexaFluor647 fluorophore using an APEX Antibody Labeling kit (Thermo Scientific). A standard curve (0.6-2400 ng/mL) was generated using recombinant human CR2 protein (R&D Systems) diluted in Rexxip CCS buffer, and cell culture supernatant samples were diluted 1:5-1:500 in Rexxip CCS as needed. The concentration of CR2 SCR1-4-containing protein in cell culture supernatant was determined by comparison of sample signal at 1% PMT to the standard curve signal fit to a five-parameter logistic curve using the Gyrolab Evaluator software (Gyros Protein Technologies).

### Example 3. CR2-CR1 Fusion Constructs In Vitro Activity

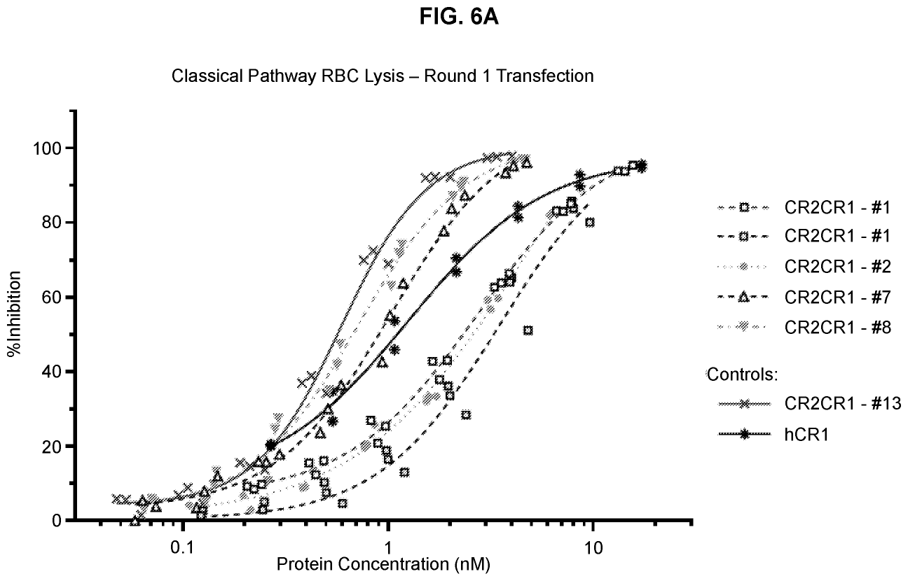

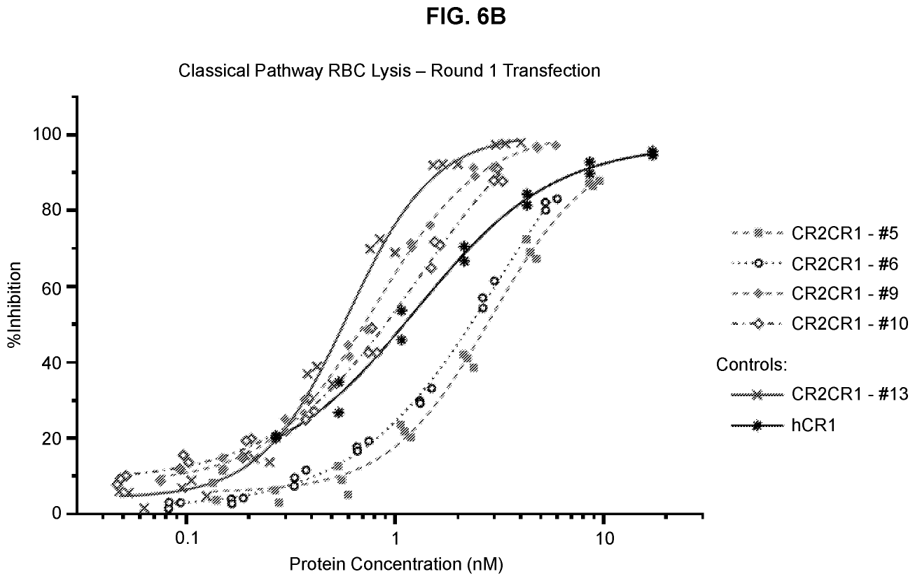

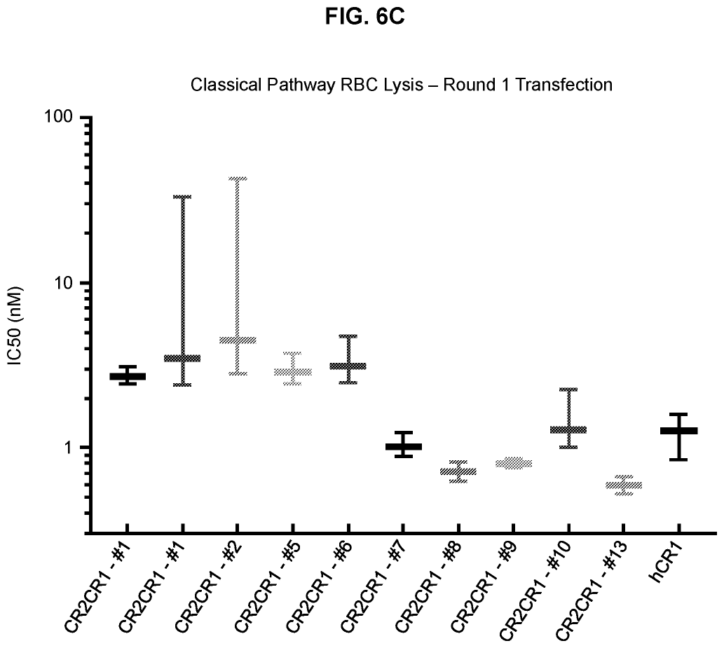

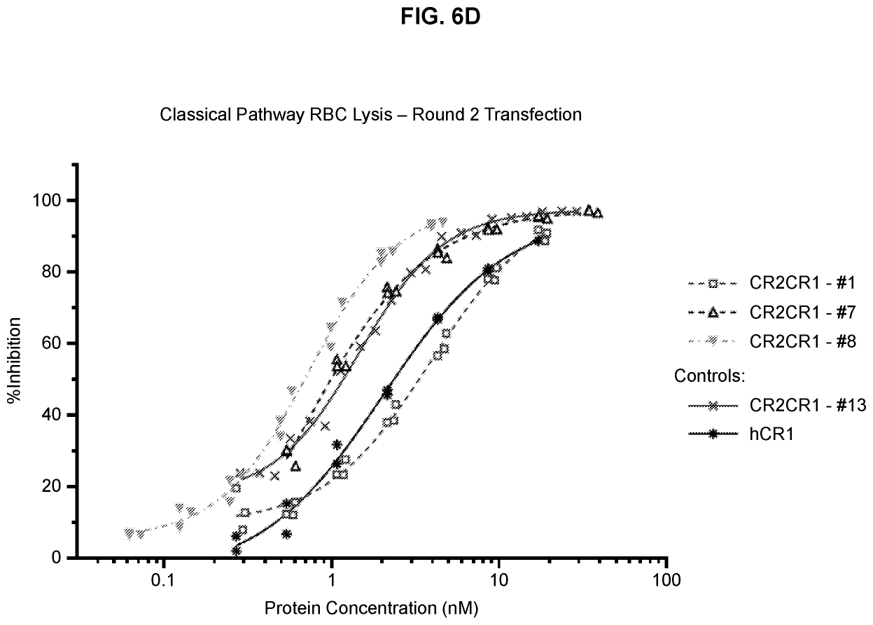

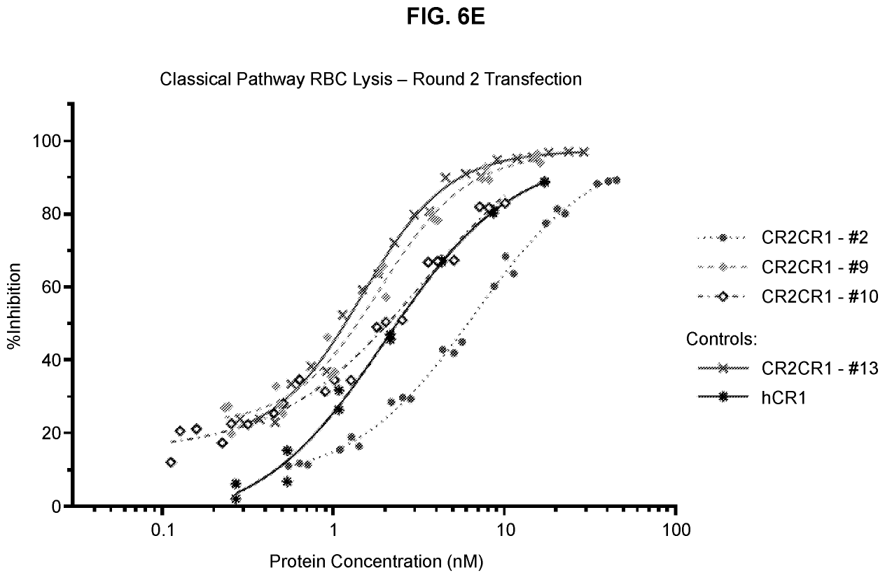

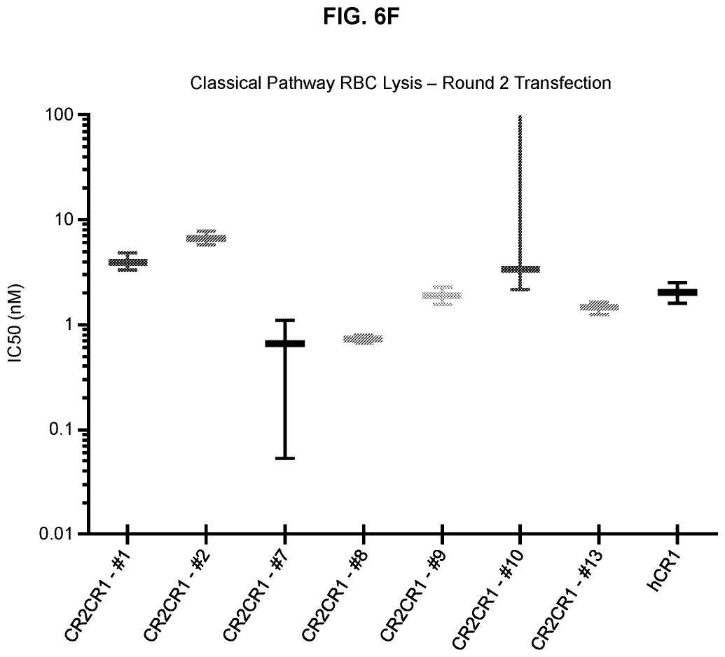

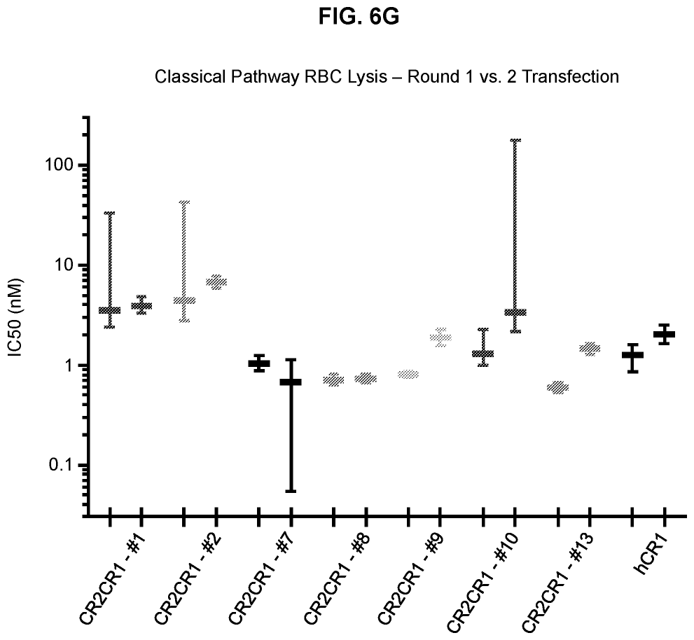

To test the inhibitory activity of the CR2-CR1 fusion constructs of Example 1 on the classical complement pathway, a red blood cell lysis assay was used (sensitized sheep red blood cell lysis with human serum; HEK293 cells, 6-well plate, 72 hour transfection, 2.5 μg plasmid/well or 3 μg plasmid/well, n=3, Graphpad Prism 4PL calc. IC₅₀ with 95% Confidence Interval). See FIGS. 6A-6G.

### Sample Concentration for Functional Assessment

Prior to functional assay use, all transfection cell culture supernatant samples were concentrated to increase in-assay dynamic range. Samples were concentrated with 50 kDa molecular weight cut-off centrifugal filters (Millipore) and then brought up to the appropriate volume in PBS for a final 5-fold concentration.

### Human Classical RBC Lysis Assay

Transfection cell culture supernatant samples were screened for the inhibition of the human classical complement pathway using a cell lysis assay that employs Rabbit Sensitized Sheep Red Blood Cells (RBCs) to activate functional complement components present in Normal Human Serum (NHS). The terminal components of the activated complement pathway will lyse the RBCs, and the amount of lysis (measured by absorbance) is used to directly determine the amount of complement activity. Human Recombinant CR1 (R&D Systems) served as an internal positive lysis inhibition control and a concentrated mock transfection sample was used as a background lysis control. To calculate sample percent inhibition, EDTA and NHS (Complement Technology, Inc.) were used as the 100% lysis inhibition and 0% lysis inhibition values, respectively. Concentrated samples and internal controls were serially diluted 1:2 in 40 μL ice cold GVB++ buffer (0.1% gelatin, 5 mM Veronal, 145 mM NaCl, 0.025% NaN3, pH 7.3; with 0.15 mM CaCl2) and 0.5 mM MgCl2, Complement Technology, Inc) in a 96-well, clear, V-bottom assay plate (Corning). RBCs (Complement Technology, Inc) were washed twice with fresh GVB++ and diluted in ice cold GVB++ to reach a final working concentration of 3.75E+08 cells per mL. A 40 μL volume of NHS diluted 1:50 in ice cold GVB++ followed by 40 μL of diluted RBCs were added to the appropriate wells. The assay plate was incubated at 37° C. for 30 minutes and then centrifuged at 500×g, 4° C. for 5 minutes. After pelleting the RBCs, 100 μL of supernatant from each well was transferred to a 96-well, clear, flat-bottomed read plate. The end-point absorbance at 412 nm was read using a spectrophotometer (Molecular Devices) and the percent inhibition and EC50 for each sample dilution series was calculated.

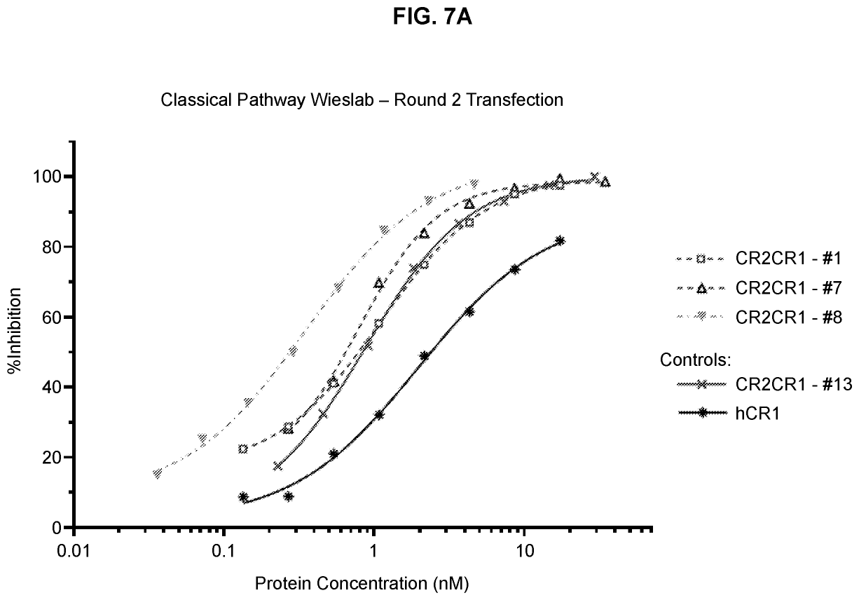

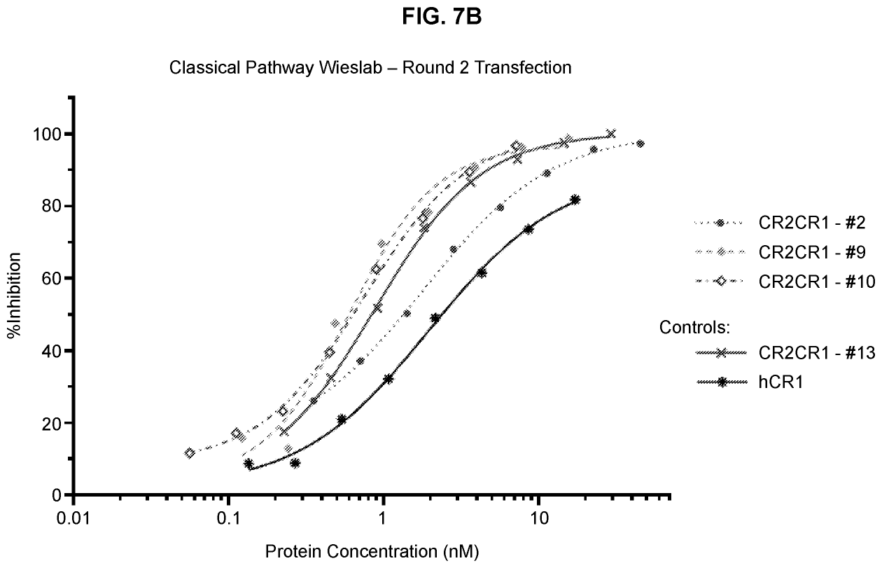

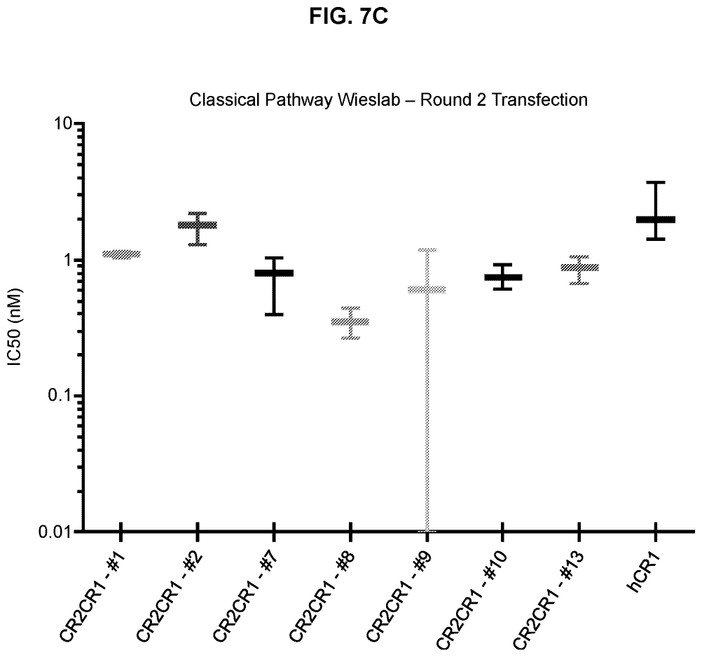

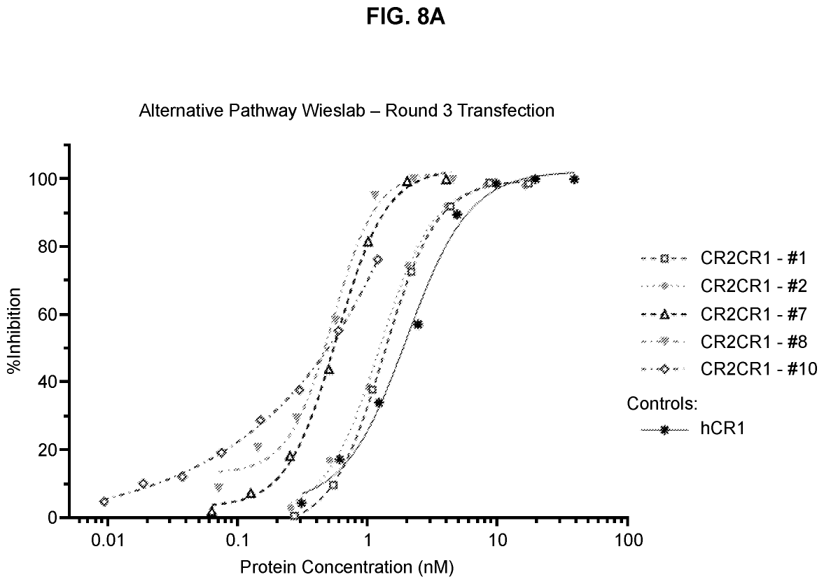

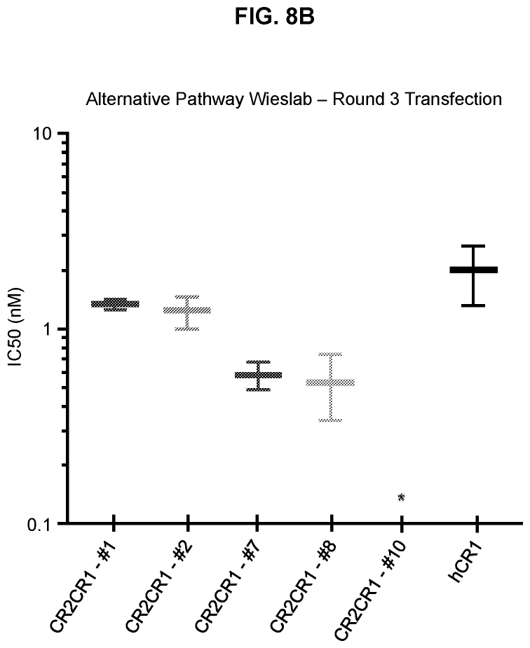

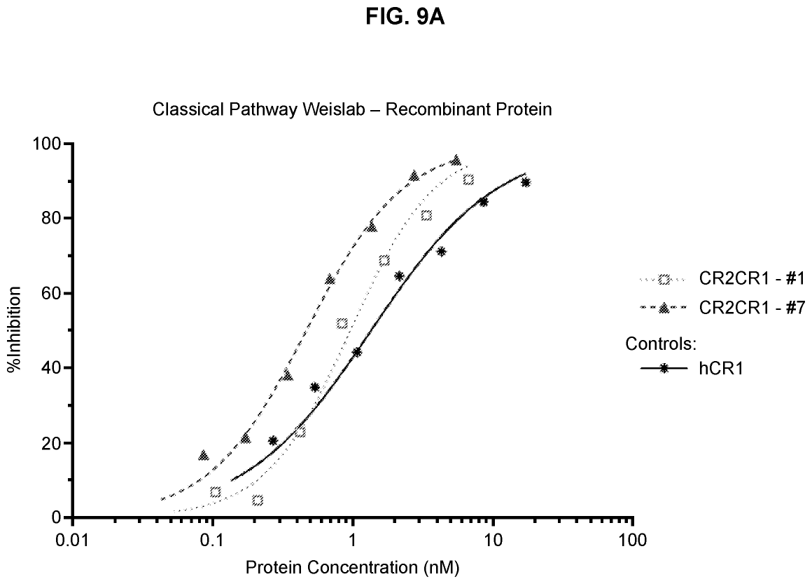

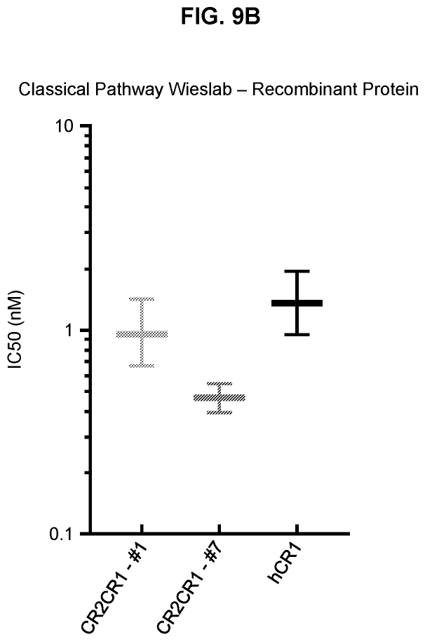

Next, classical complement pathway inhibition (FIGS. 7A-7C, FIGS. 9A-9B) and alternative complement pathway inhibition (FIGS. 8A-8B) was measured using a Wieslab® Assay.

### Classical Weislab

Transfection cell culture supernatant samples were screened for the inhibition of the human classical complement pathway using the Weislab Complement system Classical pathway ELISA (Svar). Human Recombinant CR1 (R&D Systems) served as an internal positive inhibition control and a concentrated mock transfection sample was used as a background inhibition control. To calculate sample percent inhibition, EDTA and NHS (Complement Technology, Inc) were used as the 100% inhibition and 0% inhibition values, respectively. Concentrated transfection cell culture supernatant samples and controls were serially diluted 1:2 in 40 μL of Diluent CP in a separate 96-well, non-binding dilution plate. An 80 μL volume of NHS diluted 1:70 in Diluent CP was added to each well, then 100 μL of each sample+NHS mixture was transferred to the ELISA plate. The ELISA was then performed in accordance with the manufacturer instructions and the percent inhibition and EC50 for each sample dilution series calculated.

### Alternative WeisLab

Transfection cell culture supernatant samples were screened for the inhibition of the human alternative complement pathway using the Weislab Complement system Alternative pathway ELISA (Svar). Human Recombinant CR1 (R&D Systems) served as an internal positive inhibition control and a concentrated mock transfection sample was used as a background inhibition control. To calculate sample percent inhibition, EDTA and NHS (Complement Technology, Inc) were used as the 100% inhibition and 0% inhibition values, respectively. Concentrated transfection cell culture supernatant samples and controls were serially diluted 1:2 in 40 μL of Diluent AP in a separate 96-well, non-binding dilution plate. An 80 μL volume of NHS diluted 1:70 in Diluent AP was added to each well, then 100 μL of each sample+NHS mixture was transferred to the ELISA plate. The ELISA was then performed in accordance with the manufacturer instructions and the percent inhibition and EC50 for each sample dilution series calculated.

The results of these assays are also shown in Table 3 below.

TABLE 3
CR2-CR1 Fusion Construct Inhibition Assay

| | | | Classical | Alternative |
|---|---|---|---|---|
| CR2-CR1 | | | Pathway IC50 | Pathway IC50 |
| Fusion | HEK293 Protein | | (change | (change |
| Protein | Expression | | v. CR1) | v. CR1) |
| CR2CR1 #1 | 1056-4101 | ng/mL | 1.1-3.9 nM | 1.3 nM |
| | | | (0.55-2.77) | (0.67X) |
| CR2CR1 #2 | 1373-5986 | ng/mL | 1.8-6.7 nM | 1.2 nM |
| | | | (0.90-3.54X) | (0.62X) |
| CR2CR1 #5 | 1659 | ng/mL | 2.9 nM | — |
| | | | (2.29 X) | |
| CR2CR1 #6 | 935 | ng/mL | 3.1 nM | — |
| | | | (2.48X) | |
| CR2CR1 #7 | 571-4912 | ng/mL | 0.7-1.0 nM | 0.6 nM |
| | | | (0.33-0.81X) | (0.29X) |
| CR2CR1 #8 | 744-866 | ng/mL | 0.4-0.7 nM | 0.5 nM |
| | | | (0.18-0.56X) | (0.27X) |
| CR2CR1 #9 | 833-1807 | ng/mL | 0.6-1.9 nM | — |
| | | | (0.31-0.92X) | |
| CR2CR1 #10 | 333-720 | ng/mL | 0.7-3.4 nM | [Curve |
| | | | (0.38-1.66X) | incomplete] |

Design round 2 constructs demonstrated better complement pathway inhibition activity than design round 1 constructs. Having two site 2 binding sites like native hCR1 may improve activity. Design round 2 constructs had lower protein expression than design round 1 constructs. Proteins with (GGGGS)2 linkers between CR2 & CR1 regions showed an unexpected high MW band in Western blot.

### Example 4—CR2-CR1 Fusion Construct In Vivo Expression

The mRNA and protein expression of the constructs generated in Example 1 were analyzed in C57BL6/J mice. To prepare samples for analysis, whole C57BL6/J eyeballs from the study were homogenized using a bead-based homogenization method in complete lysis buffer (MSD lysis buffer plus Halt protease inhibitor cocktail and Halt EDTA). Total protein quantification was performed using the DC Protein Assay Kit II (Bio-Rad) with protein standard albumin as the standard curve to determine protein concentration for each sample. Lysates were diluted to either 0.250 mg/mL total protein or 0.500 mg/mL (optimal total protein concentration determined during Assay Development).

To measures the concentrations of CR2-CR1 (CR2CR1 #1 or CR2CR1 #7) using a sandwich immunoassay, small spot streptavidin MSD plates were coated with 25 μL of 1 μg/mL coating antibody solution (biotinylated polyclonal capture antibody specific for the CR1 portion of CR2-CR1 fusion protein) prepared in MSD Diluent 100. Plates were sealed and incubated at room temperature for ˜1 hour with shaking at approximately 750 RPM. Following coating, the plates were washed three times with approximately 150 μL per well of MSD Wash Buffer (PBS+0.05% Tween-20). Twenty-five microliters of Assay Diluent (MSD lysis buffer+2 mg/mL BSA) was added to each of well of the assay plate. Additionally, 25 μL of standards (either utilizing CR2CR1 #1 or CR2CR1 #7 recombinant protein spiked into MSD lysis buffer+2 mg/mL BSA), controls, and ocular lysate samples were added to the appropriate wells on the assay plates, in duplicate. Plates were sealed and incubated at room temperature for ˜1 hour with shaking at approximately 750 RPM. At the end of this incubation, the plates were washed three times as described above. Twenty-five microliters of 1× detection antibody (Sulfo-tagged sheep anti-human CR2 polyclonal antibody) in MSD Diluent 3 was then added to all wells of the assay plate. The plates were sealed and incubated at room temperature for ˜1 hour with shaking at approximately 750 RPM. Finally, the plates were washed again as described above, and 150 μL of MSD GOLD Read Buffer B was added to all wells on the plates. Assay plates were then read on the MESO QuickPlex SQ 120MM within 5 minutes. The immunoassay allowed for the generation of an electrochemiluminescent response that is directly proportional to the amount of analyte detected in the well. Back-calculated concentrations (BCC) of analyte are generated using a 4PL fit model with 1/y2 weighting.

### Measurement of CR2-CR1 in Serum (Human CR2 Assay)

To measures the concentrations of CR2-CR1 (CR2CR1 #1 or CR2CR1 #7) using a sandwich immunoassay, small spot streptavidin MSD plates were coated with 25 μL coating antibody solution (1:17.5 diluted biotinylated mouse capture antibody specific for CR2 portion of CR2-CR1 fusion protein) prepared in MSD Diluent 100. Plates were sealed and incubated at room temperature for ˜1 hour with shaking at approximately 750 RPM. Following coating, the plates were washed three times with approximately 150 μL per well of MSD Wash Buffer (PBS+0.05% Tween-20). Twenty-five microliters of Assay Diluent (MSD Diluent 100) was added to each of well of the assay plate. Additionally, 25 μL of standards (either utilizing CR2CR1 #1 or CR2CR1 #7 recombinant protein spiked into mouse serum), controls, and study serum samples were added to the appropriate wells on the assay plates, in duplicate. Plates were sealed and incubated at room temperature for ˜1 hour with shaking at approximately 750 RPM. At the end of this incubation, the plates were washed three times as described above. Twenty-five microliters of 1× detection antibody (Sulfo-tagged sheep anti-human CR2 polyclonal antibody) in MSD Diluent 3 was then added to all wells of the assay plate. The plates were sealed and incubated at room temperature for ˜1 hour with shaking at approximately 750 RPM. Finally, the plates were washed again as described above, and 150 μL of MSD GOLD Read Buffer B was added to all wells on the plates. Assay plates were then read on the MESO QuickPlex SQ 120MM within 5 minutes. The immunoassay allowed for the generation of an electrochemiluminescent response that is directly proportional to the amount of analyte detected in the well. Back-calculated concentrations (BCC) of analyte are generated using a 4PL fit model with 1/y2 weighting.

### Measurement of CR2-CR1 in Serum (Human CR2-CR1 Assay)

To measures the concentrations of CR2-CR1 (CR2CR1 #1 or CR2CR1 #7) using a sandwich immunoassay, small spot streptavidin MSD plates were coated with 25 μL of 1 μg/mL coating antibody solution (biotinylated polyclonal capture antibody specific for the CR1 portion of CR2-CR1 fusion protein) prepared in MSD Diluent 100. Plates were sealed and incubated at room temperature for ˜1 hour with shaking at approximately 750 RPM. Following coating, the plates were washed three times with approximately 150 μL per well of MSD Wash Buffer (PBS+0.05% Tween-20). Twenty-five microliters of Assay Diluent (MSD Diluent 100) was added to each of well of the assay plate. Additionally, 25 μL of standards (either utilizing CR2CR1 #1 or CR2CR1 #7 recombinant protein spiked into mouse serum), controls, and study serum samples were added to the appropriate wells on the assay plates, in duplicate. Plates were sealed and incubated at room temperature for ˜1 hour with shaking at approximately 750 RPM. At the end of this incubation, the plates were washed three times as described above. Twenty-five microliters of 1× detection antibody (Sulfo-tagged sheep anti-human CR2 polyclonal antibody) in MSD Diluent 3 was then added to all wells of the assay plate. The plates were sealed and incubated at room temperature for ˜1 hour with shaking at approximately 750 RPM. Finally, the plates were washed again as described above, and 150 μL of MSD GOLD Read Buffer B was added to all wells on the plates. Assay plates were then read on the MESO QuickPlex SQ 120MM within 5 minutes. The immunoassay allowed for the generation of an electrochemiluminescent response that is directly proportional to the amount of analyte detected in the well. Back-calculated concentrations (BCC) of analyte are generated using a 4PL fit model with 1/y2 weighting.

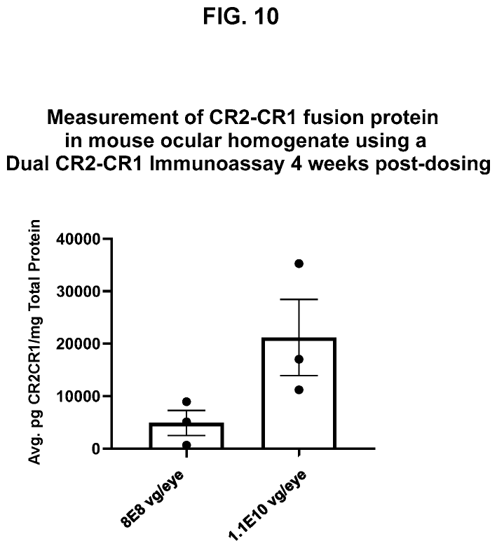

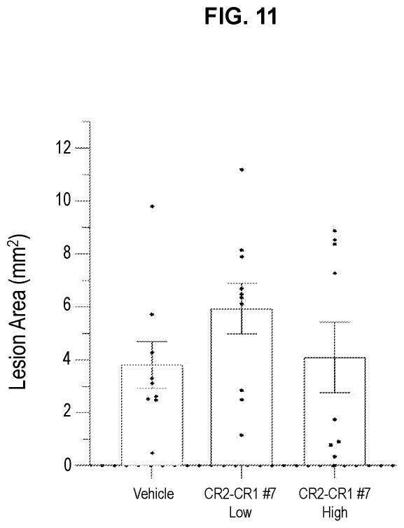

FIG. 10 shows the measurement of CR2-CR1 fusion protein in mouse ocular homogenate 4 weeks post dosing in mice dosed with either 8E8 vg/eye or 1.1E10 vg/eye of a mixed population of AAV expressing either CR2CR1-#1 or CR2CR1-#7. The data shows that the construct is successfully expressed in the eye at both concentrations tested, and that the CR2-CR1 fusion proteins can be successfully quantified.

### Example 5—Kinetics of CR2-CR1 Fusion Protein Binding to Target Ligands Using Biolayer Interferometry (BLI)

The binding kinetics of CR2-CR1 fusion proteins, human CR1, and human CR2 to their respective ligands were determined using biolayer interferometry (BLI) with a GatorPrime instrument (Gator Bio). Recombinant His-tagged human CR1 (R&D Systems), human CR2 (R&D Systems), or CR2-CR1 fusion proteins (GenScript) were immobilized onto Ni-NTA probes, and the response signals from the protein-loaded probes binding to C3d, iC3b, C3b, or C4b (Complement Tech) in a 7-point dilution series were measured for a 300 second association step, followed by a 300 second dissociation step in assay buffer. To reduce assay background/noise due to non-specific complement protein binding, a double reference was applied using a 0 nM ligand condition (Sample or Reference Probe Blank) and a series of Ni-NTA probes not loaded with protein (Reference Probe) according to the following equation: Sample Response=(Sample Signal−Reference Probe Signal)−(Sample Blank Signal−Reference Probe Blank Signal). The binding affinity constant, KD, and association/dissociation rate constants, kₒₙ/k_{off}, for all binding interactions were calculated using the GatorOne software (Gator Bio).

Table 4 shows Equilibrium dissociation constants (KD), association rates (kₒₙ), and dissociation rates (k_{off}) for complement receptor proteins binding to the CR1 ligands C3b and C4b of full length recombinant human CR1 protein (rhCR1) compared with CR2-CR1 fusion proteins.

TABLE 4
Binding Kinetics for CR1 Ligands

| | | kₒₙ | k_{off} | K_{D} | K_{D} vs. |
|---|---|---|---|---|---|
| Protein | Ligand | (1/Ms) | (1/s) | (M) | hCR1 K_{D} |
| rhCR1 | C3b | 4.42E+04 | 8.15E−04 | 1.84E−08 | — |
| CR2CR1 #1 | C3b | 6.77E+04 | 4.39E−04 | 6.49E−09 | 0.35X |
| CR2CR1 #7 | C3b | 1.15E+05 | 5.64E−04 | 4.91E−09 | 0.27X |
| rhCR1 | C4b | 4.43E+04 | 5.86E−04 | 1.32E−08 | — |
| CR2CR1 #1 | C4b | 5.60E+04 | 6.94E−04 | 1.24E−08 | 0.94X |
| CR2CR1 #7 | C4b | 6.33E+04 | 6.63E−04 | 1.05E−08 | 0.80X |

Table 5 shows equilibrium dissociation constants (KD), association rates (kₒₙ), and dissociation rates (k_{off}) for complement receptor proteins binding to the CR2 ligands iC3b and C3d of full length recombinant human CR2 protein (rhCR2) compared with CR2-CR1 fusion proteins.

TABLE 5
Binding Kinetics for CR2 Ligands

| | | kₒₙ | k_{off} | K_{D} | K_{D} vs. |
|---|---|---|---|---|---|
| Protein | Ligand | (1/Ms) | (1/s) | (M) | hCR2 K_{D} |
| rhCR2 | iC3b | 1.48E+05 | 9.65E−04 | 6.51E−09 | − |
| CR2CR1 #1 | iC3b | 1.21E+05 | 6.84E−04 | 5.66E−09 | 0.87X |
| CR2CR1 #7 | iC3b | 1.40E+05 | 5.82E−04 | 4.15E−09 | 0.64X |
| rhCR2 | C3d | 5.36E+04 | 5.59E−04 | 1.04E−08 | − |
| CR2CR1 #1 | C3d | 4.63E+04 | 4.03E−04 | 8.70E−09 | 0.84X |
| CR2CR1 #7 | C3d | 5.10E+04 | 3.99E−04 | 7.83E−09 | 0.75X |

Taken together, this data shows that the CR2-CR1 construct successfully binds to both CR2 and CR1 ligands of interest more strongly than rhCR2.

### Example 6—Treatment of Stargardt Disease in Mice Using AAV2-smCBA-CR2-CR1 (Prophetic)

To evaluate the in vivo efficacy of AAV2-smCBA-CR2-CR1, 129S1/Sv1mJ mice and Abca4 knockout mice exhibiting phenotypic Stargardt disease will have the construct subretinally administered to reduce A2E accumulation. Abca4−/− mice model of Stargardt disease is like humans, including A2E accumulation and retinal degeneration. It is useful for studying retinal disorders like Stargardt disease or geographic atrophy. 129S1 mice have ocular anatomy that in some respects is like primate/human eyes and is ideal for investigating mechanisms of retinal degeneration. Retinal pigment epithelium, photoreceptors, and supportive matrices are present. Study design and treatment groups are shown in Tables 6 and 7.

TABLE 6
Study Design

| N | Species | Strain/Type | Sex | Age | Weight | Notes |
|---|---|---|---|---|---|---|
| 10 | Mouse | 129S1 | m/f | 4-wks | TBD | 5 m, 5 f per group |
| 40 | Mouse | ABCA4^{-/-} | m/f | 4-wks | TBD | 5 m, 5 f per group |

TABLE 7
Treatment Groups

| Study | | | | | Dose | | |
|---|---|---|---|---|---|---|---|
| Group | Test Article | Route | Dose vol. | Eye | (vg/eye) | N | Necropsy Day |
| 1 | 129S1 + Vehicle | n/a | — | — | n/a | 10 | 91 |
| 2 | ABCA4^{-/-} + Vehicle | Subretinal | 1 μL | OU | n/a | 10 | 91 |
| 3 | AAV2.CR2CR1 #1 | Subretinal | 1 μL | OU | 8E8 | 10 | 91 |
| 4 | AAV2.CR2CR1 #7 | Subretinal | 1 μL | OU | 8E8 | 10 | 91 |
| 5 | AAV2.CR2CR1 #7 | Subretinal | 1 μL | OU | 3E9 | 10 | 91 |

Selected doses of AAV2.anti-C3 vectors will be used for characterizing their pharmacology and biodistribution following subretinal injection, based on previous studies using GA mouse models. These data will inform the long-term efficacy (>3 months) of the vectors in mitigating the Stargardt and geographic atrophy phenotypes. Healthy control and Abca4−/− groups will be included, with a single subretinal vehicle control providing no and high efficacy data points. Dosing of 4-week-old Abca4−/− and WT mice will be conducted.

AAV2.anti-C3 vectors will be supplied frozen in concentrated form. The test article will be thawed at room temperature on the day of use and formulated to the desired dose. Animals will be observed as described in Table 8. Mouse eyes will be dilated and the animals will be anesthetized according to standard operating procedures. The mice be placed on a regulated heating pad, and the posterior pole will be visualized under magnification. A 12.7 mm 30-gauge insulin syringe will be used to puncture the cornea just above the corneal limbus, avoiding any contact with the sclera and lens. The transvitreal subretinal injections will be performed using a 10 μL Hamilton syringe with a 33-gauge blunt needle inserted through the corneal puncture across the vitreous, with the shaft aimed at the back of the eyecup, avoiding any trauma to the lens or iris. A total volume of 1 μL will be delivered.

TABLE 8
In vivo assessments and monitoring

| Observation | Timepoint(s) | Notes |
|---|---|---|
| Standard clinical | 2x daily, with attention | All groups |
| | to eyes | |
| Bodyweight | Day 0 (pre-treatment), 1, | All groups |
| | 14, 28, 42, 56, 70, 84, 91 | |
| Food consumption | Daily | All groups; visual assessment |
| | | only |
| Dosing observations | Day 0 (pre-treatment), | All groups. |
| | Day 0 (1-hr post-treatment) | |

Detailed observations will be made once
every two weeks and on the day of necropsy.

| | | |
|---|---|---|
| Ophthalmic | 2x daily, with attention | Clinical observations are as |
| examinations | to eyes | described above. |
| Blue-light | Day 0 (Prior to dosing), | 488 and 518 nm modes. |
| Autofluorescence | 42, 91 | |
| Optical Coherence | Day 0 (Prior to dosing), | Spectral domain optical coherence |
| Tomography | 42, 70, 91 | tomography (OCT) will be conducted |
| | | on both eyes per animal. |
| Whole blood | Day 91 | At necropsy, terminal bleed to |
| | | collect ≥500 μL blood and |
| | | processing of said blood to serum. |
| Retinal | Day 91 | At necropsy, 10 eyes per group (1 |
| Chromatography | | eye from each animal) will be |
| [A2E LC-MS/MS] | | processed per EyeCRO protocol for |
| | | LC-MS/MS quantification of A2E. |
| Vector DNA and | Day 91 | Tissues designated for vector DNA |
| mRNA analysis | | analysis will be proceed in RNAlater |
| | | and flash frozen. 1 eye from each |
| | | animal will be observed (5 eyes per |
| | | group). |
| Protein analysis | Day 91 | Tissues designated for protein |
| | | analysis will be rinsed with cold |
| | | buffer or PBS to remove excess blood. |
| | | The tissue will then be weighed, |
| | | flash frozen, and processed for |
| | | protein analysis only if needed (5 |
| | | eyes per group). Diluted humor and |
| | | whole globe will be flash frozen |
| | | and processed for protein analysis |
| | | only if needed. |
| Terminal | Day 91 | Organ weights and tissue retention |
| Observations | | for all animals of the eye and brain. |
| | | Tissues in accordance with regulatory |
| | | requirements (to include reporting |
| | | as tissue: bodyweight and tissue: |
| | | brain weights) for ocular AAV studies. |

### Body Weights

The body weights of individual animals will be measured and recorded at the specified timepoints.

### Blue Autofluorescence (BAF) Imaging

Animals will be sedated with ketamine/xylazine and eyes will be dilated according to standard procedures. Images will be acquired using a Spectralis® HRA+OCT laser ophthalmoscope (Heidelberg Engineering, Inc.) using the high-resolution setting at a plane of highest reflectance determined by infrared imaging to indicate the retinal pigment epithelium.

### Optical Coherence Tomography

Animals are anesthetized and eyes are dilated according to standard procedures. Following sedation and dilation, animals are secured on a platform and presented to a Micron IV OCT module (Phoenix Research Inc.) for imaging. The total retina thickness will be measured from vertical scans and horizontal scans acquired within each eye, and the data is plotted as the average retinal thickness across the scans. Total retinal thickness, and outer nuclear layer thickness, will be independently measured.

### Serum Collection

Animals will be sedated by IP administration of Ketamine/Xylazine, and puncture will be performed to isolate whole blood (˜500 μL) into yellow capped SST tubes. The tube will sit at RT for 30 minutes, and then be centrifuged for 3 minutes at 10,000×g. The total serum volume (˜50% of whole blood volume) will be transferred to a snap-cap tube and stored at −80° C. until shipment.

### Collection of Eyecups for A2E Analysis

Animals (n=10 mice/arm) will be sedated by IP administration of Ketamine/Xylazine and then euthanized by intracardiac administration of Euthasol®. Following euthanasia, the left eyes will be dissected to isolate the PECRS complex containing RPE/choroid/retina/sclera (eyecups), which will be individually snap-frozen in liquid N2, placed in a snap-cap tube, and stored at −80° C. The tissue will be individually processed for A2E quantification at EyeCRO.

### Collection of Ocular Tissues for Storage in RNAlater

Following euthanasia, the eyes will be enucleated and placed in purple screw-cap tubes with ≤500 μL RNAlater at 4° C. for 24 hours, and then stored at −80° C. until shipment to the client on dry ice.

### Collection of Ocular Tissues for Future Protein Analysis

Following euthanasia, the eyes will be enucleated and collected from each eye in pre-weighed tubes and immediately snap frozen in liquid N2 and stored at −80° C. Tissue weight ((tube+tissue weight)−tube weight=tissue weight) will be measured and recorded.

### A2E Quantification

A2E will be extracted using a modified Folch method to extract all ocular lipids. Frozen eyecups are homogenized using steel beads in a solution of DPBS, methanol, and chloroform. This homogenate is then transferred to a clean tube and the original tube is washed once with the same solvent mixture. The homogenate plus wash is centrifuged, and the organic layer is transferred to a new tube. The aqueous layer is then washed with pure chloroform once, and the chloroform layer is combined with the first organic layer. The organic layer is then dried under vacuum and reconstituted in acetyl nitrile for HPLC-MS/MS analysis.

### Example 7—Pharmacodynamic Evaluation of AAV2.CR2CR1 Constructs with Subretinal Administration in a Sodium Iodate Mouse Model of Geographic Atrophy

This study evaluated the efficacy of AAV2-smCBA-CR2-CR1 in a C57BL/6 mice sodium iodate (NaIO₃) induced retinal injury model. Sodium iodate treatment induced damage to retinal pigment epithelium (RPE) cells, which are needed for preserving photoreceptor cells and can simulate the pathology of geographic atrophy. Previous research established that a dose of 25 mg/kg NaIO₃ elicits minimal escape responses while causing lasting damage to RPE cells. Sodium iodate was administered via intraperitoneal injection (IP), as this administration route usually produces the desired retinal degeneration profile for the evaluation of the test article and is phenotypically akin to the retinal pathology seen in patients with Geographic Atrophy.

The AAV2.CR2CR1 constructs were administered through subretinal injection, which targets the same anatomical structures in a comparable manner to suprachoroidal (SCS) but without the constraint of requiring a specific device for access in the animal model. The vehicle was administered via subretinal injection to mirror the administration route employed for the test therapeutic constructs.

The study design is disclosed in Table 9.

TABLE 9
Sodium Iodate Mouse Model Study Design

| | | | | | Sodium | | |
|---|---|---|---|---|---|---|---|
| | N | | | Volume/ | Iodate | In-Life | Time of Euthanasia and |
| Grp# | Animals | Treatment | Dose | ROA | Induction | Assessments | Tissue Collection (N Eyes) |
| 1 | 6 | No | NA | NA | NA | OE: Baseline | Day 39: |
| | | Treatment | | | | for enrollment | All animals: Blood |
| | | | | | | and Days 25 | collection (for serum) and |
| 2 | 10 | Vehicle | 0 | 1 μL | 25 mg/kg | and 39 | collection of eye and brain |
| | | | | Subretinal | IP | OCT: Baseline, | weights |
| | | | | Oculus | Day 29 | and immediately | N = 10/grp (N = 6 from Grp |
| | | | | Uterque | | post dose (for | 1) OS for RPE/choroid |
| | | | | Day 1 | | SR confirmation) | flatmounts |
| | | | | | | and Days 8, | N = 10/grp (N = 6 from Grp |
| | | | | | | 25, and 39 | 1) OD for VH collection, |
| | | | | | | ERG: Baseline | then separated as follows: |
| | | | | | | and Days 25, | N = 5 (N = 3 from Grp 1) OD |
| 4 | 10 | AAV2.CR2CR1 | 8E8 | | | and 39 | for vector DNA/mRNA |
| | | #7 | GC/eye | | | | analysis processed in |
| | | | | | | | RNAlater and snap frozen |
| 5 | 10 | AAV2.CR2CR1 | 3E9 | | | | N = 5 (N = 3 from Grp 1) OD |
| | | #7 | GC/eye | | | | for protein analysis and |
| | | | | | | | snap frozen |

### Intraperitoneal (IP) Dosing Procedures—Sodium Iodate Model Induction (Groups 2-5)

Sodium iodate (NaIO₃) was dissolved in 0.9% sterile saline just prior to use. Briefly, mice were manually restrained and a 25 G needle was used to administer NaIO₃ intraperitoneally (IP) at 25 mg/kg in a dosing volume of 10 mL/kg.

### Subretinal (SR) Dosing Procedures

Sedation and preparation. On the days indicated, animals received buprenorphine (0.01-0.05 mg/kg SC) for pain management. A cocktail of 1% tropicamide HCl and 2.5% phenylephrine HCl was applied topically to dilate and proptose the eyes, if not already dilated. If not already sedated, animals were sedated with a ketamine/xylazine cocktail (80-90/10-20 mg/kg IP). Following anesthetic induction, one drop of 0.5% proparacaine HCl was applied topically.

Procedure. The procedure was aseptically performed by a veterinary ophthalmologist or trained technician. The cornea was kept moistened using topical eyewash (BSS), and body temperature was maintained using a heated surgical table and hot pads as needed. A small pilot hole using the tip of a 30-gauge needle was made for subretinal injection of 1 μL test article or vehicle using a 34-gauge needle and a 10 μL syringe. After dispensing the syringe contents, the syringe needle was slowly withdrawn. This procedure was repeated for the contralateral eye. Subretinal injection volumes are specified in the experimental design table. Following injections, animals underwent optical coherence tomography (OCT) imaging to confirm dose placement. If at any time during the surgical procedure following OCT scanning, the surgeon determined the injection was suboptimal, or not successful, the animal was euthanized and replaced. Following the injection procedure, 1 drop of 0.3% ofloxacin ophthalmic solution or neomycin polymyxin B sulfates gramicidin ophthalmic solution and eye lubricant was applied topically to the ocular surface. Animals received atipamezole (0.1-1.0 mg/kg, IM) to reverse the effects of the anesthetic.

Post-Operative Care. Animals returned to their cages and were allowed to recover normally. No special post-operative care was necessary aside from assuring the animals resume normal activity.

### Ocular Examinations

A veterinary ophthalmologist or trained scientist performed complete ocular examinations using a slit lamp biomicroscope and indirect ophthalmoscope and lens to evaluate anterior and posterior segment clinical observations for all animals at the timepoints indicated. A topical mydriatic (1 drop of Tropi-Phen: 1% tropicamide and 2.5% phenylephrine HCl per eye) was given following the anterior segment examination to facilitate examination of the posterior segment. All animals that had normal baseline ocular examinations were considered for enrollment. Animals were not tranquilized for the examinations.

### Full Field Electroretinography (ERG)

Sedation. On the days indicated, if not already sedated, animals were sedated with a ketamine/xylazine cocktail (80-90/10-20 mg/kg IP).

Procedure. Animals were dark adapted for at least twelve (12) hours prior to ERG. ERGs were performed on both eyes using the Diagnosys ERG systems. Under dark adaptation, a cocktail of 1% tropicamide HCL and 2.5% phenylephrine hydrochloride was applied topically to dilate the eyes, if not already dilated. Before ERGs were recorded, pupil dilatation was checked to ensure full dilation. Animals were first positioned on the ERG machine, then 0.5% proparacaine and eye lubricant was applied to the eyes, followed by the electrode contact and reference leads. Animals were placed on a warm water blanket to control body temperature and contact lens leads was placed on the eyes. A reference subcutaneous lead was placed in the head and a ground lead placed near the tail of the animal. Light adapted ERG with 30 cd·m-2 background light immediately followed the dark-adapted series. There was a 5 min light-adaptation period before the light-adapted signals are collected. Background light was used constantly for all light-adapted measurements.

ERG parameters were as follows:

### 1) Dark-Adapted ERG:

Step 1-9: 0.001-10 cd·s·m-2 scotopic ERG, each step advancing half log units in light intensity. Both ERG traces and oscillatory potentials were collected (where applicable: a-wave, b-wave, c-wave).
Step 10: Dark-adapted 150 cd·m-2 scotopic traces and oscillatory potentials were collected (where applicable: a-wave, b-wave, c-wave).

### 2) Light-Adapted ERG:

Step 1-3: 1-10 cd·s·m-2 photopic ERG, each step advancing half log units in light intensity. Both ERG traces and oscillatory potentials were collected (where applicable: a-wave, b-wave, c-wave).
Step 4-7: 3.0 cd·s·m-2 photopic ERG flicker at 10-40 Hz, each step advancing 10 Hz. Both ERG traces and oscillatory potentials were collected (where applicable: a-wave, b-wave, c-wave).

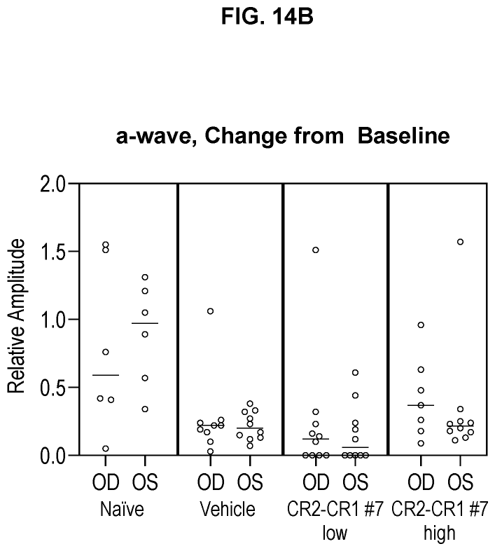

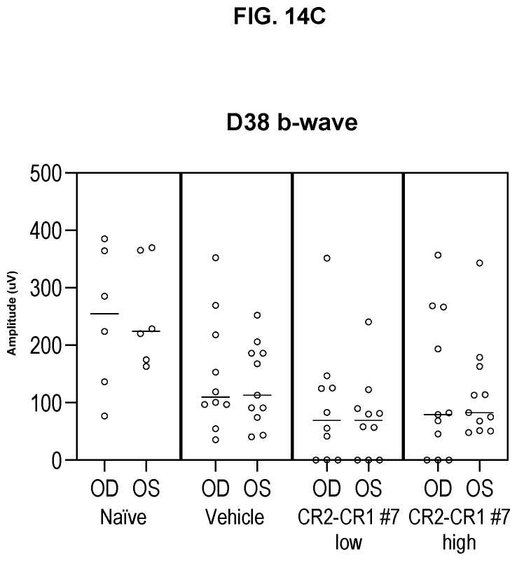

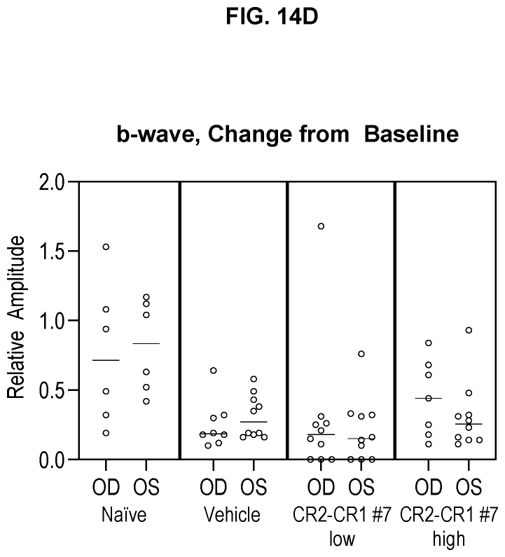

FIGS. 14A-14D shows the a-wave (FIG. 14A), a-wave change from baseline (FIG. 14B), b-wave (FIG. 14C), and b-wave change from baseline (FIG. 14D) at day 38.

### Optical Coherence Tomography (OCT)

Sedation. Animals were sedated for the procedure with a ketamine/xylazine cocktail (80-90/10-20 mg/kg IP), and one drop of 0.5% proparacaine HCl was applied to both eyes.

Procedure. At timepoints indicated, animals underwent OCT imaging procedures of the posterior section of the eye to determine retinal changes over time using the Heidelberg Spectralis OCT. Eyes were dilated using a cocktail of tropicamide HCL 1% and phenylephrine hydrochloride 2.5% for OCT 15 minutes prior to examination. A series of b-scans was collected to capture the entire posterior segment with the optic nerve head oriented centrally such that half of the b-scans were superior and half inferior to the optic nerve. Following the procedure, animals received atipamezole (0.1-1.0 mg/kg IM) to reverse the effects of the anesthetic.

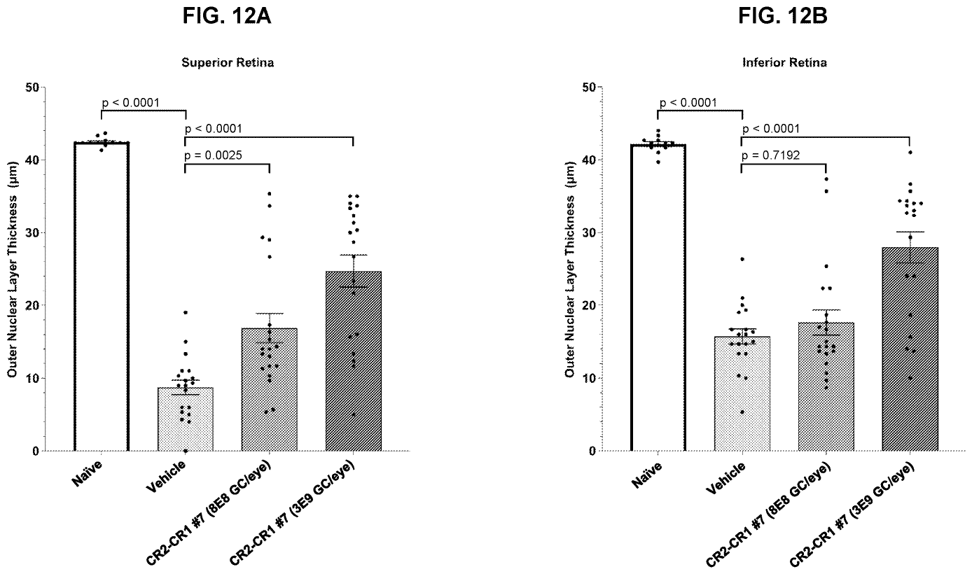

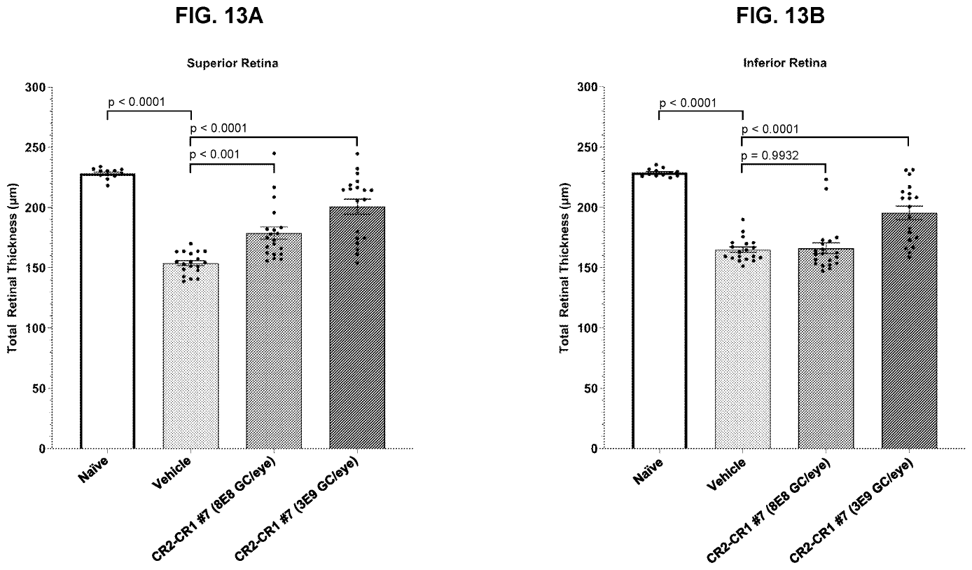

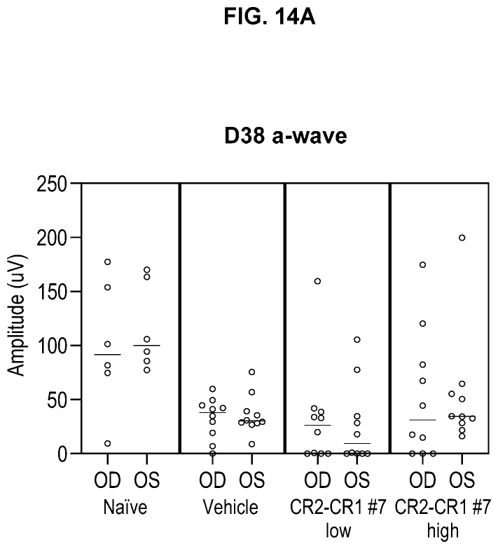

Data Analytics. Outer nuclear layer (ONL) and total retinal thickness (TRT) were measured at three positions (left, right, and center) from a superior OCT scan. ONL thickness was measured at the superior retina (FIG. 12A) and inferior retina (FIG. B). Total retinal layer thickness was measured at the superior retina (FIG. 13A) and inferior retina (FIG. 13B).

### Euthanasia Tissue Collections

At the timepoints indicated, animals were euthanized via carbon dioxide asphyxiation and death was confirmed by cervical dislocation or thoracotomy. Following euthanasia, all eyes were enucleated, weighed, and processed. The brain of each animal was collected and weighed.

### Terminal Blood Collection

Immediately following euthanasia with CO2, a 25 G needle was inserted into the heart and the animal was exsanguinated and cervical dislocation or thoracotomy was used to confirm death. Blood was collected into a 1.5 mL RNase/DNase-free microfuge tube and time of collection was noted on the paperwork. Following collection, blood was allowed to clot at room temperature for 15-20 minutes prior to serum processing. Samples were centrifuged at room temperature for 10 minutes at 2,500 rpm in a benchtop microfuge. The serum was then transferred to a new polypropylene tube, snap frozen in liquid nitrogen, and was held at approximately −80° C.

### Eyes Designated for RPE Choroid Flatmounts

Eyes allocated for flatmount analysis were enucleated, weighed, and immediately fixed in 4% paraformaldehyde in phosphate-buffered saline (PBS) and stored overnight at approximately 4° C. The following day the eyes were transferred to cold Immunocytochemistry (ICC) buffer (PBS containing 0.5% BSA and 0.2% Tween 20) until processing.

Using a dissecting microscope, the eye was trimmed of extraneous tissue and the anterior segment and lens were removed. The retina was detached and removed from the optic nerve head with fine curved scissors. If needed, precision tools such as an eyelash knife were used to remove scarred retina from the RPE/Choroid. Resulting eye cups were rinsed with cold ICC buffer. Eye cups were incubated overnight at 4° C. in 1/100 rabbit anti-Ki67 (Cell Signaling Technologies cat #9129S). The following morning, eye cups were washed extensively in ICC buffer and were incubated for 1 hour at 4° C. in 1/100 donkey anti-rabbit biotinylated secondary antibody (Jackson Immuno cat #711-065-152). Eye cups were washed and incubated with the following antibodies at 4° C. with gentle rotation for 2-3 hours: 1/200 streptavidin-Cy5, 1/1,000 DAPI (nuclear stain; ThermoFisher cat #D1306), 1/500 Alexa Fluor™ Plus 555 Phalloidin (actin; ThermoFisher cat #A30106) and 1/100 mouse ZO1 AlexaFluor 488 (apical junctions; ThermoFisher cat #339188). Eye cups were washed extensively with cold ICC buffer. Radial cuts were made toward the optic nerve head, and the sclera-choroid/RPE complexes will be flat mounted, covered and sealed. Ten (10×) magnification was used to generate a tiled image of the entire flatmount on an Olympus Bx63 upright microscope using Olympus CellSens software.

### Flatmount Lesion Area Analysis

Following image acquisition for all flatmounts, the area of iodate damage (i.e. lesion area) were measured in Fiji/ImageJ using the polygon selection tool. The lesion area was defined as the area encompassing the scar (absence of ZO1/phalloidin+ cells) but not hypertrophic cells expression ZO1. Measurements were provided in mm² per eye and per group (See FIG. 11). In the event that multiple lesions were noted within a given eye, all lesions were measured, and the total sum of the lesion area was presented for that eye. No significant differences between vehicle and CR2-CR1 #7 were seen.

### Eyes Designated for Whole Globe Collection

Eyes were collected for biodistribution (vector DNA/mRNA) and CR2-CR1 (protein) analysis.

Vitreous Humor was collected from 10 OD eyes/group. Standard glass pipettes were purchased from World Precision Instruments (100 mm length, 100 m outer diameter) for vitreous humor collection. Using a stage micrometer and a microscope, micropipette tips were broken (100 m outer diameter) at an angle to facilitate penetration into the eye. The pipette tip was inserted at a 450 angle into the vitreous cavity, 2 mm posterior to the limbus. Vitreous humor was aspirated and, within each group, pooled into a single pre-weighed 500 μL nuclease-free polypropylene tube and then re-weighed to determine volume of vitreous added. To each tube of pooled vitreous, 10 μL of phosphate-buffered saline containing protease inhibitor (Roche Complete Mini; dilute 1 tablet in 10 mL PBS) was added. Samples were centrifuged at 6,000 rpm for 30 seconds, snap frozen in liquid nitrogen, and stored at approximately −80° C.

Following vitreous humor collection, the eyes were enucleated and weighed for eyes designated for vector DNA/mRNA analysis and for protein analysis.

Eyes designated for vector DNA/mRNA analysis (N=5 eyes/group) were placed into a screw top nuclease-free polypropylene tube pre-weighed with ˜1.5 mL RNALater and re-weighed. The tissue was kept in RNAlater and stored at approximately 4° C. for minimal 18 hours before removing RNALater and transfer to storage at approximately −80° C.

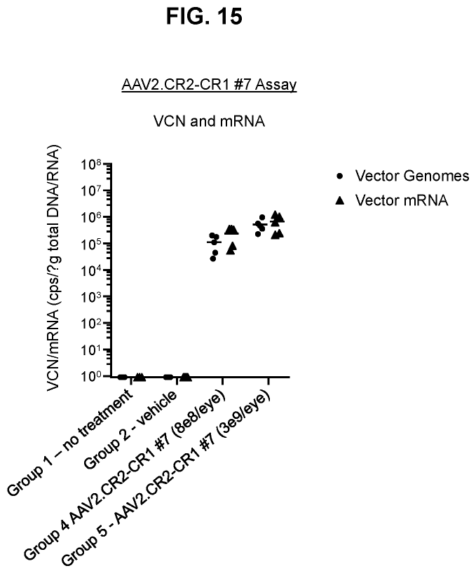

Vector DNA/mRNA was measured for CR2-CR1 #7 (FIG. 15). Dose dependent increased in vector genomes and mRNA levels for CR2-CR1 #7 was observed.

Eyes designated for protein analysis (N=5 eyes/group) were placed into a pre-weighed screw top nuclease-free polypropylene tube, re-weighed, and then snap frozen in liquid nitrogen and stored at approximately −80° C.

Pre-weighed whole ocular globes from C57BL6/J mice were transferred into chilled Precellys tubes containing 6 zirconium oxide Precellys beads. Complete Lysis Buffer (MSD lysis buffer, 100× Halt Protease Inhibitor Cocktail, 100× Halt EDTA) was added to each tube using the following formula (Weight in mg×20 μL). Samples were homogenized using the Precellys homogenizer using the tumor lysis program (6500 RPM 30 Sec, 3×). Following homogenization, samples were centrifuged and evaluated for total protein yield using the Bio-Rad DC quantification assay.

Ocular homogenate protein levels were analyzed for CR2-CR1 #7.

Ocular lysate samples were diluted 60 μg total protein in TBS buffer and 4× Laemmli loading buffer. Positive controls (C3, C3b, and iC3b protein fragments) were diluted to 10 ng total protein in TBS buffer and 4× Laemmli loading buffer. Twenty microliters of diluted samples and positive controls were loaded along with 7 μL of Pageruler Plus Pre-Stained Protein ladder on a 12% Tris Glycine Gel. The gel was run at 225V constant voltage for approximately 45 minutes.

Briefly, the gel was separated from its cassette and rinsed in diH2O. Protein bands were transferred to a PVDF membrane using the iBlot2 transfer device. Following transfer, the membrane was blocked for at least 30 minutes in Pierce Protein-Free Blocking Buffer, TBS at room temperature with gentle rocking. During blocking, the primary antibody, Rabbit anti-murine-C3/C3b (EPR19394 from Abcam) was diluted 1:1000 in Pierce Protein-Free Blocking Buffer, TBS. After the blocking step, blocking buffer was removed, primary antibody solution was added to the blot, and it was placed at 4° C. overnight with gentle rocking. The next day the blot was rinsed three times with wash buffer (1×TBS+0.1% Tween 20) allowing the blot to sit in the wash buffer for 5 minutes between washes. During the wash step, the secondary antibody, Goat anti-rabbit HRP (PI31460 from Invitrogen) was diluted 1:5000 in Pierce Protein-Free Blocking Buffer, TBS. Next, the blot was placed in the secondary antibody solution and incubated at room temperature for approximately 1 hour with gentle rocking. After secondary antibody incubation, the blot was washed as described above. During the wash step, the chemiluminescent substrate (ECL Western Blotting Solution from BioRad) was prepared by adding 1 mL of Substrate 1 to 1 mL of Substrate 2. The prepared substrate was added to the surface of the blot and incubated for approximately two minutes wrapped in saran wrap. Finally, the blot was imaged on the iBright gel imager using the chemiluminescent setting.

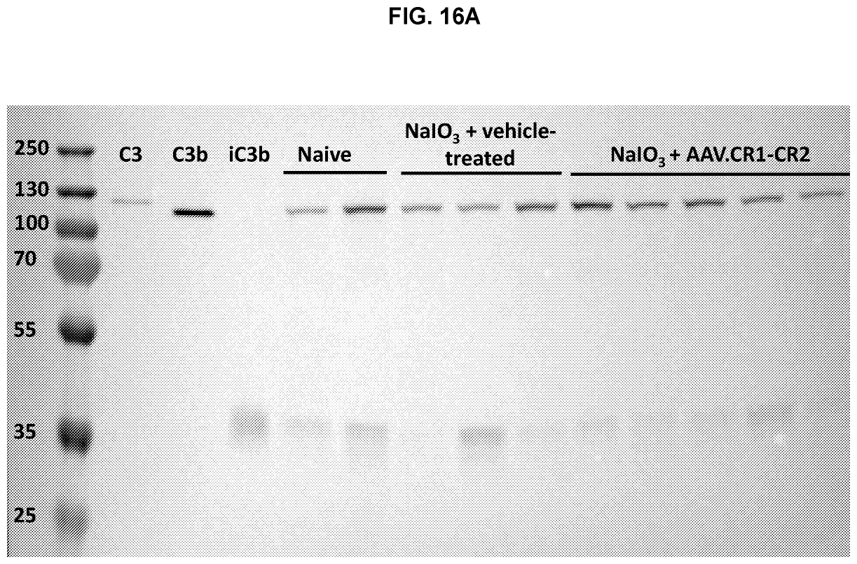

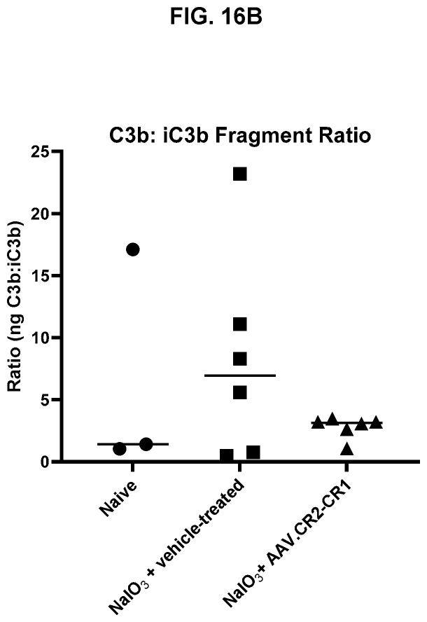

Gel images (g2i files) were exported and analyzed using Invitrogen iBright Analysis software. The bands from positive controls and ocular lysate samples were first compared against the ladder to determine their molecular weight. Bands in ocular lysate samples were determined to be either C3b or iC3b by molecular weight analysis (FIG. 16A). Since the positive controls were pure recombinant protein with a single band of known concentration (10 ng), an absolute concentration quantification was performed. The density of pixel deposit as well as the volume (area of the band on the gel) for each positive control was compared against the respective band of the same molecular weight in the study samples. Thus, an absolute concentration for C3b and iC3b bands in each study sample was calculated by the software. These concentrations were then ratioed in Excel to obtain a ratio of C3b: iC3b for each ocular lysate sample (FIG. 16B).

### Summary

This study evaluated the effect of AAV2.CR2-CR1 #7 for preserving photoreceptor outer nuclear layer thickness and retinal function in a model of NaIO₃-induced atrophy of the RPE. Retinal flatmounts showed successful induction of retinal lesions in the animals treated with NaIO₃. OCT analysis revealed decreased ONL and TRT thickness in vehicle-treated eyes compared to naïve animals. AAV2.CR2-CR1 #7 treatment at doses of 8E8 vg/eye and 3E9 vg/eye preserved ONL and TRT thickness compared to vehicle-treated mice in the superior retina (FIG. 12A), but only showed preserved ONL and TRT at the high-dose 3E9 vg/eye group in the inferior retina. ff-ERG showed no significant differences in a-wave and b-wave amplitudes between the treatment groups. Analysis of ocular homogenates showed a dose-dependent increase in vector and mRNA levels for CR2-CR1 #7 (FIG. 15). Due to the significant preservation of retina thickness across the entire retina, 3E9 vg/eye was considered an efficacious dose in this model.

AAV2.CR2-CR1 #7 treatment resulted in a decrease in the ratio of C3b:iC3b complement fragments in ocular homogenate from the treated animals by reducing the concentration of C3b and increasing the concentration of iC3b. In this assay, the activation product C3b was understood to have become covalently attached to activating surfaces and the initial building block for the generation of additional C3 and C5 convertase units. Inhibition of the C3 convertase would reduce the amount of C3b deposited on the cell. In addition, surface-bound C3b is degraded to iC3b, C3d, and C3dg with the aid of CR1, and is primary target of opsonins that recruit phagocytic cells. Thus, NaIO₃ treatment increased the activation of the complement pathway and the generation of C3b. The results show that AAV.CR2-CR1 #7 reduced the ratio of C3b:iC3b proteins in the treated animals.

### Example 8—Evaluation of AAV2.CR2-CR1 #7 Toxicity, Immunogenicity, and Biodistribution after Suprachoroidal Space Injection in Non-Human Primates

To evaluate the toxicity, immunogenicity, and biodistribution of AAV.CR2-CR1 #7, cynomolgus monkeys received a single bilateral suprachoroidal space (SCS) injection and were observed over the course of a 12-week study (Table 10).

TABLE 10
Non-Human Primate Study Design

| | | | | Dose | Dose | | |
|---|---|---|---|---|---|---|---|
| | | | Day of | Level | Concentration | No. of Animals | |
| Group | Left Eye | Right Eye | Dosing | (vg/eye) | (vg/mL) | Males | Females |
| 1 (Control) | Vehicle | Vehicle | 1 | 0 | 0 | 1 | 1 |
| 2 (Low) | AAV2.CR2- | AAV2.CR2- | 1 | 3E11 | 2.5E12 | 2 | 1 |
| | CR1 #7 | CR1 #7 | | | | | |
| 3 (Mid) | AAV2.CR2- | AAV2.CR2- | 1 | 1E12 | 8.3E12 | 2 | 3 |
| | CR1 #7 | CR1 #7 | | | | | |
| 4 (High) | AAV2.CR2- | AAV2.CR2- | 1 | 3E12 | 2.5E13 | 3 | 2 |
| | CR1 #7 | CR1 #7 | | | | | |

One animal/sex/group in Groups 3 and 4 were sacrificed for interim analysis on Day 29. All remaining animals in Groups 1 through 4 were designated for terminal sacrifice on Day 85.

Dose levels were selected based on preliminary efficacy data at 8E8 and 3E9 vg/eye in rodent models scaled to non-human primates (NHPs), as well as the maximum feasible dose due to volume and titer limitations. Animals were confirmed negative for AAV2 mAbs using a cell-based assay before dosing and were immunosuppressed with intramuscular (IM) methylprednisolone 10 mg/kg weekly from Day −1 to 21 and 5 mg/kg weekly from Day 28 to 35. 1/sex in the mid- and high-dose groups were sacrificed on Day 29, and the remaining 1-2/sex/group were sacrificed on Day 85 (Week 12).

### Confirmation of Suprachoroidal Space Injection

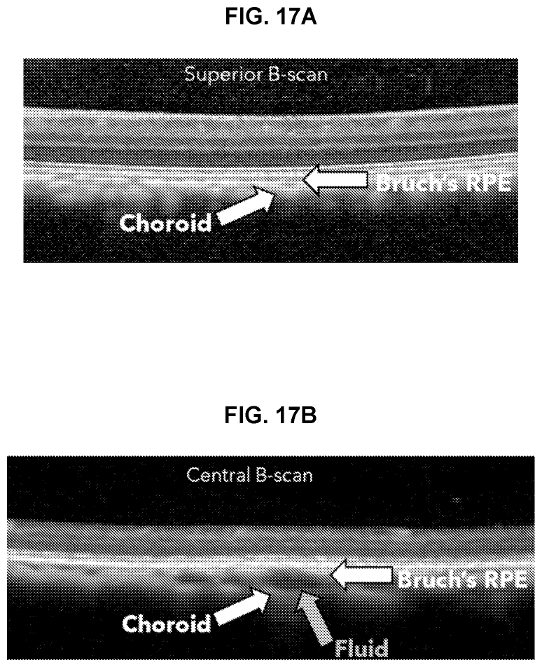

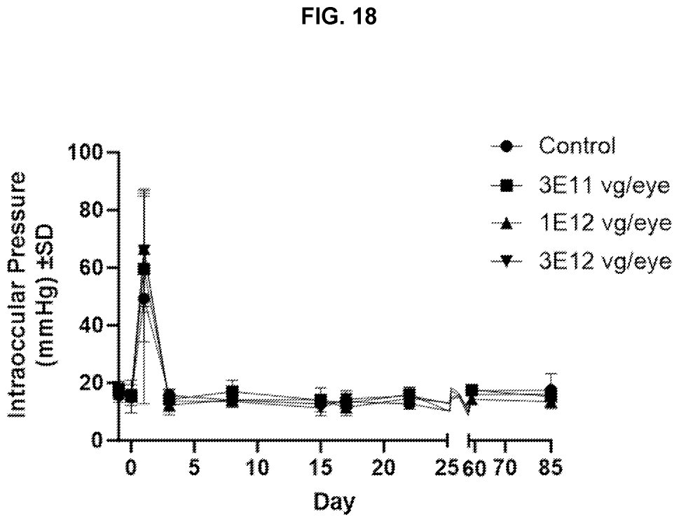

Immediately post-dosing, fluid pockets were visible in optical coherence tomography (OCT) central B-scans between the choroid and Bruch's-retinal pigment epithelium (RPE) complex, consistent with the location of the SCS (FIGS. 17A-17B). Transient elevation in intraocular pressure (IOP), which returned to baseline by the subsequent measurement and did not change again throughout the course of the study, was noted across all groups, consistent with SCS delivery (FIG. 18) (Chiang 2017). Based on OCT and IOP changes, SCS delivery was considered successful for all study animals.

### Ophthalmic Examination

In-life observations indicated the SCS administration and CR2-CR1 #7 was generally well tolerated across all doses. There were no test article-related effects on clinical observations, body weight, food consumption, tonometry (FIGS. 17A-17B), fluorescein angiography (FA), full-field electroretinography (ff-ERG), or clinical pathology (data not shown) at any timepoint examined. Ophthalmoscopic examination revealed perilimbal corneal scar at the site of aqueous humor sampling in all animals. Other than scarring, no other findings were noted in vehicle control animals. In 3 of 26 eyes administered ≥1E12 vg/eye CR2-CR1 #7, there was a mild increase in anterior and posterior segment intraocular inflammation (Grade 1 to 2) on Day 22. These events were sporadic, not observed in the contralateral eye, and reduced or resolved in severity by the next observation, as by D29 only 1 eye had inflammation of Grade 1. Increased anterior and posterior segment inflammation (≥Grade 2) was noted in a few eyes of animals administered CR2-CR1 #7 following cessation of IM methylprednisolone on Day 35. Material was observed suspended in the vitreous in four eyes administered 1E12 or 3E12 vg/eye CR2-CR1 #7 at the Week 4 timepoint and became diffuse at subsequent timepoints in all eyes. CR2-CR1 #7 appeared to be well tolerated, with no observations of toxicity noted at any dose level on fundus ocular photography or FA.

### Microscopic Examination

Microscopic examination at the 3-month necropsy revealed minimal to slight mononuclear cell infiltrates in the eye at all dose levels and non-dose related minimal hypertrophy/hyperplasia in the RPE in a single animal at both the low and high dose.

### Biodistribution Analysis

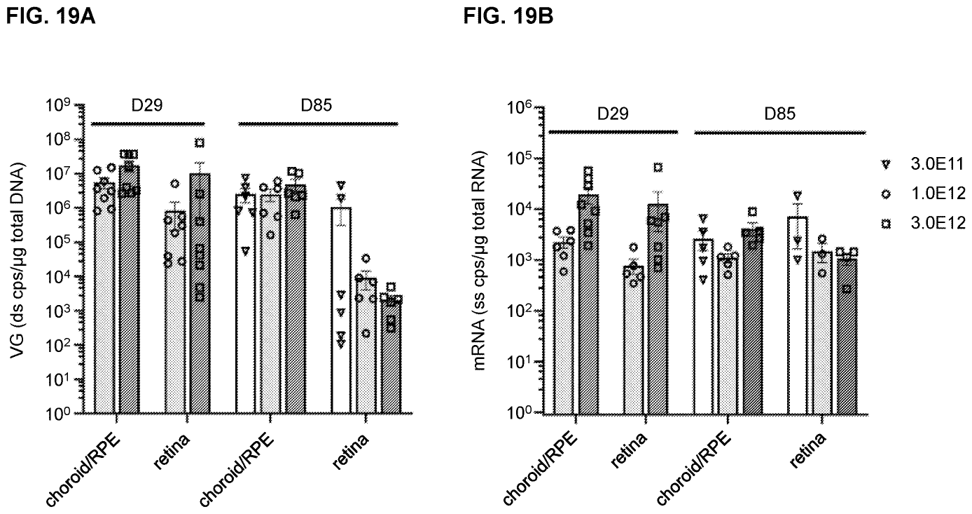

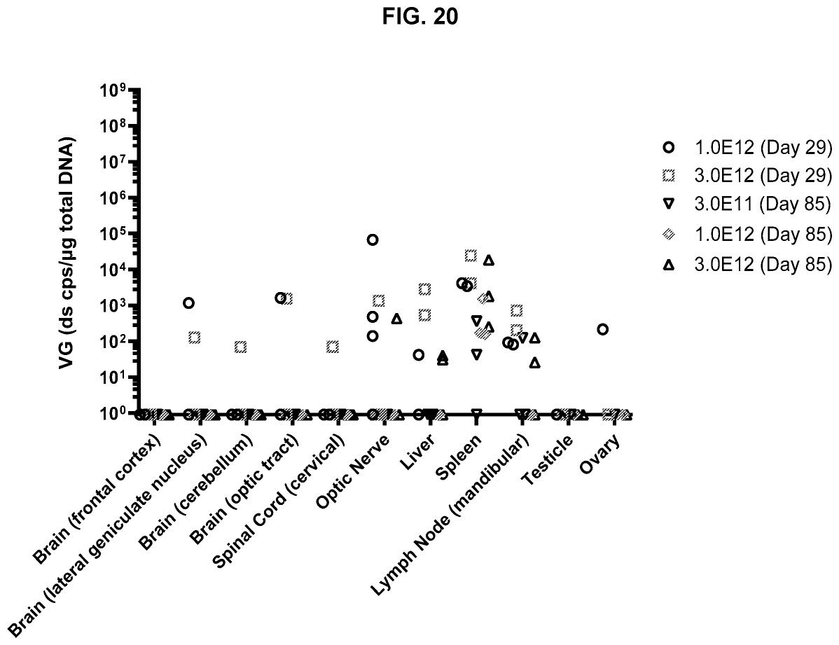

Biodistribution analysis showed that of the target ocular tissues, vector genome (vg) levels were the highest in the choroid-RPE with minimal increase across doses and consistent levels through Day 85 (FIG. 19A). This is consistent with detection of intact particles >20 nm in the ocular space >28 days after SCS injection, as clearance occurs only through the choroidal vasculature (Chiang 2016, Chiang 2018). Levels of vg in the retina were slightly lower than the choroid-RPE and decreased several logs between Day 29 and 85 (FIG. 19A). Despite reduction in vgs, mRNA expression in the retina was consistent between timepoints indicating cleared vg was non-expressing. Additionally, there was consistent mRNA expression observed across ocular tissues (retina, choroid-RPE) and between the Day 29 and 85 timepoints (FIG. 19B). Vector genomes were detected in systemic tissues in some but not all animals at levels 1 to 2 logs lower than in the eye (FIG. 20). No systemic tissues tested had quantifiable transgene mRNA. No vehicle-treated animals had detectable levels of vg or mRNA across all tissues tested.

The transgene product, CR2-CR1 #7 protein, was sporadically detected in 4/23 sampled eyes at low to mid pg/mL or pg/mg levels, in a non-dose-dependent manner (Table 11). It is noted that protein detection could be confounded since without complement activation and tethering by complement fragments, the half-life of the related CR2-CR1 fusion protein, CR2-CR1 #1 has been reported at <8 hours (Fridkis-Hareli 2019). There was no protein detection in the vitreous humor, which could be due to the ocular fluid flow, which moves away from the vitreous across the retina to the choroid. Additionally, diffusion-based molecular movement against ocular fluid flow toward the vitreous is inhibited by the same barriers that limit AAV access to the retina: the inner limiting membrane, and the RPE (Zhang 2018). Pharmacokinetic studies of SCS drug administration support short exposure to retina, choroid, and sclera, and little to no exposure to the vitreous (Chiang 2018).

TABLE 11
Incidence of Eyes with Detectable
Protein and Average Concentration

| | | | |
|---|---|---|---|
| | Group 2 | Group 3 | Group 4 |
| Group 1 | 3E11 | 1E12 | 3E12 |
| Vehicle | vg/eye | vg/eye | vg/eye |

Retina^{a}

| | | | | |
|---|---|---|---|---|
| # of eyes positive | 0/2 | 0/3 | 2/7 | 2/7 |
| for CR2-CR1 protein | | | | |
| Concentration (mean ± | N/A | N/A | 247 ± 206 | 110 ± 1.4 |
| SD, pg/mg) | | | | |

Aqueous Humor

| | | | | |
|---|---|---|---|---|
| # of eyes positive | 0/4 | 0/6 | 2/10 | 2/10 |
| for CR2-CR1 protein | | | | |
| Concentration (mean ± | N/A | N/A | 156 ± 56 | 204 ± 9 |
| SD, pg/mL) | | | | |

Vitreous Humor

| | | | | |
|---|---|---|---|---|
| # of eyes positive | 0/4 | 0/6 | 0/10 | 0/10 |
| for CR2-CR1 protein | | | | |
| Concentration (mean ± | N/A | N/A | N/A | N/A |
| SD, pg/mL) | | | | |

^{a}Protein measured unilaterally in some animals
Concentrations reflect samples collected at 4- and 12-week timepoints.
N/A = not applicable;
SD = standard deviation;
vg = vector genomes

Administration of AAV2.CR2-CR1 #7 delivered into the SCS was considered successful and well tolerated. There were no test article-related effects on clinical observations, body weight, body weight gain, food consumption, FA, ff-ERG, organ weights, or macroscopic observations at all doses and timepoints examined. Ophthalmoscopic findings included presence of material suspended in the vitreous during Week 4, which became diffuse at subsequent timepoints, and development of anterior and posterior segment intraocular inflammation (≤Grade 3) following cessation of immunosuppressant (methylprednisolone acetate) in a few eyes dosed with CR2-CR1 #7. Microscopic findings included minimal to slight mononuclear cell infiltrates (choroid, sclera, ciliary body, iris, retina, filtration angle, and/or vitreous), with minimal hypertrophy/hyperplasia of the RPE. Based on the lack of clinical effects, the stable ocular functional integrity as assessed by ERG, and the minimal severity of microscopic findings, CR2-CR1 #7 via suprachoroidal administration was considered well tolerated.

High levels of vector genomes were detected across ocular tissues with minimal systemic escape. Transgene mRNA expression in ocular tissues, despite minimal transgene protein, indicated consistent expression across timepoints.

### Example 9—Analysis of Binding to C3b, C4b, iC3b, and/or C3d Using Surface Plasmon Resonance

### Surface Plasmon Resonance (SPR) Assay

The binding of recombinant human CR1, recombinant human CR2, and recombinant CR2CR1 #1 (SEQ ID NO: 22), and recombinant CR2CR1 #7 (SEQ ID NO: 12) protein to their respective ligands C3b, C4b, iC3b, and/or C3d was analyzed using a Biacore 8K SPR system (Cytiva). Histidine-tagged complement receptors and fusion proteins were immobilized onto Series S Sensor Chip CM5 using a His Antibody Capture Kit, with capture levels of 60-470 RU. C3b, C4b, iC3b, and C3d were prepared in 1×HBS-EP buffer (10 mM HEPES, 150 mM NaCl, 3 mM EDTA, and 0.05% v/v Surfactant P20), pH 7.4, at concentrations ranging from 3-16000 nM, and the analytes were injected at a flow rate of 30 μL/min. Binding at 25° C. was monitored for 150 seconds, followed by a dissociation phase of 300 seconds. Chip sensors were regenerated for 60 seconds with 10 mM glycine-HCl buffer, pH 1.5. The data were processed and analyzed with Biacore Insight Evaluation Software; a 1:1 binding model (C3b analyte) or steady state affinity (C4b, iC3b, C3d analytes) was used to calculate the apparent K_{D} value and other kinetic parameters. Results of the SPR analysis are presented in Table 12.

TABLE 12
SPR Results

| | | | | ka | kd | KD |
|---|---|---|---|---|---|---|
| Ligand | Analyte | Analyte Conc. | | (1/Ms) | (1/s) | (M) |
| CR1 | C3b | 3.125-200 | nM | 2.33E+06 | 9.15E−03 | 3.94E−09 |
| CR2CR1 #1 | C3b | 3.125-200 | nM | 2.91E+05 | 2.32E−03 | 7.97E−09 |
| CR2CR1 #7 | C3b | 3.125-200 | nM | 1.03E+06 | 2.64E−03 | 2.56E−09 |
| CR1 | C4b | 31.25-2000 | nM | NA | NA | 3.01E−06 |
| CR2CR1 #1 | C4b | 62.5-2000 | nM | NA | NA | 2.11E−05 |
| CR2CR1 #7 | C4b | No KD obtained | | | | |
| CR2 | iC3b | 19.5-1250 | nM | NA | NA | 4.39E−07 |
| CR2CR1 #1 | iC3b | 19.5-1250 | nM | NA | NA | 4.64E−07 |
| CR2CR1 #7 | iC3b | 19.5-1250 | nM | NA | NA | 3.84E−07 |
| CR2 | C3d | 250-16000 | nM | NA | NA | 1.14E−05 |
| CR2CR1 #1 | C3d | 125-8000 | nM | NA | NA | 9.73E−06 |
| CR2CR1 #7 | C3d | 125-4000 | nM | NA | NA | 5.99E−06 |

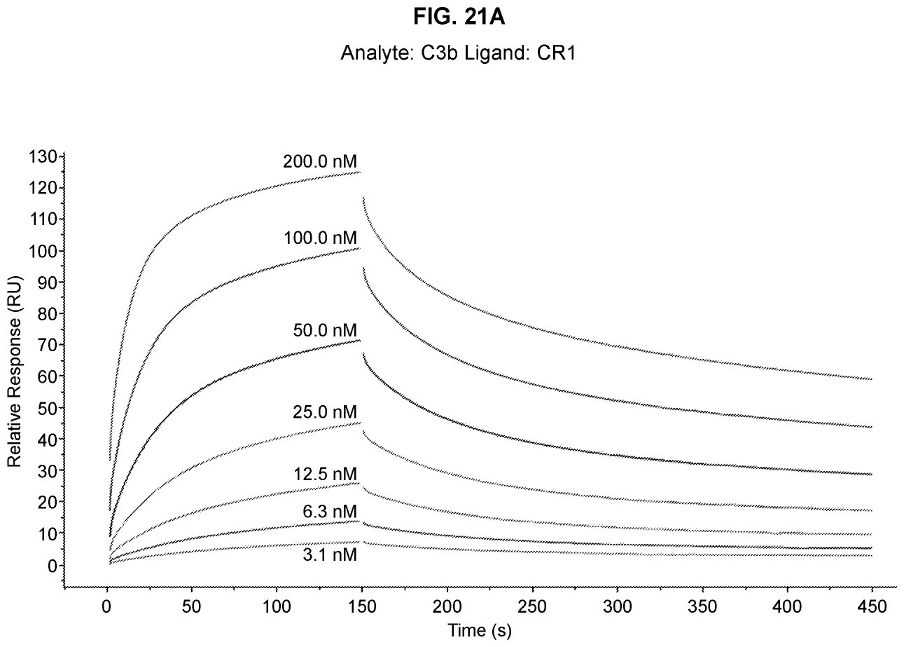

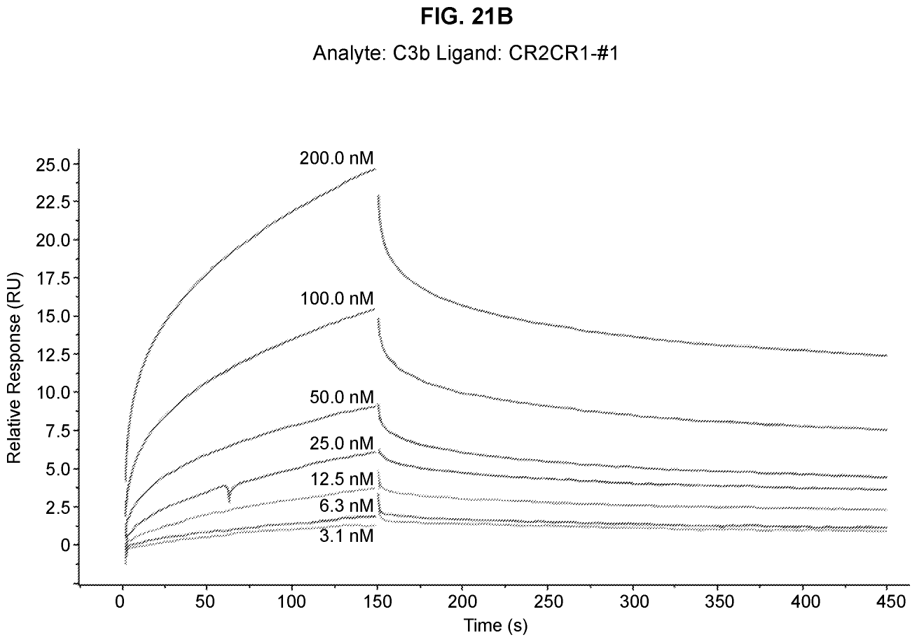

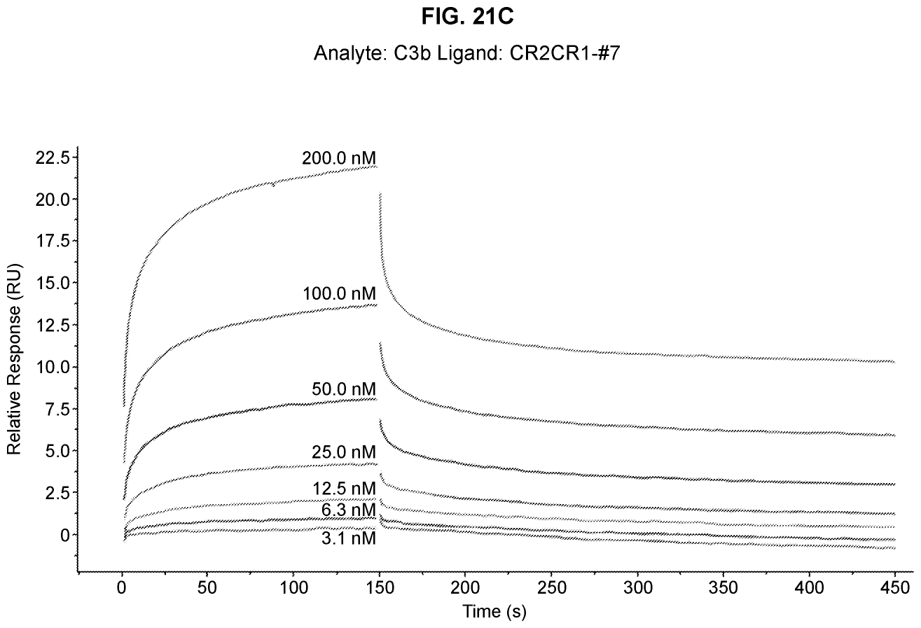

FIGS. 21A-21C show surface plasmon resonance sensograms generated with the analyte C3b, with recombinant human CR1 as the ligand (FIG. 21A), recombinant CR2CR1 #1 as the ligand (FIG. 21B), or recombinant CR2CR1 #7 as the ligand (FIG. 21C). CR2CR1 #7 demonstrated comparable or higher binding affinity to C3b, iC3b and C3d, compared to the respective complement receptor hCR1 or hCR2. In contrast, for C3b and C4b binding, CR2CR1 #1 demonstrated weaker binding than hCR1. CR2CR1 #7 exhibited approximately 3-fold higher affinity for C3b than CR2CR1 #1. Binding affinity for C4b was not obtained due to an assay failure.

Taken together, this data shows that the CR2CR1 #7 construct successfully binds to C3b, iC3b, and C3d more strongly than full-length recombinant CR1 or CR2.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

TABLE 13
Sequences:

| SEQ | De- | |
|---|---|---|
| ID | scrip- | |
| NO: | tion | Sequence |
| 1 | CR2 | ISCGSPPPILNGRISYYSTPIAVGTVIRYSCSGTFRLIGEKSLLCITKDKVDGTWDKP |
| | SCR1-4 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIF |
| 2 | CR2 | ISCGSPPPILNGRISYYSTPIAVGTVIRYSCSGTFRLIGEKSLLCITKDKVDGTWDKP |
| | SCR1-2 | APKCEYFNKYSSCPEPIVPGGYKIRGSTPYRHGDSVTFACKTNFSMNGNKSVWCQ |
| | | ANNMWGPTRLPTCVSVFP |
| 3 | CR1 | QCNAPEWLPFARPTNLTDEFEFPIGTYLNYECRPGYSGRPFSIICLKNSVWTGAKD |
| | SCR1-4, | RCRRKSCRNPPDPVNGMVHVIKGIQFGSQIKYSCTKGYRLIGSSSATCIISGDTVIW |
| | 7-11, | DNETPICDRIPCGLPPTITNGDFISTNRENFHYGSVVTYRCNPGSGGRKVFELVGEP |
| | 14-17 | SIYCTSNDDQVGIWSGPAPQCIIPNKCTPPNVENGILVSDNRSLFSLNEVVEFRCQP |
| | (CR2CR1 | GFVMKGPRRVKCQALNKWEPELPSCSRIFCPSPPVIPNGRHTGKPLEVFPFGKTVN |
| | #7) | YTCDPHPDRGTSFDLIGESTIRCTSDPQGNGVWSSPAPRCGILGHCQAPDHFLFAK |
| | | LKTQTNASDFPIGTSLKYECRPEYYGRPFSITCLDNLVWSSPKDVCKRKSCKTPPD |
| | | PVNGMVHVITDIQVGSRINYSCTTGHRLIGHSSAECILSGNAAHWSTKPPICQRIPC |
| | | GLPPTIANGDFISTNRENFHYGSVVTYRCNPGSGGRKVFELVGEPSIYCTSNDDQV |
| | | GIWSGPAPQCIIPNKCTPPNVENGILVSDNRSLFSLNEVVEFRCQPGFVMKGPRRV |
| | | KCQALNKWEPELPSCSRIFCPSPPVIPNGRHTGKPLEVFPFGKAVNYTCDPHPDRG |
| | | TSFDLIGESTIRCTSDPQGNGVWSSPAPRCGILGHCQAPDHFLFAKLKTQTNASDFP |
| | | IGTSLKYECRPEYYGRPFSITCLDNLVWSSPKDVCKRKSCKTPPDPVNGMVHVITD |
| | | IQVGSRINYSCTTGHRLIGHSSAECILSGNTAHWSTKPPICQRIPCGLPPTIANGDFIS |
| | | TNRENFHYGSVVTYRCNLGSRGRKVFELVGEPSIYCTSNDDQVGIWSGPAPQCII |
| 4 | CR1 | QCNAPEWLPFARPTNLTDEFEFPIGTYLNYECRPGYSGRPFSIICLKNSVWTGAKD |
| | SCR1-4- | RCRRKSCRNPPDPVNGMVHVIKGIQFGSQIKYSCTKGYRLIGSSSATCIISGDTVIW |
| | linker- | DNETPICDRIPCGLPPTITNGDFISTNRENFHYGSVVTYRCNPGSGGRKVFELVGEP |
| | 7-11- | SIYCTSNDDQVGIWSGPAPQCIIPNKCTPPNVENGILVSDNRSLFSLNEVVEFRCQP |
| | linker- | GFVMKGPRRVKCQALNKWEPELPSCSRGGGGSGGGGSIFCPSPPVIPNGRHTGKP |
| | 14-17 | LEVFPFGKTVNYTCDPHPDRGTSFDLIGESTIRCTSDPQGNGVWSSPAPRCGILGH |
| | (CR2CR1 | CQAPDHFLFAKLKTQTNASDFPIGTSLKYECRPEYYGRPFSITCLDNLVWSSPKDV |
| | #9) | CKRKSCKTPPDPVNGMVHVITDIQVGSRINYSCTTGHRLIGHSSAECILSGNAAHW |
| | | STKPPICQRIPCGLPPTIANGDFISTNRENFHYGSVVTYRCNPGSGGRKVFELVGEP |
| | | SIYCTSNDDQVGIWSGPAPQCIIPNKCTPPNVENGILVSDNRSLFSLNEVVEFRCQP |
| | | GFVMKGPRRVKCQALNKWEPELPSCSRGGGGSGGGGSIFCPSPPVIPNGRHTGKP |
| | | LEVFPFGKAVNYTCDPHPDRGTSFDLIGESTIRCTSDPQGNGVWSSPAPRCGILGH |
| | | CQAPDHFLFAKLKTQTNASDFPIGTSLKYECRPEYYGRPFSITCLDNLVWSSPKDV |
| | | CKRKSCKTPPDPVNGMVHVITDIQVGSRINYSCTTGHRLIGHSSAECILSGNTAHW |
| | | STKPPICQRIPCGLPPTIANGDFISTNRENFHYGSVVTYRCNLGSRGRKVFELVGEP |
| | | SIYCTSNDDQVGIWSGPAPQCII |
| 5 | CR1 | QCNAPEWLPFARPTNLTDEFEFPIGTYLNYECRPGYSGRPFSIICLKNSVWTGAKD |
| | SCR1-3- | RCRRKSCRNPPDPVNGMVHVIKGIQFGSQIKYSCTKGYRLIGSSSATCIISGDTVIW |
| | linker- | DNETPICDRIPCGLPPTITNGDFISTNRENFHYGSVVTYRCNPGSGGRKVFELVGEP |
| | 7-10- | SIYCTSNDDQVGIWSGPAPQCIIPGGGGSGGGGSGGGGSGGGGSGGGGSIFCPSPP |
| | linker- | VIPNGRHTGKPLEVFPFGKTVNYTCDPHPDRGTSFDLIGESTIRCTSDPQGNGVWS |
| | 14-17 | SPAPRCGILGHCQAPDHFLFAKLKTQTNASDFPIGTSLKYECRPEYYGRPFSITCLD |
| | (CR2CR1 | NLVWSSPKDVCKRKSCKTPPDPVNGMVHVITDIQVGSRINYSCTTGHRLIGHSSAE |
| | #10) | CILSGNAAHWSTKPPICQRIPCGLPPTIANGDFISTNRENFHYGSVVTYRCNPGSGG |
| | | RKVFELVGEPSIYCTSNDDQVGIWSGPAPQCIIPGGGGSGGGGSGGGGSGGGGSG |
| | | GGGSIFCPSPPVIPNGRHTGKPLEVFPFGKAVNYTCDPHPDRGTSFDLIGESTIRCTS |
| | | DPQGNGVWSSPAPRCGILGHCQAPDHFLFAKLKTQTNASDFPIGTSLKYECRPEY |
| | | YGRPFSITCLDNLVWSSPKDVCKRKSCKTPPDPVNGMVHVITDIQVGSRINYSCTT |
| | | GHRLIGHSSAECILSGNTAHWSTKPPICQRIPCGLPPTIANGDFISTNRENFHYGSVV |
| | | TYRCNLGSRGRKVFELVGEPSIYCTSNDDQVGIWSGPAPQCII |
| 6 | CR1 | QCNAPEWLPFARPTNLTDEFEFPIGTYLNYECRPGYSGRPFSIICLKNSVWTGAKD |
| | SCR1-3- | RCRRKSCRNPPDPVNGMVHVIKGIQFGSQIKYSCTKGYRLIGSSSATCIISGDTVIW |
| | linker- | DNETPICDRIPCGLPPTITNGDFISTNRENFHYGSVVTYRCNPGSGGRKVFELVGEP |
| | 7-10- | SIYCTSNDDQVGIWSGPAPQCIIPGGGGSGGGGSGGGGSGGGGSGGGGSGGGGSG |
| | linker- | GGGSGGGGGGGGSGGGGSGGGGSGGGGSIFCPSPPVIPNGRHTGKPLEVFPFGK |
| | 14-17 | TVNYTCDPHPDRGTSFDLIGESTIRCTSDPQGNGVWSSPAPRCGILGHCQAPDHFL |
| | (CR2CR1 | FAKLKTQTNASDFPIGTSLKYECRPEYYGRPFSITCLDNLVWSSPKDVCKRKSCKT |
| | #10) | PPDPVNGMVHVITDIQVGSRINYSCTTGHRLIGHSSAECILSGNAAHWSTKPPICQR |
| | | IPCGLPPTIANGDFISTNRENFHYGSVVTYRCNPGSGGRKVFELVGEPSIYCTSNDD |
| | | QVGIWSGPAPQCIIPGGGGSGGGGSGGGGSGGGGSGGGGSGGGGSGGGGSGGGG |
| | | SGGGGSGGGGSGGGGSGGGGSIFCPSPPVIPNGRHTGKPLEVFPFGKAVNYTCDP |
| | | HPDRGTSFDLIGESTIRCTSDPQGNGVWSSPAPRCGILGHCQAPDHFLFAKLKTQT |
| | | NASDFPIGTSLKYECRPEYYGRPFSITCLDNLVWSSPKDVCKRKSCKTPPDPVNGM |
| | | VHVITDIQVGSRINYSCTTGHRLIGHSSAECILSGNTAHWSTKPPICQRIPCGLPPTI |
| | | ANGDFISTNRENFHYGSVVTYRCNLGSRGRKVFELVGEPSIYCTSNDDQVGIWSG |
| | | PAPQCII |
| 7 | CR1 | QCNAPEWLPFARPTNLTDEFEFPIGTYLNYECRPGYSGRPFSIICLKNSVWTGAKD |
| | SCR1-4, | RCRRKSCRNPPDPVNGMVHVIKGIQFGSQIKYSCTKGYRLIGSSSATCIISGDTVIW |
| | 6-12, | DNETPICDRIPCGLPPTITNGDFISTNRENFHYGSVVTYRCNPGSGGRKVFELVGEP |
| | 14-17 | SIYCTSNDDQVGIWSGPAPQCIIPNKCTPPNVENGILVSDNRSLFSLNEVVEFRCQP |
| | (CR2CR1 | GFVMKGPRRVKCQALNKWEPELPSCSRVCQPPPDVLHAERTQRDKDNFSPGQEV |
| | #12) | FYSCEPGYDLRGAASMRCTPQGDWSPAAPTCEVIFCPSPPVIPNGRHTGKPLEVFP |
| | | FGKTVNYTCDPHPDRGTSFDLIGESTIRCTSDPQGNGVWSSPAPRCGILGHCQAPD |
| | | HFLFAKLKTQTNASDFPIGTSLKYECRPEYYGRPFSITCLDNLVWSSPKDVCKRKS |
| | | CKTPPDPVNGMVHVITDIQVGSRINYSCTTGHRLIGHSSAECILSGNAAHWSTKPPI |
| | | CQRIPCGLPPTIANGDFISTNRENFHYGSVVTYRCNPGSGGRKVFELVGEPSIYCTS |
| | | NDDQVGIWSGPAPQCIIPNKCTPPNVENGILVSDNRSLFSLNEVVEFRCQPGFVMK |
| | | GPRRVKCQALNKWEPELPSCSRVCQPPPDVLHAERTQRDKDNFSPGQEVFYSCEP |
| | | GYDLRGAASMRCTPQGDWSPAAPTCEVIFCPSPPVIPNGRHTGKPLEVFPFGKAV |
| | | NYTCDPHPDRGTSFDLIGESTIRCTSDPQGNGVWSSPAPRCGILGHCQAPDHFLFA |
| | | KLKTQTNASDFPIGTSLKYECRPEYYGRPFSITCLDNLVWSSPKDVCKRKSCKTPP |
| | | DPVNGMVHVITDIQVGSRINYSCTTGHRLIGHSSAECILSGNTAHWSTKPPICQRIP |
| | | CGLPPTIANGDFISTNRENFHYGSVVTYRCNLGSRGRKVFELVGEPSIYCTSNDDQ |
| | | VGIWSGPAPQCII |
| 8 | CR1 | QCNAPEWLPFARPTNLTDEFEFPIGTYLNYECRPGYSGRPFSIICLKNSVWTGAKD |
| | SCR1-10 | RCRRKSCRNPPDPVNGMVHVIKGIQFGSQIKYSCTKGYRLIGSSSATCIISGDTVIW |
| | (CR2CR1 | DNETPICDRIPCGLPPTITNGDFISTNRENFHYGSVVTYRCNPGSGGRKVFELVGEP |
| | #1) | SIYCTSNDDQVGIWSGPAPQCIIPNKCTPPNVENGILVSDNRSLFSLNEVVEFRCQP |
| | | GFVMKGPRRVKCQALNKWEPELPSCSRVCQPPPDVLHAERTQRDKDNFSPGQEV |
| | | FYSCEPGYDLRGAASMRCTPQGDWSPAAPTCEVKSCDDFMGQLLNGRVLFPVNL |
| | | QLGAKVDFVCDEGFQLKGSSASYCVLAGMESLWNSSVPVCEQIFCPSPPVIPNGR |
| | | HTGKPLEVFPFGKTVNYTCDPHPDRGTSFDLIGESTIRCTSDPQGNGVWSSPAPRC |
| | | GILGHCQAPDHFLFAKLKTQTNASDFPIGTSLKYECRPEYYGRPFSITCLDNLVWS |
| | | SPKDVCKRKSCKTPPDPVNGMVHVITDIQVGSRINYSCTTGHRLIGHSSAECILSG |
| | | NAAHWSTKPPICQRIPCGLPPTIANGDFISTNRENFHYGSVVTYRCNPGSGGRKVF |
| | | ELVGEPSIYCTSNDDQVGIWSGPAPQCIIPNK |
| 9 | CR1 | QCNAPEWLPFARPTNLTDEFEFPIGTYLNYECRPGYSGRPFSIICLKNSVWTGAKD |
| | SCR1-3, | RCRRKSCRNPPDPVNGMVHVIKGIQFGSQIKYSCTKGYRLIGSSSATCIISGDTVIW |
| | 8-10, | DNETPICDRIPCGLPPTITNGDFISTNRENFHYGSVVTYRCNPGSGGRKVFELVGEP |
| | 15-17 | SIYCTSNDDQVGIWSGPAPQCIIGHCQAPDHFLFAKLKTQTNASDFPIGTSLKYECR |
| | (CR2CR1 | PEYYGRPFSITCLDNLVWSSPKDVCKRKSCKTPPDPVNGMVHVITDIQVGSRINYS |
| | #3) | CTTGHRLIGHSSAECILSGNAAHWSTKPPICQRIPCGLPPTIANGDFISTNRENFHYG |
| | | SVVTYRCNPGSGGRKVFELVGEPSIYCTSNDDQVGIWSGPAPQCIIPNKGHCQAPD |
| | | HFLFAKLKTQTNASDFPIGTSLKYECRPEYYGRPFSITCLDNLVWSSPKDVCKRKS |
| | | CKTPPDPVNGMVHVITDIQVGSRINYSCTTGHRLIGHSSAECILSGNTAHWSTKPPI |
| | | CQRIPCGLPPTIANGDFISTNRENFHYGSVVTYRCNLGSRGRKVFELVGEPSIYCTS |
| | | NDDQVGIWSGPAPQCII |
| 10 | CR1 | QCNAPEWLPFARPTNLTDEFEFPIGTYLNYECRPGYSGRPFSIICLKNSVWTGAKD |
| | SCR1-14 | RCRRKSCRNPPDPVNGMVHVIKGIQFGSQIKYSCTKGYRLIGSSSATCIISGDTVIW |
| | (CR2CR1 | DNETPICDRIPCGLPPTITNGDFISTNRENFHYGSVVTYRCNPGSGGRKVFELVGEP |
| | #1) | SIYCTSNDDQVGIWSGPAPQCIIPNKCTPPNVENGILVSDNRSLFSLNEVVEFRCQP |
| | | GFVMKGPRRVKCQALNKWEPELPSCSRVCQPPPDVLHAERTQRDKDNFSPGQEV |
| | | FYSCEPGYDLRGAASMRCTPQGDWSPAAPTCEVKSCDDFMGQLLNGRVLFPVNL |
| | | QLGAKVDFVCDEGFQLKGSSASYCVLAGMESLWNSSVPVCEQIFCPSPPVIPNGR |
| | | HTGKPLEVFPFGKTVNYTCDPHPDRGTSFDLIGESTIRCTSDPQGNGVWSSPAPRC |
| | | GILGHCQAPDHFLFAKLKTQTNASDFPIGTSLKYECRPEYYGRPFSITCLDNLVWS |
| | | SPKDVCKRKSCKTPPDPVNGMVHVITDIQVGSRINYSCTTGHRLIGHSSAECILSG |
| | | NAAHWSTKPPICQRIPCGLPPTIANGDFISTNRENFHYGSVVTYRCNPGSGGRKVF |
| | | ELVGEPSIYCTSNDDQVGIWSGPAPQCIIPNKCTPPNVENGILVSDNRSLFSLNEVV |
| | | EFRCQPGFVMKGPRRVKCQALNKWEPELPSCSRVCQPPPDVLHAERTQRDKDNF |
| | | SPGQEVFYSCEPGYDLRGAASMRCTPQGDWSPAAPTCEVKSCDDFMGQLLNGRV |
| | | LFPVNLQLGAKVDFVCDEGFQLKGSSASYCVLAGMESLWNSSVPVCEQIFCPSPP |
| | | VIPNGRHTGKPLEVFPFGKAVNYTCDPHPDRGTSFDLIGESTIRCTSDPQGNGVWS |
| | | SPAPRCGI |
| 11 | CR1 | QCNAPEWLPFARPTNLTDEFEFPIGTYLNYECRPGYSGRPFSIICLKNSVWTGAKD |
| | SCR1-17 | RCRRKSCRNPPDPVNGMVHVIKGIQFGSQIKYSCTKGYRLIGSSSATCIISGDTVIW |
| | (CR2CR1 | DNETPICDRIPCGLPPTITNGDFISTNRENFHYGSVVTYRCNPGSGGRKVFELVGEP |
| | #13) | SIYCTSNDDQVGIWSGPAPQCIIPNKCTPPNVENGILVSDNRSLFSLNEVVEFRCQP |
| | | GFVMKGPRRVKCQALNKWEPELPSCSRVCQPPPDVLHAERTQRDKDNFSPGQEV |
| | | FYSCEPGYDLRGAASMRCTPQGDWSPAAPTCEVKSCDDFMGQLLNGRVLFPVNL |
| | | QLGAKVDFVCDEGFQLKGSSASYCVLAGMESLWNSSVPVCEQIFCPSPPVIPNGR |
| | | HTGKPLEVFPFGKTVNYTCDPHPDRGTSFDLIGESTIRCTSDPQGNGVWSSPAPRC |
| | | GILGHCQAPDHFLFAKLKTQTNASDFPIGTSLKYECRPEYYGRPFSITCLDNLVWS |
| | | SPKDVCKRKSCKTPPDPVNGMVHVITDIQVGSRINYSCTTGHRLIGHSSAECILSG |
| | | NAAHWSTKPPICQRIPCGLPPTIANGDFISTNRENFHYGSVVTYRCNPGSGGRKVF |
| | | ELVGEPSIYCTSNDDQVGIWSGPAPQCIIPNKCTPPNVENGILVSDNRSLFSLNEVV |
| | | EFRCQPGFVMKGPRRVKCQALNKWEPELPSCSRVCQPPPDVLHAERTQRDKDNF |
| | | SPGQEVFYSCEPGYDLRGAASMRCTPQGDWSPAAPTCEVKSCDDFMGQLLNGRV |
| | | LFPVNLQLGAKVDFVCDEGFQLKGSSASYCVLAGMESLWNSSVPVCEQIFCPSPP |
| | | VIPNGRHTGKPLEVFPFGKAVNYTCDPHPDRGTSFDLIGESTIRCTSDPQGNGVWS |
| | | SPAPRCGILGHCQAPDHFLFAKLKTQTNASDFPIGTSLKYECRPEYYGRPFSITCLD |
| | | NLVWSSPKDVCKRKSCKTPPDPVNGMVHVITDIQVGSRINYSCTTGHRLIGHSSAE |
| | | CILSGNTAHWSTKPPICQRIPCGLPPTIANGDFISTNRENFHYGSVVTYRCNLGSRG |
| | | RKVFELVGEPSIYCTSNDDQVGIWSGPAPQCII |
| 12 | CR2CR1 | ISCGSPPPILNGRISYYSTPIAVGTVIRYSCSGTFRLIGEKSLLCITKDKVDGTWDKP |
| | #7 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFQCNAPEWLPFARPTNLTDEFEFP |
| | | IGTYLNYECRPGYSGRPFSIICLKNSVWTGAKDRCRRKSCRNPPDPVNGMVH |
| | | VIKGIQFGSQIKYSCTKGYRLIGSSSATCIISGDTVIWDNETPICDRIPCGLPPTI |
| | | TNGDFISTNRENFHYGSVVTYRCNPGSGGRKVFELVGEPSIYCTSNDDQVGIW |
| | | SGPAPQCIIPNKCTPPNVENGILVSDNRSLFSLNEVVEFRCQPGFVMKGPRRV |
| | | KCQALNKWEPELPSCSRIFCPSPPVIPNGRHTGKPLEVFPFGKTVNYTCDPHP |
| | | DRGTSFDLIGESTIRCTSDPQGNGVWSSPAPRCGILGHCQAPDHFLFAKLKTQ |
| | | TNASDFPIGTSLKYECRPEYYGRPFSITCLDNLVWSSPKDVCKRKSCKTPPDP |
| | | VNGMVHVITDIQVGSRINYSCTTGHRLIGHSSAECILSGNAAHWSTKPPICQRI |
| | | PCGLPPTIANGDFISTNRENFHYGSVVTYRCNPGSGGRKVFELVGEPSIYCTSN |
| | | DDQVGIWSGPAPQCIIPNKCTPPNVENGILVSDNRSLFSLNEVVEFRCQPGFV |
| | | MKGPRRVKCQALNKWEPELPSCSRIFCPSPPVIPNGRHTGKPLEVFPFGKAV |
| | | NYTCDPHPDRGTSFDLIGESTIRCTSDPQGNGVWSSPAPRCGILGHCQAPDHF |
| | | LFAKLKTQTNASDFPIGTSLKYECRPEYYGRPFSITCLDNLVWSSPKDVCKRK |
| | | SCKTPPDPVNGMVHVITDIQVGSRINYSCTTGHRLIGHSSAECILSGNTAHWS |
| | | TKPPICQRIPCGLPPTIANGDFISTNRENFHYGSVVTYRCNLGSRGRKVFELVG |
| | | EPSIYCTSNDDQVGIWSGPAPQCII |
| 13 | CR2CR1 | ISCGSPPPILNGRISYYSTPIAVGTVIRYSCSGTFRLIGEKSLLCITKDKVDGTWDKP |
| | #8 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFGGGGSGGGGSQCNAPEWLPFARP |
| | | TNLTDEFEFPIGTYLNYECRPGYSGRPFSIICLKNSVWTGAKDRCRRKSCRNP |
| | | PDPVNGMVHVIKGIQFGSQIKYSCTKGYRLIGSSSATCIISGDTVIWDNETPIC |
| | | DRIPCGLPPTITNGDFISTNRENFHYGSVVTYRCNPGSGGRKVFELVGEPSIYC |
| | | TSNDDQVGIWSGPAPQCIIPNKCTPPNVENGILVSDNRSLFSLNEVVEFRCQPG |
| | | FVMKGPRRVKCQALNKWEPELPSCSRIFCPSPPVIPNGRHTGKPLEVFPFGKT |
| | | VNYTCDPHPDRGTSFDLIGESTIRCTSDPQGNGVWSSPAPRCGILGHCQAPDH |
| | | FLFAKLKTQTNASDFPIGTSLKYECRPEYYGRPFSITCLDNLVWSSPKDVCKR |
| | | KSCKTPPDPVNGMVHVITDIQVGSRINYSCTTGHRLIGHSSAECILSGNAAHW |
| | | STKPPICQRIPCGLPPTIANGDFISTNRENFHYGSVVTYRCNPGSGGRKVFELV |
| | | GEPSIYCTSNDDQVGIWSGPAPQCHIPNKCTPPNVENGILVSDNRSLFSLNEVV |
| | | EFRCQPGFVMKGPRRVKCQALNKWEPELPSCSRIFCPSPPVIPNGRHTGKPL |
| | | EVFPFGKAVNYTCDPHPDRGTSFDLIGESTIRCTSDPQGNGVWSSPAPRCGIL |
| | | GHCQAPDHFLFAKLKTQTNASDFPIGTSLKYECRPEYYGRPFSITCLDNLVWS |
| | | SPKDVCKRKSCKTPPDPVNGMVHVITDIQVGSRINYSCTTGHRLIGHSSAECI |
| | | LSGNTAHWSTKPPICQRIPCGLPPTIANGDFISTNRENFHYGSVVTYRCNLGSR |
| | | GRKVFELVGEPSIYCTSNDDQVGIWSGPAPQCII |
| 14 | CR2CR1 | ISCGSPPPILNGRISYYSTPIAVGTVIRYSCSGTFRLIGEKSLLCITKDKVDGTWDKP |
| | #9 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFGGGGSGGGGSQCNAPEWLPFARP |
| | | TNLTDEFEFPIGTYLNYECRPGYSGRPFSIICLKNSVWTGAKDRCRRKSCRNP |
| | | PDPVNGMVHVIKGIQFGSQIKYSCTKGYRLIGSSSATCIISGDTVIWDNETPIC |
| | | DRIPCGLPPTITNGDFISTNRENFHYGSVVTYRCNPGSGGRKVFELVGEPSIYC |
| | | TSNDDQVGIWSGPAPQCIIPNKCTPPNVENGILVSDNRSLFSLNEVVEFRCQPG |
| | | FVMKGPRRVKCQALNKWEPELPSCSRGGGGSGGGGSIFCPSPPVIPNGRHTG |
| | | KPLEVFPFGKTVNYTCDPHPDRGTSFDLIGESTIRCTSDPQGNGVWSSPAPRC |
| | | GILGHCQAPDHFLFAKLKTQTNASDFPIGTSLKYECRPEYYGRPFSITCLDNL |
| | | VWSSPKDVCKRKSCKTPPDPVNGMVHVITDIQVGSRINYSCTTGHRLIGHSSA |
| | | ECILSGNAAHWSTKPPICQRIPCGLPPTIANGDFISTNRENFHYGSVVTYRCNP |
| | | GSGGRKVFELVGEPSIYCTSNDDQVGIWSGPAPQCIIPNKCTPPNVENGILVSD |
| | | NRSLFSLNEVVEFRCQPGFVMKGPRRVKCQALNKWEPELPSCSRGGGGSGGG |
| | | GSIFCPSPPVIPNGRHTGKPLEVFPFGKAVNYTCDPHPDRGTSFDLIGESTIRCT |
| | | SDPQGNGVWSSPAPRCGILGHCQAPDHFLFAKLKTQTNASDFPIGTSLKYEC |
| | | RPEYYGRPFSITCLDNLVWSSPKDVCKRKSCKTPPDPVNGMVHVITDIQVGSR |
| | | INYSCTTGHRLIGHSSAECILSGNTAHWSTKPPICQRIPCGLPPTIANGDFISTN |
| | | RENFHYGSVVTYRCNLGSRGRKVFELVGEPSIYCTSNDDQVGIWSGPAPQCII |
| 15 | CR2CR1 | ISCGSPPPILNGRISYYSTPIAVGTVIRYSCSGTFRLIGEKSLLCITKDKVDGTWDKP |
| | #10 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFQCNAPEWLPFARPTNLTDEFEFP |
| | | IGTYLNYECRPGYSGRPFSIICLKNSVWTGAKDRCRRKSCRNPPDPVNGMVH |
| | | VIKGIQFGSQIKYSCTKGYRLIGSSSATCIISGDTVIWDNETPICDRIPCGLPPTI |
| | | TNGDFISTNRENFHYGSVVTYRCNPGSGGRKVFELVGEPSIYCTSNDDQVGIW |
| | | SGPAPQCIIPGGGGSGGGGSGGGGSGGGGSGGGGSGGGGSGGGGSGGGGSGGGG |
| | | SGGGGSGGGGSGGGGSIFCPSPPVIPNGRHTGKPLEVFPFGKTVNYTCDPHPDR |
| | | GTSFDLIGESTIRCTSDPQGNGVWSSPAPRCGILGHCQAPDHFLFAKLKTQTN |
| | | ASDFPIGTSLKYECRPEYYGRPFSITCLDNLVWSSPKDVCKRKSCKTPPDPVN |
| | | GMVHVITDIQVGSRINYSCTTGHRLIGHSSAECILSGNAAHWSTKPPICQRIPC |
| | | GLPPTIANGDFISTNRENFHYGSVVTYRCNPGSGGRKVFELVGEPSIYCTSND |
| | | DQVGIWSGPAPQCIIPGGGGSGGGGSGGGGSGGGGSGGGGSGGGGSGGGGSGG |
| | | GGSGGGGSGGGGSGSGGGGSIFCPSPPVIPNGRHTGKPLEVFPFGKAVNYT |
| | | CDPHPDRGTSFDLIGESTIRCTSDPQGNGVWSSPAPRCGILGHCQAPDHFLFA |
| | | KLKTQTNASDFPIGTSLKYECRPEYYGRPFSITCLDNLVWSSPKDVCKRKSCK |
| | | TPPDPVNGMVHVITDIQVGSRINYSCTTGHRLIGHSSAECILSGNTAHWSTKP |
| | | PICQRIPCGLPPTIANGDFISTNRENFHYGSVVTYRCNLGSRGRKVFELVGEPS |
| | | IYCTSNDDQVGIWSGPAPQCII |
| 16 | CR2CR1 | ISCGSPPPILNGRISYYSTPIAVGTVIRYSCSGTFRLIGEKSLLCITKDKVDGTWDKP |
| | #10- | APKCEYFNKYSSCPEPIVPGGYKIRGSTPYRHGDSVTFACKTNFSMNGNKSVWCQ |
| | (G4S)5 | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFQCNAPEWLPFARPTNLTDEFEFP |
| | | IGTYLNYECRPGYSGRPFSIICLKNSVWTGAKDRCRRKSCRNPPDPVNGMVH |
| | | VIKGIQFGSQIKYSCTKGYRLIGSSSATCIISGDTVIWDNETPICDRIPCGLPPTI |
| | | TNGDFISTNRENFHYGSVVTYRCNPGSGGRKVFELVGEPSIYCTSNDDQVGIW |
| | | SGPAPQCIIPGGGGSGGGGSGGGGSGGGGSGGGGSIFCPSPPVIPNGRHTGKPLE |
| | | VFPFGKTVNYTCDPHPDRGTSFDLIGESTIRCTSDPQGNGVWSSPAPRCGILG |
| | | HCQAPDHFLFAKLKTQTNASDFPIGTSLKYECRPEYYGRPFSITCLDNLVWSS |
| | | PKDVCKRKSCKTPPDPVNGMVHVITDIQVGSRINYSCTTGHRLIGHSSAECIL |
| | | SGNAAHWSTKPPICQRIPCGLPPTIANGDFISTNRENFHYGSVVTYRCNPGSG |
| | | GRKVFELVGEPSIYCTSNDDQVGIWSGPAPQCHIPGGGGSGGGGSGGGGSGGG |
| | | GSGGGGSIFCPSPPVIPNGRHTGKPLEVFPFGKAVNYTCDPHPDRGTSFDLIGE |
| | | STIRCTSDPQGNGVWSSPAPRCGILGHCQAPDHFLFAKLKTQTNASDFPIGTS |
| | | LKYECRPEYYGRPFSITCLDNLVWSSPKDVCKRKSCKTPPDPVNGMVHVITDI |
| | | QVGSRINYSCTTGHRLIGHSSAECILSGNTAHWSTKPPICQRIPCGLPPTIANG |
| | | DFISTNRENFHYGSVVTYRCNLGSRGRKVFELVGEPSIYCTSNDDQVGIWSGP |
| | | APQCII |
| 17 | CR2CR1 | ISCGSPPPILNGRISYYSTPIAVGTVIRYSCSGTFRLIGEKSLLCITKDKVDGTWDKP |
| | #11 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFGGGGSGGGGSQCNAPEWLPFARP |
| | | TNLTDEFEFPIGTYLNYECRPGYSGRPFSIICLKNSVWTGAKDRCRRKSCRNP |
| | | PDPVNGMVHVIKGIQFGSQIKYSCTKGYRLIGSSSATCIISGDTVIWDNETPIC |
| | | DRIPCGLPPTITNGDFISTNRENFHYGSVVTYRCNPGSGGRKVFELVGEPSIYC |
| | | TSNDDQVGIWSGPAPQCIIPGGGGSGGGGSGGGGSGGGGSGGGGSGGGGSGGG |
| | | GSGGGGSGGGGSGGGGSGGGGSGGGGSIFCPSPPVIPNGRHTGKPLEVFPFGKT |
| | | VNYTCDPHPDRGTSFDLIGESTIRCTSDPQGNGVWSSPAPRCGILGHCQAPDH |
| | | FLFAKLKTQTNASDFPIGTSLKYECRPEYYGRPFSITCLDNLVWSSPKDVCKR |
| | | KSCKTPPDPVNGMVHVITDIQVGSRINYSCTTGHRLIGHSSAECILSGNAAHW |
| | | STKPPICQRIPCGLPPTIANGDFISTNRENFHYGSVVTYRCNPGSGGRKVFELV |
| | | GEPSIYCTSNDDQVGIWSGPAPQCIIPGGGGSGGGGSGGGGSGGGGSGGGGSGG |
| | | GGSGGGGSGGGGSGCGGSGGGGSGGGGSGGGGSIFCPSPPVIPNGRHTGKPLEV |
| | | FPFGKAVNYTCDPHPDRGTSFDLIGESTIRCTSDPQGNGVWSSPAPRCGILGH |
| | | CQAPDHFLFAKLKTQTNASDFPIGTSLKYECRPEYYGRPFSITCLDNLVWSSP |
| | | KDVCKRKSCKTPPDPVNGMVHVITDIQVGSRINYSCTTGHRLIGHSSAECILS |
| | | GNTAHWSTKPPICQRIPCGLPPTIANGDFISTNRENFHYGSVVTYRCNLGSRG |
| | | RKVFELVGEPSIYCTSNDDQVGIWSGPAPQCII |
| 18 | CR2CR1 | ISCGSPPPILNGRISYYSTPIAVGTVIRYSCSGTFRLIGEKSLLCITKDKVDGTWDKP |
| | #11- | APKCEYFNKYSSCPEPIVPGGYKIRGSTPYRHGDSVTFACKTNFSMNGNKSVWCQ |
| | (G4S)5 | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFGGGGSGGGGSQCNAPEWLPFARP |
| | | TNLTDEFEFPIGTYLNYECRPGYSGRPFSIICLKNSVWTGAKDRCRRKSCRNP |
| | | PDPVNGMVHVIKGIQFGSQIKYSCTKGYRLIGSSSATCIISGDTVIWDNETPIC |
| | | DRIPCGLPPTITNGDFISTNRENFHYGSVVTYRCNPGSGGRKVFELVGEPSIYC |
| | | TSNDDQVGIWSGPAPQCIIPGGGGSGGGGSGGGGSGGGGSGGGGSIFCPSPPVIP |
| | | NGRHTGKPLEVFPFGKTVNYTCDPHPDRGTSFDLIGESTIRCTSDPQGNGVW |
| | | SSPAPRCGILGHCQAPDHFLFAKLKTQTNASDFPIGTSLKYECRPEYYGRPFSI |
| | | TCLDNLVWSSPKDVCKRKSCKTPPDPVNGMVHVITDIQVGSRINYSCTTGHR |
| | | LIGHSSAECILSGNAAHWSTKPPICQRIPCGLPPTIANGDFISTNRENFHYGSVV |
| | | TYRCNPGSGGRKVFELVGEPSIYCTSNDDQVGIWSGPAPQCIIPGGGGSGGGG |
| | | SGGGGSGGGGSGGGGSIFCPSPPVIPNGRHTGKPLEVFPFGKAVNYTCDPHPDR |
| | | GTSFDLIGESTIRCTSDPQGNGVWSSPAPRCGILGHCQAPDHFLFAKLKTQTN |
| | | ASDFPIGTSLKYECRPEYYGRPFSITCLDNLVWSSPKDVCKRKSCKTPPDPVN |
| | | GMVHVITDIQVGSRINYSCTTGHRLIGHSSAECILSGNTAHWSTKPPICQRIPC |
| | | GLPPTIANGDFISTNRENFHYGSVVTYRCNLGSRGRKVFELVGEPSIYCTSND |
| | | DQVGIWSGPAPQCII |
| 19 | CR2CR1 | ISCGSPPPILNGRISYYSTPIAVGTVIRYSCSGTFRLIGEKSLLCITKDKVDGTWDKP |
| | #12 | APKCEYFNKYSSCPEPIVPGGYKIRGSTPYRHGDSVTFACKTNFSMNGNKSVWCQ |
| | | ANNMWGPTRLPTCVSVFPGGSGGGGSGGGGSGGGGSGGGGSQCNAP |
| | | EWLPFARPTNLTDEFEFPIGTYLNYECRPGYSGRPFSIICLKNSVWTGAKDRC |
| | | RRKSCRNPPDPVNGMVHVIKGIQFGSQIKYSCTKGYRLIGSSSATCIISGDTVI |
| | | WDNETPICDRIPCGLPPTITNGDFISTNRENFHYGSVVTYRCNPGSGGRKVFE |
| | | LVGEPSIYCTSNDDQVGIWSGPAPQCHIPNKCTPPNVENGILVSDNRSLFSLNE |
| | | VVEFRCQPGFVMKGPRRVKCQALNKWEPELPSCSRVCQPPPDVLHAERTQR |
| | | DKDNFSPGQEVFYSCEPGYDLRGAASMRCTPQGDWSPAAPTCEVIFCPSPPVI |
| | | PNGRHTGKPLEVFPFGKTVNYTCDPHPDRGTSFDLIGESTIRCTSDPQGNGV |
| | | WSSPAPRCGILGHCQAPDHFLFAKLKTQTNASDFPIGTSLKYECRPEYYGRPF |
| | | SITCLDNLVWSSPKDVCKRKSCKTPPDPVNGMVHVITDIQVGSRINYSCTTGH |
| | | RLIGHSSAECILSGNAAHWSTKPPICQRIPCGLPPTIANGDFISTNRENFHYGSV |
| | | VTYRCNPGSGGRKVFELVGEPSIYCTSNDDQVGIWSGPAPQCIIPNKCTPPNV |
| | | ENGILVSDNRSLFSLNEVVEFRCQPGFVMKGPRRVKCQALNKWEPELPSCSR |
| | | VCQPPPDVLHAERTQRDKDNFSPGQEVFYSCEPGYDLRGAASMRCTPQGDW |
| | | SPAAPTCEVIFCPSPPVIPNGRHTGKPLEVFPFGKAVNYTCDPHPDRGTSFDLI |
| | | GESTIRCTSDPQGNGVWSSPAPRCGILGHCQAPDHFLFAKLKTQTNASDFPIG |
| | | TSLKYECRPEYYGRPFSITCLDNLVWSSPKDVCKRKSCKTPPDPVNGMVHVI |
| | | TDIQVGSRINYSCTTGHRLIGHSSAECILSGNTAHWSTKPPICQRIPCGLPPTIA |
| | | NGDFISTNRENFHYGSVVTYRCNLGSRGRKVFELVGEPSIYCTSNDDQVGIWS |
| | | GPAPQCII |
| 20 | CR2CR1 | ISCGSPPPILNGRISYYSTPIAVGTVIRYSCSGTFRLIGEKSLLCITKDKVDGTWDKP |
| | #12- | APKCEYFNKYSSCPEPIVPGGYKIRGSTPYRHGDSVTFACKTNFSMNGNKSVWCQ |
| | (G4S)3 | ANNMWGPTRLPTCVSVFPGGGGSGGGGSGGGGSQCNAPEWLPFARPTNLTDEF |
| | | EFPIGTYLNYECRPGYSGRPFSIICLKNSVWTGAKDRCRRKSCRNPPDPVNGM |
| | | VHVIKGIQFGSQIKYSCTKGYRLIGSSSATCIISGDTVIWDNETPICDRIPCGLP |
| | | PTITNGDFISTNRENFHYGSVVTYRCNPGSGGRKVFELVGEPSIYCTSNDDQV |
| | | GIWSGPAPQCIIPNKCTPPNVENGILVSDNRSLFSLNEVVEFRCQPGFVMKGP |
| | | RRVKCQALNKWEPELPSCSRVCQPPPDVLHAERTQRDKDNFSPGQEVFYSCE |
| | | PGYDLRGAASMRCTPQGDWSPAAPTCEVIFCPSPPVIPNGRHTGKPLEVFPFG |
| | | KTVNYTCDPHPDRGTSFDLIGESTIRCTSDPQGNGVWSSPAPRCGILGHCQAP |
| | | DHFLFAKLKTQTNASDFPIGTSLKYECRPEYYGRPFSITCLDNLVWSSPKDVC |
| | | KRKSCKTPPDPVNGMVHVITDIQVGSRINYSCTTGHRLIGHSSAECILSGNAA |
| | | HWSTKPPICQRIPCGLPPTIANGDFISTNRENFHYGSVVTYRCNPGSGGRKVF |
| | | ELVGEPSIYCTSNDDQVGIWSGPAPQCHIPNKCTPPNVENGILVSDNRSLFSLN |
| | | EVVEFRCQPGFVMKGPRRVKCQALNKWEPELPSCSRVCQPPPDVLHAERTQ |
| | | RDKDNFSPGQEVFYSCEPGYDLRGAASMRCTPQGDWSPAAPTCEVIFCPSPP |
| | | VIPNGRHTGKPLEVFPFGKAVNYTCDPHPDRGTSFDLIGESTIRCTSDPQGNG |
| | | VWSSPAPRCGILGHCQAPDHFLFAKLKTQTNASDFPIGTSLKYECRPEYYGRP |
| | | FSITCLDNLVWSSPKDVCKRKSCKTPPDPVNGMVHVITDIQVGSRINYSCTTG |
| | | HRLIGHSSAECILSGNTAHWSTKPPICQRIPCGLPPTIANGDFISTNRENFHYGS |
| | | VVTYRCNLGSRGRKVFELVGEPSIYCTSNDDQVGIWSGPAPQCII |
| 21 | CR2CR1 | ISCGSPPPILNGRISYYSTPIAVGTVIRYSCSGTFRLIGEKSLLCITKDKVDGTWDKP |
| | #1 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFQCNAPEWLPFARPTNLTDEFEFP |
| | | IGTYLNYECRPGYSGRPFSIICLKNSVWTGAKDRCRRKSCRNPPDPVNGMVH |
| | | VIKGIQFGSQIKYSCTKGYRLIGSSSATCIISGDTVIWDNETPICDRIPCGLPPTI |
| | | TNGDFISTNRENFHYGSVVTYRCNPGSGGRKVFELVGEPSIYCTSNDDQVGIW |
| | | SGPAPQCIIPNKCTPPNVENGILVSDNRSLFSLNEVVEFRCQPGFVMKGPRRV |
| | | KCQALNKWEPELPSCSRVCQPPPDVLHAERTQRDKDNFSPGQEVFYSCEPGY |
| | | DLRGAASMRCTPQGDWSPAAPTCEVKSCDDFMGQLLNGRVLFPVNLQLGA |
| | | KVDFVCDEGFQLKGSSASYCVLAGMESLWNSSVPVCEQIFCPSPPVIPNGRHT |
| | | GKPLEVFPFGKTVNYTCDPHPDRGTSFDLIGESTIRCTSDPQGNGVWSSPAPR |
| | | CGILGHCQAPDHFLFAKLKTQTNASDFPIGTSLKYECRPEYYGRPFSITCLDN |
| | | LVWSSPKDVCKRKSCKTPPDPVNGMVHVITDIQVGSRINYSCTTGHRLIGHSS |
| | | AECILSGNAAHWSTKPPICQRIPCGLPPTIANGDFISTNRENFHYGSVVTYRCN |
| | | PGSGGRKVFELVGEPSIYCTSNDDQVGIWSGPAPQCII |
| 22 | CR2CR1 | ISCGSPPPILNGRISYYSTPIAVGTVIRYSCSGTFRLIGEKSLLCITKDKVDGTWDKP |
| | #1- | APKCEYFNKYSSCPEPIVPGGYKIRGSTPYRHGDSVTFACKTNFSMNGNKSVWCQ |
| | PNK | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFQCNAPEWLPFARPTNLTDEFEFP |
| | | IGTYLNYECRPGYSGRPFSIICLKNSVWTGAKDRCRRKSCRNPPDPVNGMVH |
| | | VIKGIQFGSQIKYSCTKGYRLIGSSSATCIISGDTVIWDNETPICDRIPCGLPPTI |
| | | TNGDFISTNRENFHYGSVVTYRCNPGSGGRKVFELVGEPSIYCTSNDDQVGIW |
| | | SGPAPQCIIPNKCTPPNVENGILVSDNRSLFSLNEVVEFRCQPGFVMKGPRRV |
| | | KCQALNKWEPELPSCSRVCQPPPDVLHAERTQRDKDNFSPGQEVFYSCEPGY |
| | | DLRGAASMRCTPQGDWSPAAPTCEVKSCDDFMGQLLNGRVLFPVNLQLGA |
| | | KVDFVCDEGFQLKGSSASYCVLAGMESLWNSSVPVCEQIFCPSPPVIPNGRHT |
| | | GKPLEVFPFGKTVNYTCDPHPDRGTSFDLIGESTIRCTSDPQGNGVWSSPAPR |
| | | CGILGHCQAPDHFLFAKLKTQTNASDFPIGTSLKYECRPEYYGRPFSITCLDN |
| | | LVWSSPKDVCKRKSCKTPPDPVNGMVHVITDIQVGSRINYSCTTGHRLIGHSS |
| | | AECILSGNAAHWSTKPPICQRIPCGLPPTIANGDFISTNRENFHYGSVVTYRCN |
| | | PGSGGRKVFELVGEPSIYCTSNDDQVGIWSGPAPQCHIPNK |
| 23 | CR2CR1 | ISCGSPPPILNGRISYYSTPIAVGTVIRYSCSGTFRLIGEKSLLCITKDKVDGTWDKP |
| | #2 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFGGGGSGGGGSQCNAPEWLPFARP |
| | | TNLTDEFEFPIGTYLNYECRPGYSGRPFSIICLKNSVWTGAKDRCRRKSCRNP |
| | | PDPVNGMVHVIKGIQFGSQIKYSCTKGYRLIGSSSATCIISGDTVIWDNETPIC |
| | | DRIPCGLPPTITNGDFISTNRENFHYGSVVTYRCNPGSGGRKVFELVGEPSIYC |
| | | TSNDDQVGIWSGPAPQCIIPNKCTPPNVENGILVSDNRSLFSLNEVVEFRCQPG |
| | | FVMKGPRRVKCQALNKWEPELPSCSRVCQPPPDVLHAERTQRDKDNFSPGQ |
| | | EVFYSCEPGYDLRGAASMRCTPQGDWSPAAPTCEVKSCDDFMGQLLNGRVL |
| | | FPVNLQLGAKVDFVCDEGFQLKGSSASYCVLAGMESLWNSSVPVCEQIFCPS |
| | | PPVIPNGRHTGKPLEVFPFGKTVNYTCDPHPDRGTSFDLIGESTIRCTSDPQGN |
| | | GVWSSPAPRCGILGHCQAPDHFLFAKLKTQTNASDFPIGTSLKYECRPEYYG |
| | | RPFSITCLDNLVWSSPKDVCKRKSCKTPPDPVNGMVHVITDIQVGSRINYSCT |
| | | TGHRLIGHSSAECILSGNAAHWSTKPPICQRIPCGLPPTIANGDFISTNRENFH |
| | | YGSVVTYRCNPGSGGRKVFELVGEPSIYCTSNDDQVGIWSGPAPQCIIPNK |
| 24 | CR2CR1 | ISCGSPPPILNGRISYYSTPIAVGTVIRYSCSGTFRLIGEKSLLCITKDKVDGTWDKP |
| | #3 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFQCNAPEWLPFARPTNLTDEFEFP |
| | | IGTYLNYECRPGYSGRPFSIICLKNSVWTGAKDRCRRKSCRNPPDPVNGMVH |
| | | VIKGIQFGSQIKYSCTKGYRLIGSSSATCIISGDTVIWDNETPICDRIPCGLPPTI |
| | | TNGDFISTNRENFHYGSVVTYRCNPGSGGRKVFELVGEPSIYCTSNDDQVGIW |
| | | SGPAPQCIIGHCQAPDHFLFAKLKTQTNASDFPIGTSLKYECRPEYYGRPFSIT |
| | | CLDNLVWSSPKDVCKRKSCKTPPDPVNGMVHVITDIQVGSRINYSCTTGHRLI |
| | | GHSSAECILSGNAAHWSTKPPICQRIPCGLPPTIANGDFISTNRENFHYGSVVT |
| | | YRCNPGSGGRKVFELVGEPSIYCTSNDDQVGIWSGPAPQCIIPNKGHCQAPDH |
| | | FLFAKLKTQTNASDFPIGTSLKYECRPEYYGRPFSITCLDNLVWSSPKDVCKR |
| | | KSCKTPPDPVNGMVHVITDIQVGSRINYSCTTGHRLIGHSSAECILSGNTAHW |
| | | STKPPICQRIPCGLPPTIANGDFISTNRENFHYGSVVTYRCNLGSRGRKVFELV |
| | | GEPSIYCTSNDDQVGIWSGPAPQCII |
| 25 | CR2CR1 | ISCGSPPPILNGRISYYSTPIAVGTVIRYSCSGTFRLIGEKSLLCITKDKVDGTWDKP |
| | #4 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFGGGGSGGGGSQCNAPEWLPFARP |
| | | TNLTDEFEFPIGTYLNYECRPGYSGRPFSIICLKNSVWTGAKDRCRRKSCRNP |
| | | PDPVNGMVHVIKGIQFGSQIKYSCTKGYRLIGSSSATCIISGDTVIWDNETPIC |
| | | DRIPCGLPPTITNGDFISTNRENFHYGSVVTYRCNPGSGGRKVFELVGEPSIYC |
| | | TSNDDQVGIWSGPAPQCIIGHCQAPDHFLFAKLKTQTNASDFPIGTSLKYECR |
| | | PEYYGRPFSITCLDNLVWSSPKDVCKRKSCKTPPDPVNGMVHVITDIQVGSRI |
| | | NYSCTTGHRLIGHSSAECILSGNAAHWSTKPPICQRIPCGLPPTIANGDFISTN |
| | | RENFHYGSVVTYRCNPGSGGRKVFELVGEPSIYCTSNDDQVGIWSGPAPQCII |
| | | PNKGHCQAPDHFLFAKLKTQTNASDFPIGTSLKYECRPEYYGRPFSITCLDNL |
| | | VWSSPKDVCKRKSCKTPPDPVNGMVHVITDIQVGSRINYSCTTGHRLIGHSSA |
| | | ECILSGNTAHWSTKPPICQRIPCGLPPTIANGDFISTNRENFHYGSVVTYRCNL |
| | | GSRGRKVFELVGEPSIYCTSNDDQVGIWSGPAPQCII |
| 26 | CR2CR1 | ISCGSPPPILNGRISYYSTPIAVGTVIRYSCSGTFRLIGEKSLLCITKDKVDGTWDKP |
| | #5 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFQCNAPEWLPFARPTNLTDEFEFP |
| | | IGTYLNYECRPGYSGRPFSIICLKNSVWTGAKDRCRRKSCRNPPDPVNGMVH |
| | | VIKGIQFGSQIKYSCTKGYRLIGSSSATCIISGDTVIWDNETPICDRIPCGLPPTI |
| | | TNGDFISTNRENFHYGSVVTYRCNPGSGGRKVFELVGEPSIYCTSNDDQVGIW |
| | | SGPAPQCIIPNKCTPPNVENGILVSDNRSLFSLNEVVEFRCQPGFVMKGPRRV |
| | | KCQALNKWEPELPSCSRVCQPPPDVLHAERTQRDKDNFSPGQEVFYSCEPGY |
| | | DLRGAASMRCTPQGDWSPAAPTCEVKSCDDFMGQLLNGRVLFPVNLQLGA |
| | | KVDFVCDEGFQLKGSSASYCVLAGMESLWNSSVPVCEQIFCPSPPVIPNGRHT |
| | | GKPLEVFPFGKTVNYTCDPHPDRGTSFDLIGESTIRCTSDPQGNGVWSSPAPR |
| | | CGILGHCQAPDHFLFAKLKTQTNASDFPIGTSLKYECRPEYYGRPFSITCLDN |
| | | LVWSSPKDVCKRKSCKTPPDPVNGMVHVITDIQVGSRINYSCTTGHRLIGHSS |
| | | AECILSGNAAHWSTKPPICQRIPCGLPPTIANGDFISTNRENFHYGSVVTYRCN |
| | | PGSGGRKVFELVGEPSIYCTSNDDQVGIWSGPAPQCIIPNKCTPPNVENGILVS |
| | | DNRSLFSLNEVVEFRCQPGFVMKGPRRVKCQALNKWEPELPSCSRVCQPPPD |
| | | VLHAERTQRDKDNFSPGQEVFYSCEPGYDLRGAASMRCTPQGDWSPAAPTC |
| | | EVKSCDDFMGQLLNGRVLFPVNLQLGAKVDFVCDEGFQLKGSSASYCVLAG |
| | | MESLWNSSVPVCEQIFCPSPPVIPNGRHTGKPLEVFPFGKAVNYTCDPHPDRG |
| | | TSFDLIGESTIRCTSDPQGNGVWSSPAPRCGI |
| 27 | CR2CR1 | ISCGSPPPILNGRISYYSTPIAVGTVIRYSCSGTFRLIGEKSLLCITKDKVDGTWDKP |
| | #6 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFGGGGSGGGGSQCNAPEWLPFARP |
| | | TNLTDEFEFPIGTYLNYECRPGYSGRPFSIICLKNSVWTGAKDRCRRKSCRNP |
| | | PDPVNGMVHVIKGIQFGSQIKYSCTKGYRLIGSSSATCIISGDTVIWDNETPIC |
| | | DRIPCGLPPTITNGDFISTNRENFHYGSVVTYRCNPGSGGRKVFELVGEPSIYC |
| | | TSNDDQVGIWSGPAPQCIIPNKCTPPNVENGILVSDNRSLFSLNEVVEFRCQPG |
| | | FVMKGPRRVKCQALNKWEPELPSCSRVCQPPPDVLHAERTQRDKDNFSPGQ |
| | | EVFYSCEPGYDLRGAASMRCTPQGDWSPAAPTCEVKSCDDFMGQLLNGRVL |
| | | FPVNLQLGAKVDFVCDEGFQLKGSSASYCVLAGMESLWNSSVPVCEQIFCPS |
| | | PPVIPNGRHTGKPLEVFPFGKTVNYTCDPHPDRGTSFDLIGESTIRCTSDPQGN |
| | | GVWSSPAPRCGILGHCQAPDHFLFAKLKTQTNASDFPIGTSLKYECRPEYYG |
| | | RPFSITCLDNLVWSSPKDVCKRKSCKTPPDPVNGMVHVITDIQVGSRINYSCT |
| | | TGHRLIGHSSAECILSGNAAHWSTKPPICQRIPCGLPPTIANGDFISTNRENFH |
| | | YGSVVTYRCNPGSGGRKVFELVGEPSIYCTSNDDQVGIWSGPAPQCIIPNKCT |
| | | PPNVENGILVSDNRSLFSLNEVVEFRCQPGFVMKGPRRVKCQALNKWEPELP |
| | | SCSRVCQPPPDVLHAERTQRDKDNFSPGQEVFYSCEPGYDLRGAASMRCTPQ |
| | | GDWSPAAPTCEVKSCDDFMGQLLNGRVLFPVNLQLGAKVDFVCDEGFQLK |
| | | GSSASYCVLAGMESLWNSSVPVCEQIFCPSPPVIPNGRHTGKPLEVFPFGKAV |
| | | NYTCDPHPDRGTSFDLIGESTIRCTSDPQGNGVWSSPAPRCGI |
| 28 | CR2CR1 | ISCGSPPPILNGRISYYSTPIAVGTVIRYSCSGTFRLIGEKSLLCITKDKVDGTWDKP |
| | #13 | APKCEYFNKYSSCPEPIVPGGYKIRGSTPYRHGDSVTFACKTNFSMNGNKSVWCQ |
| | | ANNMWGPTRLPTCVSVFPLECPALPMIHNGHHTSENVGSIAPGLSVTYSCESGYL |
| | | LVGEKIINCLSSGKWSAVPPTCEEARCKSLGRFPNGKVKEPPILRVGVTANFFCDE |
| | | GYRLQGPPSSRCVIAGQGVAWTKMPVCEEIFQCNAPEWLPFARPTNLTDEFEFP |
| | | IGTYLNYECRPGYSGRPFSIICLKNSVWTGAKDRCRRKSCRNPPDPVNGMVH |
| | | VIKGIQFGSQIKYSCTKGYRLIGSSSATCIISGDTVIWDNETPICDRIPCGLPPTI |
| | | TNGDFISTNRENFHYGSVVTYRCNPGSGGRKVFELVGEPSIYCTSNDDQVGIW |
| | | SGPAPQCIIPNKCTPPNVENGILVSDNRSLFSLNEVVEFRCQPGFVMKGPRRV |
| | | KCQALNKWEPELPSCSRVCQPPPDVLHAERTQRDKDNFSPGQEVFYSCEPGY |
| | | DLRGAASMRCTPQGDWSPAAPTCEVKSCDDFMGQLLNGRVLFPVNLQLGA |
| | | KVDFVCDEGFQLKGSSASYCVLAGMESLWNSSVPVCEQIFCPSPPVIPNGRHT |
| | | GKPLEVFPFGKTVNYTCDPHPDRGTSFDLIGESTIRCTSDPQGNGVWSSPAPR |
| | | CGILGHCQAPDHFLFAKLKTQTNASDFPIGTSLKYECRPEYYGRPFSITCLDN |
| | | LVWSSPKDVCKRKSCKTPPDPVNGMVHVITDIQVGSRINYSCTTGHRLIGHSS |
| | | AECILSGNAAHWSTKPPICQRIPCGLPPTIANGDFISTNRENFHYGSVVTYRCN |
| | | PGSGGRKVFELVGEPSIYCTSNDDQVGIWSGPAPQCHIPNKCTPPNVENGILVS |
| | | DNRSLFSLNEVVEFRCQPGFVMKGPRRVKCQALNKWEPELPSCSRVCQPPPD |
| | | VLHAERTQRDKDNFSPGQEVFYSCEPGYDLRGAASMRCTPQGDWSPAAPTC |
| | | EVKSCDDFMGQLLNGRVLFPVNLQLGAKVDFVCDEGFQLKGSSASYCVLAG |
| | | MESLWNSSVPVCEQIFCPSPPVIPNGRHTGKPLEVFPFGKAVNYTCDPHPDRG |
| | | TSFDLIGESTIRCTSDPQGNGVWSSPAPRCGILGHCQAPDHFLFAKLKTQTNA |
| | | SDFPIGTSLKYECRPEYYGRPFSITCLDNLVWSSPKDVCKRKSCKTPPDPVNG |
| | | MVHVITDIQVGSRINYSCTTGHRLIGHSSAECILSGNTAHWSTKPPICQRIPCG |
| | | LPPTIANGDFISTNRENFHYGSVVTYRCNLGSRGRKVFELVGEPSIYCTSNDD |
| | | QVGIWSGPAPQCII |
| 29 | CR2CR1 | ctagttattaatagtaatcaattacggggtcattagttcatagcccatatatggagttccgcgttacataacttacggtaaat |
| | #7A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggccggattagttattattctacccccattgctgttggtaccgtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggaaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttcaatgcaatgccccagaatggcttccatttgccaggcctaccaaccta |
| | | actgatgaatttgagtttcccattgggacatatctgaactatgaatgccgccctggttattccggaagaccgttttctatcatc |
| | | tgcctaaaaaactcagtctggactggtgctaaggacaggtgcagacgtaaatcatgtcgtaatcctccagatcctgtgaat |
| | | ggcatggtgcatgtgatcaaaggcatccagttcggatcccaaattaaatattcttgtactaaaggataccgactcattggtt |
| | | cctcgtctgccacatgcatcatctcaggtgatactgtcatttgggataatgaaacacctatttgtgacagaattccttgtggg |
| | | ctaccccccaccatcaccaatggagatttcattagcaccaacagagagaattttcactatggatcagtggtgacctaccgc |
| | | tgcaatcctggaagcggagggagaaaggtgtttgagcttgtgggtgagccctccatatactgcaccagcaatgacgatca |
| | | agtgggcatctggagcggccccgcccctcagtgcattatacctaacaaatgcacgcctccaaatgtggaaaatggaatat |
| | | tggtatctgacaacagaagcttattttccttaaatgaagttgtggagtttaggtgtcagcctggctttgtcatgaaaggaccc |
| | | cgccgtgtgaagtgccaggccctgaacaaatgggagccggagctaccaagctgctccaggatcttttgtccaagtcctcc |
| | | agttattcctaatgggagacacacaggaaaacctctggaagtctttccctttgggaaaacagtaaattacacatgcgaccc |
| | | ccacccagacagagggacgagcttcgacctcattggagagagcaccatccgctgcacaagtgaccctcaagggaatgg |
| | | ggtttggagcagccctgcccctcgctgtggaattctgggtcactgtcaagccccagatcattttctgtttgccaagttgaaaa |
| | | cccaaaccaatgcatctgactttcccattgggacatctttaaagtacgaatgccgtcctgagtactacgggaggccattctc |
| | | tatcacatgtctagataacctggtctggtcaagtcccaaagatgtctgtaaacgtaaatcatgtaaaactcctccagatcca |
| | | gtgaatggcatggtgcatgtgatcacagacatccaggttggatccagaatcaactattcttgtactacagggcaccgactc |
| | | attggtcactcatctgctgaatgtatcctctcgggcaatgctgcccattggagcacgaagccgccaatttgtcaacgaattc |
| | | cttgtgggctaccccccaccatcgccaatggagatttcattagcaccaacagagagaattttcactatggatcagtggtga |
| | | cctaccgctgcaatcctggaagcggagggagaaaggtgtttgagcttgtgggtgagccctccatatactgcaccagcaat |
| | | gacgatcaagtgggcatctggagcggcccggcccctcagtgcattatacctaacaaatgcacgcctccaaatgtggaaaa |
| | | tggaatattggtatctgacaacagaagcttattttccttaaatgaagttgtggagtttaggtgtcagcctggctttgtcatgaa |
| | | aggaccccgccgtgtgaagtgccaggccctgaacaaatgggagccggagctaccaagctgctccaggatcttttgtccaa |
| | | gtcctccagttattcctaatgggagacacacaggaaaacctctggaagtctttccctttggaaaagcagtaaattacacat |
| | | gcgacccccacccagacagagggacgagcttcgacctcattggagagagcaccatccgctgcacaagtgaccctcaag |
| | | ggaatggggtttggagcagccctgcccctcgctgtggaattctgggtcactgtcaagccccagatcattttctgtttgccaa |
| | | gttgaaaacccaaaccaatgcatctgactttcccattgggacatctttaaagtacgaatgccgtcctgagtactacgggag |
| | | gccattctctatcacatgtctagataacctggtctggtcaagtcccaaagatgtctgtaaacgtaaatcatgtaaaactcctc |
| | | cagatccagtgaatggcatggtgcatgtgatcacagacatccaggttggatccagaatcaactattcttgtactacagggc |
| | | accgactcattggtcactcatctgctgaatgtatcctctcaggcaatactgcccattggagcacgaagccgccaatttgtca |
| | | acgaattccttgtgggctacccccaaccatcgccaatggagatttcattagcaccaacagagagaattttcactatggatc |
| | | agtggtgacctaccgctgcaatcttggaagcagagggagaaaggtgtttgagcttgtgggtgagccctccatatactgcac |
| | | cagcaatgacgatcaagtgggcatctggagcggccccgcccctcagtgcattatatagcaattgaataaaagatctttatttt |
| | | cattagatctgtgtgttggttttttgtgtg |
| 30 | CR2CR1 | ctagttattaatagtaatcaattacggggtcattagttcatagcccatatatggagttccgcgttacataacttacggtaaat |
| | #7B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctcttgagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctagtggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttcaatgcaatgccccagaatggcttccatttgccaggcctaccaacctaa |
| | | ctgatgaatttgagtttcccattgggacatatctgaactatgaatgccgccctggttatagcggaagaccattttctatcatct |
| | | gcctaaaaaactcagtctggactggtgctaaggacaggtgcagaaggaaatcatgtaggaatcctccagatcctgtgaat |
| | | ggcatggtgcatgtgatcaaaggcatccagtttggatcccaaattaaatattcttgtactaaaggataccgcctcattggttc |
| | | ctcatctgccacatgcatcatctcaggtgatactgtcatttgggataatgaaacacctatttgtgacagaattccttgtgggc |
| | | taccccccaccatcaccaatggagatttcattagcaccaacagagagaattttcactatggatcagtggtgacctaccgct |
| | | gcaatcctggaagcggagggagaaaggtgtttgagcttgtgggtgagccctccatatactgcaccagcaatgatgatcaa |
| | | gtgggcatctggagcggccccgcccctcagtgcattatacctaacaaatgcactcctccaaatgtggaaaatggaatattg |
| | | gtatctgacaacagaagcttattttccttaaatgaagttgtggagtttaggtgtcagcctggctttgtcatgaaaggaccccg |
| | | cagggtgaagtgccaggccctgaacaaatgggagccagagctaccaagctgctccaggatcttttgtccaagtcctccag |
| | | ttattcctaatgggagacacacaggaaaacctctggaagtctttccctttgggaaaacagtaaattacacatgtgaccccc |
| | | acccagacagagggactagctttgacctcattggagagagcaccatccgctgcacaagtgaccctcaagggaatggggt |
| | | ttggagcagccctgcccctagatgtggaattctgggtcactgtcaagccccagatcattttctgtttgccaagttgaaaaccc |
| | | aaaccaatgcatctgactttcccattgggacatctttaaagtatgaatgccgccctgagtactatgggaggccattctctatc |
| | | acatgtctagataacctggtctggtcaagtcccaaagatgtctgtaaaaggaaatcatgtaaaactcctccagatccagtg |
| | | aatggcatggtgcatgtgatcacagacatccaggttggatccagaatcaactattcttgtactactgggcaccgcctcattg |
| | | gtcactcatctgctgaatgtatcctcagtggcaatgctgcccattggagcactaagccgccaatttgtcaaagaattccttgt |
| | | gggctaccccccaccattgccaatggagatttcattagcaccaacagagagaattttcactatggatcagtggtgacctac |
| | | cgctgcaatcctggaagcggagggagaaaggtgtttgagcttgtgggtgagccctccatatactgcaccagcaatgatga |
| | | tcaagtgggcatctggagcggcccagcccctcagtgcattatacctaacaaatgcactcctccaaatgtggaaaatggaa |
| | | tattggtatctgacaacagaagcttattttccttaaatgaagttgtggagtttaggtgtcagcctggctttgtcatgaaagga |
| | | ccccgcagggtgaagtgccaggccctgaacaaatgggagccagagctaccaagctgctccaggatcttttgtccaagtcc |
| | | tccagttattcctaatgggagacacacaggaaaacctctggaagtctttccctttggaaaagcagtaaattacacatgtga |
| | | cccccacccagacagagggactagctttgacctcattggagagagcaccatccgctgcacaagtgaccctcaagggaat |
| | | ggggtttggagcagccctgcccctagatgtggaattctgggtcactgtcaagccccagatcattttctgtttgccaagttgaa |
| | | aacccaaaccaatgcatctgactttcccattgggacatctttaaagtatgaatgccgccctgagtactatgggaggccattc |
| | | tctatcacatgtctagataacctggtctggtcaagtcccaaagatgtctgtaaaaggaaatcatgtaaaactcctccagatc |
| | | cagtgaatggcatggtgcatgtgatcacagacatccaggttggatccagaatcaactattcttgtactactgggcaccgcct |
| | | cattggtcactcatctgctgaatgtatcctctcaggcaatactgcccattggagcactaagccgccaatttgtcaaagaattc |
| | | cttgtgggctacccccaaccattgccaatggagatttcattagcaccaacagagagaattttcactatggatcagtggtgac |
| | | ctaccgctgcaatcttggaagcagagggagaaaggtgtttgagcttgtgggtgagccctccatatactgcaccagcaatg |
| | | atgatcaagtgggcatctggagcggccccgcccctcagtgcattatatagcaattgaataaaagatctttattttcattagatct |
| | | gtgtgttggttttttgtgtg |
| 31 | CR2CR1 | catagcccatatatggagttccgcgttacataacttacggtaaatggcccgcctggctgaccgcccaacgacccccgccca |
| | #8A | ttgacgtcaataatgacgtatgttcccatagtaacgccaatagggactttccattgacgtcaatgggtggactatttacggt |
| | | aaactgcccacttggcagtacatcaagtgtatcatatgccaagtacgccccctattgacgtcaatgacggtaaatggcccg |
| | | cctggcattatgcccagtacatgaccttatgggactttcctacttggcagtacatctacgtattagtcatcgctattaccatgg |
| | | tcgaggtgagccccacgttctgcttcactctccccatctcccccccctccccacccccaattttgtatttatttattttttaattat |
| | | tttgtgcagcgatgggggcggggggggggggggggcgcgcgccaggcggggcggggcggggcgaggggcggggcgg |
| | | ggcgaggcggagaggtgcggcggcagccaatcagagcggcgcgctccgaaagtttccttttatggcgaggcggcggcg |
| | | gcggcggccctataaaaagcgaagcgcgcggcgggcgggagtcgctgcgacgctgccttcgccccgtgccccgctccgc |
| | | cgccgcctcgcgccgcccgccccggctctgactgaccgcgttactcccacaggtgagcggggggacggcccttctcctcc |
| | | gggctgtaattagcgcttggtttaatgacggcttgtttcttttctgtggctgcgtgaaagccttgaggggctccgggagctag |
| | | agcctctgctaaccatgttcatgccttcttctttttcctacagctcctgggcaacgtgctggttattgtgctgtctcatcattttg |
| | | gcaaagcggccgcaagcttgaattcatttaaatccagctagcgccaccatgaggctcctggctttcctgagtctgctggcctt |
| | | ggtgctgcaggagacagggacaatttcttgtggctctcctccgcctatcctaaatggccggattagttattattctacccccattgct |
| | | gttggtaccgtgataaggtacagttgttcaggtaccttccgcctcattggagaaaaaagtctattatgcataactaaagacaaagtgg |
| | | atggaacctgggataaacctgctcctaaatgtgaatatttcaataaatattcttcttgccctgagcccatagtaccaggaggatacaa |
| | | aattagaggctctacaccctacagacatggtgattctgtgacatttgcctgtaaaaccaacttctccatgaacggaaacaagtctgttt |
| | | ggtgtcaagcaaataatatgtgggggccgacacgactaccaacctgtgtaagtgttttccctctcgagtgtccagcacttcctatgat |
| | | ccacaatggacatcacacaagtgagaatgttggctccattgctccaggattgtctgtgacttacagctgtgaatctggttacttgcttg |
| | | ttggagaaaagatcattaactgtttgtcttcgggaaaatggagtgctgtcccccccacatgtgaagaggcacgctgtaaatctctag |
| | | gacgatttcccaatgggaaggtaaaggagcctccaattctccgggttggtgtaactgcaaactttttctgtgatgaagggtatcgact |
| | | gcaaggcccaccttctagtcggtgtgtaattgctggacagggagttgcttggaccaaaatgccagtatgtgaagaaatttttggtgg |
| | | aggtggatcgggaggtggtggatctcaatgcaatgccccagaatggcttccatttgccaggcctaccaacctaactgatga |
| | | atttgagtttcccattgggacatatctgaactatgaatgccgccctggttattccggaagaccgttttctatcatctgcctaaa |
| | | aaactcagtctggactggtgctaaggacaggtgcagacgtaaatcatgtcgtaatcctccagatcctgtgaatggcatggt |
| | | gcatgtgatcaaaggcatccagttcggatcccaaattaaatattcttgtactaaaggataccgactcattggttcctcgtctg |
| | | ccacatgcatcatctcaggtgatactgtcatttgggataatgaaacacctatttgtgacagaattccttgtgggctacccccc |
| | | accatcaccaatggagatttcattagcaccaacagagagaattttcactatggatcagtggtgacctaccgctgcaatcct |
| | | ggaagcggagggagaaaggtgtttgagcttgtgggtgagccctccatatactgcaccagcaatgacgatcaagtgggca |
| | | tctggagcggccccgcccctcagtgcattatacctaacaaatgcacgcctccaaatgtggaaaatggaatattggtatctg |
| | | acaacagaagcttattttccttaaatgaagttgtggagtttaggtgtcagcctggctttgtcatgaaaggaccccgccgtgt |
| | | gaagtgccaggccctgaacaaatgggagccggagctaccaagctgctccaggatcttttgtccaagtcctccagttattcc |
| | | taatgggagacacacaggaaaacctctggaagtctttccctttgggaaaacagtaaattacacatgcgacccccacccag |
| | | acagagggacgagcttcgacctcattggagagagcaccatccgctgcacaagtgaccctcaagggaatggggtttggag |
| | | cagccctgcccctcgctgtggaattctgggtcactgtcaagccccagatcattttctgtttgccaagttgaaaacccaaacc |
| | | aatgcatctgactttcccattgggacatctttaaagtacgaatgccgtcctgagtactacgggaggccattctctatcacatg |
| | | tctagataacctggtctggtcaagtcccaaagatgtctgtaaacgtaaatcatgtaaaactcctccagatccagtgaatgg |
| | | catggtgcatgtgatcacagacatccaggttggatccagaatcaactattcttgtactacagggcaccgactcattggtcac |
| | | tcatctgctgaatgtatcctctcgggcaatgctgcccattggagcacgaagccgccaatttgtcaacgaattccttgtgggct |
| | | accccccaccatcgccaatggagatttcattagcaccaacagagagaattttcactatggatcagtggtgacctaccgctg |
| | | caatcctggaagcggagggagaaaggtgtttgagcttgtgggtgagccctccatatactgcaccagcaatgacgatcaag |
| | | tgggcatctggagcggcccggcccctcagtgcattatacctaacaaatgcacgcctccaaatgtggaaaatggaatattg |
| | | gtatctgacaacagaagcttattttccttaaatgaagttgtggagtttaggtgtcagcctggctttgtcatgaaaggaccccg |
| | | ccgtgtgaagtgccaggccctgaacaaatgggagccggagctaccaagctgctccaggatcttttgtccaagtcctccagt |
| | | tattcctaatgggagacacacaggaaaacctctggaagtctttccctttggaaaagcagtaaattacacatgcgaccccca |
| | | cccagacagagggacgagcttcgacctcattggagagagcaccatccgctgcacaagtgaccctcaagggaatggggtt |
| | | tggagcagccctgcccctcgctgtggaattctgggtcactgtcaagccccagatcattttctgtttgccaagttgaaaaccc |
| | | aaaccaatgcatctgactttcccattgggacatctttaaagtacgaatgccgtcctgagtactacgggaggccattctctat |
| | | cacatgtctagataacctggtctggtcaagtcccaaagatgtctgtaaacgtaaatcatgtaaaactcctccagatccagtg |
| | | aatggcatggtgcatgtgatcacagacatccaggttggatccagaatcaactattcttgtactacagggcaccgactcatt |
| | | ggtcactcatctgctgaatgtatcctctcaggcaatactgcccattggagcacgaagccgccaatttgtcaacgaattcctt |
| | | gtgggctacccccaaccatcgccaatggagatttcattagcaccaacagagagaattttcactatggatcagtggtgacct |
| | | accgctgcaatcttggaagcagagggagaaaggtgtttgagcttgtgggtgagccctccatatactgcaccagcaatgac |
| | | gatcaagtgggcatctggagcggccccgcccctcagtgcattatatagcaattgaataaaagatctttattttcattagatctgt |
| | | gtgttggttttttgtgtg |
| 32 | CR2CR1 | ctagttattaatagtaatcaattacggggtcattagttcatagcccatatatggagttccgcgttacataacttacggtaaat |
| | #8B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctcttgagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctagtggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttggtggaggtggatcaggaggtggtggatctcaatgcaatgccccagaat |
| | | ggcttccatttgccaggcctaccaacctaactgatgaatttgagtttcccattgggacatatctgaactatgaatgccgccct |
| | | ggttatagcggaagaccattttctatcatctgcctaaaaaactcagtctggactggtgctaaggacaggtgcagaaggaa |
| | | atcatgtaggaatcctccagatcctgtgaatggcatggtgcatgtgatcaaaggcatccagtttggatcccaaattaaatat |
| | | tcttgtactaaaggataccgcctcattggttccagttctgccacatgcatcatctcaggtgatactgtcatttgggataatga |
| | | aacacctatttgtgacagaattccttgtgggctaccccccaccatcaccaatggagatttcattagcaccaacagagagaa |
| | | ttttcactatggatcagtggtgacctaccgctgcaatcctggaagcggagggagaaaggtgtttgagcttgtgggtgagcc |
| | | ctccatatactgcaccagcaatgatgatcaagtgggcatctggagcggccccgcccctcagtgcattatacctaacaaatg |
| | | cactcctccaaatgtggaaaatggaatattggtatctgacaacagaagcttattttccttaaatgaagttgtggagtttaggt |
| | | gtcagcctggctttgtcatgaaaggaccccgcagggtgaagtgccaggccctgaacaaatgggagccagagctaccaag |
| | | ctgctccaggatcttttgtccaagtcctccagttattcctaatgggagacacacaggaaaacctctggaagtctttccctttg |
| | | ggaaaacagtaaattacacatgtgacccccacccagacagagggactagctttgacctcattggagagagcaccatccg |
| | | ctgcacaagtgaccctcaagggaatggggtttggagcagccctgcccctagatgtggaattctgggtcactgtcaagcccc |
| | | agatcattttctgtttgccaagttgaaaacccaaaccaatgcatctgactttcccattgggacatctttaaagtatgaatgcc |
| | | gccctgagtactatgggaggccattctctatcacatgtctagataacctggtctggtcaagtcccaaagatgtctgtaaaag |
| | | gaaatcatgtaaaactcctccagatccagtgaatggcatggtgcatgtgatcacagacatccaggttggatccagaatca |
| | | actattcttgtactactgggcaccgcctcattggtcactcatctgctgaatgtatcctctcaggcaatgctgcccattggagc |
| | | actaagccgccaatttgtcaaagaattccttgtgggctaccccccaccattgccaatggagatttcattagcaccaacaga |
| | | gagaattttcactatggatcagtggtgacctaccgctgcaatcctggaagcggagggagaaaggtgtttgagcttgtgggt |
| | | gagccctccatatactgcaccagcaatgatgatcaagtgggcatctggagcggcccagcccctcagtgcattatacctaac |
| | | aaatgcactcctccaaatgtggaaaatggaatattggtatctgacaacagaagcttattttccttaaatgaagttgtggagt |
| | | ttaggtgtcagcctggctttgtcatgaaaggaccccgcagggtgaagtgccaggccctgaacaaatgggagccagagct |
| | | accaagctgctccaggatcttttgtccaagtcctccagttattcctaatgggagacacacaggaaaacctctggaagtcttt |
| | | ccctttggaaaagcagtaaattacacatgtgacccccacccagacagagggactagctttgacctcattggagagagcac |
| | | catccgctgcacaagtgaccctcaagggaatggggtttggagcagccctgcccctagatgtggaattctgggtcactgtca |
| | | agccccagatcattttctgtttgccaagttgaaaacccaaaccaatgcatctgactttcccattgggacatctttaaagtatg |
| | | aatgccgccctgagtactatgggaggccattctctatcacatgtctagataacctggtctggtcaagtcccaaagatgtctg |
| | | taaaaggaaatcatgtaaaactcctccagatccagtgaatggcatggtgcatgtgatcacagacatccaggttggatcca |
| | | gaatcaactattcttgtactactgggcaccgcctcattggtcactcatctgctgaatgtatcctctcaggcaatactgcccatt |
| | | ggagcactaagccgccaatttgtcaaagaattccttgtgggctacccccaaccattgccaatggagatttcattagcacca |
| | | acagagagaattttcactatggatcagtggtgacctaccgctgcaatcttggaagcagagggagaaaggtgtttgagctt |
| | | gtgggtgagccctccatatactgcaccagcaatgatgatcaagtgggcatctggagcggccccgcccctcagtgcattata |
| | | tagcaattgaataaaagatctttattttcattagatctgtgtgttggttttttgtgtg |
| 33 | CR2CR1 | ctagttattaatagtaatcaattacggggtcattagttcatagcccatatatggagttccgcgttacataacttacggtaaat |
| | #8C | ggcccgcctggctgaccgcccaacgacccccgcccattgacgtcaataatgacgtatgttcccatagtaacgccaatagg |
| | | gactttccattgacgtcaatgggggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tctggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctcttgagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctagtggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttggtggaggtggatcaggaggtggtggatctcaatgcaatgccccagaat |
| | | ggcttccatttgccaggcctaccaacctaactgatgaatttgagtttcccattgggacatatctgaactatgaatgccgccct |
| | | ggttatagcggaagaccattttctatcatctgcctaaaaaactcagtctggactggtgctaaggacaggtgcagaaggaa |
| | | atcatgtaggaatcctccagatcctgtgaatggcatggtgcatgtgatcaaaggcatccagtttggatcccaaattaaatat |
| | | tcttgtactaaaggataccgcctcattggttccagttctgccacatgcatcatctcaggtgatactgtcatttgggataatga |
| | | aacacctatttgtgacagaattccttgtgggctaccccccaccatcaccaatggagatttcattagcaccaacagagagaa |
| | | ttttcactatggatcagtggtgacctaccgctgcaatcctggaagcggagggagaaaggtgtttgagcttgtgggtgagcc |
| | | ctccatatactgcaccagcaatgatgatcaagtgggcatctggagcggccccgcccctcagtgcattatacctaacaaatg |
| | | cactcctccaaatgtggaaaatggaatattggtatctgacaacagaagcttattttccttaaatgaagttgtggagtttaggt |
| | | gtcagcctggctttgtcatgaaaggaccccgcagggtgaagtgccaggccctgaacaaatgggagccagagctaccaag |
| | | ctgctccaggatcttttgtccaagtcctccagttattcctaatgggagacacacaggaaaacctctggaagtctttccctttg |
| | | ggaaaacagtaaattacacatgtgacccccacccagacagagggactagctttgacctcattggagagagcaccatccg |
| | | ctgcacaagtgaccctcaagggaatggggtttggagcagccctgcccctagatgtggaattctgggtcactgtcaagcccc |
| | | agatcattttctgtttgccaagttgaaaacccaaaccaatgcatctgactttcccattgggacatctttaaagtatgaatgcc |
| | | gccctgagtactatgggaggccattctctatcacatgtctagataacctggtctggtcaagtcccaaagatgtctgtaaaag |
| | | gaaatcatgtaaaactcctccagatccagtgaatggcatggtgcatgtgatcacagacatccaggttggatccagaatca |
| | | actattcttgtactactgggcaccgcctcattggtcactcatctgctgaatgtatcctctcaggcaatgctgcccattggagc |
| | | actaagccgccaatttgtcaaagaattccttgtgggctaccccccaccattgccaatggagatttcattagcaccaacaga |
| | | gagaattttcactatggatcagtggtgacctaccgctgcaatcctggaagcggagggagaaaggtgtttgagcttgtgggt |
| | | gagccctccatatactgcaccagcaatgatgatcaagtgggcatctggagcggcccagcccctcagtgcattatacctaac |
| | | aaatgcactcctccaaatgtggaaaatggaatattggtatctgacaacagaagcttattttccttaaatgaagttgtggagt |
| | | ttaggtgtcagcctggctttgtcatgaaaggaccccgcagggtgaagtgccaggccctgaacaaatgggagccagagct |
| | | accaagctgctccaggatcttttgtccaagtcctccagttattcctaatgggagacacacaggaaaacctctggaagtcttt |
| | | ccctttggaaaagcagtaaattacacatgtgacccccacccagacagagggactagctttgacctcattggagagagcac |
| | | catccgctgcacaagtgaccctcaagggaatggggtttggagcagccctgcccctagatgtggaattctgggtcactgtca |
| | | agccccagatcattttctgtttgccaagttgaaaacccaaaccaatgcatctgactttcccattgggacatctttaaagtatg |
| | | aatgccgccctgagtactatgggaggccattctctatcacatgtctagataacctggtctggtcaagtcccaaagatgtctg |
| | | taaaaggaaatcatgtaaaactcctccagatccagtgaatggcatggtgcatgtgatcacagacatccaggttggatcca |
| | | gaatcaactattcttgtactactgggcaccgcctcattggtcactcatctgctgaatgtatcctctcaggcaatactgcccatt |
| | | ggagcactaagccgccaatttgtcaaagaattccttgtgggctacccccaaccattgccaatggagatttcattagcacca |
| | | acagagagaattttcactatggatcagtggtgacctaccgctgcaatcttggaagcagagggagaaaggtgtttgagctt |
| | | gtgggtgagccctccatatactgcaccagcaatgatgatcaagtgggcatctggagcggccccgcccctcagtgcattata |
| | | tagcaattgctgtgccttctagttgccagccatctgttgtttgcccctcccccgtgccttccttgaccctggaaggtgccactcccac |
| | | tgtcctttcctaataaaatgaggaaattgcatcgcattgtctgagtaggtgtcattctattctggggggtggggggggcaggac |
| | | agcaagggggaggattgggaagacaatagcaggcatgctggggatgcggtgggctctatg |
| 34 | CR2CR1 | ctagttattaatagtaatcaattacggggtcattagttcatagcccatatatggagttccgcgttacataacttacggtaaat |
| | #9A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggccggattagttattattctacccccattgctgttggtaccgtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttggtggaggtggatcgggaggtggtggatctcaatgcaatgccccagaa |
| | | tggcttccatttgccaggcctaccaacctaactgatgaatttgagtttcccattgggacatatctgaactatgaatgccgccc |
| | | tggttattccggaagaccgttttctatcatctgcctaaaaaactcagtctggactggtgctaaggacaggtgcagacgtaaa |
| | | tcatgtcgtaatcctccagatcctgtgaatggcatggtgcatgtgatcaaaggcatccagttcggatcccaaattaaatatt |
| | | cttgtactaaaggataccgactcattggttcctcgtctgccacatgcatcatctcaggtgatactgtcatttgggataatgaa |
| | | acacctatttgtgacagaattccttgtgggctaccccccaccatcaccaatggagatttcattagcaccaacagagagaatt |
| | | ttcactatggatcagtggtgacctaccgctgcaatcctggaagcggagggagaaaggtgtttgagcttgtgggtgagccct |
| | | ccatatactgcaccagcaatgacgatcaagtgggcatctggagcggccccgcccctcagtgcattatacctaacaaatgc |
| | | acgcctccaaatgtggaaaatggaatattggtatctgacaacagaagcttattttccttaaatgaagttgtggagtttaggt |
| | | gtcagcctggctttgtcatgaaaggaccccgccgtgtgaagtgccaggccctgaacaaatgggagccggagctaccaag |
| | | ctgctccaggggtggaggtggatcgggaggtggtggatctatcttttgtccaagtcctccagttattcctaatgggagacaca |
| | | caggaaaacctctggaagtctttccctttgggaaaacagtaaattacacatgcgacccccacccagacagagggacgag |
| | | cttcgacctcattggagagagcaccatccgctgcacaagtgaccctcaagggaatggggtttggagcagccctgcccctc |
| | | gctgtggaattctgggtcactgtcaagccccagatcattttctgtttgccaagttgaaaacccaaaccaatgcatctgacttt |
| | | cccattgggacatctttaaagtacgaatgccgtcctgagtactacgggaggccattctctatcacatgtctagataacctgg |
| | | tctggtcaagtcccaaagatgtctgtaaacgtaaatcatgtaaaactcctccagatccagtgaatggcatggtgcatgtgat |
| | | cacagacatccaggttggatccagaatcaactattcttgtactacagggcaccgactcattggtcactcatctgctgaatgt |
| | | atcctctcgggcaatgctgcccattggagcacgaagccgccaatttgtcaacgaattccttgtgggctaccccccaccatcg |
| | | ccaatggagatttcattagcaccaacagagagaattttcactatggatcagtggtgacctaccgctgcaatcctggaagcg |
| | | gagggagaaaggtgtttgagcttgtgggtgagccctccatatactgcaccagcaatgacgatcaagtgggcatctggagc |
| | | ggcccggcccctcagtgcattatacctaacaaatgcacgcctccaaatgtggaaaatggaatattggtatctgacaacag |
| | | aagcttattttccttaaatgaagttgtggagtttaggtgtcagcctggctttgtcatgaaaggaccccgccgtgtgaagtgcc |
| | | aggccctgaacaaatgggagccggagctaccaagctgctccaggggtggaggtggatcgggaggtggtggatctatctttt |
| | | gtccaagtcctccagttattcctaatgggagacacacaggaaaacctctggaagtctttccctttggaaaagcagtaaatt |
| | | acacatgcgacccccacccagacagagggacgagcttcgacctcattggagagagcaccatccgctgcacaagtgaccc |
| | | tcaagggaatggggtttggagcagccctgcccctcgctgtggaattctgggtcactgtcaagccccagatcattttctgtttg |
| | | ccaagttgaaaacccaaaccaatgcatctgactttcccattgggacatctttaaagtacgaatgccgtcctgagtactacg |
| | | ggaggccattctctatcacatgtctagataacctggtctggtcaagtcccaaagatgtctgtaaacgtaaatcatgtaaaac |
| | | tcctccagatccagtgaatggcatggtgcatgtgatcacagacatccaggttggatccagaatcaactattcttgtactaca |
| | | gggcaccgactcattggtcactcatctgctgaatgtatcctctcaggcaatactgcccattggagcacgaagccgccaattt |
| | | gtcaacgaattccttgtgggctacccccaaccatcgccaatggagatttcattagcaccaacagagagaattttcactatg |
| | | gatcagtggtgacctaccgctgcaatcttggaagcagagggagaaaggtgtttgagcttgtgggtgagccctccatatact |
| | | gcaccagcaatgacgatcaagtgggcatctggagcggccccgcccctcagtgcattatatagcaattgaataaaagatcttt |
| | | attttcattagatctgtgtgttggttttttgtgtg |
| 35 | CR2CR1 | ctagttattaatagtaatcaattacggggtcattagttcatagcccatatatggagttccgcgttacataacttacggtaaat |
| | #9B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctcttgagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctagtggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttggtggaggtggatcaggaggtggtggatctcaatgcaatgccccagaat |
| | | ggcttccatttgccaggcctaccaacctaactgatgaatttgagtttcccattgggacatatctgaactatgaatgccgccct |
| | | ggttatagcggaagaccattttctatcatctgcctaaaaaactcagtctggactggtgctaaggacaggtgcagaaggaa |
| | | atcatgtaggaatcctccagatcctgtgaatggcatggtgcatgtgatcaaaggcatccagtttggatcccaaattaaatat |
| | | tcttgtactaaaggataccgcctcattggttccagttctgccacatgcatcatctcaggtgatactgtcatttgggataatga |
| | | aacacctatttgtgacagaattccttgtgggctaccccccaccatcaccaatggagatttcattagcaccaacagagagaa |
| | | ttttcactatggatcagtggtgacctaccgctgcaatcctggaagcggagggagaaaggtgtttgagcttgtgggtgagcc |
| | | ctccatatactgcaccagcaatgatgatcaagtgggcatctggagcggccccgcccctcagtgcattatacctaacaaatg |
| | | cactcctccaaatgtggaaaatggaatattggtatctgacaacagaagcttattttccttaaatgaagttgtggagtttaggt |
| | | gtcagcctggctttgtcatgaaaggaccccgcagggtgaagtgccaggccctgaacaaatgggagccagagctaccaag |
| | | ctgctccaggggtggaggtggatcaggaggtgtggatctatcttttgtccaagtcctccagttattcctaatgggagacacag |
| | | caggaaaacctctggaagtctttccctttgggaaaacagtaaattacacatgtgacccccacccagacagagggactagc |
| | | tttgacctcattggagagagcaccatccgctgcacaagtgaccctcaagggaatggggtttggagcagccctgcccctag |
| | | atgtggaattctgggtcactgtcaagccccagatcattttctgtttgccaagttgaaaacccaaaccaatgcatctgactttc |
| | | ccattgggacatctttaaagtatgaatgccgccctgagtactatgggaggccattctctatcacatgtctagataacctggtc |
| | | tggtcaagtcccaaagatgtctgtaaaaggaaatcatgtaaaactcctccagatccagtgaatggcatggtgcatgtgatc |
| | | acagacatccaggttggatccagaatcaactattcttgtactactgggcaccgcctcattggtcactcatctgctgaatgtat |
| | | cctcagtggcaatgctgcccattggagcactaagccgccaatttgtcaaagaattccttgtgggctaccccccaccattgcc |
| | | aatggagatttcattagcaccaacagagagaattttcactatggatcagtggtgacctaccgctgcaatcctggaagcgga |
| | | gggagaaaggtgtttgagcttgtgggtgagccctccatatactgcaccagcaatgatgatcaagtgggcatctggagcgg |
| | | cccagcccctcagtgcattatacctaacaaatgcactcctccaaatgtggaaaatggaatattggtatctgacaacagaag |
| | | cttattttccttaaatgaagttgtggagtttaggtgtcagcctggctttgtcatgaaaggaccccgcagggtgaagtgccag |
| | | gccctgaacaaatgggagccagagctaccaagctgctccaggggtggaggtggatcaggaggtggtggatctatcttttgt |
| | | ccaagtcctccagttattcctaatgggagacacacaggaaaacctctggaagtctttccctttggaaaagcagtaaattac |
| | | acatgtgacccccacccagacagagggactagctttgacctcattggagagagcaccatccgctgcacaagtgaccctca |
| | | agggaatggggtttggagcagccctgcccctagatgtggaattctgggtcactgtcaagccccagatcattttctgtttgcc |
| | | aagttgaaaacccaaaccaatgcatctgactttcccattgggacatctttaaagtatgaatgccgccctgagtactatggg |
| | | aggccattctctatcacatgtctagataacctggtctggtcaagtcccaaagatgtctgtaaaaggaaatcatgtaaaactc |
| | | ctccagatccagtgaatggcatggtgcatgtgatcacagacatccaggttggatccagaatcaactattcttgtactactgg |
| | | gcaccgcctcattggtcactcatctgctgaatgtatcctctcaggcaatactgcccattggagcactaagccgccaatttgtc |
| | | aaagaattccttgtgggctacccccaaccattgccaatggagatttcattagcaccaacagagagaattttcactatggatc |
| | | agtggtgacctaccgctgcaatcttggaagcagagggagaaaggtgtttgagcttgtgggtgagccctccatatactgcac |
| | | cagcaatgatgatcaagtgggcatctggagcggccccgcccctcagtgcattatatagcaattgaataaaagatctttattt |
| | | tcattagatctgtgtgttggttttttgtgtg |
| 36 | CR2CR1 | ctagttattaatagtaatcaattacggggtcattagttcatagcccatatatggagttccgcgttacataacttacggtaaat |
| | #10A | ggcccgcctggctgaccgcccaacgacccccgcccattgacgtcaataatgacgtatgttcccatagtaacgccaatagg |
| | | gactttccattgacgtcaatgggggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggccggattagttattattctacccccattgctgttggtaccgtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttcaatgcaatgccccagaatggcttccatttgccaggcctaccaaccta |
| | | actgatgaatttgagtttcccattgggacatatctgaactatgaatgccgccctggttattccggaagaccgttttctatcatc |
| | | tgcctaaaaaactcagtctggactggtgctaaggacaggtgcagacgtaaatcatgtcgtaatcctccagatcctgtgaat |
| | | ggcatggtgcatgtgatcaaaggcatccagttcggatcccaaattaaatattcttgtactaaaggataccgactcattggtt |
| | | cctcgtctgccacatgcatcatctcaggtgatactgtcatttgggataatgaaacacctatttgtgacagaattccttgtggg |
| | | ctaccccccaccatcaccaatggagatttcattagcaccaacagagagaattttcactatggatcagtggtgacctaccgc |
| | | tgcaatcctggaagcggagggagaaaggtgtttgagcttgtgggtgagccctccatatactgcaccagcaatgacgatca |
| | | agtgggcatctggagcggccccgcccctcagtgcattatacctggtggaggtggatcgggaggtggtggatctggtggagg |
| | | tggatcgggaggtggtggatctggtggaggtggatcgggaggtggtggatctggtggaggtggatcgggaggtggtggatct |
| | | ggtggaggtggatcgggaggtggtggatctggtggaggtggatcgggaggtggtggatctatcttttgtccaagtcctccagt |
| | | tattcctaatgggagacacacaggaaaacctctggaagtctttccctttgggaaaacagtaaattacacatgcgaccccca |
| | | cccagacagagggacgagcttcgacctcattggagagagcaccatccgctgcacaagtgaccctcaagggaatggggtt |
| | | tggagcagccctgcccctcgctgtggaattctgggtcactgtcaagccccagatcattttctgtttgccaagttgaaaaccc |
| | | aaaccaatgcatctgactttcccattgggacatctttaaagtacgaatgccgtcctgagtactacgggaggccattctctat |
| | | cacatgtctagataacctggtctggtcaagtcccaaagatgtctgtaaacgtaaatcatgtaaaactcctccagatccagtg |
| | | aatggcatggtgcatgtgatcacagacatccaggttggatccagaatcaactattcttgtactacagggcaccgactcatt |
| | | ggtcactcatctgctgaatgtatcctctcgggcaatgctgcccattggagcacgaagccgccaatttgtcaacgaattcctt |
| | | gtgggctaccccccaccatcgccaatggagatttcattagcaccaacagagagaattttcactatggatcagtggtgacct |
| | | accgctgcaatcctggaagcggagggagaaaggtgtttgagcttgtgggtgagccctccatatactgcaccagcaatgac |
| | | gatcaagtgggcatctggagcggcccggcccctcagtgcattatacctggtggaggtggatcgggaggtggtggatctggt |
| | | ggaggtggatcgggaggtggtggatctggtggaggtggatcgggaggtggtggatctggtggaggtggatcgggaggtggt |
| | | ggatctggtggaggtggatcgggaggtggtggatctggtggaggtggatcgggaggtggtggatctatcttttgtccaagtcc |
| | | tccagttattcctaatgggagacacacaggaaaacctctggaagtctttccctttggaaaagcagtaaattacacatgcga |
| | | cccccacccagacagagggacgagcttcgacctcattggagagagcaccatccgctgcacaagtgaccctcaagggaat |
| | | ggggtttggagcagccctgcccctcgctgtggaattctgggtcactgtcaagccccagatcattttctgtttgccaagttgaa |
| | | aacccaaaccaatgcatctgactttcccattgggacatctttaaagtacgaatgccgtcctgagtactacgggaggccattc |
| | | tctatcacatgtctagataacctggtctggtcaagtcccaaagatgtctgtaaacgtaaatcatgtaaaactcctccagatc |
| | | cagtgaatggcatggtgcatgtgatcacagacatccaggttggatccagaatcaactattcttgtactacagggcaccgac |
| | | tcattggtcactcatctgctgaatgtatcctctcaggcaatactgcccattggagcacgaagccgccaatttgtcaacgaatt |
| | | ccttgtgggctacccccaaccatcgccaatggagatttcattagcaccaacagagagaattttcactatggatcagtggtg |
| | | acctaccgctgcaatcttggaagcagagggagaaaggtgtttgagcttgtgggtgagccctccatatactgcaccagcaa |
| | | tgacgatcaagtgggcatctggagcggccccgcccctcagtgcattatatagcaattgaataaaagatctttattttcattaga |
| | | tctgtgtgttggttttttgtgtg |
| 37 | CR2CR1 | ctagttattaatagtaatcaattacggggtcattagttcatagcccatatatggagttccgcgttacataacttacggtaaat |
| | #10B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggggg |
| | | agtcgctgcgacgctgccttcgccccgtgccccgctccgccgccgcctcgcgccgcccgccccggctctgactgaccgcgt |
| | | tactcccacaggtgagcggggggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggccggattagttattattctacccccattgctgttggtaccgtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttcaatgcaatgccccagaatggcttccatttgccaggcctaccaaccta |
| | | actgatgaatttgagtttcccattgggacatatctgaactatgaatgccgccctggttattccggaagaccgttttctatcatc |
| | | tgcctaaaaaactcagtctggactggtgctaaggacaggtgcagacgtaaatcatgtcgtaatcctccagatcctgtgaat |
| | | ggcatggtgcatgtgatcaaaggcatccagttcggatcccaaattaaatattcttgtactaaaggataccgactcattggtt |
| | | cctcgtctgccacatgcatcatctcaggtgatactgtcatttgggataatgaaacacctatttgtgacagaattccttgtggg |
| | | ctaccccccaccatcaccaatggagatttcattagcaccaacagagagaattttcactatggatcagtggtgacctaccgc |
| | | tgcaatcctggaagcggagggagaaaggtgtttgagcttgtgggtgagccctccatatactgcaccagcaatgacgatca |
| | | agtgggcatctggagcggccccgcccctcagtgcattatacctggtggaggtggatcgggaggtggtggatctggtggagg |
| | | tggatcgggaggtggtggatctggtggaggtggatcgatcttttgtccaagtcctccagttattcctaatgggagacacacag |
| | | gaaaacctctggaagtctttccctttgggaaaacagtaaattacacatgcgacccccacccagacagagggacgagcttc |
| | | gacctcattggagagagcaccatccgctgcacaagtgaccctcaagggaatggggtttggagcagccctgcccctcgctg |
| | | tggaattctgggtcactgtcaagccccagatcattttctgtttgccaagttgaaaacccaaaccaatgcatctgactttccca |
| | | ttgggacatctttaaagtacgaatgccgtcctgagtactacgggaggccattctctatcacatgtctagataacctggtctg |
| | | gtcaagtcccaaagatgtctgtaaacgtaaatcatgtaaaactcctccagatccagtgaatggcatggtgcatgtgatcac |
| | | agacatccaggttggatccagaatcaactattcttgtactacagggcaccgactcattggtcactcatctgctgaatgtatc |
| | | ctctcgggcaatgctgcccattggagcacgaagccgccaatttgtcaacgaattccttgtgggctaccccccaccatcgcc |
| | | aatggagatttcattagcaccaacagagagaattttcactatggatcagtggtgacctaccgctgcaatcctggaagcgga |
| | | gggagaaaggtgtttgagcttgtgggtgagccctccatatactgcaccagcaatgacgatcaagtgggcatctggagcgg |
| | | cccggcccctcagtgcattatacctggtggaggtggatcgggaggtggtggatctggtggaggtggatcgggaggtggtgg |
| | | atctggtggaggtcgatcgatcttttgtccaagtcctccagttattcctaatgggagacacacaggaaaacctctggaagtct |
| | | ttccctttggaaaagcagtaaattacacatgcgacccccacccagacagagggacgagcttcgacctcattggagagagc |
| | | accatccgctgcacaagtgaccctcaagggaatggggtttggagcagccctgcccctcgctgtggaattctgggtcactgt |
| | | caagccccagatcattttctgtttgccaagttgaaaacccaaaccaatgcatctgactttcccattgggacatctttaaagta |
| | | cgaatgccgtcctgagtactacgggaggccattctctatcacatgtctagataacctggtctggtcaagtcccaaagatgtc |
| | | tgtaaacgtaaatcatgtaaaactcctccagatccagtgaatggcatggtgcatgtgatcacagacatccaggttggatcc |
| | | agaatcaactattcttgtactacagggcaccgactcattggtcactcatctgctgaatgtatcctctcaggcaatactgccca |
| | | ttggagcacgaagccgccaatttgtcaacgaattccttgtgggctacccccaaccatcgccaatggagatttcattagcac |
| | | caacagagagaattttcactatggatcagtggtgacctaccgctgcaatcttggaagcagagggagaaaggtgtttgagc |
| | | ttgtgggtgagccctccatatactgcaccagcaatgacgatcaagtgggcatctggagcggccccgcccctcagtgcatta |
| | | tatagcaattgaataaaagatctttattttcattagatctgtgtgttggttttttgtgtg |
| 38 | CR2CR1 | ctagttattaatagtaatcaattacggggtcattagttcatagcccatatatggagttccgcgttacataacttacggtaaat |
| | #10C | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcggggcgg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctagtggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttcaatgcaatgccccagaatggcttccatttgccaggcctaccaacctaa |
| | | ctgatgaatttgagtttcccattgggacatatctgaactatgaatgccgccctggttatagcggaagaccattttctatcatct |
| | | gcctaaaaaactcagtctggactggtgctaaggacaggtgcagaaggaaatcatgtaggaatcctccagatcctgtgaat |
| | | ggcatggtgcatgtgatcaaaggcatccagtttggatcccaaattaaatattcttgtactaaaggataccgcctcattggttc |
| | | ctcatctgccacatgcatcatctcaggtgatactgtcatttgggataatgaaacacctatttgtgacagaattccttgtgggc |
| | | taccccccaccatcaccaatggagatttcattagcaccaacagagagaattttcactatggatcagtggtgacctaccgct |
| | | gcaatcctggaagcggagggagaaaggtgtttgagcttgtgggtgagccctccatatactgcaccagcaatgatgatcaa |
| | | gtgggcatctggagcggccccgcccctcagtgcattatacctggtggaggtggaagtggaggtggtggatctggtggaggt |
| | | ggatcaggaggtggtggatctggtggaggtggaagtggaggtggtggatctggtggaggtggatcaggaggtggtggatct |
| | | ggtggaggtggaagtggaggtggtggatctggtggaggtggatcaggaggtggtggatctatcttttgtccaagtcctccagt |
| | | tattcctaatgggagacacacaggaaaacctctggaagtctttccctttgggaaaacagtaaattacacatgtgaccccca |
| | | cccagacagagggactagctttgacctcattggagagagcaccatccgctgcacaagtgaccctcaagggaatggggttt |
| | | ggagcagccctgcccctaggtgtggaattctgggtcactgtcaagccccagatcattttctgtttgccaagttgaaaaccca |
| | | aaccaatgcatctgactttcccattgggacatctttaaagtatgaatgccgccctgagtactatgggaggccattctctatca |
| | | catgtctagataacctggtctggtcaagtcccaaagatgtctgtaaaaggaaatcatgtaaaactcctccagatccagtga |
| | | atggcatggtgcatgtgatcacagacatccaggttggatccagaatcaactattcttgtactactgggcaccgcctcattgg |
| | | tcactcatctgctgaatgtatcctcagtggcaatgctgcccattggagcactaagccgccaatttgtcaaaggattccttgtg |
| | | ggctaccccccaccattgccaatggagatttcattagcaccaacagagagaattttcactatggatcagtggtgacctacc |
| | | gctgcaatcctggaagcggagggagaaaggtgtttgagcttgtgggtgagccctccatatactgcaccagcaatgatgat |
| | | caagtgggcatctggagcggcccagcccctcagtgcattatacctggtggaggtggatcaggagctgctcgatctggtgga |
| | | ggtggaagtggaggtggtggatctggtggaggtggatcaggaggtggtggatctggtggaggtggaagtggaggtggtgga |
| | | tctggtggaggtggatcaggaggtggtggatctggtggaggtggaagtggaggtggtggatctatcttttgtccaagtcctcc |
| | | agttattcctaatgggagacacacaggaaaacctctggaagtctttccctttggaaaagcagtaaattacacatgtgaccc |
| | | ccacccagacagagggactagctttgacctcattggagagagcaccatccgctgcacaagtgaccctcaagggaatggg |
| | | gtttggagcagccctgcccctagatgtggaattctgggtcactgtcaagccccagatcattttctgtttgccaagttgaaaac |
| | | ccaaaccaatgcatctgactttcccattgggacatctttaaagtatgaatgccgccctgagtactatgggaggccattctct |
| | | atcacatgtctagataacctggtctggtcaagtcccaaagatgtctgtaaaaggaaatcatgtaaaactcctccagatcca |
| | | gtgaatggcatggtgcatgtgatcacagacatccaggttggatccagaatcaactattcttgtactactgggcaccgcctca |
| | | ttggtcactcatctgctgaatgtatcctctcaggcaatactgcccattggagcactaagccgccaatttgtcaaagaattcct |
| | | tgtgggctacccccaaccattgccaatggagatttcattagcaccaacagagagaattttcactatggatcagtggtgacct |
| | | accgctgcaatcttggaagcagagggagaaaggtgtttgagcttgtgggtgagccctccatatactgcaccagcaatgat |
| | | gatcaagtgggcatctggagcggccccgcccctcagtgcattatatagcaattgaataaaagatctttattttcattagatctgt |
| | | gtgttggttttttgtgtg |
| 39 | CR2CR1 | ctagttattaatagtaatcaattacggggtcattagttcatagcccatatatggagttccgcgttacataacttacggtaaat |
| | #10D | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctagtggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttcaatgcaatgccccagaatggcttccatttgccaggcctaccaacctaa |
| | | ctgatgaatttgagtttcccattgggacatatctgaactatgaatgccgccctggttatagcggaagaccattttctatcatct |
| | | gcctaaaaaactcagtctggactggtgctaaggacaggtgcagaaggaaatcatgtaggaatcctccagatcctgtgaat |
| | | ggcatggtgcatgtgatcaaaggcatccagtttggatcccaaattaaatattcttgtactaaaggataccgcctcattggttc |
| | | ctcatctgccacatgcatcatctcaggtgatactgtcatttgggataatgaaacacctatttgtgacagaattccttgtgggc |
| | | taccccccaccatcaccaatggagatttcattagcaccaacagagagaattttcactatggatcagtggtgacctaccgct |
| | | gcaatcctggaagcggagggagaaaggtgtttgagcttgtgggtgagccctccatatactgcaccagcaatgatgatcaa |
| | | gtgggcatctggagcggccccgcccctcagtgcattatacctggtggaggtggaagtggaggtggtggatctggtggaggt |
| | | ggatcaggaggtggtggatctggtggaggtggaagtatcttttgtccaagtcctccagttattcctaatgggagacacacag |
| | | gaaaacctctggaagtctttccctttgggaaaacagtaaattacacatgtgacccccacccagacagagggactagctttg |
| | | acctcattggagagagcaccatccgctgcacaagtgaccctcaagggaatggggtttggagcagccctgcccctaggtgt |
| | | ggaattctgggtcactgtcaagccccagatcattttctgtttgccaagttgaaaacccaaaccaatgcatctgactttcccat |
| | | tgggacatctttaaagtatgaatgccgccctgagtactatgggaggccattctctatcacatgtctagataacctggtctggt |
| | | caagtcccaaagatgtctgtaaaaggaaatcatgtaaaactcctccagatccagtgaatggcatggtgcatgtgatcaca |
| | | gacatccaggttggatccagaatcaactattcttgtactactgggcaccgcctcattggtcactcatctgctgaatgtatcct |
| | | cagtggcaatgctgcccattggagcactaagccgccaatttgtcaaaggattccttgtgggctaccccccaccattgccaat |
| | | ggagatttcattagcaccaacagagagaattttcactatggatcagtggtgacctaccgctgcaatcctggaagcggagg |
| | | gagaaaggtgtttgagcttgtgggtgagccctccatatactgcaccagcaatgatgatcaagtgggcatctggagcggcc |
| | | cagcccctcagtgcattatacctggtggaggtggatcaggaggtgtggatctggtggaggtggaagtggaggtggtggat |
| | | ctggtggaggtggatcaatcttttgtccaagtcctccagttattcctaatgggagacacacaggaaaacctctggaagtcttt |
| | | ccctttggaaaagcagtaaattacacatgtgacccccacccagacagagggactagctttgacctcattggagagagcac |
| | | catccgctgcacaagtgaccctcaagggaatggggtttggagcagccctgcccctagatgtggaattctgggtcactgtca |
| | | agccccagatcattttctgtttgccaagttgaaaacccaaaccaatgcatctgactttcccattgggacatctttaaagtatg |
| | | aatgccgccctgagtactatgggaggccattctctatcacatgtctagataacctggtctggtcaagtcccaaagatgtctg |
| | | taaaaggaaatcatgtaaaactcctccagatccagtgaatggcatggtgcatgtgatcacagacatccaggttggatcca |
| | | gaatcaactattcttgtactactgggcaccgcctcattggtcactcatctgctgaatgtatcctctcaggcaatactgcccatt |
| | | ggagcactaagccgccaatttgtcaaagaattccttgtgggctacccccaaccattgccaatggagatttcattagcacca |
| | | acagagagaattttcactatggatcagtggtgacctaccgctgcaatcttggaagcagagggagaaaggtgtttgagctt |
| | | gtgggtgagccctccatatactgcaccagcaatgatgatcaagtgggcatctggagcggccccgcccctcagtgcattata |
| | | tagcaattgaataaaagatctttattttcattagatctgtgtgttggttttttgtgtg |
| 40 | CR2CR1 | ctagttattaatagtaatcaattacggggtcattagttcatagcccatatatggagttccgcgttacataacttacggtaaat |
| | #11A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggccggattagttattattctacccccattgctgttggtaccgtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttggtggaggtggatcgggaggtggtggatctcaatgcaatgccccagaa |
| | | tggcttccatttgccaggcctaccaacctaactgatgaatttgagtttcccattgggacatatctgaactatgaatgccgccc |
| | | tggttattccggaagaccgttttctatcatctgcctaaaaaactcagtctggactggtgctaaggacaggtgcagacgtaaa |
| | | tcatgtcgtaatcctccagatcctgtgaatggcatggtgcatgtgatcaaaggcatccagttcggatcccaaattaaatatt |
| | | cttgtactaaaggataccgactcattggttcctcgtctgccacatgcatcatctcaggtgatactgtcatttgggataatgaa |
| | | acacctatttgtgacagaattccttgtgggctaccccccaccatcaccaatggagatttcattagcaccaacagagagaatt |
| | | ttcactatggatcagtggtgacctaccgctgcaatcctggaagcggagggagaaaggtgtttgagcttgtgggtgagccct |
| | | ccatatactgcaccagcaatgacgatcaagtgggcatctggagcggccccgcccctcagtgcattatacctggtggaggtg |
| | | gatcgggaggtggtggatctggtggaggtggatcgggaggtggtggatctggtggaggtggatcgggaggtggtggatctg |
| | | gtggaggtggatcgggaggtggtggatctggtggaggtggatcgggaggtggtggatctggtggaggtggatcgggaggtg |
| | | gtggatctatcttttgtccaagtcctccagttattcctaatgggagacacacaggaaaacctctggaagtctttccctttggga |
| | | aaacagtaaattacacatgcgacccccacccagacagagggacgagcttcgacctcattggagagagcaccatccgctg |
| | | cacaagtgaccctcaagggaatggggtttggagcagccctgcccctcgctgtggaattctgggtcactgtcaagccccag |
| | | atcattttctgtttgccaagttgaaaacccaaaccaatgcatctgactttcccattgggacatctttaaagtacgaatgccgt |
| | | cctgagtactacgggaggccattctctatcacatgtctagataacctggtctggtcaagtcccaaagatgtctgtaaacgta |
| | | aatcatgtaaaactcctccagatccagtgaatggcatggtgcatgtgatcacagacatccaggttggatccagaatcaact |
| | | attcttgtactacagggcaccgactcattggtcactcatctgctgaatgtatcctctcgggcaatgctgcccattggagcacg |
| | | aagccgccaatttgtcaacgaattccttgtgggctaccccccaccatcgccaatggagatttcattagcaccaacagagag |
| | | aattttcactatggatcagtggtgacctaccgctgcaatcctggaagcggagggagaaaggtgtttgagcttgtgggtgag |
| | | ccctccatatactgcaccagcaatgacgatcaagtgggcatctggagcggcccggcccctcagtgcattatacctggtgga |
| | | ggtggatcgggaggtggtggatctggtggaggtggatcgggaggtggtggatctggtggaggtggatcgggaggtggtgga |
| | | tctggtggaggtggatcgggaggtggtggatctggtggaggtggatcgggaggtggtggatctggtggaggtggatcggga |
| | | ggtggtggatctatcttttgtccaagtcctccagttattcctaatgggagacacacaggaaaacctctggaagtctttcccttt |
| | | ggaaaagcagtaaattacacatgcgacccccacccagacagagggacgagcttcgacctcattggagagagcaccatc |
| | | cgctgcacaagtgaccctcaagggaatggggtttggagcagccctgcccctcgctgtggaattctgggtcactgtcaagcc |
| | | ccagatcattttctgtttgccaagttgaaaacccaaaccaatgcatctgactttcccattgggacatctttaaagtacgaatg |
| | | ccgtcctgagtactacgggaggccattctctatcacatgtctagataacctggtctggtcaagtcccaaagatgtctgtaaa |
| | | cgtaaatcatgtaaaactcctccagatccagtgaatggcatggtgcatgtgatcacagacatccaggttggatccagaatc |
| | | aactattcttgtactacagggcaccgactcattggtcactcatctgctgaatgtatcctctcaggcaatactgcccattggag |
| | | cacgaagccgccaatttgtcaacgaattccttgtgggctacccccaaccatcgccaatggagatttcattagcaccaacag |
| | | agagaattttcactatggatcagtggtgacctaccgctgcaatcttggaagcagagggagaaaggtgtttgagcttgtggg |
| | | tgagccctccatatactgcaccagcaatgacgatcaagtgggcatctggagcggccccgcccctcagtgcattatatagca |
| | | attgaataaaagatctttattttcattagatctgtgtgttggttttttgtgtg |
| 41 | CR2CR1 | ctagttattaatagtaatcaattacggggtcattagttcatagcccatatatggagttccgcgttacataacttacggtaaat |
| | #11B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggccggattagttattattctacccccattgctgttggtaccgtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttggtggaggtggatcgggaggtcgtggatctcaatgcaatgccccagaa |
| | | tggcttccatttgccaggcctaccaacctaactgatgaatttgagtttcccattgggacatatctgaactatgaatgccgccc |
| | | tggttattccggaagaccgttttctatcatctgcctaaaaaactcagtctggactggtgctaaggacaggtgcagacgtaaa |
| | | tcatgtcgtaatcctccagatcctgtgaatggcatggtgcatgtgatcaaaggcatccagttcggatcccaaattaaatatt |
| | | cttgtactaaaggataccgactcattggttcctcgtctgccacatgcatcatctcaggtgatactgtcatttgggataatgaa |
| | | acacctatttgtgacagaattccttgtgggctaccccccaccatcaccaatggagatttcattagcaccaacagagagaatt |
| | | ttcactatggatcagtggtgacctaccgctgcaatcctggaagcggagggagaaaggtgtttgagcttgtgggtgagccct |
| | | ccatatactgcaccagcaatgacgatcaagtgggcatctggagcggccccgcccctcagtgcattatacctggtggaggtg |
| | | gatcgggaggtggtggatctggtggaggtggatcgggaggtggtggatctggtggaggtggatcgatcttttgtccaagtcct |
| | | ccagttattcctaatgggagacacacaggaaaacctctggaagtctttccctttgggaaaacagtaaattacacatgcgac |
| | | ccccacccagacagagggacgagcttcgacctcattggagagagcaccatccgctgcacaagtgaccctcaagggaatg |
| | | gggtttggagcagccctgcccctcgctgtggaattctgggtcactgtcaagccccagatcattttctgtttgccaagttgaaa |
| | | acccaaaccaatgcatctgactttcccattgggacatctttaaagtacgaatgccgtcctgagtactacgggaggccattct |
| | | ctatcacatgtctagataacctggtctggtcaagtcccaaagatgtctgtaaacgtaaatcatgtaaaactcctccagatcc |
| | | agtgaatggcatggtgcatgtgatcacagacatccaggttggatccagaatcaactattcttgtactacagggcaccgact |
| | | cattggtcactcatctgctgaatgtatcctctcgggcaatgctgcccattggagcacgaagccgccaatttgtcaacgaatt |
| | | ccttgtgggctaccccccaccatcgccaatggagatttcattagcaccaacagagagaattttcactatggatcagtggtg |
| | | acctaccgctgcaatcctggaagcggagggagaaaggtgtttgagcttgtgggtgagccctccatatactgcaccagcaa |
| | | tgacgatcaagtgggcatctggagcggcccggcccctcagtgcattatacctggtggaggtggatcgggaggtggtggatc |
| | | tggtggaggtggatcgggaggtggtggatctggtggaggtggatcgatcttttgtccaagtcctccagttattcctaatggga |
| | | gacacacaggaaaacctctggaagtctttccctttggaaaagcagtaaattacacatgcgacccccacccagacagagg |
| | | gacgagcttcgacctcattggagagagcaccatccgctgcacaagtgaccctcaagggaatggggtttggagcagccct |
| | | gcccctcgctgtggaattctgggtcactgtcaagccccagatcattttctgtttgccaagttgaaaacccaaaccaatgcat |
| | | ctgactttcccattgggacatctttaaagtacgaatgccgtcctgagtactacgggaggccattctctatcacatgtctagat |
| | | aacctggtctggtcaagtcccaaagatgtctgtaaacgtaaatcatgtaaaactcctccagatccagtgaatggcatggtg |
| | | catgtgatcacagacatccaggttggatccagaatcaactattcttgtactacagggcaccgactcattggtcactcatctg |
| | | ctgaatgtatcctctcaggcaatactgcccattggagcacgaagccgccaatttgtcaacgaattccttgtgggctaccccc |
| | | aaccatcgccaatggagatttcattagcaccaacagagagaattttcactatggatcagtggtgacctaccgctgcaatctt |
| | | ggaagcagagggagaaaggtgtttgagcttgtgggtgagccctccatatactgcaccagcaatgacgatcaagtgggca |
| | | tctggagcggccccgcccctcagtgcattatatagcaattgaataaaagatctttattttcattagatctgtgtgttggttttttgtgt |
| | | g |
| 42 | CR2CR1 | ctagttattaatagtaatcaattacggggtcattagttcatagcccatatatggagttccgcgttacataacttacggtaaat |
| | #11C | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctagtggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttggtggaggtggatcaggaggtggtggatctcaatgcaatgccccagaat |
| | | ggcttccatttgccaggcctaccaacctaactgatgaatttgagtttcccattgggacatatctgaactatgaatgccgccct |
| | | ggttatagcggaagaccattttctatcatctgcctaaaaaactcagtctggactggtgctaaggacaggtgcagaaggaa |
| | | atcatgtaggaatcctccagatcctgtgaatggcatggtgcatgtgatcaaaggcatccagtttggatcccaaattaaatat |
| | | tcttgtactaaaggataccgcctcattggttccagttctgccacatgcatcatctcaggtgatactgtcatttgggataatga |
| | | aacacctatttgtgacagaattccttgtgggctaccccccaccatcaccaatggagatttcattagcaccaacagagagaa |
| | | ttttcactatggatcagtggtgacctaccgctgcaatcctggaagcggagggagaaaggtgtttgagcttgtgggtgagcc |
| | | ctccatatactgcaccagcaatgatgatcaagtgggcatctggagcggccccgcccctcagtgcattatacctggtggagg |
| | | tggatcaggaggtggtggatctggtggaggtggaagtggaggtggtggatctggtggaggtggatcaggaggtggtggatct |
| | | ggtggaggtggaagtggaggtggtggatctggtggaggtggatcaggaggtggtggatctggtggaggtggaagtggaggt |
| | | ggtggatctatcttttgtccaagtcctccagttattcctaatgggagacacacaggaaaacctctggaagtctttccctttggg |
| | | aaaacagtaaattacacatgtgacccccacccagacagagggactagctttgacctcattggagagagcaccatccgctg |
| | | cacaagtgaccctcaagggaatggggtttggagcagccctgcccctaggtgtggaattctgggtcactgtcaagccccag |
| | | atcattttctgtttgccaagttgaaaacccaaaccaatgcatctgactttcccattgggacatctttaaagtatgaatgccgc |
| | | cctgagtactatgggaggccattctctatcacatgtctagataacctggtctggtcaagtcccaaagatgtctgtaaaagga |
| | | aatcatgtaaaactcctccagatccagtgaatggcatggtgcatgtgatcacagacatccaggttggatccagaatcaact |
| | | attcttgtactactgggcaccgcctcattggtcactcatctgctgaatgtatcctctcaggcaatgctgcccattggagcact |
| | | aagccgccaatttgtcaaaggattccttgtgggctaccccccaccattgccaatggagatttcattagcaccaacagagag |
| | | aattttcactatggatcagtggtgacctaccgctgcaatcctggaagcggagggagaaaggtgtttgagcttgtgggtgag |
| | | ccctccatatactgcaccagcaatgatgatcaagtgggcatctggagcggcccagcccctcagtgcattatacctggtgga |
| | | ggtggaagtggaggtggtggatctggtggaggtggatcaggaggtggtggatctggtggaggtggaagtggaggtggtgga |
| | | tctggtggaggtggatcaggaggtggtggatctggtggaggtggaagtggaggtggtggatctggtggaggtggatcagga |
| | | ggtggtggatctatcttttgtccaagtcctccagttattcctaatgggagacacacaggaaaacctctggaagtctttcccttt |
| | | ggaaaagcagtaaattacacatgtgacccccacccagacagagggactagctttgacctcattggagagagcaccatcc |
| | | gctgcacaagtgaccctcaagggaatggggtttggagcagccctgcccctagatgtggaattctgggtcactgtcaagcc |
| | | ccagatcattttctgtttgccaagttgaaaacccaaaccaatgcatctgactttcccattgggacatctttaaagtatgaatg |
| | | ccgccctgagtactatgggaggccattctctatcacatgtctagataacctggtctggtcaagtcccaaagatgtctgtaaa |
| | | aggaaatcatgtaaaactcctccagatccagtgaatggcatggtgcatgtgatcacagacatccaggttggatccagaat |
| | | caactattcttgtactactgggcaccgcctcattggtcactcatctgctgaatgtatcctctcaggcaatactgcccattgga |
| | | gcactaagccgccaatttgtcaaagaattccttgtgggctacccccaaccattgccaatggagatttcattagcaccaaca |
| | | gagagaattttcactatggatcagtggtgacctaccgctgcaatcttggaagcagagggagaaaggtgtttgagcttgtgg |
| | | gtgagccctccatatactgcaccagcaatgatgatcaagtgggcatctggagcggccccgcccctcagtgcattatatagc |
| | | aattgaataaaagatctttattttcattagatctgtgtgttggttttttgtgtg |
| 43 | CR2CR1 | ctagttattaatagtaatcaattacggggtcattagttcatagcccatatatggagttccgcgttacataacttacggtaaat |
| | #11D | ggcccgcctggctgaccgcccaacgacccccgcccattgacgtcaataatgacgtatgttcccatagtaacgccaatagg |
| | | gactttccattgacgtcaatgggtcgactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctagtggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttggtggaggtggatcaggaggtggtggatctcaatgcaatgccccagaat |
| | | ggcttccatttgccaggcctaccaacctaactgatgaatttgagtttcccattgggacatatctgaactatgaatgccgccct |
| | | ggttatagcggaagaccattttctatcatctgcctaaaaaactcagtctggactggtgctaaggacaggtgcagaaggaa |
| | | atcatgtaggaatcctccagatcctgtgaatggcatggtgcatgtgatcaaaggcatccagtttggatcccaaattaaatat |
| | | tcttgtactaaaggataccgcctcattggttccagttctgccacatgcatcatctcaggtgatactgtcatttgggataatga |
| | | aacacctatttgtgacagaattccttgtgggctaccccccaccatcaccaatggagatttcattagcaccaacagagagaa |
| | | ttttcactatggatcagtggtgacctaccgctgcaatcctggaagcggagggagaaaggtgtttgagcttgtgggtgagcc |
| | | ctccatatactgcaccagcaatgatgatcaagtgggcatctggagcggccccgcccctcagtgcattatacctggtggagg |
| | | tggatcaggaggtggtggatctggtggaggtggaagtggaggtggtggatctggtggaggtggatcaatcttttgtccaagtc |
| | | ctccagttattcctaatgggagacacacaggaaaacctctggaagtctttccctttgggaaaacagtaaattacacatgtg |
| | | acccccacccagacagagggactagctttgacctcattggagagagcaccatccgctgcacaagtgaccctcaagggaa |
| | | tggggtttggagcagccctgcccctaggtgtggaattctgggtcactgtcaagccccagatcattttctgtttgccaagttga |
| | | aaacccaaaccaatgcatctgactttcccattgggacatctttaaagtatgaatgccgccctgagtactatgggaggccatt |
| | | ctctatcacatgtctagataacctggtctggtcaagtcccaaagatgtctgtaaaaggaaatcatgtaaaactcctccagat |
| | | ccagtgaatggcatggtgcatgtgatcacagacatccaggttggatccagaatcaactattcttgtactactgggcaccgc |
| | | ctcattggtcactcatctgctgaatgtatcctctcaggcaatgctgcccattggagcactaagccgccaatttgtcaaaggat |
| | | tccttgtgggctaccccccaccattgccaatggagatttcattagcaccaacagagagaattttcactatggatcagtggtg |
| | | acctaccgctgcaatcctggaagcggagggagaaaggtgtttgagcttgtgggtgagccctccatatactgcaccagcaa |
| | | tgatgatcaagtgggcatctggagcggcccagcccctcagtgcattatacctggtggaggtggaagtggaggtggtggatc |
| | | tggtggaggtggatcaggaggtggtggatctggtggaggtggaagtatcttttgtccaagtcctccagttattcctaatggga |
| | | gacacacaggaaaacctctggaagtctttccctttggaaaagcagtaaattacacatgtgacccccacccagacagagg |
| | | gactagctttgacctcattggagagagcaccatccgctgcacaagtgaccctcaagggaatggggtttggagcagccctg |
| | | cccctagatgtggaattctgggtcactgtcaagccccagatcattttctgtttgccaagttgaaaacccaaaccaatgcatct |
| | | gactttcccattgggacatctttaaagtatgaatgccgccctgagtactatgggaggccattctctatcacatgtctagataa |
| | | cctggtctggtcaagtcccaaagatgtctgtaaaaggaaatcatgtaaaactcctccagatccagtgaatggcatggtgca |
| | | tgtgatcacagacatccaggttggatccagaatcaactattcttgtactactgggcaccgcctcattggtcactcatctgctg |
| | | aatgtatcctctcaggcaatactgcccattggagcactaagccgccaatttgtcaaagaattccttgtgggctacccccaac |
| | | cattgccaatggagatttcattagcaccaacagagagaattttcactatggatcagtggtgacctaccgctgcaatcttgga |
| | | agcagagggagaaaggtgtttgagcttgtgggtgagccctccatatactgcaccagcaatgatgatcaagtgggcatctg |
| | | gagcggccccgcccctcagtgcattatatagcaattgaataaaagatctttattttcattagatctgtgtgttggttttttgtgtg |
| 44 | CR2CR1 | ctagttattaatagtaatcaattacggggtcattagttcatagcccatatatggagttccgcgttacataacttacggtaaat |
| | #12A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccg |
| | | cctatcctaaatggccggattagttattattctacccccattgctgttggtaccgtgataaggtacagttgttcaggtaccttc |
| | | cgcctcattggagaaaaaagtctattatgcataactaaagacaaagtggatggaacctgggataaacctgctcctaaatg |
| | | tgaatatttcaataaatattcttcttgccctgagcccatagtaccaggaggatacaaaattagaggctctacaccctacaga |
| | | catggtgattctgtgacatttgcctgtaaaaccaacttctccatgaacggaaacaagtctgtttggtgtcaagcaaataata |
| | | tgtgggggccgacacgactaccaacctgtgtaagtgttttccctggtggaggtggatcgggaggtggtggatctggtggag |
| | | gtggatcgggaggtggtggatctggtggaggtggatcgggaggtggtggatctcaatgcaatgccccagaatggcttcca |
| | | tttgccaggcctaccaacctaactgatgaatttgagtttcccattgggacatatctgaactatgaatgccgccctggttattc |
| | | cggaagaccgttttctatcatctgcctaaaaaactcagtctggactggtgctaaggacaggtgcagacgtaaatcatgtcg |
| | | taatcctccagatcctgtgaatggcatggtgcatgtgatcaaaggcatccagttcggatcccaaattaaatattcttgtacta |
| | | aaggataccgactcattggttcctcgtctgccacatgcatcatctcaggtgatactgtcatttgggataatgaaacacctatt |
| | | tgtgacagaattccttgtgggctaccccccaccatcaccaatggagatttcattagcaccaacagagagaattttcactatg |
| | | gatcagtggtgacctaccgctgcaatcctggaagcggagggagaaaggtgtttgagcttgtgggtgagccctccatatact |
| | | gcaccagcaatgacgatcaagtgggcatctggagcggccccgcccctcagtgcattatacctaacaaatgcacgcctcca |
| | | aatgtggaaaatggaatattggtatctgacaacagaagcttattttccttaaatgaagttgtggagtttaggtgtcagcctg |
| | | gctttgtcatgaaaggaccccgccgtgtgaagtgccaggccctgaacaaatgggagccggagctaccaagctgctccag |
| | | ggtatgtcagccacctccagatgtcctgcatgctgagcgtacccaaagggacaaggacaacttttcacctgggcaggaag |
| | | tgttctacagctgtgagcccggctacgacctcagaggggctgcgtctatgcgctgcacaccccagggagactggagccct |
| | | gcagcccccacatgtgaagtgatcttttgtccaagtcctccagttattcctaatgggagacacacaggaaaacctctggaa |
| | | gtctttccctttgggaaaacagtaaattacacatgcgacccccacccagacagagggacgagcttcgacctcattggaga |
| | | gagcaccatccgctgcacaagtgaccctcaagggaatggggtttggagcagccctgcccctcgctgtggaattctgggtc |
| | | actgtcaagccccagatcattttctgtttgccaagttgaaaacccaaaccaatgcatctgactttcccattgggacatcttta |
| | | aagtacgaatgccgtcctgagtactacgggaggccattctctatcacatgtctagataacctggtctggtcaagtcccaaa |
| | | gatgtctgtaaacgtaaatcatgtaaaactcctccagatccagtgaatggcatggtgcatgtgatcacagacatccaggtt |
| | | ggatccagaatcaactattcttgtactacagggcaccgactcattggtcactcatctgctgaatgtatcctctcgggcaatgc |
| | | tgcccattggagcacgaagccgccaatttgtcaacgaattccttgtgggctaccccccaccatcgccaatggagatttcatt |
| | | agcaccaacagagagaattttcactatggatcagtggtgacctaccgctgcaatcctggaagcggagggagaaaggtgt |
| | | ttgagcttgtgggtgagccctccatatactgcaccagcaatgacgatcaagtgggcatctggagcggcccggcccctcagt |
| | | gcattatacctaacaaatgcacgcctccaaatgtggaaaatggaatattggtatctgacaacagaagcttattttccttaaa |
| | | tgaagttgtggagtttaggtgtcagcctggctttgtcatgaaaggaccccgccgtgtgaagtgccaggccctgaacaaatg |
| | | ggagccggagctaccaagctgctccagggtatgtcagccacctccagatgtcctgcatgctgagcgtacccaaagggac |
| | | aaggacaacttttcacccgggcaggaagtgttctacagctgtgagcccggctatgacctcagaggggctgcgtctatgcg |
| | | ctgcacaccccagggagactggagccctgcagcccccacatgtgaagtgatcttttgtccaagtcctccagttattcctaat |
| | | gggagacacacaggaaaacctctggaagtctttccctttggaaaagcagtaaattacacatgcgacccccacccagaca |
| | | gagggacgagcttcgacctcattggagagagcaccatccgctgcacaagtgaccctcaagggaatggggtttggagcag |
| | | ccctgcccctcgctgtggaattctgggtcactgtcaagccccagatcattttctgtttgccaagttgaaaacccaaaccaatg |
| | | catctgactttcccattgggacatctttaaagtacgaatgccgtcctgagtactacgggaggccattctctatcacatgtcta |
| | | gataacctggtctggtcaagtcccaaagatgtctgtaaacgtaaatcatgtaaaactcctccagatccagtgaatggcatg |
| | | gtgcatgtgatcacagacatccaggttggatccagaatcaactattcttgtactacagggcaccgactcattggtcactcat |
| | | ctgctgaatgtatcctctcaggcaatactgcccattggagcacgaagccgccaatttgtcaacgaattccttgtgggctacc |
| | | cccaaccatcgccaatggagatttcattagcaccaacagagagaattttcactatggatcagtggtgacctaccgctgcaa |
| | | tcttggaagcagagggagaaaggtgtttgagcttgtgggtgagccctccatatactgcaccagcaatgacgatcaagtgg |
| | | gcatctggagcggccccgcccctcagtgcattatatagcaattgaataaaagatctttattttcattagatctgtgtgttggtttttt |
| | | gtgtg |
| 45 | CR2CR1 | ctagttattaatagtaatcaattacggggtcattagttcatagcccatatatggagttccgcgttacataacttacggtaaat |
| | #12B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggggg |
| | | agtcgctgcgacgctgccttcgccccgtgccccgctccgccgccgcctcgcgccgcccgccccggctctgactgaccgcgt |
| | | tactcccacaggtgagcggggggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccg |
| | | cctatcctaaatggccggattagttattattctacccccattgctgttggtaccgtgataaggtacagttgttcaggtaccttc |
| | | cgcctcattggagaaaaaagtctattatgcataactaaagacaaagtggatggaacctgggataaacctgctcctaaatg |
| | | tgaatatttcaataaatattcttcttgccctgagcccatagtaccaggaggatacaaaattagaggctctacaccctacaga |
| | | catggtgattctgtgacatttgcctgtaaaaccaacttctccatgaacggaaacaagtctgtttggtgtcaagcaaataata |
| | | tgtgggggccgacacgactaccaacctgtgtaagtgttttccctggtggaggtggatcgggaggtggtggatctggtggag |
| | | gtggatcgcaatgcaatgccccagaatggcttccatttgccaggcctaccaacctaactgatgaatttgagtttcccattgg |
| | | gacatatctgaactatgaatgccgccctggttattccggaagaccgttttctatcatctgcctaaaaaactcagtctggactg |
| | | gtgctaaggacaggtgcagacgtaaatcatgtcgtaatcctccagatcctgtgaatggcatggtgcatgtgatcaaaggc |
| | | atccagttcggatcccaaattaaatattcttgtactaaaggataccgactcattggttcctcgtctgccacatgcatcatctca |
| | | ggtgatactgtcatttgggataatgaaacacctatttgtgacagaattccttgtgggctaccccccaccatcaccaatggag |
| | | atttcattagcaccaacagagagaattttcactatggatcagtggtgacctaccgctgcaatcctggaagcggagggaga |
| | | aaggtgtttgagcttgtgggtgagccctccatatactgcaccagcaatgacgatcaagtgggcatctggagcggccccgc |
| | | ccctcagtgcattatacctaacaaatgcacgcctccaaatgtggaaaatggaatattggtatctgacaacagaagcttattt |
| | | tccttaaatgaagttgtggagtttaggtgtcagcctggctttgtcatgaaaggaccccgccgtgtgaagtgccaggccctga |
| | | acaaatgggagccggagctaccaagctgctccagggtatgtcagccacctccagatgtcctgcatgctgagcgtacccaa |
| | | agggacaaggacaacttttcacctgggcaggaagtgttctacagctgtgagcccggctacgacctcagaggggctgcgtc |
| | | tatgcgctgcacaccccagggagactggagccctgcagcccccacatgtgaagtgatcttttgtccaagtcctccagttatt |
| | | cctaatgggagacacacaggaaaacctctggaagtctttccctttgggaaaacagtaaattacacatgcgacccccaccc |
| | | agacagagggacgagcttcgacctcattggagagagcaccatccgctgcacaagtgaccctcaagggaatggggtttgg |
| | | agcagccctgcccctcgctgtggaattctgggtcactgtcaagccccagatcattttctgtttgccaagttgaaaacccaaa |
| | | ccaatgcatctgactttcccattgggacatctttaaagtacgaatgccgtcctgagtactacgggaggccattctctatcaca |
| | | tgtctagataacctggtctggtcaagtcccaaagatgtctgtaaacgtaaatcatgtaaaactcctccagatccagtgaatg |
| | | gcatggtgcatgtgatcacagacatccaggttggatccagaatcaactattcttgtactacagggcaccgactcattggtca |
| | | ctcatctgctgaatgtatcctctcgggcaatgctgcccattggagcacgaagccgccaatttgtcaacgaattccttgtggg |
| | | ctaccccccaccatcgccaatggagatttcattagcaccaacagagagaattttcactatggatcagtggtgacctaccgc |
| | | tgcaatcctggaagcggagggagaaaggtgtttgagcttgtgggtgagccctccatatactgcaccagcaatgacgatca |
| | | agtgggcatctggagcggcccggcccctcagtgcattatacctaacaaatgcacgcctccaaatgtggaaaatggaatat |
| | | tggtatctgacaacagaagcttattttccttaaatgaagttgtggagtttaggtgtcagcctggctttgtcatgaaaggaccc |
| | | cgccgtgtgaagtgccaggccctgaacaaatgggagccggagctaccaagctgctccagggtatgtcagccacctccag |
| | | atgtcctgcatgctgagcgtacccaaagggacaaggacaacttttcacccgggcaggaagtgttctacagctgtgagccc |
| | | ggctatgacctcagaggggctgcgtctatgcgctgcacaccccagggagactggagccctgcagcccccacatgtgaagt |
| | | gatcttttgtccaagtcctccagttattcctaatgggagacacacaggaaaacctctggaagtctttccctttggaaaagca |
| | | gtaaattacacatgcgacccccacccagacagagggacgagcttcgacctcattggagagagcaccatccgctgcacaa |
| | | gtgaccctcaagggaatggggtttggagcagccctgcccctcgctgtggaattctgggtcactgtcaagccccagatcattt |
| | | tctgtttgccaagttgaaaacccaaaccaatgcatctgactttcccattgggacatctttaaagtacgaatgccgtcctgagt |
| | | actacgggaggccattctctatcacatgtctagataacctggtctggtcaagtcccaaagatgtctgtaaacgtaaatcatg |
| | | taaaactcctccagatccagtgaatggcatggtgcatgtgatcacagacatccaggttggatccagaatcaactattcttgt |
| | | actacagggcaccgactcattggtcactcatctgctgaatgtatcctctcaggcaatactgcccattggagcacgaagccg |
| | | ccaatttgtcaacgaattccttgtgggctacccccaaccatcgccaatggagatttcattagcaccaacagagagaattttc |
| | | actatggatcagtggtgacctaccgctgcaatcttggaagcagagggagaaaggtgtttgagcttgtgggtgagccctcc |
| | | atatactgcaccagcaatgacgatcaagtgggcatctggagcggccccgcccctcagtgcattatatagcaattgaataaa |
| | | agatctttattttcattagatctgtgtgttggttttttgtgtg |
| 46 | CR2CR1 | ctagttattaatagtaatcaattacggggtcattagttcatagcccatatatggagttccgcgttacataacttacggtaaat |
| | #12C | ggcccgcctggctgaccgcccaacgacccccgcccattgacgtcaataatgacgtatgttcccatagtaacgccaatagg |
| | | gactttccattgacgtcaatgggggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggcggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccg |
| | | cctatcctaaatggcaggattagttattattctacccccattgctgttggtacagtgataaggtacagttgttcaggtaccttc |
| | | cgcctcattggagaaaaaagtctattatgcataactaaagacaaagtggatggaacctgggataaacctgctcctaaatg |
| | | tgaatatttcaataaatattcttcttgccctgagcccatagtaccaggaggatacaaaattagaggctctacaccctacaga |
| | | catggtgattctgtgacatttgcctgtaaaaccaacttctccatgaatggaaacaagtctgtttggtgtcaagcaaataatat |
| | | gtgggggccaacaaggctaccaacctgtgtaagtgttttccctggtggaggtggaagtggaggtggtggatctggtggag |
| | | gtggaagtggaggtggtggatctggtggaggtggaagtggaggtggtggatctcaatgcaatgccccagaatggcttcca |
| | | tttgccaggcctaccaacctaactgatgaatttgagtttcccattgggacatatctgaactatgaatgccgccctggttatag |
| | | cggaagaccattttctatcatctgcctaaaaaactcagtctggactggtgctaaggacaggtgcagaaggaaatcatgta |
| | | ggaatcctccagatcctgtgaatggcatggtgcatgtgatcaaaggcatccagtttggatcccaaattaaatattcttgtact |
| | | aaaggataccgcctcattggttccagttctgccacatgcatcatctcaggtgatactgtcatttgggataatgaaacacctat |
| | | ttgtgacagaattccttgtgggctaccccccaccatcaccaatggagatttcattagcaccaacagagagaattttcactat |
| | | ggatcagtggtgacctaccgctgcaatcctggaagcggagggagaaaggtgtttgagcttgtgggtgagccctccatata |
| | | ctgcaccagcaatgatgatcaagtgggcatctggagcggccccgcccctcagtgcattatacctaacaaatgcactcctcc |
| | | aaatgtggaaaatggaatattggtatctgacaacagaagcttattttccttaaatgaagttgtggagtttaggtgtcagcct |
| | | ggctttgtcatgaaaggaccccgcagggtgaagtgccaggccctgaacaaatgggagccagagctaccaagctgctcca |
| | | gggtatgtcagccacctccagatgtcctgcatgctgagcgcacccaaagggacaaggacaacttttcacctgggcaggaa |
| | | gtgttctacagctgtgagcctggctatgacctcagaggggctgcatctatgcgctgcacaccccagggagactggagccct |
| | | gcagcccccacatgtgaagtgatcttttgtccaagtcctccagttattcctaatgggagacacacaggaaaacctctggaa |
| | | gtctttccctttgggaaaacagtaaattacacatgtgacccccacccagacagagggactagctttgacctcattggagag |
| | | agcaccatccgctgcacaagtgaccctcaagggaatggggtttggagcagccctgcccctaggtgtggaattctgggtca |
| | | ctgtcaagccccagatcattttctgtttgccaagttgaaaacccaaaccaatgcatctgactttcccattgggacatctttaa |
| | | agtatgaatgccgccctgagtactatgggaggccattctctatcacatgtctagataacctggtctggtcaagtcccaaaga |
| | | tgtctgtaaaaggaaatcatgtaaaactcctccagatccagtgaatggcatggtgcatgtgatcacagacatccaggttgg |
| | | atccagaatcaactattcttgtactactgggcaccgcctcattggtcactcatctgctgaatgtatcctcagtggcaatgctg |
| | | cccattggagcactaagccgccaatttgtcaaaggattccttgtgggctaccccccaccattgccaatggagatttcattag |
| | | caccaacagagagaattttcactatggatcagtggtgacctaccgctgcaatcctggaagcggagggagaaaggtgtttg |
| | | agcttgtgggtgagccctccatatactgcaccagcaatgatgatcaagtgggcatctggagcggcccagcccctcagtgc |
| | | attatacctaacaaatgcactcctccaaatgtggaaaatggaatattggtatctgacaacagaagcttattttccttaaatg |
| | | aagttgtggagtttaggtgtcagcctggctttgtcatgaaaggaccccgcagggtgaagtgccaggccctgaacaaatgg |
| | | gagccagagctaccaagctgctccagggtatgtcagccacctccagatgtcctgcatgctgagcgcacccaaagggaca |
| | | aggacaacttttcacctgggcaggaagtgttctacagctgtgagcctggctatgacctcagaggggctgcatctatgcgct |
| | | gcacaccccagggagactggagccctgcagcccccacatgtgaagtgatcttttgtccaagtcctccagttattcctaatgg |
| | | gagacacacaggaaaacctctggaagtctttccctttggaaaagcagtaaattacacatgtgacccccacccagacaga |
| | | gggactagctttgacctcattggagagagcaccatccgctgcacaagtgaccctcaagggaatggggtttggagcagccc |
| | | tgcccctaggtgtggaattctgggtcactgtcaagccccagatcattttctgtttgccaagttgaaaacccaaaccaatgca |
| | | tctgactttcccattgggacatctttaaagtatgaatgccgccctgagtactatgggaggccattctctatcacatgtctagat |
| | | aacctggtctggtcaagtcccaaagatgtctgtaaaagaaaatcatgtaaaactcctccagatccagtgaatggcatggtg |
| | | catgtgatcacagacatccaggttggatccagaatcaactattcttgtactactgggcaccgcctcattggtcactcatctgc |
| | | tgaatgtatcctctcaggcaatactgcccattggagcactaagccgccaatttgtcaaaggattccttgtgggctaccccca |
| | | accattgccaatggagatttcattagcaccaacagagagaattttcactatggatcagtggtgacctaccgctgcaatcttg |
| | | gaagcagagggagaaaggtgtttgagcttgtgggtgagccctccatatactgcaccagcaatgatgatcaagtgggcatc |
| | | tggagcggccccgcccctcagtgcattatatagcaattgaataaaagatctttattttcattagatctgtgtgttggttttttgtgtg |
| 47 | CR2CR1 | ctagttattaatagtaatcaattacggggtcattagttcatagcccatatatggagttccgcgttacataacttacggtaaat |
| | #12D | ggcccgcctggctgaccgcccaacgacccccgcccattgacgtcaataatgacgtatgttcccatagtaacgccaatagg |
| | | gactttccattgacgtcaatgggggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcggcgggggg |
| | | agtcgctgcgacgctgccttcgccccgtgccccgctccgccgccgcctcgcgccgcccgccccggctctgactgaccgcgt |
| | | tactcccacaggtgagcggggggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccg |
| | | cctatcctaaatggcaggattagttattattctacccccattgctgttggtacagtgataaggtacagttgttcaggtaccttc |
| | | cgcctcattggagaaaaaagtctattatgcataactaaagacaaagtggatggaacctgggataaacctgctcctaaatg |
| | | tgaatatttcaataaatattcttcttgccctgagcccatagtaccaggaggatacaaaattagaggctctacaccctacaga |
| | | catggtgattctgtgacatttgcctgtaaaaccaacttctccatgaatggaaacaagtctgtttggtgtcaagcaaataatat |
| | | gtgggggccaacaaggctaccaacctgtgtaagtgttttccctggtggaggtggaagtggaggtggtggatctggtggag |
| | | gtggaagtcaatgcaatgccccagaatggcttccatttgccaggcctaccaacctaactgatgaatttgagtttcccattgg |
| | | gacatatctgaactatgaatgccgccctggttatagcggaagaccattttctatcatctgcctaaaaaactcagtctggact |
| | | ggtgctaaggacaggtgcagaaggaaatcatgtaggaatcctccagatcctgtgaatggcatggtgcatgtgatcaaag |
| | | gcatccagtttggatcccaaattaaatattcttgtactaaaggataccgcctcattggttccagttctgccacatgcatcatct |
| | | caggtgatactgtcatttgggataatgaaacacctatttgtgacagaattccttgtgggctaccccccaccatcaccaatgg |
| | | agatttcattagcaccaacagagagaattttcactatggatcagtggtgacctaccgctgcaatcctggaagcggaggga |
| | | gaaaggtgtttgagcttgtgggtgagccctccatatactgcaccagcaatgatgatcaagtgggcatctggagcggcccc |
| | | gcccctcagtgcattatacctaacaaatgcactcctccaaatgtggaaaatggaatattggtatctgacaacagaagctta |
| | | ttttccttaaatgaagttgtggagtttaggtgtcagcctggctttgtcatgaaaggaccccgcagggtgaagtgccaggccc |
| | | tgaacaaatgggagccagagctaccaagctgctccagggtatgtcagccacctccagatgtcctgcatgctgagcgcacc |
| | | caaagggacaaggacaacttttcacctgggcaggaagtgttctacagctgtgagcctggctatgacctcagaggggctgc |
| | | atctatgcgctgcacaccccagggagactggagccctgcagcccccacatgtgaagtgatcttttgtccaagtcctccagtt |
| | | attcctaatgggagacacacaggaaaacctctggaagtctttccctttgggaaaacagtaaattacacatgtgacccccac |
| | | ccagacagagggactagctttgacctcattggagagagcaccatccgctgcacaagtgaccctcaagggaatggggtttg |
| | | gagcagccctgcccctaggtgtggaattctgggtcactgtcaagccccagatcattttctgtttgccaagttgaaaacccaa |
| | | accaatgcatctgactttcccattgggacatctttaaagtatgaatgccgccctgagtactatgggaggccattctctatcac |
| | | atgtctagataacctggtctggtcaagtcccaaagatgtctgtaaaaggaaatcatgtaaaactcctccagatccagtgaa |
| | | tggcatggtgcatgtgatcacagacatccaggttggatccagaatcaactattcttgtactactgggcaccgcctcattggt |
| | | cactcatctgctgaatgtatcctcagtggcaatgctgcccattggagcactaagccgccaatttgtcaaaggattccttgtg |
| | | ggctaccccccaccattgccaatggagatttcattagcaccaacagagagaattttcactatggatcagtggtgacctacc |
| | | gctgcaatcctggaagcggagggagaaaggtgtttgagcttgtgggtgagccctccatatactgcaccagcaatgatgat |
| | | caagtgggcatctggagcggcccagcccctcagtgcattatacctaacaaatgcactcctccaaatgtggaaaatggaat |
| | | attggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccagggtatgtcagccacctcc |
| | | agatgtcctgcatgctgagcgcacccaaagggacaaggacaacttttcacctgggcaggaagtgttctacagctgtgagc |
| | | ctggctatgacctcagaggggctgcatctatgcgctgcacaccccagggagactggagccctgcagcccccacatgtgaa |
| | | gtgatcttttgtccaagtcctccagttattcctaatgggagacacacaggaaaacctctggaagtctttccctttggaaaagc |
| | | agtaaattacacatgtgacccccacccagacagagggactagctttgacctcattggagagagcaccatccgctgcacaa |
| | | gtgaccctcaagggaatggggtttggagcagccctgcccctaggtgtggaattctgggtcactgtcaagccccagatcatt |
| | | ttctgtttgccaagttgaaaacccaaaccaatgcatctgactttcccattgggacatctttaaagtatgaatgccgccctgag |
| | | tactatgggaggccattctctatcacatgtctagataacctggtctggtcaagtcccaaagatgtctgtaaaagaaaatcat |
| | | gtaaaactcctccagatccagtgaatggcatggtgcatgtgatcacagacatccaggttggatccagaatcaactattctt |
| | | gtactactgggcaccgcctcattggtcactcatctgctgaatgtatcctctcaggcaatactgcccattggagcactaagcc |
| | | gccaatttgtcaaaggattccttgtgggctacccccaaccattgccaatggagatttcattagcaccaacagagagaatttt |
| | | cactatggatcagtggtgacctaccgctgcaatcttggaagcagagggagaaaggtgtttgagcttgtgggtgagccctcc |
| | | atatactgcaccagcaatgatgatcaagtgggcatctggagcggccccgcccctcagtgcattatatagcaattgaataaa |
| | | agatctttattttcattagatctgtgtgttggttttttgtgtg |
| 48 | CR2CR1 | ctagttattaatagtaatcaattacggggtcattagttcatagcccatatatggagttccgcgttacataacttacggtaaat |
| | #1A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggccggattagttattattctacccccattgctgttggtaccgtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttcaatgcaatgccccagaatggcttccatttgccaggcctaccaaccta |
| | | actgatgaatttgagtttcccattgggacatatctgaactatgaatgccgccctggttattccggaagaccgttttctatcatc |
| | | tgcctaaaaaactcagtctggactggtgctaaggacaggtgcagacgtaaatcatgtcgtaatcctccagatcctgtgaat |
| | | ggcatggtgcatgtgatcaaaggcatccagttcggatcccaaattaaatattcttgtactaaaggataccgactcattggtt |
| | | cctcgtctgccacatgcatcatctcaggtgatactgtcatttgggataatgaaacacctatttgtgacagaattccttgtggg |
| | | ctaccccccaccatcaccaatggagatttcattagcaccaacagagagaattttcactatggatcagtggtgacctaccgc |
| | | tgcaatcctggaagcggagggagaaaggtgtttgagcttgtgggtgagccctccatatactgcaccagcaatgacgatca |
| | | agtgggcatctggagcggccccgcccctcagtgcattatacctaacaaatgcacgcctccaaatgtggaaaatggaatat |
| | | tggtatctgacaacagaagcttattttccttaaatgaagttgtggagtttaggtgtcagcctggctttgtcatgaaaggaccc |
| | | cgccgtgtgaagtgccaggccctgaacaaatgggagccggagctaccaagctgctccagggtatgtcagccacctccag |
| | | atgtcctgcatgctgagcgtacccaaagggacaaggacaacttttcacctgggcaggaagtgttctacagctgtgagccc |
| | | ggctacgacctcagaggggctgcgtctatgcgctgcacaccccagggagactggagccctgcagcccccacatgtgaag |
| | | tgaaatcctgtgatgacttcatgggccaacttcttaatggccgtgtgctatttccagtaaatctccagcttggagcaaaagtg |
| | | gattttgtttgtgatgaaggatttcaattaaaaggcagctctgctagttactgtgtcttggctggaatggaaagcctttggaa |
| | | tagcagtgttccagtgtgtgaacaaatcttttgtccaagtcctccagttattcctaatgggagacacacaggaaaacctctg |
| | | gaagtctttccctttgggaaaacagtaaattacacatgcgacccccacccagacagagggacgagcttcgacctcattgg |
| | | agagagcaccatccgctgcacaagtgaccctcaagggaatggggtttggagcagccctgcccctcgctgtggaattctgg |
| | | gtcactgtcaagccccagatcattttctgtttgccaagttgaaaacccaaaccaatgcatctgactttcccattgggacatct |
| | | ttaaagtacgaatgccgtcctgagtactacgggaggccattctctatcacatgtctagataacctggtctggtcaagtccca |
| | | aagatgtctgtaaacgtaaatcatgtaaaactcctccagatccagtgaatggcatggtgcatgtgatcacagacatccagg |
| | | ttggatccagaatcaactattcttgtactacagggcaccgactcattggtcactcatctgctgaatgtatcctctcgggcaat |
| | | gctgcccattggagcacgaagccgccaatttgtcaacgaattccttgtgggctaccccccaccatcgccaatggagatttc |
| | | attagcaccaacagagagaattttcactatggatcagtggtgacctaccgctgcaatcctggaagcggagggagaaagg |
| | | tgtttgagcttgtgggtgagccctccatatactgcaccagcaatgacgatcaagtgggcatctggagcggcccggcccctc |
| | | agtgcattatacctaacaaatagcaattgctgtgccttctagttgccagccatctgttgtttgcccctcccccgtgccttccttgacc |
| | | ctggaaggtgccactcccactgtcctttcctaataaaatgaggaaattgcatcgcattgtctgagtaggtgtcattctattctggg |
| | | gggtggggggggcaggacagcaagggggaggattgggaagacaatagcaggcatgctggggatgcggtgggctctatg |
| 49 | CR2CR1 | ctagttattaatagtaatcaattacggggtcattagttcatagcccatatatggagttccgcgttacataacttacggtaaat |
| | #1B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtcatctggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtagatgtgtaattgctggacagggagttg |
| | | cttggaccaaaatgccagtatgtgaagaaatttttcaatgcaatgccccagaatggcttccatttgccaggcctaccaacctaa |
| | | ctgatgaatttgagtttcccattgggacatatctgaactatgaatgccgccctggttatagcggaagaccattttctatcatct |
| | | gcctaaaaaactcagtctggactggtgctaaggacaggtgcagaaggaaatcatgtagaaatcctccagatcctgtgaat |
| | | ggcatggtgcatgtgatcaaaggcatccagtttggatcccaaattaaatattcttgtactaaaggataccgcctcattggttc |
| | | ctcatctgccacatgcatcatctcaggtgatactgtcatttgggataatgaaacacctatttgtgacagaattccttgtgggc |
| | | taccccccaccatcaccaatggagatttcattagcaccaacagagagaattttcactatggatcagtggtgacctaccgct |
| | | gcaatcctggaagcggagggagaaaggtgtttgagcttgtgggtgagccctccatatactgcaccagcaatgatgatcaa |
| | | gtgggcatctggagcggccccgcccctcagtgcattatacctaacaaatgcactcctccaaatgtggaaaatggaatattg |
| | | gtatctgacaacagaagcttattttccttaaatgaagttgtggagtttaggtgtcagcctggctttgtcatgaaaggaccccg |
| | | cagggtgaagtgccaggccctgaacaaatgggagccagagctaccaagctgctccagggtatgtcagccacctccagat |
| | | gtcctgcatgctgagcgcacccaaagggacaaggacaacttttcacctgggcaggaagtgttctacagctgtgagcctgg |
| | | ctatgacctcagaggggctgcatctatgcgctgcacaccccagggagactggagccctgcagcccccacatgtgaagtga |
| | | aatcctgtgatgacttcatgggccaacttcttaatggcagagtgctatttccagtaaatctccagcttggagcaaaagtgga |
| | | ttttgtttgtgatgaaggatttcaattaaaaggcagctctgctagttactgtgtcttggctggaatggaaagcctttggaata |
| | | gcagtgttccagtgtgtgaacaaatcttttgtccaagtcctccagttattcctaatgggagacacacaggaaaacctctgga |
| | | agtctttccctttgggaaaacagtaaattacacatgtgacccccacccagacagagggactagctttgacctcattggaga |
| | | gagcaccatccgctgcacaagtgaccctcaagggaatggggtttggagcagccctgcccctaggtgtggaattctgggtc |
| | | actgtcaagccccagatcattttctgtttgccaagttgaaaacccaaaccaatgcatctgactttcccattgggacatcttta |
| | | aagtatgaatgccgccctgagtactatgggaggccattctctatcacatgtctagataacctggtctggtcaagtcccaaag |
| | | atgtctgtaaaagaaaatcatgtaaaactcctccagatccagtgaatggcatggtgcatgtgatcacagacatccaggttg |
| | | gatccagaatcaactattcttgtacaactgggcaccgcctcattggtcactcatctgctgaatgtatcctctcaggcaatgct |
| | | gcccattggagcactaagccgccaatttgtcaaaggattccttgtgggctaccccccaccattgccaatggagatttcatta |
| | | gcaccaacagagagaattttcactatggatcagtggtgacctaccgctgcaatcctggaagcggagggagaaaggtgttt |
| | | gagcttgtgggtgagccctccatatactgcaccagcaatgatgatcaagtgggcatctggagcggcccagcccctcagtg |
| | | cattatacctaacaaatagcaattgctgtgccttctagttgccagccatctgttgtttgcccctcccccgtgccttccttgaccctgg |
| | | aaggtgccactcccactgtcctttcctaataaaatgaggaaattgcatcgcattgtctgagtaggtgtcattctattctggggggt |
| | | ggggggggcaggacagcaagggggaggattgggaagacaatagcaggcatgctggggatgcggtgggctctatg |
| 50 | CR2CR1 | ctagttattaatagtaatcaattacggggtcattagttcatagcccatatatggagttccgcgttacataacttacggtaaat |
| | #1C | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttcaatgcaatgccccagaatggcttccatttgccaggcctaccaaccta |
| | | actgatgaatttgagtttcccattgggacatatctgaactatgaatgccgccctggttattccggaagaccgttttctatcatc |
| | | tgcctaaaaaactcagtctggactggtgctaaggacaggtgcagacgtaaatcatgtcgtaatcctccagatcctgtgaat |
| | | ggcatggtgcatgtgatcaaaggcatccagttcggatcccaaattaaatattcttgtactaaaggataccgactcattggtt |
| | | cctcgtctgccacatgcatcatctcaggtgatactgtcatttgggataatgaaacacctatttgtgacagaattccttgtggg |
| | | ctaccccccaccatcaccaatggagatttcattagcaccaacagagagaattttcactatggatcagtggtgacctaccgc |
| | | tgcaatcctggaagcggagggagaaaggtgtttgagcttgtgggtgagccctccatatactgcaccagcaatgacgatca |
| | | agtgggcatctggagcggccccgcccctcagtgcattatacctaacaaatgcacgcctccaaatgtggaaaatggaatat |
| | | tggtatctgacaacagaagcttattttccttaaatgaagttgtggagtttaggtgtcagcctggctttgtcatgaaaggaccc |
| | | cgccgtgtgaagtgccaggccctgaacaaatgggagccggagctaccaagctgctccagggtatgtcagccacctccag |
| | | atgtcctgcatgctgagcgtacccaaagggacaaggacaacttttcacctgggcaggaagtgttctacagctgtgagccc |
| | | ggctacgacctcagaggggctgcgtctatgcgctgcacaccccagggagactggagccctgcagcccccacatgtgaag |
| | | tgaaatcctgtgatgacttcatgggccaacttcttaatggccgtgtgctatttccagtaaatctccagcttggagcaaaagtg |
| | | gattttgtttgtgatgaaggatttcaattaaaaggcagctctgctagttactgtgtcttggctggaatggaaagcctttggaa |
| | | tagcagtgttccagtgtgtgaacaaatcttttgtccaagtcctccagttattcctaatgggagacacacaggaaaacctctg |
| | | gaagtctttccctttgggaaaacagtaaattacacatgcgacccccacccagacagagggacgagcttcgacctcattgg |
| | | agagagcaccatccgctgcacaagtgaccctcaagggaatggggtttggagcagccctgcccctcgctgtggaattctgg |
| | | gtcactgtcaagccccagatcattttctgtttgccaagttgaaaacccaaaccaatgcatctgactttcccattgggacatct |
| | | ttaaagtacgaatgccgtcctgagtactacgggaggccattctctatcacatgtctagataacctggtctggtcaagtccca |
| | | aagatgtctgtaaacgtaaatcatgtaaaactcctccagatccagtgaatggcatggtgcatgtgatcacagacatccagg |
| | | ttggatccagaatcaactattcttgtactacagggcaccgactcattggtcactcatctgctgaatgtatcctctcgggcaat |
| | | gctgcccattggagcacgaagccgccaatttgtcaacgaattccttgtgggctaccccccaccatcgccaatggagatttc |
| | | attagcaccaacagagagaattttcactatggatcagtggtgacctaccgctgcaatcctggaagcggagggagaaagg |
| | | tgtttgagcttgtgggtgagccctccatatactgcaccagcaatgacgatcaagtgggcatctggagcggcccggcccctc |
| | | agtgcattatatagcaattgaataaaagatctttattttcattagatctgtgtgttggttttttgtgtg |
| 51 | CR2CR1 | ctagttattaatagtaatcaattacggggtcattagttcatagcccatatatggagttccgcgttacataacttacggtaaat |
| | #2A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttggtggaggtggatcgggaggtggtggatctcaatgcaatgccccagaa |
| | | tggcttccatttgccaggcctaccaacctaactgatgaatttgagtttcccattgggacatatctgaactatgaatgccgccc |
| | | tggttattccggaagaccgttttctatcatctgcctaaaaaactcagtctggactggtgctaaggacaggtgcagacgtaaa |
| | | tcatgtcgtaatcctccagatcctgtgaatggcatggtgcatgtgatcaaaggcatccagttcggatcccaaattaaatatt |
| | | cttgtactaaaggataccgactcattggttcctcgtctgccacatgcatcatctcaggtgatactgtcatttgggataatgaa |
| | | acacctatttgtgacagaattccttgtgggctaccccccaccatcaccaatggagatttcattagcaccaacagagagaatt |
| | | ttcactatggatcagtggtgacctaccgctgcaatcctggaagcggagggagaaaggtgtttgagcttgtgggtgagccct |
| | | ccatatactgcaccagcaatgacgatcaagtgggcatctggagcggccccgcccctcagtgcattatacctaacaaatgc |
| | | acgcctccaaatgtggaaaatggaatattggtatctgacaacagaagcttattttccttaaatgaagttgtggagtttaggt |
| | | gtcagcctggctttgtcatgaaaggaccccgccgtgtgaagtgccaggccctgaacaaatgggagccggagctaccaag |
| | | ctgctccagggtatgtcagccacctccagatgtcctgcatgctgagcgtacccaaagggacaaggacaacttttcacctgg |
| | | gcaggaagtgttctacagctgtgagcccggctacgacctcagaggggctgcgtctatgcgctgcacaccccagggagact |
| | | ggagccctgcagcccccacatgtgaagtgaaatcctgtgatgacttcatgggccaacttcttaatggccgtgtgctatttcc |
| | | agtaaatctccagcttggagcaaaagtggattttgtttgtgatgaaggatttcaattaaaaggcagctctgctagttactgtg |
| | | tcttggctggaatggaaagcctttggaatagcagtgttccagtgtgtgaacaaatcttttgtccaagtcctccagttattccta |
| | | atgggagacacacaggaaaacctctggaagtctttccctttgggaaaacagtaaattacacatgcgacccccacccagac |
| | | agagggacgagcttcgacctcattggagagagcaccatccgctgcacaagtgaccctcaagggaatggggtttggagca |
| | | gccctgcccctcgctgtggaattctgggtcactgtcaagccccagatcattttctgtttgccaagttgaaaacccaaaccaat |
| | | gcatctgactttcccattgggacatctttaaagtacgaatgccgtcctgagtactacgggaggccattctctatcacatgtct |
| | | agataacctggtctggtcaagtcccaaagatgtctgtaaacgtaaatcatgtaaaactcctccagatccagtgaatggcat |
| | | ggtgcatgtgatcacagacatccaggttggatccagaatcaactattcttgtactacagggcaccgactcattggtcactca |
| | | tctgctgaatgtatcctctcgggcaatgctgcccattggagcacgaagccgccaatttgtcaacgaattccttgtgggctac |
| | | cccccaccatcgccaatggagatttcattagcaccaacagagagaattttcactatggatcagtggtgacctaccgctgca |
| | | atcctggaagcggagggagaaaggtgtttgagcttgtgggtgagccctccatatactgcaccagcaatgacgatcaagtg |
| | | ggcatctggagcggcccggcccctcagtgcattatacctaacaaatagcaattgctgtgccttctagttgccagccatctgttgt |
| | | ttgcccctcccccgtgccttccttgaccctggaaggtgccactcccactgtcctttcctaataaaatgaggaaattgcatcgcatt |
| | | gtctgagtaggtgtcattctattctggggggtggggggggcaggacagcaagggggaggattgggaagacaatagcagg |
| | | catgctggggatgcggtgggctctatg |
| 52 | CR2CR1 | ctagttattaatagtaatcaattacggggtcattagttcatagcccatatatggagttccgcgttacataacttacggtaaat |
| | #2B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtcatctggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtagatgtgtaattgctggacagggagttg |
| | | cttggaccaaaatgccagtatgtgaagaaatttttggtggaggtggatcaggaggtggtggatctcaatgcaatgccccagaat |
| | | ggcttccatttgccaggcctaccaacctaactgatgaatttgagtttcccattgggacatatctgaactatgaatgccgccct |
| | | ggttatagcggaagaccattttctatcatctgcctaaaaaactcagtctggactggtgctaaggacaggtgcagaaggaa |
| | | atcatgtagaaatcctccagatcctgtgaatggcatggtgcatgtgatcaaaggcatccagtttggatcccaaattaaatat |
| | | tcttgtactaaaggataccgcctcattggttcctcatctgccacatgcatcatctcaggtgatactgtcatttgggataatgaa |
| | | acacctatttgtgacagaattccttgtgggctaccccccaccatcaccaatggagatttcattagcaccaacagagagaatt |
| | | ttcactatggatcagtggtgacctaccgctgcaatcctggaagcggagggagaaaggtgtttgagcttgtgggtgagccct |
| | | ccatatactgcaccagcaatgatgatcaagtgggcatctggagcggccccgcccctcagtgcattatacctaacaaatgc |
| | | actcctccaaatgtggaaaatggaatattggtatctgacaacagaagcttattttccttaaatgaagttgtggagtttaggtg |
| | | tcagcctggctttgtcatgaaaggaccccgcagggtgaagtgccaggccctgaacaaatgggagccagagctaccaagc |
| | | tgctccagggtatgtcagccacctccagatgtcctgcatgctgagcgcacccaaagggacaaggacaacttttcacctgg |
| | | gcaggaagtgttctacagctgtgagcctggctatgacctcagaggggctgcatctatgcgctgcacaccccagggagact |
| | | ggagccctgcagcccccacatgtgaagtgaaatcctgtgatgacttcatgggccaacttcttaatggcagagtgctatttcc |
| | | agtaaatctccagcttggagcaaaagtggattttgtttgtgatgaaggatttcaattaaaaggcagctctgctagttactgtg |
| | | tcttggctggaatggaaagcctttggaatagcagtgttccagtgtgtgaacaaatcttttgtccaagtcctccagttattccta |
| | | atgggagacacacaggaaaacctctggaagtctttccctttgggaaaacagtaaattacacatgtgacccccacccagac |
| | | agagggactagctttgacctcattggagagagcaccatccgctgcacaagtgaccctcaagggaatggggtttggagca |
| | | gccctgcccctaggtgtggaattctgggtcactgtcaagccccagatcattttctgtttgccaagttgaaaacccaaaccaa |
| | | tgcatctgactttcccattgggacatctttaaagtatgaatgccgccctgagtactatgggaggccattctctatcacatgtct |
| | | agataacctggtctggtcaagtcccaaagatgtctgtaaaagaaaatcatgtaaaactcctccagatccagtgaatggcat |
| | | ggtgcatgtgatcacagacatccaggttggatccagaatcaactattcttgtacaactgggcaccgcctcattggtcactca |
| | | tctgctgaatgtatcctcagtggcaatgctgcccattggagcactaagccgccaatttgtcaaaggattccttgtgggctac |
| | | cccccaccattgccaatggagatttcattagcaccaacagagagaattttcactatggatcagtggtgacctaccgctgca |
| | | atcctggaagcggagggagaaaggtgtttgagcttgtgggtgagccctccatatactgcaccagcaatgatgatcaagtg |
| | | ggcatctggagcggcccagcccctcagtgcattatacctaacaaatagcaattgctgtgccttctagttgccagccatctgttgt |
| | | ttgcccctcccccgtgccttccttgaccctggaaggtgccactcccactgtcctttcctaataaaatgaggaaattgcatcgcatt |
| | | gtctgagtaggtgtcattctattctggggggtggggggggcaggacagcaagggggaggattgggaagacaatagcagg |
| | | catgctggggatgcggtgggctctatg |
| 53 | CR2CR1 | ctagttattaatagtaatcaattacggggtcattagttcatagcccatatatggagttccgcgttacataacttacggtaaat |
| | #3A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttcaatgcaatgccccagaatggcttccatttgccaggcctaccaaccta |
| | | actgatgaatttgagtttcccattgggacatatctgaactatgaatgccgccctggttattccggaagaccgttttctatcatc |
| | | tgcctaaaaaactcagtctggactggtgctaaggacaggtgcagacgtaaatcatgtcgtaatcctccagatcctgtgaat |
| | | ggcatggtgcatgtgatcaaaggcatccagttcggatcccaaattaaatattcttgtactaaaggataccgactcattggtt |
| | | cctcgtctgccacatgcatcatctcaggtgatactgtcatttgggataatgaaacacctatttgtgacagaattccttgtggg |
| | | ctaccccccaccatcaccaatggagatttcattagcaccaacagagagaattttcactatggatcagtggtgacctaccgc |
| | | tgcaatcctggaagcggagggagaaaggtgtttgagcttgtgggtgagccctccatatactgcaccagcaatgacgatca |
| | | agtgggcatctggagcggccccgcccctcagtgcataattggtcactgtcaagccccagatcattttctgtttgccaagttg |
| | | aaaacccaaaccaatgcatctgactttcccattgggacatctttaaagtacgaatgccgtcctgagtactacgggaggcca |
| | | ttctctatcacatgtctagataacctggtctggtcaagtcccaaagatgtctgtaaacgtaaatcatgtaaaactcctccaga |
| | | tccagtgaatggcatggtgcatgtgatcacagacatccaggttggatccagaatcaactattcttgtactacagggcaccg |
| | | actcattggtcactcatctgctgaatgtatcctctcgggcaatgctgcccattggagcacgaagccgccaatttgtcaacga |
| | | attccttgtgggctaccccccaccatcgccaatggagatttcattagcaccaacagagagaattttcactatggatcagtgg |
| | | tgacctaccgctgcaatcctggaagcggagggagaaaggtgtttgagcttgtgggtgagccctccatatactgcaccagc |
| | | aatgacgatcaagtgggcatctggagcggcccggcccctcagtgcattatacctaacaaaggtcactgtcaagccccaga |
| | | tcattttctgtttgccaagttgaaaacccaaaccaatgcatctgactttcccattgggacatctttaaagtacgaatgccgtc |
| | | ctgagtactacgggaggccattctctatcacatgtctagataacctggtctggtcaagtcccaaagatgtctgtaaacgtaa |
| | | atcatgtaaaactcctccagatccagtgaatggcatggtgcatgtgatcacagacatccaggttggatccagaatcaacta |
| | | ttcttgtactacagggcaccgactcattggtcactcatctgctgaatgtatcctctcaggcaatactgcccattggagcacga |
| | | agccgccaatttgtcaacgaattccttgtgggctacccccaaccatcgccaatggagatttcattagcaccaacagagaga |
| | | attttcactatggatcagtggtgacctaccgctgcaatcttggaagcagagggagaaaggtgtttgagcttgtgggtgagc |
| | | cctccatatactgcaccagcaatgacgatcaagtgggcatctggagcggccccgcccctcagtgcattatatagcaattgct |
| | | gtgccttctagttgccagccatctgttgtttgcccctcccccgtgccttccttgaccctggaaggtgccactcccactgtcctttccta |
| | | ataaaatgaggaaattgcatcgcattgtctgagtaggtgtcattctattctggggggtggggggggcaggacagcaagggg |
| | | gaggattgggaagacaatagcaggcatgctggggatgcggtgggctctatg |
| 54 | CR2CR1 | ctagttattaatagtaatcaattacggggtcattagttcatagcccatatatggagttccgcgttacataacttacggtaaat |
| | #3B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctaggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttcaatgcaatgccccagaatggcttccatttgccaggcctaccaacctaa |
| | | ctgatgaatttgagtttcccattgggacatatctgaactatgaatgccgccctggttatagcggaagaccattttctatcatct |
| | | gcctaaaaaactcagtctggactggtgctaaggacaggtgcagaaggaaatcatgtagaaatcctccagatcctgtgaat |
| | | ggcatggtgcatgtgatcaaaggcatccagtttggatcccaaattaaatattcttgtactaaaggataccgcctcattggttc |
| | | cagttctgccacatgcatcatctcaggtgatactgtcatttgggataatgaaacacctatttgtgacagaattccttgtgggc |
| | | taccccccaccatcaccaatggagatttcattagcaccaacagagagaattttcactatggatcagtggtgacctaccgct |
| | | gcaatcctggaagcggagggagaaaggtgtttgagcttgtgggtgagccctccatatactgcaccagcaatgatgatcaa |
| | | gtgggcatctggagcggccccgcccctcagtgcataattggtcactgtcaagccccagatcattttctgtttgccaagttga |
| | | aaacccaaaccaatgcatctgactttcccattgggacatctttaaagtatgaatgccgccctgagtactatgggaggccatt |
| | | ctctatcacatgtctagataacctggtctggtcaagtcccaaagatgtctgtaaaaggaaatcatgtaaaactcctccagat |
| | | ccagtgaatggcatggtgcatgtgatcacagacatccaggttggatccagaatcaactattcttgtacaactgggcaccgc |
| | | ctcattggtcactcatctgctgaatgtatcctcagtggcaatgctgcccattggagcactaagccgccaatttgtcaaagaa |
| | | ttccttgtgggctaccccccaccattgccaatggagatttcattagcaccaacagagagaattttcactatggatcagtggt |
| | | gacctaccgctgcaatcctggaagcggagggagaaaggtgtttgagcttgtgggtgagccctccatatactgcaccagca |
| | | atgatgatcaagtgggcatctggagcggcccagcccctcagtgcattatacctaacaaaggtcactgtcaagccccagat |
| | | cattttctgtttgccaagttgaaaacccaaaccaatgcatctgactttcccattgggacatctttaaagtatgaatgccgccc |
| | | tgagtactatgggaggccattctctatcacatgtctagataacctggtctggtcaagtcccaaagatgtctgtaaaaggaa |
| | | atcatgtaaaactcctccagatccagtgaatggcatggtgcatgtgatcacagacatccaggttggatccagaatcaacta |
| | | ttcttgtacaactgggcaccgcctcattggtcactcatctgctgaatgtatcctctcaggcaatactgcccattggagcacta |
| | | agccgccaatttgtcaaagaattccttgtgggctacccccaaccattgccaatggagatttcattagcaccaacagagaga |
| | | attttcactatggatcagtggtgacctaccgctgcaatcttggaagcagagggagaaaggtgtttgagcttgtgggtgagc |
| | | cctccatatactgcaccagcaatgatgatcaagtgggcatctggagcggccccgcccctcagtgcattatatagcaattgct |
| | | gtgccttctagttgccagccatctgttgtttgcccctcccccgtgccttccttgaccctggaaggtgccactcccactgtcctttccta |
| | | ataaaatgaggaaattgcatcgcattgtctgagtaggtgtcattctattctggggggtggggggggcaggacagcaagggg |
| | | gaggattgggaagacaatagcaggcatgctggggatgcggtgggctctatg |
| 55 | CR2CR1 | ctagttattaatagtaatcaattacggggtcattagttcatagcccatatatggagttccgcgttacataacttacggtaaat |
| | #4A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacgactaccaacct |
| | | gtgtaagtgttttccctctcgagtgtccagcacttcctatgatccacaatggacatcacacaagtgagaatggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttggtggaggtggatcgggaggtggtggatctcaatgcaatgccccagaa |
| | | tggcttccatttgccaggcctaccaacctaactgatgaatttgagtttcccattgggacatatctgaactatgaatgccgccc |
| | | tggttattccggaagaccgttttctatcatctgcctaaaaaactcagtctggactggtgctaaggacaggtgcagacgtaaa |
| | | tcatgtcgtaatcctccagatcctgtgaatggcatggtgcatgtgatcaaaggcatccagttcggatcccaaattaaatatt |
| | | cttgtactaaaggataccgactcattggttcctcgtctgccacatgcatcatctcaggtgatactgtcatttgggataatgaa |
| | | acacctatttgtgacagaattccttgtgggctaccccccaccatcaccaatggagatttcattagcaccaacagagagaatt |
| | | ttcactatggatcagtggtgacctaccgctgcaatcctggaagcggagggagaaaggtgtttgagcttgtgggtgagccct |
| | | ccatatactgcaccagcaatgacgatcaagtgggcatctggagcggccccgcccctcagtgcataattggtcactgtcaa |
| | | gccccagatcattttctgtttgccaagttgaaaacccaaaccaatgcatctgactttcccattgggacatctttaaagtacga |
| | | atgccgtcctgagtactacgggaggccattctctatcacatgtctagataacctggtctggtcaagtcccaaagatgtctgt |
| | | aaacgtaaatcatgtaaaactcctccagatccagtgaatggcatggtgcatgtgatcacagacatccaggttggatccag |
| | | aatcaactattcttgtactacagggcaccgactcattggtcactcatctgctgaatgtatcctctcgggcaatgctgcccatt |
| | | ggagcacgaagccgccaatttgtcaacgaattccttgtgggctaccccccaccatcgccaatggagatttcattagcacca |
| | | acagagagaattttcactatggatcagtggtgacctaccgctgcaatcctggaagcggagggagaaaggtgtttgagctt |
| | | gtgggtgagccctccatatactgcaccagcaatgacgatcaagtgggcatctggagcggcccggcccctcagtgcattat |
| | | acctaacaaaggtcactgtcaagccccagatcattttctgtttgccaagttgaaaacccaaaccaatgcatctgactttccc |
| | | attgggacatctttaaagtacgaatgccgtcctgagtactacgggaggccattctctatcacatgtctagataacctggtct |
| | | ggtcaagtcccaaagatgtctgtaaacgtaaatcatgtaaaactcctccagatccagtgaatggcatggtgcatgtgatca |
| | | cagacatccaggttggatccagaatcaactattcttgtactacagggcaccgactcattggtcactcatctgctgaatgtat |
| | | cctctcaggcaatactgcccattggagcacgaagccgccaatttgtcaacgaattccttgtgggctacccccaaccatcgc |
| | | caatggagatttcattagcaccaacagagagaattttcactatggatcagtggtgacctaccgctgcaatcttggaagcag |
| | | agggagaaaggtgtttgagcttgtgggtgagccctccatatactgcaccagcaatgacgatcaagtgggcatctggagcg |
| | | gccccgcccctcagtgcattatatagcaattgctgtgccttctagttgccagccatctgttgtttgcccctcccccgtgccttccttg |
| | | accctggaaggtgccactcccactgtcctttcctaataaaatgaggaaattgcatcgcattgtctgagtaggtgtcattctattctg |
| | | gggggtggggggggcaggacagcaagggggaggattgggaagacaatagcaggcatgctggggatgcggtgggctct |
| | | atg |
| 56 | CR2CR1 | ctagttattaatagtaatcaattacggggtcattagttcatagcccatatatggagttccgcgttacataacttacggtaaat |
| | #4B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtctagtggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttggtggaggtggaagtggaggtggtggatctcaatgcaatgccccagaa |
| | | tggcttccatttgccaggcctaccaacctaactgatgaatttgagtttcccattgggacatatctgaactatgaatgccgccc |
| | | tggttatagcggaagaccattttctatcatctgcctaaaaaactcagtctggactggtgctaaggacaggtgcagaaggaa |
| | | atcatgtagaaatcctccagatcctgtgaatggcatggtgcatgtgatcaaaggcatccagtttggatcccaaattaaatat |
| | | tcttgtactaaaggataccgcctcattggttccagttctgccacatgcatcatctcaggtgatactgtcatttgggataatga |
| | | aacacctatttgtgacagaattccttgtgggctaccccccaccatcaccaatggagatttcattagcaccaacagagagaa |
| | | ttttcactatggatcagtggtgacctaccgctgcaatcctggaagcggagggagaaaggtgtttgagcttgtgggtgagcc |
| | | ctccatatactgcaccagcaatgatgatcaagtgggcatctggagcggccccgcccctcagtgcataattggtcactgtca |
| | | agccccagatcattttctgtttgccaagttgaaaacccaaaccaatgcatctgactttcccattgggacatctttaaagtatg |
| | | aatgccgccctgagtactatgggaggccattctctatcacatgtctagataacctggtctggtcaagtcccaaagatgtctg |
| | | taaaaggaaatcatgtaaaactcctccagatccagtgaatggcatggtgcatgtgatcacagacatccaggttggatcca |
| | | gaatcaactattcttgtacaactgggcaccgcctcattggtcactcatctgctgaatgtatcctcagtggcaatgctgcccat |
| | | tggagcactaagccgccaatttgtcaaagaattccttgtgggctaccccccaccattgccaatggagatttcattagcacca |
| | | acagagagaattttcactatggatcagtggtgacctaccgctgcaatcctggaagcggagggagaaaggtgtttgagctt |
| | | gtgggtgagccctccatatactgcaccagcaatgatgatcaagtgggcatctggagcggcccagcccctcagtgcattata |
| | | cctaacaaaggtcactgtcaagccccagatcattttctgtttgccaagttgaaaacccaaaccaatgcatctgactttccca |
| | | ttgggacatctttaaagtatgaatgccgccctgagtactatgggaggccattctctatcacatgtctagataacctggtctgg |
| | | tcaagtcccaaagatgtctgtaaaaggaaatcatgtaaaactcctccagatccagtgaatggcatggtgcatgtgatcaca |
| | | gacatccaggttggatccagaatcaactattcttgtacaactgggcaccgcctcattggtcactcatctgctgaatgtatcct |
| | | ctcaggcaatactgcccattggagcactaagccgccaatttgtcaaagaattccttgtgggctacccccaaccattgccaat |
| | | ggagatttcattagcaccaacagagagaattttcactatggatcagtggtgacctaccgctgcaatcttggaagcagagg |
| | | gagaaaggtgtttgagcttgtgggtgagccctccatatactgcaccagcaatgatgatcaagtgggcatctggagcggcc |
| | | ccgcccctcagtgcattatatagcaattgctgtgccttctagttgccagccatctgttgtttgcccctcccccgtgccttccttgacc |
| | | ctggaaggtgccactcccactgtcctttcctaataaaatgaggaaattgcatcgcattgtctgagtaggtgtcattctattctggg |
| | | gggtggggggggcaggacagcaagggggaggattgggaagacaatagcaggcatgctggggatgcggtgggctctatg |
| 57 | CR2CR1 | ctagttattaatagtaatcaattacggggtcattagttcatagcccatatatggagttccgcgttacataacttacggtaaat |
| | #5A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttcaatgcaatgccccagaatggcttccatttgccaggcctaccaaccta |
| | | actgatgaatttgagtttcccattgggacatatctgaactatgaatgccgccctggttattccggaagaccgttttctatcatc |
| | | tgcctaaaaaactcagtctggactggtgctaaggacaggtgcagacgtaaatcatgtcgtaatcctccagatcctgtgaat |
| | | ggcatggtgcatgtgatcaaaggcatccagttcggatcccaaattaaatattcttgtactaaaggataccgactcattggtt |
| | | cctcgtctgccacatgcatcatctcaggtgatactgtcatttgggataatgaaacacctatttgtgacagaattccttgtggg |
| | | ctaccccccaccatcaccaatggagatttcattagcaccaacagagagaattttcactatggatcagtggtgacctaccgc |
| | | tgcaatcctggaagcggagggagaaaggtgtttgagcttgtgggtgagccctccatatactgcaccagcaatgacgatca |
| | | agtgggcatctggagcggccccgcccctcagtgcattatacctaacaaatgcacgcctccaaatgtggaaaatggaatat |
| | | tggtatctgacaacagaagcttattttccttaaatgaagttgtggagtttaggtgtcagcctggctttgtcatgaaaggaccc |
| | | cgccgtgtgaagtgccaggccctgaacaaatgggagccggagctaccaagctgctccagggtatgtcagccacctccag |
| | | atgtcctgcatgctgagcgtacccaaagggacaaggacaacttttcacctgggcaggaagtgttctacagctgtgagccc |
| | | ggctacgacctcagaggggctgcgtctatgcgctgcacaccccagggagactggagccctgcagcccccacatgtgaag |
| | | tgaaatcctgtgatgacttcatgggccaacttcttaatggccgtgtgctatttccagtaaatctccagcttggagcaaaagtg |
| | | gattttgtttgtgatgaaggatttcaattaaaaggcagctctgctagttactgtgtcttggctggaatggaaagcctttggaa |
| | | tagcagtgttccagtgtgtgaacaaatcttttgtccaagtcctccagttattcctaatgggagacacacaggaaaacctctg |
| | | gaagtctttccctttgggaaaacagtaaattacacatgcgacccccacccagacagagggacgagcttcgacctcattgg |
| | | agagagcaccatccgctgcacaagtgaccctcaagggaatggggtttggagcagccctgcccctcgctgtggaattctgg |
| | | gtcactgtcaagccccagatcattttctgtttgccaagttgaaaacccaaaccaatgcatctgactttcccattgggacatct |
| | | ttaaagtacgaatgccgtcctgagtactacgggaggccattctctatcacatgtctagataacctggtctggtcaagtccca |
| | | aagatgtctgtaaacgtaaatcatgtaaaactcctccagatccagtgaatggcatggtgcatgtgatcacagacatccagg |
| | | ttggatccagaatcaactattcttgtactacagggcaccgactcattggtcactcatctgctgaatgtatcctctcgggcaat |
| | | gctgcccattggagcacgaagccgccaatttgtcaacgaattccttgtgggctaccccccaccatcgccaatggagatttc |
| | | attagcaccaacagagagaattttcactatggatcagtggtgacctaccgctgcaatcctggaagcggagggagaaagg |
| | | tgtttgagcttgtgggtgagccctccatatactgcaccagcaatgacgatcaagtgggcatctggagcggcccggcccctc |
| | | agtgcattatacctaacaaatgcacgcctccaaatgtggaaaatggaatattggtatctgacaacagaagcttattttcctt |
| | | aaatgaagttgtggagtttaggtgtcagcctggctttgtcatgaaaggaccccgccgtgtgaagtgccaggccctgaacaa |
| | | atgggagccggagctaccaagctgctccagggtatgtcagccacctccagatgtcctgcatgctgagcgtacccaaaggg |
| | | acaaggacaacttttcacccgggcaggaagtgttctacagctgtgagcccggctatgacctcagaggggctgcgtctatg |
| | | cgctgcacaccccagggagactggagccctgcagcccccacatgtgaagtgaaatcctgtgatgacttcatgggccaact |
| | | tcttaatggccgtgtgctatttccagtaaatctccagcttggagcaaaagtggattttgtttgtgatgaaggatttcaattaaa |
| | | aggcagctctgctagttattgtgtcttggctggaatggaaagcctttggaatagcagtgttccagtgtgtgaacaaatctttt |
| | | gtccaagtcctccagttattcctaatgggagacacacaggaaaacctctggaagtctttccctttggaaaagcagtaaatt |
| | | acacatgcgacccccacccagacagagggacgagcttcgacctcattggagagagcaccatccgctgcacaagtgaccc |
| | | tcaagggaatggggtttggagcagccctgcccctcgctgtggaatttagcaattgctgtgccttctagttgccagccatctgttg |
| | | tttgcccctcccccgtgccttccttgaccctggaaggtgccactcccactgtcctttcctaataaaatgaggaaattgcatcgcatt |
| | | gtctgagtaggtgtcattctattctggggggtggggggggcaggacagcaagggggaggattgggaagacaatagcagg |
| | | catgctggggatgcggtgggctctatg |
| 58 | CR2CR1 | ctagttattaatagtaatcaattacggggtcattagttcatagcccatatatggagttccgcgttacataacttacggtaaat |
| | #5B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtcatctggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttcaatgcaatgccccagaatggcttccatttgccaggcctaccaacctaa |
| | | ctgatgaatttgagtttcccattgggacatatctgaactatgaatgccgccctggttatagcggaagaccattttctatcatct |
| | | gcctaaaaaactcagtctggactggtgctaaggacaggtgcagaagaaaatcatgtaggaatcctccagatcctgtgaat |
| | | ggcatggtgcatgtgatcaaaggcatccagtttggatcccaaattaaatattcttgtactaaaggataccgcctcattggttc |
| | | ctcatctgccacatgcatcatctcaggtgatactgtcatttgggataatgaaacacctatttgtgacagaattccttgtgggc |
| | | taccccccaccatcaccaatggagatttcattagcaccaacagagagaattttcactatggatcagtggtgacctaccgct |
| | | gcaatcctggaagcggagggagaaaggtgtttgagcttgtgggtgagccctccatatactgcaccagcaatgatgatcaa |
| | | gtgggcatctggagcggccccgcccctcagtgcattatacctaacaaatgcactcctccaaatgtggaaaatggaatattg |
| | | gtatctgacaacagaagcttattttccttaaatgaagttgtggagtttaggtgtcagcctggctttgtcatgaaaggaccccg |
| | | cagagtgaagtgccaggccctgaacaaatgggagccagagctaccaagctgctccagggtatgtcagccacctccagat |
| | | gtcctgcatgctgagcgcacccaaagggacaaggacaacttttcacctgggcaggaagtgttctacagctgtgagcctgg |
| | | ctatgacctcagaggggctgcatctatgcgctgcacaccccagggagactggagccctgcagcccccacatgtgaagtga |
| | | aatcctgtgatgacttcatgggccaacttcttaatggcagggtgctatttccagtaaatctccagcttggagcaaaagtgga |
| | | ttttgtttgtgatgaaggatttcaattaaaaggcagctctgctagttactgtgtcttggctggaatggaaagcctttggaata |
| | | gcagtgttccagtgtgtgaacaaatcttttgtccaagtcctccagttattcctaatgggagacacacaggaaaacctctgga |
| | | agtctttccctttgggaaaacagtaaattacacatgtgacccccacccagacagagggactagctttgacctcattggaga |
| | | gagcaccatccgctgcacaagtgaccctcaagggaatggggtttggagcagccctgcccctagatgtggaattctgggtc |
| | | actgtcaagccccagatcattttctgtttgccaagttgaaaacccaaaccaatgcatctgactttcccattgggacatcttta |
| | | aagtatgaatgccgccctgagtactatgggaggccattctctatcacatgtctagataacctggtctggtcaagtcccaaag |
| | | atgtctgtaaaaggaaatcatgtaaaactcctccagatccagtgaatggcatggtgcatgtgatcacagacatccaggttg |
| | | gatccagaatcaactattcttgtacaactgggcaccgcctcattggtcactcatctgctgaatgtatcctctcaggcaatgct |
| | | gcccattggagcactaagccgccaatttgtcaaagaattccttgtgggctaccccccaccattgccaatggagatttcatta |
| | | gcaccaacagagagaattttcactatggatcagtggtgacctaccgctgcaatcctggaagcggagggagaaaggtgttt |
| | | gagcttgtgggtgagccctccatatactgcaccagcaatgatgatcaagtgggcatctggagcggcccagcccctcagtg |
| | | cattatacctaacaaatgcactcctccaaatgtggaaaatggaatattggtatctgacaacagaagcttattttccttaaat |
| | | gaagttgtggagtttaggtgtcagcctggctttgtcatgaaaggaccccgcagggtgaagtgccaggccctgaacaaatg |
| | | ggagccagagctaccaagctgctccagggtatgtcagccacctccagatgtcctgcatgctgagcgcacccaaagggac |
| | | aaggacaacttttcacctgggcaggaagtgttctacagctgtgagcctggctatgacctcagaggggctgcatctatgcgc |
| | | tgcacaccccagggagactggagccctgcagcccccacatgtgaagtgaaatcctgtgatgacttcatgggccaacttctt |
| | | aatggcagagtgctatttccagtaaatctccagcttggagcaaaagtggattttgtttgtgatgaaggatttcaattaaaag |
| | | gcagctctgctagttattgtgtcttggctggaatggaaagcctttggaatagcagtgttccagtgtgtgaacaaatcttttgtc |
| | | caagtcctccagttattcctaatgggagacacacaggaaaacctctggaagtctttccctttggaaaagcagtaaattaca |
| | | catgtgacccccacccagacagagggactagctttgacctcattggagagagcaccatccgctgcacaagtgaccctcaa |
| | | gggaatggggtttggagcagccctgcccctaggtgtggaatttagcaattgctgtgccttctagttgccagccatctgttgtttg |
| | | cccctcccccgtgccttccttgaccctggaaggtgccactcccactgtcctttcctaataaaatgaggaaattgcatcgcattgtc |
| | | tgagtaggtgtcattctattctggggggtggggggggcaggacagcaagggggaggattgggaagacaatagcaggcat |
| | | gctggggatgcggtgggctctatg |
| 59 | CR2CR1 | ctagttattaatagtaatcaattacggggtcattagttcatagcccatatatggagttccgcgttacataacttacggtaaat |
| | #6A | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttggtggaggtggatcgggaggtggtggatctcaatgcaatgccccagaa |
| | | tggcttccatttgccaggcctaccaacctaactgatgaatttgagtttcccattgggacatatctgaactatgaatgccgccc |
| | | tggttattccggaagaccgttttctatcatctgcctaaaaaactcagtctggactggtgctaaggacaggtgcagacgtaaa |
| | | tcatgtcgtaatcctccagatcctgtgaatggcatggtgcatgtgatcaaaggcatccagttcggatcccaaattaaatatt |
| | | cttgtactaaaggataccgactcattggttcctcgtctgccacatgcatcatctcaggtgatactgtcatttgggataatgaa |
| | | acacctatttgtgacagaattccttgtgggctaccccccaccatcaccaatggagatttcattagcaccaacagagagaatt |
| | | ttcactatggatcagtggtgacctaccgctgcaatcctggaagcggagggagaaaggtgtttgagcttgtgggtgagccct |
| | | ccatatactgcaccagcaatgacgatcaagtgggcatctggagcggccccgcccctcagtgcattatacctaacaaatgc |
| | | acgcctccaaatgtggaaaatggaatattggtatctgacaacagaagcttattttccttaaatgaagttgtggagtttaggt |
| | | gtcagcctggctttgtcatgaaaggaccccgccgtgtgaagtgccaggccctgaacaaatgggagccggagctaccaag |
| | | ctgctccagggtatgtcagccacctccagatgtcctgcatgctgagcgtacccaaagggacaaggacaacttttcacctgg |
| | | gcaggaagtgttctacagctgtgagcccggctacgacctcagaggggctgcgtctatgcgctgcacaccccagggagact |
| | | ggagccctgcagcccccacatgtgaagtgaaatcctgtgatgacttcatgggccaacttcttaatggccgtgtgctatttcc |
| | | agtaaatctccagcttggagcaaaagtggattttgtttgtgatgaaggatttcaattaaaaggcagctctgctagttactgtg |
| | | tcttggctggaatggaaagcctttggaatagcagtgttccagtgtgtgaacaaatcttttgtccaagtcctccagttattccta |
| | | atgggagacacacaggaaaacctctggaagtctttccctttgggaaaacagtaaattacacatgcgacccccacccagac |
| | | agagggacgagcttcgacctcattggagagagcaccatccgctgcacaagtgaccctcaagggaatggggtttggagca |
| | | gccctgcccctcgctgtggaattctgggtcactgtcaagccccagatcattttctgtttgccaagttgaaaacccaaaccaat |
| | | gcatctgactttcccattgggacatctttaaagtacgaatgccgtcctgagtactacgggaggccattctctatcacatgtct |
| | | agataacctggtctggtcaagtcccaaagatgtctgtaaacgtaaatcatgtaaaactcctccagatccagtgaatggcat |
| | | ggtgcatgtgatcacagacatccaggttggatccagaatcaactattcttgtactacagggcaccgactcattggtcactca |
| | | tctgctgaatgtatcctctcgggcaatgctgcccattggagcacgaagccgccaatttgtcaacgaattccttgtgggctac |
| | | cccccaccatcgccaatggagatttcattagcaccaacagagagaattttcactatggatcagtggtgacctaccgctgca |
| | | atcctggaagcggagggagaaaggtgtttgagcttgtgggtgagccctccatatactgcaccagcaatgacgatcaagtg |
| | | ggcatctggagcggcccggcccctcagtgcattatacctaacaaatgcacgcctccaaatgtggaaaatggaatattggt |
| | | atctgacaacagaagcttattttccttaaatgaagttgtggagtttaggtgtcagcctggctttgtcatgaaaggaccccgcc |
| | | gtgtgaagtgccaggccctgaacaaatgggagccggagctaccaagctgctccagggtatgtcagccacctccagatgtc |
| | | ctgcatgctgagcgtacccaaagggacaaggacaacttttcacccgggcaggaagtgttctacagctgtgagcccggcta |
| | | tgacctcagaggggctgcgtctatgcgctgcacaccccagggagactggagccctgcagcccccacatgtgaagtgaaa |
| | | tcctgtgatgacttcatgggccaacttcttaatggccgtgtgctatttccagtaaatctccagcttggagcaaaagtggatttt |
| | | gtttgtgatgaaggatttcaattaaaaggcagctctgctagttattgtgtcttggctggaatggaaagcctttggaatagca |
| | | gtgttccagtgtgtgaacaaatcttttgtccaagtcctccagttattcctaatgggagacacacaggaaaacctctggaagt |
| | | ctttccctttggaaaagcagtaaattacacatgcgacccccacccagacagagggacgagcttcgacctcattggagaga |
| | | gcaccatccgctgcacaagtgaccctcaagggaatggggtttggagcagccctgcccctcgctgtggaatttagcaattgct |
| | | gtgccttctagttgccagccatctgttgtttgcccctcccccgtgccttccttgaccctggaaggtgccactcccactgtcctttccta |
| | | ataaaatgaggaaattgcatcgcattgtctgagtaggtgtcattctattctggggggtggggggggcaggacagcaagggg |
| | | gaggattgggaagacaatagcaggcatgctggggatgcggtgggctctatg |
| 60 | CR2CR1 | ctagttattaatagtaatcaattacggggtcattagttcatagcccatatatggagttccgcgttacataacttacggtaaat |
| | #6B | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaatggaaacaagtctgtttggtgtcaagcaaataatatgtgggggccaacaaggctaccaacctg |
| | | tgtaagtgttttccctttggagtgtccagcacttcctatgatccacaatggacatcacacaagtgagaatgttggctccattgctccag |
| | | gattgtctgtgacttacagctgtgaatctggttacttgcttgttggagaaaagatcattaactgtttgtcatctggaaaatggagtgctgt |
| | | cccccccacatgtgaagaggcaaggtgtaaatctctaggaaggtttcccaatgggaaggtaaaggagcctccaattctcagggtt |
| | | ggtgtaactgcaaactttttctgtgatgaagggtataggctgcaaggcccaccttctagtaggtgtgtaattgctggacagggagttg |
| | | cttggaccaaaatgccagtatgtgaagaaatttttggtggaggtggatcaggaggtggtggatctcaatgcaatgccccagaat |
| | | ggcttccatttgccaggcctaccaacctaactgatgaatttgagtttcccattgggacatatctgaactatgaatgccgccct |
| | | ggttatagcggaagaccattttctatcatctgcctaaaaaactcagtctggactggtgctaaggacaggtgcagaagaaa |
| | | atcatgtaggaatcctccagatcctgtgaatggcatggtgcatgtgatcaaaggcatccagtttggatcccaaattaaatat |
| | | tcttgtactaaaggataccgcctcattggttcctcatctgccacatgcatcatctcaggtgatactgtcatttgggataatgaa |
| | | acacctatttgtgacagaattccttgtgggctaccccccaccatcaccaatggagatttcattagcaccaacagagagaatt |
| | | ttcactatggatcagtggtgacctaccgctgcaatcctggaagcggagggagaaaggtgtttgagcttgtgggtgagccct |
| | | ccatatactgcaccagcaatgatgatcaagtgggcatctggagcggccccgcccctcagtgcattatacctaacaaatgc |
| | | actcctccaaatgtggaaaatggaatattggtatctgacaacagaagcttattttccttaaatgaagttgtggagtttaggtg |
| | | tcagcctggctttgtcatgaaaggaccccgcagagtgaagtgccaggccctgaacaaatgggagccagagctaccaagc |
| | | tgctccagggtatgtcagccacctccagatgtcctgcatgctgagcgcacccaaagggacaaggacaacttttcacctgg |
| | | gcaggaagtgttctacagctgtgagcctggctatgacctcagaggggctgcatctatgcgctgcacaccccagggagact |
| | | ggagccctgcagcccccacatgtgaagtgaaatcctgtgatgacttcatgggccaacttcttaatggcagggtgctatttcc |
| | | agtaaatctccagcttggagcaaaagtggattttgtttgtgatgaaggatttcaattaaaaggcagctctgctagttactgtg |
| | | tcttggctggaatggaaagcctttggaatagcagtgttccagtgtgtgaacaaatcttttgtccaagtcctccagttattccta |
| | | atgggagacacacaggaaaacctctggaagtctttccctttgggaaaacagtaaattacacatgtgacccccacccagac |
| | | agagggactagctttgacctcattggagagagcaccatccgctgcacaagtgaccctcaagggaatggggtttggagca |
| | | gccctgcccctagatgtggaattctgggtcactgtcaagccccagatcattttctgtttgccaagttgaaaacccaaaccaa |
| | | tgcatctgactttcccattgggacatctttaaagtatgaatgccgccctgagtactatgggaggccattctctatcacatgtct |
| | | agataacctggtctggtcaagtcccaaagatgtctgtaaaaggaaatcatgtaaaactcctccagatccagtgaatggcat |
| | | ggtgcatgtgatcacagacatccaggttggatccagaatcaactattcttgtacaactgggcaccgcctcattggtcactca |
| | | tctgctgaatgtatcctctcaggcaatgctgcccattggagcactaagccgccaatttgtcaaagaattccttgtgggctacc |
| | | ccccaccattgccaatggagatttcattagcaccaacagagagaattttcactatggatcagtggtgacctaccgctgcaa |
| | | tcctggaagcggagggagaaaggtgtttgagcttgtgggtgagccctccatatactgcaccagcaatgatgatcaagtgg |
| | | gcatctggagcggcccagcccctcagtgcattatacctaacaaatgcactcctccaaatgtggaaaatggaatattggtat |
| | | ctgacaacagaagcttattttccttaaatgaagttgtggagtttaggtgtcagcctggctttgtcatgaaaggaccccgcag |
| | | ggtgaagtgccaggccctgaacaaatgggagccagagctaccaagctgctccagggtatgtcagccacctccagatgtc |
| | | ctgcatgctgagcgcacccaaagggacaaggacaacttttcacctgggcaggaagtgttctacagctgtgagcctggcta |
| | | tgacctcagaggggctgcatctatgcgctgcacaccccagggagactggagccctgcagcccccacatgtgaagtgaaa |
| | | tcctgtgatgacttcatgggccaacttcttaatggcagagtgctatttccagtaaatctccagcttggagcaaaagtggattt |
| | | tgtttgtgatgaaggatttcaattaaaaggcagctctgctagttattgtgtcttggctggaatggaaagcctttggaatagca |
| | | gtgttccagtgtgtgaacaaatcttttgtccaagtcctccagttattcctaatgggagacacacaggaaaacctctggaagt |
| | | ctttccctttggaaaagcagtaaattacacatgtgacccccacccagacagagggactagctttgacctcattggagagag |
| | | caccatccgctgcacaagtgaccctcaagggaatggggtttggagcagccctgcccctaggtgtggaatttagcaattgct |
| | | gtgccttctagttgccagccatctgttgtttgcccctcccccgtgccttccttgaccctggaaggtgccactcccactgtcctttccta |
| | | ataaaatgaggaaattgcatcgcattgtctgagtaggtgtcattctattctggggggtggggggggcaggacagcaagggg |
| | | gaggattgggaagacaatagcaggcatgctggggatgcggtgggctctatg |
| 61 | CR2CR1 | ctagttattaatagtaatcaattacggggtcattagttcatagcccatatatggagttccgcgttacataacttacggtaaat |
| | #13 | ggcccgcctggctgaccgcccaacgacccccgcccattgacgtcaataatgacgtatgttcccatagtaacgccaatagg |
| | | gactttccattgacgtcaatgggtggactatttacggtaaactgcccacttggcagtacatcaagtgtatcatatgccaagt |
| | | acgccccctattgacgtcaatgacggtaaatggcccgcctggcattatgcccagtacatgaccttatgggactttcctactt |
| | | ggcagtacatctacgtattagtcatcgctattaccatggtcgaggtgagccccacgttctgcttcactctccccatctccccc |
| | | ccctccccacccccaattttgtatttatttattttttaattattttgtgcagcgatgggggcggggggggggggggggcgcgc |
| | | gccaggcggggcggggcggggcgaggggcggggcggggcgaggcggagaggtgcggcggcagccaatcagagcgg |
| | | cgcgctccgaaagtttccttttatggcgaggcggcggcggcggcggccctataaaaagcgaagcgcgcgggggggg |
| | | agtcgctgcgacgctgccttcgccccgtgccccgctccgccgccgcctcgcgccgcccgccccggctctgactgaccgcgt |
| | | tactcccacaggtgagcgggcgggacggcccttctcctccgggctgtaattagcgcttggtttaatgacggcttgtttcttttc |
| | | tgtggctgcgtgaaagccttgaggggctccgggagctagagcctctgctaaccatgttcatgccttcttctttttcctacagct |
| | | cctgggcaacgtgctggttattgtgctgtctcatcattttggcaaagcggccgcaagcttgaattcatttaaatccagctagc |
| | | gccaccatgaggctcctggctttcctgagtctgctggccttggtgctgcaggagacagggacaatttcttgtggctctcctccgcc |
| | | tatcctaaatggcaggattagttattattctacccccattgctgttggtacagtgataaggtacagttgttcaggtaccttccgcctcatt |
| | | ggagaaaaaagtctattatgcataactaaagacaaagtggatggaacctgggataaacctgctcctaaatgtgaatatttcaataaat |
| | | attcttcttgccctgagcccatagtaccaggaggatacaaaattagaggctctacaccctacagacatggtgattctgtgacatttgc |
| | | ctgtaaaaccaacttctccatgaacggaaacaagtctgtttggtgtcaagcaaataatatgtgggggccgacacgactaccaacct |
| | | gtgtaagtgttttccctcgagtgtccagcacttcctatgatccacaatggacatcacacaagtgagaatgttggctccattgctcca |
| | | ggattgtctgtgacttacagctgtgaatctggttacttgcttgttggagaaaagatcattaactgtttgtcttcgggaaaatggagtgct |
| | | gtcccccccacatgtgaagaggcacgctgtaaatctctaggacgatttcccaatgggaaggtaaaggagcctccaattctccggg |
| | | ttggtgtaactgcaaactttttctgtgatgaagggtatcgactgcaaggcccaccttctagtcggtgtgtaattgctggacagggagtt |
| | | gcttggaccaaaatgccagtatgtgaagaaatttttcaatgcaatgccccagaatggcttccatttgccaggcctaccaaccta |
| | | actgatgaatttgagtttcccattgggacatatctgaactatgaatgccgccctggttattccggaagaccgttttctatcatc |
| | | tgcctaaaaaactcagtctggactggtgctaaggacaggtgcagacgtaaatcatgtcgtaatcctccagatcctgtgaat |
| | | ggcatggtgcatgtgatcaaaggcatccagttcggatcccaaattaaatattcttgtactaaaggataccgactcattggtt |
| | | cctcgtctgccacatgcatcatctcaggtgatactgtcatttgggataatgaaacacctatttgtgacagaattccttgtggg |
| | | ctaccccccaccatcaccaatggagatttcattagcaccaacagagagaattttcactatggatcagtggtgacctaccgc |
| | | tgcaatcctggaagcggagggagaaaggtgtttgagcttgtgggtgagccctccatatactgcaccagcaatgacgatca |
| | | agtgggcatctggagcggccccgcccctcagtgcattatacctaacaaatgcacgcctccaaatgtggaaaatggaatat |
| | | tggtatctgacaacagaagcttattttccttaaatgaagttgtggagtttaggtgtcagcctggctttgtcatgaaaggaccc |
| | | cgccgtgtgaagtgccaggccctgaacaaatgggagccggagctaccaagctgctccagggtatgtcagccacctccag |
| | | atgtcctgcatgctgagcgtacccaaagggacaaggacaacttttcacctgggcaggaagtgttctacagctgtgagccc |
| | | ggctacgacctcagaggggctgcgtctatgcgctgcacaccccagggagactggagccctgcagcccccacatgtgaag |
| | | tgaaatcctgtgatgacttcatgggccaacttcttaatggccgtgtgctatttccagtaaatctccagcttggagcaaaagtg |
| | | gattttgtttgtgatgaaggatttcaattaaaaggcagctctgctagttactgtgtcttggctggaatggaaagcctttggaa |
| | | tagcagtgttccagtgtgtgaacaaatcttttgtccaagtcctccagttattcctaatgggagacacacaggaaaacctctg |
| | | gaagtctttccctttgggaaaacagtaaattacacatgcgacccccacccagacagagggacgagcttcgacctcattgg |
| | | agagagcaccatccgctgcacaagtgaccctcaagggaatggggtttggagcagccctgcccctcgctgtggaattctgg |
| | | gtcactgtcaagccccagatcattttctgtttgccaagttgaaaacccaaaccaatgcatctgactttcccattgggacatct |
| | | ttaaagtacgaatgccgtcctgagtactacgggaggccattctctatcacatgtctagataacctggtctggtcaagtccca |
| | | aagatgtctgtaaacgtaaatcatgtaaaactcctccagatccagtgaatggcatggtgcatgtgatcacagacatccagg |
| | | ttggatccagaatcaactattcttgtactacagggcaccgactcattggtcactcatctgctgaatgtatcctctcgggcaat |
| | | gctgcccattggagcacgaagccgccaatttgtcaacgaattccttgtgggctaccccccaccatcgccaatggagatttc |
| | | attagcaccaacagagagaattttcactatggatcagtggtgacctaccgctgcaatcctggaagcggagggagaaagg |
| | | tgtttgagcttgtgggtgagccctccatatactgcaccagcaatgacgatcaagtgggcatctggagcggcccggcccctc |
| | | agtgcattatacctaacaaatgcacgcctccaaatgtggaaaatggaatattggtatctgacaacagaagcttattttcctt |
| | | aaatgaagttgtggagtttaggtgtcagcctggctttgtcatgaaaggaccccgccgtgtgaagtgccaggccctgaacaa |
| | | atgggagccggagctaccaagctgctccagggtatgtcagccacctccagatgtcctgcatgctgagcgtacccaaaggg |
| | | acaaggacaacttttcacccgggcaggaagtgttctacagctgtgagcccggctatgacctcagaggggctgcgtctatg |
| | | cgctgcacaccccagggagactggagccctgcagcccccacatgtgaagtgaaatcctgtgatgacttcatgggccaact |
| | | tcttaatggccgtgtgctatttccagtaaatctccagcttggagcaaaagtggattttgtttgtgatgaaggatttcaattaaa |
| | | aggcagctctgctagttattgtgtcttggctggaatggaaagcctttggaatagcagtgttccagtgtgtgaacaaatctttt |
| | | gtccaagtcctccagttattcctaatgggagacacacaggaaaacctctggaagtctttccctttggaaaagcagtaaatt |
| | | acacatgcgacccccacccagacagagggacgagcttcgacctcattggagagagcaccatccgctgcacaagtgaccc |
| | | tcaagggaatggggtttggagcagccctgcccctcgctgtggaattctgggtcactgtcaagccccagatcattttctgtttg |
| | | ccaagttgaaaacccaaaccaatgcatctgactttcccattgggacatctttaaagtacgaatgccgtcctgagtactacg |
| | | ggaggccattctctatcacatgtctagataacctggtctggtcaagtcccaaagatgtctgtaaacgtaaatcatgtaaaac |
| | | tcctccagatccagtgaatggcatggtgcatgtgatcacagacatccaggttggatccagaatcaactattcttgtactaca |
| | | gggcaccgactcattggtcactcatctgctgaatgtatcctctcaggcaatactgcccattggagcacgaagccgccaattt |
| | | gtcaacgaattccttgtgggctacccccaaccatcgccaatggagatttcattagcaccaacagagagaattttcactatg |
| | | gatcagtggtgacctaccgctgcaatcttggaagcagagggagaaaggtgtttgagcttgtgggtgagccctccatatact |
| | | gcaccagcaatgacgatcaagtgggcatctggagcggccccgcccctcagtgcattatatagcaattgaataaaagatcttt |
| | | attttcattagatctgtgtgttggttttttgtgtg |
| 62 | human | MGAAGLLGVFLALVAPGVLGISCGSPPPILNGRISYYSTPIAVGTVIRYSCSGTFRLI |
| | CR2 | GEKSLLCITKDKVDGTWDKPAPKCEYFNKYSSCPEPIVPGGYKIRGSTPYRHGDSV |
| | isoform | TFACKTNFSMNGNKSVWCQANNMWGPTRLPTCVSVFPLECPALPMIHNGHHTSE |
| | 1 | NVGSIAPGLSVTYSCESGYLLVGEKIINCLSSGKWSAVPPTCEEARCKSLGRFPNG |
| | | KVKEPPILRVGVTANFFCDEGYRLQGPPSSRCVIAGQGVAWTKMPVCEEIFCPSPP |
| | | PILNGRHIGNSLANVSYGSIVTYTCDPDPEEGVNFILIGESTLRCTVDSQKTGTWSG |
| | | PAPRCELSTSAVQCPHPQILRGRMVSGQKDRYTYNDTVIFACMFGFTLKGSKQIR |
| | | CNAQGTWEPSAPVCEKECQAPPNILNGQKEDRHMVRFDPGTSIKYSCNPGYVLV |
| | | GEESIQCTSEGVWTPPVPQCKVAACEATGRQLLTKPQHQFVRPDVNSSCGEGYKL |
| | | SGSVYQECQGTIPWFMEIRLCKEITCPPPPVIYNGAHTGSSLEDFPYGTTVTYTCNP |
| | | GPERGVEFSLIGESTIRCTSNDQERGTWSGPAPLCKLSLLAVQCSHVHIANGYKISG |
| | | KEAPYFYNDTVTFKCYSGFTLKGSSQIRCKADNTWDPEIPVCEKGCQSPPGLHHG |
| | | RHTGGNTVFFVSGMTVDYTCDPGYLLVGNKSIHCMPSGNWSPSAPRCEETCQHV |
| | | RQSLQELPAGSRVELVNTSCQDGYQLTGHAYQMCQDAENGIWFKKIPLCKVIHC |
| | | HPPPVIVNGKHTGMMAENFLYGNEVSYECDQGFYLLGEKKLQCRSDSKGHGSWS |
| | | GPSPQCLRSPPVTRCPNPEVKHGYKLNKTHSAYSHNDIVYVDCNPGFIMNGSRVIR |
| | | CHTDNTWVPGVPTCIKKAFIGCPPPPKTPNGNHTGGNIARFSPGMSILYSCDQGYL |
| | | LVGEALLLCTHEGTWSQPAPHCKEVNCSSPADMDGIQKGLEPRKMYQYGAVVTL |
| | | ECEDGYMLEGSPQSQCQSDHQWNPPLAVCRSRSLAPVLCGIAAGLILLTFLIVITL |
| | | YVISKHRARNYYTDTSQKEAFHLEAREVYSVDPYNPAS |
| 63 | human | MGAAGLLGVFLALVAPGVLGISCGSPPPILNGRISYYSTPIAVGTVIRYSCSGTFRLI |
| | CR2 | GEKSLLCITKDKVDGTWDKPAPKCEYFNKYSSCPEPIVPGGYKIRGSTPYRHGDSV |
| | isoform | TFACKTNFSMNGNKSVWCQANNMWGPTRLPTCVSVFPLECPALPMIHNGHHTSE |
| | 2 | NVGSIAPGLSVTYSCESGYLLVGEKIINCLSSGKWSAVPPTCEEARCKSLGRFPNG |
| | | KVKEPPILRVGVTANFFCDEGYRLQGPPSSRCVIAGQGVAWTKMPVCEEIFCPSPP |
| | | PILNGRHIGNSLANVSYGSIVTYTCDPDPEEGVNFILIGESTLRCTVDSQKTGTWSG |
| | | PAPRCELSTSAVQCPHPQILRGRMVSGQKDRYTYNDTVIFACMFGFTLKGSKQIR |
| | | CNAQGTWEPSAPVCEKECQAPPNILNGQKEDRHMVRFDPGTSIKYSCNPGYVLV |
| | | GEESIQCTSEGVWTPPVPQCKVAACEATGRQLLTKPQHQFVRPDVNSSCGEGYKL |
| | | SGSVYQECQGTIPWFMEIRLCKEITCPPPPVIYNGAHTGSSLEDFPYGTTVTYTCNP |
| | | GPERGVEFSLIGESTIRCTSNDQERGTWSGPAPLCKLSLLAVQCSHVHIANGYKISG |
| | | KEAPYFYNDTVTFKCYSGFTLKGSSQIRCKADNTWDPEIPVCEKETCQHVRQSLQ |
| | | ELPAGSRVELVNTSCQDGYQLTGHAYQMCQDAENGIWFKKIPLCKVIHCHPPPVI |
| | | VNGKHTGMMAENFLYGNEVSYECDQGFYLLGEKKLQCRSDSKGHGSWSGPSPQ |
| | | CLRSPPVTRCPNPEVKHGYKLNKTHSAYSHNDIVYVDCNPGFIMNGSRVIRCHTD |
| | | NTWVPGVPTCIKKAFIGCPPPPKTPNGNHTGGNIARFSPGMSILYSCDQGYLLVGE |
| | | ALLLCTHEGTWSQPAPHCKEVNCSSPADMDGIQKGLEPRKMYQYGAVVTLECED |
| | | GYMLEGSPQSQCQSDHQWNPPLAVCRSRSLAPVLCGIAAGLILLTFLIVITLYVISK |
| | | HRARNYYTDTSQKEAFHLEAREVYSVDPYNPAS |
| 64 | human | MGASSPRSPEPVGPPAPGLPFCCGGSLLAVVVLLALPVAWGQCNAPEWLPFARPT |
| | CR1 | NLTDEFEFPIGTYLNYECRPGYSGRPFSIICLKNSVWTGAKDRCRRKSCRNPPDPV |
| | isoform | NGMVHVIKGIQFGSQIKYSCTKGYRLIGSSSATCIISGDTVIWDNETPICDRIPCGLP |
| | 1 | PTITNGDFISTNRENFHYGSVVTYRCNPGSGGRKVFELVGEPSIYCTSNDDQVGIW |
| | | SGPAPQCIIPNKCTPPNVENGILVSDNRSLFSLNEVVEFRCQPGFVMKGPRRVKCQ |
| | | ALNKWEPELPSCSRVCQPPPDVLHAERTQRDKDNFSPGQEVFYSCEPGYDLRGAA |
| | | SMRCTPQGDWSPAAPTCEVKSCDDFMGQLLNGRVLFPVNLQLGAKVDFVCDEG |
| | | FQLKGSSASYCVLAGMESLWNSSVPVCEQIFCPSPPVIPNGRHTGKPLEVFPFGKT |
| | | VNYTCDPHPDRGTSFDLIGESTIRCTSDPQGNGVWSSPAPRCGILGHCQAPDHFLF |
| | | AKLKTQTNASDFPIGTSLKYECRPEYYGRPFSITCLDNLVWSSPKDVCKRKSCKTP |
| | | PDPVNGMVHVITDIQVGSRINYSCTTGHRLIGHSSAECILSGNAAHWSTKPPICQRI |
| | | PCGLPPTIANGDFISTNRENFHYGSVVTYRCNPGSGGRKVFELVGEPSIYCTSNDD |
| | | QVGIWSGPAPQCIIPNKCTPPNVENGILVSDNRSLFSLNEVVEFRCQPGFVMKGPR |
| | | RVKCQALNKWEPELPSCSRVCQPPPDVLHAERTQRDKDNFSPGQEVFYSCEPGYD |
| | | LRGAASMRCTPQGDWSPAAPTCEVKSCDDFMGQLLNGRVLFPVNLQLGAKVDF |
| | | VCDEGFQLKGSSASYCVLAGMESLWNSSVPVCEQIFCPSPPVIPNGRHTGKPLEVF |
| | | PFGKAVNYTCDPHPDRGTSFDLIGESTIRCTSDPQGNGVWSSPAPRCGILGHCQAP |
| | | DHFLFAKLKTQTNASDFPIGTSLKYECRPEYYGRPFSITCLDNLVWSSPKDVCKRK |
| | | SCKTPPDPVNGMVHVITDIQVGSRINYSCTTGHRLIGHSSAECILSGNTAHWSTKPP |
| | | ICQRIPCGLPPTIANGDFISTNRENFHYGSVVTYRCNLGSRGRKVFELVGEPSIYCTS |
| | | NDDQVGIWSGPAPQCIIPNKCTPPNVENGILVSDNRSLFSLNEVVEFRCQPGFVMK |
| | | GPRRVKCQALNKWEPELPSCSRVCQPPPEILHGEHTPSHQDNFSPGQEVFYSCEPG |
| | | YDLRGAASLHCTPQGDWSPEAPRCAVKSCDDFLGQLPHGRVLFPLNLQLGAKVS |
| | | FVCDEGFRLKGSSVSHCVLVGMRSLWNNSVPVCEHIFCPNPPAILNGRHTGTPSG |
| | | DIPYGKEISYTCDPHPDRGMTFNLIGESTIRCTSDPHGNGVWSSPAPRCELSVRAG |
| | | HCKTPEQFPFASPTIPINDFEFPVGTSLNYECRPGYFGKMFSISCLENLVWSSVEDN |
| | | CRRKSCGPPPEPFNGMVHINTDTQFGSTVNYSCNEGFRLIGSPSTTCLVSGNNVTW |
| | | DKKAPICEIISCEPPPTISNGDFYSNNRTSFHNGTVVTYQCHTGPDGEQLFELVGER |
| | | SIYCTSKDDQVGVWSSPPPRCISTNKCTAPEVENAIRVPGNRSFFTLTEIIRFRCQPG |
| | | FVMVGSHTVQCQTNGRWGPKLPHCSRVCQPPPEILHGEHTLSHQDNFSPGQEVFY |
| | | SCEPSYDLRGAASLHCTPQGDWSPEAPRCTVKSCDDFLGQLPHGRVLLPLNLQLG |
| | | AKVSFVCDEGFRLKGRSASHCVLAGMKALWNSSVPVCEQIFCPNPPAILNGRHTG |
| | | TPFGDIPYGKEISYACDTHPDRGMTFNLIGESSIRCTSDPQGNGVWSSPAPRCELSV |
| | | PAACPHPPKIQNGHYIGGHVSLYLPGMTISYICDPGYLLVGKGFIFCTDQGIWSQL |
| | | DHYCKEVNCSFPLFMNGISKELEMKKVYHYGDYVTLKCEDGYTLEGSPWSQCQ |
| | | ADDRWDPPLAKCTSRTHDALIVGTLSGTIFFILLIIFLSWIILKHRKGNNAHENPKE |
| | | VAIHLHSQGGSSVHPRTLQTNEENSRVLP |
| 65 | human | MGASSPRSPEPVGPPAPGLPFCCGGSLLAVVVLLALPVAWGQCNAPEWLPFARPT |
| | CR1 | NLTDEFEFPIGTYLNYECRPGYSGRPFSIICLKNSVWTGAKDRCRRKSCRNPPDPV |
| | isoform | NGMVHVIKGIQFGSQIKYSCTKGYRLIGSSSATCIISGDTVIWDNETPICDRIPCGLP |
| | 2 | PTITNGDFISTNRENFHYGSVVTYRCNPGSGGRKVFELVGEPSIYCTSNDDQVGIW |
| | | SGPAPQCIIPNKCTPPNVENGILVSDNRSLFSLNEVVEFRCQPGFVMKGPRRVKCQ |
| | | ALNKWEPELPSCSRVCQPPPDVLHAERTQRDKDNFSPGQEVFYSCEPGYDLRGAA |
| | | SMRCTPQGDWSPAAPTCEVKSCDDFMGQLLNGRVLFPVNLQLGAKVDFVCDEG |
| | | FQLKGSSASYCVLAGMESLWNSSVPVCEQIFCPSPPVIPNGRHTGKPLEVFPFGKT |
| | | VNYTCDPHPDRGTSFDLIGESTIRCTSDPQGNGVWSSPAPRCGILGHCQAPDHFLF |
| | | AKLKTQTNASDFPIGTSLKYECRPEYYGRPFSITCLDNLVWSSPKDVCKRKSCKTP |
| | | PDPVNGMVHVITDIQVGSRINYSCTTGHRLIGHSSAECILSGNAAHWSTKPPICQRI |
| | | PCGLPPTIANGDFISTNRENFHYGSVVTYRCNPGSGGRKVFELVGEPSIYCTSNDD |
| | | QVGIWSGPAPQCIIPNKCTPPNVENGILVSDNRSLFSLNEVVEFRCQPGFVMKGPR |
| | | RVKCQALNKWEPELPSCSRVCQPPPDVLHAERTQRDKDNFSPGQEVFYSCEPGYD |
| | | LRGAASMRCTPQGDWSPAAPTCEVKSCDDFMGQLLNGRVLFPVNLQLGAKVDF |
| | | VCDEGFQLKGSSASYCVLAGMESLWNSSVPVCEQIFCPSPPVIPNGRHTGKPLEVF |
| | | PFGKTVNYTCDPHPDRGTSFDLIGESTIRCTSDPQGNGVWSSPAPRCGILGHCQAP |
| | | DHFLFAKLKTQTNASDFPIGTSLKYECRPEYYGRPFSITCLDNLVWSSPKDVCKRK |
| | | SCKTPPDPVNGMVHVITDIQVGSRINYSCTTGHRLIGHSSAECILSGNAAHWSTKP |
| | | PICQRIPCGLPPTIANGDFISTNRENFHYGSVVTYRCNPGSGGRKVFELVGEPSIYCT |
| | | SNDDQVGIWSGPAPQCIIPNKCTPPNVENGILVSDNRSLFSLNEVVEFRCQPGFVM |
| | | KGPRRVKCQALNKWEPELPSCSRVCQPPPDVLHAERTQRDKDNFSPGQEVFYSCE |
| | | PGYDLRGAASMRCTPQGDWSPAAPTCEVKSCDDFMGQLLNGRVLFPVNLQLGA |
| | | KVDFVCDEGFQLKGSSASYCVLAGMESLWNSSVPVCEQIFCPSPPVIPNGRHTGKP |
| | | LEVFPFGKAVNYTCDPHPDRGTSFDLIGESTIRCTSDPQGNGVWSSPAPRCGILGH |
| | | CQAPDHFLFAKLKTQTNASDFPIGTSLKYECRPEYYGRPFSITCLDNLVWSSPKDV |
| | | CKRKSCKTPPDPVNGMVHVITDIQVGSRINYSCTTGHRLIGHSSAECILSGNTAHW |
| | | STKPPICQRIPCGLPPTIANGDFISTNRENFHYGSVVTYRCNLGSRGRKVFELVGEP |
| | | SIYCTSNDDQVGIWSGPAPQCIIPNKCTPPNVENGILVSDNRSLFSLNEVVEFRCQP |
| | | GFVMKGPRRVKCQALNKWEPELPSCSRVCQPPPEILHGEHTPSHQDNFSPGQEVF |
| | | YSCEPGYDLRGAASLHCTPQGDWSPEAPRCAVKSCDDFLGQLPHGRVLFPLNLQL |
| | | GAKVSFVCDEGFRLKGSSVSHCVLVGMRSLWNNSVPVCEHIFCPNPPAILNGRHT |
| | | GTPSGDIPYGKEISYTCDPHPDRGMTFNLIGESTIRCTSDPHGNGVWSSPAPRCELS |
| | | VRAGHCKTPEQFPFASPTIPINDFEFPVGTSLNYECRPGYFGKMFSISCLENLVWSS |
| | | VEDNCRRKSCGPPPEPFNGMVHINTDTQFGSTVNYSCNEGFRLIGSPSTTCLVSGN |
| | | NVTWDKKAPICEIISCEPPPTISNGDFYSNNRTSFHNGTVVTYQCHTGPDGEQLFEL |
| | | VGERSIYCTSKDDQVGVWSSPPPRCISTNKCTAPEVENAIRVPGNRSFFTLTEIIRFR |
| | | CQPGFVMVGSHTVQCQTNGRWGPKLPHCSRVCQPPPEILHGEHTLSHQDNFSPGQ |
| | | EVFYSCEPSYDLRGAASLHCTPQGDWSPEAPRCTVKSCDDFLGQLPHGRVLLPLN |
| | | LQLGAKVSFVCDEGFRLKGRSASHCVLAGMKALWNSSVPVCEQIFCPNPPAILNG |
| | | RHTGTPFGDIPYGKEISYACDTHPDRGMTFNLIGESSIRCTSDPQGNGVWSSPAPR |
| | | CELSVPAACPHPPKIQNGHYIGGHVSLYLPGMTISYICDPGYLLVGKGFIFCTDQGI |
| | | WSQLDHYCKEVNCSFPLFMNGISKELEMKKVYHYGDYVTLKCEDGYTLEGSPW |
| | | SQCQADDRWDPPLAKCTSRTHDALIVGTLSGTIFFILLIIFLSWIILKHRKGNNAHE |
| | | NPKEVAIHLHSQGGSSVHPRTLQTNEENSRVLP |
| 66 | human | MGASSPRSPEPVGPPAPGLPFCCGGSLLAVVVLLALPVAWGQCNAPEWLPFARPT |
| | CR1 | NLTDEFEFPIGTYLNYECRPGYSGRPFSIICLKNSVWTGAKDRCRRKSCRNPPDPV |
| | isoform | NGMVHVIKGIQFGSQIKYSCTKGYRLIGSSSATCIISGDTVIWDNETPICDRIPCGLP |
| | 3 | PTIANGDFISTNRENFHYGSVVTYRCNPGSGGRKVFELVGEPSIYCTSNDDQVGIW |
| | | SGPAPQCIIPNKCTPPNVENGILVSDNRSLFSLNEVVEFRCQPGFVMKGPRRVKCQ |
| | | ALNKWEPELPSCSRVCQPPPDVLHAERTQRDKDNFSPGQEVFYSCEPGYDLRGAA |
| | | SMRCTPQGDWSPAAPTCEVKSCDDFMGQLLNGRVLFPVNLQLGAKVDFVCDEG |
| | | FQLKGSSASYCVLAGMESLWNSSVPVCEQIFCPSPPVIPNGRHTGKPLEVFPFGKT |
| | | VNYTCDPHPDRGTSFDLIGESTIRCTSDPQGNGVWSSPAPRCGILGHCQAPDHFLF |
| | | AKLKTQTNASDFPIGTSLKYECRPEYYGRPFSITCLDNLVWSSPKDVCKRKSCKTP |
| | | PDPVNGMVHVITDIQVGSRINYSCTTGHRLIGHSSAECILSGNAAHWSTKPPICQRI |
| | | PCGLPPTIANGDFISTNRENFHYGSVVTYRCNPGSGGRKVFELVGEPSIYCTSNDD |
| | | QVGIWSGPAPQCIIPNKCTPPNVENGILVSDNRSLFSLNEVVEFRCQPGFVMKGPR |
| | | RVKCQALNKWEPELPSCSRVCQPPPDVLHAERTQRDKDNFSPGQEVFYSCEPGYD |
| | | LRGAASMRCTPQGDWSPAAPTCEVKSCDDFMGQLLNGRVLFPVNLQLGAKVDF |
| | | VCDEGFQLKGSSASYCVLAGMESLWNSSVPVCEQIFCPSPPVIPNGRHTGKPLEVF |
| | | PFGKAVNYTCDPHPDRGTSFDLIGESTIRCTSDPQGNGVWSSPAPRCGILGHCQAP |
| | | DHFLFAKLKTQTNASDFPIGTSLKYECRPEYYGRPFSITCLDNLVWSSPKDVCKRK |
| | | SCKTPPDPVNGMVHVITDIQVGSRINYSCTTGHRLIGHSSAECILSGNTAHWSTKPP |
| | | ICQRIPCGLPPTIANGDFISTNRENFHYGSVVTYRCNLGSRGRKVFELVGEPSIYCTS |
| | | NDDQVGIWSGPAPQCIIPNKCTPPNVENGILVSDNRSLFSLNEVVEFRCQPGFVMK |
| | | GPRRVKCQALNKWEPELPSCSRVCQPPPEILHGEHTPSHQDNFSPGQEVFYSCEPG |
| | | YDLRGAASLHCTPQGDWSPEAPRCAVKSCDDFLGQLPHGRVLFPLNLQLGAKVS |
| | | FVCDEGFRLKGSSVSHCVLVGMRSLWNNSVPVCEHIFCPNPPAILNGRHTGTPSG |
| | | DIPYGKEISYTCDPHPDRGMTFNLIGESTIRCTSDPHGNGVWSSPAPRCELSVRAG |
| | | HCKTPEQFPFASPTIPINDFEFPVGTSLNYECRPGYFGKMFSISCLENLVWSSVEDN |
| | | CRRKSCGPPPEPFNGMVHINTDTQFGSTVNYSCNEGFRLIGSPSTTCLVSGNNVTW |
| | | DKKAPICEIISCEPPPTISNGDFYSNNRTSFHNGTVVTYQCHTGPDGEQLFELVGER |
| | | SIYCTSKDDQVGVWSSPPPRCISTNKCTAPEVENAIRVPGNRSFFTLTEIIRFRCQPG |
| | | FVMVGSHTVQCQTNGRWGPKLPHCSRVCQPPPEILHGEHTLSHQDNFSPGQEVFY |
| | | SCEPSYDLRGAASLHCTPQGDWSPEAPRCTVKSCDDFLGQLPHGRVLLPLNLQLG |
| | | AKVSFVCDEGFRLKGRSASHCVLAGMKALWNSSVPVCEQIFCPNPPAILNGRHTG |
| | | TPFGDIPYGKEISYACDTHPDRGMTFNLIGESSIRCTSDPQGNGVWSSPAPRCELSV |
| | | PAACPHPPKIQNGHYIGGHVSLYLPGMTISYICDPGYLLVGKGFIFCTDQGIWSQL |
| | | DHYCKEVNCSFPLFMNGISKELEMKKVYHYGDYVTLKCEDGYTLEGSPWSQCQ |
| | | ADDRWDPPLAKCTSRTHDALIVGTLSGTIFFILLIIFLSWIILKHRKGNNAHENPKE |
| | | VAIHLHSQGGSSVHPRTLQTNEENSRVLP |
| 67 | human | MRLLAFLSLLALVLQETGT |
| | opticin | |
| | signal | |
| | ing- | |
| | peptide | |
| 68 | cyto- | GACATTGATTATTGACTAGTTATTAATAGTAATCAATTACGGGGTCATTAGTTC |
| | megalo- | ATAGCCCATATATGGAGTTCCGCGTTACATAACTTACGGTAAATGGCCCGCCT |
| | virus | GGCTGACCGCCCAACGACCCCCGCCCATTGACGTCAATAATGACGTATGTTCC |
| | (CMV) | CATAGTAACGCCAATAGGGACTTTCCATTGACGTCAATGGGTGGACTATTTAC |
| | immed- | GGTAAACTGCCCACTTGGCAGTACATCAAGTGTATCATATGCCAAGTACGCCC |
| | iate- | CCTATTGACGTCAATGACGGTAAATGGCCCGCCTGGCATTATGCCCAGTACAT |
| | early | GACCTTATGGGACTTTCCTACTTGGCAGTACATCTACGTATTAGTCATCGCTAT |
| | en- | TACCATGGTGATGCGGTTTTGGCAGTACATCAATGGGCGTGGATAGCGGTTTG |
| | hancer | ACTCACGGGGATTTCCAAGTCTCCACCCCATTGACGTCAATGGGAGTTTGTTTT |
| | and/or | GGCACCAAAATCAACGGGACTTTCCAAAATGTCGTAACAACTCCGCCCCATTG |
| | promo- | ACGCAAATGGGCGGTAGGCGTGTACGGTGGGAGGTCTATATAAGCAGAGCTC |
| | ter | GTTTAGTGAACCG |
| 69 | CMV en- | GACATTGATTATTGACTAGTTATTAATAGTAATCAATTACGGGGTCATTAGTTC |
| | hancer | ATAGCCCATATATGGAGTTCCGCGTTACATAACTTACGGTAAATGGCCCGCCT |
| | | GGCTGACCGCCCAACGACCCCCGCCCATTGACGTCAATAATGACGTATGTTCC |
| | | CATAGTAACGCCAATAGGGACTTTCCATTGACGTCAATGGGTGGACTATTTAC |
| | | GGTAAACTGCCCACTTGGCAGTACATCAAGTGTATCATATGCCAAGTACGCCC |
| | | CCTATTGACGTCAATGACGGTAAATGGCCCGCCTGGCATTATGCCCAGTACAT |
| | | GACCTTATGGGACTTTCCTACTTGGCAGTACATCTACGTATTAGTCATCGCTAT |
| | | TACCATG |
| 70 | Inter- | GCCCCTCTCCCTCCCCCCCCCCTAACGTTACTGGCCGAAGCCGCTTGGAATAA |
| | nal | GGCCGGTGTGCGTTTGTCTATATGTTATTTTCCACCATATTGCCGTCTTTTGGC |
| | Ribo- | AATGTGAGGGCCCGGAAACCTGGCCCTGTCTTCTTGACGAGCATTCCTAGGGG |
| | some | TCTTTCCCCTCTCGCCAAAGGAATGCAAGGTCTGTTGAATGTCGTGAAGGAAG |
| | Entry | CAGTTCCTCTGGAAGCTTCTTGAAGACAAACAACGTCTGTAGCGACCCTTTGC |
| | Site | AGGCAGCGGAACCCCCCACCTGGCGACAGGTGCCTCTGCGGCCAAAAGCCAC |
| | | GTGTATAAGATACACCTGCAAAGGCGGCACAACCCCAGTGCCACGTTGTGAG |
| | | TTGGATAGTTGTGGAAAGAGTCAAATGGCTCTCCTCAAGCGTATTCAACAAGG |
| | | GGCTGAAGGATGCCCAGAAGGTACCCCATTGTATGGGATCTGATCTGGGGCCT |
| | | CGGTACACATGCTTTACATGTGTTTAGTCGAGGTTAAAAAAACGTCTAGGCCC |
| | | CCCGAACCACGGGGACGTGGTTTTCCTTTGAAAAACACGATGATAATATGGCC |
| | | ACA |
| 71 | synthe- | AATAAAAGATCTTTATTTTCATTAGATCTGTGTGTTGGTTTTTTGTGTG |
| | tic | |
| | poly(A) | |
| | se- | |
| | quence | |
| 72 | bovine | CTGTGCCTTCTAGTTGCCAGCCATCTGTTGTTTGCCCCTCCCCCGTGCCTTCCTT |
| | growth | GACCCTGGAAGGTGCCACTCCCACTGTCCTTTCCTAATAAAATGAGGAAATTG |
| | hormone | CATCGCATTGTCTGAGTAGGTGTCATTCTATTCTGGGGGGTGGGGTGGGGCAG |
| | poly(A) | GACAGCAAGGGGGAGGATTGGGAAGACAATAGCAGGCATGCTGGGGATGCG |
| | se- | GTGGGCTCTATG |
| | quence | |
| 73 | human | gatctacgggtggcatccctgtgacccctccccagtgcctctcctggccctggaagttgccactccagtgcccaccagccttgtcc |
| | growth | taataaaattaagttgcatcattttgtctgactaggtgtccttctataatattatggggtggaggggggtggtatggagcaaggggca |
| | hormone | agttgggaagacaacctgtagggcctgcggggtctattgggaaccaagctggagtgcagtggcacaatcttggctcactgcaatc |
| | poly(A) | tccgcctcctgggttcaagcgattctcctgcctcagcctcccgagttgttgggattccaggcatgcatgaccaggctcagctaattttt |
| | se- | gtttttttggtagagacggggtttcaccatattggccaggctggtctccaactcctaatctcaggtgatctacccaccttggcctccca |
| | quence | aattgctgggattacaggcgtgaaccactgctcccttccctgtccttctg |
| 74 | CR2-CR1 | GACAGGGAGTTGCTTGGAC |
| | J | |
| | Forward | |
| 75 | CR2-CR1 | GGGAAACTCAAATTCATCAGTTAGG |
| | J | |
| | Reverse | |
| 76 | CR2-CR1 | CAGAATGGCTTCCATTTGCCAGGC |
| | J | |
| | Probe | |
| 77 | CR2CR1 | atttcttgtggctctcctccgcctatcctaaatggccggattagttattattctacccccattgctgttggtaccgtgataaggtacagtt |
| | #7 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttattccggaagacc |
| | | gttttctatcatctgcctaaaaaactcagtctggactggtgctaaggacaggtgcagacgtaaatcatgtcgtaatcctccagatcct |
| | | gtgaatggcatggtgcatgtgatcaaaggcatccagttcggatcccaaattaaatattcttgtactaaaggataccgactcattggttc |
| | | ctcgtctgccacatgcatcatctcaggtgatactgtcatttgggataatgaaacacctatttgtgacagaattccttgtgggctacccc |
| | | ccaccatcaccaatggagatttcattagcaccaacagagagaattttcactatggatcagtggtgacctaccgctgcaatcctggaa |
| | | gcggagggagaaaggtgtttgagcttgtgggtgagccctccatatactgcaccagcaatgacgatcaagtgggcatctggagcg |
| | | gccccgcccctcagtgcattatacctaacaaatgcacgcctccaaatgtggaaaatggaatattggtatctgacaacagaagcttat |
| | | tttccttaaatgaagttgtggagtttaggtgtcagcctggctttgtcatgaaaggaccccgccgtgtgaagtgccaggccctgaaca |
| | | aatgggagccggagctaccaagctgctccaggatcttttgtccaagtcctccagttattcctaatgggagacacacaggaaaacct |
| | | ctggaagtctttccctttgggaaaacagtaaattacacatgcgacccccacccagacagagggacgagcttcgacctcattggag |
| | | agagcaccatccgctgcacaagtgaccctcaagggaatggggtttggagcagccctgcccctcgctgtggaattctgggtcactg |
| | | tcaagccccagatcattttctgtttgccaagttgaaaacccaaaccaatgcatctgactttcccattgggacatctttaaagtacgaat |
| | | gccgtcctgagtactacgggaggccattctctatcacatgtctagataacctggtctggtcaagtcccaaagatgtctgtaaacgtaa |
| | | atcatgtaaaactcctccagatccagtgaatggcatggtgcatgtgatcacagacatccaggttggatccagaatcaactattcttgt |
| | | actacagggcaccgactcattggtcactcatctgctgaatgtatcctctcgggcaatgctgcccattggagcacgaagccgccaat |
| | | ttgtcaacgaattccttgtgggctaccccccaccatcgccaatggagatttcattagcaccaacagagagaattttcactatggatca |
| | | gtggtgacctaccgctgcaatcctggaagcggagggagaaaggtgtttgagcttgtgggtgagccctccatatactgcaccagca |
| | | atgacgatcaagtgggcatctggagcggcccggcccctcagtgcattatacctaacaaatgcacgcctccaaatgtggaaaatgg |
| | | aatattggtatctgacaacagaagcttattttccttaaatgaagttgtggagtttaggtgtcagcctggctttgtcatgaaaggaccccg |
| | | ccgtgtgaagtgccaggccctgaacaaatgggagccggagctaccaagctgctccaggatcttttgtccaagtcctccagttattc |
| | | ctaatgggagacacacaggaaaacctctggaagtctttccctttggaaaagcagtaaattacacatgcgacccccacccagacag |
| | | agggacgagcttcgacctcattggagagagcaccatccgctgcacaagtgaccctcaagggaatggggtttggagcagccctg |
| | | cccctcgctgtggaattctgggtcactgtcaagccccagatcattttctgtttgccaagttgaaaacccaaaccaatgcatctgacttt |
| | | cccattgggacatctttaaagtacgaatgccgtcctgagtactacgggaggccattctctatcacatgtctagataacctggtctggt |
| | | caagtcccaaagatgtctgtaaacgtaaatcatgtaaaactcctccagatccagtgaatggcatggtgcatgtgatcacagacatcc |
| | | aggttggatccagaatcaactattcttgtactacagggcaccgactcattggtcactcatctgctgaatgtatcctctcaggcaatact |
| | | gcccattggagcacgaagccgccaatttgtcaacgaattccttgtgggctacccccaaccatcgccaatggagatttcattagcac |
| | | caacagagagaattttcactatggatcagtggtgacctaccgctgcaatcttggaagcagagggagaaaggtgtttgagcttgtgg |
| | | gtgagccctccatatactgcaccagcaatgacgatcaagtgggcatctggagcggccccgcccctcagtgcattatatag |
| 78 | CR2CR1 | atttcttgtggctctcctccgcctatcctaaatggcaggattagttattattctacccccattgctgttggtacagtgataaggtacagtt |
| | #7 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctcttgagtgtccagcacttcctatgatccacaatggacatcacacaagtgag |
| | | aatgttggctccattgctccaggattgtctgtgacttacagctgtgaatctggttacttgcttgttggagaaaagatcattaactgtttgt |
| | | ctagtggaaaatggagtgctgtcccccccacatgtgaagaggcaaggtgtaaatctctaggaaggtttcccaatgggaaggtaaa |
| | | ggagcctccaattctcagggttggtgtaactgcaaactttttctgtgatgaagggtataggctgcaaggcccaccttctagtaggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttatagcggaagac |
| | | cattttctatcatctgcctaaaaaactcagtctggactggtgctaaggacaggtgcagaaggaaatcatgtaggaatcctccagatc |
| | | ctgtgaatggcatggtgcatgtgatcaaaggcatccagtttggatcccaaattaaatattcttgtactaaaggataccgcctcattggt |
| | | tcctcatctgccacatgcatcatctcaggtgatactgtcatttgggataatgaaacacctatttgtgacagaattccttgtgggctaccc |
| | | cccaccatcaccaatggagatttcattagcaccaacagagagaattttcactatggatcagtggtgacctaccgctgcaatcctgga |
| | | agcggagggagaaaggtgtttgagcttgtgggtgagccctccatatactgcaccagcaatgatgatcaagtgggcatctggagc |
| | | ggccccgcccctcagtgcattatacctaacaaatgcactcctccaaatgtggaaaatggaatattggtatctgacaacagaagctta |
| | | ttttccttaaatgaagttgtggagtttaggtgtcagcctggctttgtcatgaaaggaccccgcagggtgaagtgccaggccctgaac |
| | | aaatgggagccagagctaccaagctgctccaggatcttttgtccaagtcctccagttattcctaatgggagacacacaggaaaacc |
| | | tctggaagtctttccctttgggaaaacagtaaattacacatgtgacccccacccagacagagggactagctttgacctcattggaga |
| | | gagcaccatccgctgcacaagtgaccctcaagggaatggggtttggagcagccctgcccctagatgtggaattctgggtcactgt |
| | | caagccccagatcattttctgtttgccaagttgaaaacccaaaccaatgcatctgactttcccattgggacatctttaaagtatgaatg |
| | | ccgccctgagtactatgggaggccattctctatcacatgtctagataacctggtctggtcaagtcccaaagatgtctgtaaaaggaa |
| | | atcatgtaaaactcctccagatccagtgaatggcatggtgcatgtgatcacagacatccaggttggatccagaatcaactattcttgt |
| | | actactgggcaccgcctcattggtcactcatctgctgaatgtatcctcagtggcaatgctgcccattggagcactaagccgccaattt |
| | | gtcaaagaattccttgtgggctaccccccaccattgccaatggagatttcattagcaccaacagagagaattttcactatggatcagt |
| | | ggtgacctaccgctgcaatcctggaagcggagggagaaaggtgtttgagcttgtgggtgagccctccatatactgcaccagcaat |
| | | gatgatcaagtgggcatctggagcggcccagcccctcagtgcattatacctaacaaatgcactcctccaaatgtggaaaatggaat |
| | | attggtatctgacaacagaagcttattttccttaaatgaagttgtggagtttaggtgtcagcctggctttgtcatgaaaggaccccgca |
| | | gggtgaagtgccaggccctgaacaaatgggagccagagctaccaagctgctccaggatcttttgtccaagtcctccagttattcct |
| | | aatgggagacacacaggaaaacctctggaagtctttccctttggaaaagcagtaaattacacatgtgacccccacccagacagag |
| | | ggactagctttgacctcattggagagagcaccatccgctgcacaagtgaccctcaagggaatggggtttggagcagccctgccc |
| | | ctagatgtggaattctgggtcactgtcaagccccagatcattttctgtttgccaagttgaaaacccaaaccaatgcatctgactttccc |
| | | attgggacatctttaaagtatgaatgccgccctgagtactatgggaggccattctctatcacatgtctagataacctggtctggtcaag |
| | | tcccaaagatgtctgtaaaaggaaatcatgtaaaactcctccagatccagtgaatggcatggtgcatgtgatcacagacatccaggt |
| | | tggatccagaatcaactattcttgtactactgggcaccgcctcattggtcactcatctgctgaatgtatcctctcaggcaatactgccc |
| | | attggagcactaagccgccaatttgtcaaagaattccttgtgggctacccccaaccattgccaatggagatttcattagcaccaaca |
| | | gagagaattttcactatggatcagtggtgacctaccgctgcaatcttggaagcagagggagaaaggtgtttgagcttgtgggtgag |
| | | ccctccatatactgcaccagcaatgatgatcaagtgggcatctggagcggccccgcccctcagtgcattatatag |
| 79 | CR2CR1 | atttcttgtggctctcctccgcctatcctaaatggccggattagttattattctacccccattgctgttggtaccgtgataaggtacagtt |
| | #8 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttggtggaggtggatcgggaggtggtggatc |
| | | tcaatgcaatgccccagaatggcttccatttgccaggcctaccaacctaactgatgaatttgagtttcccattgggacatatctgaact |
| | | atgaatgccgccctggttattccggaagaccgttttctatcatctgcctaaaaaactcagtctggactggtgctaaggacaggtgca |
| | | gacgtaaatcatgtcgtaatcctccagatcctgtgaatggcatggtgcatgtgatcaaaggcatccagttcggatcccaaattaaata |
| | | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccaggatcttttgtccaagtcctccagtt |
| | | attcctaatgggagacacacaggaaaacctctggaagtctttccctttgggaaaacagtaaattacacatggacccccacccaga |
| | | cagagggacgagcttcgacctcattggagagagcaccatccgctgcacaagtgaccctcaagggaatggggtttggagcagcc |
| | | ctgcccctcgctgtggaattctgggtcactgtcaagccccagatcattttctgtttgccaagttgaaaacccaaaccaatgcatctga |
| | | ctttcccattgggacatctttaaagtacgaatgccgtcctgagtactacgggaggccattctctatcacatgtctagataacctggtct |
| | | ggtcaagtcccaaagatgtctgtaaacgtaaatcatgtaaaactcctccagatccagtgaatggcatggtgcatgtgatcacagaca |
| | | tccaggttggatccagaatcaactattcttgtactacagggcaccgactcattggtcactcatctgctgaatgtatcctctcgggcaat |
| | | gctgcccattggagcacgaagccgccaatttgtcaacgaattccttgtgggctaccccccaccatcgccaatggagatttcattagc |
| | | accaacagagagaattttcactatggatcagtggtgacctaccgctgcaatcctggaagcggagggagaaaggtgtttgagcttgt |
| | | gggtgagccctccatatactgcaccagcaatgacgatcaagtgggcatctggagcggcccggcccctcagtgcattatacctaac |
| | | aaatgcacgcctccaaatgtggaaaatggaatattggtatctgacaacagaagcttattttccttaaatgaagttgtggagtttaggtg |
| | | tcagcctggctttgtcatgaaaggaccccgccgtgtgaagtgccaggccctgaacaaatgggagccggagctaccaagctgctc |
| | | caggatcttttgtccaagtcctccagttattcctaatgggagacacacaggaaaacctctggaagtctttccctttggaaaagcagta |
| | | aattacacatgcgacccccacccagacagagggacgagcttcgacctcattggagagagcaccatccgctgcacaagtgaccct |
| | | caagggaatggggtttggagcagccctgcccctcgctgtggaattctgggtcactgtcaagccccagatcattttctgtttgccaag |
| | | ttgaaaacccaaaccaatgcatctgactttcccattgggacatctttaaagtacgaatgccgtcctgagtactacgggaggccattct |
| | | ctatcacatgtctagataacctggtctggtcaagtcccaaagatgtctgtaaacgtaaatcatgtaaaactcctccagatccagtgaat |
| | | ggcatggtgcatgtgatcacagacatccaggttggatccagaatcaactattcttgtactacagggcaccgactcattggtcactcat |
| | | ctgctgaatgtatcctctcaggcaatactgcccattggagcacgaagccgccaatttgtcaacgaattccttgtgggctacccccaa |
| | | ccatcgccaatggagatttcattagcaccaacagagagaattttcactatggatcagtggtgacctaccgctgcaatcttggaagca |
| | | gagggagaaaggtgtttgagcttgtgggtgagccctccatatactgcaccagcaatgacgatcaagtgggcatctggagcggcc |
| | | ccgcccctcagtgcattatatag |
| 80 | CR2CR1 | atttcttgtggctctcctccgcctatcctaaatggcaggattagttattattctacccccattgctgttggtacagtgataaggtacagtt |
| | #8 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctcttgagtgtccagcacttcctatgatccacaatggacatcacacaagtgag |
| | | aatgttggctccattgctccaggattgtctgtgacttacagctgtgaatctggttacttgcttgttggagaaaagatcattaactgtttgt |
| | | ctagtggaaaatggagtgctgtcccccccacatgtgaagaggcaaggtgtaaatctctaggaaggtttcccaatgggaaggtaaa |
| | | ggagcctccaattctcagggttggtgtaactgcaaactttttctgtgatgaagggtataggctgcaaggcccaccttctagtaggtgt |
| | | gtaattgctggacagggagttgcttggaccaaaatgccagtatgtgaagaaatttttggtggaggtggatcaggaggtggtggatc |
| | | tcaatgcaatgccccagaatggcttccatttgccaggcctaccaacctaactgatgaatttgagtttcccattgggacatatctgaact |
| | | atgaatgccgccctggttatagcggaagaccattttctatcatctgcctaaaaaactcagtctggactggtgctaaggacaggtgca |
| | | gaaggaaatcatgtaggaatcctccagatcctgtgaatggcatggtgcatgtgatcaaaggcatccagtttggatcccaaattaaat |
| | | attcttgtactaaaggataccgcctcattggttccagttctgccacatgcatcatctcaggtgatactgtcatttgggataatgaaacac |
| | | ctatttgtgacagaattccttgtgggctaccccccaccatcaccaatggagatttcattagcaccaacagagagaattttcactatgg |
| | | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccaggatcttttgtccaagtcctccagt |
| | | tattcctaatgggagacacacaggaaaacctctggaagtctttccctttgggaaaacagtaaattacacatgtgacccccacccaga |
| | | cagagggactagctttgacctcattggagagagcaccatccgctgcacaagtgaccctcaagggaatggggtttggagcagccc |
| | | tgcccctagatgtggaattctgggtcactgtcaagccccagatcattttctgtttgccaagttgaaaacccaaaccaatgcatctgact |
| | | ttcccattgggacatctttaaagtatgaatgccgccctgagtactatgggaggccattctctatcacatgtctagataacctggtctgg |
| | | tcaagtcccaaagatgtctgtaaaaggaaatcatgtaaaactcctccagatccagtgaatggcatggtgcatgtgatcacagacatc |
| | | caggttggatccagaatcaactattcttgtactactgggcaccgcctcattggtcactcatctgctgaatgtatcctctcaggcaatgc |
| | | tgcccattggagcactaagccgccaatttgtcaaagaattccttgtgggctaccccccaccattgccaatggagatttcattagcacc |
| | | aacagagagaattttcactatggatcagtggtgacctaccgctgcaatcctggaagcggagggagaaaggtgtttgagcttgtgg |
| | | gtgagccctccatatactgcaccagcaatgatgatcaagtgggcatctggagcggcccagcccctcagtgcattatacctaacaa |
| | | atgcactcctccaaatgtggaaaatggaatattggtatctgacaacagaagcttattttccttaaatgaagttgtggagtttaggtgtca |
| | | gcctggctttgtcatgaaaggaccccgcagggtgaagtgccaggccctgaacaaatgggagccagagctaccaagctgctcca |
| | | ggatcttttgtccaagtcctccagttattcctaatgggagacacacaggaaaacctctggaagtctttccctttggaaaagcagtaaat |
| | | tacacatgtgacccccacccagacagagggactagctttgacctcattggagagagcaccatccgctgcacaagtgaccctcaa |
| | | gggaatggggtttggagcagccctgcccctagatgtggaattctgggtcactgtcaagccccagatcattttctgtttgccaagttga |
| | | aaacccaaaccaatgcatctgactttcccattgggacatctttaaagtatgaatgccgccctgagtactatgggaggccattctctat |
| | | cacatgtctagataacctggtctggtcaagtcccaaagatgtctgtaaaaggaaatcatgtaaaactcctccagatccagtgaatgg |
| | | catggtgcatgtgatcacagacatccaggttggatccagaatcaactattcttgtactactgggcaccgcctcattggtcactcatct |
| | | gctgaatgtatcctctcaggcaatactgcccattggagcactaagccgccaatttgtcaaagaattccttgtgggctacccccaacc |
| | | attgccaatggagatttcattagcaccaacagagagaattttcactatggatcagtggtgacctaccgctgcaatcttggaagcaga |
| | | gggagaaaggtgtttgagcttgtgggtgagccctccatatactgcaccagcaatgatgatcaagtgggcatctggagcggccccg |
| | | cccctcagtgcattatatag |
| 81 | CR2CR1 | atttcttgtggctctcctccgcctatcctaaatggcaggattagttattattctacccccattgctgttggtacagtgataaggtacagtt |
| | #8 (C) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctcttgagtgtccagcacttcctatgatccacaatggacatcacacaagtgag |
| | | aatgttggctccattgctccaggattgtctgtgacttacagctgtgaatctggttacttgcttgttggagaaaagatcattaactgtttgt |
| | | ctagtggaaaatggagtgctgtcccccccacatgtgaagaggcaaggtgtaaatctctaggaaggtttcccaatgggaaggtaaa |
| | | ggagcctccaattctcagggttggtgtaactgcaaactttttctgtgatgaagggtataggctgcaaggcccaccttctagtaggtgt |
| | | gtaattgctggacagggagttgcttggaccaaaatgccagtatgtgaagaaatttttggtggaggtggatcaggaggtggtggatc |
| | | tcaatgcaatgccccagaatggcttccatttgccaggcctaccaacctaactgatgaatttgagtttcccattgggacatatctgaact |
| | | atgaatgccgccctggttatagcggaagaccattttctatcatctgcctaaaaaactcagtctggactggtgctaaggacaggtgca |
| | | gaaggaaatcatgtaggaatcctccagatcctgtgaatggcatggtgcatgtgatcaaaggcatccagtttggatcccaaattaaat |
| | | attcttgtactaaaggataccgcctcattggttccagttctgccacatgcatcatctcaggtgatactgtcatttgggataatgaaacac |
| | | ctatttgtgacagaattccttgtgggctaccccccaccatcaccaatggagatttcattagcaccaacagagagaattttcactatgg |
| | | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccaggatcttttgtccaagtcctccagt |
| | | tattcctaatgggagacacacaggaaaacctctggaagtctttccctttgggaaaacagtaaattacacatgtgacccccacccaga |
| | | cagagggactagctttgacctcattggagagagcaccatccgctgcacaagtgaccctcaagggaatggggtttggagcagccc |
| | | tgcccctagatgtggaattctgggtcactgtcaagccccagatcattttctgtttgccaagttgaaaacccaaaccaatgcatctgact |
| | | ttcccattgggacatctttaaagtatgaatgccgccctgagtactatgggaggccattctctatcacatgtctagataacctggtctgg |
| | | tcaagtcccaaagatgtctgtaaaaggaaatcatgtaaaactcctccagatccagtgaatggcatggtgcatgtgatcacagacatc |
| | | caggttggatccagaatcaactattcttgtactactgggcaccgcctcattggtcactcatctgctgaatgtatcctctcaggcaatgc |
| | | tgcccattggagcactaagccgccaatttgtcaaagaattccttgtgggctaccccccaccattgccaatggagatttcattagcacc |
| | | aacagagagaattttcactatggatcagtggtgacctaccgctgcaatcctggaagcggagggagaaaggtgtttgagcttgtgg |
| | | gtgagccctccatatactgcaccagcaatgatgatcaagtgggcatctggagcggcccagcccctcagtgcattatacctaacaa |
| | | atgcactcctccaaatgtggaaaatggaatattggtatctgacaacagaagcttattttccttaaatgaagttgtggagtttaggtgtca |
| | | gcctggctttgtcatgaaaggaccccgcagggtgaagtgccaggccctgaacaaatgggagccagagctaccaagctgctcca |
| | | ggatcttttgtccaagtcctccagttattcctaatgggagacacacaggaaaacctctggaagtctttccctttggaaaagcagtaaat |
| | | tacacatgtgacccccacccagacagagggactagctttgacctcattggagagagcaccatccgctgcacaagtgaccctcaa |
| | | gggaatggggtttggagcagccctgcccctagatgtggaattctgggtcactgtcaagccccagatcattttctgtttgccaagttga |
| | | aaacccaaaccaatgcatctgactttcccattgggacatctttaaagtatgaatgccgccctgagtactatgggaggccattctctat |
| | | cacatgtctagataacctggtctggtcaagtcccaaagatgtctgtaaaaggaaatcatgtaaaactcctccagatccagtgaatgg |
| | | catggtgcatgtgatcacagacatccaggttggatccagaatcaactattcttgtactactgggcaccgcctcattggtcactcatct |
| | | gctgaatgtatcctctcaggcaatactgcccattggagcactaagccgccaatttgtcaaagaattccttgtgggctacccccaacc |
| | | attgccaatggagatttcattagcaccaacagagagaattttcactatggatcagtggtgacctaccgctgcaatcttggaagcaga |
| | | gggagaaaggtgtttgagcttgtgggtgagccctccatatactgcaccagcaatgatgatcaagtgggcatctggagcggccccg |
| | | cccctcagtgcattatatag |
| 82 | CR2CR1 | atttcttgtggctctcctccgcctatcctaaatggccggattagttattattctacccccattgctgttggtaccgtgataaggtacagtt |
| | #9 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttggtggaggtggatcgggaggtggtggatc |
| | | tcaatgcaatgccccagaatggcttccatttgccaggcctaccaacctaactgatgaatttgagtttcccattgggacatatctgaact |
| | | atgaatgccgccctggttattccggaagaccgttttctatcatctgcctaaaaaactcagtctggactggtgctaaggacaggtgca |
| | | gacgtaaatcatgtcgtaatcctccagatcctgtgaatggcatggtgcatgtgatcaaaggcatccagttcggatcccaaattaaata |
| | | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccaggggtggaggtggatcgggaggt |
| | | ggtggatctatcttttgtccaagtcctccagttattcctaatgggagacacacaggaaaacctctggaagtctttccctttgggaaaac |
| | | agtaaattacacatgcgacccccacccagacagagggacgagcttcgacctcattggagagagcaccatccgctgcacaagtg |
| | | accctcaagggaatggggtttggagcagccctgcccctcgctgtggaattctgggtcactgtcaagccccagatcattttctgtttg |
| | | ccaagttgaaaacccaaaccaatgcatctgactttcccattgggacatctttaaagtacgaatgccgtcctgagtactacgggaggc |
| | | cattctctatcacatgtctagataacctggtctggtcaagtcccaaagatgtctgtaaacgtaaatcatgtaaaactcctccagatcca |
| | | gtgaatggcatggtgcatgtgatcacagacatccaggttggatccagaatcaactattcttgtactacagggcaccgactcattggt |
| | | cactcatctgctgaatgtatcctctcgggcaatgctgcccattggagcacgaagccgccaatttgtcaacgaattccttgtgggctac |
| | | cccccaccatcgccaatggagatttcattagcaccaacagagagaattttcactatggatcagtggtgacctaccgctgcaatcctg |
| | | gaagcggagggagaaaggtgtttgagcttgtgggtgagccctccatatactgcaccagcaatgacgatcaagtgggcatctgga |
| | | gcggcccggcccctcagtgcattatacctaacaaatgcacgcctccaaatgtggaaaatggaatattggtatctgacaacagaag |
| | | cttattttccttaaatgaagttgtggagtttaggtgtcagcctggctttgtcatgaaaggaccccgccgtgtgaagtgccaggccctg |
| | | aacaaatgggagccggagctaccaagctgctccaggggtggaggtggatcgggaggtggtggatctatcttttgtccaagtcctc |
| | | cagttattcctaatgggagacacacaggaaaacctctggaagtctttccctttggaaaagcagtaaattacacatgcgacccccacc |
| | | cagacagagggacgagcttcgacctcattggagagagcaccatccgctgcacaagtgaccctcaagggaatggggtttggagc |
| | | agccctgcccctcgctgtggaattctgggtcactgtcaagccccagatcattttctgtttgccaagttgaaaacccaaaccaatgcat |
| | | ctgactttcccattgggacatctttaaagtacgaatgccgtcctgagtactacgggaggccattctctatcacatgtctagataacctg |
| | | gtctggtcaagtcccaaagatgtctgtaaacgtaaatcatgtaaaactcctccagatccagtgaatggcatggtgcatgtgatcaca |
| | | gacatccaggttggatccagaatcaactattcttgtactacagggcaccgactcattggtcactcatctgctgaatgtatcctctcagg |
| | | caatactgcccattggagcacgaagccgccaatttgtcaacgaattccttgtgggctacccccaaccatcgccaatggagatttcat |
| | | tagcaccaacagagagaattttcactatggatcagtggtgacctaccgctgcaatcttggaagcagagggagaaaggtgtttgag |
| | | cttgtgggtgagccctccatatactgcaccagcaatgacgatcaagtgggcatctggagcggccccgcccctcagtgcattatata |
| | | g |
| 83 | CR2CR1 | atttcttgtggctctcctccgcctatcctaaatggcaggattagttattattctacccccattgctgttggtacagtgataaggtacagtt |
| | #9 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctcttgagtgtccagcacttcctatgatccacaatggacatcacacaagtgag |
| | | aatgttggctccattgctccaggattgtctgtgacttacagctgtgaatctggttacttgcttgttggagaaaagatcattaactgtttgt |
| | | ctagtggaaaatggagtgctgtcccccccacatgtgaagaggcaaggtgtaaatctctaggaaggtttcccaatgggaaggtaaa |
| | | ggagcctccaattctcagggttggtgtaactgcaaactttttctgtgatgaagggtataggctgcaaggcccaccttctagtaggtgt |
| | | gtaattgctggacagggagttgcttggaccaaaatgccagtatgtgaagaaatttttggtggaggtggatcaggaggtggtggatc |
| | | tcaatgcaatgccccagaatggcttccatttgccaggcctaccaacctaactgatgaatttgagtttcccattgggacatatctgaact |
| | | atgaatgccgccctggttatagcggaagaccattttctatcatctgcctaaaaaactcagtctggactggtgctaaggacaggtgca |
| | | gaaggaaatcatgtaggaatcctccagatcctgtgaatggcatggtgcatgtgatcaaaggcatccagtttggatcccaaattaaat |
| | | attcttgtactaaaggataccgcctcattggttccagttctgccacatgcatcatctcaggtgatactgtcatttgggataatgaaacac |
| | | ctatttgtgacagaattccttgtgggctaccccccaccatcaccaatggagatttcattagcaccaacagagagaattttcactatgg |
| | | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccaggggtggaggtggatcaggag |
| | | gtggtggatctatcttttgtccaagtcctccagttattcctaatgggagacacacaggaaaacctctggaagtctttccctttgggaaa |
| | | acagtaaattacacatgtgacccccacccagacagagggactagctttgacctcattggagagagcaccatccgctgcacaagtg |
| | | accctcaagggaatggggtttggagcagccctgcccctagatgtggaattctgggtcactgtcaagccccagatcattttctgtttg |
| | | ccaagttgaaaacccaaaccaatgcatctgactttcccattgggacatctttaaagtatgaatgccgccctgagtactatgggaggc |
| | | cattctctatcacatgtctagataacctggtctggtcaagtcccaaagatgtctgtaaaaggaaatcatgtaaaactcctccagatcca |
| | | gtgaatggcatggtgcatgtgatcacagacatccaggttggatccagaatcaactattcttgtactactgggcaccgcctcattggtc |
| | | actcatctgctgaatgtatcctcagtggcaatgctgcccattggagcactaagccgccaatttgtcaaagaattccttgtgggctacc |
| | | ccccaccattgccaatggagatttcattagcaccaacagagagaattttcactatggatcagtggtgacctaccgctgcaatcctgg |
| | | aagcggagggagaaaggtgtttgagcttgtgggtgagccctccatatactgcaccagcaatgatgatcaagtgggcatctggagc |
| | | ggcccagcccctcagtgcattatacctaacaaatgcactcctccaaatgtggaaaatggaatattggtatctgacaacagaagctta |
| | | ttttccttaaatgaagttgtggagtttaggtgtcagcctggctttgtcatgaaaggaccccgcagggtgaagtgccaggccctgaac |
| | | aaatgggagccagagctaccaagctgctccaggggtggaggtggatcaggaggtggtggatctatcttttgtccaagtcctccag |
| | | ttattcctaatgggagacacacaggaaaacctctggaagtctttccctttggaaaagcagtaaattacacatgtgacccccacccag |
| | | acagagggactagctttgacctcattggagagagcaccatccgctgcacaagtgaccctcaagggaatggggtttggagcagcc |
| | | ctgcccctagatgtggaattctgggtcactgtcaagccccagatcattttctgtttgccaagttgaaaacccaaaccaatgcatctga |
| | | ctttcccattgggacatctttaaagtatgaatgccgccctgagtactatgggaggccattctctatcacatgtctagataacctggtctg |
| | | gtcaagtcccaaagatgtctgtaaaaggaaatcatgtaaaactcctccagatccagtgaatggcatggtgcatgtgatcacagacat |
| | | ccaggttggatccagaatcaactattcttgtactactgggcaccgcctcattggtcactcatctgctgaatgtatcctctcaggcaata |
| | | ctgcccattggagcactaagccgccaatttgtcaaagaattccttgtgggctacccccaaccattgccaatggagatttcattagcac |
| | | caacagagagaattttcactatggatcagtggtgacctaccgctgcaatcttggaagcagagggagaaaggtgtttgagcttgtgg |
| | | gtgagccctccatatactgcaccagcaatgatgatcaagtgggcatctggagcggccccgcccctcagtgcattatatag |
| 84 | CR2CR1 | atttcttgtggctctcctccgcctatcctaaatggccggattagttattattctacccccattgctgttggtaccgtgataaggtacagtt |
| | #10 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttattccggaagacc |
| | | gttttctatcatctgcctaaaaaactcagtctggactggtgctaaggacaggtgcagacgtaaatcatgtcgtaatcctccagatcct |
| | | gtgaatggcatggtgcatgtgatcaaaggcatccagttcggatcccaaattaaatattcttgtactaaaggataccgactcattggttc |
| | | ctcgtctgccacatgcatcatctcaggtgatactgtcatttgggataatgaaacacctatttgtgacagaattccttgtgggctacccc |
| | | ccaccatcaccaatggagatttcattagcaccaacagagagaattttcactatggatcagtggtgacctaccgctgcaatcctggaa |
| | | gcggagggagaaaggtgtttgagcttgtgggtgagccctccatatactgcaccagcaatgacgatcaagtgggcatctggagcg |
| | | gccccgcccctcagtgcattatacctggtggaggtggatcgggaggtggtggatctggtggaggtggatcgggaggtggtggat |
| | | ctggtggaggtggatcgggaggtggtggatctggtggaggtggatcgggaggtggtggatctggtggaggtggatcgggaggt |
| | | ggtggatctggtggaggtggatcgggaggtggtggatctatcttttgtccaagtcctccagttattcctaatgggagacacacagga |
| | | aaacctctggaagtctttccctttgggaaaacagtaaattacacatgcgacccccacccagacagagggacgagcttcgacctcat |
| | | tggagagagcaccatccgctgcacaagtgaccctcaagggaatggggtttggagcagccctgcccctcgctgtggaattctggg |
| | | tcactgtcaagccccagatcattttctgtttgccaagttgaaaacccaaaccaatgcatctgactttcccattgggacatctttaaagta |
| | | cgaatgccgtcctgagtactacgggaggccattctctatcacatgtctagataacctggtctggtcaagtcccaaagatgtctgtaaa |
| | | cgtaaatcatgtaaaactcctccagatccagtgaatggcatggtgcatgtgatcacagacatccaggttggatccagaatcaactat |
| | | tcttgtactacagggcaccgactcattggtcactcatctgctgaatgtatcctctcgggcaatgctgcccattggagcacgaagccg |
| | | ccaatttgtcaacgaattccttgtgggctaccccccaccatcgccaatggagatttcattagcaccaacagagagaattttcactatg |
| | | gatcagtggtgacctaccgctgcaatcctggaagcggagggagaaaggtgtttgagcttgtgggtgagccctccatatactgcac |
| | | cagcaatgacgatcaagtgggcatctggagcggcccggcccctcagtgcattatacctggtggaggtggatcgggaggtggtg |
| | | gatctggtggaggtggatcgggaggtggtggatctggtggaggtggatcgggaggtggtggatctggtggaggtggatcggga |
| | | ggtggtggatctggtggaggtggatcgggaggtggtggatctggtggaggtggatcgggaggtggtggatctatcttttgtccaa |
| | | gtcctccagttattcctaatgggagacacacaggaaaacctctggaagtctttccctttggaaaagcagtaaattacacatgcgacc |
| | | cccacccagacagagggacgagcttcgacctcattggagagagcaccatccgctgcacaagtgaccctcaagggaatggggtt |
| | | tggagcagccctgcccctcgctgtggaattctgggtcactgtcaagccccagatcattttctgtttgccaagttgaaaacccaaacc |
| | | aatgcatctgactttcccattgggacatctttaaagtacgaatgccgtcctgagtactacgggaggccattctctatcacatgtctaga |
| | | taacctggtctggtcaagtcccaaagatgtctgtaaacgtaaatcatgtaaaactcctccagatccagtgaatggcatggtgcatgtg |
| | | atcacagacatccaggttggatccagaatcaactattcttgtactacagggcaccgactcattggtcactcatctgctgaatgtatcct |
| | | ctcaggcaatactgcccattggagcacgaagccgccaatttgtcaacgaattccttgtgggctacccccaaccatcgccaatgga |
| | | gatttcattagcaccaacagagagaattttcactatggatcagtggtgacctaccgctgcaatcttggaagcagagggagaaaggt |
| | | gtttgagcttgtgggtgagccctccatatactgcaccagcaatgacgatcaagtgggcatctggagcggccccgcccctcagtgc |
| | | attatatag |
| 85 | CR2CR1 | atttcttgtggctctcctccgcctatcctaaatggccggattagttattattctacccccattgctgttggtaccgtgataaggtacagtt |
| | #10 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttattccggaagacc |
| | | gttttctatcatctgcctaaaaaactcagtctggactggtgctaaggacaggtgcagacgtaaatcatgtcgtaatcctccagatcct |
| | | gtgaatggcatggtgcatgtgatcaaaggcatccagttcggatcccaaattaaatattcttgtactaaaggataccgactcattggttc |
| | | ctcgtctgccacatgcatcatctcaggtgatactgtcatttgggataatgaaacacctatttgtgacagaattccttgtgggctacccc |
| | | ccaccatcaccaatggagatttcattagcaccaacagagagaattttcactatggatcagtggtgacctaccgctgcaatcctggaa |
| | | gcggagggagaaaggtgtttgagcttgtgggtgagccctccatatactgcaccagcaatgacgatcaagtgggcatctggagcg |
| | | gccccgcccctcagtgcattatacctggtggaggtggatcgggaggtggtggatctggtggaggtggatcgggaggtggtggat |
| | | ctggtggaggtggatcgatcttttgtccaagtcctccagttattcctaatgggagacacacaggaaaacctctggaagtctttcccttt |
| | | gggaaaacagtaaattacacatgcgacccccacccagacagagggacgagcttcgacctcattggagagagcaccatccgctg |
| | | cacaagtgaccctcaagggaatggggtttggagcagccctgcccctcgctgtggaattctgggtcactgtcaagccccagatcatt |
| | | ttctgtttgccaagttgaaaacccaaaccaatgcatctgactttcccattgggacatctttaaagtacgaatgccgtcctgagtactac |
| | | gggaggccattctctatcacatgtctagataacctggtctggtcaagtcccaaagatgtctgtaaacgtaaatcatgtaaaactcctc |
| | | cagatccagtgaatggcatggtgcatgtgatcacagacatccaggttggatccagaatcaactattcttgtactacagggcaccga |
| | | ctcattggtcactcatctgctgaatgtatcctctcgggcaatgctgcccattggagcacgaagccgccaatttgtcaacgaattcctt |
| | | gtgggctaccccccaccatcgccaatggagatttcattagcaccaacagagagaattttcactatggatcagtggtgacctaccgct |
| | | gcaatcctggaagcggagggagaaaggtgtttgagcttgtgggtgagccctccatatactgcaccagcaatgacgatcaagtgg |
| | | gcatctggagcggcccggcccctcagtgcattatacctggtggaggtggatcgggaggtggtggatctggtggaggtggatcgg |
| | | gaggtggtggatctggtggaggtggatcgatcttttgtccaagtcctccagttattcctaatgggagacacacaggaaaacctctgg |
| | | aagtctttccctttggaaaagcagtaaattacacatgcgacccccacccagacagagggacgagcttcgacctcattggagagag |
| | | caccatccgctgcacaagtgaccctcaagggaatggggtttggagcagccctgcccctcgctgtggaattctgggtcactgtcaa |
| | | gccccagatcattttctgtttgccaagttgaaaacccaaaccaatgcatctgactttcccattgggacatctttaaagtacgaatgccg |
| | | tcctgagtactacgggaggccattctctatcacatgtctagataacctggtctggtcaagtcccaaagatgtctgtaaacgtaaatcat |
| | | gtaaaactcctccagatccagtgaatggcatggtgcatgtgatcacagacatccaggttggatccagaatcaactattcttgtactac |
| | | agggcaccgactcattggtcactcatctgctgaatgtatcctctcaggcaatactgcccattggagcacgaagccgccaatttgtca |
| | | acgaattccttgtgggctacccccaaccatcgccaatggagatttcattagcaccaacagagagaattttcactatggatcagtggt |
| | | gacctaccgctgcaatcttggaagcagagggagaaaggtgtttgagcttgtgggtgagccctccatatactgcaccagcaatgac |
| | | gatcaagtgggcatctggagcggccccgcccctcagtgcattatatag |
| 86 | CR2CR1 | atttcttgtggctctcctccgcctatcctaaatggcaggattagttattattctacccccattgctgttggtacagtgataaggtacagtt |
| | #10 (C) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tctagtggaaaatggagtgctgtcccccccacatgtgaagaggcaaggtgtaaatctctaggaaggtttcccaatgggaaggtaa |
| | | aggagcctccaattctcagggttggtgtaactgcaaactttttctgtgatgaagggtataggctgcaaggcccaccttctagtaggtg |
| | | tgtaattgctggacagggagttgcttggaccaaaatgccagtatgtgaagaaatttttcaatgcaatgccccagaatggcttccattt |
| | | gccaggcctaccaacctaactgatgaatttgagtttcccattgggacatatctgaactatgaatgccgccctggttatagcggaaga |
| | | ccattttctatcatctgcctaaaaaactcagtctggactggtgctaaggacaggtgcagaaggaaatcatgtaggaatcctccagat |
| | | cctgtgaatggcatggtgcatgtgatcaaaggcatccagtttggatcccaaattaaatattcttgtactaaaggataccgcctcattgg |
| | | ttcctcatctgccacatgcatcatctcaggtgatactgtcatttgggataatgaaacacctatttgtgacagaattccttgtgggctacc |
| | | ccccaccatcaccaatggagatttcattagcaccaacagagagaattttcactatggatcagtggtgacctaccgctgcaatcctgg |
| | | aagcggagggagaaaggtgtttgagcttgtgggtgagccctccatatactgcaccagcaatgatgatcaagtgggcatctggagc |
| | | ggccccgcccctcagtgcattatacctggtggaggtggaagtggaggtggtggatctggtggaggtggatcaggaggtggtgg |
| | | atctggtggaggtggaagtggaggtggtggatctggtggaggtggatcaggaggtggtggatctggtggaggtggaagtggag |
| | | gtggtggatctggtggaggtggatcaggaggtggtggatctatcttttgtccaagtcctccagttattcctaatgggagacacacag |
| | | gaaaacctctggaagtctttccctttgggaaaacagtaaattacacatgtgacccccacccagacagagggactagctttgacctc |
| | | attggagagagcaccatccgctgcacaagtgaccctcaagggaatggggtttggagcagccctgcccctaggtgtggaattctg |
| | | ggtcactgtcaagccccagatcattttctgtttgccaagttgaaaacccaaaccaatgcatctgactttcccattgggacatctttaaa |
| | | gtatgaatgccgccctgagtactatgggaggccattctctatcacatgtctagataacctggtctggtcaagtcccaaagatgtctgt |
| | | aaaaggaaatcatgtaaaactcctccagatccagtgaatggcatggtgcatgtgatcacagacatccaggttggatccagaatcaa |
| | | ctattcttgtactactgggcaccgcctcattggtcactcatctgctgaatgtatcctcagtggcaatgctgcccattggagcactaagc |
| | | cgccaatttgtcaaaggattccttgtgggctaccccccaccattgccaatggagatttcattagcaccaacagagagaattttcacta |
| | | tggatcagtggtgacctaccgctgcaatcctggaagcggagggagaaaggtgtttgagcttgtgggtgagccctccatatactgc |
| | | accagcaatgatgatcaagtgggcatctggagcggcccagcccctcagtgcattatacctggtggaggtggatcaggaggtggt |
| | | ggatctggtggaggtggaagtggaggtggtggatctggtggaggtggatcaggaggtggtggatctggtggaggtggaagtgg |
| | | aggtggtggatctggtggaggtggatcaggaggtggtggatctggtggaggtggaagtggaggtggtggatctatcttttgtcca |
| | | agtcctccagttattcctaatgggagacacacaggaaaacctctggaagtctttccctttggaaaagcagtaaattacacatgtgacc |
| | | cccacccagacagagggactagctttgacctcattggagagagcaccatccgctgcacaagtgaccctcaagggaatggggttt |
| | | ggagcagccctgcccctagatgtggaattctgggtcactgtcaagccccagatcattttctgtttgccaagttgaaaacccaaacca |
| | | atgcatctgactttcccattgggacatctttaaagtatgaatgccgccctgagtactatgggaggccattctctatcacatgtctagata |
| | | acctggtctggtcaagtcccaaagatgtctgtaaaaggaaatcatgtaaaactcctccagatccagtgaatggcatggtgcatgtga |
| | | tcacagacatccaggttggatccagaatcaactattcttgtactactgggcaccgcctcattggtcactcatctgctgaatgtatcctct |
| | | caggcaatactgcccattggagcactaagccgccaatttgtcaaagaattccttgtgggctacccccaaccattgccaatggagatt |
| | | tcattagcaccaacagagagaattttcactatggatcagtggtgacctaccgctgcaatcttggaagcagagggagaaaggtgttt |
| | | gagcttgtgggtgagccctccatatactgcaccagcaatgatgatcaagtgggcatctggagcggccccgcccctcagtgcattat |
| | | atag |
| 87 | CR2CR1 | atttcttgtggctctcctccgcctatcctaaatggcaggattagttattattctacccccattgctgttggtacagtgataaggtacagtt |
| | #10 (D) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tctagtggaaaatggagtgctgtcccccccacatgtgaagaggcaaggtgtaaatctctaggaaggtttcccaatgggaaggtaa |
| | | aggagcctccaattctcagggttggtgtaactgcaaactttttctgtgatgaagggtataggctgcaaggcccaccttctagtaggtg |
| | | tgtaattgctggacagggagttgcttggaccaaaatgccagtatgtgaagaaatttttcaatgcaatgccccagaatggcttccattt |
| | | gccaggcctaccaacctaactgatgaatttgagtttcccattgggacatatctgaactatgaatgccgccctggttatagcggaaga |
| | | ccattttctatcatctgcctaaaaaactcagtctggactggtgctaaggacaggtgcagaaggaaatcatgtaggaatcctccagat |
| | | cctgtgaatggcatggtgcatgtgatcaaaggcatccagtttggatcccaaattaaatattcttgtactaaaggataccgcctcattgg |
| | | ttcctcatctgccacatgcatcatctcaggtgatactgtcatttgggataatgaaacacctatttgtgacagaattccttgtgggctacc |
| | | ccccaccatcaccaatggagatttcattagcaccaacagagagaattttcactatggatcagtggtgacctaccgctgcaatcctgg |
| | | aagcggagggagaaaggtgtttgagcttgtgggtgagccctccatatactgcaccagcaatgatgatcaagtgggcatctggagc |
| | | ggccccgcccctcagtgcattatacctggtggaggtggaagtggaggtggtggatctggtggaggtggatcaggaggtggtgg |
| | | atctggtggaggtggaagtatcttttgtccaagtcctccagttattcctaatgggagacacacaggaaaacctctggaagtctttccct |
| | | ttgggaaaacagtaaattacacatgtgacccccacccagacagagggactagctttgacctcattggagagagcaccatccgctg |
| | | cacaagtgaccctcaagggaatggggtttggagcagccctgcccctaggtgtggaattctgggtcactgtcaagccccagatcat |
| | | tttctgtttgccaagttgaaaacccaaaccaatgcatctgactttcccattgggacatctttaaagtatgaatgccgccctgagtactat |
| | | gggaggccattctctatcacatgtctagataacctggtctggtcaagtcccaaagatgtctgtaaaaggaaatcatgtaaaactcctc |
| | | cagatccagtgaatggcatggtgcatgtgatcacagacatccaggttggatccagaatcaactattcttgtactactgggcaccgcc |
| | | tcattggtcactcatctgctgaatgtatcctcagtggcaatgctgcccattggagcactaagccgccaatttgtcaaaggattccttgt |
| | | gggctaccccccaccattgccaatggagatttcattagcaccaacagagagaattttcactatggatcagtggtgacctaccgctgc |
| | | aatcctggaagcggagggagaaaggtgtttgagcttgtgggtgagccctccatatactgcaccagcaatgatgatcaagtgggca |
| | | tctggagcggcccagcccctcagtgcattatacctggtggaggtggatcaggaggtggtggatctggtggaggtggaagtggag |
| | | gtggtggatctggtggaggtggatcaatcttttgtccaagtcctccagttattcctaatgggagacacacaggaaaacctctggaag |
| | | tctttccctttggaaaagcagtaaattacacatgtgacccccacccagacagagggactagctttgacctcattggagagagcacc |
| | | atccgctgcacaagtgaccctcaagggaatggggtttggagcagccctgcccctagatgtggaattctgggtcactgtcaagccc |
| | | cagatcattttctgtttgccaagttgaaaacccaaaccaatgcatctgactttcccattgggacatctttaaagtatgaatgccgccctg |
| | | agtactatgggaggccattctctatcacatgtctagataacctggtctggtcaagtcccaaagatgtctgtaaaaggaaatcatgtaa |
| | | aactcctccagatccagtgaatggcatggtgcatgtgatcacagacatccaggttggatccagaatcaactattcttgtactactggg |
| | | caccgcctcattggtcactcatctgctgaatgtatcctctcaggcaatactgcccattggagcactaagccgccaatttgtcaaagaa |
| | | ttccttgtgggctacccccaaccattgccaatggagatttcattagcaccaacagagagaattttcactatggatcagtggtgaccta |
| | | ccgctgcaatcttggaagcagagggagaaaggtgtttgagcttgtgggtgagccctccatatactgcaccagcaatgatgatcaa |
| | | gtgggcatctggagcggccccgcccctcagtgcattatatag |
| 88 | CR2CR1 | atttcttgtggctctcctccgcctatcctaaatggccggattagttattattctacccccattgctgttggtaccgtgataaggtacagtt |
| | #11 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttggtggaggtggatcgggaggtggtggatc |
| | | tcaatgcaatgccccagaatggcttccatttgccaggcctaccaacctaactgatgaatttgagtttcccattgggacatatctgaact |
| | | atgaatgccgccctggttattccggaagaccgttttctatcatctgcctaaaaaactcagtctggactggtgctaaggacaggtgca |
| | | gacgtaaatcatgtcgtaatcctccagatcctgtgaatggcatggtgcatgtgatcaaaggcatccagttcggatcccaaattaaata |
| | | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctggtggaggtggatcgggaggtggtggatc |
| | | tggtggaggtggatcgggaggtggtggatctggtggaggtggatcgggaggtggtggatctggtggaggtggatcgggaggt |
| | | ggtggatctggtggaggtggatcgggaggtggtggatctggtggaggtggatcgggaggtggtggatctatcttttgtccaagtc |
| | | ctccagttattcctaatgggagacacacaggaaaacctctggaagtctttccctttgggaaaacagtaaattacacatgcgaccccc |
| | | acccagacagagggacgagcttcgacctcattggagagagcaccatccgctgcacaagtgaccctcaagggaatggggtttgg |
| | | agcagccctgcccctcgctgtggaattctgggtcactgtcaagccccagatcattttctgtttgccaagttgaaaacccaaaccaat |
| | | gcatctgactttcccattgggacatctttaaagtacgaatgccgtcctgagtactacgggaggccattctctatcacatgtctagataa |
| | | cctggtctggtcaagtcccaaagatgtctgtaaacgtaaatcatgtaaaactcctccagatccagtgaatggcatggtgcatgtgatc |
| | | acagacatccaggttggatccagaatcaactattcttgtactacagggcaccgactcattggtcactcatctgctgaatgtatcctctc |
| | | gggcaatgctgcccattggagcacgaagccgccaatttgtcaacgaattccttgtgggctaccccccaccatcgccaatggagatt |
| | | tcattagcaccaacagagagaattttcactatggatcagtggtgacctaccgctgcaatcctggaagcggagggagaaaggtgttt |
| | | gagcttgtgggtgagccctccatatactgcaccagcaatgacgatcaagtgggcatctggagcggcccggcccctcagtgcatta |
| | | tacctggtggaggtggatcgggaggtggtggatctggtggaggtggatcgggaggtggtggatctggtggaggtggatcggga |
| | | ggtggtggatctggtggaggtggatcgggaggtggtggatctggtggaggtggatcgggaggtggtggatctggtggaggtgg |
| | | atcgggaggtggtggatctatcttttgtccaagtcctccagttattcctaatgggagacacacaggaaaacctctggaagtctttccct |
| | | ttggaaaagcagtaaattacacatgcgacccccacccagacagagggacgagcttcgacctcattggagagagcaccatccgct |
| | | gcacaagtgaccctcaagggaatggggtttggagcagccctgcccctcgctgtggaattctgggtcactgtcaagccccagatca |
| | | ttttctgtttgccaagttgaaaacccaaaccaatgcatctgactttcccattgggacatctttaaagtacgaatgccgtcctgagtacta |
| | | cgggaggccattctctatcacatgtctagataacctggtctggtcaagtcccaaagatgtctgtaaacgtaaatcatgtaaaactcct |
| | | ccagatccagtgaatggcatggtgcatgtgatcacagacatccaggttggatccagaatcaactattcttgtactacagggcaccg |
| | | actcattggtcactcatctgctgaatgtatcctctcaggcaatactgcccattggagcacgaagccgccaatttgtcaacgaattcctt |
| | | gtgggctacccccaaccatcgccaatggagatttcattagcaccaacagagagaattttcactatggatcagtggtgacctaccgct |
| | | gcaatcttggaagcagagggagaaaggtgtttgagcttgtgggtgagccctccatatactgcaccagcaatgacgatcaagtggg |
| | | catctggagcggccccgcccctcagtgcattatatag |
| 89 | CR2CR1 | atttcttgtggctctcctccgcctatcctaaatggccggattagttattattctacccccattgctgttggtaccgtgataaggtacagtt |
| | #11 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttggtggaggtggatcgggaggtggtggatc |
| | | tcaatgcaatgccccagaatggcttccatttgccaggcctaccaacctaactgatgaatttgagtttcccattgggacatatctgaact |
| | | atgaatgccgccctggttattccggaagaccgttttctatcatctgcctaaaaaactcagtctggactggtgctaaggacaggtgca |
| | | gacgtaaatcatgtcgtaatcctccagatcctgtgaatggcatggtgcatgtgatcaaaggcatccagttcggatcccaaattaaata |
| | | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctggtggaggtggatcgggaggtggtggatc |
| | | tggtggaggtggatcgggaggtggtggatctggtggaggtggatcgatcttttgtccaagtcctccagttattcctaatgggagaca |
| | | cacaggaaaacctctggaagtctttccctttgggaaaacagtaaattacacatgcgacccccacccagacagagggacgagcttc |
| | | gacctcattggagagagcaccatccgctgcacaagtgaccctcaagggaatggggtttggagcagccctgcccctcgctgtgga |
| | | attctgggtcactgtcaagccccagatcattttctgtttgccaagttgaaaacccaaaccaatgcatctgactttcccattgggacatct |
| | | ttaaagtacgaatgccgtcctgagtactacgggaggccattctctatcacatgtctagataacctggtctggtcaagtcccaaagatg |
| | | tctgtaaacgtaaatcatgtaaaactcctccagatccagtgaatggcatggtgcatgtgatcacagacatccaggttggatccagaa |
| | | tcaactattcttgtactacagggcaccgactcattggtcactcatctgctgaatgtatcctctcgggcaatgctgcccattggagcac |
| | | gaagccgccaatttgtcaacgaattccttgtgggctaccccccaccatcgccaatggagatttcattagcaccaacagagagaattt |
| | | tcactatggatcagtggtgacctaccgctgcaatcctggaagcggagggagaaaggtgtttgagcttgtgggtgagccctccatat |
| | | actgcaccagcaatgacgatcaagtgggcatctggagcggcccggcccctcagtgcattatacctggtggaggtggatcggga |
| | | ggtggtggatctggtggaggtggatcgggaggtggtggatctggtggaggtggatcgatcttttgtccaagtcctccagttattcct |
| | | aatgggagacacacaggaaaacctctggaagtctttccctttggaaaagcagtaaattacacatgcgacccccacccagacaga |
| | | gggacgagcttcgacctcattggagagagcaccatccgctgcacaagtgaccctcaagggaatggggtttggagcagccctgc |
| | | ccctcgctgtggaattctgggtcactgtcaagccccagatcattttctgtttgccaagttgaaaacccaaaccaatgcatctgactttc |
| | | ccattgggacatctttaaagtacgaatgccgtcctgagtactacgggaggccattctctatcacatgtctagataacctggtctggtc |
| | | aagtcccaaagatgtctgtaaacgtaaatcatgtaaaactcctccagatccagtgaatggcatggtgcatgtgatcacagacatcca |
| | | ggttggatccagaatcaactattcttgtactacagggcaccgactcattggtcactcatctgctgaatgtatcctctcaggcaatactg |
| | | cccattggagcacgaagccgccaatttgtcaacgaattccttgtgggctacccccaaccatcgccaatggagatttcattagcacc |
| | | aacagagagaattttcactatggatcagtggtgacctaccgctgcaatcttggaagcagagggagaaaggtgtttgagcttgtggg |
| | | tgagccctccatatactgcaccagcaatgacgatcaagtgggcatctggagcggccccgcccctcagtgcattatatag |
| 90 | CR2CR1 | atttcttgtggctctcctccgcctatcctaaatggcaggattagttattattctacccccattgctgttggtacagtgataaggtacagtt |
| | #11 (C) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tctagtggaaaatggagtgctgtcccccccacatgtgaagaggcaaggtgtaaatctctaggaaggtttcccaatgggaaggtaa |
| | | aggagcctccaattctcagggttggtgtaactgcaaactttttctgtgatgaagggtataggctgcaaggcccaccttctagtaggtg |
| | | tgtaattgctggacagggagttgcttggaccaaaatgccagtatgtgaagaaatttttggtggaggtggatcaggaggtggtggat |
| | | ctcaatgcaatgccccagaatggcttccatttgccaggcctaccaacctaactgatgaatttgagtttcccattgggacatatctgaa |
| | | ctatgaatgccgccctggttatagcggaagaccattttctatcatctgcctaaaaaactcagtctggactggtgctaaggacaggtg |
| | | cagaaggaaatcatgtaggaatcctccagatcctgtgaatggcatggtgcatgtgatcaaaggcatccagtttggatcccaaattaa |
| | | atattcttgtactaaaggataccgcctcattggttccagttctgccacatgcatcatctcaggtgatactgtcatttgggataatgaaac |
| | | acctatttgtgacagaattccttgtgggctaccccccaccatcaccaatggagatttcattagcaccaacagagagaattttcactatg |
| | | gatcagtggtgacctaccgctgcaatcctggaagcggagggagaaaggtgtttgagcttgtgggtgagccctccatatactgcac |
| | | cagcaatgatgatcaagtgggcatctggagcggccccgcccctcagtgcattatacctggtggaggtggatcaggaggtggtgg |
| | | atctggtggaggtggaagtggaggtggtggatctggtggaggtggatcaggaggtggtggatctggtggaggtggaagtggag |
| | | gtggtggatctggtggaggtggatcaggaggtggtggatctggtggaggtggaagtggaggtggtggatctatcttttgtccaagt |
| | | cctccagttattcctaatgggagacacacaggaaaacctctggaagtctttccctttgggaaaacagtaaattacacatgtgacccc |
| | | cacccagacagagggactagctttgacctcattggagagagcaccatccgctgcacaagtgaccctcaagggaatggggtttgg |
| | | agcagccctgcccctaggtgtggaattctgggtcactgtcaagccccagatcattttctgtttgccaagttgaaaacccaaaccaat |
| | | gcatctgactttcccattgggacatctttaaagtatgaatgccgccctgagtactatgggaggccattctctatcacatgtctagataa |
| | | cctggtctggtcaagtcccaaagatgtctgtaaaaggaaatcatgtaaaactcctccagatccagtgaatggcatggtgcatgtgat |
| | | cacagacatccaggttggatccagaatcaactattcttgtactactgggcaccgcctcattggtcactcatctgctgaatgtatcctct |
| | | caggcaatgctgcccattggagcactaagccgccaatttgtcaaaggattccttgtgggctaccccccaccattgccaatggagat |
| | | ttcattagcaccaacagagagaattttcactatggatcagtggtgacctaccgctgcaatcctggaagcggagggagaaaggtgtt |
| | | tgagcttgtgggtgagccctccatatactgcaccagcaatgatgatcaagtgggcatctggagcggcccagcccctcagtgcatta |
| | | tacctggtggaggtggaagtggaggtggtggatctggtggaggtggatcaggaggtggtggatctggtggaggtggaagtgga |
| | | ggtggtggatctggtggaggtggatcaggaggtggtggatctggtggaggtggaagtggaggtggtggatctggtggaggtgg |
| | | atcaggaggtggtggatctatcttttgtccaagtcctccagttattcctaatgggagacacacaggaaaacctctggaagtctttccct |
| | | ttggaaaagcagtaaattacacatgtgacccccacccagacagagggactagctttgacctcattggagagagcaccatccgctg |
| | | cacaagtgaccctcaagggaatggggtttggagcagccctgcccctagatgtggaattctgggtcactgtcaagccccagatcatt |
| | | ttctgtttgccaagttgaaaacccaaaccaatgcatctgactttcccattgggacatctttaaagtatgaatgccgccctgagtactatg |
| | | ggaggccattctctatcacatgtctagataacctggtctggtcaagtcccaaagatgtctgtaaaaggaaatcatgtaaaactcctcc |
| | | agatccagtgaatggcatggtgcatgtgatcacagacatccaggttggatccagaatcaactattcttgtactactgggcaccgcct |
| | | cattggtcactcatctgctgaatgtatcctctcaggcaatactgcccattggagcactaagccgccaatttgtcaaagaattccttgtg |
| | | ggctacccccaaccattgccaatggagatttcattagcaccaacagagagaattttcactatggatcagtggtgacctaccgctgca |
| | | atcttggaagcagagggagaaaggtgtttgagcttgtgggtgagccctccatatactgcaccagcaatgatgatcaagtgggcatc |
| | | tggagcggccccgcccctcagtgcattatatag |
| 91 | CR2CR1 | atttcttgtggctctcctccgcctatcctaaatggcaggattagttattattctacccccattgctgttggtacagtgataaggtacagtt |
| | #11 (D) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tctagtggaaaatggagtgctgtcccccccacatgtgaagaggcaaggtgtaaatctctaggaaggtttcccaatgggaaggtaa |
| | | aggagcctccaattctcagggttggtgtaactgcaaactttttctgtgatgaagggtataggctgcaaggcccaccttctagtaggtg |
| | | tgtaattgctggacagggagttgcttggaccaaaatgccagtatgtgaagaaatttttggtggaggtggatcaggaggtggtggat |
| | | ctcaatgcaatgccccagaatggcttccatttgccaggcctaccaacctaactgatgaatttgagtttcccattgggacatatctgaa |
| | | ctatgaatgccgccctggttatagcggaagaccattttctatcatctgcctaaaaaactcagtctggactggtgctaaggacaggtg |
| | | cagaaggaaatcatgtaggaatcctccagatcctgtgaatggcatggtgcatgtgatcaaaggcatccagtttggatcccaaattaa |
| | | atattcttgtactaaaggataccgcctcattggttccagttctgccacatgcatcatctcaggtgatactgtcatttgggataatgaaac |
| | | acctatttgtgacagaattccttgtgggctaccccccaccatcaccaatggagatttcattagcaccaacagagagaattttcactatg |
| | | gatcagtggtgacctaccgctgcaatcctggaagcggagggagaaaggtgtttgagcttgtgggtgagccctccatatactgcac |
| | | cagcaatgatgatcaagtgggcatctggagcggccccgcccctcagtgcattatacctggtggaggtggatcaggaggtggtgg |
| | | atctggtggaggtggaagtggaggtggtggatctggtggaggtggatcaatcttttgtccaagtcctccagttattcctaatgggag |
| | | acacacaggaaaacctctggaagtctttccctttgggaaaacagtaaattacacatgtgacccccacccagacagagggactagc |
| | | tttgacctcattggagagagcaccatccgctgcacaagtgaccctcaagggaatggggtttggagcagccctgcccctaggtgtg |
| | | gaattctgggtcactgtcaagccccagatcattttctgtttgccaagttgaaaacccaaaccaatgcatctgactttcccattgggaca |
| | | tctttaaagtatgaatgccgccctgagtactatgggaggccattctctatcacatgtctagataacctggtctggtcaagtcccaaag |
| | | atgtctgtaaaaggaaatcatgtaaaactcctccagatccagtgaatggcatggtgcatgtgatcacagacatccaggttggatcca |
| | | gaatcaactattcttgtactactgggcaccgcctcattggtcactcatctgctgaatgtatcctctcaggcaatgctgcccattggagc |
| | | actaagccgccaatttgtcaaaggattccttgtgggctaccccccaccattgccaatggagatttcattagcaccaacagagagaat |
| | | tttcactatggatcagtggtgacctaccgctgcaatcctggaagcggagggagaaaggtgtttgagcttgtgggtgagccctccat |
| | | atactgcaccagcaatgatgatcaagtgggcatctggagcggcccagcccctcagtgcattatacctggtggaggtggaagtgg |
| | | aggtggtggatctggtggaggtggatcaggaggtggtggatctggtggaggtggaagtatcttttgtccaagtcctccagttattcc |
| | | taatgggagacacacaggaaaacctctggaagtctttccctttggaaaagcagtaaattacacatgtgacccccacccagacaga |
| | | gggactagctttgacctcattggagagagcaccatccgctgcacaagtgaccctcaagggaatggggtttggagcagccctgcc |
| | | cctagatgtggaattctgggtcactgtcaagccccagatcattttctgtttgccaagttgaaaacccaaaccaatgcatctgactttcc |
| | | cattgggacatctttaaagtatgaatgccgccctgagtactatgggaggccattctctatcacatgtctagataacctggtctggtcaa |
| | | gtcccaaagatgtctgtaaaaggaaatcatgtaaaactcctccagatccagtgaatggcatggtgcatgtgatcacagacatccag |
| | | gttggatccagaatcaactattcttgtactactgggcaccgcctcattggtcactcatctgctgaatgtatcctctcaggcaatactgc |
| | | ccattggagcactaagccgccaatttgtcaaagaattccttgtgggctacccccaaccattgccaatggagatttcattagcaccaa |
| | | cagagagaattttcactatggatcagtggtgacctaccgctgcaatcttggaagcagagggagaaaggtgtttgagcttgtgggtg |
| | | agccctccatatactgcaccagcaatgatgatcaagtgggcatctggagcggccccgcccctcagtgcattatatag |
| 92 | CR2CR1 | atttcttgtggctctcctccgcctatcctaaatggccggattagttattattctacccccattgctgttggtaccgtgataaggtacagtt |
| | #12 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctggtggaggtggatcgggaggtggtggatctggtggaggtggatcggga |
| | | ggtggtggatctggtggaggtggatcgggaggtggtggatctcaatgcaatgccccagaatggcttccatttgccaggcctacca |
| | | acctaactgatgaatttgagtttcccattgggacatatctgaactatgaatgccgccctggttattccggaagaccgttttctatcatct |
| | | gcctaaaaaactcagtctggactggtgctaaggacaggtgcagacgtaaatcatgtcgtaatcctccagatcctgtgaatggcatg |
| | | gtgcatgtgatcaaaggcatccagttcggatcccaaattaaatattcttgtactaaaggataccgactcattggttcctcgtctgccac |
| | | atgcatcatctcaggtgatactgtcatttgggataatgaaacacctatttgtgacagaattccttgtgggctaccccccaccatcacca |
| | | atggagatttcattagcaccaacagagagaattttcactatggatcagtggtgacctaccgctgcaatcctggaagcggagggaga |
| | | aaggtgtttgagcttgtgggtgagccctccatatactgcaccagcaatgacgatcaagtgggcatctggagcggccccgcccctc |
| | | agtgcattatacctaacaaatgcacgcctccaaatgtggaaaatggaatattggtatctgacaacagaagcttattttccttaaatgaa |
| | | gttgtggagtttaggtgtcagcctggctttgtcatgaaaggaccccgccgtgtgaagtgccaggccctgaacaaatgggagccgg |
| | | agctaccaagctgctccagggtatgtcagccacctccagatgtcctgcatgctgagcgtacccaaagggacaaggacaacttttc |
| | | acctgggcaggaagtgttctacagctgtgagcccggctacgacctcagaggggctgcgtctatgcgctgcacaccccagggag |
| | | actggagccctgcagcccccacatgtgaagtgatcttttgtccaagtcctccagttattcctaatgggagacacacaggaaaacctc |
| | | tggaagtctttccctttgggaaaacagtaaattacacatgcgacccccacccagacagagggacgagcttcgacctcattggaga |
| | | gagcaccatccgctgcacaagtgaccctcaagggaatggggtttggagcagccctgcccctcgctgtggaattctgggtcactgt |
| | | caagccccagatcattttctgtttgccaagttgaaaacccaaaccaatgcatctgactttcccattgggacatctttaaagtacgaatg |
| | | ccgtcctgagtactacgggaggccattctctatcacatgtctagataacctggtctggtcaagtcccaaagatgtctgtaaacgtaaa |
| | | tcatgtaaaactcctccagatccagtgaatggcatggtgcatgtgatcacagacatccaggttggatccagaatcaactattcttgta |
| | | ctacagggcaccgactcattggtcactcatctgctgaatgtatcctctcgggcaatgctgcccattggagcacgaagccgccaattt |
| | | gtcaacgaattccttgtgggctaccccccaccatcgccaatggagatttcattagcaccaacagagagaattttcactatggatcagt |
| | | ggtgacctaccgctgcaatcctggaagcggagggagaaaggtgtttgagcttgtgggtgagccctccatatactgcaccagcaat |
| | | gacgatcaagtgggcatctggagcggcccggcccctcagtgcattatacctaacaaatgcacgcctccaaatgtggaaaatgga |
| | | atattggtatctgacaacagaagcttattttccttaaatgaagttgtggagtttaggtgtcagcctggctttgtcatgaaaggaccccgc |
| | | cgtgtgaagtgccaggccctgaacaaatgggagccggagctaccaagctgctccagggtatgtcagccacctccagatgtcctg |
| | | catgctgagcgtacccaaagggacaaggacaacttttcacccgggcaggaagtgttctacagctgtgagcccggctatgacctca |
| | | gaggggctgcgtctatgcgctgcacaccccagggagactggagccctgcagcccccacatgtgaagtgatcttttgtccaagtcc |
| | | tccagttattcctaatgggagacacacaggaaaacctctggaagtctttccctttggaaaagcagtaaattacacatgcgaccccca |
| | | cccagacagagggacgagcttcgacctcattggagagagcaccatccgctgcacaagtgaccctcaagggaatggggtttgga |
| | | gcagccctgcccctcgctgtggaattctgggtcactgtcaagccccagatcattttctgtttgccaagttgaaaacccaaaccaatg |
| | | catctgactttcccattgggacatctttaaagtacgaatgccgtcctgagtactacgggaggccattctctatcacatgtctagataac |
| | | ctggtctggtcaagtcccaaagatgtctgtaaacgtaaatcatgtaaaactcctccagatccagtgaatggcatggtgcatgtgatca |
| | | cagacatccaggttggatccagaatcaactattcttgtactacagggcaccgactcattggtcactcatctgctgaatgtatcctctca |
| | | ggcaatactgcccattggagcacgaagccgccaatttgtcaacgaattccttgtgggctacccccaaccatcgccaatggagattt |
| | | cattagcaccaacagagagaattttcactatggatcagtggtgacctaccgctgcaatcttggaagcagagggagaaaggtgtttg |
| | | agcttgtgggtgagccctccatatactgcaccagcaatgacgatcaagtgggcatctggagcggccccgcccctcagtgcattat |
| | | atag |
| 93 | CR2CR1 | atttcttgtggctctcctccgcctatcctaaatggccggattagttattattctacccccattgctgttggtaccgtgataaggtacagtt |
| | #12 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctggtggaggtggatcgggaggtggtggatctggtggaggtggatcgcaa |
| | | tgcaatgccccagaatggcttccatttgccaggcctaccaacctaactgatgaatttgagtttcccattgggacatatctgaactatga |
| | | atgccgccctggttattccggaagaccgttttctatcatctgcctaaaaaactcagtctggactggtgctaaggacaggtgcagacg |
| | | taaatcatgtcgtaatcctccagatcctgtgaatggcatggtgcatgtgatcaaaggcatccagttcggatcccaaattaaatattctt |
| | | gtactaaaggataccgactcattggttcctcgtctgccacatgcatcatctcaggtgatactgtcatttgggataatgaaacacctattt |
| | | gtgacagaattccttgtgggctaccccccaccatcaccaatggagatttcattagcaccaacagagagaattttcactatggatcagt |
| | | ggtgacctaccgctgcaatcctggaagcggagggagaaaggtgtttgagcttgtgggtgagccctccatatactgcaccagcaat |
| | | gacgatcaagtgggcatctggagcggccccgcccctcagtgcattatacctaacaaatgcacgcctccaaatgtggaaaatgga |
| | | atattggtatctgacaacagaagcttattttccttaaatgaagttgtggagtttaggtgtcagcctggctttgtcatgaaaggaccccgc |
| | | cgtgtgaagtgccaggccctgaacaaatgggagccggagctaccaagctgctccagggtatgtcagccacctccagatgtcctg |
| | | catgctgagcgtacccaaagggacaaggacaacttttcacctgggcaggaagtgttctacagctgtgagcccggctacgacctca |
| | | gaggggctgcgtctatgcgctgcacaccccagggagactggagccctgcagcccccacatgtgaagtgatcttttgtccaagtcc |
| | | tccagttattcctaatgggagacacacaggaaaacctctggaagtctttccctttgggaaaacagtaaattacacatgcgaccccca |
| | | cccagacagagggacgagcttcgacctcattggagagagcaccatccgctgcacaagtgaccctcaagggaatggggtttgga |
| | | gcagccctgcccctcgctgtggaattctgggtcactgtcaagccccagatcattttctgtttgccaagttgaaaacccaaaccaatg |
| | | catctgactttcccattgggacatctttaaagtacgaatgccgtcctgagtactacgggaggccattctctatcacatgtctagataac |
| | | ctggtctggtcaagtcccaaagatgtctgtaaacgtaaatcatgtaaaactcctccagatccagtgaatggcatggtgcatgtgatca |
| | | cagacatccaggttggatccagaatcaactattcttgtactacagggcaccgactcattggtcactcatctgctgaatgtatcctctcg |
| | | ggcaatgctgcccattggagcacgaagccgccaatttgtcaacgaattccttgtgggctaccccccaccatcgccaatggagattt |
| | | cattagcaccaacagagagaattttcactatggatcagtggtgacctaccgctgcaatcctggaagcggagggagaaaggtgttt |
| | | gagcttgtgggtgagccctccatatactgcaccagcaatgacgatcaagtgggcatctggagcggcccggcccctcagtgcatta |
| | | tacctaacaaatgcacgcctccaaatgtggaaaatggaatattggtatctgacaacagaagcttattttccttaaatgaagttgtggag |
| | | tttaggtgtcagcctggctttgtcatgaaaggaccccgccgtgtgaagtgccaggccctgaacaaatgggagccggagctaccaa |
| | | gctgctccagggtatgtcagccacctccagatgtcctgcatgctgagcgtacccaaagggacaaggacaacttttcacccgggca |
| | | ggaagtgttctacagctgtgagcccggctatgacctcagaggggctgcgtctatgcgctgcacaccccagggagactggagccc |
| | | tgcagcccccacatgtgaagtgatcttttgtccaagtcctccagttattcctaatgggagacacacaggaaaacctctggaagtcttt |
| | | ccctttggaaaagcagtaaattacacatgcgacccccacccagacagagggacgagcttcgacctcattggagagagcaccatc |
| | | cgctgcacaagtgaccctcaagggaatggggtttggagcagccctgcccctcgctgtggaattctgggtcactgtcaagccccag |
| | | atcattttctgtttgccaagttgaaaacccaaaccaatgcatctgactttcccattgggacatctttaaagtacgaatgccgtcctgagt |
| | | actacgggaggccattctctatcacatgtctagataacctggtctggtcaagtcccaaagatgtctgtaaacgtaaatcatgtaaaac |
| | | tcctccagatccagtgaatggcatggtgcatgtgatcacagacatccaggttggatccagaatcaactattcttgtactacagggca |
| | | ccgactcattggtcactcatctgctgaatgtatcctctcaggcaatactgcccattggagcacgaagccgccaatttgtcaacgaatt |
| | | ccttgtgggctacccccaaccatcgccaatggagatttcattagcaccaacagagagaattttcactatggatcagtggtgacctac |
| | | cgctgcaatcttggaagcagagggagaaaggtgtttgagcttgtgggtgagccctccatatactgcaccagcaatgacgatcaag |
| | | tgggcatctggagcggccccgcccctcagtgcattatatag |
| 94 | CR2CR1 | atttcttgtggctctcctccgcctatcctaaatggcaggattagttattattctacccccattgctgttggtacagtgataaggtacagtt |
| | #12 (C) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctggtggaggtggaagtggaggtggtggatctggtggaggtggaagtgga |
| | | ggtggtggatctggtggaggtggaagtggaggtggtggatctcaatgcaatgccccagaatggcttccatttgccaggcctacca |
| | | acctaactgatgaatttgagtttcccattgggacatatctgaactatgaatgccgccctggttatagcggaagaccattttctatcatct |
| | | gcctaaaaaactcagtctggactggtgctaaggacaggtgcagaaggaaatcatgtaggaatcctccagatcctgtgaatggcat |
| | | ggtgcatgtgatcaaaggcatccagtttggatcccaaattaaatattcttgtactaaaggataccgcctcattggttccagttctgcca |
| | | catgcatcatctcaggtgatactgtcatttgggataatgaaacacctatttgtgacagaattccttgtgggctaccccccaccatcacc |
| | | aatggagatttcattagcaccaacagagagaattttcactatggatcagtggtgacctaccgctgcaatcctggaagcggagggag |
| | | aaaggtgtttgagcttgtgggtgagccctccatatactgcaccagcaatgatgatcaagtgggcatctggagcggccccgcccct |
| | | cagtgcattatacctaacaaatgcactcctccaaatgtggaaaatggaatattggtatctgacaacagaagcttattttccttaaatga |
| | | agttgtggagtttaggtgtcagcctggctttgtcatgaaaggaccccgcagggtgaagtgccaggccctgaacaaatgggagcca |
| | | gagctaccaagctgctccagggtatgtcagccacctccagatgtcctgcatgctgagcgcacccaaagggacaaggacaactttt |
| | | cacctgggcaggaagtgttctacagctgtgagcctggctatgacctcagaggggctgcatctatgcgctgcacaccccagggag |
| | | actggagccctgcagcccccacatgtgaagtgatcttttgtccaagtcctccagttattcctaatgggagacacacaggaaaacctc |
| | | tggaagtctttccctttgggaaaacagtaaattacacatgtgacccccacccagacagagggactagctttgacctcattggagag |
| | | agcaccatccgctgcacaagtgaccctcaagggaatggggtttggagcagccctgcccctaggtgtggaattctgggtcactgtc |
| | | aagccccagatcattttctgtttgccaagttgaaaacccaaaccaatgcatctgactttcccattgggacatctttaaagtatgaatgc |
| | | cgccctgagtactatgggaggccattctctatcacatgtctagataacctggtctggtcaagtcccaaagatgtctgtaaaaggaaat |
| | | catgtaaaactcctccagatccagtgaatggcatggtgcatgtgatcacagacatccaggttggatccagaatcaactattcttgtac |
| | | tactgggcaccgcctcattggtcactcatctgctgaatgtatcctcagtggcaatgctgcccattggagcactaagccgccaatttgt |
| | | caaaggattccttgtgggctaccccccaccattgccaatggagatttcattagcaccaacagagagaattttcactatggatcagtg |
| | | gtgacctaccgctgcaatcctggaagcggagggagaaaggtgtttgagcttgtgggtgagccctccatatactgcaccagcaatg |
| | | atgatcaagtgggcatctggagcggcccagcccctcagtgcattatacctaacaaatgcactcctccaaatgtggaaaatggaata |
| | | ttggtatctgacaacagaagcttattttccttaaatgaagttgtggagtttaggtgtcagcctggctttgtcatgaaaggaccccgcag |
| | | ggtgaagtgccaggccctgaacaaatgggagccagagctaccaagctgctccagggtatgtcagccacctccagatgtcctgca |
| | | tgctgagcgcacccaaagggacaaggacaacttttcacctgggcaggaagtgttctacagctgtgagcctggctatgacctcaga |
| | | ggggctgcatctatgcgctgcacaccccagggagactggagccctgcagcccccacatgtgaagtgatcttttgtccaagtcctc |
| | | cagttattcctaatgggagacacacaggaaaacctctggaagtctttccctttggaaaagcagtaaattacacatgtgacccccacc |
| | | cagacagagggactagctttgacctcattggagagagcaccatccgctgcacaagtgaccctcaagggaatggggtttggagca |
| | | gccctgcccctaggtgtggaattctgggtcactgtcaagccccagatcattttctgtttgccaagttgaaaacccaaaccaatgcatc |
| | | tgactttcccattgggacatctttaaagtatgaatgccgccctgagtactatgggaggccattctctatcacatgtctagataacctggt |
| | | ctggtcaagtcccaaagatgtctgtaaaagaaaatcatgtaaaactcctccagatccagtgaatggcatggtgcatgtgatcacaga |
| | | catccaggttggatccagaatcaactattcttgtactactgggcaccgcctcattggtcactcatctgctgaatgtatcctctcaggca |
| | | atactgcccattggagcactaagccgccaatttgtcaaaggattccttgtgggctacccccaaccattgccaatggagatttcattag |
| | | caccaacagagagaattttcactatggatcagtggtgacctaccgctgcaatcttggaagcagagggagaaaggtgtttgagcttg |
| | | tgggtgagccctccatatactgcaccagcaatgatgatcaagtgggcatctggagcggccccgcccctcagtgcattatatag |
| 95 | CR2CR1 | atttcttgtggctctcctccgcctatcctaaatggcaggattagttattattctacccccattgctgttggtacagtgataaggtacagtt |
| | #12 (D) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctggtggaggtggaagtggaggtggtggatctggtggaggtggaagtcaa |
| | | tgcaatgccccagaatggcttccatttgccaggcctaccaacctaactgatgaatttgagtttcccattgggacatatctgaactatga |
| | | atgccgccctggttatagcggaagaccattttctatcatctgcctaaaaaactcagtctggactggtgctaaggacaggtgcagaag |
| | | gaaatcatgtaggaatcctccagatcctgtgaatggcatggtgcatgtgatcaaaggcatccagtttggatcccaaattaaatattctt |
| | | gtactaaaggataccgcctcattggttccagttctgccacatgcatcatctcaggtgatactgtcatttgggataatgaaacacctattt |
| | | gtgacagaattccttgtgggctaccccccaccatcaccaatggagatttcattagcaccaacagagagaattttcactatggatcagt |
| | | ggtgacctaccgctgcaatcctggaagcggagggagaaaggtgtttgagcttgtgggtgagccctccatatactgcaccagcaat |
| | | gatgatcaagtgggcatctggagcggccccgcccctcagtgcattatacctaacaaatgcactcctccaaatgtggaaaatggaat |
| | | attggtatctgacaacagaagcttattttccttaaatgaagttgtggagtttaggtgtcagcctggctttgtcatgaaaggaccccgca |
| | | gggtgaagtgccaggccctgaacaaatgggagccagagctaccaagctgctccagggtatgtcagccacctccagatgtcctgc |
| | | atgctgagcgcacccaaagggacaaggacaacttttcacctgggcaggaagtgttctacagctgtgagcctggctatgacctcag |
| | | aggggctgcatctatgcgctgcacaccccagggagactggagccctgcagcccccacatgtgaagtgatcttttgtccaagtcct |
| | | ccagttattcctaatgggagacacacaggaaaacctctggaagtctttccctttgggaaaacagtaaattacacatgtgacccccac |
| | | ccagacagagggactagctttgacctcattggagagagcaccatccgctgcacaagtgaccctcaagggaatggggtttggagc |
| | | agccctgcccctaggtgtggaattctgggtcactgtcaagccccagatcattttctgtttgccaagttgaaaacccaaaccaatgcat |
| | | ctgactttcccattgggacatctttaaagtatgaatgccgccctgagtactatgggaggccattctctatcacatgtctagataacctg |
| | | gtctggtcaagtcccaaagatgtctgtaaaaggaaatcatgtaaaactcctccagatccagtgaatggcatggtgcatgtgatcaca |
| | | gacatccaggttggatccagaatcaactattcttgtactactgggcaccgcctcattggtcactcatctgctgaatgtatcctcagtgg |
| | | caatgctgcccattggagcactaagccgccaatttgtcaaaggattccttgtgggctaccccccaccattgccaatggagatttcatt |
| | | agcaccaacagagagaattttcactatggatcagtggtgacctaccgctgcaatcctggaagcggagggagaaaggtgtttgagc |
| | | ttgtgggtgagccctccatatactgcaccagcaatgatgatcaagtgggcatctggagcggcccagcccctcagtgcattatacct |
| | | aacaaatgcactcctccaaatgtggaaaatggaatattggtatctgacaacagaagcttattttccttaaatgaagttgtggagtttag |
| | | gtgtcagcctggctttgtcatgaaaggaccccgcagggtgaagtgccaggccctgaacaaatgggagccagagctaccaagct |
| | | gctccagggtatgtcagccacctccagatgtcctgcatgctgagcgcacccaaagggacaaggacaacttttcacctgggcagg |
| | | aagtgttctacagctgtgagcctggctatgacctcagaggggctgcatctatgcgctgcacaccccagggagactggagccctgc |
| | | agcccccacatgtgaagtgatcttttgtccaagtcctccagttattcctaatgggagacacacaggaaaacctctggaagtctttccc |
| | | tttggaaaagcagtaaattacacatgtgacccccacccagacagagggactagctttgacctcattggagagagcaccatccgct |
| | | gcacaagtgaccctcaagggaatggggtttggagcagccctgcccctaggtgtggaattctgggtcactgtcaagccccagatca |
| | | ttttctgtttgccaagttgaaaacccaaaccaatgcatctgactttcccattgggacatctttaaagtatgaatgccgccctgagtacta |
| | | tgggaggccattctctatcacatgtctagataacctggtctggtcaagtcccaaagatgtctgtaaaagaaaatcatgtaaaactcct |
| | | ccagatccagtgaatggcatggtgcatgtgatcacagacatccaggttggatccagaatcaactattcttgtactactgggcaccgc |
| | | ctcattggtcactcatctgctgaatgtatcctctcaggcaatactgcccattggagcactaagccgccaatttgtcaaaggattccttg |
| | | tgggctacccccaaccattgccaatggagatttcattagcaccaacagagagaattttcactatggatcagtggtgacctaccgctg |
| | | caatcttggaagcagagggagaaaggtgtttgagcttgtgggtgagccctccatatactgcaccagcaatgatgatcaagtgggc |
| | | atctggagcggccccgcccctcagtgcattatatag |
| 96 | CR2CR1 | atttcttgtggctctcctccgcctatcctaaatggccggattagttattattctacccccattgctgttggtaccgtgataaggtacagtt |
| | #1 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttattccggaagacc |
| | | gttttctatcatctgcctaaaaaactcagtctggactggtgctaaggacaggtgcagacgtaaatcatgtcgtaatcctccagatcct |
| | | gtgaatggcatggtgcatgtgatcaaaggcatccagttcggatcccaaattaaatattcttgtactaaaggataccgactcattggttc |
| | | ctcgtctgccacatgcatcatctcaggtgatactgtcatttgggataatgaaacacctatttgtgacagaattccttgtgggctacccc |
| | | ccaccatcaccaatggagatttcattagcaccaacagagagaattttcactatggatcagtggtgacctaccgctgcaatcctggaa |
| | | gcggagggagaaaggtgtttgagcttgtgggtgagccctccatatactgcaccagcaatgacgatcaagtgggcatctggagcg |
| | | gccccgcccctcagtgcattatacctaacaaatgcacgcctccaaatgtggaaaatggaatattggtatctgacaacagaagcttat |
| | | tttccttaaatgaagttgtggagtttaggtgtcagcctggctttgtcatgaaaggaccccgccgtgtgaagtgccaggccctgaaca |
| | | aatgggagccggagctaccaagctgctccagggtatgtcagccacctccagatgtcctgcatgctgagcgtacccaaagggaca |
| | | aggacaacttttcacctgggcaggaagtgttctacagctgtgagcccggctacgacctcagaggggctgcgtctatgcgctgcac |
| | | accccagggagactggagccctgcagcccccacatgtgaagtgaaatcctgtgatgacttcatgggccaacttcttaatggccgt |
| | | gtgctatttccagtaaatctccagcttggagcaaaagtggattttgtttgtgatgaaggatttcaattaaaaggcagctctgctagttac |
| | | tgtgtcttggctggaatggaaagcctttggaatagcagtgttccagtgtgtgaacaaatcttttgtccaagtcctccagttattcctaat |
| | | gggagacacacaggaaaacctctggaagtctttccctttgggaaaacagtaaattacacatgcgacccccacccagacagaggg |
| | | acgagcttcgacctcattggagagagcaccatccgctgcacaagtgaccctcaagggaatggggtttggagcagccctgcccct |
| | | cgctgtggaattctgggtcactgtcaagccccagatcattttctgtttgccaagttgaaaacccaaaccaatgcatctgactttcccatt |
| | | gggacatctttaaagtacgaatgccgtcctgagtactacgggaggccattctctatcacatgtctagataacctggtctggtcaagtc |
| | | ccaaagatgtctgtaaacgtaaatcatgtaaaactcctccagatccagtgaatggcatggtgcatgtgatcacagacatccaggttg |
| | | gatccagaatcaactattcttgtactacagggcaccgactcattggtcactcatctgctgaatgtatcctctcgggcaatgctgcccat |
| | | tggagcacgaagccgccaatttgtcaacgaattccttgtgggctaccccccaccatcgccaatggagatttcattagcaccaacag |
| | | agagaattttcactatggatcagtggtgacctaccgctgcaatcctggaagcggagggagaaaggtgtttgagcttgtgggtgagc |
| | | cctccatatactgcaccagcaatgacgatcaagtgggcatctggagcggcccggcccctcagtgcattatacctaacaaatag |
| 97 | CR2CR1 | atttcttgtggctctcctccgcctatcctaaatggcaggattagttattattctacccccattgctgttggtacagtgataaggtacagtt |
| | #1 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcatctggaaaatggagtgctgtcccccccacatgtgaagaggcaaggtgtaaatctctaggaaggtttcccaatgggaaggtaaa |
| | | ggagcctccaattctcagggttggtgtaactgcaaactttttctgtgatgaagggtataggctgcaaggcccaccttctagtagatgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttatagcggaagac |
| | | cattttctatcatctgcctaaaaaactcagtctggactggtgctaaggacaggtgcagaaggaaatcatgtagaaatcctccagatc |
| | | ctgtgaatggcatggtgcatgtgatcaaaggcatccagtttggatcccaaattaaatattcttgtactaaaggataccgcctcattggt |
| | | tcctcatctgccacatgcatcatctcaggtgatactgtcatttgggataatgaaacacctatttgtgacagaattccttgtgggctaccc |
| | | cccaccatcaccaatggagatttcattagcaccaacagagagaattttcactatggatcagtggtgacctaccgctgcaatcctgga |
| | | agcggagggagaaaggtgtttgagcttgtgggtgagccctccatatactgcaccagcaatgatgatcaagtgggcatctggagc |
| | | ggccccgcccctcagtgcattatacctaacaaatgcactcctccaaatgtggaaaatggaatattggtatctgacaacagaagctta |
| | | ttttccttaaatgaagttgtggagtttaggtgtcagcctggctttgtcatgaaaggaccccgcagggtgaagtgccaggccctgaac |
| | | aaatgggagccagagctaccaagctgctccagggtatgtcagccacctccagatgtcctgcatgctgagcgcacccaaaggga |
| | | caaggacaacttttcacctgggcaggaagtgttctacagctgtgagcctggctatgacctcagaggggctgcatctatgcgctgca |
| | | caccccagggagactggagccctgcagcccccacatgtgaagtgaaatcctgtgatgacttcatgggccaacttcttaatggcag |
| | | agtgctatttccagtaaatctccagcttggagcaaaagtggattttgtttgtgatgaaggatttcaattaaaaggcagctctgctagtta |
| | | ctgtgtcttggctggaatggaaagcctttggaatagcagtgttccagtgtgtgaacaaatcttttgtccaagtcctccagttattcctaa |
| | | tgggagacacacaggaaaacctctggaagtctttccctttgggaaaacagtaaattacacatgtgacccccacccagacagaggg |
| | | actagctttgacctcattggagagagcaccatccgctgcacaagtgaccctcaagggaatggggtttggagcagccctgccccta |
| | | ggtgtggaattctgggtcactgtcaagccccagatcattttctgtttgccaagttgaaaacccaaaccaatgcatctgactttcccatt |
| | | gggacatctttaaagtatgaatgccgccctgagtactatgggaggccattctctatcacatgtctagataacctggtctggtcaagtc |
| | | ccaaagatgtctgtaaaagaaaatcatgtaaaactcctccagatccagtgaatggcatggtgcatgtgatcacagacatccaggttg |
| | | gatccagaatcaactattcttgtacaactgggcaccgcctcattggtcactcatctgctgaatgtatcctctcaggcaatgctgcccat |
| | | tggagcactaagccgccaatttgtcaaaggattccttgtgggctaccccccaccattgccaatggagatttcattagcaccaacaga |
| | | gagaattttcactatggatcagtggtgacctaccgctgcaatcctggaagcggagggagaaaggtgtttgagcttgtgggtgagcc |
| | | ctccatatactgcaccagcaatgatgatcaagtgggcatctggagcggcccagcccctcagtgcattatacctaacaaatag |
| 98 | CR2CR1 | atttcttgtggctctcctccgcctatcctaaatggccggattagttattattctacccccattgctgttggtaccgtgataaggtacagtt |
| | #1 (C) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttattccggaagacc |
| | | gttttctatcatctgcctaaaaaactcagtctggactggtgctaaggacaggtgcagacgtaaatcatgtcgtaatcctccagatcct |
| | | gtgaatggcatggtgcatgtgatcaaaggcatccagttcggatcccaaattaaatattcttgtactaaaggataccgactcattggttc |
| | | ctcgtctgccacatgcatcatctcaggtgatactgtcatttgggataatgaaacacctatttgtgacagaattccttgtgggctacccc |
| | | ccaccatcaccaatggagatttcattagcaccaacagagagaattttcactatggatcagtggtgacctaccgctgcaatcctggaa |
| | | gcggagggagaaaggtgtttgagcttgtgggtgagccctccatatactgcaccagcaatgacgatcaagtgggcatctggagcg |
| | | gccccgcccctcagtgcattatacctaacaaatgcacgcctccaaatgtggaaaatggaatattggtatctgacaacagaagcttat |
| | | tttccttaaatgaagttgtggagtttaggtgtcagcctggctttgtcatgaaaggaccccgccgtgtgaagtgccaggccctgaaca |
| | | aatgggagccggagctaccaagctgctccagggtatgtcagccacctccagatgtcctgcatgctgagcgtacccaaagggaca |
| | | aggacaacttttcacctgggcaggaagtgttctacagctgtgagcccggctacgacctcagaggggctgcgtctatgcgctgcac |
| | | accccagggagactggagccctgcagcccccacatgtgaagtgaaatcctgtgatgacttcatgggccaacttcttaatggccgt |
| | | gtgctatttccagtaaatctccagcttggagcaaaagtggattttgtttgtgatgaaggatttcaattaaaaggcagctctgctagttac |
| | | tgtgtcttggctggaatggaaagcctttggaatagcagtgttccagtgtgtgaacaaatcttttgtccaagtcctccagttattcctaat |
| | | gggagacacacaggaaaacctctggaagtctttccctttgggaaaacagtaaattacacatgcgacccccacccagacagaggg |
| | | acgagcttcgacctcattggagagagcaccatccgctgcacaagtgaccctcaagggaatggggtttggagcagccctgcccct |
| | | cgctgtggaattctgggtcactgtcaagccccagatcattttctgtttgccaagttgaaaacccaaaccaatgcatctgactttcccatt |
| | | gggacatctttaaagtacgaatgccgtcctgagtactacgggaggccattctctatcacatgtctagataacctggtctggtcaagtc |
| | | ccaaagatgtctgtaaacgtaaatcatgtaaaactcctccagatccagtgaatggcatggtgcatgtgatcacagacatccaggttg |
| | | gatccagaatcaactattcttgtactacagggcaccgactcattggtcactcatctgctgaatgtatcctctcgggcaatgctgcccat |
| | | tggagcacgaagccgccaatttgtcaacgaattccttgtgggctaccccccaccatcgccaatggagatttcattagcaccaacag |
| | | agagaattttcactatggatcagtggtgacctaccgctgcaatcctggaagcggagggagaaaggtgtttgagcttgtgggtgagc |
| | | cctccatatactgcaccagcaatgacgatcaagtgggcatctggagcggcccggcccctcagtgcattatatag |
| 99 | CR2CR1 | atttcttgtggctctcctccgcctatcctaaatggccggattagttattattctacccccattgctgttggtaccgtgataaggtacagtt |
| | #2 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttggtggaggtggatcgggaggtggtggatc |
| | | tcaatgcaatgccccagaatggcttccatttgccaggcctaccaacctaactgatgaatttgagtttcccattgggacatatctgaact |
| | | atgaatgccgccctggttattccggaagaccgttttctatcatctgcctaaaaaactcagtctggactggtgctaaggacaggtgca |
| | | gacgtaaatcatgtcgtaatcctccagatcctgtgaatggcatggtgcatgtgatcaaaggcatccagttcggatcccaaattaaata |
| | | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccagggtatgtcagccacctccagatgt |
| | | cctgcatgctgagcgtacccaaagggacaaggacaacttttcacctgggcaggaagtgttctacagctgtgagcccggctacga |
| | | cctcagaggggctgcgtctatgcgctgcacaccccagggagactggagccctgcagcccccacatgtgaagtgaaatcctgtga |
| | | tgacttcatgggccaacttcttaatggccgtgtgctatttccagtaaatctccagcttggagcaaaagtggattttgtttgtgatgaagg |
| | | atttcaattaaaaggcagctctgctagttactgtgtcttggctggaatggaaagcctttggaatagcagtgttccagtgtgtgaacaaa |
| | | tcttttgtccaagtcctccagttattcctaatgggagacacacaggaaaacctctggaagtctttccctttgggaaaacagtaaattac |
| | | acatgcgacccccacccagacagagggacgagcttcgacctcattggagagagcaccatccgctgcacaagtgaccctcaagg |
| | | gaatggggtttggagcagccctgcccctcgctgtggaattctgggtcactgtcaagccccagatcattttctgtttgccaagttgaaa |
| | | acccaaaccaatgcatctgactttcccattgggacatctttaaagtacgaatgccgtcctgagtactacgggaggccattctctatca |
| | | catgtctagataacctggtctggtcaagtcccaaagatgtctgtaaacgtaaatcatgtaaaactcctccagatccagtgaatggcat |
| | | ggtgcatgtgatcacagacatccaggttggatccagaatcaactattcttgtactacagggcaccgactcattggtcactcatctgct |
| | | gaatgtatcctctcgggcaatgctgcccattggagcacgaagccgccaatttgtcaacgaattccttgtgggctaccccccaccatc |
| | | gccaatggagatttcattagcaccaacagagagaattttcactatggatcagtggtgacctaccgctgcaatcctggaagcggagg |
| | | gagaaaggtgtttgagcttgtgggtgagccctccatatactgcaccagcaatgacgatcaagtgggcatctggagcggcccggc |
| | | ccctcagtgcattatacctaacaaatag |
| 100 | CR2CR1 | atttcttgtggctctcctccgcctatcctaaatggcaggattagttattattctacccccattgctgttggtacagtgataaggtacagtt |
| | #2 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcatctggaaaatggagtgctgtcccccccacatgtgaagaggcaaggtgtaaatctctaggaaggtttcccaatgggaaggtaaa |
| | | ggagcctccaattctcagggttggtgtaactgcaaactttttctgtgatgaagggtataggctgcaaggcccaccttctagtagatgt |
| | | gtaattgctggacagggagttgcttggaccaaaatgccagtatgtgaagaaatttttggtggaggtggatcaggaggtggtggatc |
| | | tcaatgcaatgccccagaatggcttccatttgccaggcctaccaacctaactgatgaatttgagtttcccattgggacatatctgaact |
| | | atgaatgccgccctggttatagcggaagaccattttctatcatctgcctaaaaaactcagtctggactggtgctaaggacaggtgca |
| | | gaaggaaatcatgtagaaatcctccagatcctgtgaatggcatggtgcatgtgatcaaaggcatccagtttggatcccaaattaaat |
| | | attcttgtactaaaggataccgcctcattggttcctcatctgccacatgcatcatctcaggtgatactgtcatttgggataatgaaacac |
| | | ctatttgtgacagaattccttgtgggctaccccccaccatcaccaatggagatttcattagcaccaacagagagaattttcactatgg |
| | | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccagggtatgtcagccacctccagat |
| | | gtcctgcatgctgagcgcacccaaagggacaaggacaacttttcacctgggcaggaagtgttctacagctgtgagcctggctatg |
| | | acctcagaggggctgcatctatgcgctgcacaccccagggagactggagccctgcagcccccacatgtgaagtgaaatcctgtg |
| | | atgacttcatgggccaacttcttaatggcagagtgctatttccagtaaatctccagcttggagcaaaagtggattttgtttgtgatgaag |
| | | gatttcaattaaaaggcagctctgctagttactgtgtcttggctggaatggaaagcctttggaatagcagtgttccagtgtgtgaacaa |
| | | atcttttgtccaagtcctccagttattcctaatgggagacacacaggaaaacctctggaagtctttccctttgggaaaacagtaaatta |
| | | cacatgtgacccccacccagacagagggactagctttgacctcattggagagagcaccatccgctgcacaagtgaccctcaagg |
| | | gaatggggtttggagcagccctgcccctaggtgtggaattctgggtcactgtcaagccccagatcattttctgtttgccaagttgaaa |
| | | acccaaaccaatgcatctgactttcccattgggacatctttaaagtatgaatgccgccctgagtactatgggaggccattctctatca |
| | | catgtctagataacctggtctggtcaagtcccaaagatgtctgtaaaagaaaatcatgtaaaactcctccagatccagtgaatggcat |
| | | ggtgcatgtgatcacagacatccaggttggatccagaatcaactattcttgtacaactgggcaccgcctcattggtcactcatctgct |
| | | gaatgtatcctcagtggcaatgctgcccattggagcactaagccgccaatttgtcaaaggattccttgtgggctaccccccaccatt |
| | | gccaatggagatttcattagcaccaacagagagaattttcactatggatcagtggtgacctaccgctgcaatcctggaagcggagg |
| | | gagaaaggtgtttgagcttgtgggtgagccctccatatactgcaccagcaatgatgatcaagtgggcatctggagcggcccagcc |
| | | cctcagtgcattatacctaacaaatag |
| 101 | CR2CR1 | atttcttgtggctctcctccgcctatcctaaatggccggattagttattattctacccccattgctgttggtaccgtgataaggtacagtt |
| | #3 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttattccggaagacc |
| | | gttttctatcatctgcctaaaaaactcagtctggactggtgctaaggacaggtgcagacgtaaatcatgtcgtaatcctccagatcct |
| | | gtgaatggcatggtgcatgtgatcaaaggcatccagttcggatcccaaattaaatattcttgtactaaaggataccgactcattggttc |
| | | ctcgtctgccacatgcatcatctcaggtgatactgtcatttgggataatgaaacacctatttgtgacagaattccttgtgggctacccc |
| | | ccaccatcaccaatggagatttcattagcaccaacagagagaattttcactatggatcagtggtgacctaccgctgcaatcctggaa |
| | | gcggagggagaaaggtgtttgagcttgtgggtgagccctccatatactgcaccagcaatgacgatcaagtgggcatctggagcg |
| | | gccccgcccctcagtgcataattggtcactgtcaagccccagatcattttctgtttgccaagttgaaaacccaaaccaatgcatctga |
| | | ctttcccattgggacatctttaaagtacgaatgccgtcctgagtactacgggaggccattctctatcacatgtctagataacctggtct |
| | | ggtcaagtcccaaagatgtctgtaaacgtaaatcatgtaaaactcctccagatccagtgaatggcatggtgcatgtgatcacagaca |
| | | tccaggttggatccagaatcaactattcttgtactacagggcaccgactcattggtcactcatctgctgaatgtatcctctcgggcaat |
| | | gctgcccattggagcacgaagccgccaatttgtcaacgaattccttgtgggctaccccccaccatcgccaatggagatttcattagc |
| | | accaacagagagaattttcactatggatcagtggtgacctaccgctgcaatcctggaagcggagggagaaaggtgtttgagcttgt |
| | | gggtgagccctccatatactgcaccagcaatgacgatcaagtgggcatctggagcggcccggcccctcagtgcattatacctaac |
| | | aaaggtcactgtcaagccccagatcattttctgtttgccaagttgaaaacccaaaccaatgcatctgactttcccattgggacatcttt |
| | | aaagtacgaatgccgtcctgagtactacgggaggccattctctatcacatgtctagataacctggtctggtcaagtcccaaagatgt |
| | | ctgtaaacgtaaatcatgtaaaactcctccagatccagtgaatggcatggtgcatgtgatcacagacatccaggttggatccagaat |
| | | caactattcttgtactacagggcaccgactcattggtcactcatctgctgaatgtatcctctcaggcaatactgcccattggagcacg |
| | | aagccgccaatttgtcaacgaattccttgtgggctacccccaaccatcgccaatggagatttcattagcaccaacagagagaatttt |
| | | cactatggatcagtggtgacctaccgctgcaatcttggaagcagagggagaaaggtgtttgagcttgtgggtgagccctccatata |
| | | ctgcaccagcaatgacgatcaagtgggcatctggagcggccccgcccctcagtgcattatatag |
| 102 | CR2CR1 | atttcttgtggctctcctccgcctatcctaaatggcaggattagttattattctacccccattgctgttggtacagtgataaggtacagtt |
| | #3 (B) | gttcaggtaccttccgcctcattggagaaaaaagtctattatgcataactaaagacaaagtggatggaacctgggataaacctgctc |
| | 2 | ctaaatgtgaatatttcaataaatattcttcttgccctgagcccatagtaccaggaggatacaaaattagaggctctacaccctacaga |
| | CR2-CR1 | catggtgattctgtgacatttgcctgtaaaaccaacttctccatgaatggaaacaagtctgtttggtgtcaagcaaataatatgtgggg |
| | only | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tctagtggaaaatggagtgctgtcccccccacatgtgaagaggcaaggtgtaaatctctaggaaggtttcccaatgggaaggtaa |
| | | aggagcctccaattctcagggttggtgtaactgcaaactttttctgtgatgaagggtataggctgcaaggcccaccttctagtaggtg |
| | | tgtaattgctggacagggagttgcttggaccaaaatgccagtatgtgaagaaatttttcaatgcaatgccccagaatggcttccattt |
| | | gccaggcctaccaacctaactgatgaatttgagtttcccattgggacatatctgaactatgaatgccgccctggttatagcggaaga |
| | | ccattttctatcatctgcctaaaaaactcagtctggactggtgctaaggacaggtgcagaaggaaatcatgtagaaatcctccagat |
| | | cctgtgaatggcatggtgcatgtgatcaaaggcatccagtttggatcccaaattaaatattcttgtactaaaggataccgcctcattgg |
| | | ttccagttctgccacatgcatcatctcaggtgatactgtcatttgggataatgaaacacctatttgtgacagaattccttgtgggctacc |
| | | ccccaccatcaccaatggagatttcattagcaccaacagagagaattttcactatggatcagtggtgacctaccgctgcaatcctgg |
| | | aagcggagggagaaaggtgtttgagcttgtgggtgagccctccatatactgcaccagcaatgatgatcaagtgggcatctggagc |
| | | ggccccgcccctcagtgcataattggtcactgtcaagccccagatcattttctgtttgccaagttgaaaacccaaaccaatgcatctg |
| | | actttcccattgggacatctttaaagtatgaatgccgccctgagtactatgggaggccattctctatcacatgtctagataacctggtct |
| | | ggtcaagtcccaaagatgtctgtaaaaggaaatcatgtaaaactcctccagatccagtgaatggcatggtgcatgtgatcacagac |
| | | atccaggttggatccagaatcaactattcttgtacaactgggcaccgcctcattggtcactcatctgctgaatgtatcctcagtggcaa |
| | | tgctgcccattggagcactaagccgccaatttgtcaaagaattccttgtgggctaccccccaccattgccaatggagatttcattagc |
| | | accaacagagagaattttcactatggatcagtggtgacctaccgctgcaatcctggaagcggagggagaaaggtgtttgagcttgt |
| | | gggtgagccctccatatactgcaccagcaatgatgatcaagtgggcatctggagcggcccagcccctcagtgcattatacctaac |
| | | aaaggtcactgtcaagccccagatcattttctgtttgccaagttgaaaacccaaaccaatgcatctgactttcccattgggacatcttt |
| | | aaagtatgaatgccgccctgagtactatgggaggccattctctatcacatgtctagataacctggtctggtcaagtcccaaagatgtc |
| | | tgtaaaaggaaatcatgtaaaactcctccagatccagtgaatggcatggtgcatgtgatcacagacatccaggttggatccagaat |
| | | caactattcttgtacaactgggcaccgcctcattggtcactcatctgctgaatgtatcctctcaggcaatactgcccattggagcacta |
| | | agccgccaatttgtcaaagaattccttgtgggctacccccaaccattgccaatggagatttcattagcaccaacagagagaattttca |
| | | ctatggatcagtggtgacctaccgctgcaatcttggaagcagagggagaaaggtgtttgagcttgtgggtgagccctccatatact |
| | | gcaccagcaatgatgatcaagtgggcatctggagcggccccgcccctcagtgcattatatag |
| 103 | CR2CR1 | atttcttgtggctctcctccgcctatcctaaatggccggattagttattattctacccccattgctgttggtaccgtgataaggtacagtt |
| | #4 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttggtggaggtggatcgggaggtggtggatc |
| | | tcaatgcaatgccccagaatggcttccatttgccaggcctaccaacctaactgatgaatttgagtttcccattgggacatatctgaact |
| | | atgaatgccgccctggttattccggaagaccgttttctatcatctgcctaaaaaactcagtctggactggtgctaaggacaggtgca |
| | | gacgtaaatcatgtcgtaatcctccagatcctgtgaatggcatggtgcatgtgatcaaaggcatccagttcggatcccaaattaaata |
| | | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcataattggtcactgtcaagccccagatcattttctgtttgc |
| | | caagttgaaaacccaaaccaatgcatctgactttcccattgggacatctttaaagtacgaatgccgtcctgagtactacgggaggcc |
| | | attctctatcacatgtctagataacctggtctggtcaagtcccaaagatgtctgtaaacgtaaatcatgtaaaactcctccagatccag |
| | | tgaatggcatggtgcatgtgatcacagacatccaggttggatccagaatcaactattcttgtactacagggcaccgactcattggtc |
| | | actcatctgctgaatgtatcctctcgggcaatgctgcccattggagcacgaagccgccaatttgtcaacgaattccttgtgggctacc |
| | | ccccaccatcgccaatggagatttcattagcaccaacagagagaattttcactatggatcagtggtgacctaccgctgcaatcctgg |
| | | aagcggagggagaaaggtgtttgagcttgtgggtgagccctccatatactgcaccagcaatgacgatcaagtgggcatctggag |
| | | cggcccggcccctcagtgcattatacctaacaaaggtcactgtcaagccccagatcattttctgtttgccaagttgaaaacccaaac |
| | | caatgcatctgactttcccattgggacatctttaaagtacgaatgccgtcctgagtactacgggaggccattctctatcacatgtctag |
| | | ataacctggtctggtcaagtcccaaagatgtctgtaaacgtaaatcatgtaaaactcctccagatccagtgaatggcatggtgcatgt |
| | | gatcacagacatccaggttggatccagaatcaactattcttgtactacagggcaccgactcattggtcactcatctgctgaatgtatc |
| | | ctctcaggcaatactgcccattggagcacgaagccgccaatttgtcaacgaattccttgtgggctacccccaaccatcgccaatgg |
| | | agatttcattagcaccaacagagagaattttcactatggatcagtggtgacctaccgctgcaatcttggaagcagagggagaaagg |
| | | tgtttgagcttgtgggtgagccctccatatactgcaccagcaatgacgatcaagtgggcatctggagcggccccgcccctcagtg |
| | | cattatatag |
| 104 | CR2CR1 | atttcttgtggctctcctccgcctatcctaaatggcaggattagttattattctacccccattgctgttggtacagtgataaggtacagtt |
| | #4 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tctagtggaaaatggagtgctgtcccccccacatgtgaagaggcaaggtgtaaatctctaggaaggtttcccaatgggaaggtaa |
| | | aggagcctccaattctcagggttggtgtaactgcaaactttttctgtgatgaagggtataggctgcaaggcccaccttctagtaggtg |
| | | tgtaattgctggacagggagttgcttggaccaaaatgccagtatgtgaagaaatttttggtggaggtggaagtggaggtggtggat |
| | | ctcaatgcaatgccccagaatggcttccatttgccaggcctaccaacctaactgatgaatttgagtttcccattgggacatatctgaa |
| | | ctatgaatgccgccctggttatagcggaagaccattttctatcatctgcctaaaaaactcagtctggactggtgctaaggacaggtg |
| | | cagaaggaaatcatgtagaaatcctccagatcctgtgaatggcatggtgcatgtgatcaaaggcatccagtttggatcccaaattaa |
| | | atattcttgtactaaaggataccgcctcattggttccagttctgccacatgcatcatctcaggtgatactgtcatttgggataatgaaac |
| | | acctatttgtgacagaattccttgtgggctaccccccaccatcaccaatggagatttcattagcaccaacagagagaattttcactatg |
| | | gatcagtggtgacctaccgctgcaatcctggaagcggagggagaaaggtgtttgagcttgtgggtgagccctccatatactgcac |
| | | cagcaatgatgatcaagtgggcatctggagcggccccgcccctcagtgcataattggtcactgtcaagccccagatcattttctgtt |
| | | tgccaagttgaaaacccaaaccaatgcatctgactttcccattgggacatctttaaagtatgaatgccgccctgagtactatgggag |
| | | gccattctctatcacatgtctagataacctggtctggtcaagtcccaaagatgtctgtaaaaggaaatcatgtaaaactcctccagatc |
| | | cagtgaatggcatggtgcatgtgatcacagacatccaggttggatccagaatcaactattcttgtacaactgggcaccgcctcattg |
| | | gtcactcatctgctgaatgtatcctcagtggcaatgctgcccattggagcactaagccgccaatttgtcaaagaattccttgtgggct |
| | | accccccaccattgccaatggagatttcattagcaccaacagagagaattttcactatggatcagtggtgacctaccgctgcaatcc |
| | | tggaagcggagggagaaaggtgtttgagcttgtgggtgagccctccatatactgcaccagcaatgatgatcaagtgggcatctgg |
| | | agcggcccagcccctcagtgcattatacctaacaaaggtcactgtcaagccccagatcattttctgtttgccaagttgaaaacccaa |
| | | accaatgcatctgactttcccattgggacatctttaaagtatgaatgccgccctgagtactatgggaggccattctctatcacatgtct |
| | | agataacctggtctggtcaagtcccaaagatgtctgtaaaaggaaatcatgtaaaactcctccagatccagtgaatggcatggtgc |
| | | atgtgatcacagacatccaggttggatccagaatcaactattcttgtacaactgggcaccgcctcattggtcactcatctgctgaatgt |
| | | atcctctcaggcaatactgcccattggagcactaagccgccaatttgtcaaagaattccttgtgggctacccccaaccattgccaat |
| | | ggagatttcattagcaccaacagagagaattttcactatggatcagtggtgacctaccgctgcaatcttggaagcagagggagaaa |
| | | ggtgtttgagcttgtgggtgagccctccatatactgcaccagcaatgatgatcaagtgggcatctggagcggccccgcccctcagt |
| | | gcattatatag |
| 105 | CR2CR1 | atttcttgtggctctcctccgcctatcctaaatggccggattagttattattctacccccattgctgttggtaccgtgataaggtacagtt |
| | #5 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttattccggaagacc |
| | | gttttctatcatctgcctaaaaaactcagtctggactggtgctaaggacaggtgcagacgtaaatcatgtcgtaatcctccagatcct |
| | | gtgaatggcatggtgcatgtgatcaaaggcatccagttcggatcccaaattaaatattcttgtactaaaggataccgactcattggttc |
| | | ctcgtctgccacatgcatcatctcaggtgatactgtcatttgggataatgaaacacctatttgtgacagaattccttgtgggctacccc |
| | | ccaccatcaccaatggagatttcattagcaccaacagagagaattttcactatggatcagtggtgacctaccgctgcaatcctggaa |
| | | gcggagggagaaaggtgtttgagcttgtgggtgagccctccatatactgcaccagcaatgacgatcaagtgggcatctggagcg |
| | | gccccgcccctcagtgcattatacctaacaaatgcacgcctccaaatgtggaaaatggaatattggtatctgacaacagaagcttat |
| | | tttccttaaatgaagttgtggagtttaggtgtcagcctggctttgtcatgaaaggaccccgccgtgtgaagtgccaggccctgaaca |
| | | aatgggagccggagctaccaagctgctccagggtatgtcagccacctccagatgtcctgcatgctgagcgtacccaaagggaca |
| | | aggacaacttttcacctgggcaggaagtgttctacagctgtgagcccggctacgacctcagaggggctgcgtctatgcgctgcac |
| | | accccagggagactggagccctgcagcccccacatgtgaagtgaaatcctgtgatgacttcatgggccaacttcttaatggccgt |
| | | gtgctatttccagtaaatctccagcttggagcaaaagtggattttgtttgtgatgaaggatttcaattaaaaggcagctctgctagttac |
| | | tgtgtcttggctggaatggaaagcctttggaatagcagtgttccagtgtgtgaacaaatcttttgtccaagtcctccagttattcctaat |
| | | gggagacacacaggaaaacctctggaagtctttccctttgggaaaacagtaaattacacatgcgacccccacccagacagaggg |
| | | acgagcttcgacctcattggagagagcaccatccgctgcacaagtgaccctcaagggaatggggtttggagcagccctgcccct |
| | | cgctgtggaattctgggtcactgtcaagccccagatcattttctgtttgccaagttgaaaacccaaaccaatgcatctgactttcccatt |
| | | gggacatctttaaagtacgaatgccgtcctgagtactacgggaggccattctctatcacatgtctagataacctggtctggtcaagtc |
| | | ccaaagatgtctgtaaacgtaaatcatgtaaaactcctccagatccagtgaatggcatggtgcatgtgatcacagacatccaggttg |
| | | gatccagaatcaactattcttgtactacagggcaccgactcattggtcactcatctgctgaatgtatcctctcgggcaatgctgcccat |
| | | tggagcacgaagccgccaatttgtcaacgaattccttgtgggctaccccccaccatcgccaatggagatttcattagcaccaacag |
| | | agagaattttcactatggatcagtggtgacctaccgctgcaatcctggaagcggagggagaaaggtgtttgagcttgtgggtgagc |
| | | cctccatatactgcaccagcaatgacgatcaagtgggcatctggagcggcccggcccctcagtgcattatacctaacaaatgcac |
| | | gcctccaaatgtggaaaatggaatattggtatctgacaacagaagcttattttccttaaatgaagttgtggagtttaggtgtcagcctg |
| | | gctttgtcatgaaaggaccccgccgtgtgaagtgccaggccctgaacaaatgggagccggagctaccaagctgctccagggtat |
| | | gtcagccacctccagatgtcctgcatgctgagcgtacccaaagggacaaggacaacttttcacccgggcaggaagtgttctacag |
| | | ctgtgagcccggctatgacctcagaggggctgcgtctatgcgctgcacaccccagggagactggagccctgcagcccccacat |
| | | gtgaagtgaaatcctgtgatgacttcatgggccaacttcttaatggccgtgtgctatttccagtaaatctccagcttggagcaaaagt |
| | | ggattttgtttgtgatgaaggatttcaattaaaaggcagctctgctagttattgtgtcttggctggaatggaaagcctttggaatagcag |
| | | tgttccagtgtgtgaacaaatcttttgtccaagtcctccagttattcctaatgggagacacacaggaaaacctctggaagtctttccctt |
| | | tggaaaagcagtaaattacacatgcgacccccacccagacagagggacgagcttcgacctcattggagagagcaccatccgct |
| | | gcacaagtgaccctcaagggaatggggtttggagcagccctgcccctcgctgtggaatttag |
| 106 | CR2CR1 | atttcttgtggctctcctccgcctatcctaaatggcaggattagttattattctacccccattgctgttggtacagtgataaggtacagtt |
| | #5 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcatctggaaaatggagtgctgtcccccccacatgtgaagaggcaaggtgtaaatctctaggaaggtttcccaatgggaaggtaaa |
| | | ggagcctccaattctcagggttggtgtaactgcaaactttttctgtgatgaagggtataggctgcaaggcccaccttctagtaggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttatagcggaagac |
| | | cattttctatcatctgcctaaaaaactcagtctggactggtgctaaggacaggtgcagaagaaaatcatgtaggaatcctccagatc |
| | | ctgtgaatggcatggtgcatgtgatcaaaggcatccagtttggatcccaaattaaatattcttgtactaaaggataccgcctcattggt |
| | | tcctcatctgccacatgcatcatctcaggtgatactgtcatttgggataatgaaacacctatttgtgacagaattccttgtgggctaccc |
| | | cccaccatcaccaatggagatttcattagcaccaacagagagaattttcactatggatcagtggtgacctaccgctgcaatcctgga |
| | | agcggagggagaaaggtgtttgagcttgtgggtgagccctccatatactgcaccagcaatgatgatcaagtgggcatctggagc |
| | | ggccccgcccctcagtgcattatacctaacaaatgcactcctccaaatgtggaaaatggaatattggtatctgacaacagaagctta |
| | | ttttccttaaatgaagttgtggagtttaggtgtcagcctggctttgtcatgaaaggaccccgcagagtgaagtgccaggccctgaac |
| | | aaatgggagccagagctaccaagctgctccagggtatgtcagccacctccagatgtcctgcatgctgagcgcacccaaaggga |
| | | caaggacaacttttcacctgggcaggaagtgttctacagctgtgagcctggctatgacctcagaggggctgcatctatgcgctgca |
| | | caccccagggagactggagccctgcagcccccacatgtgaagtgaaatcctgtgatgacttcatgggccaacttcttaatggcag |
| | | ggtgctatttccagtaaatctccagcttggagcaaaagtggattttgtttgtgatgaaggatttcaattaaaaggcagctctgctagtta |
| | | ctgtgtcttggctggaatggaaagcctttggaatagcagtgttccagtgtgtgaacaaatcttttgtccaagtcctccagttattcctaa |
| | | tgggagacacacaggaaaacctctggaagtctttccctttgggaaaacagtaaattacacatgtgacccccacccagacagaggg |
| | | actagctttgacctcattggagagagcaccatccgctgcacaagtgaccctcaagggaatggggtttggagcagccctgccccta |
| | | gatgtggaattctgggtcactgtcaagccccagatcattttctgtttgccaagttgaaaacccaaaccaatgcatctgactttcccatt |
| | | gggacatctttaaagtatgaatgccgccctgagtactatgggaggccattctctatcacatgtctagataacctggtctggtcaagtc |
| | | ccaaagatgtctgtaaaaggaaatcatgtaaaactcctccagatccagtgaatggcatggtgcatgtgatcacagacatccaggtt |
| | | ggatccagaatcaactattcttgtacaactgggcaccgcctcattggtcactcatctgctgaatgtatcctctcaggcaatgctgccc |
| | | attggagcactaagccgccaatttgtcaaagaattccttgtgggctaccccccaccattgccaatggagatttcattagcaccaaca |
| | | gagagaattttcactatggatcagtggtgacctaccgctgcaatcctggaagcggagggagaaaggtgtttgagcttgtgggtga |
| | | gccctccatatactgcaccagcaatgatgatcaagtgggcatctggagcggcccagcccctcagtgcattatacctaacaaatgca |
| | | ctcctccaaatgtggaaaatggaatattggtatctgacaacagaagcttattttccttaaatgaagttgtggagtttaggtgtcagcctg |
| | | gctttgtcatgaaaggaccccgcagggtgaagtgccaggccctgaacaaatgggagccagagctaccaagctgctccagggta |
| | | tgtcagccacctccagatgtcctgcatgctgagcgcacccaaagggacaaggacaacttttcacctgggcaggaagtgttctaca |
| | | gctgtgagcctggctatgacctcagaggggctgcatctatgcgctgcacaccccagggagactggagccctgcagcccccacat |
| | | gtgaagtgaaatcctgtgatgacttcatgggccaacttcttaatggcagagtgctatttccagtaaatctccagcttggagcaaaagt |
| | | ggattttgtttgtgatgaaggatttcaattaaaaggcagctctgctagttattgtgtcttggctggaatggaaagcctttggaatagcag |
| | | tgttccagtgtgtgaacaaatcttttgtccaagtcctccagttattcctaatgggagacacacaggaaaacctctggaagtctttccctt |
| | | tggaaaagcagtaaattacacatgtgacccccacccagacagagggactagctttgacctcattggagagagcaccatccgctgc |
| | | acaagtgaccctcaagggaatggggtttggagcagccctgcccctaggtgtggaatttag |
| 107 | CR2CR1 | atttcttgtggctctcctccgcctatcctaaatggccggattagttattattctacccccattgctgttggtaccgtgataaggtacagtt |
| | #6 (A) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttggtggaggtggatcgggaggtggtggatc |
| | | tcaatgcaatgccccagaatggcttccatttgccaggcctaccaacctaactgatgaatttgagtttcccattgggacatatctgaact |
| | | atgaatgccgccctggttattccggaagaccgttttctatcatctgcctaaaaaactcagtctggactggtgctaaggacaggtgca |
| | | gacgtaaatcatgtcgtaatcctccagatcctgtgaatggcatggtgcatgtgatcaaaggcatccagttcggatcccaaattaaata |
| | | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccagggtatgtcagccacctccagatgt |
| | | cctgcatgctgagcgtacccaaagggacaaggacaacttttcacctgggcaggaagtgttctacagctgtgagcccggctacga |
| | | cctcagaggggctgcgtctatgcgctgcacaccccagggagactggagccctgcagcccccacatgtgaagtgaaatcctgtga |
| | | tgacttcatgggccaacttcttaatggccgtgtgctatttccagtaaatctccagcttggagcaaaagtggattttgtttgtgatgaagg |
| | | atttcaattaaaaggcagctctgctagttactgtgtcttggctggaatggaaagcctttggaatagcagtgttccagtgtgtgaacaaa |
| | | tcttttgtccaagtcctccagttattcctaatgggagacacacaggaaaacctctggaagtctttccctttgggaaaacagtaaattac |
| | | acatgcgacccccacccagacagagggacgagcttcgacctcattggagagagcaccatccgctgcacaagtgaccctcaagg |
| | | gaatggggtttggagcagccctgcccctcgctgtggaattctgggtcactgtcaagccccagatcattttctgtttgccaagttgaaa |
| | | acccaaaccaatgcatctgactttcccattgggacatctttaaagtacgaatgccgtcctgagtactacgggaggccattctctatca |
| | | catgtctagataacctggtctggtcaagtcccaaagatgtctgtaaacgtaaatcatgtaaaactcctccagatccagtgaatggcat |
| | | ggtgcatgtgatcacagacatccaggttggatccagaatcaactattcttgtactacagggcaccgactcattggtcactcatctgct |
| | | gaatgtatcctctcgggcaatgctgcccattggagcacgaagccgccaatttgtcaacgaattccttgtgggctaccccccaccatc |
| | | gccaatggagatttcattagcaccaacagagagaattttcactatggatcagtggtgacctaccgctgcaatcctggaagcggagg |
| | | gagaaaggtgtttgagcttgtgggtgagccctccatatactgcaccagcaatgacgatcaagtgggcatctggagcggcccggc |
| | | ccctcagtgcattatacctaacaaatgcacgcctccaaatgtggaaaatggaatattggtatctgacaacagaagcttattttccttaa |
| | | atgaagttgtggagtttaggtgtcagcctggctttgtcatgaaaggaccccgccgtgtgaagtgccaggccctgaacaaatggga |
| | | gccggagctaccaagctgctccagggtatgtcagccacctccagatgtcctgcatgctgagcgtacccaaagggacaaggaca |
| | | acttttcacccgggcaggaagtgttctacagctgtgagcccggctatgacctcagaggggctgcgtctatgcgctgcacacccca |
| | | gggagactggagccctgcagcccccacatgtgaagtgaaatcctgtgatgacttcatgggccaacttcttaatggccgtgtgctatt |
| | | tccagtaaatctccagcttggagcaaaagtggattttgtttgtgatgaaggatttcaattaaaaggcagctctgctagttattgtgtcttg |
| | | gctggaatggaaagcctttggaatagcagtgttccagtgtgtgaacaaatcttttgtccaagtcctccagttattcctaatgggagac |
| | | acacaggaaaacctctggaagtctttccctttggaaaagcagtaaattacacatgcgacccccacccagacagagggacgagctt |
| | | cgacctcattggagagagcaccatccgctgcacaagtgaccctcaagggaatggggtttggagcagccctgcccctcgctgtgg |
| | | aatttag |
| 108 | CR2CR1 | atttcttgtggctctcctccgcctatcctaaatggcaggattagttattattctacccccattgctgttggtacagtgataaggtacagtt |
| | #6 (B) | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctttggagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcatctggaaaatggagtgctgtcccccccacatgtgaagaggcaaggtgtaaatctctaggaaggtttcccaatgggaaggtaaa |
| | | ggagcctccaattctcagggttggtgtaactgcaaactttttctgtgatgaagggtataggctgcaaggcccaccttctagtaggtgt |
| | | gtaattgctggacagggagttgcttggaccaaaatgccagtatgtgaagaaatttttggtggaggtggatcaggaggtggtggatc |
| | | tcaatgcaatgccccagaatggcttccatttgccaggcctaccaacctaactgatgaatttgagtttcccattgggacatatctgaact |
| | | atgaatgccgccctggttatagcggaagaccattttctatcatctgcctaaaaaactcagtctggactggtgctaaggacaggtgca |
| | | gaagaaaatcatgtaggaatcctccagatcctgtgaatggcatggtgcatgtgatcaaaggcatccagtttggatcccaaattaaat |
| | | attcttgtactaaaggataccgcctcattggttcctcatctgccacatgcatcatctcaggtgatactgtcatttgggataatgaaacac |
| | | ctatttgtgacagaattccttgtgggctaccccccaccatcaccaatggagatttcattagcaccaacagagagaattttcactatgg |
| | | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagagtgaagtgccaggccctgaacaaatgggagccagagctaccaagctgctccagggtatgtcagccacctccagat |
| | | gtcctgcatgctgagcgcacccaaagggacaaggacaacttttcacctgggcaggaagtgttctacagctgtgagcctggctatg |
| | | acctcagaggggctgcatctatgcgctgcacaccccagggagactggagccctgcagcccccacatgtgaagtgaaatcctgtg |
| | | atgacttcatgggccaacttcttaatggcagggtgctatttccagtaaatctccagcttggagcaaaagtggattttgtttgtgatgaa |
| | | ggatttcaattaaaaggcagctctgctagttactgtgtcttggctggaatggaaagcctttggaatagcagtgttccagtgtgtgaac |
| | | aaatcttttgtccaagtcctccagttattcctaatgggagacacacaggaaaacctctggaagtctttccctttgggaaaacagtaaat |
| | | tacacatgtgacccccacccagacagagggactagctttgacctcattggagagagcaccatccgctgcacaagtgaccctcaa |
| | | gggaatggggtttggagcagccctgcccctagatgtggaattctgggtcactgtcaagccccagatcattttctgtttgccaagttga |
| | | aaacccaaaccaatgcatctgactttcccattgggacatctttaaagtatgaatgccgccctgagtactatgggaggccattctctat |
| | | cacatgtctagataacctggtctggtcaagtcccaaagatgtctgtaaaaggaaatcatgtaaaactcctccagatccagtgaatgg |
| | | catggtgcatgtgatcacagacatccaggttggatccagaatcaactattcttgtacaactgggcaccgcctcattggtcactcatct |
| | | gctgaatgtatcctctcaggcaatgctgcccattggagcactaagccgccaatttgtcaaagaattccttgtgggctaccccccacc |
| | | attgccaatggagatttcattagcaccaacagagagaattttcactatggatcagtggtgacctaccgctgcaatcctggaagcgga |
| | | gggagaaaggtgtttgagcttgtgggtgagccctccatatactgcaccagcaatgatgatcaagtgggcatctggagcggcccag |
| | | cccctcagtgcattatacctaacaaatgcactcctccaaatgtggaaaatggaatattggtatctgacaacagaagcttattttccttaa |
| | | atgaagttgtggagtttaggtgtcagcctggctttgtcatgaaaggaccccgcagggtgaagtgccaggccctgaacaaatggga |
| | | gccagagctaccaagctgctccagggtatgtcagccacctccagatgtcctgcatgctgagcgcacccaaagggacaaggaca |
| | | acttttcacctgggcaggaagtgttctacagctgtgagcctggctatgacctcagaggggctgcatctatgcgctgcacaccccag |
| | | ggagactggagccctgcagcccccacatgtgaagtgaaatcctgtgatgacttcatgggccaacttcttaatggcagagtgctattt |
| | | ccagtaaatctccagcttggagcaaaagtggattttgtttgtgatgaaggatttcaattaaaaggcagctctgctagttattgtgtcttg |
| | | gctggaatggaaagcctttggaatagcagtgttccagtgtgtgaacaaatcttttgtccaagtcctccagttattcctaatgggagac |
| | | acacaggaaaacctctggaagtctttccctttggaaaagcagtaaattacacatgtgacccccacccagacagagggactagcttt |
| | | gacctcattggagagagcaccatccgctgcacaagtgaccctcaagggaatggggtttggagcagccctgcccctaggtgtgga |
| | | atttag |
| 109 | CR2CR1 | atttcttgtggctctcctccgcctatcctaaatggccggattagttattattctacccccattgctgttggtaccgtgataaggtacagtt |
| | #13 | gttcaggtaccttccgcctcattggagaaaaaagtctattatgcataactaaagacaaagtggatggaacctgggataaacctgctc |
| | CR2-CR1 | ctaaatgtgaatatttcaataaatattcttcttgccctgagcccatagtaccaggaggatacaaaattagaggctctacaccctacaga |
| | only | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaatttttcaatgcaatgccccagaatggcttccatttg |
| | | ccaggcctaccaacctaactgatgaatttgagtttcccattgggacatatctgaactatgaatgccgccctggttattccggaagacc |
| | | gttttctatcatctgcctaaaaaactcagtctggactggtgctaaggacaggtgcagacgtaaatcatgtgtaatcctccagatcct |
| | | gtgaatggcatggtgcatgtgatcaaaggcatccagttcggatcccaaattaaatattcttgtactaaaggataccgactcattggttc |
| | | ctcgtctgccacatgcatcatctcaggtgatactgtcatttgggataatgaaacacctatttgtgacagaattccttgtgggctacccc |
| | | ccaccatcaccaatggagatttcattagcaccaacagagagaattttcactatggatcagtggtgacctaccgctgcaatcctggaa |
| | | gcggagggagaaaggtgtttgagcttgtgggtgagccctccatatactgcaccagcaatgacgatcaagtgggcatctggagcg |
| | | gccccgcccctcagtgcattatacctaacaaatgcacgcctccaaatgtggaaaatggaatattggtatctgacaacagaagcttat |
| | | tttccttaaatgaagttgtggagtttaggtgtcagcctggctttgtcatgaaaggaccccgccgtgtgaagtgccaggccctgaaca |
| | | aatgggagccggagctaccaagctgctccagggtatgtcagccacctccagatgtcctgcatgctgagcgtacccaaagggaca |
| | | aggacaacttttcacctgggcaggaagtgttctacagctgtgagcccggctacgacctcagaggggctgcgtctatgcgctgcac |
| | | accccagggagactggagccctgcagcccccacatgtgaagtgaaatcctgtgatgacttcatgggccaacttcttaatggccgt |
| | | gtgctatttccagtaaatctccagcttggagcaaaagtggattttgtttgtgatgaaggatttcaattaaaaggcagctctgctagttac |
| | | tgtgtcttggctggaatggaaagcctttggaatagcagtgttccagtgtgtgaacaaatcttttgtccaagtcctccagttattcctaat |
| | | gggagacacacaggaaaacctctggaagtctttccctttgggaaaacagtaaattacacatgcgacccccacccagacagaggg |
| | | acgagcttcgacctcattggagagagcaccatccgctgcacaagtgaccctcaagggaatggggtttggagcagccctgcccct |
| | | cgctgtggaattctgggtcactgtcaagccccagatcattttctgtttgccaagttgaaaacccaaaccaatgcatctgactttcccatt |
| | | gggacatctttaaagtacgaatgccgtcctgagtactacgggaggccattctctatcacatgtctagataacctggtctggtcaagtc |
| | | ccaaagatgtctgtaaacgtaaatcatgtaaaactcctccagatccagtgaatggcatggtgcatgtgatcacagacatccaggttg |
| | | gatccagaatcaactattcttgtactacagggcaccgactcattggtcactcatctgctgaatgtatcctctcgggcaatgctgcccat |
| | | tggagcacgaagccgccaatttgtcaacgaattccttgtgggctaccccccaccatcgccaatggagatttcattagcaccaacag |
| | | agagaattttcactatggatcagtggtgacctaccgctgcaatcctggaagcggagggagaaaggtgtttgagcttgtgggtgagc |
| | | cctccatatactgcaccagcaatgacgatcaagtgggcatctggagcggcccggcccctcagtgcattatacctaacaaatgcac |
| | | gcctccaaatgtggaaaatggaatattggtatctgacaacagaagcttattttccttaaatgaagttgtggagtttaggtgtcagcctg |
| | | gctttgtcatgaaaggaccccgccgtgtgaagtgccaggccctgaacaaatgggagccggagctaccaagctgctccagggtat |
| | | gtcagccacctccagatgtcctgcatgctgagcgtacccaaagggacaaggacaacttttcacccgggcaggaagtgttctacag |
| | | ctgtgagcccggctatgacctcagaggggctgcgtctatgcgctgcacaccccagggagactggagccctgcagcccccacat |
| | | gtgaagtgaaatcctgtgatgacttcatgggccaacttcttaatggccgtgtgctatttccagtaaatctccagcttggagcaaaagt |
| | | ggattttgtttgtgatgaaggatttcaattaaaaggcagctctgctagttattgtgtcttggctggaatggaaagcctttggaatagcag |
| | | tgttccagtgtgtgaacaaatcttttgtccaagtcctccagttattcctaatgggagacacacaggaaaacctctggaagtctttccctt |
| | | tggaaaagcagtaaattacacatgcgacccccacccagacagagggacgagcttcgacctcattggagagagcaccatccgct |
| | | gcacaagtgaccctcaagggaatggggtttggagcagccctgcccctcgctgtggaattctgggtcactgtcaagccccagatca |
| | | ttttctgtttgccaagttgaaaacccaaaccaatgcatctgactttcccattgggacatctttaaagtacgaatgccgtcctgagtacta |
| | | cgggaggccattctctatcacatgtctagataacctggtctggtcaagtcccaaagatgtctgtaaacgtaaatcatgtaaaactcct |
| | | ccagatccagtgaatggcatggtgcatgtgatcacagacatccaggttggatccagaatcaactattcttgtactacagggcaccg |
| | | actcattggtcactcatctgctgaatgtatcctctcaggcaatactgcccattggagcacgaagccgccaatttgtcaacgaattcctt |
| | | gtgggctacccccaaccatcgccaatggagatttcattagcaccaacagagagaattttcactatggatcagtggtgacctaccgct |
| | | gcaatcttggaagcagagggagaaaggtgtttgagcttgtgggtgagccctccatatactgcaccagcaatgacgatcaagtggg |
| | | catctggagcggccccgcccctcagtgcattatatag |
| 110 | CR2 | atttcttgtggctctcctccgcctatcctaaatggccggattagttattattctacccccattgctgttggtaccgtgataaggtacagtt |
| | SCR1-4 | gttcaggtaccttccgcctcattggagaaaaaagtctattatgcataactaaagacaaagtggatggaacctgggataaacctgctc |
| | #1 | ctaaatgtgaatatttcaataaatattcttcttgccctgagcccatagtaccaggaggatacaaaattagaggctctacaccctacaga |
| | | catggtgattctgtgacatttgcctgtaaaaccaacttctccatgaacggaaacaagtctgtttggtgtcaagcaaataatatgtgggg |
| | | gccgacacgactaccaacctgtgtaagtgttttccctctcgagtgtccagcacttcctatgatccacaatggacatcacacaagtga |
| | | gaatgttggctccattgctccaggattgtctgtgacttacagctgtgaatctggttacttgcttgttggagaaaagatcattaactgtttg |
| | | tcttcgggaaaatggagtgctgtcccccccacatgtgaagaggcacgctgtaaatctctaggacgatttcccaatgggaaggtaaa |
| | | ggagcctccaattctccgggttggtgtaactgcaaactttttctgtgatgaagggtatcgactgcaaggcccaccttctagtcggtgt |
| | | gtaattgctggacagggagttgcttggaccaaaatgccagtatgtgaagaaattttt |
| 111 | CR2 | atttcttgtggctctcctccgcctatcctaaatggcaggattagttattattctacccccattgctgttggtacagtgataaggtacagtt |
| | SCR1-4 | gttcaggtaccttccgcctcattggagaaaaaagtctattatgcataactaaagacaaagtggatggaacctgggataaacctgctc |
| | #2 | ctaaatgtgaatatttcaataaatattcttcttgccctgagcccatagtaccaggaggatacaaaattagaggctctacaccctacaga |
| | | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccctcttgagtgtccagcacttcctatgatccacaatggacatcacacaagtgag |
| | | aatgttggctccattgctccaggattgtctgtgacttacagctgtgaatctggttacttgcttgttggagaaaagatcattaactgtttgt |
| | | ctagtggaaaatggagtgctgtcccccccacatgtgaagaggcaaggtgtaaatctctaggaaggtttcccaatgggaaggtaaa |
| | | ggagcctccaattctcagggttggtgtaactgcaaactttttctgtgatgaagggtataggctgcaaggcccaccttctagtaggtgt |
| | | gtaattgctggacagggagttgcttggaccaaaatgccagtatgtgaagaaattttt |
| 112 | CR2 | atttcttgtggctctcctccgcctatcctaaatggcaggattagttattattctacccccattgctgttggtacagtgataaggtacagtt |
| | SCR1-2 | gttcaggtaccttccgcctcattggagaaaaaagtctattatgcataactaaagacaaagtggatggaacctgggataaacctgctc |
| | #1 | ctaaatgtgaatatttcaataaatattcttcttgccctgagcccatagtaccaggaggatacaaaattagaggctctacaccctacaga |
| | | catggtgattctgtgacatttgcctgtaaaaccaacttctccatgaatggaaacaagtctgtttggtgtcaagcaaataatatgtgggg |
| | | gccaacaaggctaccaacctgtgtaagtgttttccct |
| 113 | CR2 | atttcttgtggctctcctccgcctatcctaaatggccggattagttattattctacccccattgctgttggtaccgtgataaggtacagtt |
| | SCR1-2 | gttcaggtaccttccgcctcattggagaaaaaagtctattatgcataactaaagacaaagtggatggaacctgggataaacctgctc |
| | #2 | ctaaatgtgaatatttcaataaatattcttcttgccctgagcccatagtaccaggaggatacaaaattagaggctctacaccctacaga |
| | | catggtgattctgtgacatttgcctgtaaaaccaacttctccatgaacggaaacaagtctgtttggtgtcaagcaaataatatgtgggg |
| | | gccgacacgactaccaacctgtgtaagtgttttccct |
| 114 | CR1 | caatgcaatgccccagaatggcttccatttgccaggcctaccaacctaactgatgaatttgagtttcccattgggacatatctgaact |
| | SCR1-4, | atgaatgccgccctggttattccggaagaccgttttctatcatctgcctaaaaaactcagtctggactggtgctaaggacaggtgca |
| | 7-11, | gacgtaaatcatgtcgtaatcctccagatcctgtgaatggcatggtgcatgtgatcaaaggcatccagttcggatcccaaattaaata |
| | 14-17 | ttcttgtactaaaggataccgactcattggttcctcgtctgccacatgcatcatctcaggtgatactgtcatttgggataatgaaacacc |
| | (CR2CR1 | tatttgtgacagaattccttgtgggctaccccccaccatcaccaatggagatttcattagcaccaacagagagaattttcactatggat |
| | #7) A | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccaggatcttttgtccaagtcctccagtt |
| | | attcctaatgggagacacacaggaaaacctctggaagtctttccctttgggaaaacagtaaattacacatgcgacccccacccaga |
| | | cagagggacgagcttcgacctcattggagagagcaccatccgctgcacaagtgaccctcaagggaatggggtttggagcagcc |
| | | ctgcccctcgctgtggaattctgggtcactgtcaagccccagatcattttctgtttgccaagttgaaaacccaaaccaatgcatctga |
| | | ctttcccattgggacatctttaaagtacgaatgccgtcctgagtactacgggaggccattctctatcacatgtctagataacctggtct |
| | | ggtcaagtcccaaagatgtctgtaaacgtaaatcatgtaaaactcctccagatccagtgaatggcatggtgcatgtgatcacagaca |
| | | tccaggttggatccagaatcaactattcttgtactacagggcaccgactcattggtcactcatctgctgaatgtatcctctcgggcaat |
| | | gctgcccattggagcacgaagccgccaatttgtcaacgaattccttgtgggctaccccccaccatcgccaatggagatttcattagc |
| | | accaacagagagaattttcactatggatcagtggtgacctaccgctgcaatcctggaagcggagggagaaaggtgtttgagcttgt |
| | | gggtgagccctccatatactgcaccagcaatgacgatcaagtgggcatctggagcggcccggcccctcagtgcattatacctaac |
| | | aaatgcacgcctccaaatgtggaaaatggaatattggtatctgacaacagaagcttattttccttaaatgaagttgtggagtttaggtg |
| | | tcagcctggctttgtcatgaaaggaccccgccgtgtgaagtgccaggccctgaacaaatgggagccggagctaccaagctgctc |
| | | caggatcttttgtccaagtcctccagttattcctaatgggagacacacaggaaaacctctggaagtctttccctttggaaaagcagta |
| | | aattacacatgcgacccccacccagacagagggacgagcttcgacctcattggagagagcaccatccgctgcacaagtgaccct |
| | | caagggaatggggtttggagcagccctgcccctcgctgtggaattctgggtcactgtcaagccccagatcattttctgtttgccaag |
| | | ttgaaaacccaaaccaatgcatctgactttcccattgggacatctttaaagtacgaatgccgtcctgagtactacgggaggccattct |
| | | ctatcacatgtctagataacctggtctggtcaagtcccaaagatgtctgtaaacgtaaatcatgtaaaactcctccagatccagtgaat |
| | | ggcatggtgcatgtgatcacagacatccaggttggatccagaatcaactattcttgtactacagggcaccgactcattggtcactcat |
| | | ctgctgaatgtatcctctcaggcaatactgcccattggagcacgaagccgccaatttgtcaacgaattccttgtgggctacccccaa |
| | | ccatcgccaatggagatttcattagcaccaacagagagaattttcactatggatcagtggtgacctaccgctgcaatcttggaagca |
| | | gagggagaaaggtgtttgagcttgtgggtgagccctccatatactgcaccagcaatgacgatcaagtgggcatctggagcggcc |
| | | ccgcccctcagtgcattatatag |
| 115 | CR1 | caatgcaatgccccagaatggcttccatttgccaggcctaccaacctaactgatgaatttgagtttcccattgggacatatctgaact |
| | SCR1-4, | atgaatgccgccctggttatagcggaagaccattttctatcatctgcctaaaaaactcagtctggactggtgctaaggacaggtgca |
| | 7-11, | gaaggaaatcatgtaggaatcctccagatcctgtgaatggcatggtgcatgtgatcaaaggcatccagtttggatcccaaattaaat |
| | 14-17 | attcttgtactaaaggataccgcctcattggttcctcatctgccacatgcatcatctcaggtgatactgtcatttgggataatgaaacac |
| | (CR2CR1 | ctatttgtgacagaattccttgtgggctaccccccaccatcaccaatggagatttcattagcaccaacagagagaattttcactatgg |
| | #7) B | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccaggatcttttgtccaagtcctccagt |
| | | tattcctaatgggagacacacaggaaaacctctggaagtctttccctttgggaaaacagtaaattacacatgtgacccccacccaga |
| | | cagagggactagctttgacctcattggagagagcaccatccgctgcacaagtgaccctcaagggaatggggtttggagcagccc |
| | | tgcccctagatgtggaattctgggtcactgtcaagccccagatcattttctgtttgccaagttgaaaacccaaaccaatgcatctgact |
| | | ttcccattgggacatctttaaagtatgaatgccgccctgagtactatgggaggccattctctatcacatgtctagataacctggtctgg |
| | | tcaagtcccaaagatgtctgtaaaaggaaatcatgtaaaactcctccagatccagtgaatggcatggtgcatgtgatcacagacatc |
| | | caggttggatccagaatcaactattcttgtactactgggcaccgcctcattggtcactcatctgctgaatgtatcctcagtggcaatgc |
| | | tgcccattggagcactaagccgccaatttgtcaaagaattccttgtgggctaccccccaccattgccaatggagatttcattagcacc |
| | | aacagagagaattttcactatggatcagtggtgacctaccgctgcaatcctggaagcggagggagaaaggtgtttgagcttgtgg |
| | | gtgagccctccatatactgcaccagcaatgatgatcaagtgggcatctggagcggcccagcccctcagtgcattatacctaacaa |
| | | atgcactcctccaaatgtggaaaatggaatattggtatctgacaacagaagcttattttccttaaatgaagttgtggagtttaggtgtca |
| | | gcctggctttgtcatgaaaggaccccgcagggtgaagtgccaggccctgaacaaatgggagccagagctaccaagctgctcca |
| | | ggatcttttgtccaagtcctccagttattcctaatgggagacacacaggaaaacctctggaagtctttccctttggaaaagcagtaaat |
| | | tacacatgtgacccccacccagacagagggactagctttgacctcattggagagagcaccatccgctgcacaagtgaccctcaa |
| | | gggaatggggtttggagcagccctgcccctagatgtggaattctgggtcactgtcaagccccagatcattttctgtttgccaagttga |
| | | aaacccaaaccaatgcatctgactttcccattgggacatctttaaagtatgaatgccgccctgagtactatgggaggccattctctat |
| | | cacatgtctagataacctggtctggtcaagtcccaaagatgtctgtaaaaggaaatcatgtaaaactcctccagatccagtgaatgg |
| | | catggtgcatgtgatcacagacatccaggttggatccagaatcaactattcttgtactactgggcaccgcctcattggtcactcatct |
| | | gctgaatgtatcctctcaggcaatactgcccattggagcactaagccgccaatttgtcaaagaattccttgtgggctacccccaacc |
| | | attgccaatggagatttcattagcaccaacagagagaattttcactatggatcagtggtgacctaccgctgcaatcttggaagcaga |
| | | gggagaaaggtgtttgagcttgtgggtgagccctccatatactgcaccagcaatgatgatcaagtgggcatctggagcggccccg |
| | | cccctcagtgcattatatag |
| 116 | CR1 | caatgcaatgccccagaatggcttccatttgccaggcctaccaacctaactgatgaatttgagtttcccattgggacatatctgaact |
| | SCR1-4, | atgaatgccgccctggttatagcggaagaccattttctatcatctgcctaaaaaactcagtctggactggtgctaaggacaggtgca |
| | 7-11, | gaaggaaatcatgtaggaatcctccagatcctgtgaatggcatggtgcatgtgatcaaaggcatccagtttggatcccaaattaaat |
| | 14-17 | attcttgtactaaaggataccgcctcattggttccagttctgccacatgcatcatctcaggtgatactgtcatttgggataatgaaacac |
| | (CR2CR1 | ctatttgtgacagaattccttgtgggctaccccccaccatcaccaatggagatttcattagcaccaacagagagaattttcactatgg |
| | #7) C | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccaggatcttttgtccaagtcctccagt |
| | | tattcctaatgggagacacacaggaaaacctctggaagtctttccctttgggaaaacagtaaattacacatgtgacccccacccaga |
| | | cagagggactagctttgacctcattggagagagcaccatccgctgcacaagtgaccctcaagggaatggggtttggagcagccc |
| | | tgcccctagatgtggaattctgggtcactgtcaagccccagatcattttctgtttgccaagttgaaaacccaaaccaatgcatctgact |
| | | ttcccattgggacatctttaaagtatgaatgccgccctgagtactatgggaggccattctctatcacatgtctagataacctggtctgg |
| | | tcaagtcccaaagatgtctgtaaaaggaaatcatgtaaaactcctccagatccagtgaatggcatggtgcatgtgatcacagacatc |
| | | caggttggatccagaatcaactattcttgtactactgggcaccgcctcattggtcactcatctgctgaatgtatcctctcaggcaatgc |
| | | tgcccattggagcactaagccgccaatttgtcaaagaattccttgtgggctaccccccaccattgccaatggagatttcattagcacc |
| | | aacagagagaattttcactatggatcagtggtgacctaccgctgcaatcctggaagcggagggagaaaggtgtttgagcttgtgg |
| | | gtgagccctccatatactgcaccagcaatgatgatcaagtgggcatctggagcggcccagcccctcagtgcattatacctaacaa |
| | | atgcactcctccaaatgtggaaaatggaatattggtatctgacaacagaagcttattttccttaaatgaagttgtggagtttaggtgtca |
| | | gcctggctttgtcatgaaaggaccccgcagggtgaagtgccaggccctgaacaaatgggagccagagctaccaagctgctcca |
| | | ggatcttttgtccaagtcctccagttattcctaatgggagacacacaggaaaacctctggaagtctttccctttggaaaagcagtaaat |
| | | tacacatgtgacccccacccagacagagggactagctttgacctcattggagagagcaccatccgctgcacaagtgaccctcaa |
| | | gggaatggggtttggagcagccctgcccctagatgtggaattctgggtcactgtcaagccccagatcattttctgtttgccaagttga |
| | | aaacccaaaccaatgcatctgactttcccattgggacatctttaaagtatgaatgccgccctgagtactatgggaggccattctctat |
| | | cacatgtctagataacctggtctggtcaagtcccaaagatgtctgtaaaaggaaatcatgtaaaactcctccagatccagtgaatgg |
| | | catggtgcatgtgatcacagacatccaggttggatccagaatcaactattcttgtactactgggcaccgcctcattggtcactcatct |
| | | gctgaatgtatcctctcaggcaatactgcccattggagcactaagccgccaatttgtcaaagaattccttgtgggctacccccaacc |
| | | attgccaatggagatttcattagcaccaacagagagaattttcactatggatcagtggtgacctaccgctgcaatcttggaagcaga |
| | | gggagaaaggtgtttgagcttgtgggtgagccctccatatactgcaccagcaatgatgatcaagtgggcatctggagcggccccg |
| | | cccctcagtgcattatatag |
| 117 | CR1 | caatgcaatgccccagaatggcttccatttgccaggcctaccaacctaactgatgaatttgagtttcccattgggacatatctgaact |
| | SCR1-4- | atgaatgccgccctggttattccggaagaccgttttctatcatctgcctaaaaaactcagtctggactggtgctaaggacaggtgca |
| | linker- | gacgtaaatcatgtcgtaatcctccagatcctgtgaatggcatggtgcatgtgatcaaaggcatccagttcggatcccaaattaaata |
| | 7-11- | ttcttgtactaaaggataccgactcattggttcctcgtctgccacatgcatcatctcaggtgatactgtcatttgggataatgaaacacc |
| | linker- | tatttgtgacagaattccttgtgggctaccccccaccatcaccaatggagatttcattagcaccaacagagagaattttcactatggat |
| | 14-17 | cagtggtgacctaccgctgcaatcctggaagcggagggagaaaggtgtttgagcttgtgggtgagccctccatatactgcaccag |
| | (CR2CR1 | caatgacgatcaagtgggcatctggagcggccccgcccctcagtgcattatacctaacaaatgcacgcctccaaatgtggaaaat |
| | #9) A | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccaggggtggaggtggatcgggaggt |
| | | ggtggatctatcttttgtccaagtcctccagttattcctaatgggagacacacaggaaaacctctggaagtctttccctttgggaaaac |
| | | agtaaattacacatgcgacccccacccagacagagggacgagcttcgacctcattggagagagcaccatccgctgcacaagtg |
| | | accctcaagggaatggggtttggagcagccctgcccctcgctgtggaattctgggtcactgtcaagccccagatcattttctgtttg |
| | | ccaagttgaaaacccaaaccaatgcatctgactttcccattgggacatctttaaagtacgaatgccgtcctgagtactacgggaggc |
| | | cattctctatcacatgtctagataacctggtctggtcaagtcccaaagatgtctgtaaacgtaaatcatgtaaaactcctccagatcca |
| | | gtgaatggcatggtgcatgtgatcacagacatccaggttggatccagaatcaactattcttgtactacagggcaccgactcattggt |
| | | cactcatctgctgaatgtatcctctcgggcaatgctgcccattggagcacgaagccgccaatttgtcaacgaattccttgtgggctac |
| | | cccccaccatcgccaatggagatttcattagcaccaacagagagaattttcactatggatcagtggtgacctaccgctgcaatcctg |
| | | gaagcggagggagaaaggtgtttgagcttgtgggtgagccctccatatactgcaccagcaatgacgatcaagtgggcatctgga |
| | | gcggcccggcccctcagtgcattatacctaacaaatgcacgcctccaaatgtggaaaatggaatattggtatctgacaacagaag |
| | | cttattttccttaaatgaagttgtggagtttaggtgtcagcctggctttgtcatgaaaggaccccgccgtgtgaagtgccaggccctg |
| | | aacaaatgggagccggagctaccaagctgctccaggggtggaggtggatcgggaggtggtggatctatcttttgtccaagtcctc |
| | | cagttattcctaatgggagacacacaggaaaacctctggaagtctttccctttggaaaagcagtaaattacacatgcgacccccacc |
| | | cagacagagggacgagcttcgacctcattggagagagcaccatccgctgcacaagtgaccctcaagggaatggggtttggagc |
| | | agccctgcccctcgctgtggaattctgggtcactgtcaagccccagatcattttctgtttgccaagttgaaaacccaaaccaatgcat |
| | | ctgactttcccattgggacatctttaaagtacgaatgccgtcctgagtactacgggaggccattctctatcacatgtctagataacctg |
| | | gtctggtcaagtcccaaagatgtctgtaaacgtaaatcatgtaaaactcctccagatccagtgaatggcatggtgcatgtgatcaca |
| | | gacatccaggttggatccagaatcaactattcttgtactacagggcaccgactcattggtcactcatctgctgaatgtatcctctcagg |
| | | caatactgcccattggagcacgaagccgccaatttgtcaacgaattccttgtgggctacccccaaccatcgccaatggagatttcat |
| | | tagcaccaacagagagaattttcactatggatcagtggtgacctaccgctgcaatcttggaagcagagggagaaaggtgtttgag |
| | | cttgtgggtgagccctccatatactgcaccagcaatgacgatcaagtgggcatctggagcggccccgcccctcagtgcattatata |
| | | g |
| 118 | CR1 | caatgcaatgccccagaatggcttccatttgccaggcctaccaacctaactgatgaatttgagtttcccattgggacatatctgaact |
| | SCR1-4- | atgaatgccgccctggttatagcggaagaccattttctatcatctgcctaaaaaactcagtctggactggtgctaaggacaggtgca |
| | linker- | gaaggaaatcatgtaggaatcctccagatcctgtgaatggcatggtgcatgtgatcaaaggcatccagtttggatcccaaattaaat |
| | 7-11- | attcttgtactaaaggataccgcctcattggttccagttctgccacatgcatcatctcaggtgatactgtcatttgggataatgaaacac |
| | linker- | ctatttgtgacagaattccttgtgggctaccccccaccatcaccaatggagatttcattagcaccaacagagagaattttcactatgg |
| | 14-17 | atcagtggtgacctaccgctgcaatcctggaagcggagggagaaaggtgtttgagcttgtgggtgagccctccatatactgcacc |
| | (CR2CR1 | agcaatgatgatcaagtgggcatctggagcggccccgcccctcagtgcattatacctaacaaatgcactcctccaaatgtggaaa |
| | #9) B | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccaggggggaggtggatcaggag |
| | | gtggtggatctatcttttgtccaagtcctccagttattcctaatgggagacacacaggaaaacctctggaagtctttccctttgggaaa |
| | | acagtaaattacacatgtgacccccacccagacagagggactagctttgacctcattggagagagcaccatccgctgcacaagtg |
| | | accctcaagggaatggggtttggagcagccctgcccctagatgtggaattctgggtcactgtcaagccccagatcattttctgtttg |
| | | ccaagttgaaaacccaaaccaatgcatctgactttcccattgggacatctttaaagtatgaatgccgccctgagtactatgggaggc |
| | | cattctctatcacatgtctagataacctggtctggtcaagtcccaaagatgtctgtaaaaggaaatcatgtaaaactcctccagatcca |
| | | gtgaatggcatggtgcatgtgatcacagacatccaggttggatccagaatcaactattcttgtactactgggcaccgcctcattggtc |
| | | actcatctgctgaatgtatcctcagtggcaatgctgcccattggagcactaagccgccaatttgtcaaagaattccttgtgggctacc |
| | | ccccaccattgccaatggagatttcattagcaccaacagagagaattttcactatggatcagtggtgacctaccgctgcaatcctgg |
| | | aagcggagggagaaaggtgtttgagcttgtgggtgagccctccatatactgcaccagcaatgatgatcaagtgggcatctggagc |
| | | ggcccagcccctcagtgcattatacctaacaaatgcactcctccaaatgtggaaaatggaatattggtatctgacaacagaagctta |
| | | ttttccttaaatgaagttgtggagtttaggtgtcagcctggctttgtcatgaaaggaccccgcagggtgaagtgccaggccctgaac |
| | | aaatgggagccagagctaccaagctgctccaggggtggaggtggatcaggaggtggtggatctatcttttgtccaagtcctccag |
| | | ttattcctaatgggagacacacaggaaaacctctggaagtctttccctttggaaaagcagtaaattacacatgtgacccccacccag |
| | | acagagggactagctttgacctcattggagagagcaccatccgctgcacaagtgaccctcaagggaatggggtttggagcagcc |
| | | ctgcccctagatgtggaattctgggtcactgtcaagccccagatcattttctgtttgccaagttgaaaacccaaaccaatgcatctga |
| | | ctttcccattgggacatctttaaagtatgaatgccgccctgagtactatgggaggccattctctatcacatgtctagataacctggtctg |
| | | gtcaagtcccaaagatgtctgtaaaaggaaatcatgtaaaactcctccagatccagtgaatggcatggtgcatgtgatcacagacat |
| | | ccaggttggatccagaatcaactattcttgtactactgggcaccgcctcattggtcactcatctgctgaatgtatcctctcaggcaata |
| | | ctgcccattggagcactaagccgccaatttgtcaaagaattccttgtgggctacccccaaccattgccaatggagatttcattagcac |
| | | caacagagagaattttcactatggatcagtggtgacctaccgctgcaatcttggaagcagagggagaaaggtgtttgagcttgtgg |
| | | gtgagccctccatatactgcaccagcaatgatgatcaagtgggcatctggagcggccccgcccctcagtgcattatatag |
| 119 | CR1 | caatgcaatgccccagaatggcttccatttgccaggcctaccaacctaactgatgaatttgagtttcccattgggacatatctgaact |
| | SCR1-3- | atgaatgccgccctggttattccggaagaccgttttctatcatctgcctaaaaaactcagtctggactggtgctaaggacaggtgca |
| | linker- | gacgtaaatcatgtcgtaatcctccagatcctgtgaatggcatggtgcatgtgatcaaaggcatccagttcggatcccaaattaaata |
| | 7-10- | ttcttgtactaaaggataccgactcattggttcctcgtctgccacatgcatcatctcaggtgatactgtcatttgggataatgaaacacc |
| | linker | tatttgtgacagaattccttgtgggctaccccccaccatcaccaatggagatttcattagcaccaacagagagaattttcactatggat |
| | 14-17 | cagtggtgacctaccgctgcaatcctggaagcggagggagaaaggtgtttgagcttgtgggtgagccctccatatactgcaccag |
| | (CR2CR1 | caatgacgatcaagtgggcatctggagcggccccgcccctcagtgcattatacctggtggaggtggatcgggaggtggtggatc |
| | #10) | tggtggaggtggatcgggaggtggtggatctggtggaggtggatcgggaggtggtggatctggtggaggtggatcgggaggt |
| | A | ggtggatctggtggaggtggatcgggaggtggtggatctggtggaggtggatcgggaggtggtggatctatcttttgtccaagtc |
| | | ctccagttattcctaatgggagacacacaggaaaacctctggaagtctttccctttgggaaaacagtaaattacacatgcgaccccc |
| | | acccagacagagggacgagcttcgacctcattggagagagcaccatccgctgcacaagtgaccctcaagggaatggggtttgg |
| | | agcagccctgcccctcgctgtggaattctgggtcactgtcaagccccagatcattttctgtttgccaagttgaaaacccaaaccaat |
| | | gcatctgactttcccattgggacatctttaaagtacgaatgccgtcctgagtactacgggaggccattctctatcacatgtctagataa |
| | | cctggtctggtcaagtcccaaagatgtctgtaaacgtaaatcatgtaaaactcctccagatccagtgaatggcatggtgcatgtgatc |
| | | acagacatccaggttggatccagaatcaactattcttgtactacagggcaccgactcattggtcactcatctgctgaatgtatcctctc |
| | | gggcaatgctgcccattggagcacgaagccgccaatttgtcaacgaattccttgtgggctaccccccaccatcgccaatggagatt |
| | | tcattagcaccaacagagagaattttcactatggatcagtggtgacctaccgctgcaatcctggaagcggagggagaaaggtgttt |
| | | gagcttgtgggtgagccctccatatactgcaccagcaatgacgatcaagtgggcatctggagcggcccggcccctcagtgcatta |
| | | tacctggtggaggtggatcgggaggtggtggatctggtggaggtggatcgggaggtggtggatctggtggaggtggatcggga |
| | | ggtggtggatctggtggaggtggatcgggaggtggtggatctggtggaggtggatcgggaggtggtggatctggtggaggtgg |
| | | atcgggaggtggtggatctatcttttgtccaagtcctccagttattcctaatgggagacacacaggaaaacctctggaagtctttccct |
| | | ttggaaaagcagtaaattacacatgcgacccccacccagacagagggacgagcttcgacctcattggagagagcaccatccgct |
| | | gcacaagtgaccctcaagggaatggggtttggagcagccctgcccctcgctgtggaattctgggtcactgtcaagccccagatca |
| | | ttttctgtttgccaagttgaaaacccaaaccaatgcatctgactttcccattgggacatctttaaagtacgaatgccgtcctgagtacta |
| | | cgggaggccattctctatcacatgtctagataacctggtctggtcaagtcccaaagatgtctgtaaacgtaaatcatgtaaaactcct |
| | | ccagatccagtgaatggcatggtgcatgtgatcacagacatccaggttggatccagaatcaactattcttgtactacagggcaccg |
| | | actcattggtcactcatctgctgaatgtatcctctcaggcaatactgcccattggagcacgaagccgccaatttgtcaacgaattcctt |
| | | gtgggctacccccaaccatcgccaatggagatttcattagcaccaacagagagaattttcactatggatcagtggtgacctaccgct |
| | | gcaatcttggaagcagagggagaaaggtgtttgagcttgtgggtgagccctccatatactgcaccagcaatgacgatcaagtggg |
| | | catctggagcggccccgcccctcagtgcattatatag |
| 120 | CR1 | caatgcaatgccccagaatggcttccatttgccaggcctaccaacctaactgatgaatttgagtttcccattgggacatatctgaact |
| | SCR1-3- | atgaatgccgccctggttattccggaagaccgttttctatcatctgcctaaaaaactcagtctggactggtgctaaggacaggtgca |
| | linker- | gacgtaaatcatgtcgtaatcctccagatcctgtgaatggcatggtgcatgtgatcaaaggcatccagttcggatcccaaattaaata |
| | 7-10- | ttcttgtactaaaggataccgactcattggttcctcgtctgccacatgcatcatctcaggtgatactgtcatttgggataatgaaacacc |
| | linker | tatttgtgacagaattccttgtgggctaccccccaccatcaccaatggagatttcattagcaccaacagagagaattttcactatggat |
| | 14-17 | cagtggtgacctaccgctgcaatcctggaagcggagggagaaaggtgtttgagcttgtgggtgagccctccatatactgcaccag |
| | (CR2CR1 | caatgacgatcaagtgggcatctggagcggccccgcccctcagtgcattatacctggtggaggtggatcgggaggtggtggatc |
| | #10) | tggtggaggtggatcgggaggtggtggatctggtggaggtggatcgatcttttgtccaagtcctccagttattcctaatgggagaca |
| | B | cacaggaaaacctctggaagtctttccctttgggaaaacagtaaattacacatgcgacccccacccagacagagggacgagcttc |
| | | gacctcattggagagagcaccatccgctgcacaagtgaccctcaagggaatggggtttggagcagccctgcccctcgctgtgga |
| | | attctgggtcactgtcaagccccagatcattttctgtttgccaagttgaaaacccaaaccaatgcatctgactttcccattgggacatct |
| | | ttaaagtacgaatgccgtcctgagtactacgggaggccattctctatcacatgtctagataacctggtctggtcaagtcccaaagatg |
| | | tctgtaaacgtaaatcatgtaaaactcctccagatccagtgaatggcatggtgcatgtgatcacagacatccaggttggatccagaa |
| | | tcaactattcttgtactacagggcaccgactcattggtcactcatctgctgaatgtatcctctcgggcaatgctgcccattggagcac |
| | | gaagccgccaatttgtcaacgaattccttgtgggctaccccccaccatcgccaatggagatttcattagcaccaacagagagaattt |
| | | tcactatggatcagtggtgacctaccgctgcaatcctggaagcggagggagaaaggtgtttgagcttgtgggtgagccctccatat |
| | | actgcaccagcaatgacgatcaagtgggcatctggagcggcccggcccctcagtgcattatacctggtggaggtggatcggga |
| | | ggtggtggatctggtggaggtggatcgggaggtggtggatctggtggaggtggatcgatcttttgtccaagtcctccagttattcct |
| | | aatgggagacacacaggaaaacctctggaagtctttccctttggaaaagcagtaaattacacatgcgacccccacccagacaga |
| | | gggacgagcttcgacctcattggagagagcaccatccgctgcacaagtgaccctcaagggaatggggtttggagcagccctgc |
| | | ccctcgctgtggaattctgggtcactgtcaagccccagatcattttctgtttgccaagttgaaaacccaaaccaatgcatctgactttc |
| | | ccattgggacatctttaaagtacgaatgccgtcctgagtactacgggaggccattctctatcacatgtctagataacctggtctggtc |
| | | aagtcccaaagatgtctgtaaacgtaaatcatgtaaaactcctccagatccagtgaatggcatggtgcatgtgatcacagacatcca |
| | | ggttggatccagaatcaactattcttgtactacagggcaccgactcattggtcactcatctgctgaatgtatcctctcaggcaatactg |
| | | cccattggagcacgaagccgccaatttgtcaacgaattccttgtgggctacccccaaccatcgccaatggagatttcattagcacc |
| | | aacagagagaattttcactatggatcagtggtgacctaccgctgcaatcttggaagcagagggagaaaggtgtttgagcttgtggg |
| | | tgagccctccatatactgcaccagcaatgacgatcaagtgggcatctggagcggccccgcccctcagtgcattatatag |
| 121 | CR1 | caatgcaatgccccagaatggcttccatttgccaggcctaccaacctaactgatgaatttgagtttcccattgggacatatctgaact |
| | SCR1-3- | atgaatgccgccctggttatagcggaagaccattttctatcatctgcctaaaaaactcagtctggactggtgctaaggacaggtgca |
| | linker- | gaaggaaatcatgtaggaatcctccagatcctgtgaatggcatggtgcatgtgatcaaaggcatccagtttggatcccaaattaaat |
| | 7-10- | attcttgtactaaaggataccgcctcattggttcctcatctgccacatgcatcatctcaggtgatactgtcatttgggataatgaaacac |
| | linker | ctatttgtgacagaattccttgtgggctaccccccaccatcaccaatggagatttcattagcaccaacagagagaattttcactatgg |
| | 14-17 | atcagtggtgacctaccgctgcaatcctggaagcggagggagaaaggtgtttgagcttgtgggtgagccctccatatactgcacc |
| | (CR2CR1 | agcaatgatgatcaagtgggcatctggagcggccccgcccctcagtgcattatacctggtggaggtggaagtggaggtggtgga |
| | #10) | tctggtggaggtggatcaggaggtggtggatctggtggaggtggaagtggaggtggtggatctggtggaggtggatcaggagg |
| | C | tggtggatctggtggaggtggaagtggaggtggtggatctggtggaggtggatcaggaggtggtggatctatcttttgtccaagtc |
| | | ctccagttattcctaatgggagacacacaggaaaacctctggaagtctttccctttgggaaaacagtaaattacacatgtgaccccc |
| | | acccagacagagggactagctttgacctcattggagagagcaccatccgctgcacaagtgaccctcaagggaatggggtttgga |
| | | gcagccctgcccctaggtgtggaattctgggtcactgtcaagccccagatcattttctgtttgccaagttgaaaacccaaaccaatg |
| | | catctgactttcccattgggacatctttaaagtatgaatgccgccctgagtactatgggaggccattctctatcacatgtctagataac |
| | | ctggtctggtcaagtcccaaagatgtctgtaaaaggaaatcatgtaaaactcctccagatccagtgaatggcatggtgcatgtgatc |
| | | acagacatccaggttggatccagaatcaactattcttgtactactgggcaccgcctcattggtcactcatctgctgaatgtatcctcag |
| | | tggcaatgctgcccattggagcactaagccgccaatttgtcaaaggattccttgtgggctaccccccaccattgccaatggagattt |
| | | cattagcaccaacagagagaattttcactatggatcagtggtgacctaccgctgcaatcctggaagcggagggagaaaggtgttt |
| | | gagcttgtgggtgagccctccatatactgcaccagcaatgatgatcaagtgggcatctggagcggcccagcccctcagtgcattat |
| | | acctggtggaggtggatcaggaggtggtggatctggtggaggtggaagtggaggtggtggatctggtggaggtggatcaggag |
| | | gtggtggatctggtggaggtggaagtggaggtggtggatctggtggaggtggatcaggaggtggtggatctggtggaggtgga |
| | | agtggaggtggtggatctatcttttgtccaagtcctccagttattcctaatgggagacacacaggaaaacctctggaagtctttccctt |
| | | tggaaaagcagtaaattacacatgtgacccccacccagacagagggactagctttgacctcattggagagagcaccatccgctgc |
| | | acaagtgaccctcaagggaatggggtttggagcagccctgcccctagatgtggaattctgggtcactgtcaagccccagatcattt |
| | | tctgtttgccaagttgaaaacccaaaccaatgcatctgactttcccattgggacatctttaaagtatgaatgccgccctgagtactatg |
| | | ggaggccattctctatcacatgtctagataacctggtctggtcaagtcccaaagatgtctgtaaaaggaaatcatgtaaaactcctcc |
| | | agatccagtgaatggcatggtgcatgtgatcacagacatccaggttggatccagaatcaactattcttgtactactgggcaccgcct |
| | | cattggtcactcatctgctgaatgtatcctctcaggcaatactgcccattggagcactaagccgccaatttgtcaaagaattccttgtg |
| | | ggctacccccaaccattgccaatggagatttcattagcaccaacagagagaattttcactatggatcagtggtgacctaccgctgca |
| | | atcttggaagcagagggagaaaggtgtttgagcttgtgggtgagccctccatatactgcaccagcaatgatgatcaagtgggcatc |
| | | tggagcggccccgcccctcagtgcattatatag |
| 122 | CR1 | caatgcaatgccccagaatggcttccatttgccaggcctaccaacctaactgatgaatttgagtttcccattgggacatatctgaact |
| | SCR1-3- | atgaatgccgccctggttatagcggaagaccattttctatcatctgcctaaaaaactcagtctggactggtgctaaggacaggtgca |
| | linker- | gaaggaaatcatgtaggaatcctccagatcctgtgaatggcatggtgcatgtgatcaaaggcatccagtttggatcccaaattaaat |
| | 7-10- | attcttgtactaaaggataccgcctcattggttcctcatctgccacatgcatcatctcaggtgatactgtcatttgggataatgaaacac |
| | linker | ctatttgtgacagaattccttgtgggctaccccccaccatcaccaatggagatttcattagcaccaacagagagaattttcactatgg |
| | 14-17 | atcagtggtgacctaccgctgcaatcctggaagcggagggagaaaggtgtttgagcttgtgggtgagccctccatatactgcacc |
| | (CR2CR1 | agcaatgatgatcaagtgggcatctggagcggccccgcccctcagtgcattatacctggtggaggtggaagtggaggtggtgga |
| | #10) | tctggtggaggtggatcaggaggtggtggatctggtggaggtggaagtatcttttgtccaagtcctccagttattcctaatgggaga |
| | D | cacacaggaaaacctctggaagtctttccctttgggaaaacagtaaattacacatgtgacccccacccagacagagggactagctt |
| | | tgacctcattggagagagcaccatccgctgcacaagtgaccctcaagggaatggggtttggagcagccctgcccctaggtgtgg |
| | | aattctgggtcactgtcaagccccagatcattttctgtttgccaagttgaaaacccaaaccaatgcatctgactttcccattgggacat |
| | | ctttaaagtatgaatgccgccctgagtactatgggaggccattctctatcacatgtctagataacctggtctggtcaagtcccaaaga |
| | | tgtctgtaaaaggaaatcatgtaaaactcctccagatccagtgaatggcatggtgcatgtgatcacagacatccaggttggatccag |
| | | aatcaactattcttgtactactgggcaccgcctcattggtcactcatctgctgaatgtatcctcagtggcaatgctgcccattggagca |
| | | ctaagccgccaatttgtcaaaggattccttgtgggctaccccccaccattgccaatggagatttcattagcaccaacagagagaattt |
| | | tcactatggatcagtggtgacctaccgctgcaatcctggaagcggagggagaaaggtgtttgagcttgtgggtgagccctccatat |
| | | actgcaccagcaatgatgatcaagtgggcatctggagcggcccagcccctcagtgcattatacctggtggaggtggatcaggag |
| | | gtggtggatctggtggaggtggaagtggaggtggtggatctggtggaggtggatcaatcttttgtccaagtcctccagttattccta |
| | | atgggagacacacaggaaaacctctggaagtctttccctttggaaaagcagtaaattacacatgtgacccccacccagacagagg |
| | | gactagctttgacctcattggagagagcaccatccgctgcacaagtgaccctcaagggaatggggtttggagcagccctgcccct |
| | | agatgtggaattctgggtcactgtcaagccccagatcattttctgtttgccaagttgaaaacccaaaccaatgcatctgactttcccatt |
| | | gggacatctttaaagtatgaatgccgccctgagtactatgggaggccattctctatcacatgtctagataacctggtctggtcaagtc |
| | | ccaaagatgtctgtaaaaggaaatcatgtaaaactcctccagatccagtgaatggcatggtgcatgtgatcacagacatccaggtt |
| | | ggatccagaatcaactattcttgtactactgggcaccgcctcattggtcactcatctgctgaatgtatcctctcaggcaatactgccca |
| | | ttggagcactaagccgccaatttgtcaaagaattccttgtgggctacccccaaccattgccaatggagatttcattagcaccaacag |
| | | agagaattttcactatggatcagtggtgacctaccgctgcaatcttggaagcagagggagaaaggtgtttgagcttgtgggtgagc |
| | | cctccatatactgcaccagcaatgatgatcaagtgggcatctggagcggccccgcccctcagtgcattatatag |
| 123 | CR1 | caatgcaatgccccagaatggcttccatttgccaggcctaccaacctaactgatgaatttgagtttcccattgggacatatctgaact |
| | SCR1-3- | atgaatgccgccctggttatagcggaagaccattttctatcatctgcctaaaaaactcagtctggactggtgctaaggacaggtgca |
| | linker- | gaaggaaatcatgtaggaatcctccagatcctgtgaatggcatggtgcatgtgatcaaaggcatccagtttggatcccaaattaaat |
| | 7-10- | attcttgtactaaaggataccgcctcattggttccagttctgccacatgcatcatctcaggtgatactgtcatttgggataatgaaacac |
| | linker | ctatttgtgacagaattccttgtgggctaccccccaccatcaccaatggagatttcattagcaccaacagagagaattttcactatgg |
| | 14-17 | atcagtggtgacctaccgctgcaatcctggaagcggagggagaaaggtgtttgagcttgtgggtgagccctccatatactgcacc |
| | (CR2CR1 | agcaatgatgatcaagtgggcatctggagcggccccgcccctcagtgcattatacctggtggaggtggatcaggaggtggtgga |
| | #11) | tctggtggaggtggaagtggaggtggtggatctggtggaggtggatcaggaggtggtggatctggtggaggtggaagtggagg |
| | A | tggtggatctggtggaggtggatcaggaggtggtggatctggtggaggtggaagtggaggtggtggatctatcttttgtccaagtc |
| | | ctccagttattcctaatgggagacacacaggaaaacctctggaagtctttccctttgggaaaacagtaaattacacatgtgaccccc |
| | | acccagacagagggactagctttgacctcattggagagagcaccatccgctgcacaagtgaccctcaagggaatggggtttgga |
| | | gcagccctgcccctaggtgtggaattctgggtcactgtcaagccccagatcattttctgtttgccaagttgaaaacccaaaccaatg |
| | | catctgactttcccattgggacatctttaaagtatgaatgccgccctgagtactatgggaggccattctctatcacatgtctagataac |
| | | ctggtctggtcaagtcccaaagatgtctgtaaaaggaaatcatgtaaaactcctccagatccagtgaatggcatggtgcatgtgatc |
| | | acagacatccaggttggatccagaatcaactattcttgtactactgggcaccgcctcattggtcactcatctgctgaatgtatcctctc |
| | | aggcaatgctgcccattggagcactaagccgccaatttgtcaaaggattccttgtgggctaccccccaccattgccaatggagattt |
| | | cattagcaccaacagagagaattttcactatggatcagtggtgacctaccgctgcaatcctggaagcggagggagaaaggtgttt |
| | | gagcttgtgggtgagccctccatatactgcaccagcaatgatgatcaagtgggcatctggagcggcccagcccctcagtgcattat |
| | | acctggtggaggtggaagtggaggtggtggatctggtggaggtggatcaggaggtggtggatctggtggaggtggaagtggag |
| | | gtggtggatctggtggaggtggatcaggaggtggtggatctggtggaggtggaagtggaggtggtggatctggtggaggtggat |
| | | caggaggtggtggatctatcttttgtccaagtcctccagttattcctaatgggagacacacaggaaaacctctggaagtctttcccttt |
| | | ggaaaagcagtaaattacacatgtgacccccacccagacagagggactagctttgacctcattggagagagcaccatccgctgc |
| | | acaagtgaccctcaagggaatggggtttggagcagccctgcccctagatgtggaattctgggtcactgtcaagccccagatcattt |
| | | tctgtttgccaagttgaaaacccaaaccaatgcatctgactttcccattgggacatctttaaagtatgaatgccgccctgagtactatg |
| | | ggaggccattctctatcacatgtctagataacctggtctggtcaagtcccaaagatgtctgtaaaaggaaatcatgtaaaactcctcc |
| | | agatccagtgaatggcatggtgcatgtgatcacagacatccaggttggatccagaatcaactattcttgtactactgggcaccgcct |
| | | cattggtcactcatctgctgaatgtatcctctcaggcaatactgcccattggagcactaagccgccaatttgtcaaagaattccttgtg |
| | | ggctacccccaaccattgccaatggagatttcattagcaccaacagagagaattttcactatggatcagtggtgacctaccgctgca |
| | | atcttggaagcagagggagaaaggtgtttgagcttgtgggtgagccctccatatactgcaccagcaatgatgatcaagtgggcatc |
| | | tggagcggccccgcccctcagtgcattatatag |
| 124 | CR1 | caatgcaatgccccagaatggcttccatttgccaggcctaccaacctaactgatgaatttgagtttcccattgggacatatctgaact |
| | SCR1-3- | atgaatgccgccctggttatagcggaagaccattttctatcatctgcctaaaaaactcagtctggactggtgctaaggacaggtgca |
| | linker- | gaaggaaatcatgtaggaatcctccagatcctgtgaatggcatggtgcatgtgatcaaaggcatccagtttggatcccaaattaaat |
| | 7-10- | attcttgtactaaaggataccgcctcattggttccagttctgccacatgcatcatctcaggtgatactgtcatttgggataatgaaacac |
| | linker | ctatttgtgacagaattccttgtgggctaccccccaccatcaccaatggagatttcattagcaccaacagagagaattttcactatgg |
| | 14-17 | atcagtggtgacctaccgctgcaatcctggaagcggagggagaaaggtgtttgagcttgtgggtgagccctccatatactgcacc |
| | (CR2CR1 | agcaatgatgatcaagtgggcatctggagcggccccgcccctcagtgcattatacctggtggaggtggatcaggaggtggtgga |
| | #11) | tctggtggaggtggaagtggaggtggtggatctggtggaggtggatcaatcttttgtccaagtcctccagttattcctaatgggaga |
| | B | cacacaggaaaacctctggaagtctttccctttgggaaaacagtaaattacacatgtgacccccacccagacagagggactagctt |
| | | tgacctcattggagagagcaccatccgctgcacaagtgaccctcaagggaatggggtttggagcagccctgcccctaggtgtgg |
| | | aattctgggtcactgtcaagccccagatcattttctgtttgccaagttgaaaacccaaaccaatgcatctgactttcccattgggacat |
| | | ctttaaagtatgaatgccgccctgagtactatgggaggccattctctatcacatgtctagataacctggtctggtcaagtcccaaaga |
| | | tgtctgtaaaaggaaatcatgtaaaactcctccagatccagtgaatggcatggtgcatgtgatcacagacatccaggttggatccag |
| | | aatcaactattcttgtactactgggcaccgcctcattggtcactcatctgctgaatgtatcctctcaggcaatgctgcccattggagca |
| | | ctaagccgccaatttgtcaaaggattccttgtgggctaccccccaccattgccaatggagatttcattagcaccaacagagagaattt |
| | | tcactatggatcagtggtgacctaccgctgcaatcctggaagcggagggagaaaggtgtttgagcttgtgggtgagccctccatat |
| | | actgcaccagcaatgatgatcaagtgggcatctggagcggcccagcccctcagtgcattatacctggtggaggtggaagtggag |
| | | gtggtggatctggtggaggtggatcaggaggtggtggatctggtggaggtggaagtatcttttgtccaagtcctccagttattccta |
| | | atgggagacacacaggaaaacctctggaagtctttccctttggaaaagcagtaaattacacatgtgacccccacccagacagagg |
| | | gactagctttgacctcattggagagagcaccatccgctgcacaagtgaccctcaagggaatggggtttggagcagccctgcccct |
| | | agatgtggaattctgggtcactgtcaagccccagatcattttctgtttgccaagttgaaaacccaaaccaatgcatctgactttcccatt |
| | | gggacatctttaaagtatgaatgccgccctgagtactatgggaggccattctctatcacatgtctagataacctggtctggtcaagtc |
| | | ccaaagatgtctgtaaaaggaaatcatgtaaaactcctccagatccagtgaatggcatggtgcatgtgatcacagacatccaggtt |
| | | ggatccagaatcaactattcttgtactactgggcaccgcctcattggtcactcatctgctgaatgtatcctctcaggcaatactgccca |
| | | ttggagcactaagccgccaatttgtcaaagaattccttgtgggctacccccaaccattgccaatggagatttcattagcaccaacag |
| | | agagaattttcactatggatcagtggtgacctaccgctgcaatcttggaagcagagggagaaaggtgtttgagcttgtgggtgagc |
| | | cctccatatactgcaccagcaatgatgatcaagtgggcatctggagcggccccgcccctcagtgcattatatag |
| 125 | CR1 | caatgcaatgccccagaatggcttccatttgccaggcctaccaacctaactgatgaatttgagtttcccattgggacatatctgaact |
| | SCR1-4, | atgaatgccgccctggttattccggaagaccgttttctatcatctgcctaaaaaactcagtctggactggtgctaaggacaggtgca |
| | 6-12, | gacgtaaatcatgtcgtaatcctccagatcctgtgaatggcatggtgcatgtgatcaaaggcatccagttcggatcccaaattaaata |
| | 14-17 | ttcttgtactaaaggataccgactcattggttcctcgtctgccacatgcatcatctcaggtgatactgtcatttgggataatgaaacacc |
| | (CR2CR1 | tatttgtgacagaattccttgtgggctaccccccaccatcaccaatggagatttcattagcaccaacagagagaattttcactatggat |
| | #12) | cagtggtgacctaccgctgcaatcctggaagcggagggagaaaggtgtttgagcttgtgggtgagccctccatatactgcaccag |
| | A | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccagggtatgtcagccacctccagatgt |
| | | cctgcatgctgagcgtacccaaagggacaaggacaacttttcacctgggcaggaagtgttctacagctgtgagcccggctacga |
| | | cctcagaggggctgcgtctatgcgctgcacaccccagggagactggagccctgcagcccccacatgtgaagtgatcttttgtcca |
| | | agtcctccagttattcctaatgggagacacacaggaaaacctctggaagtctttccctttgggaaaacagtaaattacacatgcgac |
| | | ccccacccagacagagggacgagcttcgacctcattggagagagcaccatccgctgcacaagtgaccctcaagggaatggggt |
| | | ttggagcagccctgcccctcgctgtggaattctgggtcactgtcaagccccagatcattttctgtttgccaagttgaaaacccaaacc |
| | | aatgcatctgactttcccattgggacatctttaaagtacgaatgccgtcctgagtactacgggaggccattctctatcacatgtctaga |
| | | taacctggtctggtcaagtcccaaagatgtctgtaaacgtaaatcatgtaaaactcctccagatccagtgaatggcatggtgcatgtg |
| | | atcacagacatccaggttggatccagaatcaactattcttgtactacagggcaccgactcattggtcactcatctgctgaatgtatcct |
| | | ctcgggcaatgctgcccattggagcacgaagccgccaatttgtcaacgaattccttgtgggctaccccccaccatcgccaatgga |
| | | gatttcattagcaccaacagagagaattttcactatggatcagtggtgacctaccgctgcaatcctggaagcggagggagaaaggt |
| | | gtttgagcttgtgggtgagccctccatatactgcaccagcaatgacgatcaagtgggcatctggagcggcccggcccctcagtgc |
| | | attatacctaacaaatgcacgcctccaaatgtggaaaatggaatattggtatctgacaacagaagcttattttccttaaatgaagttgtg |
| | | gagtttaggtgtcagcctggctttgtcatgaaaggaccccgccgtgtgaagtgccaggccctgaacaaatgggagccggagcta |
| | | ccaagctgctccagggtatgtcagccacctccagatgtcctgcatgctgagcgtacccaaagggacaaggacaacttttcacccg |
| | | ggcaggaagtgttctacagctgtgagcccggctatgacctcagaggggctgcgtctatgcgctgcacaccccagggagactgg |
| | | agccctgcagcccccacatgtgaagtgatcttttgtccaagtcctccagttattcctaatgggagacacacaggaaaacctctggaa |
| | | gtctttccctttggaaaagcagtaaattacacatgcgacccccacccagacagagggacgagcttcgacctcattggagagagca |
| | | ccatccgctgcacaagtgaccctcaagggaatggggtttggagcagccctgcccctcgctgtggaattctgggtcactgtcaagc |
| | | cccagatcattttctgtttgccaagttgaaaacccaaaccaatgcatctgactttcccattgggacatctttaaagtacgaatgccgtc |
| | | ctgagtactacgggaggccattctctatcacatgtctagataacctggtctggtcaagtcccaaagatgtctgtaaacgtaaatcatg |
| | | taaaactcctccagatccagtgaatggcatggtgcatgtgatcacagacatccaggttggatccagaatcaactattcttgtactaca |
| | | gggcaccgactcattggtcactcatctgctgaatgtatcctctcaggcaatactgcccattggagcacgaagccgccaatttgtcaa |
| | | cgaattccttgtgggctacccccaaccatcgccaatggagatttcattagcaccaacagagagaattttcactatggatcagtggtg |
| | | acctaccgctgcaatcttggaagcagagggagaaaggtgtttgagcttgtgggtgagccctccatatactgcaccagcaatgacg |
| | | atcaagtgggcatctggagcggccccgcccctcagtgcattatatag |
| 126 | CR1 | caatgcaatgccccagaatggcttccatttgccaggcctaccaacctaactgatgaatttgagtttcccattgggacatatctgaact |
| | SCR1-4, | atgaatgccgccctggttatagcggaagaccattttctatcatctgcctaaaaaactcagtctggactggtgctaaggacaggtgca |
| | 6-12, | gaaggaaatcatgtaggaatcctccagatcctgtgaatggcatggtgcatgtgatcaaaggcatccagtttggatcccaaattaaat |
| | 14-17 | attcttgtactaaaggataccgcctcattggttccagttctgccacatgcatcatctcaggtgatactgtcatttgggataatgaaacac |
| | (CR2CR1 | ctatttgtgacagaattccttgtgggctaccccccaccatcaccaatggagatttcattagcaccaacagagagaattttcactatgg |
| | #12) | atcagtggtgacctaccgctgcaatcctggaagcggagggagaaaggtgtttgagcttgtgggtgagccctccatatactgcacc |
| | B | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccagggtatgtcagccacctccagat |
| | | gtcctgcatgctgagcgcacccaaagggacaaggacaacttttcacctgggcaggaagtgttctacagctgtgagcctggctatg |
| | | acctcagaggggctgcatctatgcgctgcacaccccagggagactggagccctgcagcccccacatgtgaagtgatcttttgtcc |
| | | aagtcctccagttattcctaatgggagacacacaggaaaacctctggaagtctttccctttgggaaaacagtaaattacacatgtgac |
| | | ccccacccagacagagggactagctttgacctcattggagagagcaccatccgctgcacaagtgaccctcaagggaatggggtt |
| | | tggagcagccctgcccctaggtgtggaattctgggtcactgtcaagccccagatcattttctgtttgccaagttgaaaacccaaacc |
| | | aatgcatctgactttcccattgggacatctttaaagtatgaatgccgccctgagtactatgggaggccattctctatcacatgtctagat |
| | | aacctggtctggtcaagtcccaaagatgtctgtaaaaggaaatcatgtaaaactcctccagatccagtgaatggcatggtgcatgtg |
| | | atcacagacatccaggttggatccagaatcaactattcttgtactactgggcaccgcctcattggtcactcatctgctgaatgtatcct |
| | | cagtggcaatgctgcccattggagcactaagccgccaatttgtcaaaggattccttgtgggctaccccccaccattgccaatggag |
| | | atttcattagcaccaacagagagaattttcactatggatcagtggtgacctaccgctgcaatcctggaagcggagggagaaaggtg |
| | | tttgagcttgtgggtgagccctccatatactgcaccagcaatgatgatcaagtgggcatctggagcggcccagcccctcagtgcat |
| | | tatacctaacaaatgcactcctccaaatgtggaaaatggaatattggtatctgacaacagaagcttattttccttaaatgaagttgtgga |
| | | gtttaggtgtcagcctggctttgtcatgaaaggaccccgcagggtgaagtgccaggccctgaacaaatgggagccagagctacc |
| | | aagctgctccagggtatgtcagccacctccagatgtcctgcatgctgagcgcacccaaagggacaaggacaacttttcacctggg |
| | | caggaagtgttctacagctgtgagcctggctatgacctcagaggggctgcatctatgcgctgcacaccccagggagactggagc |
| | | cctgcagcccccacatgtgaagtgatcttttgtccaagtcctccagttattcctaatgggagacacacaggaaaacctctggaagtc |
| | | tttccctttggaaaagcagtaaattacacatgtgacccccacccagacagagggactagctttgacctcattggagagagcaccat |
| | | ccgctgcacaagtgaccctcaagggaatggggtttggagcagccctgcccctaggtgtggaattctgggtcactgtcaagcccca |
| | | gatcattttctgtttgccaagttgaaaacccaaaccaatgcatctgactttcccattgggacatctttaaagtatgaatgccgccctgag |
| | | tactatgggaggccattctctatcacatgtctagataacctggtctggtcaagtcccaaagatgtctgtaaaagaaaatcatgtaaaa |
| | | ctcctccagatccagtgaatggcatggtgcatgtgatcacagacatccaggttggatccagaatcaactattcttgtactactgggca |
| | | ccgcctcattggtcactcatctgctgaatgtatcctctcaggcaatactgcccattggagcactaagccgccaatttgtcaaaggatt |
| | | ccttgtgggctacccccaaccattgccaatggagatttcattagcaccaacagagagaattttcactatggatcagtggtgacctac |
| | | cgctgcaatcttggaagcagagggagaaaggtgtttgagcttgtgggtgagccctccatatactgcaccagcaatgatgatcaagt |
| | | gggcatctggagcggccccgcccctcagtgcattatatag |
| 127 | CR1 | caatgcaatgccccagaatggcttccatttgccaggcctaccaacctaactgatgaatttgagtttcccattgggacatatctgaact |
| | SCR1-10 | atgaatgccgccctggttattccggaagaccgttttctatcatctgcctaaaaaactcagtctggactggtgctaaggacaggtgca |
| | (CR2CR1 | gacgtaaatcatgtcgtaatcctccagatcctgtgaatggcatggtgcatgtgatcaaaggcatccagttcggatcccaaattaaata |
| | #1) A | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccagggtatgtcagccacctccagatgt |
| | | cctgcatgctgagcgtacccaaagggacaaggacaacttttcacctgggcaggaagtgttctacagctgtgagcccggctacga |
| | | cctcagaggggctgcgtctatgcgctgcacaccccagggagactggagccctgcagcccccacatgtgaagtgaaatcctgtga |
| | | tgacttcatgggccaacttcttaatggccgtgtgctatttccagtaaatctccagcttggagcaaaagtggattttgtttgtgatgaagg |
| | | atttcaattaaaaggcagctctgctagttactgtgtcttggctggaatggaaagcctttggaatagcagtgttccagtgtgtgaacaaa |
| | | tcttttgtccaagtcctccagttattcctaatgggagacacacaggaaaacctctggaagtctttccctttgggaaaacagtaaattac |
| | | acatgcgacccccacccagacagagggacgagcttcgacctcattggagagagcaccatccgctgcacaagtgaccctcaagg |
| | | gaatggggtttggagcagccctgcccctcgctgtggaattctgggtcactgtcaagccccagatcattttctgtttgccaagttgaaa |
| | | acccaaaccaatgcatctgactttcccattgggacatctttaaagtacgaatgccgtcctgagtactacgggaggccattctctatca |
| | | catgtctagataacctggtctggtcaagtcccaaagatgtctgtaaacgtaaatcatgtaaaactcctccagatccagtgaatggcat |
| | | ggtgcatgtgatcacagacatccaggttggatccagaatcaactattcttgtactacagggcaccgactcattggtcactcatctgct |
| | | gaatgtatcctctcgggcaatgctgcccattggagcacgaagccgccaatttgtcaacgaattccttgtgggctaccccccaccatc |
| | | gccaatggagatttcattagcaccaacagagagaattttcactatggatcagtggtgacctaccgctgcaatcctggaagcggagg |
| | | gagaaaggtgtttgagcttgtgggtgagccctccatatactgcaccagcaatgacgatcaagtgggcatctggagcggcccggc |
| | | ccctcagtgcattatacctaacaaatag |
| 128 | CR1 | caatgcaatgccccagaatggcttccatttgccaggcctaccaacctaactgatgaatttgagtttcccattgggacatatctgaact |
| | SCR1-10 | atgaatgccgccctggttatagcggaagaccattttctatcatctgcctaaaaaactcagtctggactggtgctaaggacaggtgca |
| | (CR2CR1 | gaaggaaatcatgtagaaatcctccagatcctgtgaatggcatggtgcatgtgatcaaaggcatccagtttggatcccaaattaaat |
| | #1) B | attcttgtactaaaggataccgcctcattggttcctcatctgccacatgcatcatctcaggtgatactgtcatttgggataatgaaacac |
| | | ctatttgtgacagaattccttgtgggctaccccccaccatcaccaatggagatttcattagcaccaacagagagaattttcactatgg |
| | | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccagggtatgtcagccacctccagat |
| | | gtcctgcatgctgagcgcacccaaagggacaaggacaacttttcacctgggcaggaagtgttctacagctgtgagcctggctatg |
| | | acctcagaggggctgcatctatgcgctgcacaccccagggagactggagccctgcagcccccacatgtgaagtgaaatcctgtg |
| | | atgacttcatgggccaacttcttaatggcagagtgctatttccagtaaatctccagcttggagcaaaagtggattttgtttgtgatgaag |
| | | gatttcaattaaaaggcagctctgctagttactgtgtcttggctggaatggaaagcctttggaatagcagtgttccagtgtgtgaacaa |
| | | atcttttgtccaagtcctccagttattcctaatgggagacacacaggaaaacctctggaagtctttccctttgggaaaacagtaaatta |
| | | cacatgtgacccccacccagacagagggactagctttgacctcattggagagagcaccatccgctgcacaagtgaccctcaagg |
| | | gaatggggtttggagcagccctgcccctaggtgtggaattctgggtcactgtcaagccccagatcattttctgtttgccaagttgaaa |
| | | acccaaaccaatgcatctgactttcccattgggacatctttaaagtatgaatgccgccctgagtactatgggaggccattctctatca |
| | | catgtctagataacctggtctggtcaagtcccaaagatgtctgtaaaagaaaatcatgtaaaactcctccagatccagtgaatggcat |
| | | ggtgcatgtgatcacagacatccaggttggatccagaatcaactattcttgtacaactgggcaccgcctcattggtcactcatctgct |
| | | gaatgtatcctctcaggcaatgctgcccattggagcactaagccgccaatttgtcaaaggattccttgtgggctaccccccaccatt |
| | | gccaatggagatttcattagcaccaacagagagaattttcactatggatcagtggtgacctaccgctgcaatcctggaagcggagg |
| | | gagaaaggtgtttgagcttgtgggtgagccctccatatactgcaccagcaatgatgatcaagtgggcatctggagcggcccagcc |
| | | cctcagtgcattatacctaacaaatag |
| 129 | CR1 | caatgcaatgccccagaatggcttccatttgccaggcctaccaacctaactgatgaatttgagtttcccattgggacatatctgaact |
| | SCR1-10 | atgaatgccgccctggttattccggaagaccgttttctatcatctgcctaaaaaactcagtctggactggtgctaaggacaggtgca |
| | (CR2CR1 | gacgtaaatcatgtcgtaatcctccagatcctgtgaatggcatggtgcatgtgatcaaaggcatccagttcggatcccaaattaaata |
| | #1) C | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccagggtatgtcagccacctccagatgt |
| | | cctgcatgctgagcgtacccaaagggacaaggacaacttttcacctgggcaggaagtgttctacagctgtgagcccggctacga |
| | | cctcagaggggctgcgtctatgcgctgcacaccccagggagactggagccctgcagcccccacatgtgaagtgaaatcctgtga |
| | | tgacttcatgggccaacttcttaatggccgtgtgctatttccagtaaatctccagcttggagcaaaagtggattttgtttgtgatgaagg |
| | | atttcaattaaaaggcagctctgctagttactgtgtcttggctggaatggaaagcctttggaatagcagtgttccagtgtgtgaacaaa |
| | | tcttttgtccaagtcctccagttattcctaatgggagacacacaggaaaacctctggaagtctttccctttgggaaaacagtaaattac |
| | | acatgcgacccccacccagacagagggacgagcttcgacctcattggagagagcaccatccgctgcacaagtgaccctcaagg |
| | | gaatggggtttggagcagccctgcccctcgctgtggaattctgggtcactgtcaagccccagatcattttctgtttgccaagttgaaa |
| | | acccaaaccaatgcatctgactttcccattgggacatctttaaagtacgaatgccgtcctgagtactacgggaggccattctctatca |
| | | catgtctagataacctggtctggtcaagtcccaaagatgtctgtaaacgtaaatcatgtaaaactcctccagatccagtgaatggcat |
| | | ggtgcatgtgatcacagacatccaggttggatccagaatcaactattcttgtactacagggcaccgactcattggtcactcatctgct |
| | | gaatgtatcctctcgggcaatgctgcccattggagcacgaagccgccaatttgtcaacgaattccttgtgggctaccccccaccatc |
| | | gccaatggagatttcattagcaccaacagagagaattttcactatggatcagtggtgacctaccgctgcaatcctggaagcggagg |
| | | gagaaaggtgtttgagcttgtgggtgagccctccatatactgcaccagcaatgacgatcaagtgggcatctggagcggcccggc |
| | | ccctcagtgcattatatag |
| 130 | CR1 | caatgcaatgccccagaatggcttccatttgccaggcctaccaacctaactgatgaatttgagtttcccattgggacatatctgaact |
| | SCR1-10 | atgaatgccgccctggttatagcggaagaccattttctatcatctgcctaaaaaactcagtctggactggtgctaaggacaggtgca |
| | (CR2CR1 | gaaggaaatcatgtagaaatcctccagatcctgtgaatggcatggtgcatgtgatcaaaggcatccagtttggatcccaaattaaat |
| | #2) | attcttgtactaaaggataccgcctcattggttcctcatctgccacatgcatcatctcaggtgatactgtcatttgggataatgaaacac |
| | | ctatttgtgacagaattccttgtgggctaccccccaccatcaccaatggagatttcattagcaccaacagagagaattttcactatgg |
| | | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagggtgaagtgccaggccctgaacaaatgggagccagagctaccaagctgctccagggtatgtcagccacctccagat |
| | | gtcctgcatgctgagcgcacccaaagggacaaggacaacttttcacctgggcaggaagtgttctacagctgtgagcctggctatg |
| | | acctcagaggggctgcatctatgcgctgcacaccccagggagactggagccctgcagcccccacatgtgaagtgaaatcctgtg |
| | | atgacttcatgggccaacttcttaatggcagagtgctatttccagtaaatctccagcttggagcaaaagtggattttgtttgtgatgaag |
| | | gatttcaattaaaaggcagctctgctagttactgtgtcttggctggaatggaaagcctttggaatagcagtgttccagtgtgtgaacaa |
| | | atcttttgtccaagtcctccagttattcctaatgggagacacacaggaaaacctctggaagtctttccctttgggaaaacagtaaatta |
| | | cacatgtgacccccacccagacagagggactagctttgacctcattggagagagcaccatccgctgcacaagtgaccctcaagg |
| | | gaatggggtttggagcagccctgcccctaggtgtggaattctgggtcactgtcaagccccagatcattttctgtttgccaagttgaaa |
| | | acccaaaccaatgcatctgactttcccattgggacatctttaaagtatgaatgccgccctgagtactatgggaggccattctctatca |
| | | catgtctagataacctggtctggtcaagtcccaaagatgtctgtaaaagaaaatcatgtaaaactcctccagatccagtgaatggcat |
| | | ggtgcatgtgatcacagacatccaggttggatccagaatcaactattcttgtacaactgggcaccgcctcattggtcactcatctgct |
| | | gaatgtatcctcagtggcaatgctgcccattggagcactaagccgccaatttgtcaaaggattccttgtgggctaccccccaccatt |
| | | gccaatggagatttcattagcaccaacagagagaattttcactatggatcagtggtgacctaccgctgcaatcctggaagcggagg |
| | | gagaaaggtgtttgagcttgtgggtgagccctccatatactgcaccagcaatgatgatcaagtgggcatctggagcggcccagcc |
| | | cctcagtgcattatacctaacaaatag |
| 131 | CR1 | caatgcaatgccccagaatggcttccatttgccaggcctaccaacctaactgatgaatttgagtttcccattgggacatatctgaact |
| | SCR1-3, | atgaatgccgccctggttattccggaagaccgttttctatcatctgcctaaaaaactcagtctggactggtgctaaggacaggtgca |
| | 8-10, | gacgtaaatcatgtcgtaatcctccagatcctgtgaatggcatggtgcatgtgatcaaaggcatccagttcggatcccaaattaaata |
| | 15-17 | ttcttgtactaaaggataccgactcattggttcctcgtctgccacatgcatcatctcaggtgatactgtcatttgggataatgaaacacc |
| | (CR2CR1 | tatttgtgacagaattccttgtgggctaccccccaccatcaccaatggagatttcattagcaccaacagagagaattttcactatggat |
| | #3) A | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcataattggtcactgtcaagccccagatcattttctgtttgc |
| | | caagttgaaaacccaaaccaatgcatctgactttcccattgggacatctttaaagtacgaatgccgtcctgagtactacgggaggcc |
| | | attctctatcacatgtctagataacctggtctggtcaagtcccaaagatgtctgtaaacgtaaatcatgtaaaactcctccagatccag |
| | | tgaatggcatggtgcatgtgatcacagacatccaggttggatccagaatcaactattcttgtactacagggcaccgactcattggtc |
| | | actcatctgctgaatgtatcctctcgggcaatgctgcccattggagcacgaagccgccaatttgtcaacgaattccttgtgggctacc |
| | | ccccaccatcgccaatggagatttcattagcaccaacagagagaattttcactatggatcagtggtgacctaccgctgcaatcctgg |
| | | aagcggagggagaaaggtgtttgagcttgtgggtgagccctccatatactgcaccagcaatgacgatcaagtgggcatctggag |
| | | cggcccggcccctcagtgcattatacctaacaaaggtcactgtcaagccccagatcattttctgtttgccaagttgaaaacccaaac |
| | | caatgcatctgactttcccattgggacatctttaaagtacgaatgccgtcctgagtactacgggaggccattctctatcacatgtctag |
| | | ataacctggtctggtcaagtcccaaagatgtctgtaaacgtaaatcatgtaaaactcctccagatccagtgaatggcatggtgcatgt |
| | | gatcacagacatccaggttggatccagaatcaactattcttgtactacagggcaccgactcattggtcactcatctgctgaatgtatc |
| | | ctctcaggcaatactgcccattggagcacgaagccgccaatttgtcaacgaattccttgtgggctacccccaaccatcgccaatgg |
| | | agatttcattagcaccaacagagagaattttcactatggatcagtggtgacctaccgctgcaatcttggaagcagagggagaaagg |
| | | tgtttgagcttgtgggtgagccctccatatactgcaccagcaatgacgatcaagtgggcatctggagcggccccgcccctcagtg |
| | | cattatatag |
| 132 | CR1 | caatgcaatgccccagaatggcttccatttgccaggcctaccaacctaactgatgaatttgagtttcccattgggacatatctgaact |
| | SCR1-3, | atgaatgccgccctggttatagcggaagaccattttctatcatctgcctaaaaaactcagtctggactggtgctaaggacaggtgca |
| | 8-10, | gaaggaaatcatgtagaaatcctccagatcctgtgaatggcatggtgcatgtgatcaaaggcatccagtttggatcccaaattaaat |
| | 15-17 | attcttgtactaaaggataccgcctcattggttccagttctgccacatgcatcatctcaggtgatactgtcatttgggataatgaaacac |
| | (CR2CR1 | ctatttgtgacagaattccttgtgggctaccccccaccatcaccaatggagatttcattagcaccaacagagagaattttcactatgg |
| | #3) B | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcataattggtcactgtcaagccccagatcattttctgttt |
| | | gccaagttgaaaacccaaaccaatgcatctgactttcccattgggacatctttaaagtatgaatgccgccctgagtactatgggagg |
| | | ccattctctatcacatgtctagataacctggtctggtcaagtcccaaagatgtctgtaaaaggaaatcatgtaaaactcctccagatcc |
| | | agtgaatggcatggtgcatgtgatcacagacatccaggttggatccagaatcaactattcttgtacaactgggcaccgcctcattgg |
| | | tcactcatctgctgaatgtatcctcagtggcaatgctgcccattggagcactaagccgccaatttgtcaaagaattccttgtgggcta |
| | | ccccccaccattgccaatggagatttcattagcaccaacagagagaattttcactatggatcagtggtgacctaccgctgcaatcct |
| | | ggaagcggagggagaaaggtgtttgagcttgtgggtgagccctccatatactgcaccagcaatgatgatcaagtgggcatctgg |
| | | agcggcccagcccctcagtgcattatacctaacaaaggtcactgtcaagccccagatcattttctgtttgccaagttgaaaacccaa |
| | | accaatgcatctgactttcccattgggacatctttaaagtatgaatgccgccctgagtactatgggaggccattctctatcacatgtct |
| | | agataacctggtctggtcaagtcccaaagatgtctgtaaaaggaaatcatgtaaaactcctccagatccagtgaatggcatggtgc |
| | | atgtgatcacagacatccaggttggatccagaatcaactattcttgtacaactgggcaccgcctcattggtcactcatctgctgaatgt |
| | | atcctctcaggcaatactgcccattggagcactaagccgccaatttgtcaaagaattccttgtgggctacccccaaccattgccaat |
| | | ggagatttcattagcaccaacagagagaattttcactatggatcagtggtgacctaccgctgcaatcttggaagcagagggagaaa |
| | | ggtgtttgagcttgtgggtgagccctccatatactgcaccagcaatgatgatcaagtgggcatctggagcggccccgcccctcagt |
| | | gcattatatag |
| 133 | CR1 | caatgcaatgccccagaatggcttccatttgccaggcctaccaacctaactgatgaatttgagtttcccattgggacatatctgaact |
| | SCR1-14 | atgaatgccgccctggttattccggaagaccgttttctatcatctgcctaaaaaactcagtctggactggtgctaaggacaggtgca |
| | (CR2CR1 | gacgtaaatcatgtcgtaatcctccagatcctgtgaatggcatggtgcatgtgatcaaaggcatccagttcggatcccaaattaaata |
| | #5) A | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccagggtatgtcagccacctccagatgt |
| | | cctgcatgctgagcgtacccaaagggacaaggacaacttttcacctgggcaggaagtgttctacagctgtgagcccggctacga |
| | | cctcagaggggctgcgtctatgcgctgcacaccccagggagactggagccctgcagcccccacatgtgaagtgaaatcctgtga |
| | | tgacttcatgggccaacttcttaatggccgtgtgctatttccagtaaatctccagcttggagcaaaagtggattttgtttgtgatgaagg |
| | | atttcaattaaaaggcagctctgctagttactgtgtcttggctggaatggaaagcctttggaatagcagtgttccagtgtgtgaacaaa |
| | | tcttttgtccaagtcctccagttattcctaatgggagacacacaggaaaacctctggaagtctttccctttgggaaaacagtaaattac |
| | | acatgcgacccccacccagacagagggacgagcttcgacctcattggagagagcaccatccgctgcacaagtgaccctcaagg |
| | | gaatggggtttggagcagccctgcccctcgctgtggaattctgggtcactgtcaagccccagatcattttctgtttgccaagttgaaa |
| | | acccaaaccaatgcatctgactttcccattgggacatctttaaagtacgaatgccgtcctgagtactacgggaggccattctctatca |
| | | catgtctagataacctggtctggtcaagtcccaaagatgtctgtaaacgtaaatcatgtaaaactcctccagatccagtgaatggcat |
| | | ggtgcatgtgatcacagacatccaggttggatccagaatcaactattcttgtactacagggcaccgactcattggtcactcatctgct |
| | | gaatgtatcctctcgggcaatgctgcccattggagcacgaagccgccaatttgtcaacgaattccttgtgggctaccccccaccatc |
| | | gccaatggagatttcattagcaccaacagagagaattttcactatggatcagtggtgacctaccgctgcaatcctggaagcggagg |
| | | gagaaaggtgtttgagcttgtgggtgagccctccatatactgcaccagcaatgacgatcaagtgggcatctggagcggcccggc |
| | | ccctcagtgcattatacctaacaaatgcacgcctccaaatgtggaaaatggaatattggtatctgacaacagaagcttattttccttaa |
| | | atgaagttgtggagtttaggtgtcagcctggctttgtcatgaaaggaccccgccgtgtgaagtgccaggccctgaacaaatggga |
| | | gccggagctaccaagctgctccagggtatgtcagccacctccagatgtcctgcatgctgagcgtacccaaagggacaaggaca |
| | | acttttcacccgggcaggaagtgttctacagctgtgagcccggctatgacctcagaggggctgcgtctatgcgctgcacacccca |
| | | gggagactggagccctgcagcccccacatgtgaagtgaaatcctgtgatgacttcatgggccaacttcttaatggccgtgtgctatt |
| | | tccagtaaatctccagcttggagcaaaagtggattttgtttgtgatgaaggatttcaattaaaaggcagctctgctagttattgtgtcttg |
| | | gctggaatggaaagcctttggaatagcagtgttccagtgtgtgaacaaatcttttgtccaagtcctccagttattcctaatgggagac |
| | | acacaggaaaacctctggaagtctttccctttggaaaagcagtaaattacacatgcgacccccacccagacagagggacgagctt |
| | | cgacctcattggagagagcaccatccgctgcacaagtgaccctcaagggaatggggtttggagcagccctgcccctcgctgtgg |
| | | aatttag |
| 134 | CR1 | caatgcaatgccccagaatggcttccatttgccaggcctaccaacctaactgatgaatttgagtttcccattgggacatatctgaact |
| | SCR1-14 | atgaatgccgccctggttatagcggaagaccattttctatcatctgcctaaaaaactcagtctggactggtgctaaggacaggtgca |
| | (CR2CR1 | gaagaaaatcatgtaggaatcctccagatcctgtgaatggcatggtgcatgtgatcaaaggcatccagtttggatcccaaattaaat |
| | #5) B | attcttgtactaaaggataccgcctcattggttcctcatctgccacatgcatcatctcaggtgatactgtcatttgggataatgaaacac |
| | | ctatttgtgacagaattccttgtgggctaccccccaccatcaccaatggagatttcattagcaccaacagagagaattttcactatgg |
| | | atcagtggtgacctaccgctgcaatcctggaagcggagggagaaaggtgtttgagcttgtgggtgagccctccatatactgcacc |
| | | agcaatgatgatcaagtgggcatctggagcggccccgcccctcagtgcattatacctaacaaatgcactcctccaaatgtggaaa |
| | | atggaatattggtatctgacaacagaagcttattttccttaaatgaagttgtggagtttaggtgtcagcctggctttgtcatgaaaggac |
| | | cccgcagagtgaagtgccaggccctgaacaaatgggagccagagctaccaagctgctccagggtatgtcagccacctccagat |
| | | gtcctgcatgctgagcgcacccaaagggacaaggacaacttttcacctgggcaggaagtgttctacagctgtgagcctggctatg |
| | | acctcagaggggctgcatctatgcgctgcacaccccagggagactggagccctgcagcccccacatgtgaagtgaaatcctgtg |
| | | atgacttcatgggccaacttcttaatggcagggtgctatttccagtaaatctccagcttggagcaaaagtggattttgtttgtgatgaa |
| | | ggatttcaattaaaaggcagctctgctagttactgtgtcttggctggaatggaaagcctttggaatagcagtgttccagtgtgtgaac |
| | | aaatcttttgtccaagtcctccagttattcctaatgggagacacacaggaaaacctctggaagtctttccctttgggaaaacagtaaat |
| | | tacacatgtgacccccacccagacagagggactagctttgacctcattggagagagcaccatccgctgcacaagtgaccctcaa |
| | | gggaatggggtttggagcagccctgcccctagatgtggaattctgggtcactgtcaagccccagatcattttctgtttgccaagttga |
| | | aaacccaaaccaatgcatctgactttcccattgggacatctttaaagtatgaatgccgccctgagtactatgggaggccattctctat |
| | | cacatgtctagataacctggtctggtcaagtcccaaagatgtctgtaaaaggaaatcatgtaaaactcctccagatccagtgaatgg |
| | | catggtgcatgtgatcacagacatccaggttggatccagaatcaactattcttgtacaactgggcaccgcctcattggtcactcatct |
| | | gctgaatgtatcctctcaggcaatgctgcccattggagcactaagccgccaatttgtcaaagaattccttgtgggctaccccccacc |
| | | attgccaatggagatttcattagcaccaacagagagaattttcactatggatcagtggtgacctaccgctgcaatcctggaagcgga |
| | | gggagaaaggtgtttgagcttgtgggtgagccctccatatactgcaccagcaatgatgatcaagtgggcatctggagcggcccag |
| | | cccctcagtgcattatacctaacaaatgcactcctccaaatgtggaaaatggaatattggtatctgacaacagaagcttattttccttaa |
| | | atgaagttgtggagtttaggtgtcagcctggctttgtcatgaaaggaccccgcagggtgaagtgccaggccctgaacaaatggga |
| | | gccagagctaccaagctgctccagggtatgtcagccacctccagatgtcctgcatgctgagcgcacccaaagggacaaggaca |
| | | acttttcacctgggcaggaagtgttctacagctgtgagcctggctatgacctcagaggggctgcatctatgcgctgcacaccccag |
| | | ggagactggagccctgcagcccccacatgtgaagtgaaatcctgtgatgacttcatgggccaacttcttaatggcagagtgctattt |
| | | ccagtaaatctccagcttggagcaaaagtggattttgtttgtgatgaaggatttcaattaaaaggcagctctgctagttattgtgtcttg |
| | | gctggaatggaaagcctttggaatagcagtgttccagtgtgtgaacaaatcttttgtccaagtcctccagttattcctaatgggagac |
| | | acacaggaaaacctctggaagtctttccctttggaaaagcagtaaattacacatgtgacccccacccagacagagggactagcttt |
| | | gacctcattggagagagcaccatccgctgcacaagtgaccctcaagggaatggggtttggagcagccctgcccctaggtgtgga |
| | | atttag |
| 135 | CR1 | caatgcaatgccccagaatggcttccatttgccaggcctaccaacctaactgatgaatttgagtttcccattgggacatatctgaact |
| | SCR1-17 | atgaatgccgccctggttattccggaagaccgttttctatcatctgcctaaaaaactcagtctggactggtgctaaggacaggtgca |
| | (CR2CR1 | gacgtaaatcatgtcgtaatcctccagatcctgtgaatggcatggtgcatgtgatcaaaggcatccagttcggatcccaaattaaata |
| | #13) | ttcttgtactaaaggataccgactcattggttcctcgtctgccacatgcatcatctcaggtgatactgtcatttgggataatgaaacacc |
| | | tatttgtgacagaattccttgtgggctaccccccaccatcaccaatggagatttcattagcaccaacagagagaattttcactatggat |
| | | cagtggtgacctaccgctgcaatcctggaagcggagggagaaaggtgtttgagcttgtgggtgagccctccatatactgcaccag |
| | | caatgacgatcaagtgggcatctggagcggccccgcccctcagtgcattatacctaacaaatgcacgcctccaaatgtggaaaat |
| | | ggaatattggtatctgacaacagaagcttattttccttaaatgaagttgtggagtttaggtgtcagcctggctttgtcatgaaaggacc |
| | | ccgccgtgtgaagtgccaggccctgaacaaatgggagccggagctaccaagctgctccagggtatgtcagccacctccagatgt |
| | | cctgcatgctgagcgtacccaaagggacaaggacaacttttcacctgggcaggaagtgttctacagctgtgagcccggctacga |
| | | cctcagaggggctgcgtctatgcgctgcacaccccagggagactggagccctgcagcccccacatgtgaagtgaaatcctgtga |
| | | tgacttcatgggccaacttcttaatggccgtgtgctatttccagtaaatctccagcttggagcaaaagtggattttgtttgtgatgaagg |
| | | atttcaattaaaaggcagctctgctagttactgtgtcttggctggaatggaaagcctttggaatagcagtgttccagtgtgtgaacaaa |
| | | tcttttgtccaagtcctccagttattcctaatgggagacacacaggaaaacctctggaagtctttccctttgggaaaacagtaaattac |
| | | acatgcgacccccacccagacagagggacgagcttcgacctcattggagagagcaccatccgctgcacaagtgaccctcaagg |
| | | gaatggggtttggagcagccctgcccctcgctgtggaattctgggtcactgtcaagccccagatcattttctgtttgccaagttgaaa |
| | | acccaaaccaatgcatctgactttcccattgggacatctttaaagtacgaatgccgtcctgagtactacgggaggccattctctatca |
| | | catgtctagataacctggtctggtcaagtcccaaagatgtctgtaaacgtaaatcatgtaaaactcctccagatccagtgaatggcat |
| | | ggtgcatgtgatcacagacatccaggttggatccagaatcaactattcttgtactacagggcaccgactcattggtcactcatctgct |
| | | gaatgtatcctctcgggcaatgctgcccattggagcacgaagccgccaatttgtcaacgaattccttgtgggctaccccccaccatc |
| | | gccaatggagatttcattagcaccaacagagagaattttcactatggatcagtggtgacctaccgctgcaatcctggaagcggagg |
| | | gagaaaggtgtttgagcttgtgggtgagccctccatatactgcaccagcaatgacgatcaagtgggcatctggagcggcccggc |
| | | ccctcagtgcattatacctaacaaatgcacgcctccaaatgtggaaaatggaatattggtatctgacaacagaagcttattttccttaa |
| | | atgaagttgtggagtttaggtgtcagcctggctttgtcatgaaaggaccccgccgtgtgaagtgccaggccctgaacaaatggga |
| | | gccggagctaccaagctgctccagggtatgtcagccacctccagatgtcctgcatgctgagcgtacccaaagggacaaggaca |
| | | acttttcacccgggcaggaagtgttctacagctgtgagcccggctatgacctcagaggggctgcgtctatgcgctgcacacccca |
| | | gggagactggagccctgcagcccccacatgtgaagtgaaatcctgtgatgacttcatgggccaacttcttaatggccgtgtgctatt |
| | | tccagtaaatctccagcttggagcaaaagtggattttgtttgtgatgaaggatttcaattaaaaggcagctctgctagttattgtgtcttg |
| | | gctggaatggaaagcctttggaatagcagtgttccagtgtgtgaacaaatcttttgtccaagtcctccagttattcctaatgggagac |
| | | acacaggaaaacctctggaagtctttccctttggaaaagcagtaaattacacatgcgacccccacccagacagagggacgagctt |
| | | cgacctcattggagagagcaccatccgctgcacaagtgaccctcaagggaatggggtttggagcagccctgcccctcgctgtgg |
| | | aattctgggtcactgtcaagccccagatcattttctgtttgccaagttgaaaacccaaaccaatgcatctgactttcccattgggacat |
| | | ctttaaagtacgaatgccgtcctgagtactacgggaggccattctctatcacatgtctagataacctggtctggtcaagtcccaaaga |
| | | tgtctgtaaacgtaaatcatgtaaaactcctccagatccagtgaatggcatggtgcatgtgatcacagacatccaggttggatccag |
| | | aatcaactattcttgtactacagggcaccgactcattggtcactcatctgctgaatgtatcctctcaggcaatactgcccattggagca |
| | | cgaagccgccaatttgtcaacgaattccttgtgggctacccccaaccatcgccaatggagatttcattagcaccaacagagagaat |
| | | tttcactatggatcagtggtgacctaccgctgcaatcttggaagcagagggagaaaggtgtttgagcttgtgggtgagccctccata |
| | | tactgcaccagcaatgacgatcaagtgggcatctggagcggccccgcccctcagtgcattatatag |
| 136 | VGFR1 | DTGRPFVEMYSEIPEIIHMTEGRELVIPCRVTSPNITVTLKKFPLDTLIPDGKRIIWDSR |
| | 130-232 | KGFIISNATYKEIGLLTCEATVNGHLYKTNYLTHRQTNTIIDV |
| 137 | VGFR2 | VLSPSHGIELSVGEKLVLNCTARTELNVGIDENWEYPSSKHQHKKLVNRDLKTQSG |
| | 227-327 | SEMKKFLSTLTIDGVTRSDQGLYTCAASSGLMTKKNSTFVRVHEK |
| | aa | |
| 138 | IgG1 | DKTHTCPPCPAPELLGGPSVFLFPPKPKDTLMISRTPEVTCVVVDVSHEDPEVKFNW |
| | Fc | YVDGVEVHNAKTKPREEQYNSTYRVVSVLTVLHQDWLNGKEYKCKVSNKALPAP |
| | portion | IEKTISKAKGQPREPQVYTLPPSRDELTKNQVSLTCLVKGFYPSDIAVEWESNGQPE |
| | #1 | NNYKTTPPVLDSDGSFFLYSKLTVDKSRWQQGNVFSCSVMHEALHNHYTQKSLSL |
| | | SPGK |
| 139 | Glycine | GGGGGG |
| | linker | |
| 140 | Full | DTGRPFVEMYSEIPEIIHMTEGRELVIPCRVTSPNITVTLKKFPLDTLIPDGKRIIWDSR |
| | VGFR- | KGFIISNATYKEIGLLTCEATVNGHLYKTNYLTHRQTNTIIDVVLSPSHGIELSVGEK |
| | CR1 | LVLNCTARTELNVGIDFNWEYPSSKHQHKKLVNRDLKTQSGSEMKKFLSTLTIDGV |
| | im- | TRSDQGLYTCAASSGLMTKKNSTFVRVHEKDKTHTCPPCPAPELLGGPSVFLFPPKP |
| | proved | KDTLMISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVD |
| | | KSRWQQGNVFSCSVMHEALHNHYTQKSLSLSPGKGGGGGGQCNAPEWLPFARPT |
| | | NLTDEFEFPIGTYLNYECRPGYSGRPFSIICLKNSVWTGAKDRCRRKSCRNPPDPVN |
| | | GMVHVIKGIQFGSQIKYSCTKGYRLIGSSSATCIISGDTVIWDNETPICDRIPCGLPPTI |
| | | TNGDFISTNRENFHYGSVVTYRCNPGSGGRKVFELVGEPSIYCTSNDDQVGIWSGP |
| | | APQCIIPNKCTPPNVENGILVSDNRSLFSLNEVVEFRCQPGFVMKGPRRVKCQALNK |
| | | WEPELPSCSRIFCPSPPVIPNGRHTGKPLEVFPFGKTVNYTCDPHPDRGTSFDLIGEST |
| | | IRCTSDPQGNGVWSSPAPRCGILGHCQAPDHFLFAKLKTQTNASDFPIGTSLKYECR |
| | | PEYYGRPFSITCLDNLVWSSPKDVCKRKSCKTPPDPVNGMVHVITDIQVGSRINYSC |
| | | TTGHRLIGHSSAECILSGNAAHWSTKPPICQRIPCGLPPTIANGDFISTNRENFHYGSV |
| | | VTYRCNPGSGGRKVFELVGEPSIYCTSNDDQVGIWSGPAPQCIIPNKCTPPNVENGIL |
| | | VSDNRSLFSLNEVVEFRCQPGFVMKGPRRVKCQALNKWEPELPSCSRIFCPSPPVIP |
| | | NGRHTGKPLEVFPFGKAVNYTCDPHPDRGTSFDLIGESTIRCTSDPQGNGVWSSPAP |
| | | RCGILGHCQAPDHFLFAKLKTQTNASDFPIGTSLKYECRPEYYGRPFSITCLDNLVW |
| | | SSPKDVCKRKSCKTPPDPVNGMVHVITDIQVGSRINYSCTTGHRLIGHSSAECILSGN |
| | | TAHWSTKPPICQRIPCGLPPTIANGDFISTNRENFHYGSVVTYRCNLGSRGRKVFEL |
| | | VGEPSIYCTSNDDQVGIWSGPAPQCII |
| 141 | CR2-CR1 | ctgcgcgctcgctcgctcactgaggccgcccgggcaaagcccgggcgtcgggcgacctttggtcgcccggcctcagtgagcga |
| | ITR to | gcgagcgcgcagagagggagtggccaactccatcactaggggttccttagtattaaacgcgtgcgacctagttattaatagtaatca |
| | ITR #1 | attacggggtcattagttcatagcccatatatggagttccgcgttacataacttacggtaaatggcccgcctggctgaccgcccaacg |
| | | acccccgcccattgacgtcaataatgacgtatgttcccatagtaacgccaatagggactttccattgacgtcaatgggtggactattta |
| | | cggtaaactgcccacttggcagtacatcaagtgtatcatatgccaagtacgccccctattgacgtcaatgacggtaaatggcccgcct |
| | | ggcattatgcccagtacatgaccttatgggactttcctacttggcagtacatctacgtattagtcatcgctattaccatggtcgaggtgag |
| | | ccccacgttctgcttcactctccccatctcccccccctccccacccccaattttgtatttatttattttttaattattttgtgcagcgatg |
| | | ggggcggggggggggggggggcgcgcgccaggcggggcggggcggggcgaggggcggggcggggcgaggcggagaggtg |
| | | cggcggcagccaatcagagcggcgcgctccgaaagtttccttttatggcgaggcggcggcggcggcggccctataaaaagcga |
| | | agcgcgcggggggggagtcgctgcgacgctgccttcgccccgtgccccgctccgccgccgcctcgcgccgcccgccccgg |
| | | ctctgactgaccgcgttactcccacaggtgagcggggggacggcccttctcctccgggctgtaattagcgcttggtttaatgacgg |
| | | cttgtttcttttctgtggctgcgtgaaagccttgaggggctccgggagctagagcctctgctaaccatgttcatgccttcttctttttcctac |
| | | agctcctgggcaacgtgctggttattgtgctgtctcatcattttggcaaagcggccgcaagcttgaattcatttaaatccagctagcgcc |
| | | accatgaggctcctggctttcctgagtctgctggccttggtgctgcaggagacagggacaatttcttgtggctctcctccgcctatcct |
| | | aaatggccggattagttattattctacccccattgctgttggtaccgtgataaggtacagttgttcaggtaccttccgcctcattggagaa |
| | | aaaagtctattatgcataactaaagacaaagtggatggaacctgggataaacctgctcctaaatgtgaatatttcaataaatattcttctt |
| | | gccctgagcccatagtaccaggaggatacaaaattagaggctctacaccctacagacatggtgattctgtgacatttgcctgtaaaac |
| | | caacttctccatgaacggaaacaagtctgtttggtgtcaagcaaataatatgtgggggccgacacgactaccaacctgtgtaagtgttt |
| | | tccctctcgagtgtccagcacttcctatgatccacaatggacatcacacaagtgagaatgttggctccattgctccaggattgtctgtga |
| | | cttacagctgtgaatctggttacttgcttgttggagaaaagatcattaactgtttgtcttcgggaaaatggagtgctgtcccccccacatg |
| | | tgaagaggcacgctgtaaatctctaggacgatttcccaatgggaaggtaaaggagcctccaattctccgggttggtgtaactgcaaa |
| | | ctttttctgtgatgaagggtatcgactgcaaggcccaccttctagtcggtgtgtaattgctggacagggagttgcttggaccaaaatgc |
| | | cagtatgtgaagaaatttttcaatgcaatgccccagaatggcttccatttgccaggcctaccaacctaactgatgaatttgagtttcccat |
| | | tgggacatatctgaactatgaatgccgccctggttattccggaagaccgttttctatcatctgcctaaaaaactcagtctggactggtgc |
| | | taaggacaggtgcagacgtaaatcatgtcgtaatcctccagatcctgtgaatggcatggtgcatgtgatcaaaggcatccagttcgga |
| | | tcccaaattaaatattcttgtactaaaggataccgactcattggttcctcgtctgccacatgcatcatctcaggtgatactgtcatttggga |
| | | taatgaaacacctatttgtgacagaattccttgtgggctaccccccaccatcaccaatggagatttcattagcaccaacagagagaatt |
| | | ttcactatggatcagtggtgacctaccgctgcaatcctggaagcggagggagaaaggtgtttgagcttgtgggtgagccctccatat |
| | | actgcaccagcaatgacgatcaagtgggcatctggagcggccccgcccctcagtgcattatacctaacaaatgcacgcctccaaat |
| | | gtggaaaatggaatattggtatctgacaacagaagcttattttccttaaatgaagttgtggagtttaggtgtcagcctggctttgtcatga |
| | | aaggaccccgccgtgtgaagtgccaggccctgaacaaatgggagccggagctaccaagctgctccagggtatgtcagccacctc |
| | | cagatgtcctgcatgctgagcgtacccaaagggacaaggacaacttttcacctgggcaggaagtgttctacagctgtgagcccggc |
| | | tacgacctcagaggggctgcgtctatgcgctgcacaccccagggagactggagccctgcagcccccacatgtgaagtgaaatcct |
| | | gtgatgacttcatgggccaacttcttaatggccgtgtgctatttccagtaaatctccagcttggagcaaaagtggattttgtttgtgatga |
| | | aggatttcaattaaaaggcagctctgctagttactgtgtcttggctggaatggaaagcctttggaatagcagtgttccagtgtgtgaaca |
| | | aatcttttgtccaagtcctccagttattcctaatgggagacacacaggaaaacctctggaagtctttccctttgggaaaacagtaaatta |
| | | cacatgcgacccccacccagacagagggacgagcttcgacctcattggagagagcaccatccgctgcacaagtgaccctcaagg |
| | | gaatggggtttggagcagccctgcccctcgctgtggaattctgggtcactgtcaagccccagatcattttctgtttgccaagttgaaaa |
| | | cccaaaccaatgcatctgactttcccattgggacatctttaaagtacgaatgccgtcctgagtactacgggaggccattctctatcacat |
| | | gtctagataacctggtctggtcaagtcccaaagatgtctgtaaacgtaaatcatgtaaaactcctccagatccagtgaatggcatggtg |
| | | catgtgatcacagacatccaggttggatccagaatcaactattcttgtactacagggcaccgactcattggtcactcatctgctgaatgt |
| | | atcctctcgggcaatgctgcccattggagcacgaagccgccaatttgtcaacgaattccttgtgggctaccccccaccatcgccaat |
| | | ggagatttcattagcaccaacagagagaattttcactatggatcagtggtgacctaccgctgcaatcctggaagcggagggagaaa |
| | | ggtgtttgagcttgtgggtgagccctccatatactgcaccagcaatgacgatcaagtgggcatctggagcggcccggcccctcagt |
| | | gcattatacctaacaaatagcaattgctgtgccttctagttgccagccatctgttgtttgcccctcccccgtgccttccttgaccctggaa |
| | | ggtgccactcccactgtcctttcctaataaaatgaggaaattgcatcgcattgtctgagtaggtgtcattctattctggggggtggggtg |
| | | gggcaggacagcaagggggaggattgggaagacaatagcaggcatgctggggatgcggtgggctctatggagctcgagagga |
| | | acccctagtgatggagttggccactccctctctgcgcgctcgctcgctcactgaggccgggcgaccaaaggtcgcccgacgcccg |
| | | ggctttgcccgggcggcctcagtgagcgagcgagcgcgcag |
| 142 | CR2-CR1 | ctgcgcgctcgctcgctcactgaggccgcccgggcaaagcccgggcgtcgggcgacctttggtcgcccggcctcagtgagcga |
| | ITR to | gcgagcgcgcagagagggagtggccaactccatcactaggggttccttagtattaaacgcgtgtcgacctagttattaatagtaatca |
| | ITR #2 | attacggggtcattagttcatagcccatatatggagttccgcgttacataacttacggtaaatggcccgcctggctgaccgcccaacg |
| | | acccccgcccattgacgtcaataatgacgtatgttcccatagtaacgccaatagggactttccattgacgtcaatgggtggactattta |
| | | cggtaaactgcccacttggcagtacatcaagtgtatcatatgccaagtacgccccctattgacgtcaatgacggtaaatggcccgcct |
| | | ggcattatgcccagtacatgaccttatgggactttcctacttggcagtacatctacgtattagtcatcgctattaccatggtcgaggtgag |
| | | ccccacgttctgcttcactctccccatctcccccccctccccacccccaattttgtatttatttattttttaattattttgtgcagcgatg |
| | | ggggcggggggggggggggggcgcgcgccaggcggggcggggcggggcgaggggcggggcggggcgaggcggagaggtg |
| | | cggcggcagccaatcagagcggcgcgctccgaaagtttccttttatggcgaggcggcggcggcggcggccctataaaaagcga |
| | | agcgcgcggggggggagtcgctgcgacgctgccttcgccccgtgccccgctccgccgccgcctcgcgccgcccgccccgg |
| | | ctctgactgaccgcgttactcccacaggtgagcggggggacggcccttctcctccgggctgtaattagcgcttggtttaatgacgg |
| | | cttgtttcttttctgtggctgcgtgaaagccttgaggggctccgggagctagagcctctgctaaccatgttcatgccttcttctttttcctac |
| | | agctcctgggcaacgtgctggttattgtgctgtctcatcattttggcaaagcggccgcaagcttgaattcatttaaatccagctagcgcc |
| | | accatgaggctcctggctttcctgagtctgctggccttggtgctgcaggagacagggacaatttcttgtggctctcctccgcctatcct |
| | | aaatggcaggattagttattattctacccccattgctgttggtacagtgataaggtacagttgttcaggtaccttccgcctcattggagaa |
| | | aaaagtctattatgcataactaaagacaaagtggatggaacctgggataaacctgctcctaaatgtgaatatttcaataaatattcttctt |
| | | gccctgagcccatagtaccaggaggatacaaaattagaggctctacaccctacagacatggtgattctgtgacatttgcctgtaaaac |
| | | caacttctccatgaatggaaacaagtctgtttggtgtcaagcaaataatatgtgggggccaacaaggctaccaacctgtgtaagtgttt |
| | | tccctcttgagtgtccagcacttcctatgatccacaatggacatcacacaagtgagaatgttggctccattgctccaggattgtctgtga |
| | | cttacagctgtgaatctggttacttgcttgttggagaaaagatcattaactgtttgtctagtggaaaatggagtgctgtcccccccacatg |
| | | tgaagaggcaaggtgtaaatctctaggaaggtttcccaatgggaaggtaaaggagcctccaattctcagggttggtgtaactgcaaa |
| | | ctttttctgtgatgaagggtataggctgcaaggcccaccttctagtaggtgtgtaattgctggacagggagttgcttggaccaaaatgc |
| | | cagtatgtgaagaaatttttcaatgcaatgccccagaatggcttccatttgccaggcctaccaacctaactgatgaatttgagtttcccat |
| | | tgggacatatctgaactatgaatgccgccctggttatagcggaagaccattttctatcatctgcctaaaaaactcagtctggactggtgc |
| | | taaggacaggtgcagaaggaaatcatgtaggaatcctccagatcctgtgaatggcatggtgcatgtgatcaaaggcatccagtttgg |
| | | atcccaaattaaatattcttgtactaaaggataccgcctcattggttcctcatctgccacatgcatcatctcaggtgatactgtcatttggg |
| | | ataatgaaacacctatttgtgacagaattccttgtgggctaccccccaccatcaccaatggagatttcattagcaccaacagagagaat |
| | | tttcactatggatcagtggtgacctaccgctgcaatcctggaagcggagggagaaaggtgtttgagcttgtgggtgagccctccatat |
| | | actgcaccagcaatgatgatcaagtgggcatctggagcggccccgcccctcagtgcattatacctaacaaatgcactcctccaaatg |
| | | tggaaaatggaatattggtatctgacaacagaagcttattttccttaaatgaagttgtggagtttaggtgtcagcctggctttgtcatgaa |
| | | aggaccccgcagggtgaagtgccaggccctgaacaaatgggagccagagctaccaagctgctccaggatcttttgtccaagtcct |
| | | ccagttattcctaatgggagacacacaggaaaacctctggaagtctttccctttgggaaaacagtaaattacacatgtgacccccacc |
| | | cagacagagggactagctttgacctcattggagagagcaccatccgctgcacaagtgaccctcaagggaatggggtttggagcag |
| | | ccctgcccctagatgtggaattctgggtcactgtcaagccccagatcattttctgtttgccaagttgaaaacccaaaccaatgcatctga |
| | | ctttcccattgggacatctttaaagtatgaatgccgccctgagtactatgggaggccattctctatcacatgtctagataacctggtctgg |
| | | tcaagtcccaaagatgtctgtaaaaggaaatcatgtaaaactcctccagatccagtgaatggcatggtgcatgtgatcacagacatcc |
| | | aggttggatccagaatcaactattcttgtactactgggcaccgcctcattggtcactcatctgctgaatgtatcctcagtggcaatgctg |
| | | cccattggagcactaagccgccaatttgtcaaagaattccttgtgggctaccccccaccattgccaatggagatttcattagcaccaac |
| | | agagagaattttcactatggatcagtggtgacctaccgctgcaatcctggaagcggagggagaaaggtgtttgagcttgtgggtgag |
| | | ccctccatatactgcaccagcaatgatgatcaagtgggcatctggagcggcccagcccctcagtgcattatacctaacaaatgcact |
| | | cctccaaatgtggaaaatggaatattggtatctgacaacagaagcttattttccttaaatgaagttgtggagtttaggtgtcagcctggct |
| | | ttgtcatgaaaggaccccgcagggtgaagtgccaggccctgaacaaatgggagccagagctaccaagctgctccaggatcttttgt |
| | | ccaagtcctccagttattcctaatgggagacacacaggaaaacctctggaagtctttccctttggaaaagcagtaaattacacatgtga |
| | | cccccacccagacagagggactagctttgacctcattggagagagcaccatccgctgcacaagtgaccctcaagggaatggggtt |
| | | tggagcagccctgcccctagatgtggaattctgggtcactgtcaagccccagatcattttctgtttgccaagttgaaaacccaaaccaa |
| | | tgcatctgactttcccattgggacatctttaaagtatgaatgccgccctgagtactatgggaggccattctctatcacatgtctagataac |
| | | ctggtctggtcaagtcccaaagatgtctgtaaaaggaaatcatgtaaaactcctccagatccagtgaatggcatggtgcatgtgatca |
| | | cagacatccaggttggatccagaatcaactattcttgtactactgggcaccgcctcattggtcactcatctgctgaatgtatcctctcag |
| | | gcaatactgcccattggagcactaagccgccaatttgtcaaagaattccttgtgggctacccccaaccattgccaatggagatttcatt |
| | | agcaccaacagagagaattttcactatggatcagtggtgacctaccgctgcaatcttggaagcagagggagaaaggtgtttgagctt |
| | | gtgggtgagccctccatatactgcaccagcaatgatgatcaagtgggcatctggagcggccccgcccctcagtgcattatatagcaa |
| | | ttgctgtgccttctagttgccagccatctgttgtttgcccctcccccgtgccttccttgaccctggaaggtgccactcccactgtcctttcc |
| | | taataaaatgaggaaattgcatcgcattgtctgagtaggtgtcattctattctggggggtggggggggcaggacagcaaggggga |
| | | ggattgggaagacaatagcaggcatgctggggatgcggtgggctctatggagctcgagaggaacccctagtgatggagttggcc |
| | | actccctctctgcgcgctcgctcgctcactgaggccgggcgaccaaaggtcgcccgacgcccgggctttgcccgggcggcctca |
| | | gtgagcgagcgagcgcgcag |
| 143 | IgG1 Fc | DKTHTCPPCPAPELLGGPSVFLFPPKPKDTLMISRTPEVTCVVVDVSHEDPEVKFNW |
| | portion | YVDGVEVHNAKTKPREEQYNSTYRVVSVLTVLHQDWLNGKEYKCKVSNKALPAP |
| | #2 | IEKTISKAKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYTQKSLSL |
| | | SPGK |
| 144 | IgG1 Fc | DKTHTCPPCPAPELLGGPSVFLFPPKPKDTLMISRTPEVTCVVVDVSHEDPEVQFNW |
| | portion | YVDGVEVHNAKTKPREEQFNSTFRVVSVLTVVHQDWLNGKEYKCKVSNKALPAPI |
| | #3 | EKTISKTKGQPREPQVYTLPPSREEMTKNQVSLTCLVKGFYPSDIAVEWESNGQPEN |
| | | NYKTTPPMLDSDGSFFLYSKLTVDKSRWQQGNVFSCSVMHEALHNHYTQKSLSLS |
| | | PGK |
| 145 | IgG1 Fc | DKTHTCPPCPAPELLGGPSVFLFPPKPKDTLMISRTPEVTCVVVDVSHEDPEVKFNW |
| | portion | YVDGVEVHNAKTKPREEQYNSTYRVVSVLTVLHQDWLNGKDYKCKVSNKALPAP |
| | #4 | MQKTISKAKGQPREPQVYTLPPSRDELTKNQVSLTCLVKGFYPRHIAVEWESNGQP |
| | | ENNYKTTPPVLDSDGSFFLYSKLTVDKSRWQQGNVFSCSVMHEALHNHYTQKSLS |
| | | LSPGK |
| 146 | VID #1 | GRPFVEMYSEIPEIIHMTEGRELVIPCRVTSPNITVTLKKFPLDTLIPDGKRIIWDSRK |
| | | GFIISNATYKEIGLLTCEATVNGHLYKTNYLTHRQTNTIIDVVLSPSHGIELSVGEKL |
| | | VLNCTARTELNVGIDFNWEYPSSKHQHKKLVNRDLKTQSGSEMKKFLSTLTIDGVT |
| | | RSDQGLYTCAASSGLMTKKNSTFVRVHEK |
| 147 | VID #2 | DTGRPFVEMYSEIPEIIHMTEGRELVIPCRVTSPNITVTLKKFPLDTLIPDGKRIIWDSR |
| | | KGFIISNATYKEIGLLTCEATVNGHLYKTNYLTHRQTNTIIDVVLSPSHGIELSVGEK |
| | | LVLNCTARTELNVGIDFNWEYPSSKHQHKKLVNRDLKTQSGSEMKKFLSTLTIDGV |
| | | TRSDQGLYTCAASSGLMTKKNSTFVRVHEK |
| 148 | CR1- | NKCTPPNVENGILVSDNRSLFSLNEVVEFRCQPGFVMKGPRRVKCQALNKWEPELP |
| | SCR11, | SCSRIFCPSPPVIPNGRHTGKPLEVFPFGKAVNYTCDPHPDRGTSFDLIGESTIRCTSD |
| | 14-17 | PQGNGVWSSPAPRCGILGHCQAPDHFLFAKLKTQTNASDFPIGTSLKYECRPEYYG |
| | | RPFSITCLDNLVWSSPKDVCKRKSCKTPPDPVNGMVHVITDIQVGSRINYSCTTGHR |
| | | LIGHSSAECILSGNTAHWSTKPPICQRIPCGLPPTIANGDFISTNRENFHYGSVVTYRC |
| | | NLGSRGRKVFELVGEPSIYCTSNDDQVGIWSGPAPQCII |
| 149 | CR1- | NKCTPPNVENGILVSDNRSLFSLNEVVEFRCQPGFVMKGPRRVKCQALNKWEPELP |
| | SCR11- | SCSRGGGGSGGGGSIFCPSPPVIPNGRHTGKPLEVFPFGKAVNYTCDPHPDRGTSFD |
| | linker- | LIGESTIRCTSDPQGNGVWSSPAPRCGILGHCQAPDHFLFAKLKTQTNASDFPIGTSL |
| | 14-17 | KYECRPEYYGRPFSITCLDNLVWSSPKDVCKRKSCKTPPDPVNGMVHVITDIQVGS |
| | | RINYSCTTGHRLIGHSSAECILSGNTAHWSTKPPICQRIPCGLPPTIANGDFISTNREN |
| | | FHYGSVVTYRCNLGSRGRKVFELVGEPSIYCTSNDDQVGIWSGPAPQCII |
| 150 | CR1- | GGGGSGGGGSGGGGSGGGGSGGGGSIFCPSPPVIPNGRHTGKPLEVFPFGKAVNYT |
| | Linker- | CDPHPDRGTSFDLIGESTIRCTSDPQGNGVWSSPAPRCGILGHCQAPDHFLFAKLKT |
| | SCR14- | QTNASDFPIGTSLKYECRPEYYGRPFSITCLDNLVWSSPKDVCKRKSCKTPPDPVNG |
| | 17 | MVHVITDIQVGSRINYSCTTGHRLIGHSSAECILSGNTAHWSTKPPICQRIPCGLPPTI |
| | (G4S)5 | ANGDFISTNRENFHYGSVVTYRCNLGSRGRKVFELVGEPSIYCTSNDDQVGIWSGP |
| | | APQCII |
| 151 | CR1- | GGGGSGGGGSGGGGSGGGGSGGGGSGGGGSGGGGSGGGGSGGGGSGGGGSGGG |
| | Linker- | GSGGGGSIFCPSPPVIPNGRHTGKPLEVFPFGKAVNYTCDPHPDRGTSFDLIGESTIR |
| | SCR14- | CTSDPQGNGVWSSPAPRCGILGHCQAPDHFLFAKLKTQTNASDFPIGTSLKYECRPE |
| | 17 | YYGRPFSITCLDNLVWSSPKDVCKRKSCKTPPDPVNGMVHVITDIQVGSRINYSCTT |
| | (G4S)12 | GHRLIGHSSAECILSGNTAHWSTKPPICQRIPCGLPPTIANGDFISTNRENFHYGSVVT |
| | | YRCNLGSRGRKVFELVGEPSIYCTSNDDQVGIWSGPAPQCII |
| 152 | CR1- | NKCTPPNVENGILVSDNRSLFSLNEVVEFRCQPGFVMKGPRRVKCQALNKWEPELP |
| | SCR11- | SCSRVCQPPPDVLHAERTQRDKDNFSPGQEVFYSCEPGYDLRGAASMRCTPQGDW |
| | 12, | SPAAPTCEVIFCPSPPVIPNGRHTGKPLEVFPFGKAVNYTCDPHPDRGTSFDLIGESTI |
| | 14-17 | RCTSDPQGNGVWSSPAPRCGILGHCQAPDHFLFAKLKTQTNASDFPIGTSLKYECRP |
| | | EYYGRPFSITCLDNLVWSSPKDVCKRKSCKTPPDPVNGMVHVITDIQVGSRINYSCT |
| | | TGHRLIGHSSAECILSGNTAHWSTKPPICQRIPCGLPPTIANGDFISTNRENFHYGSVV |
| | | TYRCNLGSRGRKVFELVGEPSIYCTSNDDQVGIWSGPAPQCII |
| 153 | CR1- | GHCQAPDHFLFAKLKTQTNASDFPIGTSLKYECRPEYYGRPFSITCLDNLVWSSPKD |
| | SCR15- | VCKRKSCKTPPDPVNGMVHVITDIQVGSRINYSCTTGHRLIGHSSAECILSGNTAHW |
| | 17 | STKPPICQRIPCGLPPTIANGDFISTNRENFHYGSVVTYRCNLGSRGRKVFELVGEPSI |
| | | YCTSNDDQVGIWSGPAPQCII |
| 154 | CR1- | NKCTPPNVENGILVSDNRSLFSLNEVVEFRCQPGFVMKGPRRVKCQALNKWEPELP |
| | SCR11- | SCSRVCQPPPDVLHAERTQRDKDNFSPGQEVFYSCEPGYDLRGAASMRCTPQGDW |
| | 14 | SPAAPTCEVKSCDDFMGQLLNGRVLFPVNLQLGAKVDFVCDEGFQLKGSSASYCV |
| | | LAGMESLWNSSVPVCEQIFCPSPPVIPNGRHTGKPLEVFPFGKAVNYTCDPHPDRGT |
| | | SFDLIGESTIRCTSDPQGNGVWSSPAPRCGI |
| 155 | CR1- | NKCTPPNVENGILVSDNRSLFSLNEVVEFRCQPGFVMKGPRRVKCQALNKWEPELP |
| | SCR11- | SCSRVCQPPPDVLHAERTQRDKDNFSPGQEVFYSCEPGYDLRGAASMRCTPQGDW |
| | 17 | SPAAPTCEVKSCDDFMGQLLNGRVLFPVNLQLGAKVDFVCDEGFQLKGSSASYCV |
| | | LAGMESLWNSSVPVCEQIFCPSPPVIPNGRHTGKPLEVFPFGKAVNYTCDPHPDRGT |
| | | SFDLIGESTIRCTSDPQGNGVWSSPAPRCGILGHCQAPDHFLFAKLKTQTNASDFPIG |
| | | TSLKYECRPEYYGRPFSITCLDNLVWSSPKDVCKRKSCKTPPDPVNGMVHVITDIQ |
| | | VGSRINYSCTTGHRLIGHSSAECILSGNTAHWSTKPPICQRIPCGLPPTIANGDFISTN |
| | | RENFHYGSVVTYRCNLGSRGRKVFELVGEPSIYCTSNDDQVGIWSGPAPQCII |
| 156 | G4S | GGGGS |
| | linker | |
| 157 | G4S | GGGGSGGGGS |
| | linker | |
| | #2 | |
| 158 | G4S | GGGGSGGGGSGGGGS |
| | linker | |
| | #3 | |
| 159 | G4S | GGGGSGGGGSGGGGSGGGGS |
| | linker | |
| | #4 | |
| 160 | G4S | GGGGSGGGGSGGGGSGGGGSGGGGS |
| | linker | |
| | #5 | |

* For Amino acids in Table 13:
Underlined = CR2 AA sequence;
Italics and Double Underlined = linker sequence; and
Bold = CR1 AA sequence.
** For Nucleotides in Table 13:
Bold and underlined = promoter sequence;
Italicized = human opticin signaling peptide sequence;
Underlined = CR2 sequence;
Underlined and italics = linker sequence;
Bold = CR1 protein; and
Bold and italicized = poly(A) sequence.

1. A fusion protein comprising
i) a complement receptor 2 (CR2) protein or a functional fragment thereof, and
ii) a complement receptor 1 (CR1) protein or a functional fragment thereof,
wherein the CR2 protein or functional fragment thereof has an amino acid sequence with at least 85%, at least 90%, at least 95%, at least 96%, at least 97%, at least 98%, at least 99%, or 100% identity to SEQ ID NO: 1 or SEQ ID NO: 2, and
wherein the CR1 protein or functional fragment thereof has an amino acid sequence with at least 95%, at least 96%, at least 97%, at least 98%, at least 99%, or 100% identity to SEQ ID NO: 3.

2. A polypeptide comprising a truncated complement receptor 1 (CR1) protein having an amino acid sequence with at least 85%, at least 90%, at least 95%, at least 96%, at least 97%, at least 98%, at least 99%, or 100% identity to any one of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

3. The polypeptide of claim 2 which is a fusion protein.

4. The polypeptide of claim 3, wherein the fusion protein comprises a complement receptor 2 (CR2) protein or a functional fragment thereof or a vascular endothelial growth factor (VEGF) binding domain or functional fragment thereof.

5-9. (canceled)

10. The fusion protein of claim 1, wherein the complement receptor 2 protein or functional fragment thereof has an amino acid sequence with at least 85%, at least 90%, at least 95%, at least 96%, at least 97%, at least 98%, at least 99%, or 100% identity to SEQ ID NO: 1.

11-14. (canceled)

15. A nucleic acid encoding the fusion protein of claim 1.

16-22. (canceled)

23. A vector comprising the nucleic acid of claim 15, wherein the vector is a non-viral vector or a viral vector.

24-25. (canceled)

26. An adeno-associated virus (AAV) vector comprising (i) a polynucleotide comprising two inverted terminal repeat (ITR) sequences and a promoter operably linked to a nucleic acid encoding a CR2-CR1 fusion protein, wherein the nucleic acid encoding the CR2-CR1 fusion comprises the nucleic acid of claim 15 and (ii) an AAV capsid.

27-33. (canceled)

34. The AAV vector of claim 26, wherein:
(a) the promoter comprises a CBA promoter, a CMV promoter, an EF-1a (Elongation Factor 1a) promoter, a RSV (Rous Sarcoma Virus) promoter, an Ubiquitin (UbC) promoter, a CAG promoter, a smCBA promoter or any combination thereof,
(b) the polynucleotide further comprises a kozak sequence:
(c) the polynucleotide further comprises a cytomegalovirus (CMV) immediate-early enhancer and/or promoter;
(d) the polynucleotide further comprises a poly(A) sequence;
(e) the polynucleotide further comprises an enhancer sequence; and/or
(f) the polynucleotide further comprises an intron sequence.

35. The AAV vector of claim 34, wherein the promoter is a smCBA promoter.

36-50. (canceled)

51. The AAV vector of claim 26, wherein the AAV capsid serotype is selected from AAV1, AAV2, AAV3, AAV4, AAV5, AAV6, AAV7, AAV8, AAVrh8, AAV9, AAV10, AAVrh10, AAV11, or AAV12.

52. The AAV vector of claim 26, wherein the AAV capsid serotype is selected from AAV2, AAV5, or AAV8.

53. The AAV vector of claim 51, wherein the AAV capsid is modified relative to the wild-type serotype.

54. A pharmaceutical composition comprising the AAV vector of claim 26 and a pharmaceutically acceptable excipient.

55. (canceled)

56. A method of treating a subject suffering from a complement-mediated disorder selected from:
(i) atypical hemolytic uremic syndrome, cold agglutinin disease, paroxysmal nocturnal hemoglobinuria, a transfusion reaction;
(ii) a transplant rejection, a transplant dysfunction;
(iii) a trauma, a surgery, myocardial infarction, ischemic stroke;
(iv) asthma, chronic obstructive pulmonary disease (COPD);
(v) arthritis; or
(vi) age-related macular degeneration (AIMD), diabetic retinopathy, glaucoma, or uveitis,
the method comprising administering the AAV vector of claim 26.

57. The method of claim 56, wherein the administration is to an eye of the subject.

58. The method of claim 56, wherein the administration is intravitreal, subretinal, or suprachoroidal injection.

59. (canceled)

60. The method of claim 56, wherein the complement-mediated disorder comprises age-related macular degeneration (AMD).

61. A method of treating a subject suffering from a complement-mediated disorder selected from:
(i) atypical hemolytic uremic syndrome, cold agglutinin disease, paroxysmal nocturnal hemoglobinuria, or a transfusion reaction;
(ii) a transplant rejection or a transplant dysfunction;
(iii) a trauma, a surgery, myocardial infarction, or ischemic stroke;
(iv) asthma, or chronic obstructive pulmonary disease (COPD);
(v) arthritis; or
(vi) age-related macular degeneration (AIMD), diabetic retinopathy, glaucoma, or uveitis,
the method comprising administering the fusion protein of claim 1.

62. A VID-CR1 fusion protein comprising the polypeptide of claim 4, wherein the fusion protein comprises i) a VEGF inhibiting domain (VID) and ii) a complement receptor 1 (CR1) protein or functional fragment thereof, wherein the CR1 protein or functional fragment thereof has an amino acid sequence with at least 95%, at least 96%, at least 97%, at least 98%, at least 99%, or 100% identity to any of SEQ ID NO: 3, and wherein the VID has an amino acid sequence with at least 85%, at least 90%, at least 95%, at least 96%, at least 97%, at least 98%, at least 99%, or 100% identity to any of SEQ ID NO: 136, 137, 146, or 147.

63-67. (canceled)

68. A nucleic acid encoding the VID-CR1 fusion protein of claim 62.

69-72. (canceled)

73. A method of treating a subject suffering from a) a solid tumor associated with angiogenesis or b) an ocular disease or condition associated with abnormal blood vessel in an eye, comprising administering to the subject an effective amount of the fusion protein of claim 62, a nucleic acid encoding the fusion protein, or an AAV vector comprising a nucleic acid encoding the fusion protein.

74-81. (canceled)

82. The method of claim 60, wherein the AMD comprises geographic atrophy (GA).

83. The fusion protein of claim 1 which comprises an amino acid sequence with at least 90%, at least 95%, at least 96%, at least 97%, at least 98%, at least 99%, or 100% identity to SEQ ID NO: 12.

84. A nucleic acid encoding a fusion protein comprising an amino acid sequence having at least 90%, at least 95%, at least 96%, at least 97%, at least 98%, at least 99%, or 100% identity to SEQ ID NO: 12.

85. An AAV vector comprising (i) the nucleic acid of claim 84 and (ii) an AAV capsid, wherein the AAV capsid serotype is selected from AAV2, AAV5, or AAV8.

86. A method of treating a subject suffering from age-related macular degeneration (AMD) comprising administering the AAV vector of claim 85 to the subject, wherein the administration is to an eye of the subject.

87. A fusion protein comprising
i) a complement receptor 2 (CR2) protein or a functional fragment thereof, and
ii) a complement receptor 1 (CR1) protein or a functional fragment thereof,
wherein the CR2 protein or functional fragment thereof has an amino acid sequence with at least 85%, at least 90%, at least 95%, at least 96%, at least 97%, at least 98%, at least 99%, or 100% identity to SEQ ID NO: 1 or SEQ ID NO: 2, and
wherein the CR1 protein or functional fragment thereof has an amino acid sequence with at least 85%, at least 90%, at least 95%, at least 96%, at least 97%, at least 98%, at least 99%, or 100% identity to any one of SEQ ID NO: 4, SEQ ID NO: 5, SEQ ID NO: 6, SEQ ID NO: 7, SEQ ID NO:
8, SEQ ID NO: 9, SEQ ID NO: 10, or SEQ ID NO: 11.

88. A nucleic acid encoding the fusion protein of claim 87.

89. A method of treating a subject suffering from a complement-mediated disorder selected from:
(i) atypical hemolytic uremic syndrome, cold agglutinin disease, paroxysmal nocturnal hemoglobinuria, a transfusion reaction;
(ii) a transplant rejection or a transplant dysfunction;
(iii) a trauma, a surgery, myocardial infarction, or ischemic stroke;
(iv) asthma or chronic obstructive pulmonary disease (COPD);
(v) arthritis; or
(vi) age-related macular degeneration (AMD), diabetic retinopathy, glaucoma, or uveitis,
the method comprising administering the fusion protein of claim 87, a nucleic acid encoding the fusion protein, or an AAV vector comprising a nucleic acid encoding the fusion protein.


SEQUENCE LISTING (160 sequences)

SEQ ID NO: 1 - protein, 255 aa, synthetic construct
  REGION 1..255: note = CR2 SCR1-4
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIF

SEQ ID NO: 2 - protein, 131 aa, synthetic construct
  REGION 1..131: note = CR2 SCR1-2
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFP

SEQ ID NO: 3 - protein, 847 aa, synthetic construct
  REGION 1..847: note = CR1 SCR1-4,7-11,14-17 (CR2CR1 #7)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 4 - protein, 867 aa, synthetic construct
  REGION 1..867: note = CR1 SCR1-4-linker-7-11-linker-14-17 (CR2CR1 #9)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRGGGGSGGGGSIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 5 - protein, 777 aa, synthetic construct
  REGION 1..777: note = CR1 SCR1-3-linker-7-10-linker-14-17 (CR2CR1 #10)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPGGGGSGGGGSGGGGSGGGGSGGGGSIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPGGGGSGGGGSGGGGSGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 6 - protein, 847 aa, synthetic construct
  REGION 1..847: note = CR1 SCR1-3-linker-7-10-linker-14-17 (CR2CR1 #10)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPGGGGSGGGGSGGGGSGGGGSGGGGSGGGGSGGGGSGGGGSGGGGSGGGGSGGGGSGGGGSIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPGGGGSGGGGSGGGGSGGGGSGGGGSGGGGSGGGGSGGGGSGGGGSGGGGSGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 7 - protein, 967 aa, synthetic construct
  REGION 1..967: note = CR1 SCR1-4,6-12,14-17 (CR2CR1 #12)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 8 - protein, 646 aa, synthetic construct
  REGION 1..646: note = CR1 SCR1-10 (CR2CR1 - #1)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNK

SEQ ID NO: 9 - protein, 584 aa, synthetic construct
  REGION 1..584: note = CR1 SCR1-3,8-10,15-17 (CR2CR1 - #3)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 10 - protein, 898 aa, synthetic construct
  REGION 1..898: note = CR1 SCR1-14 (CR2CR1 - #1)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGI

SEQ ID NO: 11 - protein, 1093 aa, synthetic construct
  REGION 1..1093: note = CR1 SCR1-17 (CR2CR1 #13)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 12 - protein, 1102 aa, synthetic construct
  REGION 1..1102: note = CR2CR1 #7
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 13 - protein, 1112 aa, synthetic construct
  REGION 1..1112: note = CR2CR1 #8
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 14 - protein, 1132 aa, synthetic construct
  REGION 1..1132: note = CR2CR1 #9
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRGGGGSGGGGSIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 15 - protein, 1102 aa, synthetic construct
  REGION 1..1102: note = CR2CR1 #10
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPGGGGSGGGGSGGGGSGGGGSGGGGSGGGGSGGGGSGGGGSGGGGSGGGGSGGGGSGGGGSIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPGGGGSGGGGSGGGGSGGGGSGGGGSGGGGSGGGGSGGGGSGGGGSGGGGSGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 16 - protein, 1032 aa, synthetic construct
  REGION 1..1032: note = CR2CR1 #10 - (G4S)5
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPGGGGSGGGGSGGGGSGGGGSGGGGSIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPGGGGSGGGGSGGGGSGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 17 - protein, 1112 aa, synthetic construct
  REGION 1..1112: note = CR2CR1 #11
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPGGGGSGGGGSGGGGSGGGGSGGGGSGGGGSGGGGSGGGGSGGGGSGGGGSGGGGSGGGGSIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPGGGGSGGGGSGGGGSGGGGSGGGGSGGGGSGGGGSGGGGSGGGGSGGGGSGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 18 - protein, 1042 aa, synthetic construct
  REGION 1..1042: note = CR2CR1 #11 - (G4S)5
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPGGGGSGGGGSGGGGSGGGGSGGGGSIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPGGGGSGGGGSGGGGSGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 19 - protein, 1128 aa, synthetic construct
  REGION 1..1128: note = CR2CR1 #12
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPGGGGSGGGGSGGGGSGGGGSGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 20 - protein, 1113 aa, synthetic construct
  REGION 1..1113: note = CR2CR1 #12 - (G4S)3
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPGGGGSGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 21 - protein, 898 aa, synthetic construct
  REGION 1..898: note = CR2CR1 #1
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCII

SEQ ID NO: 22 - protein, 901 aa, synthetic construct
  REGION 1..901: note = CR2CR1 #1-PNK
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNK

SEQ ID NO: 23 - protein, 911 aa, synthetic construct
  REGION 1..911: note = CR2CR1 #2
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNK

SEQ ID NO: 24 - protein, 839 aa, synthetic construct
  REGION 1..839: note = CR2CR1 #3
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 25 - protein, 849 aa, synthetic construct
  REGION 1..849: note = CR2CR1 #4
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 26 - protein, 1153 aa, synthetic construct
  REGION 1..1153: note = CR2CR1 #5
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGI

SEQ ID NO: 27 - protein, 1163 aa, synthetic construct
  REGION 1..1163: note = CR2CR1 #6
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGI

SEQ ID NO: 28 - protein, 1348 aa, synthetic construct
  REGION 1..1348: note = CR2CR1 #13
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 29 - DNA, 4405 bp, synthetic construct
  misc_feature 1..4405: note = CR2CR1 #7A
  source 1..4405: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 30 - DNA, 4405 bp, synthetic construct
  misc_feature 1..4405: note = CR2CR1 #7B
  source 1..4405: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctcttgagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgaataaaagatctttattttcattagatctgtgtgttggttttttgtgtg

SEQ ID NO: 31 - DNA, 4397 bp, synthetic construct
  misc_feature 1..4397: note = CR2CR1 #8A
  source 1..4397: mol_type = other DNA
catagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 32 - DNA, 4435 bp, synthetic construct
  misc_feature 1..4435: note = CR2CR1 #8B
  source 1..4435: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctcttgagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgaataaaagatctttattttcattagatctgtgtgttggttttttgtgtg

SEQ ID NO: 33 - DNA, 4610 bp, synthetic construct
  misc_feature 1..4610: note = CR2CR1 #8C
  source 1..4610: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctcttgagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 34 - DNA, 4495 bp, synthetic construct
  misc_feature 1..4495: note = CR2CR1 #9A
  source 1..4495: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggggtggaggtggatcgggaggtggtggatctatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggggtggaggtggatcgggaggtggtggatctatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 35 - DNA, 4495 bp, synthetic construct
  misc_feature 1..4495: note = CR2CR1 #9B
  source 1..4495: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctcttgagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggggtggaggtggatcaggaggtggtggatctatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggggtggaggtggatcaggaggtggtggatctatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgaataaaagatctttattttcattagatctgtgtgttggttttttgtgtg

SEQ ID NO: 36 - DNA, 4405 bp, synthetic construct
  misc_feature 1..4405: note = CR2CR1 #10A
  source 1..4405: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 37 - DNA, 4195 bp, synthetic construct
  misc_feature 1..4195: note = CR2CR1 #10B
  source 1..4195: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 38 - DNA, 4405 bp, synthetic construct
  misc_feature 1..4405: note = CR2CR1 #10C
  source 1..4405: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgaataaaagatctttattttcattagatctgtgtgttggttttttgtgtg

SEQ ID NO: 39 - DNA, 4195 bp, synthetic construct
  misc_feature 1..4195: note = CR2CR1 #10D
  source 1..4195: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggaagtggaggtggtggatctggtggaggtggatcaggaggtggtggatctggtggaggtggaagtatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggatcaggaggtggtggatctggtggaggtggaagtggaggtggtggatctggtggaggtggatcaatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgaataaaagatctttattttcattagatctgtgtgttggttttttgtgtg

SEQ ID NO: 40 - DNA, 4435 bp, synthetic construct
  misc_feature 1..4435: note = CR2CR1 #11A
  source 1..4435: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 41 - DNA, 4225 bp, synthetic construct
  misc_feature 1..4225: note = CR2CR1 #11B
  source 1..4225: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 42 - DNA, 4435 bp, synthetic construct
  misc_feature 1..4435: note = CR2CR1 #11C
  source 1..4435: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgaataaaagatctttattttcattagatctgtgtgttggttttttgtgtg

SEQ ID NO: 43 - DNA, 4225 bp, synthetic construct
  misc_feature 1..4225: note = CR2CR1 #11D
  source 1..4225: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggatcaggaggtggtggatctggtggaggtggaagtggaggtggtggatctggtggaggtggatcaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggaagtggaggtggtggatctggtggaggtggatcaggaggtggtggatctggtggaggtggaagtatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgaataaaagatctttattttcattagatctgtgtgttggttttttgtgtg

SEQ ID NO: 44 - DNA, 4483 bp, synthetic construct
  misc_feature 1..4483: note = CR2CR1 #12A
  source 1..4483: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctggtggaggtggatcgggaggtggtggatctggtggaggtggatcgggaggtggtggatctggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 45 - DNA, 4438 bp, synthetic construct
  misc_feature 1..4438: note = CR2CR1 #12B
  source 1..4438: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctggtggaggtggatcgggaggtggtggatctggtggaggtggatcgcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 46 - DNA, 4483 bp, synthetic construct
  misc_feature 1..4483: note = CR2CR1 #12C
  source 1..4483: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctggtggaggtggaagtggaggtggtggatctggtggaggtggaagtggaggtggtggatctggtggaggtggaagtggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaaggattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgaataaaagatctttattttcattagatctgtgtgttggttttttgtgtg

SEQ ID NO: 47 - DNA, 4438 bp, synthetic construct
  misc_feature 1..4438: note = CR2CR1 #12D
  source 1..4438: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctggtggaggtggaagtggaggtggtggatctggtggaggtggaagtcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaaggattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgaataaaagatctttattttcattagatctgtgtgttggttttttgtgtg

SEQ ID NO: 48 - DNA, 3977 bp, synthetic construct
  misc_feature 1..3977: note = CR2CR1 #1A
  source 1..3977: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 49 - DNA, 3977 bp, synthetic construct
  misc_feature 1..3977: note = CR2CR1 #1B
  source 1..3977: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtcatctggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtagatgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 50 - DNA, 3793 bp, synthetic construct
  misc_feature 1..3793: note = CR2CR1 #1C
  source 1..3793: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatatagcaattgaataaaagatctttattttcattagatctgtgtgttggttttttgtgtg

SEQ ID NO: 51 - DNA, 4007 bp, synthetic construct
  misc_feature 1..4007: note = CR2CR1 #2A
  source 1..4007: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 52 - DNA, 4007 bp, synthetic construct
  misc_feature 1..4007: note = CR2CR1 #2B
  source 1..4007: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtcatctggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtagatgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtacaactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 53 - DNA, 3791 bp, synthetic construct
  misc_feature 1..3791: note = CR2CR1 #3A
  source 1..3791: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcataattggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaaggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 54 - DNA, 3791 bp, synthetic construct
  misc_feature 1..3791: note = CR2CR1 #3B
  source 1..3791: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcataattggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaaggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 55 - DNA, 3821 bp, synthetic construct
  misc_feature 1..3821: note = CR2CR1 #4A
  source 1..3821: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcataattggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaaggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 56 - DNA, 3821 bp, synthetic construct
  misc_feature 1..3821: note = CR2CR1 #4B
  source 1..3821: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggaagtggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcataattggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaaggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 57 - DNA, 4733 bp, synthetic construct
  misc_feature 1..4733: note = CR2CR1 #5A
  source 1..4733: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaatttagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 58 - DNA, 4733 bp, synthetic construct
  misc_feature 1..4733: note = CR2CR1 #5B
  source 1..4733: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtcatctggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaagaaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagagtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagggtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaatttagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 59 - DNA, 4763 bp, synthetic construct
  misc_feature 1..4763: note = CR2CR1 #6A
  source 1..4763: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaatttagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 60 - DNA, 4763 bp, synthetic construct
  misc_feature 1..4763: note = CR2CR1 #6B
  source 1..4763: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtcatctggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaagaaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagagtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagggtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaatttagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 61 - DNA, 5143 bp, synthetic construct
  misc_feature 1..5143: note = CR2CR1 #13
  source 1..5143: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatagcaattgaataaaagatctttattttcattagatctgtgtgttggttttttgtgtg

SEQ ID NO: 62 - protein, 1092 aa, synthetic construct
  REGION 1..1092: note = human CR2 isoform 1
MGAAGLLGVFLALVAPGVLGISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFCPSPPPILNGRHIGNSLANVSYGSIVTYTCDPDPEEGVNFILIGESTLRCTVDSQKTGTWSGPAPRCELSTSAVQCPHPQILRGRMVSGQKDRYTYNDTVIFACMFGFTLKGSKQIRCNAQGTWEPSAPVCEKECQAPPNILNGQKEDRHMVRFDPGTSIKYSCNPGYVLVGEESIQCTSEGVWTPPVPQCKVAACEATGRQLLTKPQHQFVRPDVNSSCGEGYKLSGSVYQECQGTIPWFMEIRLCKEITCPPPPVIYNGAHTGSSLEDFPYGTTVTYTCNPGPERGVEFSLIGESTIRCTSNDQERGTWSGPAPLCKLSLLAVQCSHVHIANGYKISGKEAPYFYNDTVTFKCYSGFTLKGSSQIRCKADNTWDPEIPVCEKGCQSPPGLHHGRHTGGNTVFFVSGMTVDYTCDPGYLLVGNKSIHCMPSGNWSPSAPRCEETCQHVRQSLQELPAGSRVELVNTSCQDGYQLTGHAYQMCQDAENGIWFKKIPLCKVIHCHPPPVIVNGKHTGMMAENFLYGNEVSYECDQGFYLLGEKKLQCRSDSKGHGSWSGPSPQCLRSPPVTRCPNPEVKHGYKLNKTHSAYSHNDIVYVDCNPGFIMNGSRVIRCHTDNTWVPGVPTCIKKAFIGCPPPPKTPNGNHTGGNIARFSPGMSILYSCDQGYLLVGEALLLCTHEGTWSQPAPHCKEVNCSSPADMDGIQKGLEPRKMYQYGAVVTLECEDGYMLEGSPQSQCQSDHQWNPPLAVCRSRSLAPVLCGIAAGLILLTFLIVITLYVISKHRARNYYTDTSQKEAFHLEAREVYSVDPYNPAS

SEQ ID NO: 63 - protein, 1033 aa, synthetic construct
  REGION 1..1033: note = human CR2 isoform 2
MGAAGLLGVFLALVAPGVLGISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFCPSPPPILNGRHIGNSLANVSYGSIVTYTCDPDPEEGVNFILIGESTLRCTVDSQKTGTWSGPAPRCELSTSAVQCPHPQILRGRMVSGQKDRYTYNDTVIFACMFGFTLKGSKQIRCNAQGTWEPSAPVCEKECQAPPNILNGQKEDRHMVRFDPGTSIKYSCNPGYVLVGEESIQCTSEGVWTPPVPQCKVAACEATGRQLLTKPQHQFVRPDVNSSCGEGYKLSGSVYQECQGTIPWFMEIRLCKEITCPPPPVIYNGAHTGSSLEDFPYGTTVTYTCNPGPERGVEFSLIGESTIRCTSNDQERGTWSGPAPLCKLSLLAVQCSHVHIANGYKISGKEAPYFYNDTVTFKCYSGFTLKGSSQIRCKADNTWDPEIPVCEKETCQHVRQSLQELPAGSRVELVNTSCQDGYQLTGHAYQMCQDAENGIWFKKIPLCKVIHCHPPPVIVNGKHTGMMAENFLYGNEVSYECDQGFYLLGEKKLQCRSDSKGHGSWSGPSPQCLRSPPVTRCPNPEVKHGYKLNKTHSAYSHNDIVYVDCNPGFIMNGSRVIRCHTDNTWVPGVPTCIKKAFIGCPPPPKTPNGNHTGGNIARFSPGMSILYSCDQGYLLVGEALLLCTHEGTWSQPAPHCKEVNCSSPADMDGIQKGLEPRKMYQYGAVVTLECEDGYMLEGSPQSQCQSDHQWNPPLAVCRSRSLAPVLCGIAAGLILLTFLIVITLYVISKHRARNYYTDTSQKEAFHLEAREVYSVDPYNPAS

SEQ ID NO: 64 - protein, 2039 aa, synthetic construct
  REGION 1..2039: note = human CR1 isoform 1
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFTLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDALIVGTLSGTIFFILLIIFLSWIILKHRKGNNAHENPKEVAIHLHSQGGSSVHPRTLQTNEENSRVLP

SEQ ID NO: 65 - protein, 2489 aa, synthetic construct
  REGION 1..2489: note = human CR1 isoform 2
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFTLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDALIVGTLSGTIFFILLIIFLSWIILKHRKGNNAHENPKEVAIHLHSQGGSSVHPRTLQTNEENSRVLP

SEQ ID NO: 66 - protein, 2039 aa, synthetic construct
  REGION 1..2039: note = human CR1 isoform 3
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFTLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDALIVGTLSGTIFFILLIIFLSWIILKHRKGNNAHENPKEVAIHLHSQGGSSVHPRTLQTNEENSRVLP

SEQ ID NO: 67 - protein, 19 aa, synthetic construct
  REGION 1..19: note = human opticin signaling peptide
MRLLAFLSLLALVLQETGT

SEQ ID NO: 68 - DNA, 598 bp, synthetic construct
  misc_feature 1..598: note = cytomegalovirus (CMV) immediate-early enhancer and/or promoter
  source 1..598: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 69 - DNA, 380 bp, synthetic construct
  misc_feature 1..380: note = CMV enhancer
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 70 - DNA, 585 bp, synthetic construct
  misc_feature 1..585: note = Internal Ribosome Entry Site
  source 1..585: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccaca

SEQ ID NO: 71 - DNA, 49 bp, synthetic construct
  misc_feature 1..49: note = synthetic poly(A) sequence
  source 1..49: mol_type = other DNA
aataaaagatctttattttcattagatctgtgtgttggttttttgtgtg

SEQ ID NO: 72 - DNA, 224 bp, synthetic construct
  misc_feature 1..224: note = bovine growth hormone poly(A) sequence
  source 1..224: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 73 - DNA, 487 bp, synthetic construct
  misc_feature 1..487: note = human growth hormone poly(A) sequence
  source 1..487: mol_type = other DNA
gatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctg

SEQ ID NO: 74 - DNA, 19 bp, synthetic construct
  misc_feature 1..19: note = CR2-CR1 J Forward
  source 1..19: mol_type = other DNA
gacagggagttgcttggac

SEQ ID NO: 75 - DNA, 25 bp, synthetic construct
  misc_feature 1..25: note = CR2-CR1 J Reverse
  source 1..25: mol_type = other DNA
gggaaactcaaattcatcagttagg

SEQ ID NO: 76 - DNA, 24 bp, synthetic construct
  misc_feature 1..24: note = CR2-CR1 J Probe
  source 1..24: mol_type = other DNA
cagaatggcttccatttgccaggc

SEQ ID NO: 77 - DNA, 3309 bp, synthetic construct
  misc_feature 1..3309: note = CR2CR1 #7 (A) CR2-CR1 only
  source 1..3309: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 78 - DNA, 3309 bp, synthetic construct
  misc_feature 1..3309: note = CR2CR1 #7 (B) CR2-CR1 only
  source 1..3309: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctcttgagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 79 - DNA, 3339 bp, synthetic construct
  misc_feature 1..3339: note = CR2CR1 #8 (A) CR2-CR1 only
  source 1..3339: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 80 - DNA, 3339 bp, synthetic construct
  misc_feature 1..3339: note = CR2CR1 #8 (B) CR2-CR1 only
  source 1..3339: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctcttgagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 81 - DNA, 3339 bp, synthetic construct; identical to SEQ ID NO: 80
  misc_feature 1..3339: note = CR2CR1 #8 (C) CR2-CR1 only
  source 1..3339: mol_type = other DNA

SEQ ID NO: 82 - DNA, 3399 bp, synthetic construct
  misc_feature 1..3399: note = CR2CR1 #9 (A) CR2-CR1 only
  source 1..3399: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggggtggaggtggatcgggaggtggtggatctatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggggtggaggtggatcgggaggtggtggatctatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 83 - DNA, 3399 bp, synthetic construct
  misc_feature 1..3399: note = CR2CR1 #9 (B) CR2-CR1 only
  source 1..3399: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctcttgagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggggtggaggtggatcaggaggtggtggatctatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggggtggaggtggatcaggaggtggtggatctatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 84 - DNA, 3309 bp, synthetic construct
  misc_feature 1..3309: note = CR2CR1 #10 (A) CR2-CR1 only
  source 1..3309: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 85 - DNA, 3099 bp, synthetic construct
  misc_feature 1..3099: note = CR2CR1 #10 (B) CR2-CR1 only
  source 1..3099: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 86 - DNA, 3309 bp, synthetic construct
  misc_feature 1..3309: note = CR2CR1 #10 (C) CR2-CR1 only
  source 1..3309: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 87 - DNA, 3099 bp, synthetic construct
  misc_feature 1..3099: note = CR2CR1 #10 (D) CR2-CR1 only
  source 1..3099: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggaagtggaggtggtggatctggtggaggtggatcaggaggtggtggatctggtggaggtggaagtatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggatcaggaggtggtggatctggtggaggtggaagtggaggtggtggatctggtggaggtggatcaatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 88 - DNA, 3339 bp, synthetic construct
  misc_feature 1..3339: note = CR2CR1 #11 (A) CR2-CR1 only
  source 1..3339: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 89 - DNA, 3129 bp, synthetic construct
  misc_feature 1..3129: note = CR2CR1 #11 (B) CR2-CR1 only
  source 1..3129: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 90 - DNA, 3339 bp, synthetic construct
  misc_feature 1..3339: note = CR2CR1 #11 (C) CR2-CR1 only
  source 1..3339: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 91 - DNA, 3129 bp, synthetic construct
  misc_feature 1..3129: note = CR2CR1 #11 (D) CR2-CR1 only
  source 1..3129: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggatcaggaggtggtggatctggtggaggtggaagtggaggtggtggatctggtggaggtggatcaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggaagtggaggtggtggatctggtggaggtggatcaggaggtggtggatctggtggaggtggaagtatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 92 - DNA, 3387 bp, synthetic construct
  misc_feature 1..3387: note = CR2CR1 #12 (A) CR2-CR1 only
  source 1..3387: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctggtggaggtggatcgggaggtggtggatctggtggaggtggatcgggaggtggtggatctggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 93 - DNA, 3342 bp, synthetic construct
  misc_feature 1..3342: note = CR2CR1 #12 (B) CR2-CR1 only
  source 1..3342: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctggtggaggtggatcgggaggtggtggatctggtggaggtggatcgcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 94 - DNA, 3387 bp, synthetic construct
  misc_feature 1..3387: note = CR2CR1 #12 (C) CR2-CR1 only
  source 1..3387: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctggtggaggtggaagtggaggtggtggatctggtggaggtggaagtggaggtggtggatctggtggaggtggaagtggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaaggattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 95 - DNA, 3342 bp, synthetic construct
  misc_feature 1..3342: note = CR2CR1 #12 (D) CR2-CR1 only
  source 1..3342: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctggtggaggtggaagtggaggtggtggatctggtggaggtggaagtcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaaggattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 96 - DNA, 2706 bp, synthetic construct
  misc_feature 1..2706: note = CR2CR1 #1 (A) CR2-CR1 only
  source 1..2706: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatag

SEQ ID NO: 97 - DNA, 2706 bp, synthetic construct
  misc_feature 1..2706: note = CR2CR1 #1 (B) CR2-CR1 only
  source 1..2706: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtcatctggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtagatgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatag

SEQ ID NO: 98 - DNA, 2697 bp, synthetic construct
  misc_feature 1..2697: note = CR2CR1 #1 (C) CR2-CR1 only
  source 1..2697: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatatag

SEQ ID NO: 99 - DNA, 2736 bp, synthetic construct
  misc_feature 1..2736: note = CR2CR1 #2 (A) CR2-CR1 only
  source 1..2736: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatag

SEQ ID NO: 100 - DNA, 2736 bp, synthetic construct
  misc_feature 1..2736: note = CR2CR1 #2 (B) CR2-CR1 only
  source 1..2736: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtcatctggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtagatgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtacaactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatag

SEQ ID NO: 101 - DNA, 2520 bp, synthetic construct
  misc_feature 1..2520: note = CR2CR1 #3 (A) CR2-CR1 only
  source 1..2520: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcataattggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaaggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 102 - DNA, 2520 bp, synthetic construct
  misc_feature 1..2520: note = CR2CR1 #3 (B) 2 CR2-CR1 only
  source 1..2520: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcataattggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaaggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 103 - DNA, 2550 bp, synthetic construct
  misc_feature 1..2550: note = CR2CR1 #4 (A) CR2-CR1 only
  source 1..2550: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcataattggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaaggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 104 - DNA, 2550 bp, synthetic construct
  misc_feature 1..2550: note = CR2CR1 #4 (B) CR2-CR1 only
  source 1..2550: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggaagtggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcataattggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaaggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 105 - DNA, 3462 bp, synthetic construct
  misc_feature 1..3462: note = CR2CR1 #5 (A) CR2-CR1 only
  source 1..3462: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaatttag

SEQ ID NO: 106 - DNA, 3462 bp, synthetic construct
  misc_feature 1..3462: note = CR2CR1 #5 (B) CR2-CR1 only
  source 1..3462: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtcatctggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaagaaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagagtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagggtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaatttag

SEQ ID NO: 107 - DNA, 3492 bp, synthetic construct
  misc_feature 1..3492: note = CR2CR1 #6 (A) CR2-CR1 only
  source 1..3492: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttggtggaggtggatcgggaggtggtggatctcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaatttag

SEQ ID NO: 108 - DNA, 3492 bp, synthetic construct
  misc_feature 1..3492: note = CR2CR1 #6 (B) CR2-CR1 only
  source 1..3492: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctttggagtgtccagcacttcctatgatccacaatggacatcacacaagtgagaatgttggctccattgctccaggattgtctgtgacttacagctgtgaatctggttacttgcttgttggagaaaagatcattaactgtttgtcatctggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttggtggaggtggatcaggaggtggtggatctcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaagaaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagagtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagggtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaatttag

SEQ ID NO: 109 - DNA, 4047 bp, synthetic construct
  misc_feature 1..4047: note = CR2CR1 #13 CR2-CR1 only
  source 1..4047: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 110 - DNA, 765 bp, synthetic construct
  misc_feature 1..765: note = CR2 SCR1-4 #1
  source 1..765: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaattttt

SEQ ID NO: 111 - DNA, 765 bp, synthetic construct
  misc_feature 1..765: note = CR2 SCR1-4 #2
  source 1..765: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctcttgagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaattttt

SEQ ID NO: 112 - DNA, 393 bp, synthetic construct
  misc_feature 1..393: note = CR2 SCR1-2 #1
  source 1..393: mol_type = other DNA
atttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccct

SEQ ID NO: 113 - DNA, 393 bp, synthetic construct
  misc_feature 1..393: note = CR2 SCR1-2 #2
  source 1..393: mol_type = other DNA
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccct

SEQ ID NO: 114 - DNA, 2544 bp, synthetic construct
  misc_feature 1..2544: note = CR1 SCR1-4,7-11,14-17 (CR2CR1 #7) A
  source 1..2544: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 115 - DNA, 2544 bp, synthetic construct
  misc_feature 1..2544: note = CR1 SCR1-4,7-11,14-17 (CR2CR1 #7) B
  source 1..2544: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 116 - DNA, 2544 bp, synthetic construct
  misc_feature 1..2544: note = CR1 SCR1-4,7-11,14-17 (CR2CR1 #7) C
  source 1..2544: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 117 - DNA, 2604 bp, synthetic construct
  misc_feature 1..2604: note = CR1 SCR1-4-linker-7-11-linker-14-17 (CR2CR1 #9) A
  source 1..2604: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggggtggaggtggatcgggaggtggtggatctatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccaggggtggaggtggatcgggaggtggtggatctatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 118 - DNA, 2604 bp, synthetic construct
  misc_feature 1..2604: note = CR1 SCR1-4-linker-7-11-linker-14-17 (CR2CR1 #9) B
  source 1..2604: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggggtggaggtggatcaggaggtggtggatctatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggggtggaggtggatcaggaggtggtggatctatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 119 - DNA, 2544 bp, synthetic construct
  misc_feature 1..2544: note = CR1 SCR1-3-linker-7-10-linker14-17 (CR2CR1 #10) A
  source 1..2544: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctggtggaggtggatcgggaggtggtggatctatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 120 - DNA, 2334 bp, synthetic construct
  misc_feature 1..2334: note = CR1 SCR1-3-linker-7-10-linker14-17 (CR2CR1 #10) B
  source 1..2334: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctggtggaggtggatcgggaggtggtggatctggtggaggtggatcgggaggtggtggatctggtggaggtggatcgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 121 - DNA, 2544 bp, synthetic construct
  misc_feature 1..2544: note = CR1 SCR1-3-linker-7-10-linker14-17 (CR2CR1 #10) C
  source 1..2544: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 122 - DNA, 2334 bp, synthetic construct
  misc_feature 1..2334: note = CR1 SCR1-3-linker-7-10-linker14-17 (CR2CR1 #10) D
  source 1..2334: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggaagtggaggtggtggatctggtggaggtggatcaggaggtggtggatctggtggaggtggaagtatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggatcaggaggtggtggatctggtggaggtggaagtggaggtggtggatctggtggaggtggatcaatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 123 - DNA, 2544 bp, synthetic construct
  misc_feature 1..2544: note = CR1 SCR1-3-linker-7-10-linker14-17 (CR2CR1 #11) A
  source 1..2544: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggaagtggaggtggtggatctggtggaggtggatcaggaggtggtggatctggtggaggtggaagtggaggtggtggatctggtggaggtggatcaggaggtggtggatctggtggaggtggaagtggaggtggtggatctggtggaggtggatcaggaggtggtggatctatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 124 - DNA, 2334 bp, synthetic construct
  misc_feature 1..2334: note = CR1 SCR1-3-linker-7-10-linker14-17 (CR2CR1 #11) B
  source 1..2334: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctggtggaggtggatcaggaggtggtggatctggtggaggtggaagtggaggtggtggatctggtggaggtggatcaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctggtggaggtggaagtggaggtggtggatctggtggaggtggatcaggaggtggtggatctggtggaggtggaagtatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 125 - DNA, 2904 bp, synthetic construct
  misc_feature 1..2904: note = CR1 SCR1-4,6-12,14-17 (CR2CR1 #12) A
  source 1..2904: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 126 - DNA, 2904 bp, synthetic construct
  misc_feature 1..2904: note = CR1 SCR1-4,6-12,14-17 (CR2CR1 #12) B
  source 1..2904: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaaggattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 127 - DNA, 1941 bp, synthetic construct
  misc_feature 1..1941: note = CR1 SCR1-10 (CR2CR1 #1) A
  source 1..1941: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatag

SEQ ID NO: 128 - DNA, 1941 bp, synthetic construct
  misc_feature 1..1941: note = CR1 SCR1-10 (CR2CR1 #1) B
  source 1..1941: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatag

SEQ ID NO: 129 - DNA, 1932 bp, synthetic construct
  misc_feature 1..1932: note = CR1 SCR1-10 (CR2CR1 #1) C
  source 1..1932: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatatag

SEQ ID NO: 130 - DNA, 1941 bp, synthetic construct
  misc_feature 1..1941: note = CR1 SCR1-10 (CR2CR1 #2)
  source 1..1941: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctaggtgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaagaaaatcatgtaaaactcctccagatccagtgaatggcatggtgcatgtgatcacagacatccaggttggatccagaatcaactattcttgtacaactgggcaccgcctcattggtcactcatctgctgaatgtatcctcagtggcaatgctgcccattggagcactaagccgccaatttgtcaaaggattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatag

SEQ ID NO: 131 - DNA, 1755 bp, synthetic construct
  misc_feature 1..1755: note = CR1 SCR1-3,8-10,15-17 (CR2CR1 #3) A
  source 1..1755: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcataattggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaaggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 132 - DNA, 1755 bp, synthetic construct
  misc_feature 1..1755: note = CR1 SCR1-3,8-10,15-17 (CR2CR1 #3) B
  source 1..1755: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtagaaatcctccagatcctgtgaatggcatggtgcatgtgatcaaaggcatccagtttggatcccaaattaaatattcttgtactaaaggataccgcctcattggttccagttctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcataattggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaaggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatag

SEQ ID NO: 133 - DNA, 2697 bp, synthetic construct
  misc_feature 1..2697: note = CR1 SCR1-14 (CR2CR1 #5) A
  source 1..2697: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaatttag

SEQ ID NO: 134 - DNA, 2697 bp, synthetic construct
  misc_feature 1..2697: note = CR1 SCR1-14 (CR2CR1 #5) B
  source 1..2697: mol_type = other DNA
caatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaagaaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagagtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagggtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtacaactgggcaccgcctcattggtcactcatctgctgaatgtatcctctcaggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccagggtatgtcagccacctccagatgtcctgcatgctgagcgcacccaaagggacaaggacaacttttcacctgggcaggaagtgttctacagctgtgagcctggctatgacctcagaggggctgcatctatgcgctgcacaccccagggagactggagccctgcagcccccacatgtgaagtgaaatcctgtgatgacttcatgggccaacttcttaatggcagagtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctaggtgtggaatttag

SEQ ID NO: 135 - DNA, 3282 bp, synthetic construct
  misc_feature 1..3282: note = CR1 SCR1-17 (CR2CR1 #13)
  source 1..3282: mol_type = other DNA
caatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatatag

SEQ ID NO: 136 - protein, 103 aa, synthetic construct
  REGION 1..103: note = VGFR1 130-232
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDV

SEQ ID NO: 137 - protein, 101 aa, synthetic construct
  REGION 1..101: note = VGFR2 227-327 aa
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 138 - protein, 227 aa, synthetic construct
  REGION 1..227: note = IgG1 Fc portion #1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 6 aa, synthetic construct
  REGION 1..6: note = glycine linker
GGGGGG

SEQ ID NO: 140 - protein, 1284 aa, synthetic construct
  REGION 1..1284: note = Full VGFR-CR1 improved
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 141 - DNA, 4266 bp, synthetic construct
  misc_feature 1..4266: note = CR2-CR1 ITR to ITR #1
  source 1..4266: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgcgacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 142 - DNA, 4870 bp, synthetic construct
  misc_feature 1..4870: note = CR2-CR1 ITR to ITR #2
  source 1..4870: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagcggccgcaagcttgaattcatttaaatccagctagcgccaccatgaggctcctggctttcctgagtctgctggccttggtgctgcaggagacagggacaatttcttgtggctctcctccgcctatcctaaatggcaggattagttattattctacccccattgctgttggtacagtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaatggaaacaagtctgtttggtgtcaagcaaataatatgtgggggccaacaaggctaccaacctgtgtaagtgttttccctcttgagtgtccagcacttcctatgatccacaatggacatcacacaagtgagaatgttggctccattgctccaggattgtctgtgacttacagctgtgaatctggttacttgcttgttggagaaaagatcattaactgtttgtctagtggaaaatggagtgctgtcccccccacatgtgaagaggcaaggtgtaaatctctaggaaggtttcccaatgggaaggtaaaggagcctccaattctcagggttggtgtaactgcaaactttttctgtgatgaagggtataggctgcaaggcccaccttctagtaggtgtgtaattgctggacagggagttgcttggaccaaaatgccagtatgtgaagaaatttttcaatgcaatgccccagaatggcttccatttgccaggcctaccaacctaactgatgaatttgagtttcccattgggacatatctgaactatgaatgccgccctggttatagcggaagaccattttctatcatctgcctaaaaaactcagtctggactggtgctaaggacaggtgcagaaggaaatcatgtaggaatcctccagatcctgtgaatggcatggtgcatgtgatcaaaggcatccagtttggatcccaaattaaatattcttgtactaaaggataccgcctcattggttcctcatctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggccccgcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttgggaaaacagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctcagtggcaatgctgcccattggagcactaagccgccaatttgtcaaagaattccttgtgggctaccccccaccattgccaatggagatttcattagcaccaacagagagaattttcactatggatcagtggtgacctaccgctgcaatcctggaagcggagggagaaaggtgtttgagcttgtgggtgagccctccatatactgcaccagcaatgatgatcaagtgggcatctggagcggcccagcccctcagtgcattatacctaacaaatgcactcctccaaatgtggaaaatggaatattggtatctgacaacagaagcttattttccttaaatgaagttgtggagtttaggtgtcagcctggctttgtcatgaaaggaccccgcagggtgaagtgccaggccctgaacaaatgggagccagagctaccaagctgctccaggatcttttgtccaagtcctccagttattcctaatgggagacacacaggaaaacctctggaagtctttccctttggaaaagcagtaaattacacatgtgacccccacccagacagagggactagctttgacctcattggagagagcaccatccgctgcacaagtgaccctcaagggaatggggtttggagcagccctgcccctagatgtggaattctgggtcactgtcaagccccagatcattttctgtttgccaagttgaaaacccaaaccaatgcatctgactttcccattgggacatctttaaagtatgaatgccgccctgagtactatgggaggccattctctatcacatgtctagataacctggtctggtcaagtcccaaagatgtctgtaaaaggaaatcatgtaaaactcctccagatccagtgaatggcatggtgcatgtgatcacagacatccaggttggatccagaatcaactattcttgtactactgggcaccgcctcattggtcactcatctgctgaatgtatcctctcaggcaatactgcccattggagcactaagccgccaatttgtcaaagaattccttgtgggctacccccaaccattgccaatggagatttcattagcaccaacagagagaattttcactatggatcagtggtgacctaccgctgcaatcttggaagcagagggagaaaggtgtttgagcttgtgggtgagccctccatatactgcaccagcaatgatgatcaagtgggcatctggagcggccccgcccctcagtgcattatatagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 143 - protein, 227 aa, synthetic construct
  REGION 1..227: note = IgG1 Fc portion #2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 227 aa, synthetic construct
  REGION 1..227: note = IgG1 Fc portion #3
DKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKALPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 145 - protein, 227 aa, synthetic construct
  REGION 1..227: note = IgG1 Fc portion #4
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPMQKTISKAKGQPREPQVYTLPPSRDELTKNQVSLTCLVKGFYPRHIAVEWESNGQPENNYKTTPPVLDSDGSFFLYSKLTVDKSRWQQGNVFSCSVMHEALHNHYTQKSLSLSPGK

SEQ ID NO: 146 - protein, 202 aa, synthetic construct
  REGION 1..202: note = VID #1
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 147 - protein, 204 aa, synthetic construct
  REGION 1..204: note = VID #2
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 148 - protein, 326 aa, synthetic construct
  REGION 1..326: note = CR1 - SCR11,14-17
NKCTPPNVENGILVSDNRSLFSLNEVVEFRCQPGFVMKGPRRVKCQALNKWEPELPSCSRIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 149 - protein, 336 aa, synthetic construct
  REGION 1..336: note = CR1 - SCR11-linker-14-17
NKCTPPNVENGILVSDNRSLFSLNEVVEFRCQPGFVMKGPRRVKCQALNKWEPELPSCSRGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 150 - protein, 291 aa, synthetic construct
  REGION 1..291: note = CR1 - Linker-SCR14-17 (G4S)5
GGGGSGGGGSGGGGSGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 151 - protein, 326 aa, synthetic construct
  REGION 1..326: note = CR1 - Linker-SCR14-17 (G4S)12
GGGGSGGGGSGGGGSGGGGSGGGGSGGGGSGGGGSGGGGSGGGGSGGGGSGGGGSGGGGSIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 152 - protein, 386 aa, synthetic construct
  REGION 1..386: note = CR1 - SCR11-12,14-17
NKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 153 - protein, 194 aa, synthetic construct
  REGION 1..194: note = CR1 - SCR15-17
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 154 - protein, 254 aa, synthetic construct
  REGION 1..254: note = CR1 - SCR11-14
NKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGI

SEQ ID NO: 155 - protein, 449 aa, synthetic construct
  REGION 1..449: note = CR1 - SCR11-17
NKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 156 - protein, 5 aa, Synthetic construct
  REGION 1..5: note = G4S Linker
GGGGS

SEQ ID NO: 157 - protein, 10 aa, Synthetic construct
  REGION 1..10: note = G4S linker #2
GGGGSGGGGS

SEQ ID NO: 158 - protein, 15 aa, Synthetic construct
  REGION 1..15: note = G4S linker #3
GGGGSGGGGSGGGGS

SEQ ID NO: 159 - protein, 20 aa, Synthetic construct
  REGION 1..20: note = G4S linker #4
GGGGSGGGGSGGGGSGGGGS

SEQ ID NO: 160 - protein, 25 aa, Synthetic construct
  REGION 1..25: note = G4S linker #5
GGGGSGGGGSGGGGSGGGGSGGGGS